SEQUENCE LISTING (1050 sequences)

SEQ ID NO: 1 - protein, 1244 aa, synthetic construct
  source 1..1244: note = ART1
METFSGFTNLYPLSKTLRFRLIPVGETLKHFIDSGILEEDQHRAESYVKVKAIIDDYHRAYIENSLSGFELPLESTKFNSLEEYYLYHNIRNKTEEIQNLSSKVRTNLRKQVVAQLTKNEIFKRIDKKELIQSDLIDFVKNEPDANEKIALISEFRNFTVYFKGFHENRRNMYSDEEKSTSIAFRLIHENLPKFIDNMEVFAKIQNTSISENFDAIQKELCPELVTLCEMFKLGYFNKTLSQKQIDAYNTVIGGKTTSEGKKIKGLNEYINLYNQQHKQEKLPKMKLLFKQILSDRESASWLPEKFENDSQVVGAIVNFWNTIHDTVLAEGGLKTIIASLGSYGLEGIFLKNDLQLTDISQKATGSWGKISSEIKQKIEVMNPQKKKESYETYQERIDKIFKSYKSFSLAFINECLRGEYKIEDYFLKLGAVNSSSLQKENHFSHILNTYTDVKEVIGLYSESTDTKLIQDNDSIQKIKQFLDAVKDLQAYVKPLLGNGDETGKDERFYGDLIEYWSLLDLITPLYNMVRNYVTQKPYSVDKIKINFQNPTLLNGWDLNKETDNTSVILRRDGKYYLAIMNNKSRKVFLKYPSGTDRNCYEKMEYKLLPGANKMLPKVFFSKSRINEFMPNERLLSNYEKGTHKKSGTCFSLDDCHTLIDFFKKSLDKHEDWKNFGFKFSDTSTYEDMSGFYKEVENQGYKLSFKPIDATYVDQLVDEGKIFLFQIYNKDFSEHSKGTPNMHTLYWKMLFDETNLGDVVYKLNGEAEVFFRKASINVSHPTHPANIPIKKKNLKHKDEERILKYDLIKDKRYTVDQFQFHVPITMNFKADGNGNINQKAIDYLRSASDTHIIGIDRGERNLLYLVVIDGNGKICEQFSLNEIEVEYNGEKYSTNYHDLLNVKENERKQARQSWQSIANIKDLKEGYLSQVIHKISELMVKYNAIVVLEDLNAGFMRGRQKVEKQVYQKFEKKLIEKLNYLVFKKQSSDLPGGLMHAYQLANKFESFNTLGKQSGFLFYIPAWNTSKMDPVTGFVNLFDVKYESVDKAKSFFSKFDSIRYNVERDMFEWKFNYGEFTKKAEGTKTDWTVCSYGNRIITFRNPDKNSQWDNKEINLTENIKLLFERFGIDLSSNLKDEIMQRTEKEFFIELISLFKLVLQMRNSWTGTDIDYLVSPVCNENGEFFDSRNVDETLPQNADANGAYNIARKGMILLDKIKKSNGEKKLALSITNREWLSFAQGCCKNG

SEQ ID NO: 2 - protein, 1197 aa, synthetic construct
  source 1..1197: note = ART2
MLSNFTNQYQLSKTLRFELKPVGDTLKHIEKSGLIAQDEIRSQEYQEVKTIIDKYHKAFIDEALQNVVLSNLEEYEALFFERNRDEKAFEKLQAVLRKEIVAHFKQHPQYKTLFKKELIKADLKNWQELSDAEKELVSHFDNFTTYFTGFHENRANMYTDEAKHSSIAYRIIHENLPIFLTNKKLFETIKQKAPHLAQETQDALLEYLSGAIVEDMFELSYFNHLLSQTHIDLYNQMIGGVKQDSLKIQGLNEKINLYRQANGLSKRELPNLKPLHKQILSDRETLSWLPESFESDEELMQGVQAYFESEVLAFECCDGKVNLLEKLPELLHQTQDYDFSKVYFKNDLALTAASQAIFKDYRIIKEALWEVNKPKKSKDLVADEEKFFNKKNSYFSIEQIDGALNSAQLSANMMHYFQSESTKVIEQIQLTYNDWKRNSSNKELLKAFLDALLSYQRLLKPLNAPNDLEKDVAFYAYFDAYFTSLCGVVKLYDKVRNFMTKKPYSLEKFKLNFENSTLLDGWDVNKESDNTAILFRKEGLYYLGIMNKKYNKVFRNISSSQDEGYQKIDYKLLPGANKMLPKVFFSDKNKEYFKPNAKLLERYKAGEHKKGDNFDLDFCHELIDFFKTSIEKHQDWKHFAYQFSPTESYEDLSGFYREVEQQGYKISYKNIAASFIDTLVAEGKLYFFQIYNKDFSPYSKGTPNMHTLYWRALFDEKNLADVIYKLNGQAEIFFRKKSIEYSQEKLQKGHHHEMLKDKFAYPIIKDRRFAFDKFQFHVPITLNFKAEGNENITPKTFEYIRSNPDNIKVIGIDRGERHLLYLSLIDAEGKIVEQFTLNQIINSYNGKDHVIDYHAKLDAKEKDRDKARKEWGTVENIKELKEGYLSHVIHKIATLIIEHGAVVAMEDLNFGFKRGRFKVEKQVYQKFEKALIDKLNYLVDKKKEPHKLGGLLNALQLTSKFQSFEKMGKQNGFLFYVPAWNTSKIDPVTGFVNLFDTRYASVEKSKAFFTKFQSICYNEAKDYFELVFDYNDFTEKAKETRSEWTLCTYGERIVSFRNAEKNHQWDSKTIHLTTEFKNLFGELHGNDVKEYILEQNSVEFFKSLIYLLKITLQMRNSITGTDIDYLVSPVADEAGNFYDSRKADTSLPKDADANGAYNIARKGLMLMHRIQNAEDLKKVNLAISNRDWLRNAQGLDK

SEQ ID NO: 3 - protein, 1252 aa, synthetic construct
  source 1..1252: note = ART3
MIDLKQFIGIYPVSKTLRFELRPVGKTQEWIEKNRVLEGDEQKAADYPVVKKLIDDYHKVCIHDSLNHVHFDWEPLKDAIEIFQKTKSDEAKKRLEAEQAMMRKKIAAAIKDFKHFKELTAATPSDLITSVLPEFSDDGSLKSFRGFATYFSGFQENRNNIYSQEAISTGVPYRLVHDNFPKFLSDLEVFERIKSTCPEVINQASAELQPFLEGVMIDDIFSLDFYNSLLTQNGIDFFNQVIGGVSEKDKQKYRGINEFSNLYRQQHKEIAASKKAMTMIPLFKQILSDRDTLSYIPAQIRTEDELVSSITQFYDHITHFEHDGKTINVLSEIVALLGKLDTYDPNGICITARKLTDISQKVYGKWSVIEEKMKEKAIQQYGDISVAKNKKKVDAFLSRKAYSLSDLCFDEEISFSRYYSELPQTLNAISGYWLQFNEWCKSDEKQKFLNNQTGTEVVKSLLDAMMELFHKCSVLVMPEEYEVDKSFYNEFLPLYEELDTLFLLYNKVRNYLTQKPSDVKKFKLNFESPSLASGWDQNKEMKNNAILLFKDGKSYLGVLNAKNKAKIKDAKGDVSSSSYKKMIYKLLSDPSKDLPHKIFAKGNLDFYKPSEYILEGRELGKYKKGPNFDKKFLHDFIDFYKAAISIDPDWSKFNFQYSPTESYDDIGMFFSEIKKQAYKIRFTDISEAQVNEWVDNGQLYLFQLYNKDYAEGAHGRKNLHTLYWENLFTDENLSNLVLKLNGQAELFCRPQSIKKPVSHKIGSKMLNRRDKSGMPIPESIYRSLYQYYNGKKKESELTVAEKQYIDQVIVKDVTHEIIKDRRYTRQEYFFHVPLTFNANADGNEYINEHVLNYLKDNPDVNIIGIDRGERHLIYLTLINQRGEILKQKTFNVVNSYNYQAKLEQREKERDEARKSWDSVGKIKDLKEGFLSAVIHEITNMMIENNAIVVLEDLNFGFKRGRFKVERQVYQKFEKMLIDKLNYLSFKDREAGEEGGILRGYQMAQKFISFQRLGKQSGFLFYIPAAYTSKIDPVSGFVNHFNFSDITNAEKRKDFLMKMDRIEMKNGNIEFTFDYRKFKTFQTDYQNVWTVSTFGKRIVMRIDEKGYKKMVDYEPTNDIIKAFKNKGILLSEGSDLKALIAEIEANATNAGFYSTLLYAFQKTLQMRNSNAVTEEDYILSPVAKDGHQFCSTDEANKGKDAQGNWVSKLPVDADANGAYHIALKGLYLLRNPETKKIENEKWLQFMVEKPYLE

SEQ ID NO: 4 - protein, 1287 aa, synthetic construct
  source 1..1287: note = ART4
MSYNREKMEEKELGKNQNFQEFIGVSPLQKTLRNELIPTETTKKNIAQLDLLTEDEVRAQNREKLKEMMDDYYRDVIDSTLRGELLIDWSYLFSCMRNHLSENSKESKRELERTQDSVRSQIHDKFAERADFKDMFGASIITKLLPTYIKQNSKYSERYDESVKIMKLYGKFTTSLTDYFETRKNIFSKEKISSAVGYRIVEENAEIFLQNQNAYDRICKIAGLDLHGLDNEITAYVDGKTLKEVCSDEGFAKVITQGGIDRYNEAIGAVNQYMNLLCQKNKALKPGQFKMKRLHKQILCKGTTSFDIPKKFENDKQVYDAVNSFTEIVTKNNDLKRLLNITQNANDYDMNKIYVVADAYSMISQFISKKWNLIEECLLDYYSDNLPGKGNAKENKVKKAVKEETYRSVSQLNEVIEKYYVEKTGQSVWKVESYISSLAEMIKLELCHEIDNDEKHNLIEDDEKISEIKELLDMYMDVFHIIKVFRVNEVLNFDETFYSEMDEIYQDMQEIVPLYNHVRNYVTQKPYKQEKYRLYFHTPTLANGWSKSKEYDNNAIILVREDKYYLGILNAKKKPSKEIMAGKEDCSEHAYAKMNYYLLPGANKMLPKVFLSKKGIQDYHPSSYIVEGYNEKKHIKGSKNFDIRFCRDLIDYFKECIKKHPDWNKFNFEFSATETYEDISVFYREVEKQGYRVEWTYINSEDIQKLEEDGQLFLFQIYNKDFAVGSTGKPNLHTLYLKNLFSEENLRDIVLKLNGEAEIFFRKSSVQKPVIHKCGSILVNRTYEITESGTTRVQSIPESEYMELYRYFNSEKQIELSDEAKKYLDKVQCNKAKTDIVKDYRYTMDKFFIHLPITINFKVDKGNNVNAIAQQYIAEQEDLHVIGIDRGERNLIYVSVIDMYGRILEQKSFNLVEQVSSQGTKRYYDYKEKLQNREEERDKARKSWKTIGKIKELKEGYLSSVIHEIAQMVVKYNAIIAMEDLNYGFKRGRFKVERQVYQKFETMLISKLNYLADKSQAVDEPGGILRGYQMTYVPDNIKNVGRQCGIIFYVPAAYTSKIDPTTGFINAFKRDVVSTNDAKENFLMKFDSIQYDIEKGLFKFSFDYKNFATHKLTLAKTKWDVYTNGTRIQNMKVEGHWLSMEVELTTKMKELLDDSHIPYEEGQNILDDLREMKDITTIVNGILEIFWLTVQLRNSRIDNPDYDRIISPVLNNDGEFFDSDEYNSYIDAQKAPLPIDADANGAFCIALKGMYTANQIKENWVEGEKLPADCLKIEHASWLAFMQGERG

SEQ ID NO: 5 - protein, 1295 aa, synthetic construct
  source 1..1295: note = ART5
MSAVFKIKESTMKDFTHQYSLSKTLRFELKPVGETAERIEDFKNQGLKSIVEEDRQRAEDYKKMKRILDDYHKEFIEEVLNDDIFTANEMESAFEVYRKYMASKNDDKLKKEITEIFTDLRKKIAKAFENKSKEYCLYKGDFSKLINEKKTGKDKGPGKLWYWLKAKADAGVNEFGDGQTFEQAEEALAKFNNFSTYFTGFNQNRDNIYTDAEQQTAISYRVINENMTRYFDNCIRYSSIENKYPELVKQLEPLSGKFAPGNYKDYLSQTAIDIYNEAVGHKSDDINAKGINQFINEYRQRNSIKGRELPIMSVLYKQILSDINKDLIIDKFENAGELLDAVKTLHRELTDKKILLKIKQTLNEFLTEDNSEDIYIKSGTDLTAVSNAIWGEWSVIPKALEMYAENITDMNAKAREKWLKREAYHLKTVQEAIEAYLKDNEEFETRNISEYFTNFKSGENDLIQVVQSAYAKMESIFGIEDFHKDRRPVTESGEPGEGFRQVELVREYLDSLINVEHFIKPLHMFRSGKPIELEDCNSNFYDPLNEAYKELDVVFGIYNKVRNYVTQKPYSKDKFKINFQNSTLLDGWDVNKESANSSVLLLKNGKYYLGVMKQGASNILNYRPEPSDSKNKINAKKQLSEIALAGATDDYYEKMIYKLLPDPAKMLPKVFFSAKNIEFYNPSQEIIYIRENGLFKKDAGDKESLKKWIGFMKTSLLKHPEWGSYFNFEFEPAEDYQDISIFYKQVAEQGYSVTFDKIKTSYIEEKVASGELYLFEIYNKDFSPHSKGRPNLHTMYWKSLFEKENLQNLVTKLNGEAEVFFRQHSIKRNEKVVHRANRPIQNKNPLTEKKQSIFEYDLVKDRRFTKDKFFLHCPITLNFKEAGPGRFNDKVNKYIAGNPDIRIIGIDRGERHLLYYSLIDQSGRIVEQGTLNQITSTLNSGGREIPKTTDYRGLLDTKEKERDKARKSWSMIENIKELKSGYLSHIVHKLAKLMVKNNAVVVLEDLNFGFKRGRFKVEKQVYQKFEKALIEKLNYLVFKDARPAEPGHYLNAYQLTAPLESFKKLGKQSGFIYYVPAWNTSKIDPVTGFVNQFYIEKNSMQYLKNFFGKFDSIRFNPDKNYFEFGFDYKNFHNKAAKSKWTICTHGDKRSWYNRKQRKLEIHNVTENLASLLSGKGINFADGGSIKDKILSVDDASFFKSLAFNFKLTAQLRHTFEDNGEEIDCIISPVAAADGTFFCSETAKKLNMELPHDADANGAYNIARKGLMVLRQIRESGKPKPISNADWLDFAQQNED

SEQ ID NO: 6 - protein, 1245 aa, synthetic construct
  source 1..1245: note = ART6
MQERKKISHLTHRNSVQKTIRMQLNPVGKTMDYFQAKQILENDEKLKENYQKIKEIADRFYRNLNEDVLSKTGLDKLKDYAEIYYHCNTDAERKRLDECASELRKEIVKNFKNRDEYNKLFNKKMIEIVLPQHLKNEDEKEVVASFKNFTTYFTGFFTNRKNMYSDGEESTAIAYRCINENLPKHLDNVKAFEKAISKLSKNAIDDLDATYSGLCGTNLYDVFTVDYFNFLLPQSGITEYNKIIGGYTTSDGTKVKGINEYINLYNQQVSKRDKIPNLKILYKQILSESEKVSFIPPKFEDDNELLSAVSEFYANDETFDGMPLKKAIDETKLLFGNLDNSSLNGIYIQNDRSVTNLSNSMFGSWSVIEDLWNKNYDSVNSNSRIKDIQKREDKRKKAYKAEKKLSLSFLQVLISNSENDEIREKSIVNYYKTSLMQLTDNLSDKYNEAAPLLNKSYANEKGLKNDDKSISLIKNFLDAIKEIEKFIKPLSETNITGEKNDLFYSQFTPLLDNISRIDILYDKVRNYVTQKPFSTDKIKLNFGNSQLLNGWDRNKEKDCGAVWLCRDEKYYLAIIDKSNNSILENIDFQDCDENDCYEKIIYKLLPGPNKMLPKVFFSEKCKKLLSPSDEILKIRKNGTFKKGDKFSLDDCHKLIDFYKESFKKYPNWLIYNFKFKNTNEYNDIREFYNDVASQGYNISKMKIPTSFIDKLVDEGKIYLFQLYNKDFSPHSKGTPNLHTLYFKMLFDERNLEDVVYKLNGEAEMFYRPASIKYDKPTHPKNTPIKNKNTLNDKKTSTFPYDLIKDKRYTKWQFSLHFPITMNFKAPDRAMINDDVRNLLKSCNNNFIIGIDRGERNLLYVSVIDSNGAIIYQHSLNIIGNKFKGKTYETNYREKLATREKERTEQRRNWKAIESIKELKEGYISQAVHVICQLVVKYDAIIVMEKLTDGFKRGRTKFEKQVYQKFEKMLIDKLNYYVDKKLDPDEGGGLLHAYQLTNKLESFDKLGMQSGFIFYVRPDFTSKIDPVTGFVNLLYPRYENIDKAKDMISRFDDIGYNAGEDFFEFDIDYDKFPKTASDYRKRWTICTNGERIEAFRNPAKNNEWSYRTIILAEKFKELFDNNSINYRDSDDLKAEILSQTKGKFFEDFFKLLRLTLQMRNSNPETGEDRILSPVKDKNGNFYDSSKYDEKSKLPCDADANGAYNIARKGLWIVEQFKKSDNVSTVGPVIHNDKWLKFVQENDMANN

SEQ ID NO: 7 - protein, 1281 aa, synthetic construct
  source 1..1281: note = ART7
MNILKENYMKEIKELTGLYSLTKTIGVELKPVGKTQELIEAKKLIEQDDQRAEDYKIVKDIIDRYHKDFIDKCLNCVKIKKDDLEKYVSLAENSNRDAEDFDKIKTKMRNQITEAFRKNSLFTNLFKKNLIKEYLPAFVSEEEKSVVNKFSKFTTYFDAFNDNRKNLYSGDAKSGTIAYRLIHENLPMFLDNIASFNAISGIGVNEYFSSIETEFTDTLEGKRLTEFFQIDFFNNTLTQKKIGNYNYIVGAVNKAVNLYKQQHKTVRVPLLKPLYKMILSDRVTPSWLPERFESDEEMLTAIKAAYESLREVLVGDNDESLRNLLLNIEHYDLEHIYIANDSGLTSISQKIFGCYDTYTLAIKDQLQRDYPATKKQREAPDLYDERIDKLYKKVGSFSIAYLNRLVDAKGHFTINEYYKQLGAYCREEGKEKDDFFKRIDGAYCAISHLFFGEHGEIAQSDSDVELIQKLLEAYKGLQRFIKPLLGHGDEADKDNEFDAKLRKVWDELDIITPLYDKVRNWLSRKIYNPEKIKLCFENNGKLLSGWVDSRTKSDNGTQYGGYIFRKKNEIGEYDFYLGISADTKLFRRDAAISYDDGMYERLDYYQLKSKTLLGNSYVGDYGLDSMNLLSAFKNAAVKFQFEKEVVPKDKENVPKYLKRLKLDYAGFYQILMNDDKVVDAYKIMKQHILATLTSSIRVPAAIELATQKELGIDELIDEIMNLPSKSFGYFPIVTAAIEEANKRENKPLFLFKMSNKDLSYAATASKGLRKGRGTENLHSMYLKALLGMTQSVFDIGSGMVFFRHQTKGLAETTARHKANEFVANKNKLNDKKKSIFGYEIVKNKRFTVDKYLFKLSMNLNYSQPNNNKIDVNSKVREIISNGGIKNIIGIDRGERNLLYLSLIDLKGNIVMQKSLNILKDDHNAKETDYKGLLTEREGENKEARRNWKKIANIKDLKRGYLSQVVHIISKMMVEYNAIVVLEDLNPGFIRGRQKIERNVYEQFERMLIDKLNFYVDKHKGANETGGLLHALQLTSEFKNFKKSEHQNGCLFYIPAWNTSKIDPATGFVNLFNTKYTNAVEAQEFFSKFDEIRYNEEKDWFEFEFDYDKFTQKAHGTRTKWTLCTYGMRLRSFKNSAKQYNWDSEVVALTEEFKRILGEAGIDIHENLKDAICNLEGKSQKYLEPLMQFMKLLLQLRNSKAGTDEDYILSPVADENGIFYDSRSCGDQLPENADANGAYNIARKGLMLIEQIKNAEDLNNVKFDISNKAWLNFAQQKPYKNG

SEQ ID NO: 8 - protein, 1293 aa, synthetic construct
  source 1..1293: note = ART8
MAKENIFNELTGKYQLSKTLRLELKPVGNTQQMLKDEDVFEKDRIIREKYRETRPHFDRLHREFIEQALKNQKLSDLGKYFQCLAKLQNNKKDKEAQEEFKRISQNLRKEVNDLFKIDPLFGEGVFALLKEKYGEKDDAFLREQDGQYVLDENKKKISIFDSWKGFTGYFTKFQETRKNFYKDDGTATAVATRIIDQNLKRFCENIQIFKSIQKKVDFKEVEDNFSVDLEDIFSLGFYSSCFLQEGIDVYNKILGGEPKTTGEKLRGLNELINRYRQDHKGEKLPFFKMLDKQILSEKEKFIESIEDDEELLKTLKEFYSSAEEKTTVLKELFNDFIKNNENYDLSEIYISREALNTISHRWVSAATLPEFEKSVYEVMKKDKPSGLSFDKDDNSYKFPDFIALSYIKGSFEKLSGEKLWKDGYFRDETRNGDKGFLIGNESLWTQFIKIFEFEFNSLFEAKNTERSVGYYHFKKDFEKIITNDFSVNPEDKVIIREFADNVLAIYQMAKYFAIEKKRKWMDQYDTGDFYNHPDFGYKTKFYDNAYEKIVKARMLLQSYLTKKPFSTDKWKLNFECGYLLNGWSSSFNTYGSLLFRTGNEYYLGVVNGSALRTEKIKRLTGNITEANSCHKMVYDFQKPDNKNVPRIFIRSKGDKFAPAVSELNLPVDSILEIYDKGLFKTENKNSPFFKPSLKKLIDYFKLGFSRHASYKHYQFKWKDSSEYKNISEFYNDTIRSCYQIKWEELNFEEVKKLTNSKDLFLFQIYNKDFSEKSTGNKNLHSIYFDGLFLDNNINAQDGVILKLSGGGEIFFRPKTDVKKLGSRTDTKGKLVIKNKRYSQDKIFLHFPIELNYSNTQESNFNKLVRNFLADNPDINIIGVDRGEKHLIYYAGIDQKGNTLKDKDDKDVLGSLNEINGVNYYKLLEERAKAREKARQDWQNIQGIKDLKMGYISLVVRKLADLIIEYNAILVLEDLNMRFKQIHGGIEKSVYQQLEKALIEKLNFLVNKGEKDPERAGHLLRAYQLTAPFSTFKDMGKQTGVLFYTQASYTSKTCPQCGFRPNIKLHFDNLENAKKMLEKINIVYKDNHFEIGYKVSDFTKTEKTSRGNILYGDRQGKDTFVISSKAAIRYKWFARNIKNNELNRGESLKEHTEKGVTIQYDITECLKILYEKNGIDHSGDITKQSIRSELPAKFYKDLLFYLYLLTNTRSSISGTEIDYINCPDCGFHSEKGFNGCIFNGDANGAYNIARKGMLILKKINQYKDQHHTMDKMGWGDLFIGIEEWDKYTQVVSRS

SEQ ID NO: 9 - protein, 1273 aa, synthetic construct
  source 1..1273: note = ART9
MKEIKELTGLYSLTKTIGVELKPVGKTQELIEAKKLIEQDDQRAEDYKIVKDIIDRYHKDFIDKCLNCVKIKKDDLEKYVSLAENSNRDAEDFDKIKTKMRNQITEAFRKNSLFTNLFKKNLIKEYLPAFVSEEEKSVVNKFSKFTTYFDAFNDNRKNLYSGDAKSGTIAYRLIHENLPMFLDNIASFNAISGIGVNEYFSSIETEFTDTLEGKRLTEFFQIDFFNNTLTQKKIGNYNYIVGAVNKAVNLYKQQHKTVRVPLLKPLYKMILSDRVTPSWLPERFESDEEMLTAIKAAYESLREVLVGDNDESLRNLLLNIEHYDLEHIYIANDSGLTSISQKIFGCYDTYTLAIKDQLQRDYPATKKQREAPDLYDERIDKLYKKVGSFSIAYLNRLVDAKGHFTINEYYKQLGAYCREEGKEKDDFFKRIDGAYCAISHLFFGEHGEIAQSDSDVELIQKLLEAYKGLQRFIKPLLGHGDEADKDNEFDAKLRKVWDELDIITPLYDKVRNWLSRKIYNPEKIKLCFENNGKLLSGWVDSRTKSDNGTQYGGYIFRKKNEIGEYDFYLGISADTKLFRRDAAISYDDGMYERLDYYQLKSKTLLGNSYVGDYGLDSMNLLSAFKNAAVKFQFEKEVVPKDKENVPKYLKRLKLDYAGFYQILMNDDKVVDAYKIMKQHILATLTSSIRVPAAIELATQKELGIDELIDEIMNLPSKSFGYFPIVTAAIEEANKRENKPLFLFKMSNKDLSYAATASKGLRKGRGTENLHSMYLKALLGMTQSVFDIGSGMVFFRHQTKGLAETTARHKANEFVANKNKLNDKKKSIFGYEIVKNKRFTVDKYLFKLSMNLNYSQPNNNKIDVNSKVREIISNGGIKNIIGIDRGERNLLYLSLIDLKGNIVMQKSLNILKDDHNAKETDYKGLLTEREGENKEARRNWKKIANIKDLKRGYLSQVVHIISKMMVEYNAIVVLEDLNPGFIRGRQKIERNVYEQFERMLIDKLNFYVDKHKGANETGGLLHALQLTSEFKNFKKSEHQNGCLFYIPAWNTSKIDPATGFVNLFNTKYTNAVEAQEFFSKFDEIRYNEEKDWFEFEFDYDKFTQKAHGTRTKWTLCTYGMRLRSFKNSAKQYNWDSEVVALTEEFKRILGEAGIDIHENLKDAICNLEGKSQKYLEPLMQFMKLLLQLRNSKAGTDEDYILSPVADENGIFYDSRSCGDQLPENADANGAYNIARKGLMLIEQIKNAEDLNNVKFDISNKAWLNFAQQKPYKNG

SEQ ID NO: 10 - protein, 1300 aa, synthetic construct
  source 1..1300: note = ART10
MNFQPFFQKFVHLYPISKTLRFELIPQGATQKFISEKQVLLQDEIRARKYPEMKQAIDGYHKDFIQRALSNIDSQVFEQALNTFEDLFLRSQAERATDAYKKDFETAQTKLRELIVHSFEKGEFKQEYKSLFDKNLITNLLKPWVEQQNQIGDSNYTYHEDFNKFTTYFLGFHENRKNIYSKDPHKTALAYRLIHENLPKFLENNKILLKIQNDHPSLWEQLQTLNQTMPQLFDGWDFSQLMQVSFFSNTLTQTGIDQYNTIIGGISEGENRQKIQGINELINLYNQKQDKKNRVAKLKQLYKQILSDRSTLSFLPEKFVDDTELYHAINMFYLEHLHHQSMINGHSYTLLERVQLLINELANYDLSKVYLAPNQLSTVSHQMFGDFGYIGRALNYYYMQVIQPDYEQLLASAKTTKKIEATEKLKTIFLDTPQSLVVIQAAIDEYIQLQPSTKPHTQLTDFIISLLKQYETVADDQSIKVINVFSDIEGKYSCIKGLVNTKSESKREVLQDEKLATDIKAFMDAVNNVIKLLKPFSLNEKLVASVEKDARFYSDFEEIYQSLLIFVPLYNKVRNYITQKPYSTEKFKLNFNKPTLLSGWDANKEADNLSILLRKNGNYYLAIMDTAKGANKAFEPKTLNQLKVDDTTDCYEKMVYKLLSGPSKMFPKAFKAKNNEGNYYPTPELLTSYNNNEHLKNDKNFTLASLHAYIDWCKEYINRNPSWHQFNFKFSPTQSFQDISQFYSEVSSQSYKVHFQTIPSDYIDQLVAEGKLYLFQIYNKDFSPNAKGKENLHTLYFKALFSDENLKQPVFKLSGEAEMFYRPASLQLANTTIHKAGEPMAAKNPLTPNATRTLAYDIIKDRRFTTDKYLLHVPISLNFHAQESMSIKKHNDLVRQMIKHNHQDLHVIGIDRGEKHLLYVSVIDLKGNIVYQESLNSIKSEAQNFETPYHQLLQHREEGRAQARTAWGKIENIKELKDGYLSQVVHRIQQLILKYNAIVMLEDLNFGFKRGRFKIEKQIYQKFEKALIHKLNYVVDKSTQADELGGVRKAYQLTAPFESFEKLGKQSGVLFYVPAWNTSKIDPVTGFVDLLKPKYENLDKAQAFFNAFDSIHYNAQKNYFEFKVNLKQFAGLKAQAAQAEWTICSYGDERHVYQKKNAQQGETVIVNVTEELKVLFAKNNIEVAQSVELKETICTQTQVDFFKRLMWLLQVLLALRYSSSKDKLDYILSPVANAQGEFFDSRHASVQLPQDSDANGAYHIALKGLWVIEQLKAADNLDKVKLAISNDDWLHFAQQKPYLA

SEQ ID NO: 11 - protein, 1205 aa, synthetic construct
  source 1..1205: note = ART11
MYYQGLTKLYPISKTIRNELIPVGKTLEHIRMNNILEADIQRKSDYERVKKLMDDYHKQLINESLQDVHLSYVEEAADLYLNASKDKDIVDKFSKCQDKLRKEIVNLLKSHENFPKIGNKEIIKLLQSLSDTEKDYNALDSFSKFYTYFTSYNEVRKNLYSDEEKSSTAAYRLINENLPKFLDNIKAYSIAKSAGVRAKELTEEEQDCLFMTETFERTLTQDGIDNYNELIGKLNFAINLYNQQNNKLKGFRKVPKMKELYKQILSEREASFVDEFVDDEALLTNVESFSAHIKEFLESDSLSRFAEVLEESGGEMVYIKNDTSKTTFSNIVFGSWNVIDERLAEEYDSANSKKKKDEKYYDKRHKELKKNKSYSVEKIVSLSTETEDVIGKYIEKLQADIIAIKETREVFEKVVLKEHDKNKSLRKNTKAIEAIKSFLDTIKDFERDIKLISGSEHEMEKNLAVYAEQENILSSIRNVDSLYNMSRNYLTQKPFSTEKFKLNFNRATLLNGWDKNKETDNLGILLVKEGKYYLGIMNTKANKSFVNPPKPKTDNVYHKVNYKLLPGPNKMLPKVFFAKSNLEYYKPSEDLLAKYQAGTHKKGENFSLEDCHSLISFFKDSLEKHPDWSEFGFKFSDTKKYDDLSGFYREVEKQGYKITYTDIDVEYIDSLVEKDELYLFQIYNKDFSPYSKGNYNLHTLYLTMLFDERNLRNVVYKLNGEAEVFYRPASIGKDELIIHKSGEEIKNKNPKRAIDKPTSTFEYDIVKDRRYTKDKFMLHIPVTMNFGVDETRRFNEVVNDAIRGDDKVRVIGIDRGERNLLYVVVVDSDGTILEQISLNSIINNEYSIETDYHKLLDEKEGDRDRARKNWTTIENIKELKEGYLSQVVNVIAKLVLKYDAIICLEDLNFGFKRGRQKVEKQVYQKFEKMLIDKLNYLVIDKSRSQENPEEVGHVLNALQLTSKFTSFKELGKQTGIIYYVPAYLTSKIDPTTGFANLFYVKYESVEKSKDFFNRFDSICFNKVAGYFEFSFDYKNFTDRACGMRSKWKVCTNGERIIKYRNEEKNSSFDDKVIVLTEEFKKLFNEYGIAFNDCMDLTDAINAIDDASFFRKLTKLFQQTLQMRNSSADGSRDYIISPVENDNGEFFNSEKCDKSKPKDADANGAFNIARKGLWVLEQLYNSSSGEKLNLAMTNAEWLEYAQQHTI

SEQ ID NO: 12 - protein, 1306 aa, synthetic construct
  source 1..1306: note = ART12
MAKNFEDFKRLYPLSKTLRFEAKPIGATLDNIVKSGLLEEDEHRAASYVKVKKLIDEYHKVFIDRVLDNGCLPLDDKGDNNSLAEYYESYVSKAQDEDAIKKFKEIQQNLLSIIAKKLTDDKAYANLFGNKLIESYKDKADKTKLIDSDLIQFINTAESTQLVSMSQDEAKELVKEFWGFTTYFEGFFKNRKNMYTPEEKSTGIAYRLINENLPKFIDNMEAFKKAIARPEIQANMEELYSNFSEYLNVESIQEMFLLDYYNMLLTQKQIDVYNAIIGGKTDDEHDVKIKGINEYINLYNQQHKDDKLPKLKALFKQILSDRNAISWLPEEFNSDQEVLNAIKDCYERLAENVLGDKVLKSLLGSLADYSLDGIFIRNDLQLTDISQKMFGNWGVIQNAIMQNIKHVAPARKHKESEEDYEKRIAGIFKKADSFSISYINDCLNEADPNNAYFVENYFATFGAVNTPTMQRENLFALVQNAYTEVAALLHSDYPTVKHLAQDKANVSKIKALLDAIKSLQHFVKPLLGKGDESDKDERFYGELASLWAELDTVTPLYNMIRNYMTRKPYSQKKIKLNFENPQLLGGWDANKEKDYATIILRRNGLYYLAIMDKDSRKLLGKAMPSDGECYEKMVYKFFKDVTTMIPKCSTQLKDVQAYFKVNTDDYVLNSKAFNRPLTITKEVFDLNNVLYGKYKKFQKGYLTATGDNVGYTHAVNVWIKFCMDFLDSYDSTCIYDFSSLKPESYLSLDSFYQDVNLLLYKLSFTDVSASFIDQLVEEGKMYLFQIYNKDFSEYSKGTPNMHTLYWKALFDERNLADVVYKLNGQAEMFYRKKSIENTHPTHPANHPILNKNKDNKKKESLFEYDLIKDRRYTVDKFMFHVPITMNFKSSGSENINQDVKAYLRHADDMHIIGIDRGERHLLYLVVIDLQGNIKEQFSLNEIVNDYNGNTYHTNYHDLLDVREDERLKARQSWQTIENIKELKEGYLSQVIHKITQLMVRYHAIVVLEDLSKGFMRSRQKVEKQVYQKFEKMLIDKLNYLVDKKTDVSTPGGLLNAYQLTCKSDSSQKLGKQSGFLFYIPAWNTSKIDPVTGFVNLLDTHSLNSKEKIKAFFSKFDAIRYNKDKKWFEFNLDYDKFGKKAEDTRTKWTLCTRGMRIDTFRNKEKNSQWDNQEVDLTTEMKSLLEHYYIDIHGNLKDAISTQTDKAFFTGLLHILKLTLQMRNSITGTETDYLVSPVADENGIFYDSRSCGDQLPENADANGAYNIARKGLMLVEQIKDAEDLDNVKFDISNKAWLNFAQQKPYKNG

SEQ ID NO: 13 - protein, 1306 aa, synthetic construct
  source 1..1306: note = ART13
MAKNFEDFKRLYSLSKTLRFEAKPIGATLDNIVKSGLLDEDEHRAASYVKVKKLIDEYHKVFIDRVLDDGCLPLENKGNNNSLAEYYESYVSRAQDEDAKKKFKEIQQNLRSVIAKKLTEDKAYANLFGNKLIESYKDKEDKKKIIDSDLIQFINTAESTQLDSMSQDEAKELVKEFWGFVTYFYGFFDNRKNMYTAEEKSTGIAYRLVNENLPKFIDNIEAFNRAITRPEIQENMGVLYSDFSEYLNVESIQEMFQLDYYNMLLTQKQIDVYNAIIGGKTDDEHDVKIKGINEYINLYNQQHKDDKLPKLKALFKQILSDRNAISWLPEEFNSDQEVLNAIKDCYERLAENVLGDKVLKSLLGSLADYSLDGIFIRNDLQLTDISQKMFGNWGVIQNAIMQNIKRVAPARKHKESEEDYEKRIAGIFKKADSFSISYINDCLNEADPNNAYFVENYFATFGAVNTPTMQRENLFALVQNAYTEVAALLHSDYPTVKHLAQDKANVSKIKALLDAIKSLQHFVKPLLGKGDESDKDERFYGELASLWAELDTVTPLYNMIRNYMTRKPYSQKKIKLNFENPQLLGGWDANKEKDYATIILRRNGLYYLAIMDKDSRKLLGKAMPSDGECYEKMVYKFFKDVTTMIPKCSTQLKDVQAYFKVNTDDYVLNSKAFNKPLTITKEVFDLNNVLYGKYKKFQKGYLTATGDNVGYTHAVNVWIKFCMDFLNSYDSTCIYDFSSLKPESYLSLDAFYQDANLLLYKLSFARASVSYINQLVEEGKMYLFQIYNKDFSEYSKGTPNMHTLYWKALFDERNLADVVYKLNGQAEMFYRKKSIENTHPTHPANHPILNKNKDNKKKESLFDYDLIKDRRYTVDKFMFHVPITMNFKSVGSENINQDVKAYLRHADDMHIIGIDRGERHLLYLVVIDLQGNIKEQYSLNEIVNEYNGNTYHTNYHDLLDVREEERLKARQSWQTIENIKELKEGYLSQVIHKITQLMVRYHAIVVLEDLSKGFMRSRQKVEKQVYQKFEKMLIDKLNYLVDKKTDVSTPGGLLNAYQLTCKSDSSQKLGKQSGFLFYIPAWNTSKIDPVTGFVNLLDTHSLNSKEKIKAFFSKFDAIRYNKDKKWFEFNLDYDKFGKKAEDTRTKWTLCTRGMRIDTFRNKEKNSQWDNQEVDLTTEMKSLLEHYYIDIHGNLKDAISAQTDKAFFTGLLHILKLTLQMRNSITGTETDYLVSPVADENGIFYDSRSCGNQLPENADANGAYNIARKGLMLIEQIKNAEDLNNVKFDISNKAWLNFAQQKPYKNG

SEQ ID NO: 14 - protein, 1274 aa, synthetic construct
  source 1..1274: note = ART14
MAKNFEDFKRLYSLSKTLRFEAKPIGATLDNIVKSDLLDEDEHRAASYVKVKKLIDEYHKVFIDRVLDDGCLPLENKGNNNSLAEYYESYVSRAQDEDAKKKFKEIQQNLRSVIAKKLTEDKAYANLFGNKLIESYKDKEDKKKIIDSDLIQFINTAESTQLDSMSQDEAKELVKEFWGFVTYFYGFFDNRKNMYTAEEKSTGIAYRLVNENLPKFIDNIEAFNRAITRPEIQENMGVLYSDFSEYLNVESIQEMFQLDYYNMLLTQKQIDVYNAIIGGKTDDEHDVKIKGINDYINLYNQKHKDDKLPKLKALFKQILSDRNAISWLPEEFNSDQEVLNAIKDCYERLSENVLGDKVLKSMLGSLADYSLDGIFIRNDLQLTDISQKMFGNWSVIQNAIMQNIKHVAPARKHKESEEEYENRIAGIFKKADSFSISYIDACLNETDPNNAYFVENYFATLGAVDTPTMQRENLFALVQNAYTEITALLHSDYPTEKNLAQDKANVAKIKALLDAIKSLQHFVKPLLGKGDESDKDERFYGELASLWAELDTMTPLYNMIRNYMTRKPYSQKKIKLNFENPQLLGGWDANKEKDYATIILRRNGLYYLAIMNKDSKKLLGKAMPSDGECYEKMVYKLLPGANKMLPKVFFAKSRMEDFKPSKELVEKYYNGTHKKGKNFNIQDCHNLIDYFKQSIDKHEDWSKFGFKFSDTSTYEDLSGFYREVEQQGYKLSFARVSVSYINQLVEEGKMYLFQIYNKDFSEYSKGTPNMHTLYWKALFDERNLADVVYKLNGQAEMFYRKKSIENTHPTHPANHPILNKNKDNKKKESLFGYDLIKDRRYTVDKFLFHVPITMNFKSSGSENINQDVKAYLRHADDMHIIGIDRGERHLLYLVVIDLQGNIKEQFSLNEIVNDYNGNTYHTNYHDLLDVREDERLKARQSWQTIENIKELKEGYLSQVIHKITQLMVKYHAIVVLEDLNMGFMRGRQKVEKQVYQKFEKMLIEKLNYLVDKKADASVSGGLLNAYQLTSKFDSFQKLGKQSGFLFYIPAWNTSKIDPVTGFVNLLDTRYQNVEKAKSFFSKFDAIRYNKDKEWFEFNLDYDKFGKKAEGTRTKWTLCTRGMRIDTFRNKEKNSQWDNQEVDLTAEMKSLLEHYYIDIHSNLKDAISAQTDKAFFTGLLHILKLTLQMRNSITGTETDYLVSPVVDENGIFYDSRSCGDELPENADANGAYNIARKGLMMIEQIKDAKDLDNLKFDISNKAWLNFAQQKPYKNG

SEQ ID NO: 15 - protein, 1251 aa, synthetic construct
  source 1..1251: note = ART15
MLFQDFTHLYPLSKTVRFELKPIGRTLEHIHAKNFLSQDETMADMYQKVKVILDDYHRDFIADMMGEVKLTKLAEFYDVYLKFRKNPKDDELQKQLKDLQAVLRKESVKPIGNGGKYKAGHDRLFGAKLFKDGKELGDLAKFVIAQEGKSSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNRIIGEVNGYTNKHNQICHKSERIAKLRPLHKQILSDGMGVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIKHHTARHDDESVQAGKLGQYFKHGLAGVDNPIQKIHNNHSTIKGFLERERPAGERALPKIKSGKNPEMTQLRQLKELLDNALNVAHFAKLLMTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKNVYQKMIYKLLPGPNKMLPRVFFAKSNLDYYNPSAELLDKYAQGTHKKGDNFNLKDCHALIDFFKAGINKHPEWQNFGFKFSPTSSYRDLSDFYREVEPQGYQVKFVDINADYIDELVEQGQLYLFQIYNKDFSPKAHGKPNLHTLYFRALFSEDNLANPIYKLNGEAQIFYRKASLGMNETTIHRAGEILENKNPDNPKERVFTYDIIKDRRYTQDKFMLHVPITMNFGVQGMTIKEFNKKVNQSIRQYDDVNVIGIDRGERHLLYLTVINSKGEILEQRSLNDITTASANGTQMTTPYHKILDKREIERLNARVGWGEIETIKELKSGYLSHVVHQVSQLMLKYNAIVVLEDLNFGFKRGRFKVEKQIYQNFENALIKKLNHLELKDKADDEIGSYKNALQLTNNFTDLKNIGKQTGFLFYVPAWNTSKIDPETGFVDLLKPRYENIAQSQAFFGKFDKICYNADKDYFEFHIDYAKFTDKAKNSRQTWTICSHGDKRYVYDKTANQNKGATKGINVNDELKSLFARYHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 16 - protein, 1251 aa, synthetic construct
  source 1..1251: note = ART16
MLFQDFTHLYPLSKTVRFELKPIGKTLEHIHAKNFLSQDETMADMYQKVKAILDDYHRDFITKMMSEVTLTKLPEFYEVYLALRKNPKDDTLQKQLTEIQTALREEVVKPIDSGGKYKAGYERLFGAKLFKDGKELGDLAKFVIAQEGESSPKLPQIAHFEKFSTYFTGFHDNRKNMYSSDDKHTAIAYRLIHENLPRFIDNLQILVTIKQKHSVLYDQIVNELNANGLDVSLASHLDGYHKLLTQEGITAYNRIIGEVNSYTNKHNQICHKSERIAKLRPLHKQILSDGMGVSFLPSKFADDSEMCQAVNEFYRHYAHVFAKVQSLFDRFDDYQKDGIYVEHKNLNELSKQAFGDFALLGRVLDGYYVDVVNPEFNDKFAKAKTDNAKEKLTKEKDKFIKGVHSLASLEQAIEHYIAGHDDESVQAGKLGQYFKHGLAGVDNPIQKIHNSHSTIKGFLERERPAGERTLPKIKSDKSLEMTQLRQLKELLDNALNVVHFAKLLTTKTTLDNQDGNFYGEFGALYDELAKIATLYNKVRDYLSQKPFSTEKYKLNFGNPTLLNGWDLNKEKDNFGVILQKDGCYYLALLDKAHKKVFDNAPNTGKSVYQKMVYKLLPGPNKMLPKVFFAKSNLDYYNPSAELLDKYAQGTHKKGDNFNLKDCHALIDFFKASINKHPEWQHFGFEFSLTSSYQDLSDFYREVEPQGYQVKFVDIDADYIDELVEQGQLYLFQIYNKDFSPKAHGKPNLHTLYFKALFSEDNLANPIYKLNGEAEIFYRKASLDMNETTIHRAGEVLENKNPDNPKERQFVYDIIKDKRYTQDKFMLHVPITMNFGVQGMTIKEFNKKVNQSIQQYDEVNVIGIDRGERHLLYLTVINSKGEILEQRSLNDIITTSANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADNEIGSYKNALQLTNNFTDLKSIGKQTGFLFYVPAWNTSKIDPVTGFVDLLKPRYENIAQSQAFFDKFDKICYNADKGYFEFHIDYAKFTDKAKNSRQIWTICSHGDKRYVYDKTANQNKGATIGINVNDELKSLFARYRINDKQPNLVMDICQNNDKEFHKSLTYLLKALLALRYSNASSDEDFILSPVANDKGVFFNSALADDTQPQNADANGAYHIALKGLWLLNELKNSDDLDKVKLAIDNQTWLNFAQNR

SEQ ID NO: 17 - protein, 1251 aa, synthetic construct
  source 1..1251: note = ART17
MLFQDFTHLYPLSKTVRFELKPIGKTLEHIHAKNFLSQDETMADMYQKVKAILDDYHRDFITKMMSEVTLTKLPEFYEVYLALRKNPKDDTLQKQLTEIQTALREEVVKPIDSGGKYKAGYERLFGAKLFKDGKELGDLAKFVIAQEGESSPKLPQIAHFEKFSTYFTGFHDNRKNMYSSDDKHTAIAYRLIHENLPRFIDNLQILVTIKQKHSVLYDQIVNELNANGLDVSLASHLDGYHKLLTQEGITAYNRIIGEVNSYTNKHNQICHKSERIAKLRPLHKQILSDGMGVSFLPSKFADDSEMCQAVNEFYRHYAHVFAKVQSLFDRFDDYQKDGIYVEHKNLNELSKQAFGDFALLGRVLDGYYVDVVNPEFNDKFAKAKTDNAKEKLTKEKDKFIKGVHSLASLEQAIEHYIAGHDDESVQAGKLGQYFKHGLAGVDNPIQKIHNSHSTIKGFLERERPAGERTLPKIKSDKSLEMTQLRQLKELLDNALNVVHFAKLLTTKTTLDNQDGNFYGEFGALYDELAKIATLYNKVRDYLSQKPFSTEKYKLNFGNPTLLNGWDLNKEKDNFGVILQKDGCYYLALLDKAHKKVFDNAPNTGKSVYQKMVYKLLPGSNKMLPKVFFAKSNLDYYNPSAELLDKYAQGTHKKGDNFNLKDCHALIDFFKASINKHPEWQHFGFEFSLTSSYQDLSDFYREVEPQGYQVKFVDIDADYIDELVEQGQLYLFQIYNKDFSPKAHGKPNLHTLYFKALFSEDNLANPIYKLNGEAEIFYRKASLDMNETTIHRAGEVLENKNPDNPKERQFVYDIIKDKRYTQDKFMLHVPITMNFGVQGMTIKEFNKKVNQSIQQYDEVNVIGIDRGERHLLYLTVINSKGEILEQRSLNDIITTSANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADNEIGSYKNALQLTNNFTDLKSIGKQTGFLFYVPAWNTSKIDPVTGFVDLLKPRYENIAQSQAFFDKFDKICYNADKGYFEFHIDYAKFTDKAKNSRQIWTICSHGDKRYVYDKTANQNKGATIGINVNDELKSLFARYRINDKQPNLVMDICQNNDKEFHKSLTYLLKALLALRYSNASSDEDFILSPVANDKGVFFNSALADDTQPQNADANGAYHIALKGLWLLNELKNSDDLDKVKLAIDNQTWLNFAQNR

SEQ ID NO: 18 - protein, 1257 aa, synthetic construct
  source 1..1257: note = ART18
MKYTDFTGIYPVSKTLRFELIPQGSTVENMKREGILNNDMHRADSYKEMKKLIDEYHKVFIERCLSDFSLKYDDTGKHDSLEEYFFYYEQKRNDKTKKIFEDIQVALRKQISKRFTGDTAFKRLFKKELIKEDLPSFVKNDPVKTELIKEFSDFTTYFQEFHKNRKNMYTSDAKSTAIAYRIINENLPKFIDNINAFHIVAKVPEMQEHFKTIADELRSHLQVGDDIDKMFNLQFFNKVLTQSQLAVYNAVIGGKSEGNKKIQGINEYVNLYNQQHKKARLPMLKLLYKQILSDRVAISWLQDEFDNDQDMLDTIEAFYNKLDSNETGVLGEGKLKQILMGLDGYNLDGVFLRNDLQLSEVSQRLCGGWNIIKDAMISDLKRSVQKKKKETGADFEERVSKLFSAQNSFSIAYINQCLGQAGIRCKIQDYFACLGAKEGENEAETTPDIFDQIAEAYHGAAPILNARPSSHNLAQDIEKVKAIKALLDALKRLQRFVKPLLGRGDEGDKDSFFYGDFMPIWEVLDQLTPLYNKVRNRMTRKPYSQEKIKLNFENSTLLNGWDLNKEHDNTSVILRREGLYYLGIMNKNYNKIFDANNVETIGDCYEKMIYKLLPGPNKMLPKVFFSKSRVQEFSPSKKILEIWESKSFKKGDNFNLDDCHALIDFYKDSIAKHPDWNKFNFKFSDTQSYTNISDFYRDVNQQGYSLSFTKVSVDYVNRMVDEGKLYLFQIYNKDFSPQSKGTPNMHTLYWRMLFDERNLHNVIYKLNGEAEVFYRKASLRCDRPTHPAHQPITCKNENDSKRVCVFDYDIIKNRRYTVDKFMFHVPITINYKCTGSDNINQQVCDYLRSAGDDTHIIGIDRGERNLLYLVIIDQHGTIKEQFSLNEIVNEYKGNTYCTNYHTLLEEKEAGNKKARQDWQTIESIKELKEGYLSQVIHKISMLMQRYHAIVVLEDLNGSFMRSRQKVEKQVYQKFEHMLINKLNYLVNKQYDAAEPGGLLHALQLTSRMDSFKKLGKQSGFLFYIPAWNTSKIDPVTGFVNLFDTRYCNEAKAKEFFEKFDDISYNDERDWFEFSFDYRHFTNKPTGTRTQWTLCTQGTRVRTFRNPEKSNHWDNEEFDLTQAFKDLFNKYGIDIASGLKARIVNGQLTKETSAVKDFYESLLKLLKLTLQMRNSVTGTDIDYLVSPVADKDGIFFDSRTCGSLLPANADANGAFNIARKGLMLLRQIQQSSIDAEKIQLAPIKNEDWLEFAQEKPYL

SEQ ID NO: 19 - protein, 1245 aa, synthetic construct
  source 1..1245: note = ART19
METFSGFTNLYPLSKTLRFRLIPVGETLKYFIGSGILEEDQHRAESYVKVKAIIDDYHRAYIENSLSGFELPLESTGKFNSLEEYYLYHNIRNKTEEIQNLSSKVRTNLRKQVVAQLTKNEIFKRIDKKELIQSDLIDFVKNEPDANEKIALISEFRNFTVYFKGFHENRRNMYSDEEKSTSIAFRLIHENLPKFIDNMEVFAKIQNTSISENFDAIQKELCPELVTLCEMFKLGYFNKTLSQKQIDAYNTVIGGKTTSEGKKIKGLNEYINLYNQQHKQEKLPKMKLLFKQILSDRESASWLPEKFENDSQVVGAIVNFWNTIHDTVLAEGGLKTIIASLGSYGLEGIFLKNDLQLTDISQKATGSWGKISSEIKQKIEVMNPQKKKESYETYQERIDKIFKSYKSFSLAFINECLRGEYKIEDYFLKLGAVNSSSLQKENHFSHILNTYTDVKEVIGFYSESTDTKLIRDNGSIQKIKLFLDAVKDLQAYVKPLLGNGDETGKDERFYGDLIEYWSLLDLITPLYNMVRNYVTQKPYSVDKIKINFQNPTLLNGWDLNKETDNTSVILRRDGKYYLAIMNNKSRKVFLKYPSGTDRNCYEKMEYKLLPGANKMLPKVFFSKSRINEFMPNERLLSNYEKGTHKKSGTCFSLDDCHTLIDFFKKSLDKHEDWKNFGFKFSDTSTYEDMSGFYKEVENQGYKLSFKPIDATYVDQLVDEGKIFLFQIYNKDFSEHSKGTPNMHTLYWKMLFDETNLGDVVYKLNGEAEVFFRKASINVSHPTHPANIPIKKKNLKHKDEERILKYDLIKDKRYTVDQFQFHVPITMNFKADGNGNINQKAIDYLRSASDTHIIGIDRGERNLLYLVVIDGNGKICEQFSLNEIEVEYNGEKYSTNYHDLLNVKENERKQARQSWQSIANIKDLKEGYLSQVIHKISELMVKYNAIVVLEDLNAGFMRGRQKVEKQVYQKFEKKLIEKLNYLVFKKQSSDLPGGLMHAYQLANKFESFNTLGKQSGFLFYIPAWNTSKMDPVTGFVNLFDVKYESVDKAKSFFSKFDSIRYNVERDMFEWKFNYGEFTKKAEGTKTDWTVCSYGNRIITFRNPDKNSQWDNKEINLTENIKLLFERFGIDLSSNLKDEIMQRTEKEFFIELISLFKLVLQMRNSWTGTDIDYLVSPVCNENGEFFDSRNVDETLPQNADANGAYNIARKGMILLDKIKKSNGEKKLALSITNREWLSFAQGCCKNG

SEQ ID NO: 20 - protein, 1245 aa, synthetic construct
  source 1..1245: note = ART20
METFSGFTNLYPLSKTLRFRLIPVGETLKHFIDSGILEEDQHRAESYVKVKAIIDDYHRAYIENSLSGFELPLESTGKFNSLEEYYLYHNIRNKTEEIQNLSSKVRTNLRKQVVVQLTKNEIFKRIDKKELIQSDLIDFVKNEPDANEKIALISEFRNFTVYFKGFHENRRNMYSDEEKSTSIAFRLIHENLPKFIDNMEVFAKIQNTSISENFDAIQKELCPELVTLCEMFKLGYFNKTLSQKQIDAYNTVIGGKTTSEGKKIKGLNEYINLYNQQHKQEKLPKMKLLFKQILSDRESASWLPEKFENDSQVVGAMVNFWNTIHDTVLAEGGLKTIIASLGSYGLEGIFLKNDLQLTDISQKATGSWSKISSEIKQKIEVMNPQKKKESYESYQERIDKLFKSYKSFSLAFINECLRGEYKIEDYFLKLGAVNSSSLQKENHFSHILNAYTDVKEAIGFYSESTDTKLIQDNDSIQKIKQFLDAVKDLQAYVKPLLGNGDETGKDERFYGDLIEYWSLLDLITPLYNMVRNYVTQKPYSVDKIKINFQNPTLLNGWDLNKETDNTSVILRRDGKYYLAIMNNKSRKVFLKYPSGTDGNCYEKMEYKLLPGANKMLPKVFFSKSRINEFMPNERLLSNYEKGTHKKSGICFSLDDCHTLIDFFKKSLDKHEDWKNFGFKFSDTSTYEDMSGFYKEVENQGYKLSFKPIDATYVDQLVDEGKIFLFQIYNKDFSEHSKGTPNMHTLYWKMLFDETNLGDVVYKLNGEAEVFFRKASINVSHPTHPANIPIKKKNLKHKDEERILKYDLIKDKRYTVDQFQFHVPITMNFKADGNGNINQKAIDYLCSASDTHIIGIDRGERNLLYLVVIDGNGKICEQFSLNEIEVEYNGEKYSTNYHDLLNVKENERKQARQSWQSIANIKDLKEGYLSQVIHKISELMVKYNAIVVLEDLNAGFMRGRQKVEKQVYQKFEKKLIEKLNYLVFKKQSSDLPGGLMHAYQLANKFESFNALGKQSGFLFYIPAWNTSKMDPVTGFVNLFDVKYESVDKAKSFFSKFDSMRYNVERDMFEWKFNYGEFTKKAEGTKTDWTVCSYGNRIITFRNPDKNSQWDNKEINLTENIKLLFERFGIDLSSNLKDEIMQRTEKEFFIELISLFKLVLQMRNSWTGTDIDYLVSPVCNENGEFFDSRNVDETLPQNADANGAYNIARKGMILLDKIKKSNGEKKLALSITNREWLSFAQGCCKNG

SEQ ID NO: 21 - protein, 1245 aa, synthetic construct
  source 1..1245: note = ART21
METFSGFTNLYPLSKTLRFRLIPVGETLKHFIGSGILEEDQHRAESYVKVKAIIDDYHRTYIENSLSGFELPLESTGKFNSLEEYYLYHNIRNKTEEIQNLSSKVRTNLRKQVVTQLTKNEIFKRIDKKELIQSDLIDFVKNEPDANEKIALISEFRNFTVYFKGFHENRRNMYSDEEKSTSIAFRLIHENLPKFIDNMEVFAKIQNTSISENFDAIQKELCPELVTLCEMFKLGYFNKTLSQKQIDAYNTVIGGKTTSEGKKIKGLNEYINLYNQQHKQEKLPKMKLLFKQILSDRESASWLLEKFENDSQVVGAMVNFWNTIHDTVLAEGGLKTIIASLGSYGLEGIFLKNDLQLTDISQKATGSWSKISSEIKQKIEAMNPQKKKESYESYQERIDKLFKSYKSFSLAFVNECLRGEYKIEDYFLKLGAVNSSLLQKENHFSHILNTYTDVKEVIGFYSESTDTKLIQDNDSIQKIKQFLDAVKDLQAYVKPLLGNSDETGKDERFYGDLIEYWSLLDLITPLYNMVRNYVTQKPYSVDKIKINFQNPTLLNGWDLNKEMDNTSVILRRDGKYYLAIMNNKSRKVFLKYPSGTDRNCYEKMEYKLLPGANKMLPKVFFSKSRINEFMPNERLLSNYEKGTHKKSGTCFSLDDCHTLIDFFKKSLNKHEDWKNFGFKFSDTSTYEDMSGFYKEVENQGYKLSFKPIDATYVDQLVDEGKIFLFQIYNKDFSEHSKGTPNMHTLYWKMLFDETNLGDVVYKLNGEAEVFFRKASINVSHPTHPANIPIKKKNLKHKDEERILKYDLIKDKRYTVDQFQFHVPITMNFKANGNGNINQKAIDYLRSASDTHIIGIDRGERNLLYLVVIDGNGKICEQFSLNEIEVEYNGEKYSTNYHDLLNVKENERKQARQSWQSIANIKDLKEGYLSQVIHKISELMVKYNAIVVLEDLNAGFMRGRQKVEKQVYQKFEKKLIEKLNYLVFKKQSSDLPGGLMHAYQLANKFESFNTLGKQSGFLFYIPAWNTSKMDPVTGFVNLFDVKYESVDKAKSFFSKFDSIRYNVERDMFEWKFNYDEFTKKAEGTKTDWTVCSYGNRIITFRNPDKNSQWDNKEINLTENIKLLFERFGIDLSSNLKDEIMERTEKEFFIELISLFKLVLQMRNSWTGTDIDYLVSPVCNENGEFFDSRNVDETLPQNADANGAYNIARKGMILLDKIKKNNGEKKLTLSITNREWLSFAQGCCKNG

SEQ ID NO: 22 - protein, 1261 aa, synthetic construct
  source 1..1261: note = ART22
MLFQDFTHLYPLSKTVRFELKPIGKTLEHIHAKNFLSQDKTMADMYQKVKAILDDYHRDFIADMMGEVKLTKLAEFCDVYLKFRKNPKDDGLQKQLKDLQAVLRKEIVKPIGNGGKYKVGYDRLFGAKLFKDGKELGDLAKFVIAQESESSPKLPQIAHFEKFSTYFTGFHDNRKNMYSSDDKHTAIAYRLIHENLPRFIDNLQILATIKQKHSALYDQIASELTASGLDVSLASHLGGYHKLLTQEGITAYNRIIGEVNSYTNKHNQICHKSERIAKLRPLHKQILSDGMGVSFLPSKFADDSEMCQAVNEFYRHYADVFAKVQSLFDRFDDYQKDGIYVEHKNLNELSKRAFGDFGFLKRFLEEYYADVIDPEFNEKFAKTEPDSDEQKKLAGEKDKFVKGVHSLASLEQVIEYYTAGYDDESVQADKLGQYFKHRLAGVDNPIQKIHNSHSTIKGFLERERPAGERALPKIKSDKSPEMTQLRQLKELLDNALNVVHFAKLVSTETVLDTRSDKFYGEFRPLYVELAKITTLYNKVRDYLSQKPFSTEKYKLNFGNPTLLNGWDLNKEKDNFGVILQKDGCYYLALLDKAHKKVFDNAPNTGKSVYQKMVYKQIANARRDLACLLIINGKVVRKTKGLDDLREKYLPYDIYKIYQSESYKVLSPNFNHQDLVKYIDYNKILASGYFEYFDFRFKESSEYKSYKEFLDDVDNCGYKISFCNINADYIDELVEQGQLYLFQIYNKDFSPKAHGKPNLHTLYFKALFSEDNLANPIYKLNGEAQIFYRKASLDMNETTIHRAGEVLENKNPDNPKQRQFVYDIIKDKRYTQDKFMLHVPITMNFGVQGMTIEGFNKKVNQSIQQYDDVNVIGIDRGERHLLYLTVINSKGEILEQRSLNDIITTSANGTQMTTPYHKILNKKKEGRLQARKDWGEIETIKELKAGYLSHVVHQISQLMLKYNAIVVLEDLNFGFKRGRLKVENQVYQNFENALIKKLNHLVLKDKTDDEIGSYKNALQLTNNFTDLKSIGKQTGFLFYVPARNTSKIDPETGFVDLLKPRYENITQSQAFFGKFDKICYNTDKGYFEFHIDYAKFTDEAKNSRQTWVICSHGDKRYVYNKTANQNKGATKGINVNDELKSLFACHHINDKQPNLVMDICQNNDKEFHKSLMYLLKALLALRYSNANSDEDFILSPVANDEGVFFNSALADDTQPQNADANGAYHIALKGLWVLEQIKNSDDLDKVDLEIKDDEWRNFAQNR

SEQ ID NO: 23 - protein, 1275 aa, synthetic construct
  source 1..1275: note = ART23
MGKNQNFQEFIGVSPLQKTLRNELIPTETTKKNITQLDLLTEDEIRAQNREKLKEMMDDYYRDVIDSTLHAGIAVDWSYLFSCMRNHLRENSKESKRELERTQDSIRSQIYNKFAERADFKDMFGASIITKLLPTYIKQNPEYSERYDESMEILKLYGKFTTSLTDYFETRKNIFSKEKISSAVGYRIVEENAEIFLQNQNAYDRICKIAGLDLHGLDNEITAYVDGKTLKEVCSDEGFAKAITQEGIDRYNEAIGAVNQYMNLLCQKNKALKPGQFKMKRLHKQILCKGTTSFDIPKKFENDKQVYDAVNSFTEIVMKNNDLKRLLNITQNVNDYDMNKIYVAADAYSTISQFISKKWNLIEECLLDYYSDNLPGKGNAKENKVKKAVKEETYRSVSQLNELIEKYYVEKTGQSVWKVESYISRLAETITLELCHEIENDEKHNLIEDDDKISKIKELLDMYMDAFHIIKVFRVNEVLNFDETFYSEMDEIYQDMQEIVPLYNHVRNYVTQKPYKQEKYRLYFNTPTLANGWSKNKEYDNNAIILMRDDKYYLGILNAKKKPSKQTMAGKEDCLEHAYAKMNYYLLPGANKMLPKVFLSKKGIQDYHPSSYIVEGYNEKKHIKGSKNFDIRFCRDLIDYFKECIKKHPDWNKFNFEFSATETYEDISVFYREVEKQGYRVEWTYINSEDIQKLEEDGQLFLFQIYNKDFAVGSTGKPNLHTLYLKNLFSEENLRDIVLKLNGEAEIFFRKSSVQKPVIHKCGSILVNRTYEITESGTTRVQSIPESEYMELYRYFNSEKQIELSDEAKKYLDKVQCNKAKTDIVKDYRYTMDKFFIHLPITINFKVDKGNNVNAIAQQYIAEQEDLHVIGIDRGERNLIYVSVIDMYGRILEQKSFNLVEQVSSQGTKRYYDYKEKLQNREEERDKARKSWKTIGKIKELKEGYLSSVIHEIAQMVVKYNAIIAMEDLNYGFKRGRFKVERQVYQKFETMLISKLNYLADKSQAVDEPGGILRGYQMTYVPDNIKNVGRQCGIIFYVPAAYTSKIDPTTGFINAFKRDVVSTNDAKENFLMKFDSIQYDIEKGLFKFSFDYKNFATHKLTLAKTKWDVYTNGTRIQNMKVEGHWLSMEVELTTKMKELLDDSHIPYEEGQNILDDLREMKDITTIVNGILEIFWLTVQLRNSRIDNPDYDRIISPVLNNDGEFFDSDEYNSYIDAQKAPLPIDADANGAFCIALKGMYTANQIKENWVEGEKLPADCLKIEHASWLAFMQGERG

SEQ ID NO: 24 - protein, 1261 aa, synthetic construct
  source 1..1261: note = ART24
MNTSLFSSFTRQYPVTKTLRFELKPMGATLGHIQQKGFLHKDEELAKIYKKIKELLDEYHRAFIADTLGDAQLVGLDDFYADYQALKQDSKNSHLKDKLTKTQDNLRKQITKNFEKTPQLKERYKRLFTKELFKAGKDKGDLEKWLINHDSEPNKAEKISWIHQFENFTTYFQGFYENRKNMYSDEVKHTAIAYRLIHENLPRFVDNIQVLSKIKSDYPDLYHELNHLDSRTIDFADFKFDDMLQMDFYHHLLIQSGITAYNTLLGGKVLEGGKKLQGINELINLYGQKHKIKIAKLKPLHKQILSDGQSVSFLPKKFDNDYELCQTVNHFYREYVAIFDELVVLFQKFYDYDKDNIYINHQQLNQLSHELFADFRLLSRALDFYYCQIIDGDFNNKINNAKSQNAKEKLLKEKERYTKSNHSINELQKAINHYASHHEDTEVKVISDYFSATNIRNMIDGIHHHFSTIKGFLEKDNNQGESYLPKQKNSNDVKNLKLFLDGVLRLIHFIKPLALKSDDTLEKEEHFYGEFMPLYDKLVMFTLLYNKVRDYISQKPYNDEKIKLNFGNSTLLNGWDVNKEKDNFGVILCKEGLYYLAILDKSHKKVFDNAPKATSSHTYQKMVYKLLPGPNKMLPKVFFAKSNIGYYQPSAQLLENYEKGTHKKGSNFSLTDCHHLIDFFKSSIAKHPEWKEFGFRFSDTHTYQDLSDFYKEIEPQSYKVKFIDIDADYIDDLVEKGQLYLFQLYNKDFSKQSYGKPNLHTLYFKSLFSDDNLKNPIYKLNGEAEIFYRRASLSVSDTTIHQAGEILTPKNPNNTHNRTLSYDVIKNKRYTTDKFFLHIPITMNFGIENTGFKAFNHQVNTTLKNADKKDVHIIGIDRGERHLLYVSVIDGDGRIVEQRTLNDIVSISNNGMSMSTPYHQILDNREKERLAARTDWGDIKNIKELKAGYLSHVVHEVVQMMLKYNAMIVLEDLNFGFKHGRFKVEKQVYQNFENALIKKLNYLVLKNADNHQLGSVRKALQLTNNFTDIKSIGKQTGFIFYVPAWNTSKIDPTTGFVDLLKPRYENMAQAQSFISRFKKIAYNHQLDYFEFEFDYADFYQKTIDKKRIWTLCTYGDVRYYYDHKTKETKTVNITKELKSLLDKHDLSYQNGHNLVDELANSHDKSLLSGVMYLLKVLLALRYSHAQKNEDFILSPVMNKDGVFFDSRFADDVLPNNADANGAYHIALKGLWVLNQIQSADNMDKIDLSISNEQWLHFTQSR

SEQ ID NO: 25 - protein, 1288 aa, synthetic construct
  source 1..1288: note = ART25
MVGNKISNSFDSFTGINALSKTLRNELIPSDYTKRHIAESDFIAADTNKNEDQYVAKEMMDDYYRDFISKVLDNLHDIEWKNLFELMHKAKIDKSDATSKELIKIQDMLRKKIGKKFSQDPEYKVMLSAGMITKILPKYILEKYETDREDRLEAIKRFYGFTVYFKEFWASRQNVFSDKAIASSISYRIIHENAKIYMDNLDAYNRIKQIACEEIEKIEEEAYDFLQGDQLDVVYTEEAYGRFISQSGIDLYNNICGVINAHMNLYCQSKKCSRSKFKMQKLHKQILCKAETGFEIPLGFQDDAQVINAINSFNALIKEKNIISRLRTIGKSISLYDVNKIYISSKAFENVSVYIDHKWDVIASSLYKYFSEIVKGNKDNREEKIQKEIKKVKSCSLGDLQRLVNSYYKIDSTCLEHEVTEFVTKIIDEIDNFQITDFKFNDKISLIQNEQIVMDIKTYLDKYMSIYHWMKSFVIDELVDKDMEFYSELDELNEDMSEIVNLYNKVRNYVTQKPYSQEKIKLNFGSPTLADGWSKSKEFDNNAIILIRDEKIYLAIFNPRNKPAKTVISGHDVCNSETDYKKMNYYLLPGASKTLPHVFIKSRLWNESHGIPDEILRGYELGKHLKSSVNFDVEFCWKLIDYYKECISCYPNYKAYNFKFADTESYNDISEFYREVECQGYKIDWTYISSEDVEQLDRDGQIYLFQIYNKDFAPNSKGMDNLHTKYLKNIFSEDNLKNIVIKLNGEAELFYRKSSVKKKVEHKKGTILVNKTYKVEDNTENSKEKRVIIESVPDDCYMELVDYWRNGGIGILSDKAVQYKDKVSHYEATMDIVKDRRYTVDKFFIHLPITINFKADGRININEKVLKYIAENDELHVIGIDRGERNLLYVSVINKKGKIVEQKSFNMIESYETVTNIVRRYNYKDKLVNKESARTDARKNWKEIGKIKEIKEGYLSQVIHEISKMVLKYNAIIVMEDLNYGFKRGRFRVERQVYQKFENMLISKLAYLVDKSRKADEPGGVLRGYQLTYIPDSLEKLGSQCGIIFYVPAAYTSKIDPLTGFVNVFNFREYSNFETKLDFVRSLDSIRYDTEKKLFSISFDYDNFKTHNTTLAKTKWVIYLRGERIKKEHTSYGWKDDVWNVESRIKDLFDSSHMKYDDGHNLIEDILELESSVQKKLINELIEIIRLTVQLRNSKSERYDRTEAEYDRIVSPVMDENGRFYDSENYIFNEETELPKDADANGAYCIALKGLYNVIAIKNNWKEGEKFNRKLLSLNNYNWFDFIQNRRF

SEQ ID NO: 26 - protein, 1876 aa, synthetic construct
  source 1..1876: note = ART26
MVGNKISNSFDSFTGINALSKTLRNELIPSDYTKRHIAESDFIAADTNKNEDQYVAKEMMDDYYRDFISKVLDNLHDIEWKNLFELMHKAKIDKSDATSKELIKIQDMLRKKIGKKFSQDPEYKVMLSAGMITKILPKYILEKYETDREDRLEAIKRFYGFTVYFKEFWASRQNVFSDKAIASSISYRIIHENAKIYMDNLDAYNRIKQIACEEIEKIEEEAYDFLQGDQLDVVYTEEAYGRFISQSGIDLYNNICGVINAHMNLYCQSKKCSRSKFKMQKLHKQILCKAETGFEIPLGFQDDAQVINAINSFNALIKEKNIISRLRTIGKSISLYDVNKIYISSKAFENVSVYIDHKWDVIASSLYKYFSEIVKGNKDNREEKIQKEIKKVKSCSLGDLQRLVNSYYKIDSTCLEHEVTEFVTKIIDEIDNFQITDFKFNDKISLIQNEQIVMDIKTYLDKYMSIYHWMKSFVIDELVDKDMEFYSELDELNEDMSEIVNLYNKVRNYVTQKPYSQEKIKLNFGSPTLADGWSKSKEFDNNAIILIRDEKIYLAIFNPRNKPAKTVISGHDVCNSETDYKKMNYYLLPGASKTLPHVFIKSRLWNESHGIPDEILRGYELGKHLKSSVNFDVEFCWKLIDYYKECISCYPNYKAYNFKFADTESYNDISEFYREVECQGYKIDWTYISSEDVEQLDRDGQIYLFQIYNKDFAPNSKGMDNLHTKYLKNIFSEDNLKNIVIKLNGEAELFYRKSSVKKKVEHKKGTILVNKTYKVEDNTENSKEKRVIIESVPDDCYMELVDYWRNGGIGILSDKAVQYKDKVSHYEATMDIVKDRRYTVDKFFIHLPITINFKADGRININEKVLKYIAENDELHVIGIDRGERNLLYVSVINKKGKIVEQKSFNMIESYETVTNIVRRYNYKDKLVNKESARTDARKNWKEIGKIKEIKEGYLSQVIHEISKMVLKYNAIIVMEDLNYGFKRGRFRVERQVYQKFENMLISKLAYLVDKSRKADEPGGVLRGYQLTYIPDSLEKLGSQCGIIFYVPAAYTSKIDPLTGFVNVFNFREYSNFETKLDFVRSLDSIRYDTEKKLFSISFDYDNFKTHNTTLAKTKWVIYLRGERIKKEHTSYGWKDDVWNVESRIKDLFDSSHMKYDDGHNLIEDILELESSVQKKLINELIEIIRLTVQLRNSKSERYDRTEAEYDRIVSPVMDENGRFYDSENYIFNEETELPKDADANGAYCIALKGLYNVIAIKNNWKEGEKFNRKLLSLNNYNWFDFIQNRRFQIYLFQIYNKDFAPNSKGMDNLHTKYLKNIFSEDNLKNIVIKLNGEAELFYRKSSVKKKVEHKKGTILVNKTYKVEDNTENSKEKRVIIESVPDDCYMELVDYWRNGGIGILSDKAVQYKDKVSHYEATMDIVKDRRYTVDKFFIHLPITINFKADGRININEKVLKYIAENDELHVIGIDRGERNLLYVSVINKKGKIVEQKSFNMIESYETVTNIVRRYNYKDKLVNKESARTDARKNWKEIGKIKEIKEGYLSQVIHEISKMVLKYNAIIVMEDLNYGFKRGRFRVERQVYQKFENMLISKLAYLVDKSRKADEPGGVLRGYQLTYIPDSLEKLGSQCGIIFYVPAAYTSKIDPLTGFVNVFNFREYSNFETKLDFVRSLDSIRYDTEKRLFSISFDYDNFKTHNTTLAKTKWVIYLRGERIKKEHTSYGWKDDVWNVESRIKDLFDSSHMKYDDGHNLIEDILELESSVQKKLINELIEIIRLTVQLRNSKSERYDRTEAEYDRIVSPVMDEKGRFYDSENYIFNEETELPKDADANGAYCIALKGLYNVIAIKNNWKEGEKFNRKLLSLNNYNWFDFIQNRRF

SEQ ID NO: 27 - protein, 1245 aa, synthetic construct
  source 1..1245: note = ART27
MQEHKKISHLTHRNSVQKTIRMQLNPVGKTMDYFQAKQILENDEKLKEDYQKIKEIADRFYRNLNEDVLSKTGLDKLKDYAEIYYHCNTDADRKRLDECASELRKEIVKNFKNRDEYNKLFNKKMIEIVLPQHLKNEDEKEVVASFKNFTTYFTGFFTNRKNMYSDGEESTAIAYRCINENLPKHLDNVKAFEKAISKLSKNAVDDLDTTYSGLCGTNLYDVFTVDYFNFLLPQSGITEYNKIIGGYTTSDGTKVKGINEYINLYNQQVSKRYKIPNLKILYKQILSESEKVSFIPPKFEDDNELLSAVSEFYANDETFDGMPLKKAIDETKLLFGNLDNSSLNGIYIQNDRSVTNLSNSMFGSWSVIEDLWNKNYDSVNSNSRIKDIQKREDKRKKAYKAEKKLSLSFLQVLISNSENDEIREKSIVDYYKTSLMQLTDNLSDKYKEAAPLFNESYANEKGLKNDDKSISLIKNFLDAIKEIEKFIKPLSETNITGEKNDLFYSQFTPLLDNISRIDILYDKVRNYVTQKPFSTDKIKLNFGNSQLLNGWDRNKEKDCGAVWLCKDEKYYLAIIDKSNNSILENIDFQDCDESDCYEKIIYKLLPGPNKMLPKVFFSEKCKKLLSPSDEILKIRKNGTFKKGDKFSLDDCHKLIDFYKESFKKYPNWLIYNFKFKKTNEYNDISEFYNDVASQGYNISKMKIPTSFIDKLVDEGKIYLFQLYNKDFSPHSKGTPNLHTLYFKMLFDERNLEDVVYKLNGEAEMFYRPASIKYDKPTHPKNTPIKNKNTLNDKRASTFPYDLIKDKRYTKWQFSLHFPITMNFKAPDRAMINDDVRNLLKSCNNNFIIGIDRGERNLLYVSIIDSNGAIIYQHSLNIIGNKFKGKTYETNYREKLETREKERTEQRRNWKAIESIKELKEGYISQAVHVICQLVVKYDAIIVMEKLTDGFKRGRTKFEKQVYQKFEKMLIDKLNYYVDKKLDPDEEGGLLHAYQLTNKLESFDKLGMQSGFIFYVRPDFTSKIDPVTGFVNLLYPRYENIDKAKDMISRFDDIRYNAGEDFFEFDIDYDKFPKTASDYRKKWTICTNGERIEAFRNPASNNEWSYRTIILAEKFKELFDNNSINYRDSDNLKAEILSQTKGKFFEDFFKLLRLTLQMRNSNPETGEDRILSPVKDKNGNFYDSSKYDEKSNLPCDADANGAYNIARKGLWIVEQFKKSDNVSTVEPVIHNDKWLKFVQENDMANN

SEQ ID NO: 28 - protein, 1271 aa, synthetic construct
  source 1..1271: note = ART28
MKNLANFTNLYSLQKTLRFELKPIGKTLDWIIKKDLLKQDEILAEDYKIVKKIIDRYHKDFIDLAFESAYLQKKSSDSFTAIMEASIQSYSELYFIKEKSDRDKKAMEEISGIMRKEIVECFTGKYSEVVKKKFGNLFKKELIKEDLLNFCEPDELPIIQKFADFTTYFTGFHENRENMYSNEEKATAIANRLIRENLPRYLDNLRIIRSIQGRYKDFGWKDLESNLKRIDKNLQYSDFLTENGFVYTFSQKGIDRYNLILGGQSVESGEKIQGLNELINLYRQKNQLDRRQLPNLKELYKQILSDRTRHSFVPEKFSSDKALLRSLLDFHKEVIQNKNLFEEKQVSLLQAIRETLTDLKSFDLDRIYLTNDTSLTQISNFVFGDWSKVKTILAIYFDENIANPKDRQRQSNSYLKAKENWLKKNYYSIHELNEAISVYGKHSDEELPNTKIEDYFSGLQTKDETKKPIDVLDAIVSKYADLESLLTKEYPEDKNLKSDKGSIEKIKNYLDSIKLLQNFLKPLKPKKVQDEKDLGFYNDLELYLESLESANSLYNKVRNYLTGKEYSDEKIKLNFKNSTLLDGWDENKETSNLSVIFRDTNNYYLGILDKQNNRIFESIPEIQSGEETIQKMVYKLLPGANNMLPKVFFSEKGLLKFNPSDEITSLYSEGRFKKGDKFSINSLHTLIDFYKKSLAVHEDWSVFNFKFDETSHYEDISQFYRQVESQGYKITFKPISKKYIDTLVEDGKLYLFQIYNKDFSQNKKGGGKPNLHTIYFKSLFEKENLKDVIVKLNGQAEVFFRKKSIHYDENITRYGHHSELLKGRFSYPILKDKRFTEDKFQFHFPITLNFKSGEIKQFNARVNSYLKHNKDVKIIGIDRGERHLLYLSLIDQDGKILRQESLNLIKNDQNFKAINYQEKLHKKEIERDQARKSWGSIENIKELKEGYLSQVVHTISKLMVEHNAIVVLEDLNFGFKRGRQKVERQVYQKFEKMLIEKLNFLVFKDKEMDEPGGILKAYQLTDNFVSFEKMGKQTGFVFYVPAWNTSKIDPKTGFVNFLHLNYENVNQAKELIGKFDQIRYNQDRDWFEFQVTTDQFFTKENAPDTRTWIICSTPTKRFYSKRTVNGSVSTIEIDVNQKLKELFNDCNYQDGEDLVDRILEKDSKDFFSKLIAYLRILTSLRQNNGEQGFEERDFILSPVVGSDGKFFNSLDASSQEPKDADANGAYHIALKGLMNLHVINETDDESLGKPSWKISNKDWLNFVWQRPSLKA

SEQ ID NO: 29 - protein, 1245 aa, synthetic construct
  source 1..1245: note = ART29
MQEHKKISHLTHRNSVQKTIRMQLNPVGKTMDYFQAKQILENDEKLKENYQKIKEIADRFYRNLNEDVLSKTRLDKLKDYTDIYYHCNTDADRKRLDECASELRKEIVKNFKNRDEYNKLFNKKMIEIVLPKHLKNEDEKEVVTSFKNFTTYFTGFFTNRKNMYSDGEESTAIAYRCINENLPKHLDNVKAFEKAISKLSKNAIDDLDTTYSGLCGTNLYDVFTVDYFNFLLPQSGITEYNKIIGGYTTNDGTKVKGINEYINLYNQQVSKRDKIPNLKILYKQILSESEKVSFIPPKFEDDNELLSAVSEFYANDETFDGMPLKKAIDETKLLFGNLDNPSLNGIYIQNDRSVTNLSNSMFGSWSVIEDLWNKNYDSVNSNSRIKDIQKREDKRKKAYKAEKKLSLSFLQVLISNSENDEIREKSIVDYYKTSLMQLTDNLSDKYNEAAPLLNENYSNEKGLKNDDKSISLIKNFLDAIKEIEKFIKPLSETNITGEKNDLFYSQFTPLLDNISRIDILYDKVRNYVTQKPFSTDKIKLNFGNSQLLNGWDRNKEKDCGAVWLCKDEKYYLAIIDKSNNSILENIDFQDCDESDCYEKIIYKLLPGPNKMLPKVFFSEKCKKLLSPSDEILKIYKSGTFKTGDKFSLDDCHKLIDFYKESFKKYPNWLIYNFKFKKTNEYNDIREFYNDVALQGYNISKMKIPTSFIDKLVDEGKIYLFQLYNKDFSPHSKGTPNLHTLYFKMLFDERNLEDVVYRLNGEAEMFYRPASIKYDKPTHPKNTPIKNKNTLNDKKTSTFPYDLIKDKRYTKWQFSLHFPITMNFKAPDKAMINDDVRNLLKSCNNNFIIGIDRGERNLLYVSVIDSNGAIIYQHSLNIIGNKFKEKTYETNYREKLATREKERTEQRRNWKAIESIKELKEGYISQAVHVICQLVVKYDAIIVMEKLTDGFKRGRTKFEKQVYQKFEKMLIDKLNYYVDKKLDPDEEGGLLHAYQLTNKLESFDKLGMQSGFIFYVRPDFTSKIDPVTGFVNLLYPQYENIDKAKDMISRFDEIRYNAGEDFFEFDIDYDEFPKTASDYRKKWTICTNGERIEAFRNPANNNEWSYRTIILAEKFKELFDNNSINYRDSDDLKAEILSQTKGKFFEDFFKLLRLTLQMRNSNPETGEDRILSPVKDKNGNFYDSSKYDEKSKLPCDADANGAYNIARKGLWIVEQFKKADNVSTVEPVIHNDQWLKFVQENDMANN

SEQ ID NO: 30 - protein, 1245 aa, synthetic construct
  source 1..1245: note = ART30
MQEHKKISHLTHRNSVQKTIRMQLNPVGKTMDYFQAKQILENDEKLKEDYQKIKEIADRFYRNLNEDVLSKTGLDKLKDYADIYYHCNTDADRKRLNECASELRKEIVKNFKNRDEYNKLFNKKMIEIVLPKHLKNEDEKEVVASFKNFTTYFTGFFTNRKNMYSDGEESTAIAYRCINENLPKHLDNVKVFEKAISKLSKNAIDDLGATYSGLCGTNLYDVFTVDYFNFLLPQSGITEYNKIIGGYTTSDGTKVKGINEYINLYNQQVSKRDKIPNLKILYKQILSESEKVSFIPPKFEDDNELLSAVSEFYANDETFDGMPLKKAIDETKLLFGNLDNSSLNGIYIQNDRSVTNLSNSMFGSWSVIEDLWNKNYDSVNSNSRIKDIQKREDKRKKAYKAEKKLSLSFLQVLISNSENDEIREKSIVDYYKTSLMQLTDNLSDKYKEAAPLFSENYDNEKGLKNDDKSISLIKNFLDAIKEIEKFIKPLSETNITGEKNDLFYSQFTPLLDNISRIDILYDKVRNYVTQKPFSTDKIKLNFGNSQLLNGWDKDKEREYGAVLLCKDEKYYLAIIDKSNNSILENIDFQDCNESDYYEKIVYKLLTKINGNLPRVFFSEKRKKLLSPSDEILKIYKSGTFKKGDKFSLDDCHKLIDFYKESFKKYPNWLIYNFKFKNTNEYNDISEFYNDVASQGYNISKMKIPTTFIDKLVDEGKIYLFQLYNKDFSPHSKGTPNLHTLYFKMLFDERNLEDVVYKLNGEAEMFYRPASIKYDKPTHPKNTPIKNKNTLNDKKASTFPYDLIKDKRYTKWQFSLHFPITMNFKAPDKAMINDDVRNLLKSCNNNFIIGIDRGERNLLYVSVIDSNGAIIYQHSLNIIGNKFKGKTYETNYREKLATREKDRTEQRRNWKAIESIKELKEGYISQAVHVICQLVVKYDAIIVMEKLTDGFKRGRTKFEKQVYQKFEKMLIDKLNYYVDKKLDPDEEGGLLHAYQLTNKLESFDKLGTQSGFIFYVRPDFTSKIDPVTGFVNLLYPRYENIDKAKDMISRFDDIRYNAGEDFFEFDIDYDKFPKTASDYRKKWTICTNGERIEAFRNPANNNEWSYRTIILAEKFKELFDNNSINYRDSDDLKAEILSQTKGKFFEDFFKLLRLTLQMRNSNPETGEDRILSPVKDKNGNFYDSSKYDEKSKLPCDADANGAYNIARKGLWIVEQFKKADNVSTVEPVIHNDKWLKFVQENDMANN

SEQ ID NO: 31 - protein, 1244 aa, synthetic construct
  source 1..1244: note = ART31
MQERKKISHLTHRNSVKKTIRMQLNPVGKTMDYFQAKQILENDEKLKENYQKIKEIADRFYRNLNEDVLSKTGLDKLKDYAEIYYHCNTDADRKRLNKCASELRKEIVKNFKNRDEYNKLFDKRMIEIVLPKHLKNEDEKEVVASFKNFTTYFTGFFTNRKNMYSDGEESTAIAYRCINENLPKHLDNVKAFEKAISKLSKNAIDDLDAYSGLCGTNLYDVFTVDYFNFLLPQSGITEYNKIIGGYTTNDGTKVKGINEYINLYNQQVSKRDKIPNLQILYKQILSESEKVSFIPPKFEDDNELLSAVSEFYANDETFDGMPLKKAIDETKLLFGNLDNSSLNGIYIQNDRSVTNLSNSMFGSWSVIEDLWNKNYDSVNSNSRIKDIQKREDKRKKAYKAEKKLSLSFLQVLISNSENDEIRKKSIVDYYKTSLMQLTDNLSDKYNEAAPLLNENYSNEKGLKNDDKSISLIKNFLDAIKEIEKFIKPLSETNITGEKNDLFYSQFTPLLDNISRIDILYDKVRNYVTQKPFSTDKIKLNFGNYQLLNGWDKDKEREYGAVLLCKDEKYYLAIIDKSNNRILENIDFQDCDESDCYEKIIYKLLPTPNKMLPKVFFAKKHKKLLSPSDEILKIYKNGTFKKGDKFSLDDCHKLIDFYKESFKKYPKWLIYNFKFKKTNGYNDIREFYNDVALQGYNISKMKIPTSFIDKLVDEGKIYLFQLYNKDFSPHSKGTPNLHTLYFKMLFDERNLEDVVYRLNGEAEMFYRPASIKYDKPTHPKNTPIKNKNTLNDKRASTFPYDLIKDKRYTKWQFSLHFPITMNFKDPDKAMINDDVRNLLKSCNNNFIIGIDRGERNLLYVSVINSNGAIIYQHSLNIIGNKFKGKTYETNYREKLATREKDRTEQRRNWKAIESIKELKEGYISQAVHVICQLVVKYDAIIVMEKLTDGFKRGRTKFEKQVYQKFEKMLIDKLNYYVDKKLDPDEEGGLLHAYQLTNKLESFDKLGTQSGFIFYVRPDFTSKIDPVTGFVNLLYPRYEKIDKAKDMISRFDDIRYNAGEDFFEFDIDYDKFPKTASDYRKKWTICTNGERIEAFRNPANNNEWSYRTIILAEKFKELFDNNSINYRDSDDLKAEILSQTKGKFFEDFFKLLRLTLQMRNSNPETGEDRILSPVKDKNGNFYDSSKYDEKSKLPCDADANGAYNIARKGLWIVEQFKKADNVSTVEPVIHNDKWLKFVQENDMANN

SEQ ID NO: 32 - protein, 1175 aa, synthetic construct
  source 1..1175: note = ART32
KTGLDKLKDYAEIYYHCNTDADRKRLNKCASELRKEIVKNFKNRDEYNKLFDKRMIEIVLPKHLKNEDEKEVVASFKNFTTYFTGFFTNRKNMYSDGEESTAIAYRCINENLPKHLDNVKAFEKAISKLSKNAIDDLDATYSGLCGTNLYDVFTVDYFNFLLPQSGITEYNKIIGGYTTSDGTKVKGINEYINLYNQQVSKRDKIPNLQILYKQILSESEKVSFIPPKFEDDNELLSAVSEFYANDETFDEMPLKKAIDETKLLFGNLDNSSLNGIYIQNDRSVTNLSNSMFGSWSVIEDLWNKNYDSVNSNSRIKDIQKREDKRKKAYKAEKKLSLSFLQVLISNSENNEIREKSIVDYYKTSLMQLTDNLSDKYNEVAPLLNENYSNEKGLKNDDKSISLIKNFLDAIKEIEKFIKPLSETNITGEKNDLFYSQFTPLLDNISRIDILYDKVRNYVTQKPFSTDKIKLNFGNYQLLNGWDKDKEREYGAVLLCRDEKYYLAIIDKSNNRILENIDFQDCDESDCYEKIIYKLLPTPNKMLPKVFFAKKHKKLLSPSDEILKIRKNGTFKKGDKFSLDDCHKLIDFYKESFKKYPNWLIYNFKFKKTNEYNDIREFYNDVALQGYNISKMKIPTSFIDKLVDEGKIYLFQLYNKDFSPHSKGTPNLHTLYFKMLFDERNLEDVVYKLNGEAKMFYRPASIKYDKPTHPKNTPIKNKNTLNDKKASTFPYDLIKDKRYTKWQFSLHFSITMNFKAPDKAMINDDVRNLLKSCNNNFIIGIDRGERNLLYVSVIDSNGAIIYQHSLNIIGNKFKGKTYETNYREKLATREKERTEQRRNWKAIESIKELKEGYISQAVHVICQLVVKYDAIIVMEKLTDGFKRGRTKFEKQVYQKFEKMLIDKLNYYVDKKLDPDEEGGLLHAYQLTNKLESFDKLGTQSGFIFYVRPDFTSKIDPVTGFVNLLYPRYENIDKAKDMISRFDDIRYNAGEDFFEFDIDYDKFPKTASDYRKKWTICTNGERIEAFRNPANNNEWSYRTIILAEKFKELFDNNSINYRDSDDLKAEILSQTKGKFFEDFFKLLRLTLQMRNSNPETGEDRILSPVKDKNGNFYDSSKYDEKSKLPCDADANGAYNIARKGLWIVEQFKKSDNVSTVEPVIHNDKWLKFVQENDMANN

SEQ ID NO: 33 - protein, 1278 aa, synthetic construct
  source 1..1278: note = ART33
MSININKFSDECRKIDFFTDLYNIQKTLRFSLIPIGATADNFEFKGRLSKEKDLLDSAKRIKEYISKYLADESDICLSQPVKLKHLDEYYELYITKDRDEQKFKSVEEKLRKELADLLKEILKRLNKKILSDYLPEYLEDDEKALEDIANLSSFSTYFNSYYDNCKNMYTDKEQSTAIPYRCINDNLPKFIDNMKAYEKALEELKPSDLEELRNNFKGVYDTTVDDMFTLDYFNCVLSQSGIDSYNAIIGNDKVKGINEYINLHNQTAEQGHKVPNLKRLYKQIGSQKKTISFLPSKFESDNELLKAVYDFYNTGDAEKNFTALKDTITEFEKIFDNLSEYNLDGVFVRNDISLTNLSQSMFNDWSVFRNLWNDQYDKVNNPEKAKDIDKYNDKRHKVYKKSESFSINQLQELIATTLEEDINSKKITDYFSCDFHRVTTEVENKYQLVKDLLSSDYPKNKNLKTSEEDVALIKDFLDSVKSLESFVKILTGTGKESGKDELFYGSFTKWFDQLRYIDKLYDKVRNYITEKPYSLDKIKLSFDNPQFLGGWQHSKETDYSAQLFMKDGLYYLGVMDKETKREFKTQYNTPENDSDTMVKIEYNQIPNPGRVIQNLMLVDGKIVKKNGRKNADGVNAVLEELKNQYLPENINRIRKTESYKTTSNNFNKDDLKAYLEYYIARTKEYYCKYNFVFKSADEYGSFNEFVDDVNNQAYQITKVKVSEKQLLSLVEQGKLYLFKIYNKDFSEYSKGKKNLHTMYFQMLFDDRNLENLVYKLQGGAEMFYRPASIKKDSEFKHDANVEIIKRTCEDKVNDKDNPTDDEKAKYYSKFDYDIVKNKRFTKDQFSLHLTLAMNCNQPDHYWLNNDVRELLKKSNKNHIIGIDRGERNLIYVTIINSDGVIVDQINFNIIENSYNGKKYKTDYQKKLNQREEDRQKARKTWKTIETIKELKDGYISQVVHQICKLIVQYDAIVVMENLNGGFKRGRTKVEKQVYQKFETMLINKLNYYVDKGTDYKECGGLLKAYQLTNKFETFERIGKQSGIIFYVDPYLTSKIDPVTGFANLLYPKYETIPKTHNFISNIDDIRYNQSEDYFEFDIDYDKFPQGSYNYRKKWTICSYGNRIKYYKDSRNKTASVVVDITEKFKETFTNAGIDFVNDNIKEKLLLVNSKELLKSFMDTLKLTVQLRNSEINSDVDYIISPIKDRNGNFYYSENYKKSNNEVPSQPQDGDANGAYNIARKGLMIINKLKKADDVTNNELLKISKKEWLEFAQKGDLGE

SEQ ID NO: 34 - protein, 1562 aa, synthetic construct
  source 1..1562: note = ART34
MKATSIWDNFTRKYSVSKTLRFELRPVGKTEENIVKKEIIDAEWISGKNIPKGTDADRARDYKIVKKLLNQLHILFINQALSSENVKEFEKEDKKSKTFVAWSDLLATHFDNWIQYTRDKSNSTVLKSLEKSKKDLYSKLGKLLNSKANAWKAEFISYHKIKSPDNIKIRLSASNVQILFGNTSDPIQLLKYQIELDNIKFLKDDGSEYTTKELADLLSTFEKFGTYFSGFNQNRANVYDIDGEISTSIAYRLFNQNIEFFFQNIKRWEQFTSSIGHKEAKENLKLVQWDIQSKLKELDMEIVQPRFNLKFEKLLTPQSFIYLLNQEGIDAFNTVLGGIPAEVKAEKKQGVNELINLTRQKLNEDKRKFPSLQIMYKQIMSERKTNFIDQYEDDVEMLKEIQEFSNDWNEKKKRHSASSKEIKESAIAYIQREFHETFDSLEERATVKEDFYLSEKSIQNLSIDIFGGYNTIHNLWYTEVEGMLKSGERPLTRVEKEKLKKQEYISFAQIERLISKHSQQYLDSTPKEANDRSLFKEKWKKTFKNGFKVSEYTNLKLNELISEGETFQKIDQETGKETTIKIPGLFESYENAILVESIKNQSLGTNKKESVPSIKEYLDSCLRLSKFIESFLVNSKDLKEDQSLDGCSDFQNTLTQWLNEEFDVFILYNKVRNHVTKKPGNTDKIKINFDNATLLDGWDVDKEAANFGFLLKKADNYYLGIADSSFNQDLKYFNEGERLDEIEKNRKNLEKEESKNISKIDQEKVKKYKEVIDDLKAISNLNKGRYSKAFYKQSKFTTLIPKCTTQLNEVIEHFKKFDTDYRIENKKFAKPFIITKEVFLLNNTVYDTATKKFTLKIGEDEDTKGLKKFQIGYYRATDDKKGYESALRNWITFCIEFTKSYKSCLNYNYSSLKSVSEYKSLDEFYKDLNGIGYTIDFVDISEEYINKKINEGKLYLFQIYNKDFSEKSKGKENLHTTYWKLLFDSKNLEDVVIKLNGQAEVFFRPASIHEKEKITHFKNQEIQNKNPNAVKKTSKFEYDIIKDNRFTKNKFLFHCPITLNFKADGNPYVNNEVQENIAKNPNVNIIGIDRGEKHLLYFTVINQQGQILDAGSLNSIKSEYKDKNQQSVSFETPYHKILDKKESERKEARESWQEIENIKELKAGYLSHVVHQLSNLIVKYNAIVVLEDLNKGFKRGRFKVEKQVYQKFEKSLIEKLNYLVFKDRKESNEPGHHLNAYQLTNKFLSFERLGKQSGVLFYATASYTSKVDPVTGFMQNIYDPYHKEKTREFYKNFTKIVYNGNYFEFNYDLNSVKPDSEEKRYRTNWTVCSCVIRSEYDSNSKTQKTYNVNDQLVKLFEDAKIKIENGNDLKSTILEQDDKFIRDLHFYFIAIQKMRVVDSKIEKGEDSNDYIQSPVYPFYCSKEIQPNKKGFYELPSNGDSNGAYNIARKGIVILDKIRLRVQIEKLFEDGTKIDWQKLPNLISKVKDKKLLMTVFEEWAELTHQGEVQQGDLLGKKMSKKGEQFAEFIKGLNVTKEDWEIYTQNEKVVQKQIKTWKLFSNST

SEQ ID NO: 35 - protein, 1742 aa, synthetic construct
  source 1..1742: note = ART35
MKAINEYYKQLGAYCREEGKEKDDFFKRIDGAYCAISHLFFGEHGEIAQSDSDVELIQKLLEAYKGLQRFIKPLLGHGDEADKDNEFDAKLRKVWDELDIITPLYDKVRNWLSRKIYNPEKIKLCFENNGKLLSGWVDSRTKSDNGTQYGGYIFRKKNEIGEYDFYLGISADTKLFRRDAAISYDDGMYERLDYYQLKSKTLLGNSYVGDYGLDSMNLLSAFKNAAVKFQFEKEVVPKDKENVPKYLKRLKLDYAGFYQILMNDDKVVDAYKIMKQHILATLTSSIRVPAAIELATQKELGIDELIDEIMNLPSKSFGYFPIVTAAIEEANKRENKPLFLFKMSNKDLSYAATASKGLRKGRGTENLHSMYLKALLGMTQSVFDIGSGMVFFRHQTKGLAETTARHKANEFVANKNKLNDKKKSIFGYEIVKNKRFTVDKYLFKLSMNLNYSQPNNNKIDVNSKVREIISNGGIKNIIGIDRGERNLLYLSLIDLKGNIVMQKSLNILKDDHNAKETDYKGLLTEREGENKEARRNWKKIANIKDLKRGYLSQVVHIISKMMVEYNAIVVLEDLNPGFIRGRQKIERNVYEQFERMLIDKLNFYVDKHKGANETGGLLHALQLTSEFKNFKKSEHQNGCLFYIPAWNTSKIDPATGFVNLFNTKYTNAVEAQEFFSKFDEIRYNEEKDWFEFEFDYDKFTQKAHGTRTKWTLCTYGMRLRSFKNSAKQYNWDSEVVALTEEFKRILGEAGIDIHENLKDAICNLEGKSQKYLEPLMQFMKLLLQLRNSKAGTDEDYILSPVADENGIFYDSRSCGDQLPENADANGAYNIARKGLMLIEQIKNAEDLNNVKFDISNKAWLNFAQQKPYKNGMKAINEYYKQLGAYCREEGKEKDDFFKRIDGAYCAISHLFFGEHGEIAQSDSDVELIQKLLEAYKGLQRFIKPLLGHGDEADKDNEFDAKLRKVWDELDIITPLYDKVRNWLSRKIYNPEKIKLCFENNGKLLSGWVDSRTKSDNGTQYGGYIFRKKNEIGEYDFYLGISADTKLFRRDAAISYDDGMYERLDYYQLKSKTLLGNSYVGDYGLDSMNLLSAFKNAAVKFQFEKEVVPKDKENVPKYLKRLKLDYAGFYQILMNDDKVVDAYKIMKQHILATLTSSIRVPAAIELATQKELGIDELIDEIMNLPSKSFGYFPIVTAAIEEANKRENKPLFLFKMSNKDLSYAATASKGLRKGRGTENLHSMYLKALLGMTQSVFDIGSGMVFFRHQTKGLAETTARHKANEFVANKNKLNDKKKSIFGYEIVKNKRFTVDKYLFKLSMNLNYSQPNNNKIDVNSKVREIISNGGIKNIIGIDRGERNLLYLSLIDLKGNIVMQKSLNILKDDHNAKETDYKGLLTEREGENKEARRNWKKIANIKDLKRGYLSQVVHIISKMMVEYNAIVVLEDLNPGFIRGRQKIERNVYEQFERMLIDKLNFYVDKHKGANETGGLLHALQLTSEFKNFKKSEHQNGCLFYIPAWNTSKIDPATGFVNLFNTKYTNAVEAQEFFSKFDEIRYNEEKDWFEFEFDYDKFTQKAHGTRTKWTLCTYGMRLRSFKNSAKQYNWDSEVVALTEEFKRILGEAGIDIHENLKDAICNLEGKSQKYLEPLMQFMKLLLQLRNSKAGTDEDYILSPVADENGIFYDSRSCGDQLPENADANGAYNIARKGLMLIEQIKNAEDLNNVKFDISNKAWLNFAQQKPYKNG

SEQ ID NO: 36 - protein, 1205 aa, synthetic construct
  source 1..1205: note = ART11*
MYYQGLTKLYPISKTIRNELIPVGKTLEHIRMNNILEADIQRKSDYERVKKLMDDYHKQLINESLQDVHLSYVEEAADLYLNASKDKDIVDKFSKCQDKLRKEIVNLLKSHENFPKIGNKEIIKLLQSLSDTEKDYNALDSFSKFYTYFTSYNEVRKNLYSDEEKSSTAAYRLINENLPKFLDNIKAYSIAKSAGVRAKELTEEEQDCLFMTETFERTLTQDGIDNYNELIGKLNFAINLYNQQNNKLKGFRKVPKMKELYKQILSEREASFVDEFVDDEALLTNVESFSAHIKEFLESDSLSRFAEVLEESGGEMVYIKNDTSKTTFSNIVFGSWNVIDERLAEEYDSANSKKKKDEKYYDKRHKELKKNKSYSVEKIVSLSTETEDVIGKYIEKLQADIIAIKETREVFEKVVLKEHDKNKSLRKNTKAIEAIKSFLDTIKDFERDIKLISGSEHEMEKNLAVYAEQENILSSIRNVDSLYNMSRNYLTQKPFSTEKFKLNFNRATLLNGWDKNKETDNLGILLVKEGKYYLGIMNTKANKSFVNPPKPKTDNVYHKVNYKLLPGPNKMLPKVFFAKSNLEYYKPSEDLLAKYQAGTHKKGENFSLEDCHSLISFFKDSLEKHPDWSEFGFKFSDTKKYDDLSGFYREVEKQGYKITYTDIDVEYIDSLVEKDELYFFQIYNKDFSPYSKGNYNLHTLYLTMLFDERNLRNVVYKLNGEAEVFYRPASIGKDELIIHKSGEEIKNKNPKRAIDKPTSTFEYDIVKDRRYTKDKFMLHIPVTMNFGVDETRRFNEVVNDAIRGDDKVRVIGIDRGERNLLYVVVVDSDGTILEQISLNSIINNEYSIETDYHKLLDEKEGDRDRARKNWTTIENIKELKEGYLSQVVNVIAKLVLKYDAIICLEDLNFGFKRGRQKVEKQVYQKFEKMLIDKLNYLVIDKSRSQENPEEVGHVLNALQLTSKFTSFKELGKQTGIIYYVPAYLTSKIDPTTGFANLFYVKYESVEKSKDFFNRFDSICFNKVAGYFEFSFDYKNFTDRACGMRSKWKVCTNGERIIKYRNEEKNSSFDDKVIVLTEEFKKLFNEYGIAFNDCMDLTDAINAIDDASFFRKLTKLFQQTLQMRNSSADGSRDYIISPVENDNGEFFNSEKCDKSKPKDADANGAFNIARKGLWVLEQLYNSSSGEKLNLAMTNAEWLEYAQQHTI

SEQ ID NO: 37 - protein, 1263 aa, synthetic construct
  source 1..1263: note = MAD7
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 38 - protein, 1334 aa, synthetic construct
  source 1..1334: note = MAD2
MSSLTKFTNKYSKQLTIKNELIPVGKTLENIKENGLIDGDEQLNENYQKAKIIVDDFLRDFINKALNNTQIGNWRELADALNKEDEDNIEKLQDKIRGIIVSKFETFDLFSSYSIKKDEKIIDDDNDVEEEELDLGKKTSSFKYIFKKNLFKLVLPSYLKTTNQDKLKIISSFDNFSTYFRGFFENRKNIFTKKPISTSIAYRIVHDNFPKFLDNIRCFNVWQTECPQLIVKADNYLKSKNVIAKDKSLANYFTVGAYDYFLSQNGIDFYNNIIGGLPAFAGHEKIQGLNEFINQECQKDSELKSKLKNRHAFKMAVLFKQILSDREKSFVIDEFESDAQVIDAVKNFYAEQCKDNNVIFNLLNLIKNIAFLSDDELDGIFIEGKYLSSVSQKLYSDWSKLRNDIEDSANSKQGNKELAKKIKTNKGDVEKAISKYEFSLSELNSIVHDNTKFSDLLSCTLHKVASEKLVKVNEGDWPKHLKNNEEKQKIKEPLDALLEIYNTLLIFNCKSFNKNGNFYVDYDRCINELSSVVYLYNKTRNYCTKKPYNTDKFKLNFNSPQLGEGFSKSKENDCLTLLFKKDDNYYVGIIRKGAKINFDDTQAIADNTDNCIFKMNYFLLKDAKKFIPKCSIQLKEVKAHFKKSEDDYILSDKEKFASPLVIKKSTFLLATAHVKGKKGNIKKFQKEYSKENPTEYRNSLNEWIAFCKEFLKTYKAATIFDITTLKKAEEYADIVEFYKDVDNLCYKLEFCPIKTSFIENLIDNGDLYLFRINNKDFSSKSTGTKNLHTLYLQAIFDERNLNNPTIMLNGGAELFYRKESIEQKNRITHKAGSILVNKVCKDGTSLDDKIRNEIYQYENKFIDTLSDEAKKVLPNVIKKEATHDITKDKRFTSDKFFFHCPLTINYKEGDTKQFNNEVLSFLRGNPDINIIGIDRGERNLIYVTVINQKGEILDSVSFNTVTNKSSKIEQTVDYEEKLAVREKERIEAKRSWDSISKIATLKEGYLSAIVHEICLLMIKHNAIVVLENLNAGFKRIRGGLSEKSVYQKFEKMLINKLNYFVSKKESDWNKPSGLLNGLQLSDQFESFEKLGIQSGFIFYVPAAYTSKIDPTTGFANVLNLSKVRNVDAIKSFFSNFNEISYSKKEALFKFSFDLDSLSKKGFSSFVKFSKSKWNVYTFGERIIKPKNKQGYREDKRINLTFEMKKLLNEYKVSFDLENNLIPNLTSANLKDTFWKELFFIFKTTLQLRNSVTNGKEDVLISPVKNAKGEFFVSGTHNKTLPQDCDANGAYHIALKGLMILERNNLVREEKDTKKIMAISNVDWFEYVQKRRGVL

SEQ ID NO: 39 - protein, 10 aa, synthetic construct
  source 1..10: note = peptide motif
YLFQIYNKDF

SEQ ID NO: 40 - protein, 7 aa, synthetic construct
  source 1..7: note = NLS of SV40 large T-antigen
PKKKRKV

SEQ ID NO: 41 - protein, 16 aa, synthetic construct
  source 1..16: note = nucleoplasmin bipartite NLS
KRPAATKKAGQAKKKK

SEQ ID NO: 42 - protein, 9 aa, synthetic construct
  source 1..9: note = c-myc NLS
PAAKRVKLD

SEQ ID NO: 43 - protein, 11 aa, synthetic construct
  source 1..11: note = c-myc NLS
RQRRNELKRSP

SEQ ID NO: 44 - protein, 38 aa, synthetic construct
  source 1..38: note = hRNPA1 M9 NLS
NQSSNFGPMKGGNFGGRSSGPYGGGGQYFAKPRNQGGY

SEQ ID NO: 45 - protein, 42 aa, synthetic construct
  source 1..42: note = importin-a IBB domain NLS
RMRIZFKNKGKDTAELRRRRVEVSVELRKAKKDEQILKRRNV

SEQ ID NO: 46 - protein, 8 aa, synthetic construct
  source 1..8: note = myoma T protein NLS
VSRKRPRP

SEQ ID NO: 47 - protein, 8 aa, synthetic construct
  source 1..8: note = myoma T protein NLS
PPKKARED

SEQ ID NO: 48 - protein, 8 aa, synthetic construct
  source 1..8: note = human p53 NLS
PQPKKKPL

SEQ ID NO: 49 - protein, 12 aa, synthetic construct
  source 1..12: note = mouse c-abl IV NLS
SALIKKKKKMAP

SEQ ID NO: 50 - protein, 5 aa, synthetic construct
  source 1..5: note = influenza virus NS1 NLS
DRLRR

SEQ ID NO: 51 - protein, 7 aa, synthetic construct
  source 1..7: note = influenza virus NS1 NLS
PKQKKRK

SEQ ID NO: 52 - protein, 10 aa, synthetic construct
  source 1..10: note = hepatitis virus d antigen NLS
RKLKKKIKKL

SEQ ID NO: 53 - protein, 10 aa, synthetic construct
  source 1..10: note = mouse Mx1 protein NLS
REKKKFLKRR

SEQ ID NO: 54 - protein, 20 aa, synthetic construct
  source 1..20: note = human poly(ADP-ribose) polymerase NLS
KRKGDEVDGVDEVAKKKSKK

SEQ ID NO: 55 - protein, 17 aa, synthetic construct
  source 1..17: note = human glucocorticoid receptor NLS
RKCLQAGMNLEARKTKK

SEQ ID NO: 56 - protein, 9 aa, synthetic construct
  source 1..9: note = synthetic NLS motif
PAAKKKKLD

SEQ ID NO: 57 - RNA, 19 nt, synthetic construct
  source 1..19: mol_type = other RNA; note = scaffold sequence
taatttctactcttgtaga

SEQ ID NO: 58 - RNA, 15 nt, synthetic construct
  source 1..15: mol_type = other RNA; note = scaffold sequence
atctacaacagtaga

SEQ ID NO: 59 - RNA, 15 nt, synthetic construct
  source 1..15: mol_type = other RNA; note = scaffold sequence
atctacaaaagtaga

SEQ ID NO: 60 - RNA, 20 nt, synthetic construct
  source 1..20: mol_type = other RNA; note = scaffold sequence
ggaatttctactcttgtaga

SEQ ID NO: 61 - RNA, 19 nt, synthetic construct
  source 1..19: mol_type = other RNA; note = scaffold sequence
taattcccactcttgtggg

SEQ ID NO: 62 - RNA, 15 nt, synthetic construct
  source 1..15: mol_type = other RNA; note = scaffold sequence
atctacaagagtaga

SEQ ID NO: 63 - RNA, 15 nt, synthetic construct
  source 1..15: mol_type = other RNA; note = scaffold sequence
atctacactagtaga

SEQ ID NO: 64 - RNA, 20 nt, synthetic construct
  source 1..20: mol_type = other RNA; note = scaffold sequence
taatttctactaagtgtaga

SEQ ID NO: 65 - RNA, 21 nt, synthetic construct
  source 1..21: mol_type = other RNA; note = scaffold sequence
taattttctacttgttgtaga

SEQ ID NO: 66 - RNA, 18 nt, synthetic construct
  source 1..18: mol_type = other RNA; note = scaffold sequence
aatttctactgttgtaga

SEQ ID NO: 67 - RNA, 19 nt, synthetic construct
  source 1..19: mol_type = other RNA; note = scaffold sequence
gaatttctactgttgtaga

SEQ ID NO: 68 - RNA, 36 nt, synthetic construct
  source 1..36: mol_type = other RNA; note = scaffold sequence
gtctaaaggtaccaccaaatttctactgttgtagat

SEQ ID NO: 69 - RNA, 36 nt, synthetic construct
  source 1..36: mol_type = other RNA; note = scaffold sequence
gcttagaacctttaaataatttctactattgtagat

SEQ ID NO: 70 - RNA, 10 nt, synthetic construct
  source 1..10: mol_type = other RNA; note = modulator sequence
taatttctac

SEQ ID NO: 71 - skipped

SEQ ID NO: 72 - RNA, 11 nt, synthetic construct
  source 1..11: mol_type = other RNA; note = modulator sequence
ggaatttctac

SEQ ID NO: 73 - RNA, 10 nt, synthetic construct
  source 1..10: mol_type = other RNA; note = modulator sequence
taattcccac

SEQ ID NO: 74 - RNA, 12 nt, synthetic construct
  source 1..12: mol_type = other RNA; note = modulator sequence
taattttctact

SEQ ID NO: 75 - skipped

SEQ ID NO: 76 - RNA, 10 nt, synthetic construct
  source 1..10: mol_type = other RNA; note = modulator sequence
gaatttctac

SEQ ID NO: 77 - RNA, 10 nt, synthetic construct
  source 1..10: mol_type = other RNA; note = modulator sequence
aaatttctac

SEQ ID NO: 78 - DNA, 508 bp, synthetic construct
  source 1..508: mol_type = other DNA; note = CMV
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 79 - DNA, 81 bp, synthetic construct
  source 1..81: mol_type = other DNA; note = SCP
gtacttatataagggggtgggggcgcgttcgtcctcagtcgcgatcgaacactcgagccgagcagacgtgcctacggaccg

SEQ ID NO: 80 - DNA, 385 bp, synthetic construct
  source 1..385: mol_type = other DNA; note = CMVe-SCP
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtacttatataagggggtgggggcgcgttcgtcctcagtcgcgatcgaacactcgagccgagcagacgtgcctacggaccg

SEQ ID NO: 81 - DNA, 947 bp, synthetic construct
  source 1..947: mol_type = other DNA; note = CMVmax
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggg

SEQ ID NO: 82 - DNA, 195 bp, synthetic construct
  source 1..195: mol_type = other DNA; note = JET
gaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgaca

SEQ ID NO: 83 - DNA, 1723 bp, synthetic construct
  source 1..1723: mol_type = other DNA; note = CAG
atctcgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

SEQ ID NO: 84 - DNA, 511 bp, synthetic construct
  source 1..511: mol_type = other DNA; note = PGK
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 85 - DNA, 1195 bp, synthetic construct
  source 1..1195: mol_type = other DNA; note = EF-1a
gaattcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacatcatttt

SEQ ID NO: 86 - RNA, 21 nt, synthetic construct
  source 1..21: mol_type = other RNA; note = crCD247_1
accgcggccatcctgcaggca

SEQ ID NO: 87 - RNA, 21 nt, synthetic construct
  source 1..21: mol_type = other RNA; note = crCD247_2
tgagggaaaggacaagatgaa

SEQ ID NO: 88 - RNA, 21 nt, synthetic construct
  source 1..21: mol_type = other RNA; note = crCD247_3
ggatccagcaggccaaagctc

SEQ ID NO: 89 - RNA, 21 nt, synthetic construct
  source 1..21: mol_type = other RNA; note = crCD247_4
ctagcagagaaggaagaaccc

SEQ ID NO: 90 - RNA, 21 nt, synthetic construct
  source 1..21: mol_type = other RNA; note = crCD247_5
tgtgttgcagttcagcaggag

SEQ ID NO: 91 - RNA, 21 nt, synthetic construct
  source 1..21: mol_type = other RNA; note = crCD247_6
ctgagggttcttccttctctg

SEQ ID NO: 92 - RNA, 21 nt, synthetic construct
  source 1..21: mol_type = other RNA; note = crCD247_7
ccgttgtctttcctagcagag

SEQ ID NO: 93 - RNA, 21 nt, synthetic construct
  source 1..21: mol_type = other RNA; note = crCD247_8
tgcagttcctgcagaagaggg

SEQ ID NO: 94 - RNA, 21 nt, synthetic construct
  source 1..21: mol_type = other RNA; note = crCD247_9
tgcaggaactgcagaaagata

SEQ ID NO: 95 - RNA, 21 nt, synthetic construct
  source 1..21: mol_type = other RNA; note = crCD247_10
atcccaatctcactgtaggcc

SEQ ID NO: 96 - RNA, 21 nt, synthetic construct
  source 1..21: mol_type = other RNA; note = crCD247_11
catcccaatctcactgtaggc

SEQ ID NO: 97 - RNA, 21 nt, synthetic construct
  source 1..21: mol_type = other RNA; note = crCD247_12
ctcatttcactcccaaacaac

SEQ ID NO: 98 - RNA, 21 nt, synthetic construct
  source 1..21: mol_type = other RNA; note = crCD247_13
tcatttcactcccaaacaacc

SEQ ID NO: 99 - RNA, 21 nt, synthetic construct
  source 1..21: mol_type = other RNA; note = crCD247_14
actcccaaacaaccagcgccg

SEQ ID NO: 100 - RNA, 21 nt, synthetic construct
  source 1..21: mol_type = other RNA; note = crCD247_15
cgttatagagctggttctggc

SEQ ID NO: 101 - RNA, 21 nt, synthetic construct
  source 1..21: mol_type = other RNA; note = crCD247_16
ttttctgatttgctttcacgc

SEQ ID NO: 102 - RNA, 21 nt, synthetic construct
  source 1..21: mol_type = other RNA; note = crCD247_17
tgatttgctttcacgccaggg

SEQ ID NO: 103 - RNA, 21 nt, synthetic construct
  source 1..21: mol_type = other RNA; note = crCD247_18
ctttcacgccagggtctcagt

SEQ ID NO: 104 - RNA, 21 nt, synthetic construct
  source 1..21: mol_type = other RNA; note = crCD247_19
acgccagggtctcagtacagc

SEQ ID NO: 105 - RNA, 21 nt, synthetic construct
  source 1..21: mol_type = other RNA; note = crCD247_20
cggagggtctacggcgaggct

SEQ ID NO: 106 - RNA, 21 nt, synthetic construct
  source 1..21: mol_type = other RNA; note = crCD247_21
ttatctgttataggagctcaa

SEQ ID NO: 107 - RNA, 21 nt, synthetic construct
  source 1..21: mol_type = other RNA; note = crCD247_22
tctgttataggagctcaatct

SEQ ID NO: 108 - RNA, 21 nt, synthetic construct
  source 1..21: mol_type = other RNA; note = crCD247_23
tccaaaacatcgtactcctct

SEQ ID NO: 109 - RNA, 21 nt, synthetic construct
  source 1..21: mol_type = other RNA; note = crCD247_24
cccccatctcagggtcccggc

SEQ ID NO: 110 - RNA, 21 nt, synthetic construct
  source 1..21: mol_type = other RNA; note = crCD247_25
gacaagagacgtggccgggac

SEQ ID NO: 111 - RNA, 21 nt, synthetic construct
  source 1..21: mol_type = other RNA; note = crCD247_26
tctccctctaacgtcttcccg

SEQ ID NO: 112 - RNA, 21 nt, synthetic construct
  source 1..21: mol_type = other RNA; note = crCTLA4_1
cctggagatgcatactcacac

SEQ ID NO: 113 - RNA, 21 nt, synthetic construct
  source 1..21: mol_type = other RNA; note = crCTLA4_2
cagaagacagggatgaagaga

SEQ ID NO: 114 - RNA, 21 nt, synthetic construct
  source 1..21: mol_type = other RNA; note = crCTLA4_3
cactggaggtgcccgtgcaga

SEQ ID NO: 115 - RNA, 21 nt, synthetic construct
  source 1..21: mol_type = other RNA; note = crCTLA4_4
tgtgtgagtatgcatctccag

SEQ ID NO: 116 - RNA, 21 nt, synthetic construct
  source 1..21: mol_type = other RNA; note = crCTLA4_5
agcggcacaaggctcagctga

SEQ ID NO: 117 - RNA, 21 nt, synthetic construct
  source 1..21: mol_type = other RNA; note = crCTLA4_6
tgccgctgaaatccaaggcaa

SEQ ID NO: 118 - RNA, 21 nt, synthetic construct
  source 1..21: mol_type = other RNA; note = crCTLA4_7
tccatgctagcaatgcacgtg

SEQ ID NO: 119 - RNA, 21 nt, synthetic construct
  source 1..21: mol_type = other RNA; note = crCTLA4_8
ccatgctagcaatgcacgtgg

SEQ ID NO: 120 - RNA, 21 nt, synthetic construct
  source 1..21: mol_type = other RNA; note = crCTLA4_9
gtgtgtgagtatgcatctcca

SEQ ID NO: 121 - RNA, 21 nt, synthetic construct
  source 1..21: mol_type = other RNA; note = crCTLA4_10
gcctggagatgcatactcaca

SEQ ID NO: 122 - RNA, 21 nt, synthetic construct
  source 1..21: mol_type = other RNA; note = crCTLA4_11
ggcaggctgacagccaggtga

SEQ ID NO: 123 - RNA, 21 nt, synthetic construct
  source 1..21: mol_type = other RNA; note = crCTLA4_12
agtcacctggctgtcagcctg

SEQ ID NO: 124 - RNA, 21 nt, synthetic construct
  source 1..21: mol_type = other RNA; note = crCTLA4_13
ctagatgattccatctgcacg

SEQ ID NO: 125 - RNA, 21 nt, synthetic construct
  source 1..21: mol_type = other RNA; note = crCTLA4_14
ccttggatttcagcggcacaa

SEQ ID NO: 126 - RNA, 21 nt, synthetic construct
  source 1..21: mol_type = other RNA; note = crCTLA4_15
atttccactggaggtgcccgt

SEQ ID NO: 127 - RNA, 21 nt, synthetic construct
  source 1..21: mol_type = other RNA; note = crCTLA4_16
gatagtgaggttcacttgatt

SEQ ID NO: 128 - RNA, 21 nt, synthetic construct
  source 1..21: mol_type = other RNA; note = crCTLA4_17
cagatgtagagtcccgtgtcc

SEQ ID NO: 129 - RNA, 21 nt, synthetic construct
  source 1..21: mol_type = other RNA; note = crCTLA4_18
ctcaccaattacataaatctg

SEQ ID NO: 130 - RNA, 21 nt, synthetic construct
  source 1..21: mol_type = other RNA; note = crCTLA4_19
gctcaccaattacataaatct

SEQ ID NO: 131 - RNA, 21 nt, synthetic construct
  source 1..21: mol_type = other RNA; note = crCTLA4_20
gttttctgttgcagatccaga

SEQ ID NO: 132 - RNA, 21 nt, synthetic construct
  source 1..21: mol_type = other RNA; note = crCTLA4_21
ttttctgttgcagatccagaa

SEQ ID NO: 133 - RNA, 21 nt, synthetic construct
  source 1..21: mol_type = other RNA; note = crCTLA4_22
ctgttgcagatccagaaccgt

SEQ ID NO: 134 - RNA, 21 nt, synthetic construct
  source 1..21: mol_type = other RNA; note = crCTLA4_23
ctcctctggatccttgcagca

SEQ ID NO: 135 - RNA, 21 nt, synthetic construct
  source 1..21: mol_type = other RNA; note = crCTLA4_24
cagcagttagttcggggttgt

SEQ ID NO: 136 - RNA, 21 nt, synthetic construct
  source 1..21: mol_type = other RNA; note = crCTLA4_25
gatttcagcggcacaaggctc

SEQ ID NO: 137 - RNA, 21 nt, synthetic construct
  source 1..21: mol_type = other RNA; note = crCTLA4_26
tttatagctttctcctcacag

SEQ ID NO: 138 - RNA, 21 nt, synthetic construct
  source 1..21: mol_type = other RNA; note = crCTLA4_27
ctcctcacagctgtttctttg

SEQ ID NO: 139 - RNA, 21 nt, synthetic construct
  source 1..21: mol_type = other RNA; note = crCTLA4_28
tcctcacagctgtttctttga

SEQ ID NO: 140 - RNA, 21 nt, synthetic construct
  source 1..21: mol_type = other RNA; note = crCTLA4_29
gctcaaagaaacagctgtgag

SEQ ID NO: 141 - RNA, 21 nt, synthetic construct
  source 1..21: mol_type = other RNA; note = crCTLA4_30
tttttgtgtttgacagctaaa

SEQ ID NO: 142 - RNA, 21 nt, synthetic construct
  source 1..21: mol_type = other RNA; note = crCTLA4_31
tgtgtttgacagctaaagaaa

SEQ ID NO: 143 - RNA, 21 nt, synthetic construct
  source 1..21: mol_type = other RNA; note = crCTLA4_32
acagctaaagaaaagaagccc

SEQ ID NO: 144 - RNA, 21 nt, synthetic construct
  source 1..21: mol_type = other RNA; note = crCTLA4_33
cacatagacccctgttgtaag

SEQ ID NO: 145 - RNA, 21 nt, synthetic construct
  source 1..21: mol_type = other RNA; note = crCTLA4_34
cacattctggctctgttgggg

SEQ ID NO: 146 - RNA, 21 nt, synthetic construct
  source 1..21: mol_type = other RNA; note = crCTLA4_35
tcacattctggctctgttggg

SEQ ID NO: 147 - RNA, 21 nt, synthetic construct
  source 1..21: mol_type = other RNA; note = crCTLA4_36
agccttattttattcccatca

SEQ ID NO: 148 - RNA, 21 nt, synthetic construct
  source 1..21: mol_type = other RNA; note = crCTLA4_37
tcaattgatgggaataaaata

SEQ ID NO: 149 - RNA, 21 nt, synthetic construct
  source 1..21: mol_type = other RNA; note = crCTLA4_38
ttcttctcttcatccctgtct

SEQ ID NO: 150 - RNA, 21 nt, synthetic construct
  source 1..21: mol_type = other RNA; note = crCTLA4_39
gcagaagacagggatgaagag

SEQ ID NO: 151 - RNA, 21 nt, synthetic construct
  source 1..21: mol_type = other RNA; note = crCTLA4_40
ggcttttccatgctagcaatg

SEQ ID NO: 152 - RNA, 21 nt, synthetic construct
  source 1..21: mol_type = other RNA; note = crCTLA4_41
gcttttccatgctagcaatgc

SEQ ID NO: 153 - RNA, 21 nt, synthetic construct
  source 1..21: mol_type = other RNA; note = crLAG3_1
gggtgcatacctgtctggctg

SEQ ID NO: 154 - RNA, 21 nt, synthetic construct
  source 1..21: mol_type = other RNA; note = crLAG3_2
ggtcacctggatccctgggga

SEQ ID NO: 155 - RNA, 21 nt, synthetic construct
  source 1..21: mol_type = other RNA; note = crLAG3_3
tcaggaccttggctggaggca

SEQ ID NO: 156 - RNA, 21 nt, synthetic construct
  source 1..21: mol_type = other RNA; note = crLAG3_4
ccagccttggcaatgccagct

SEQ ID NO: 157 - RNA, 21 nt, synthetic construct
  source 1..21: mol_type = other RNA; note = crLAG3_5
tgaggtgactccagtatctgg

SEQ ID NO: 158 - RNA, 21 nt, synthetic construct
  source 1..21: mol_type = other RNA; note = crLAG3_6
ctgtttctgcagccgctttgg

SEQ ID NO: 159 - RNA, 21 nt, synthetic construct
  source 1..21: mol_type = other RNA; note = crLAG3_7
cacagtgactgccagcccccc

SEQ ID NO: 160 - RNA, 21 nt, synthetic construct
  source 1..21: mol_type = other RNA; note = crLAG3_8
gaactgctccttcagccgccc

SEQ ID NO: 161 - RNA, 21 nt, synthetic construct
  source 1..21: mol_type = other RNA; note = crLAG3_9
agccgccctgaccgcccagcc

SEQ ID NO: 162 - RNA, 21 nt, synthetic construct
  source 1..21: mol_type = other RNA; note = crLAG3_10
cgctaagtggtgatgggggga

SEQ ID NO: 163 - RNA, 21 nt, synthetic construct
  source 1..21: mol_type = other RNA; note = crLAG3_11
ccgctaagtggtgatgggggg

SEQ ID NO: 164 - RNA, 21 nt, synthetic construct
  source 1..21: mol_type = other RNA; note = crLAG3_12
gcggaaagcttcctcttcctg

SEQ ID NO: 165 - RNA, 21 nt, synthetic construct
  source 1..21: mol_type = other RNA; note = crLAG3_13
gggcaggaagaggaagctttc

SEQ ID NO: 166 - RNA, 21 nt, synthetic construct
  source 1..21: mol_type = other RNA; note = crLAG3_14
ctcttcctgccccaagtcagc

SEQ ID NO: 167 - RNA, 21 nt, synthetic construct
  source 1..21: mol_type = other RNA; note = crLAG3_15
aacgtctccatcatgtataac

SEQ ID NO: 168 - RNA, 21 nt, synthetic construct
  source 1..21: mol_type = other RNA; note = crLAG3_16
cttttctcttcaggtctggag

SEQ ID NO: 169 - RNA, 21 nt, synthetic construct
  source 1..21: mol_type = other RNA; note = crLAG3_17
tgcagccgctttgggtggctc

SEQ ID NO: 170 - RNA, 21 nt, synthetic construct
  source 1..21: mol_type = other RNA; note = crLAG3_18
ctcttcaggtctggagccccc

SEQ ID NO: 171 - RNA, 21 nt, synthetic construct
  source 1..21: mol_type = other RNA; note = crLAG3_19
acagtgtacgctggagcaggt

SEQ ID NO: 172 - RNA, 21 nt, synthetic construct
  source 1..21: mol_type = other RNA; note = crLAG3_20
gcagtgaggaaagaccgggtc

SEQ ID NO: 173 - RNA, 21 nt, synthetic construct
  source 1..21: mol_type = other RNA; note = crLAG3_21
ctcactgccaagtggactcct

SEQ ID NO: 174 - RNA, 21 nt, synthetic construct
  source 1..21: mol_type = other RNA; note = crLAG3_22
acccttcgactagaggatgtg

SEQ ID NO: 175 - RNA, 21 nt, synthetic construct
  source 1..21: mol_type = other RNA; note = crLAG3_23
cccttcgactagaggatgtga

SEQ ID NO: 176 - RNA, 21 nt, synthetic construct
  source 1..21: mol_type = other RNA; note = crLAG3_24
gactagaggatgtgagccagg

SEQ ID NO: 177 - RNA, 21 nt, synthetic construct
  source 1..21: mol_type = other RNA; note = crLAG3_25
ccacctgaggctgacctgtga

SEQ ID NO: 178 - RNA, 21 nt, synthetic construct
  source 1..21: mol_type = other RNA; note = crLAG3_26
cccacctgaggctgacctgtg

SEQ ID NO: 179 - RNA, 21 nt, synthetic construct
  source 1..21: mol_type = other RNA; note = crLAG3_27
tactcttttcagtgactccca

SEQ ID NO: 180 - RNA, 21 nt, synthetic construct
  source 1..21: mol_type = other RNA; note = crLAG3_28
acctggagccacccaaagcgg

SEQ ID NO: 181 - RNA, 21 nt, synthetic construct
  source 1..21: mol_type = other RNA; note = crLAG3_29
cagtgactcccaaatcctttg

SEQ ID NO: 182 - RNA, 21 nt, synthetic construct
  source 1..21: mol_type = other RNA; note = crLAG3_30
cccagggatccaggtgaccca

SEQ ID NO: 183 - RNA, 21 nt, synthetic construct
  source 1..21: mol_type = other RNA; note = crLAG3_31
gggtcacctggatccctgggg

SEQ ID NO: 184 - RNA, 21 nt, synthetic construct
  source 1..21: mol_type = other RNA; note = crLAG3_32
gtgaggtgactccagtatctg

SEQ ID NO: 185 - RNA, 21 nt, synthetic construct
  source 1..21: mol_type = other RNA; note = crLAG3_33
gtgtggagctctctggacacc

SEQ ID NO: 186 - RNA, 21 nt, synthetic construct
  source 1..21: mol_type = other RNA; note = crLAG3_34
tgtggagctctctggacaccc

SEQ ID NO: 187 - RNA, 21 nt, synthetic construct
  source 1..21: mol_type = other RNA; note = crLAG3_35
gctggaggcacaggaggccca

SEQ ID NO: 188 - RNA, 21 nt, synthetic construct
  source 1..21: mol_type = other RNA; note = crLAG3_36
gctcacctagtgaagcctctc

SEQ ID NO: 189 - RNA, 21 nt, synthetic construct
  source 1..21: mol_type = other RNA; note = crLAG3_37
cccagccttggcaatgccagc

SEQ ID NO: 190 - RNA, 21 nt, synthetic construct
  source 1..21: mol_type = other RNA; note = crLAG3_38
gcaatgccagctgtaccaggg

SEQ ID NO: 191 - RNA, 21 nt, synthetic construct
  source 1..21: mol_type = other RNA; note = crLAG3_39
ttggagcagcagtgtacttca

SEQ ID NO: 192 - RNA, 21 nt, synthetic construct
  source 1..21: mol_type = other RNA; note = crLAG3_40
acagagctgtctagcccaggt

SEQ ID NO: 193 - RNA, 21 nt, synthetic construct
  source 1..21: mol_type = other RNA; note = crLAG3_41
ctccataggtgcccaacgctc

SEQ ID NO: 194 - RNA, 21 nt, synthetic construct
  source 1..21: mol_type = other RNA; note = crLAG3_42
tccataggtgcccaacgctct

SEQ ID NO: 195 - RNA, 21 nt, synthetic construct
  source 1..21: mol_type = other RNA; note = crLAG3_43
tcatccttggtgtcctttctc

SEQ ID NO: 196 - RNA, 21 nt, synthetic construct
  source 1..21: mol_type = other RNA; note = crLAG3_44
gtgtcctttctctgctccttt

SEQ ID NO: 197 - RNA, 21 nt, synthetic construct
  source 1..21: mol_type = other RNA; note = crLAG3_45
ctctgctccttttggtgactg

SEQ ID NO: 198 - RNA, 21 nt, synthetic construct
  source 1..21: mol_type = other RNA; note = crLAG3_46
tgcgaagagcaggggtcactt

SEQ ID NO: 199 - RNA, 21 nt, synthetic construct
  source 1..21: mol_type = other RNA; note = crLAG3_47
tggtgactggagcctttggct

SEQ ID NO: 200 - RNA, 21 nt, synthetic construct
  source 1..21: mol_type = other RNA; note = crLAG3_48
ggtgactggagcctttggctt

SEQ ID NO: 201 - RNA, 21 nt, synthetic construct
  source 1..21: mol_type = other RNA; note = crLAG3_49
ggctttcacctttggagaaga

SEQ ID NO: 202 - RNA, 21 nt, synthetic construct
  source 1..21: mol_type = other RNA; note = crLAG3_50
gctttcacctttggagaagac

SEQ ID NO: 203 - RNA, 21 nt, synthetic construct
  source 1..21: mol_type = other RNA; note = crLAG3_51
ctctaaggcagaaaatcgtct

SEQ ID NO: 204 - RNA, 21 nt, synthetic construct
  source 1..21: mol_type = other RNA; note = crLAG3_52
ctgccttagagcaagggattc

SEQ ID NO: 205 - RNA, 21 nt, synthetic construct
  source 1..21: mol_type = other RNA; note = crLAG3_53
gagcaagggattcaccctccg

SEQ ID NO: 206 - RNA, 21 nt, synthetic construct
  source 1..21: mol_type = other RNA; note = crLAG3_54
ccgcccagtggcccgcccgct

SEQ ID NO: 207 - RNA, 21 nt, synthetic construct
  source 1..21: mol_type = other RNA; note = crLAG3_55
tcgctatggctgcgcccagcc

SEQ ID NO: 208 - RNA, 21 nt, synthetic construct
  source 1..21: mol_type = other RNA; note = crLAG3_56
tccttgcacagtgactgccag

SEQ ID NO: 209 - RNA, 21 nt, synthetic construct
  source 1..21: mol_type = other RNA; note = crPDCD1_1
gcacgaagctctccgatgtgt

SEQ ID NO: 210 - RNA, 21 nt, synthetic construct
  source 1..21: mol_type = other RNA; note = crPDCD1_2
tctgcagggacaataggagcc

SEQ ID NO: 211 - RNA, 21 nt, synthetic construct
  source 1..21: mol_type = other RNA; note = crPDCD1_3
cagtggcgagagaagaccccg

SEQ ID NO: 212 - RNA, 21 nt, synthetic construct
  source 1..21: mol_type = other RNA; note = crPDCD1_4
ctagcggaatgggcacctcat

SEQ ID NO: 213 - RNA, 21 nt, synthetic construct
  source 1..21: mol_type = other RNA; note = crPDCD1_5
gtgctaaactggtaccgcatg

SEQ ID NO: 214 - RNA, 21 nt, synthetic construct
  source 1..21: mol_type = other RNA; note = crPDCD1_6
aacctgacctgggacagtttc

SEQ ID NO: 215 - RNA, 21 nt, synthetic construct
  source 1..21: mol_type = other RNA; note = crPDCD1_7
tccgtctggttgctggggctc

SEQ ID NO: 216 - RNA, 21 nt, synthetic construct
  source 1..21: mol_type = other RNA; note = crPDCD1_8
cccgaggaccgcagccagccc

SEQ ID NO: 217 - RNA, 21 nt, synthetic construct
  source 1..21: mol_type = other RNA; note = crPDCD1_9
cgtgtcacacaactgcccaac

SEQ ID NO: 218 - RNA, 21 nt, synthetic construct
  source 1..21: mol_type = other RNA; note = crPDCD1_10
cacatgagcgtggtcagggcc

SEQ ID NO: 219 - RNA, 21 nt, synthetic construct
  source 1..21: mol_type = other RNA; note = crPDCD1_11
gatctgcgccttgggggccag

SEQ ID NO: 220 - RNA, 21 nt, synthetic construct
  source 1..21: mol_type = other RNA; note = crPDCD1_12
atctgcgccttgggggccagg

SEQ ID NO: 221 - RNA, 21 nt, synthetic construct
  source 1..21: mol_type = other RNA; note = crPDCD1_13
ggggccagggagatggcccca

SEQ ID NO: 222 - RNA, 21 nt, synthetic construct
  source 1..21: mol_type = other RNA; note = crPDCD1_14
gtgcccttccagagagaaggg

SEQ ID NO: 223 - RNA, 21 nt, synthetic construct
  source 1..21: mol_type = other RNA; note = crPDCD1_15
tgcccttccagagagaagggc

SEQ ID NO: 224 - RNA, 21 nt, synthetic construct
  source 1..21: mol_type = other RNA; note = crPDCD1_16
ccttccgctcacctccgcctg

SEQ ID NO: 225 - RNA, 21 nt, synthetic construct
  source 1..21: mol_type = other RNA; note = crPDCD1_17
cagagagaagggcagaagtgc

SEQ ID NO: 226 - RNA, 21 nt, synthetic construct
  source 1..21: mol_type = other RNA; note = crPDCD1_18
tgcccttctctctggaagggc

SEQ ID NO: 227 - RNA, 21 nt, synthetic construct
  source 1..21: mol_type = other RNA; note = crPDCD1_19
gaactggccggctggcctggg

SEQ ID NO: 228 - RNA, 21 nt, synthetic construct
  source 1..21: mol_type = other RNA; note = crPDCD1_20
ctcctcaaagaaggaggaccc

SEQ ID NO: 229 - RNA, 21 nt, synthetic construct
  source 1..21: mol_type = other RNA; note = crPDCD1_21
tcctcaaagaaggaggacccc

SEQ ID NO: 230 - RNA, 21 nt, synthetic construct
  source 1..21: mol_type = other RNA; note = crPDCD1_22
tctcgccactggaaatccagc

SEQ ID NO: 231 - RNA, 21 nt, synthetic construct
  source 1..21: mol_type = other RNA; note = crPDCD1_23
cctagcggaatgggcacctca

SEQ ID NO: 232 - RNA, 21 nt, synthetic construct
  source 1..21: mol_type = other RNA; note = crPDCD1_24
cgctcacctccgcctgagcag

SEQ ID NO: 233 - RNA, 21 nt, synthetic construct
  source 1..21: mol_type = other RNA; note = crPDCD1_25
gcccctctgaccggcttcctt

SEQ ID NO: 234 - RNA, 21 nt, synthetic construct
  source 1..21: mol_type = other RNA; note = crPDCD1_26
tccactgctcaggcggaggtg

SEQ ID NO: 235 - RNA, 21 nt, synthetic construct
  source 1..21: mol_type = other RNA; note = crPDCD1_27
tccccagccctgctcgtggtg

SEQ ID NO: 236 - RNA, 21 nt, synthetic construct
  source 1..21: mol_type = other RNA; note = crPDCD1_28
ggtcaccacgagcagggctgg

SEQ ID NO: 237 - RNA, 21 nt, synthetic construct
  source 1..21: mol_type = other RNA; note = crPDCD1_29
acctgcagcttctccaacaca

SEQ ID NO: 238 - RNA, 21 nt, synthetic construct
  source 1..21: mol_type = other RNA; note = crPDCD1_30
tccaacacatcggagagcttc

SEQ ID NO: 239 - RNA, 21 nt, synthetic construct
  source 1..21: mol_type = other RNA; note = crPTPN1_1
cctgacagcgaatcataacat

SEQ ID NO: 240 - RNA, 21 nt, synthetic construct
  source 1..21: mol_type = other RNA; note = crPTPN1_2
attccaacttacctaacggaa

SEQ ID NO: 241 - RNA, 21 nt, synthetic construct
  source 1..21: mol_type = other RNA; note = crPTPN1_3
tgtgcgcactggtgatgacaa

SEQ ID NO: 242 - RNA, 21 nt, synthetic construct
  source 1..21: mol_type = other RNA; note = crPTPN11_4
caatctgctcacctgcttgag

SEQ ID NO: 243 - RNA, 21 nt, synthetic construct
  source 1..21: mol_type = other RNA; note = crPTPN11_5
ttctagttgatcataccaggg

SEQ ID NO: 244 - RNA, 21 nt, synthetic construct
  source 1..21: mol_type = other RNA; note = crPTPN11_6
ataacttacctcaaattcttc

SEQ ID NO: 245 - RNA, 21 nt, synthetic construct
  source 1..21: mol_type = other RNA; note = crPTPN11_7
cctaacggaaagtgtgaagtc

SEQ ID NO: 246 - RNA, 21 nt, synthetic construct
  source 1..21: mol_type = other RNA; note = crPTPN11_8
cagacactacaacaacaggag

SEQ ID NO: 247 - RNA, 21 nt, synthetic construct
  source 1..21: mol_type = other RNA; note = crPTPN11_9
ggtggtttcatggacatctct

SEQ ID NO: 248 - RNA, 21 nt, synthetic construct
  source 1..21: mol_type = other RNA; note = crPTPN11_10
ccagagagatgtccatgaaac

SEQ ID NO: 249 - RNA, 21 nt, synthetic construct
  source 1..21: mol_type = other RNA; note = crPTPN6_1
tatgacctgtatggaggggag

SEQ ID NO: 250 - RNA, 21 nt, synthetic construct
  source 1..21: mol_type = other RNA; note = crPTPN6_2
cgactctgacagagctggtgg

SEQ ID NO: 251 - RNA, 21 nt, synthetic construct
  source 1..21: mol_type = other RNA; note = crPTPN6_3
cagaagcaggaggtgaagaac

SEQ ID NO: 252 - RNA, 21 nt, synthetic construct
  source 1..21: mol_type = other RNA; note = crPTPN6_4
actgccccccacccaggcctg

SEQ ID NO: 253 - RNA, 21 nt, synthetic construct
  source 1..21: mol_type = other RNA; note = crPTPN6_5
tgggccctactctgtgaccaa

SEQ ID NO: 254 - RNA, 21 nt, synthetic construct
  source 1..21: mol_type = other RNA; note = crPTPN6_6
accgagacctcagtgggctgg

SEQ ID NO: 255 - RNA, 21 nt, synthetic construct
  source 1..21: mol_type = other RNA; note = crPTPN6_7
tctaggtggtaccatggccac

SEQ ID NO: 256 - RNA, 21 nt, synthetic construct
  source 1..21: mol_type = other RNA; note = crPTPN6_8
gcctgcagcagcgtctctgcc

SEQ ID NO: 257 - RNA, 21 nt, synthetic construct
  source 1..21: mol_type = other RNA; note = crPTPN6_9
ttgtgcgtgagagcctcagcc

SEQ ID NO: 258 - RNA, 21 nt, synthetic construct
  source 1..21: mol_type = other RNA; note = crPTPN6_10
gtgctttctgtgctcagtgac

SEQ ID NO: 259 - RNA, 21 nt, synthetic construct
  source 1..21: mol_type = other RNA; note = crPTPN6_11
ggctggtcactgagcacagaa

SEQ ID NO: 260 - RNA, 21 nt, synthetic construct
  source 1..21: mol_type = other RNA; note = crPTPN6_12
ctgtgctcagtgaccagccca

SEQ ID NO: 261 - RNA, 21 nt, synthetic construct
  source 1..21: mol_type = other RNA; note = crPTPN6_13
tgtgctcagtgaccagcccaa

SEQ ID NO: 262 - RNA, 21 nt, synthetic construct
  source 1..21: mol_type = other RNA; note = crPTPN6_14
atgtgggtgaccctgagcggg

SEQ ID NO: 263 - RNA, 21 nt, synthetic construct
  source 1..21: mol_type = other RNA; note = crPTPN6_15
cctcgcacatgaccttgatgt

SEQ ID NO: 264 - RNA, 21 nt, synthetic construct
  source 1..21: mol_type = other RNA; note = crPTPN6_16
gctccccccagggtggacgct

SEQ ID NO: 265 - RNA, 21 nt, synthetic construct
  source 1..21: mol_type = other RNA; note = crPTPN6_17
agcagggtctctgcatccagc

SEQ ID NO: 266 - RNA, 21 nt, synthetic construct
  source 1..21: mol_type = other RNA; note = crPTPN6_18
gagaccttcgacagcctcacg

SEQ ID NO: 267 - RNA, 21 nt, synthetic construct
  source 1..21: mol_type = other RNA; note = crPTPN6_19
gacagcctcacggacctggtg

SEQ ID NO: 268 - RNA, 21 nt, synthetic construct
  source 1..21: mol_type = other RNA; note = crPTPN6_20
aagaagacggggattgaggag

SEQ ID NO: 269 - RNA, 21 nt, synthetic construct
  source 1..21: mol_type = other RNA; note = crPTPN6_21
ttgttcagttccaacactcgg

SEQ ID NO: 270 - RNA, 21 nt, synthetic construct
  source 1..21: mol_type = other RNA; note = crPTPN6_22
gctgtatcctcggactcctgc

SEQ ID NO: 271 - RNA, 21 nt, synthetic construct
  source 1..21: mol_type = other RNA; note = crPTPN6_23
cccacccacatctcagagttt

SEQ ID NO: 272 - RNA, 21 nt, synthetic construct
  source 1..21: mol_type = other RNA; note = crPTPN6_24
cagacgctggtgcaagttctt

SEQ ID NO: 273 - RNA, 21 nt, synthetic construct
  source 1..21: mol_type = other RNA; note = crPTPN6_25
caccagcgtctggaagggcag

SEQ ID NO: 274 - RNA, 21 nt, synthetic construct
  source 1..21: mol_type = other RNA; note = crPTPN6_26
ttctctggccgctgcccttcc

SEQ ID NO: 275 - RNA, 21 nt, synthetic construct
  source 1..21: mol_type = other RNA; note = crPTPN6_27
atgtagttggcattgatgtag

SEQ ID NO: 276 - RNA, 21 nt, synthetic construct
  source 1..21: mol_type = other RNA; note = crPTPN6_28
cgtccagaaccagctgctagg

SEQ ID NO: 277 - RNA, 21 nt, synthetic construct
  source 1..21: mol_type = other RNA; note = crPTPN6_29
tggcagatggcgtggcaggag

SEQ ID NO: 278 - RNA, 21 nt, synthetic construct
  source 1..21: mol_type = other RNA; note = crPTPN6_30
tccacctctcgggtggtcatg

SEQ ID NO: 279 - RNA, 21 nt, synthetic construct
  source 1..21: mol_type = other RNA; note = crPTPN6_31
ctccacctctcgggtggtcat

SEQ ID NO: 280 - RNA, 21 nt, synthetic construct
  source 1..21: mol_type = other RNA; note = crPTPN6_32
ccagaacaaatgcgtcccata

SEQ ID NO: 281 - RNA, 21 nt, synthetic construct
  source 1..21: mol_type = other RNA; note = crPTPN6_33
cagaacaaatgcgtcccatac

SEQ ID NO: 282 - RNA, 21 nt, synthetic construct
  source 1..21: mol_type = other RNA; note = crPTPN6_34
tattcggttgtgtcatgctcc

SEQ ID NO: 283 - RNA, 21 nt, synthetic construct
  source 1..21: mol_type = other RNA; note = crPTPN6_35
caggtctccccgctggacaat

SEQ ID NO: 284 - RNA, 21 nt, synthetic construct
  source 1..21: mol_type = other RNA; note = crPTPN6_36
ctggctcggcccagtcgcaag

SEQ ID NO: 285 - RNA, 21 nt, synthetic construct
  source 1..21: mol_type = other RNA; note = crPTPN6_37
gggagacctgattcgggagat

SEQ ID NO: 286 - RNA, 21 nt, synthetic construct
  source 1..21: mol_type = other RNA; note = crPTPN6_38
ctggaccagatcaaccagcgg

SEQ ID NO: 287 - RNA, 21 nt, synthetic construct
  source 1..21: mol_type = other RNA; note = crPTPN6_39
ctgccgctggttgatctggtc

SEQ ID NO: 288 - RNA, 21 nt, synthetic construct
  source 1..21: mol_type = other RNA; note = crPTPN6_40
cctgccgctggttgatctggt

SEQ ID NO: 289 - RNA, 21 nt, synthetic construct
  source 1..21: mol_type = other RNA; note = crPTPN6_41
gtggagatgttctccatgagc

SEQ ID NO: 290 - RNA, 21 nt, synthetic construct
  source 1..21: mol_type = other RNA; note = crPTPN6_42
tactgcgcctccgtctgcacc

SEQ ID NO: 291 - RNA, 21 nt, synthetic construct
  source 1..21: mol_type = other RNA; note = crPTPN6_43
aatgaactgggcgatggccac

SEQ ID NO: 292 - RNA, 21 nt, synthetic construct
  source 1..21: mol_type = other RNA; note = crPTPN6_44
ttcttagtggtttcaatgaac

SEQ ID NO: 293 - RNA, 21 nt, synthetic construct
  source 1..21: mol_type = other RNA; note = crPTPN6_45
tcccctccatacaggtcatag

SEQ ID NO: 294 - RNA, 21 nt, synthetic construct
  source 1..21: mol_type = other RNA; note = crPTPN6_46
gagtctagtgcagggaccgtg

SEQ ID NO: 295 - RNA, 21 nt, synthetic construct
  source 1..21: mol_type = other RNA; note = crPTPN6_47
cccccctgcacccggctgcag

SEQ ID NO: 296 - RNA, 21 nt, synthetic construct
  source 1..21: mol_type = other RNA; note = crPTPN6_48
tgtctgcagccgggtgcaggg

SEQ ID NO: 297 - RNA, 21 nt, synthetic construct
  source 1..21: mol_type = other RNA; note = crPTPN6_49
tcctccctcttgttcttagtg

SEQ ID NO: 298 - RNA, 21 nt, synthetic construct
  source 1..21: mol_type = other RNA; note = crPTPN6_50
ctcctccctcttgttcttagt

SEQ ID NO: 299 - RNA, 21 nt, synthetic construct
  source 1..21: mol_type = other RNA; note = crPTPN6_51
ttcactttctcctccctcttg

SEQ ID NO: 300 - RNA, 21 nt, synthetic construct
  source 1..21: mol_type = other RNA; note = crPTPN6_52
aggtggatgatggtgccgtcg

SEQ ID NO: 301 - RNA, 21 nt, synthetic construct
  source 1..21: mol_type = other RNA; note = crPTPN6_53
cctgacgctgccttctctagg

SEQ ID NO: 302 - RNA, 21 nt, synthetic construct
  source 1..21: mol_type = other RNA; note = crTIGIT_1
aggccttacctgaggcgaggg

SEQ ID NO: 303 - RNA, 21 nt, synthetic construct
  source 1..21: mol_type = other RNA; note = crTIGIT_2
gtcctccctctagtggctgag

SEQ ID NO: 304 - RNA, 21 nt, synthetic construct
  source 1..21: mol_type = other RNA; note = crTIGIT_3
gggtggcacatctccccatcc

SEQ ID NO: 305 - RNA, 21 nt, synthetic construct
  source 1..21: mol_type = other RNA; note = crTIGIT_4
tgcagagaaaggtggctctat

SEQ ID NO: 306 - RNA, 21 nt, synthetic construct
  source 1..21: mol_type = other RNA; note = crTIGIT_5
taatgctgacttggggtggca

SEQ ID NO: 307 - RNA, 21 nt, synthetic construct
  source 1..21: mol_type = other RNA; note = crTIGIT_6
cctgaggcgaggggagcctgc

SEQ ID NO: 308 - RNA, 21 nt, synthetic construct
  source 1..21: mol_type = other RNA; note = crTIGIT_7
aaggatggggagatgtgccac

SEQ ID NO: 309 - RNA, 21 nt, synthetic construct
  source 1..21: mol_type = other RNA; note = crTIGIT_8
aaggatcgagtggccccaggt

SEQ ID NO: 310 - RNA, 21 nt, synthetic construct
  source 1..21: mol_type = other RNA; note = crTIGIT_9
tgcatctatcacacctaccct

SEQ ID NO: 311 - RNA, 21 nt, synthetic construct
  source 1..21: mol_type = other RNA; note = crTIGIT_10
taggacctccaggaagattct

SEQ ID NO: 312 - RNA, 21 nt, synthetic construct
  source 1..21: mol_type = other RNA; note = crTIGIT_11
ctaggacctccaggaagattc

SEQ ID NO: 313 - RNA, 21 nt, synthetic construct
  source 1..21: mol_type = other RNA; note = crTIGIT_12
ctccagcaggaatacctgagc

SEQ ID NO: 314 - RNA, 21 nt, synthetic construct
  source 1..21: mol_type = other RNA; note = crTIGIT_13
gagccatggccgcgacgctgg

SEQ ID NO: 315 - RNA, 21 nt, synthetic construct
  source 1..21: mol_type = other RNA; note = crTIGIT_14
tagtcaacgcgaccaccacga

SEQ ID NO: 316 - RNA, 21 nt, synthetic construct
  source 1..21: mol_type = other RNA; note = crTIGIT_15
ctagtcaacgcgaccaccacg

SEQ ID NO: 317 - RNA, 21 nt, synthetic construct
  source 1..21: mol_type = other RNA; note = crTIGIT_16
tagtttgtttgtttttagaag

SEQ ID NO: 318 - RNA, 21 nt, synthetic construct
  source 1..21: mol_type = other RNA; note = crTIGIT_17
tttgtttttagaagaaagccc

SEQ ID NO: 319 - RNA, 21 nt, synthetic construct
  source 1..21: mol_type = other RNA; note = crTIGIT_18
tttttagaagaaagccctcag

SEQ ID NO: 320 - RNA, 21 nt, synthetic construct
  source 1..21: mol_type = other RNA; note = crTIGIT_19
tagaagaaagccctcagaatc

SEQ ID NO: 321 - RNA, 21 nt, synthetic construct
  source 1..21: mol_type = other RNA; note = crTIGIT_20
cacagaatggattctgagggc

SEQ ID NO: 322 - RNA, 21 nt, synthetic construct
  source 1..21: mol_type = other RNA; note = crTIGIT_21
ctcctgaggtcaccttccaca

SEQ ID NO: 323 - RNA, 21 nt, synthetic construct
  source 1..21: mol_type = other RNA; note = crTIGIT_22
ctgggggtgagggagcactgg

SEQ ID NO: 324 - RNA, 21 nt, synthetic construct
  source 1..21: mol_type = other RNA; note = crTIGIT_23
tgcctggacacagcttcctgg

SEQ ID NO: 325 - RNA, 21 nt, synthetic construct
  source 1..21: mol_type = other RNA; note = crTIGIT_24
gtcctcttccctaggaatgat

SEQ ID NO: 326 - RNA, 21 nt, synthetic construct
  source 1..21: mol_type = other RNA; note = crTIGIT_25
tgtaactcaggacattgaagt

SEQ ID NO: 327 - RNA, 21 nt, synthetic construct
  source 1..21: mol_type = other RNA; note = crTIGIT_26
aatgtcctgagttacagaagc

SEQ ID NO: 328 - RNA, 21 nt, synthetic construct
  source 1..21: mol_type = other RNA; note = crTIGIT_27
tattgtgcctgtcatcattcc

SEQ ID NO: 329 - RNA, 21 nt, synthetic construct
  source 1..21: mol_type = other RNA; note = crTIGIT_28
tctgcagaaatgttccccgtt

SEQ ID NO: 330 - RNA, 21 nt, synthetic construct
  source 1..21: mol_type = other RNA; note = crTIGIT_29
ctctgcagaaatgttccccgt

SEQ ID NO: 331 - RNA, 21 nt, synthetic construct
  source 1..21: mol_type = other RNA; note = crTIGIT_30
tgccgtggtggaggagaggtg

SEQ ID NO: 332 - RNA, 21 nt, synthetic construct
  source 1..21: mol_type = other RNA; note = crTIGIT_31
tggccatttgtaatgctgact

SEQ ID NO: 333 - RNA, 21 nt, synthetic construct
  source 1..21: mol_type = other RNA; note = crTIM3_1
cttgtaagtagtagcagcagc

SEQ ID NO: 334 - RNA, 21 nt, synthetic construct
  source 1..21: mol_type = other RNA; note = crTIM3_2
caaggatgcttaccaccaggg

SEQ ID NO: 335 - RNA, 21 nt, synthetic construct
  source 1..21: mol_type = other RNA; note = crTIM3_3
taagtagtagcagcagcagca

SEQ ID NO: 336 - RNA, 21 nt, synthetic construct
  source 1..21: mol_type = other RNA; note = crTIM3_4
ccaccaggggacatggcccag

SEQ ID NO: 337 - RNA, 21 nt, synthetic construct
  source 1..21: mol_type = other RNA; note = crTIM3_5
aatgtggcaacgtggtgctca

SEQ ID NO: 338 - RNA, 21 nt, synthetic construct
  source 1..21: mol_type = other RNA; note = crTIM3_6
tcttctgcaagctccatgttt

SEQ ID NO: 339 - RNA, 21 nt, synthetic construct
  source 1..21: mol_type = other RNA; note = crTIM3_7
gccccagcagacgggcacgag

SEQ ID NO: 340 - RNA, 21 nt, synthetic construct
  source 1..21: mol_type = other RNA; note = crTIM3_8
atcagtcctgagcaccacgtt

SEQ ID NO: 341 - RNA, 21 nt, synthetic construct
  source 1..21: mol_type = other RNA; note = crTIM3_9
catcagtcctgagcaccacgt

SEQ ID NO: 342 - RNA, 21 nt, synthetic construct
  source 1..21: mol_type = other RNA; note = crTIM3_10
gccagtatctggatgtccaat

SEQ ID NO: 343 - RNA, 21 nt, synthetic construct
  source 1..21: mol_type = other RNA; note = crTIM3_11
cggaaatccccatttagccag

SEQ ID NO: 344 - RNA, 21 nt, synthetic construct
  source 1..21: mol_type = other RNA; note = crTIM3_12
gcggaaatccccatttagcca

SEQ ID NO: 345 - RNA, 21 nt, synthetic construct
  source 1..21: mol_type = other RNA; note = crTIM3_13
cgcaaaggagatgtgtccctg

SEQ ID NO: 346 - RNA, 21 nt, synthetic construct
  source 1..21: mol_type = other RNA; note = crTIM3_14
gatccggcagcagtagatccc

SEQ ID NO: 347 - RNA, 21 nt, synthetic construct
  source 1..21: mol_type = other RNA; note = crTIM3_15
tcatcattcattatgcctggg

SEQ ID NO: 348 - RNA, 21 nt, synthetic construct
  source 1..21: mol_type = other RNA; note = crTIM3_16
cttctgcaagctccatgtttt

SEQ ID NO: 349 - RNA, 21 nt, synthetic construct
  source 1..21: mol_type = other RNA; note = crTIM3_17
aggttaaatttttcatcattc

SEQ ID NO: 350 - RNA, 21 nt, synthetic construct
  source 1..21: mol_type = other RNA; note = crTIM3_18
atgaccaacttcaggttaaat

SEQ ID NO: 351 - RNA, 21 nt, synthetic construct
  source 1..21: mol_type = other RNA; note = crTIM3_19
acctgaagttggtcatcaaac

SEQ ID NO: 352 - RNA, 21 nt, synthetic construct
  source 1..21: mol_type = other RNA; note = crTIM3_20
tgttgtttctgacattagcca

SEQ ID NO: 353 - RNA, 21 nt, synthetic construct
  source 1..21: mol_type = other RNA; note = crTIM3_21
tgacattagccaaggtcaccc

SEQ ID NO: 354 - RNA, 21 nt, synthetic construct
  source 1..21: mol_type = other RNA; note = crTIM3_22
gaaaggctgcagtgaagtctc

SEQ ID NO: 355 - RNA, 21 nt, synthetic construct
  source 1..21: mol_type = other RNA; note = crTIM3_23
actgcagcctttccaaggatg

SEQ ID NO: 356 - RNA, 21 nt, synthetic construct
  source 1..21: mol_type = other RNA; note = crTIM3_24
ccaaggatgcttaccaccagg

SEQ ID NO: 357 - RNA, 21 nt, synthetic construct
  source 1..21: mol_type = other RNA; note = crTIM3_25
cacatcttccctttgactgtg

SEQ ID NO: 358 - RNA, 21 nt, synthetic construct
  source 1..21: mol_type = other RNA; note = crTIM3_26
tatagcagagacacagacact

SEQ ID NO: 359 - RNA, 21 nt, synthetic construct
  source 1..21: mol_type = other RNA; note = crTIM3_27
tatcagggaggctccccagtg

SEQ ID NO: 360 - RNA, 21 nt, synthetic construct
  source 1..21: mol_type = other RNA; note = crTIM3_28
ctgttagatttatatcaggga

SEQ ID NO: 361 - RNA, 21 nt, synthetic construct
  source 1..21: mol_type = other RNA; note = crTIM3_29
tgtttccatagcaaatatcca

SEQ ID NO: 362 - RNA, 21 nt, synthetic construct
  source 1..21: mol_type = other RNA; note = crTIM3_30
catagcaaatatccacattgg

SEQ ID NO: 363 - RNA, 21 nt, synthetic construct
  source 1..21: mol_type = other RNA; note = crTIM3_31
cgggactctggagcaaccatc

SEQ ID NO: 364 - RNA, 21 nt, synthetic construct
  source 1..21: mol_type = other RNA; note = crTIM3_32
aaaattaaagcgccgaagata

SEQ ID NO: 365 - RNA, 21 nt, synthetic construct
  source 1..21: mol_type = other RNA; note = crTIM3_33
catttgaaaattaaagcgccg

SEQ ID NO: 366 - RNA, 21 nt, synthetic construct
  source 1..21: mol_type = other RNA; note = crTIM3_34
tgtttcccccttactagggta

SEQ ID NO: 367 - RNA, 21 nt, synthetic construct
  source 1..21: mol_type = other RNA; note = crTIM3_35
gtttcccccttactagggtat

SEQ ID NO: 368 - RNA, 21 nt, synthetic construct
  source 1..21: mol_type = other RNA; note = crTIM3_36
gactgtgtcctgctgctgctg

SEQ ID NO: 369 - RNA, 21 nt, synthetic construct
  source 1..21: mol_type = other RNA; note = crTIM3_37
ccccttactagggtattctca

SEQ ID NO: 370 - RNA, 21 nt, synthetic construct
  source 1..21: mol_type = other RNA; note = crTIM3_38
ctagggtattctcatagcaaa

SEQ ID NO: 371 - RNA, 21 nt, synthetic construct
  source 1..21: mol_type = other RNA; note = crTIM3_39
aattctgtatcttctctttgc

SEQ ID NO: 372 - RNA, 21 nt, synthetic construct
  source 1..21: mol_type = other RNA; note = crTIM3_40
atttccacagcctcatctctt

SEQ ID NO: 373 - RNA, 21 nt, synthetic construct
  source 1..21: mol_type = other RNA; note = crTIM3_41
tttccacagcctcatctcttt

SEQ ID NO: 374 - RNA, 21 nt, synthetic construct
  source 1..21: mol_type = other RNA; note = crTIM3_42
cacagcctcatctctttggcc

SEQ ID NO: 375 - RNA, 21 nt, synthetic construct
  source 1..21: mol_type = other RNA; note = crTIM3_43
gccaacctccctccctcagga

SEQ ID NO: 376 - RNA, 21 nt, synthetic construct
  source 1..21: mol_type = other RNA; note = crTIM3_44
ccaatcctgagggagggaggt

SEQ ID NO: 377 - RNA, 21 nt, synthetic construct
  source 1..21: mol_type = other RNA; note = crTIM3_45
cttctgagcgaattccctctg

SEQ ID NO: 378 - RNA, 21 nt, synthetic construct
  source 1..21: mol_type = other RNA; note = crTIM3_46
atatacgttctcttcaatggt

SEQ ID NO: 379 - RNA, 21 nt, synthetic construct
  source 1..21: mol_type = other RNA; note = crTIM3_47
gggttgtcgctttgcaatgcc

SEQ ID NO: 380 - RNA, 21 nt, synthetic construct
  source 1..21: mol_type = other RNA; note = crTIM3_48
ggttgtcgctttgcaatgcca

SEQ ID NO: 381 - RNA, 21 nt, synthetic construct
  source 1..21: mol_type = other RNA; note = crTIM3_49
tctctctatgcagggtcctca

SEQ ID NO: 382 - RNA, 21 nt, synthetic construct
  source 1..21: mol_type = other RNA; note = crTIM3_50
tacaccccagccgccccaggg

SEQ ID NO: 383 - RNA, 21 nt, synthetic construct
  source 1..21: mol_type = other RNA; note = crTIM3_51
ccccagcagacgggcacgagg

SEQ ID NO: 384 - DNA, 21 bp, synthetic construct
  source 1..21: mol_type = other DNA; note = crAAVS1
ttaggatggccttctccgacg

SEQ ID NO: 385 - DNA, 20 bp, synthetic construct
  source 1..20: mol_type = other DNA; note = crCD247_1
tggggaggtagctgcagaat

SEQ ID NO: 386 - DNA, 20 bp, synthetic construct
  source 1..20: mol_type = other DNA; note = crCD247_2
tggggaggtagctgcagaat

SEQ ID NO: 387 - DNA, 20 bp, synthetic construct
  source 1..20: mol_type = other DNA; note = crCD247_3
tggggatgtgttctcgtcac

SEQ ID NO: 388 - DNA, 20 bp, synthetic construct
  source 1..20: mol_type = other DNA; note = crCD247_4
ggtagcacagggaggagaga

SEQ ID NO: 389 - DNA, 20 bp, synthetic construct
  source 1..20: mol_type = other DNA; note = crCD247_5
ttagttgccaaggagcggag

SEQ ID NO: 390 - DNA, 20 bp, synthetic construct
  source 1..20: mol_type = other DNA; note = crCD247_6
gcccttcctccaactttcca

SEQ ID NO: 391 - DNA, 20 bp, synthetic construct
  source 1..20: mol_type = other DNA; note = crCD247_7
ggtagcacagggaggagaga

SEQ ID NO: 392 - DNA, 20 bp, synthetic construct
  source 1..20: mol_type = other DNA; note = crCD247_8
cgtgtctggaggaccaagag

SEQ ID NO: 393 - DNA, 20 bp, synthetic construct
  source 1..20: mol_type = other DNA; note = crCD247_9
ctggttgtgggcagagaagt

SEQ ID NO: 394 - DNA, 20 bp, synthetic construct
  source 1..20: mol_type = other DNA; note = crCD247_10
tgcagctgggatgagaagtg

SEQ ID NO: 395 - DNA, 20 bp, synthetic construct
  source 1..20: mol_type = other DNA; note = crCD247_11
tgcagctgggatgagaagtg

SEQ ID NO: 396 - DNA, 20 bp, synthetic construct
  source 1..20: mol_type = other DNA; note = crCD247_12
ggcctcaccttactctgcag

SEQ ID NO: 397 - DNA, 20 bp, synthetic construct
  source 1..20: mol_type = other DNA; note = crCD247_13
ggcctcaccttactctgcag

SEQ ID NO: 398 - DNA, 20 bp, synthetic construct
  source 1..20: mol_type = other DNA; note = crCD247_14
ggcctcaccttactctgcag

SEQ ID NO: 399 - DNA, 20 bp, synthetic construct
  source 1..20: mol_type = other DNA; note = crCD247_15
taaacccaagactctggcgg

SEQ ID NO: 400 - DNA, 20 bp, synthetic construct
  source 1..20: mol_type = other DNA; note = crCD247_16
acagcacccatctaccaacg

SEQ ID NO: 401 - DNA, 20 bp, synthetic construct
  source 1..20: mol_type = other DNA; note = crCD247_17
acagcacccatctaccaacg

SEQ ID NO: 402 - DNA, 20 bp, synthetic construct
  source 1..20: mol_type = other DNA; note = crCD247_18
acagcacccatctaccaacg

SEQ ID NO: 403 - DNA, 20 bp, synthetic construct
  source 1..20: mol_type = other DNA; note = crCD247_19
cagggggattattcctgggc

SEQ ID NO: 404 - DNA, 20 bp, synthetic construct
  source 1..20: mol_type = other DNA; note = crCD247_20
tatggcgccctttgagacag

SEQ ID NO: 405 - DNA, 20 bp, synthetic construct
  source 1..20: mol_type = other DNA; note = crCD247_21
gcccctgcccctctttttat

SEQ ID NO: 406 - DNA, 20 bp, synthetic construct
  source 1..20: mol_type = other DNA; note = crCD247_22
gcccctgcccctctttttat

SEQ ID NO: 407 - DNA, 20 bp, synthetic construct
  source 1..20: mol_type = other DNA; note = crCD247_23
tggttgcagagtgagctgag

SEQ ID NO: 408 - DNA, 20 bp, synthetic construct
  source 1..20: mol_type = other DNA; note = crCD247_24
tggttgcagagtgagctgag

SEQ ID NO: 409 - DNA, 20 bp, synthetic construct
  source 1..20: mol_type = other DNA; note = crCD247_25
gcccctgcccctctttttat

SEQ ID NO: 410 - DNA, 20 bp, synthetic construct
  source 1..20: mol_type = other DNA; note = crCD247_26
ggtagcacagggaggagaga

SEQ ID NO: 411 - DNA, 20 bp, synthetic construct
  source 1..20: mol_type = other DNA; note = crCTLA4_1
atcatgtaggttgccgcaca

SEQ ID NO: 412 - DNA, 20 bp, synthetic construct
  source 1..20: mol_type = other DNA; note = crCTLA4_2
tcactgcctttgactgctga

SEQ ID NO: 413 - DNA, 20 bp, synthetic construct
  source 1..20: mol_type = other DNA; note = crCTLA4_3
aaatctgggttccgttgcct

SEQ ID NO: 414 - DNA, 20 bp, synthetic construct
  source 1..20: mol_type = other DNA; note = crCTLA4_4
ggccatgaaggagcatgagt

SEQ ID NO: 415 - DNA, 21 bp, synthetic construct
  source 1..21: mol_type = other DNA; note = crCTLA4_5
agtccttgattctgtgtgggt

SEQ ID NO: 416 - DNA, 21 bp, synthetic construct
  source 1..21: mol_type = other DNA; note = crCTLA4_6
cctcctccatcttcatgctcc

SEQ ID NO: 417 - DNA, 20 bp, synthetic construct
  source 1..20: mol_type = other DNA; note = crCTLA4_7
aagctagaaggcagaagggc

SEQ ID NO: 418 - DNA, 20 bp, synthetic construct
  source 1..20: mol_type = other DNA; note = crCTLA4_8
aagctagaaggcagaagggc

SEQ ID NO: 419 - DNA, 20 bp, synthetic construct
  source 1..20: mol_type = other DNA; note = crCTLA4_9
ggccatgaaggagcatgagt

SEQ ID NO: 420 - DNA, 20 bp, synthetic construct
  source 1..20: mol_type = other DNA; note = crCTLA4_10
ggccatgaaggagcatgagt

SEQ ID NO: 421 - DNA, 20 bp, synthetic construct
  source 1..20: mol_type = other DNA; note = crCTLA4_11
catgctagcaatgcacgtgg

SEQ ID NO: 422 - DNA, 20 bp, synthetic construct
  source 1..20: mol_type = other DNA; note = crCTLA4_12
catgctagcaatgcacgtgg

SEQ ID NO: 423 - DNA, 20 bp, synthetic construct
  source 1..20: mol_type = other DNA; note = crCTLA4_13
catcgccagctttgtgtgtg

SEQ ID NO: 424 - DNA, 21 bp, synthetic construct
  source 1..21: mol_type = other DNA; note = crCTLA4_14
agtccttgattctgtgtgggt

SEQ ID NO: 425 - DNA, 20 bp, synthetic construct
  source 1..20: mol_type = other DNA; note = crCTLA4_15
aggtgactgaagtctgtgcg

SEQ ID NO: 426 - DNA, 20 bp, synthetic construct
  source 1..20: mol_type = other DNA; note = crCTLA4_16
aggtgactgaagtctgtgcg

SEQ ID NO: 427 - DNA, 20 bp, synthetic construct
  source 1..20: mol_type = other DNA; note = crCTLA4_17
aggtgactgaagtctgtgcg

SEQ ID NO: 428 - DNA, 20 bp, synthetic construct
  source 1..20: mol_type = other DNA; note = crCTLA4_18
catctgcaaggtggagctca

SEQ ID NO: 429 - DNA, 20 bp, synthetic construct
  source 1..20: mol_type = other DNA; note = crCTLA4_19
tctgcaaggtggagctcatg

SEQ ID NO: 430 - DNA, 21 bp, synthetic construct
  source 1..21: mol_type = other DNA; note = crCTLA4_20
gcaatttaggggtggacctca

SEQ ID NO: 431 - DNA, 21 bp, synthetic construct
  source 1..21: mol_type = other DNA; note = crCTLA4_21
gcaatttaggggtggacctca

SEQ ID NO: 432 - DNA, 20 bp, synthetic construct
  source 1..20: mol_type = other DNA; note = crCTLA4_22
aatgttggggagtagagccc

SEQ ID NO: 433 - DNA, 22 bp, synthetic construct
  source 1..22: mol_type = other DNA; note = crCTLA4_23
caatgttggggagtagagccct

SEQ ID NO: 434 - DNA, 20 bp, synthetic construct
  source 1..20: mol_type = other DNA; note = crCTLA4_24
atgttggggagtagagccct

SEQ ID NO: 435 - DNA, 21 bp, synthetic construct
  source 1..21: mol_type = other DNA; note = crCTLA4_25
agtccttgattctgtgtgggt

SEQ ID NO: 436 - DNA, 20 bp, synthetic construct
  source 1..20: mol_type = other DNA; note = crCTLA4_26
atgttggggagtagagccct

SEQ ID NO: 437 - DNA, 20 bp, synthetic construct
  source 1..20: mol_type = other DNA; note = crCTLA4_27
atgttggggagtagagccct

SEQ ID NO: 438 - DNA, 20 bp, synthetic construct
  source 1..20: mol_type = other DNA; note = crCTLA4_28
atgttggggagtagagccct

SEQ ID NO: 439 - DNA, 20 bp, synthetic construct
  source 1..20: mol_type = other DNA; note = crCTLA4_29
atgttggggagtagagccct

SEQ ID NO: 440 - DNA, 22 bp, synthetic construct
  source 1..22: mol_type = other DNA; note = crCTLA4_30
agggacccaatatgtgttgagt

SEQ ID NO: 441 - DNA, 22 bp, synthetic construct
  source 1..22: mol_type = other DNA; note = crCTLA4_31
agggacccaatatgtgttgagt

SEQ ID NO: 442 - DNA, 22 bp, synthetic construct
  source 1..22: mol_type = other DNA; note = crCTLA4_32
agggacccaatatgtgttgagt

SEQ ID NO: 443 - DNA, 20 bp, synthetic construct
  source 1..20: mol_type = other DNA; note = crCTLA4_33
tggttagaagtggcttccgt

SEQ ID NO: 444 - DNA, 20 bp, synthetic construct
  source 1..20: mol_type = other DNA; note = crCTLA4_34
tggttagaagtggcttccgt

SEQ ID NO: 445 - DNA, 20 bp, synthetic construct
  source 1..20: mol_type = other DNA; note = crCTLA4_35
tggttagaagtggcttccgt

SEQ ID NO: 446 - DNA, 20 bp, synthetic construct
  source 1..20: mol_type = other DNA; note = crCTLA4_36
ccctcttacaacaggggtct

SEQ ID NO: 447 - DNA, 20 bp, synthetic construct
  source 1..20: mol_type = other DNA; note = crCTLA4_37
ccctcttacaacaggggtct

SEQ ID NO: 448 - DNA, 20 bp, synthetic construct
  source 1..20: mol_type = other DNA; note = crCTLA4_38
tgaagacctgaacaccgctc

SEQ ID NO: 449 - DNA, 20 bp, synthetic construct
  source 1..20: mol_type = other DNA; note = crCTLA4_39
tgaagacctgaacaccgctc

SEQ ID NO: 450 - DNA, 20 bp, synthetic construct
  source 1..20: mol_type = other DNA; note = crCTLA4_40
aagctagaaggcagaagggc

SEQ ID NO: 451 - DNA, 20 bp, synthetic construct
  source 1..20: mol_type = other DNA; note = crCTLA4_41
aagctagaaggcagaagggc

SEQ ID NO: 452 - DNA, 20 bp, synthetic construct
  source 1..20: mol_type = other DNA; note = crLAG3_1
tagtgaagcctctccagcca

SEQ ID NO: 453 - DNA, 20 bp, synthetic construct
  source 1..20: mol_type = other DNA; note = crLAG3_2
ccaagtgagtgcagggtgat

SEQ ID NO: 454 - DNA, 20 bp, synthetic construct
  source 1..20: mol_type = other DNA; note = crLAG3_3
tggggaagctgctttgtgag

SEQ ID NO: 455 - DNA, 22 bp, synthetic construct
  source 1..22: mol_type = other DNA; note = crLAG3_4
ctggatccctggggaagctgct

SEQ ID NO: 456 - DNA, 20 bp, synthetic construct
  source 1..20: mol_type = other DNA; note = crLAG3_5
ccaagtgagtgcagggtgat

SEQ ID NO: 457 - DNA, 22 bp, synthetic construct
  source 1..22: mol_type = other DNA; note = crLAG3_6
ccttttggagggctcagcgctg

SEQ ID NO: 458 - DNA, 20 bp, synthetic construct
  source 1..20: mol_type = other DNA; note = crLAG3_7
ctgagatggggagagggtga

SEQ ID NO: 459 - DNA, 20 bp, synthetic construct
  source 1..20: mol_type = other DNA; note = crLAG3_8
tccagtgggctgatgaagtc

SEQ ID NO: 460 - DNA, 20 bp, synthetic construct
  source 1..20: mol_type = other DNA; note = crLAG3_9
tccagtgggctgatgaagtc

SEQ ID NO: 461 - DNA, 20 bp, synthetic construct
  source 1..20: mol_type = other DNA; note = crLAG3_10
ggatctctcagagcctccga

SEQ ID NO: 462 - DNA, 20 bp, synthetic construct
  source 1..20: mol_type = other DNA; note = crLAG3_11
ggatctctcagagcctccga

SEQ ID NO: 463 - DNA, 22 bp, synthetic construct
  source 1..22: mol_type = other DNA; note = crLAG3_12
gcccagcctctgtgcattggtt

SEQ ID NO: 464 - DNA, 22 bp, synthetic construct
  source 1..22: mol_type = other DNA; note = crLAG3_13
gcccagcctctgtgcattggtt

SEQ ID NO: 465 - DNA, 22 bp, synthetic construct
  source 1..22: mol_type = other DNA; note = crLAG3_14
gcccagcctctgtgcattggtt

SEQ ID NO: 466 - DNA, 20 bp, synthetic construct
  source 1..20: mol_type = other DNA; note = crLAG3_15
cttcctcttcctgccccaag

SEQ ID NO: 467 - DNA, 20 bp, synthetic construct
  source 1..20: mol_type = other DNA; note = crLAG3_16
tgagccagaccatctcctga

SEQ ID NO: 468 - DNA, 22 bp, synthetic construct
  source 1..22: mol_type = other DNA; note = crLAG3_17
ccttttggagggctcagcgctg

SEQ ID NO: 469 - DNA, 20 bp, synthetic construct
  source 1..20: mol_type = other DNA; note = crLAG3_18
tgagccagaccatctcctga

SEQ ID NO: 470 - DNA, 20 bp, synthetic construct
  source 1..20: mol_type = other DNA; note = crLAG3_19
tgagccagaccatctcctga

SEQ ID NO: 471 - DNA, 22 bp, synthetic construct
  source 1..22: mol_type = other DNA; note = crLAG3_20
gtctggagcccccaactccctt

SEQ ID NO: 472 - DNA, 22 bp, synthetic construct
  source 1..22: mol_type = other DNA; note = crLAG3_21
gtctggagcccccaactccctt

SEQ ID NO: 473 - DNA, 20 bp, synthetic construct
  source 1..20: mol_type = other DNA; note = crLAG3_22
gacccggtctttcctcactg

SEQ ID NO: 474 - DNA, 20 bp, synthetic construct
  source 1..20: mol_type = other DNA; note = crLAG3_23
gacccggtctttcctcactg

SEQ ID NO: 475 - DNA, 20 bp, synthetic construct
  source 1..20: mol_type = other DNA; note = crLAG3_24
gacccggtctttcctcactg

SEQ ID NO: 476 - DNA, 20 bp, synthetic construct
  source 1..20: mol_type = other DNA; note = crLAG3_25
tggcgactttacccttcgac

SEQ ID NO: 477 - DNA, 20 bp, synthetic construct
  source 1..20: mol_type = other DNA; note = crLAG3_26
tggcgactttacccttcgac

SEQ ID NO: 478 - DNA, 20 bp, synthetic construct
  source 1..20: mol_type = other DNA; note = crLAG3_27
ccaagtgagtgcagggtgat

SEQ ID NO: 479 - DNA, 22 bp, synthetic construct
  source 1..22: mol_type = other DNA; note = crLAG3_28
ccttttggagggctcagcgctg

SEQ ID NO: 480 - DNA, 20 bp, synthetic construct
  source 1..20: mol_type = other DNA; note = crLAG3_29
ccaagtgagtgcagggtgat

SEQ ID NO: 481 - DNA, 20 bp, synthetic construct
  source 1..20: mol_type = other DNA; note = crLAG3_30
ccaagtgagtgcagggtgat

SEQ ID NO: 482 - DNA, 20 bp, synthetic construct
  source 1..20: mol_type = other DNA; note = crLAG3_31
ccaagtgagtgcagggtgat

SEQ ID NO: 483 - DNA, 20 bp, synthetic construct
  source 1..20: mol_type = other DNA; note = crLAG3_32
ccaagtgagtgcagggtgat

SEQ ID NO: 484 - DNA, 20 bp, synthetic construct
  source 1..20: mol_type = other DNA; note = crLAG3_33
tcctttgggtcacctggatc

SEQ ID NO: 485 - DNA, 20 bp, synthetic construct
  source 1..20: mol_type = other DNA; note = crLAG3_34
tcctttgggtcacctggatc

SEQ ID NO: 486 - DNA, 20 bp, synthetic construct
  source 1..20: mol_type = other DNA; note = crLAG3_35
agaacgctttgtgtggagct

SEQ ID NO: 487 - DNA, 20 bp, synthetic construct
  source 1..20: mol_type = other DNA; note = crLAG3_36
ttcctgcaccctgtttctcc

SEQ ID NO: 488 - DNA, 20 bp, synthetic construct
  source 1..20: mol_type = other DNA; note = crLAG3_37
agaacgctttgtgtggagct

SEQ ID NO: 489 - DNA, 22 bp, synthetic construct
  source 1..22: mol_type = other DNA; note = crLAG3_38
ctggatccctggggaagctgct

SEQ ID NO: 490 - DNA, 20 bp, synthetic construct
  source 1..20: mol_type = other DNA; note = crLAG3_39
tttctcaggaccttggctgg

SEQ ID NO: 491 - DNA, 20 bp, synthetic construct
  source 1..20: mol_type = other DNA; note = crLAG3_40
ctttcccagccttggcaatg

SEQ ID NO: 492 - DNA, 20 bp, synthetic construct
  source 1..20: mol_type = other DNA; note = crLAG3_41
gctgaatgaccctgggacaa

SEQ ID NO: 493 - DNA, 20 bp, synthetic construct
  source 1..20: mol_type = other DNA; note = crLAG3_42
gctgaatgaccctgggacaa

SEQ ID NO: 494 - DNA, 22 bp, synthetic construct
  source 1..22: mol_type = other DNA; note = crLAG3_43
ccataggtgcccaacgctctgg

SEQ ID NO: 495 - DNA, 22 bp, synthetic construct
  source 1..22: mol_type = other DNA; note = crLAG3_44
ccataggtgcccaacgctctgg

SEQ ID NO: 496 - DNA, 22 bp, synthetic construct
  source 1..22: mol_type = other DNA; note = crLAG3_45
ccataggtgcccaacgctctgg

SEQ ID NO: 497 - DNA, 20 bp, synthetic construct
  source 1..20: mol_type = other DNA; note = crLAG3_46
ggcctctcttttgctcacct

SEQ ID NO: 498 - DNA, 22 bp, synthetic construct
  source 1..22: mol_type = other DNA; note = crLAG3_47
ccataggtgcccaacgctctgg

SEQ ID NO: 499 - DNA, 22 bp, synthetic construct
  source 1..22: mol_type = other DNA; note = crLAG3_48
ccataggtgcccaacgctctgg

SEQ ID NO: 500 - DNA, 22 bp, synthetic construct
  source 1..22: mol_type = other DNA; note = crLAG3_49
ccataggtgcccaacgctctgg

SEQ ID NO: 501 - DNA, 22 bp, synthetic construct
  source 1..22: mol_type = other DNA; note = crLAG3_50
ccataggtgcccaacgctctgg

SEQ ID NO: 502 - DNA, 20 bp, synthetic construct
  source 1..20: mol_type = other DNA; note = crLAG3_51
catccttctcctccttccgc

SEQ ID NO: 503 - DNA, 20 bp, synthetic construct
  source 1..20: mol_type = other DNA; note = crLAG3_52
catccttctcctccttccgc

SEQ ID NO: 504 - DNA, 20 bp, synthetic construct
  source 1..20: mol_type = other DNA; note = crLAG3_53
catccttctcctccttccgc

SEQ ID NO: 505 - DNA, 20 bp, synthetic construct
  source 1..20: mol_type = other DNA; note = crLAG3_54
gacggttggtggtcaagaga

SEQ ID NO: 506 - DNA, 18 bp, synthetic construct
  source 1..18: mol_type = other DNA; note = crLAG3_55
cgctacacggtgctgagc

SEQ ID NO: 507 - DNA, 20 bp, synthetic construct
  source 1..20: mol_type = other DNA; note = crLAG3_56
ctgagatggggagagggtga

SEQ ID NO: 508 - DNA, 22 bp, synthetic construct
  source 1..22: mol_type = other DNA; note = crPDCD1_1
tctctcagactccccagacagg

SEQ ID NO: 509 - DNA, 22 bp, synthetic construct
  source 1..22: mol_type = other DNA; note = crPDCD1_2
ctaagtccctgatgaaggcccc

SEQ ID NO: 510 - DNA, 22 bp, synthetic construct
  source 1..22: mol_type = other DNA; note = crPDCD1_3
gctgactccctctccctttctc

SEQ ID NO: 511 - DNA, 22 bp, synthetic construct
  source 1..22: mol_type = other DNA; note = crPDCD1_4
tctctgtggactatggggagct

SEQ ID NO: 512 - DNA, 22 bp, synthetic construct
  source 1..22: mol_type = other DNA; note = crPDCD1_5
ctgcagcttctccaacacatcg

SEQ ID NO: 513 - DNA, 20 bp, synthetic construct
  source 1..20: mol_type = other DNA; note = crPDCD1_6
gatgtggaggaagagggggc

SEQ ID NO: 514 - DNA, 22 bp, synthetic construct
  source 1..22: mol_type = other DNA; note = crPDCD1_7
ctgcagcttctccaacacatcg

SEQ ID NO: 515 - DNA, 22 bp, synthetic construct
  source 1..22: mol_type = other DNA; note = crPDCD1_8
ctgcagcttctccaacacatcg

SEQ ID NO: 516 - DNA, 22 bp, synthetic construct
  source 1..22: mol_type = other DNA; note = crPDCD1_9
ctgcagcttctccaacacatcg

SEQ ID NO: 517 - DNA, 22 bp, synthetic construct
  source 1..22: mol_type = other DNA; note = crPDCD1_10
ctgcagcttctccaacacatcg

SEQ ID NO: 518 - DNA, 20 bp, synthetic construct
  source 1..20: mol_type = other DNA; note = crPDCD1_11
gcgtgacttccacatgagcg

SEQ ID NO: 519 - DNA, 20 bp, synthetic construct
  source 1..20: mol_type = other DNA; note = crPDCD1_12
gcgtgacttccacatgagcg

SEQ ID NO: 520 - DNA, 20 bp, synthetic construct
  source 1..20: mol_type = other DNA; note = crPDCD1_13
gcgtgacttccacatgagcg

SEQ ID NO: 521 - DNA, 21 bp, synthetic construct
  source 1..21: mol_type = other DNA; note = crPDCD1_14
ctctagtctgccctcacccct

SEQ ID NO: 522 - DNA, 21 bp, synthetic construct
  source 1..21: mol_type = other DNA; note = crPDCD1_15
ctctagtctgccctcacccct

SEQ ID NO: 523 - DNA, 20 bp, synthetic construct
  source 1..20: mol_type = other DNA; note = crPDCD1_16
gatgtggaggaagagggggc

SEQ ID NO: 524 - DNA, 21 bp, synthetic construct
  source 1..21: mol_type = other DNA; note = crPDCD1_17
ctctagtctgccctcacccct

SEQ ID NO: 525 - DNA, 21 bp, synthetic construct
  source 1..21: mol_type = other DNA; note = crPDCD1_18
ctctagtctgccctcacccct

SEQ ID NO: 526 - DNA, 21 bp, synthetic construct
  source 1..21: mol_type = other DNA; note = crPDCD1_19
ctctagtctgccctcacccct

SEQ ID NO: 527 - DNA, 22 bp, synthetic construct
  source 1..22: mol_type = other DNA; note = crPDCD1_20
cagctcagggtaagcagctcat

SEQ ID NO: 528 - DNA, 22 bp, synthetic construct
  source 1..22: mol_type = other DNA; note = crPDCD1_21
cagctcagggtaagcagctcat

SEQ ID NO: 529 - DNA, 22 bp, synthetic construct
  source 1..22: mol_type = other DNA; note = crPDCD1_22
gctgactccctctccctttctc

SEQ ID NO: 530 - DNA, 22 bp, synthetic construct
  source 1..22: mol_type = other DNA; note = crPDCD1_23
tctctgtggactatggggagct

SEQ ID NO: 531 - DNA, 20 bp, synthetic construct
  source 1..20: mol_type = other DNA; note = crPDCD1_24
gatgtggaggaagagggggc

SEQ ID NO: 532 - DNA, 22 bp, synthetic construct
  source 1..22: mol_type = other DNA; note = crPDCD1_25
gccaccattgtctttcctagcg

SEQ ID NO: 533 - DNA, 20 bp, synthetic construct
  source 1..20: mol_type = other DNA; note = crPDCD1_26
gatgtggaggaagagggggc

SEQ ID NO: 534 - DNA, 22 bp, synthetic construct
  source 1..22: mol_type = other DNA; note = crPDCD1_27
tctctcagactccccagacagg

SEQ ID NO: 535 - DNA, 22 bp, synthetic construct
  source 1..22: mol_type = other DNA; note = crPDCD1_28
tctctcagactccccagacagg

SEQ ID NO: 536 - DNA, 22 bp, synthetic construct
  source 1..22: mol_type = other DNA; note = crPDCD1_29
tctctcagactccccagacagg

SEQ ID NO: 537 - DNA, 22 bp, synthetic construct
  source 1..22: mol_type = other DNA; note = crPDCD1_30
tctctcagactccccagacagg

SEQ ID NO: 538 - DNA, 21 bp, synthetic construct
  source 1..21: mol_type = other DNA; note = crPTPN1_1
tggtgtctgtcttctgtcagc

SEQ ID NO: 539 - DNA, 22 bp, synthetic construct
  source 1..22: mol_type = other DNA; note = crPTPN1_2
cgaaatgcaggcagcaagctat

SEQ ID NO: 540 - DNA, 21 bp, synthetic construct
  source 1..21: mol_type = other DNA; note = crPTPN1_3
ctctgggaaagaagcagagaa

SEQ ID NO: 541 - DNA, 24 bp, synthetic construct
  source 1..24: mol_type = other DNA; note = crPTPN11_4
ttctgtctacctctgtatgtttgc

SEQ ID NO: 542 - DNA, 21 bp, synthetic construct
  source 1..21: mol_type = other DNA; note = crPTPN11_5
cttggactaggctggggagta

SEQ ID NO: 543 - DNA, 23 bp, synthetic construct
  source 1..23: mol_type = other DNA; note = crPTPN11_6
aggacgtcagtttcaagtctctc

SEQ ID NO: 544 - DNA, 23 bp, synthetic construct
  source 1..23: mol_type = other DNA; note = crPTPN11_7
tccaagcatggttttaccacttc

SEQ ID NO: 545 - DNA, 23 bp, synthetic construct
  source 1..23: mol_type = other DNA; note = crPTPN11_8
cgcacacaattctgaacatttcc

SEQ ID NO: 546 - DNA, 25 bp, synthetic construct
  source 1..25: mol_type = other DNA; note = crPTPN11_9
cccttggaggaatgtgtctactttt

SEQ ID NO: 547 - DNA, 23 bp, synthetic construct
  source 1..23: mol_type = other DNA; note = crPTPN11_10
gaacaaaatctccagggtggctc

SEQ ID NO: 548 - DNA, 21 bp, synthetic construct
  source 1..21: mol_type = other DNA; note = crPTPN6_1
ctctactcctgcaccgactgg

SEQ ID NO: 549 - DNA, 20 bp, synthetic construct
  source 1..20: mol_type = other DNA; note = crPTPN6_2
gggggatcaggtgacccata

SEQ ID NO: 550 - DNA, 20 bp, synthetic construct
  source 1..20: mol_type = other DNA; note = crPTPN6_3
cccgatggatgccctctttg

SEQ ID NO: 551 - DNA, 20 bp, synthetic construct
  source 1..20: mol_type = other DNA; note = crPTPN6_4
gcacaggcaccatcattgtc

SEQ ID NO: 552 - DNA, 20 bp, synthetic construct
  source 1..20: mol_type = other DNA; note = crPTPN6_5
cgaccctccctttccagaac

SEQ ID NO: 553 - DNA, 20 bp, synthetic construct
  source 1..20: mol_type = other DNA; note = crPTPN6_6
gatggtgaggtaagggcctg

SEQ ID NO: 554 - DNA, 20 bp, synthetic construct
  source 1..20: mol_type = other DNA; note = crPTPN6_7
ggcccctctctgtgaatgtc

SEQ ID NO: 555 - DNA, 20 bp, synthetic construct
  source 1..20: mol_type = other DNA; note = crPTPN6_8
gtggcctgggtcttaccttc

SEQ ID NO: 556 - DNA, 20 bp, synthetic construct
  source 1..20: mol_type = other DNA; note = crPTPN6_9
gtggcctgggtcttaccttc

SEQ ID NO: 557 - DNA, 20 bp, synthetic construct
  source 1..20: mol_type = other DNA; note = crPTPN6_10
gtggcctgggtcttaccttc

SEQ ID NO: 558 - DNA, 20 bp, synthetic construct
  source 1..20: mol_type = other DNA; note = crPTPN6_11
gtggcctgggtcttaccttc

SEQ ID NO: 559 - DNA, 20 bp, synthetic construct
  source 1..20: mol_type = other DNA; note = crPTPN6_12
gtggcctgggtcttaccttc

SEQ ID NO: 560 - DNA, 20 bp, synthetic construct
  source 1..20: mol_type = other DNA; note = crPTPN6_13
gtggcctgggtcttaccttc

SEQ ID NO: 561 - DNA, 20 bp, synthetic construct
  source 1..20: mol_type = other DNA; note = crPTPN6_14
ctggacgtttcttgtgcgtg

SEQ ID NO: 562 - DNA, 20 bp, synthetic construct
  source 1..20: mol_type = other DNA; note = crPTPN6_15
ctggacgtttcttgtgcgtg

SEQ ID NO: 563 - DNA, 22 bp, synthetic construct
  source 1..22: mol_type = other DNA; note = crPTPN6_16
ggagggtctgcctgggcttgaa

SEQ ID NO: 564 - DNA, 20 bp, synthetic construct
  source 1..20: mol_type = other DNA; note = crPTPN6_17
gatggtgaggtaagggcctg

SEQ ID NO: 565 - DNA, 20 bp, synthetic construct
  source 1..20: mol_type = other DNA; note = crPTPN6_18
ctgaggctcctgtctgtgac

SEQ ID NO: 566 - DNA, 21 bp, synthetic construct
  source 1..21: mol_type = other DNA; note = crPTPN6_19
ctcaagtcctgtgaatggcct

SEQ ID NO: 567 - DNA, 21 bp, synthetic construct
  source 1..21: mol_type = other DNA; note = crPTPN6_20
ctcaagtcctgtgaatggcct

SEQ ID NO: 568 - DNA, 20 bp, synthetic construct
  source 1..20: mol_type = other DNA; note = crPTPN6_21
gacttctcgctcttccccac

SEQ ID NO: 569 - DNA, 20 bp, synthetic construct
  source 1..20: mol_type = other DNA; note = crPTPN6_22
gacttctcgctcttccccac

SEQ ID NO: 570 - DNA, 19 bp, synthetic construct
  source 1..19: mol_type = other DNA; note = crPTPN6_23
gacaccttcccctccttgc

SEQ ID NO: 571 - DNA, 20 bp, synthetic construct
  source 1..20: mol_type = other DNA; note = crPTPN6_24
ccgatggatgccctctttgg

SEQ ID NO: 572 - DNA, 20 bp, synthetic construct
  source 1..20: mol_type = other DNA; note = crPTPN6_25
gctgatgctcatttccccac

SEQ ID NO: 573 - DNA, 20 bp, synthetic construct
  source 1..20: mol_type = other DNA; note = crPTPN6_26
gatgctcatttccccaccca

SEQ ID NO: 574 - DNA, 22 bp, synthetic construct
  source 1..22: mol_type = other DNA; note = crPTPN6_27
ctctccgcccactcccagttga

SEQ ID NO: 575 - DNA, 22 bp, synthetic construct
  source 1..22: mol_type = other DNA; note = crPTPN6_28
cttgcatgggtgagggtggcag

SEQ ID NO: 576 - DNA, 22 bp, synthetic construct
  source 1..22: mol_type = other DNA; note = crPTPN6_29
gctcactgtcttggggtgcgtc

SEQ ID NO: 577 - DNA, 22 bp, synthetic construct
  source 1..22: mol_type = other DNA; note = crPTPN6_30
gctcactgtcttggggtgcgtc

SEQ ID NO: 578 - DNA, 22 bp, synthetic construct
  source 1..22: mol_type = other DNA; note = crPTPN6_31
gctcactgtcttggggtgcgtc

SEQ ID NO: 579 - DNA, 20 bp, synthetic construct
  source 1..20: mol_type = other DNA; note = crPTPN6_32
cccatccgtccatccaacaa

SEQ ID NO: 580 - DNA, 20 bp, synthetic construct
  source 1..20: mol_type = other DNA; note = crPTPN6_33
cccatccgtccatccaacaa

SEQ ID NO: 581 - DNA, 20 bp, synthetic construct
  source 1..20: mol_type = other DNA; note = crPTPN6_34
cgaccctccctttccagaac

SEQ ID NO: 582 - DNA, 20 bp, synthetic construct
  source 1..20: mol_type = other DNA; note = crPTPN6_35
ggccctactctgtgaccaac

SEQ ID NO: 583 - DNA, 20 bp, synthetic construct
  source 1..20: mol_type = other DNA; note = crPTPN6_36
cacggtagacaggaggcaag

SEQ ID NO: 584 - DNA, 20 bp, synthetic construct
  source 1..20: mol_type = other DNA; note = crPTPN6_37
gtcgggtagggtgagatgga

SEQ ID NO: 585 - DNA, 20 bp, synthetic construct
  source 1..20: mol_type = other DNA; note = crPTPN6_38
cctgattcgggagatctggc

SEQ ID NO: 586 - DNA, 20 bp, synthetic construct
  source 1..20: mol_type = other DNA; note = crPTPN6_39
cctgattcgggagatctggc

SEQ ID NO: 587 - DNA, 20 bp, synthetic construct
  source 1..20: mol_type = other DNA; note = crPTPN6_40
cctgattcgggagatctggc

SEQ ID NO: 588 - DNA, 20 bp, synthetic construct
  source 1..20: mol_type = other DNA; note = crPTPN6_41
gcttgactggcctctgatgg

SEQ ID NO: 589 - DNA, 20 bp, synthetic construct
  source 1..20: mol_type = other DNA; note = crPTPN6_42
ggcctggactgtgacattga

SEQ ID NO: 590 - DNA, 20 bp, synthetic construct
  source 1..20: mol_type = other DNA; note = crPTPN6_43
ggcctggactgtgacattga

SEQ ID NO: 591 - DNA, 20 bp, synthetic construct
  source 1..20: mol_type = other DNA; note = crPTPN6_44
ggcctggactgtgacattga

SEQ ID NO: 592 - DNA, 21 bp, synthetic construct
  source 1..21: mol_type = other DNA; note = crPTPN6_45
ctctactcctgcaccgactgg

SEQ ID NO: 593 - DNA, 20 bp, synthetic construct
  source 1..20: mol_type = other DNA; note = crPTPN6_46
ttcaggcttggttctcaccc

SEQ ID NO: 594 - DNA, 20 bp, synthetic construct
  source 1..20: mol_type = other DNA; note = crPTPN6_47
gcctctgtcctctaggagct

SEQ ID NO: 595 - DNA, 20 bp, synthetic construct
  source 1..20: mol_type = other DNA; note = crPTPN6_48
gcctctgtcctctaggagct

SEQ ID NO: 596 - DNA, 20 bp, synthetic construct
  source 1..20: mol_type = other DNA; note = crPTPN6_49
ctgtgctgtctcctgacctg

SEQ ID NO: 597 - DNA, 20 bp, synthetic construct
  source 1..20: mol_type = other DNA; note = crPTPN6_50
ctgtgctgtctcctgacctg

SEQ ID NO: 598 - DNA, 20 bp, synthetic construct
  source 1..20: mol_type = other DNA; note = crPTPN6_51
ctgtgctgtctcctgacctg

SEQ ID NO: 599 - DNA, 20 bp, synthetic construct
  source 1..20: mol_type = other DNA; note = crPTPN6_52
atggaggggagaagtttgcg

SEQ ID NO: 600 - DNA, 20 bp, synthetic construct
  source 1..20: mol_type = other DNA; note = crPTPN6_53
ggcccctctctgtgaatgtc

SEQ ID NO: 601 - DNA, 20 bp, synthetic construct
  source 1..20: mol_type = other DNA; note = crTIGIT_1
aagaggccacatctgcttcc

SEQ ID NO: 602 - DNA, 20 bp, synthetic construct
  source 1..20: mol_type = other DNA; note = crTIGIT_2
ggctccagtcccatggttac

SEQ ID NO: 603 - DNA, 20 bp, synthetic construct
  source 1..20: mol_type = other DNA; note = crTIGIT_3
atgtcacctctcctccacca

SEQ ID NO: 604 - DNA, 20 bp, synthetic construct
  source 1..20: mol_type = other DNA; note = crTIGIT_4
cccaggactcacatgtgctt

SEQ ID NO: 605 - DNA, 20 bp, synthetic construct
  source 1..20: mol_type = other DNA; note = crTIGIT_5
atgtcacctctcctccacca

SEQ ID NO: 606 - DNA, 20 bp, synthetic construct
  source 1..20: mol_type = other DNA; note = crTIGIT_6
aagaggccacatctgcttcc

SEQ ID NO: 607 - DNA, 20 bp, synthetic construct
  source 1..20: mol_type = other DNA; note = crTIGIT_7
atgtcacctctcctccacca

SEQ ID NO: 608 - DNA, 20 bp, synthetic construct
  source 1..20: mol_type = other DNA; note = crTIGIT_8
atgtcacctctcctccacca

SEQ ID NO: 609 - DNA, 20 bp, synthetic construct
  source 1..20: mol_type = other DNA; note = crTIGIT_9
ggcacatctccccatccttc

SEQ ID NO: 610 - DNA, 20 bp, synthetic construct
  source 1..20: mol_type = other DNA; note = crTIGIT_10
ggcacatctccccatccttc

SEQ ID NO: 611 - DNA, 20 bp, synthetic construct
  source 1..20: mol_type = other DNA; note = crTIGIT_11
ggcacatctccccatccttc

SEQ ID NO: 612 - DNA, 20 bp, synthetic construct
  source 1..20: mol_type = other DNA; note = crTIGIT_12
ggcacatctccccatccttc

SEQ ID NO: 613 - DNA, 20 bp, synthetic construct
  source 1..20: mol_type = other DNA; note = crTIGIT_13
ggttacacaaagggcttggc

SEQ ID NO: 614 - DNA, 20 bp, synthetic construct
  source 1..20: mol_type = other DNA; note = crTIGIT_14
gtcctccctctagtggctga

SEQ ID NO: 615 - DNA, 20 bp, synthetic construct
  source 1..20: mol_type = other DNA; note = crTIGIT_15
gtcctccctctagtggctga

SEQ ID NO: 616 - DNA, 20 bp, synthetic construct
  source 1..20: mol_type = other DNA; note = crTIGIT_16
agctgtaacgcggttgagaa

SEQ ID NO: 617 - DNA, 20 bp, synthetic construct
  source 1..20: mol_type = other DNA; note = crTIGIT_17
agctgtaacgcggttgagaa

SEQ ID NO: 618 - DNA, 23 bp, synthetic construct
  source 1..23: mol_type = other DNA; note = crTIGIT_18
agtttgctggtgtgcatgtgtgt

SEQ ID NO: 619 - DNA, 23 bp, synthetic construct
  source 1..23: mol_type = other DNA; note = crTIGIT_19
agtttgctggtgtgcatgtgtgt

SEQ ID NO: 620 - DNA, 23 bp, synthetic construct
  source 1..23: mol_type = other DNA; note = crTIGIT_20
agtttgctggtgtgcatgtgtgt

SEQ ID NO: 621 - DNA, 23 bp, synthetic construct
  source 1..23: mol_type = other DNA; note = crTIGIT_21
agtttgctggtgtgcatgtgtgt

SEQ ID NO: 622 - DNA, 22 bp, synthetic construct
  source 1..22: mol_type = other DNA; note = crTIGIT_22
agaagaaagccctcagaatcca

SEQ ID NO: 623 - DNA, 20 bp, synthetic construct
  source 1..20: mol_type = other DNA; note = crTIGIT_23
tgtggaaggtgacctcagga

SEQ ID NO: 624 - DNA, 20 bp, synthetic construct
  source 1..20: mol_type = other DNA; note = crTIGIT_24
ggaggagcaacaggatggac

SEQ ID NO: 625 - DNA, 20 bp, synthetic construct
  source 1..20: mol_type = other DNA; note = crTIGIT_25
gaagctgtgtccaggcagaa

SEQ ID NO: 626 - DNA, 20 bp, synthetic construct
  source 1..20: mol_type = other DNA; note = crTIGIT_26
gaagctgtgtccaggcagaa

SEQ ID NO: 627 - DNA, 20 bp, synthetic construct
  source 1..20: mol_type = other DNA; note = crTIGIT_27
ggaggagcaacaggatggac

SEQ ID NO: 628 - DNA, 20 bp, synthetic construct
  source 1..20: mol_type = other DNA; note = crTIGIT_28
cccaggactcacatgtgctt

SEQ ID NO: 629 - DNA, 20 bp, synthetic construct
  source 1..20: mol_type = other DNA; note = crTIGIT_29
cccaggactcacatgtgctt

SEQ ID NO: 630 - DNA, 20 bp, synthetic construct
  source 1..20: mol_type = other DNA; note = crTIGIT_30
cccaggactcacatgtgctt

SEQ ID NO: 631 - DNA, 20 bp, synthetic construct
  source 1..20: mol_type = other DNA; note = crTIGIT_31
atgtcacctctcctccacca

SEQ ID NO: 632 - DNA, 21 bp, synthetic construct
  source 1..21: mol_type = other DNA; note = crTIM3_1
ggccatccttgtatctctccc

SEQ ID NO: 633 - DNA, 20 bp, synthetic construct
  source 1..20: mol_type = other DNA; note = crTIM3_2
gcacggagatatccatgcct

SEQ ID NO: 634 - DNA, 21 bp, synthetic construct
  source 1..21: mol_type = other DNA; note = crTIM3_3
ggccatccttgtatctctccc

SEQ ID NO: 635 - DNA, 20 bp, synthetic construct
  source 1..20: mol_type = other DNA; note = crTIM3_4
tgtctccaccacttccctct

SEQ ID NO: 636 - DNA, 20 bp, synthetic construct
  source 1..20: mol_type = other DNA; note = crTIM3_5
gatccggcagcagtagatcc

SEQ ID NO: 637 - DNA, 20 bp, synthetic construct
  source 1..20: mol_type = other DNA; note = crTIM3_6
cccttgtcctctgtacagca

SEQ ID NO: 638 - DNA, 20 bp, synthetic construct
  source 1..20: mol_type = other DNA; note = crTIM3_7
tctcctttgcggaaatcccc

SEQ ID NO: 639 - DNA, 20 bp, synthetic construct
  source 1..20: mol_type = other DNA; note = crTIM3_8
gatccggcagcagtagatcc

SEQ ID NO: 640 - DNA, 20 bp, synthetic construct
  source 1..20: mol_type = other DNA; note = crTIM3_9
gatccggcagcagtagatcc

SEQ ID NO: 641 - DNA, 20 bp, synthetic construct
  source 1..20: mol_type = other DNA; note = crTIM3_10
gatccggcagcagtagatcc

SEQ ID NO: 642 - DNA, 20 bp, synthetic construct
  source 1..20: mol_type = other DNA; note = crTIM3_11
gatccggcagcagtagatcc

SEQ ID NO: 643 - DNA, 20 bp, synthetic construct
  source 1..20: mol_type = other DNA; note = crTIM3_12
gatccggcagcagtagatcc

SEQ ID NO: 644 - DNA, 20 bp, synthetic construct
  source 1..20: mol_type = other DNA; note = crTIM3_13
gcaaatgtccactcacctgg

SEQ ID NO: 645 - DNA, 20 bp, synthetic construct
  source 1..20: mol_type = other DNA; note = crTIM3_14
gcaaatgtccactcacctgg

SEQ ID NO: 646 - DNA, 20 bp, synthetic construct
  source 1..20: mol_type = other DNA; note = crTIM3_15
tcttagtggccctcctccag

SEQ ID NO: 647 - DNA, 20 bp, synthetic construct
  source 1..20: mol_type = other DNA; note = crTIM3_16
cccttgtcctctgtacagca

SEQ ID NO: 648 - DNA, 20 bp, synthetic construct
  source 1..20: mol_type = other DNA; note = crTIM3_17
tcttagtggccctcctccag

SEQ ID NO: 649 - DNA, 20 bp, synthetic construct
  source 1..20: mol_type = other DNA; note = crTIM3_18
tcttagtggccctcctccag

SEQ ID NO: 650 - DNA, 20 bp, synthetic construct
  source 1..20: mol_type = other DNA; note = crTIM3_19
actgagcatcaccaatgggg

SEQ ID NO: 651 - DNA, 20 bp, synthetic construct
  source 1..20: mol_type = other DNA; note = crTIM3_20
gtcccctggtggtaagcatc

SEQ ID NO: 652 - DNA, 20 bp, synthetic construct
  source 1..20: mol_type = other DNA; note = crTIM3_21
gtcccctggtggtaagcatc

SEQ ID NO: 653 - DNA, 20 bp, synthetic construct
  source 1..20: mol_type = other DNA; note = crTIM3_22
aaagattccctcctctgccc

SEQ ID NO: 654 - DNA, 21 bp, synthetic construct
  source 1..21: mol_type = other DNA; note = crTIM3_23
gccagctaaagattccctcct

SEQ ID NO: 655 - DNA, 20 bp, synthetic construct
  source 1..20: mol_type = other DNA; note = crTIM3_24
gcacggagatatccatgcct

SEQ ID NO: 656 - DNA, 20 bp, synthetic construct
  source 1..20: mol_type = other DNA; note = crTIM3_25
cccttgtcctctgtacagca

SEQ ID NO: 657 - DNA, 22 bp, synthetic construct
  source 1..22: mol_type = other DNA; note = crTIM3_26
tgagtacaacatagctcacaaa

SEQ ID NO: 658 - DNA, 22 bp, synthetic construct
  source 1..22: mol_type = other DNA; note = crTIM3_27
tgagtacaacatagctcacaaa

SEQ ID NO: 659 - DNA, 22 bp, synthetic construct
  source 1..22: mol_type = other DNA; note = crTIM3_28
caaggacaaggtgggcatgaag

SEQ ID NO: 660 - DNA, 20 bp, synthetic construct
  source 1..20: mol_type = other DNA; note = crTIM3_29
cacagatccctgctccgatg

SEQ ID NO: 661 - DNA, 20 bp, synthetic construct
  source 1..20: mol_type = other DNA; note = crTIM3_30
cacagatccctgctccgatg

SEQ ID NO: 662 - DNA, 20 bp, synthetic construct
  source 1..20: mol_type = other DNA; note = crTIM3_31
cgccgaagataagagccaga

SEQ ID NO: 663 - DNA, 22 bp, synthetic construct
  source 1..22: mol_type = other DNA; note = crTIM3_32
ggatttggatggacaaaagggt

SEQ ID NO: 664 - DNA, 22 bp, synthetic construct
  source 1..22: mol_type = other DNA; note = crTIM3_33
ggatttggatggacaaaagggt

SEQ ID NO: 665 - DNA, 20 bp, synthetic construct
  source 1..20: mol_type = other DNA; note = crTIM3_34
caaagccccaggacaggatt

SEQ ID NO: 666 - DNA, 20 bp, synthetic construct
  source 1..20: mol_type = other DNA; note = crTIM3_35
caaagccccaggacaggatt

SEQ ID NO: 667 - DNA, 20 bp, synthetic construct
  source 1..20: mol_type = other DNA; note = crTIM3_36
cccttgtcctctgtacagca

SEQ ID NO: 668 - DNA, 20 bp, synthetic construct
  source 1..20: mol_type = other DNA; note = crTIM3_37
caaagccccaggacaggatt

SEQ ID NO: 669 - DNA, 20 bp, synthetic construct
  source 1..20: mol_type = other DNA; note = crTIM3_38
caaagccccaggacaggatt

SEQ ID NO: 670 - DNA, 20 bp, synthetic construct
  source 1..20: mol_type = other DNA; note = crTIM3_39
caaagccccaggacaggatt

SEQ ID NO: 671 - DNA, 20 bp, synthetic construct
  source 1..20: mol_type = other DNA; note = crTIM3_40
cattgggctcctccacttca

SEQ ID NO: 672 - DNA, 20 bp, synthetic construct
  source 1..20: mol_type = other DNA; note = crTIM3_41
cattgggctcctccacttca

SEQ ID NO: 673 - DNA, 20 bp, synthetic construct
  source 1..20: mol_type = other DNA; note = crTIM3_42
cattgggctcctccacttca

SEQ ID NO: 674 - DNA, 20 bp, synthetic construct
  source 1..20: mol_type = other DNA; note = crTIM3_43
cattgcaaagcgacaaccca

SEQ ID NO: 675 - DNA, 20 bp, synthetic construct
  source 1..20: mol_type = other DNA; note = crTIM3_44
cattgcaaagcgacaaccca

SEQ ID NO: 676 - DNA, 20 bp, synthetic construct
  source 1..20: mol_type = other DNA; note = crTIM3_45
cattgcaaagcgacaaccca

SEQ ID NO: 677 - DNA, 20 bp, synthetic construct
  source 1..20: mol_type = other DNA; note = crTIM3_46
cattgcaaagcgacaaccca

SEQ ID NO: 678 - DNA, 20 bp, synthetic construct
  source 1..20: mol_type = other DNA; note = crTIM3_47
cagtgcaggtcccagttcaa

SEQ ID NO: 679 - DNA, 20 bp, synthetic construct
  source 1..20: mol_type = other DNA; note = crTIM3_48
cagtgcaggtcccagttcaa

SEQ ID NO: 680 - DNA, 20 bp, synthetic construct
  source 1..20: mol_type = other DNA; note = crTIM3_49
tcaaacacaggacaggctcc

SEQ ID NO: 681 - DNA, 20 bp, synthetic construct
  source 1..20: mol_type = other DNA; note = crTIM3_50
tctcctttgcggaaatcccc

SEQ ID NO: 682 - DNA, 20 bp, synthetic construct
  source 1..20: mol_type = other DNA; note = crTIM3_51
tctcctttgcggaaatcccc

SEQ ID NO: 683 - DNA, 20 bp, synthetic construct
  source 1..20: mol_type = other DNA; note = crAAVS1
catctctcctccctcaccca

SEQ ID NO: 684 - DNA, 20 bp, synthetic construct
  source 1..20: mol_type = other DNA; note = crCD247_1
ctagaagttccctgccgtcg

SEQ ID NO: 685 - DNA, 20 bp, synthetic construct
  source 1..20: mol_type = other DNA; note = crCD247_2
ctagaagttccctgccgtcg

SEQ ID NO: 686 - DNA, 20 bp, synthetic construct
  source 1..20: mol_type = other DNA; note = crCD247_3
gcccctctgaacatccatca

SEQ ID NO: 687 - DNA, 20 bp, synthetic construct
  source 1..20: mol_type = other DNA; note = crCD247_4
gcccttcctccaactttcca

SEQ ID NO: 688 - DNA, 20 bp, synthetic construct
  source 1..20: mol_type = other DNA; note = crCD247_5
ggcgaggctgacttacgtta

SEQ ID NO: 689 - DNA, 20 bp, synthetic construct
  source 1..20: mol_type = other DNA; note = crCD247_6
ggtagcacagggaggagaga

SEQ ID NO: 690 - DNA, 20 bp, synthetic construct
  source 1..20: mol_type = other DNA; note = crCD247_7
gcccttcctccaactttcca

SEQ ID NO: 691 - DNA, 20 bp, synthetic construct
  source 1..20: mol_type = other DNA; note = crCD247_8
ctggttgtgggcagagaagt

SEQ ID NO: 692 - DNA, 20 bp, synthetic construct
  source 1..20: mol_type = other DNA; note = crCD247_9
cgtgtctggaggaccaagag

SEQ ID NO: 693 - DNA, 20 bp, synthetic construct
  source 1..20: mol_type = other DNA; note = crCD247_10
tggagccttgattgtgggag

SEQ ID NO: 694 - DNA, 20 bp, synthetic construct
  source 1..20: mol_type = other DNA; note = crCD247_11
tggagccttgattgtgggag

SEQ ID NO: 695 - DNA, 20 bp, synthetic construct
  source 1..20: mol_type = other DNA; note = crCD247_12
atcttgccccttgtcaggtg

SEQ ID NO: 696 - DNA, 20 bp, synthetic construct
  source 1..20: mol_type = other DNA; note = crCD247_13
atcttgccccttgtcaggtg

SEQ ID NO: 697 - DNA, 20 bp, synthetic construct
  source 1..20: mol_type = other DNA; note = crCD247_14
atcttgccccttgtcaggtg

SEQ ID NO: 698 - DNA, 20 bp, synthetic construct
  source 1..20: mol_type = other DNA; note = crCD247_15
ttagttgccaaggagcggag

SEQ ID NO: 699 - DNA, 20 bp, synthetic construct
  source 1..20: mol_type = other DNA; note = crCD247_16
gtctggcctttgagtggtga

SEQ ID NO: 700 - DNA, 20 bp, synthetic construct
  source 1..20: mol_type = other DNA; note = crCD247_17
gtctggcctttgagtggtga

SEQ ID NO: 701 - DNA, 20 bp, synthetic construct
  source 1..20: mol_type = other DNA; note = crCD247_18
gtctggcctttgagtggtga

SEQ ID NO: 702 - DNA, 20 bp, synthetic construct
  source 1..20: mol_type = other DNA; note = crCD247_19
ataatctgggcgtctgcagg

SEQ ID NO: 703 - DNA, 20 bp, synthetic construct
  source 1..20: mol_type = other DNA; note = crCD247_20
tgtgttgcagttcagcagga

SEQ ID NO: 704 - DNA, 20 bp, synthetic construct
  source 1..20: mol_type = other DNA; note = crCD247_21
tggttgcagagtgagctgag

SEQ ID NO: 705 - DNA, 20 bp, synthetic construct
  source 1..20: mol_type = other DNA; note = crCD247_22
tggttgcagagtgagctgag

SEQ ID NO: 706 - DNA, 20 bp, synthetic construct
  source 1..20: mol_type = other DNA; note = crCD247_23
gcccctgcccctctttttat

SEQ ID NO: 707 - DNA, 20 bp, synthetic construct
  source 1..20: mol_type = other DNA; note = crCD247_24
gcccctgcccctctttttat

SEQ ID NO: 708 - DNA, 20 bp, synthetic construct
  source 1..20: mol_type = other DNA; note = crCD247_25
tggttgcagagtgagctgag

SEQ ID NO: 709 - DNA, 20 bp, synthetic construct
  source 1..20: mol_type = other DNA; note = crCD247_26
gcccttcctccaactttcca

SEQ ID NO: 710 - DNA, 20 bp, synthetic construct
  source 1..20: mol_type = other DNA; note = crCTLA4_1
ggccatgaaggagcatgagt

SEQ ID NO: 711 - DNA, 20 bp, synthetic construct
  source 1..20: mol_type = other DNA; note = crCTLA4_2
tgaagacctgaacaccgctc

SEQ ID NO: 712 - DNA, 20 bp, synthetic construct
  source 1..20: mol_type = other DNA; note = crCTLA4_3
aggtgactgaagtctgtgcg

SEQ ID NO: 713 - DNA, 20 bp, synthetic construct
  source 1..20: mol_type = other DNA; note = crCTLA4_4
atcatgtaggttgccgcaca

SEQ ID NO: 714 - DNA, 21 bp, synthetic construct
  source 1..21: mol_type = other DNA; note = crCTLA4_5
cctcctccatcttcatgctcc

SEQ ID NO: 715 - DNA, 21 bp, synthetic construct
  source 1..21: mol_type = other DNA; note = crCTLA4_6
agtccttgattctgtgtgggt

SEQ ID NO: 716 - DNA, 20 bp, synthetic construct
  source 1..20: mol_type = other DNA; note = crCTLA4_7
atcatgtaggttgccgcaca

SEQ ID NO: 717 - DNA, 20 bp, synthetic construct
  source 1..20: mol_type = other DNA; note = crCTLA4_8
atcatgtaggttgccgcaca

SEQ ID NO: 718 - DNA, 20 bp, synthetic construct
  source 1..20: mol_type = other DNA; note = crCTLA4_9
atcatgtaggttgccgcaca

SEQ ID NO: 719 - DNA, 20 bp, synthetic construct
  source 1..20: mol_type = other DNA; note = crCTLA4_10
atcatgtaggttgccgcaca

SEQ ID NO: 720 - DNA, 20 bp, synthetic construct
  source 1..20: mol_type = other DNA; note = crCTLA4_11
tgatttccactggaggtgcc

SEQ ID NO: 721 - DNA, 20 bp, synthetic construct
  source 1..20: mol_type = other DNA; note = crCTLA4_12
tgatttccactggaggtgcc

SEQ ID NO: 722 - DNA, 20 bp, synthetic construct
  source 1..20: mol_type = other DNA; note = crCTLA4_13
gagctccaccttgcagatgt

SEQ ID NO: 723 - DNA, 21 bp, synthetic construct
  source 1..21: mol_type = other DNA; note = crCTLA4_14
cctcctccatcttcatgctcc

SEQ ID NO: 724 - DNA, 20 bp, synthetic construct
  source 1..20: mol_type = other DNA; note = crCTLA4_15
aaatctgggttccgttgcct

SEQ ID NO: 725 - DNA, 20 bp, synthetic construct
  source 1..20: mol_type = other DNA; note = crCTLA4_16
aaatctgggttccgttgcct

SEQ ID NO: 726 - DNA, 20 bp, synthetic construct
  source 1..20: mol_type = other DNA; note = crCTLA4_17
aaatctgggttccgttgcct

SEQ ID NO: 727 - DNA, 20 bp, synthetic construct
  source 1..20: mol_type = other DNA; note = crCTLA4_18
ggttgccacccacaataagc

SEQ ID NO: 728 - DNA, 20 bp, synthetic construct
  source 1..20: mol_type = other DNA; note = crCTLA4_19
ggttgccacccacaataagc

SEQ ID NO: 729 - DNA, 20 bp, synthetic construct
  source 1..20: mol_type = other DNA; note = crCTLA4_20
catcagcaccacactcacca

SEQ ID NO: 730 - DNA, 20 bp, synthetic construct
  source 1..20: mol_type = other DNA; note = crCTLA4_21
catcagcaccacactcacca

SEQ ID NO: 731 - DNA, 20 bp, synthetic construct
  source 1..20: mol_type = other DNA; note = crCTLA4_22
atccccatcagacatggtgc

SEQ ID NO: 732 - DNA, 22 bp, synthetic construct
  source 1..22: mol_type = other DNA; note = crCTLA4_23
gcaccacactcaccattttgct

SEQ ID NO: 733 - DNA, 20 bp, synthetic construct
  source 1..20: mol_type = other DNA; note = crCTLA4_24
atccccatcagacatggtgc

SEQ ID NO: 734 - DNA, 21 bp, synthetic construct
  source 1..21: mol_type = other DNA; note = crCTLA4_25
cctcctccatcttcatgctcc

SEQ ID NO: 735 - DNA, 20 bp, synthetic construct
  source 1..20: mol_type = other DNA; note = crCTLA4_26
atccccatcagacatggtgc

SEQ ID NO: 736 - DNA, 20 bp, synthetic construct
  source 1..20: mol_type = other DNA; note = crCTLA4_27
atccccatcagacatggtgc

SEQ ID NO: 737 - DNA, 20 bp, synthetic construct
  source 1..20: mol_type = other DNA; note = crCTLA4_28
atccccatcagacatggtgc

SEQ ID NO: 738 - DNA, 20 bp, synthetic construct
  source 1..20: mol_type = other DNA; note = crCTLA4_29
atccccatcagacatggtgc

SEQ ID NO: 739 - DNA, 21 bp, synthetic construct
  source 1..21: mol_type = other DNA; note = crCTLA4_30
tgcctcagctcttggaaattg

SEQ ID NO: 740 - DNA, 21 bp, synthetic construct
  source 1..21: mol_type = other DNA; note = crCTLA4_31
tgcctcagctcttggaaattg

SEQ ID NO: 741 - DNA, 21 bp, synthetic construct
  source 1..21: mol_type = other DNA; note = crCTLA4_32
tgcctcagctcttggaaattg

SEQ ID NO: 742 - DNA, 21 bp, synthetic construct
  source 1..21: mol_type = other DNA; note = crCTLA4_33
agaattgcctcagctcttgga

SEQ ID NO: 743 - DNA, 21 bp, synthetic construct
  source 1..21: mol_type = other DNA; note = crCTLA4_34
agaattgcctcagctcttgga

SEQ ID NO: 744 - DNA, 21 bp, synthetic construct
  source 1..21: mol_type = other DNA; note = crCTLA4_35
agaattgcctcagctcttgga

SEQ ID NO: 745 - DNA, 20 bp, synthetic construct
  source 1..20: mol_type = other DNA; note = crCTLA4_36
tgggttccgcatccaacttt

SEQ ID NO: 746 - DNA, 20 bp, synthetic construct
  source 1..20: mol_type = other DNA; note = crCTLA4_37
tgggttccgcatccaacttt

SEQ ID NO: 747 - DNA, 20 bp, synthetic construct
  source 1..20: mol_type = other DNA; note = crCTLA4_38
tcactgcctttgactgctga

SEQ ID NO: 748 - DNA, 20 bp, synthetic construct
  source 1..20: mol_type = other DNA; note = crCTLA4_39
tcactgcctttgactgctga

SEQ ID NO: 749 - DNA, 20 bp, synthetic construct
  source 1..20: mol_type = other DNA; note = crCTLA4_40
atcatgtaggttgccgcaca

SEQ ID NO: 750 - DNA, 20 bp, synthetic construct
  source 1..20: mol_type = other DNA; note = crCTLA4_41
atcatgtaggttgccgcaca

SEQ ID NO: 751 - DNA, 19 bp, synthetic construct
  source 1..19: mol_type = other DNA; note = crLAG3_1
agggagtgacacctcaggg

SEQ ID NO: 752 - DNA, 20 bp, synthetic construct
  source 1..20: mol_type = other DNA; note = crLAG3_2
gtgtccagagagctccacac

SEQ ID NO: 753 - DNA, 20 bp, synthetic construct
  source 1..20: mol_type = other DNA; note = crLAG3_3
tttgggtcctggcattctgg

SEQ ID NO: 754 - DNA, 22 bp, synthetic construct
  source 1..22: mol_type = other DNA; note = crLAG3_4
tggcgtttgggtcctggcattc

SEQ ID NO: 755 - DNA, 20 bp, synthetic construct
  source 1..20: mol_type = other DNA; note = crLAG3_5
ccagccaaggtcctgagaaa

SEQ ID NO: 756 - DNA, 22 bp, synthetic construct
  source 1..22: mol_type = other DNA; note = crLAG3_6
ccagagaggctttcggggtgga

SEQ ID NO: 757 - DNA, 20 bp, synthetic construct
  source 1..20: mol_type = other DNA; note = crLAG3_7
ttccggaaccaatgcacaga

SEQ ID NO: 758 - DNA, 20 bp, synthetic construct
  source 1..20: mol_type = other DNA; note = crLAG3_8
cttggggcaggaagaggaag

SEQ ID NO: 759 - DNA, 20 bp, synthetic construct
  source 1..20: mol_type = other DNA; note = crLAG3_9
cttggggcaggaagaggaag

SEQ ID NO: 760 - DNA, 20 bp, synthetic construct
  source 1..20: mol_type = other DNA; note = crLAG3_10
ctgtaggtgaggatgcagcc

SEQ ID NO: 761 - DNA, 20 bp, synthetic construct
  source 1..20: mol_type = other DNA; note = crLAG3_11
ctgtaggtgaggatgcagcc

SEQ ID NO: 762 - DNA, 22 bp, synthetic construct
  source 1..22: mol_type = other DNA; note = crLAG3_12
gggggcaggaaggagttgtggt

SEQ ID NO: 763 - DNA, 22 bp, synthetic construct
  source 1..22: mol_type = other DNA; note = crLAG3_13
gggggcaggaaggagttgtggt

SEQ ID NO: 764 - DNA, 22 bp, synthetic construct
  source 1..22: mol_type = other DNA; note = crLAG3_14
gggggcaggaaggagttgtggt

SEQ ID NO: 765 - DNA, 20 bp, synthetic construct
  source 1..20: mol_type = other DNA; note = crLAG3_15
acccacagcaatgacgtagg

SEQ ID NO: 766 - DNA, 20 bp, synthetic construct
  source 1..20: mol_type = other DNA; note = crLAG3_16
cagtgaggaaagaccgggtc

SEQ ID NO: 767 - DNA, 22 bp, synthetic construct
  source 1..22: mol_type = other DNA; note = crLAG3_17
ccagagaggctttcggggtgga

SEQ ID NO: 768 - DNA, 20 bp, synthetic construct
  source 1..20: mol_type = other DNA; note = crLAG3_18
cagtgaggaaagaccgggtc

SEQ ID NO: 769 - DNA, 20 bp, synthetic construct
  source 1..20: mol_type = other DNA; note = crLAG3_19
cagtgaggaaagaccgggtc

SEQ ID NO: 770 - DNA, 22 bp, synthetic construct
  source 1..22: mol_type = other DNA; note = crLAG3_20
ctgggcctggctcacatcctct

SEQ ID NO: 771 - DNA, 22 bp, synthetic construct
  source 1..22: mol_type = other DNA; note = crLAG3_21
ctgggcctggctcacatcctct

SEQ ID NO: 772 - DNA, 20 bp, synthetic construct
  source 1..20: mol_type = other DNA; note = crLAG3_22
gagggcagctactcctttcc

SEQ ID NO: 773 - DNA, 20 bp, synthetic construct
  source 1..20: mol_type = other DNA; note = crLAG3_23
gagggcagctactcctttcc

SEQ ID NO: 774 - DNA, 20 bp, synthetic construct
  source 1..20: mol_type = other DNA; note = crLAG3_24
gagggcagctactcctttcc

SEQ ID NO: 775 - DNA, 20 bp, synthetic construct
  source 1..20: mol_type = other DNA; note = crLAG3_25
ctctggaacttgtgcccagt

SEQ ID NO: 776 - DNA, 20 bp, synthetic construct
  source 1..20: mol_type = other DNA; note = crLAG3_26
ctctggaacttgtgcccagt

SEQ ID NO: 777 - DNA, 20 bp, synthetic construct
  source 1..20: mol_type = other DNA; note = crLAG3_27
gtgtccagagagctccacac

SEQ ID NO: 778 - DNA, 22 bp, synthetic construct
  source 1..22: mol_type = other DNA; note = crLAG3_28
ccagagaggctttcggggtgga

SEQ ID NO: 779 - DNA, 20 bp, synthetic construct
  source 1..20: mol_type = other DNA; note = crLAG3_29
gtgtccagagagctccacac

SEQ ID NO: 780 - DNA, 20 bp, synthetic construct
  source 1..20: mol_type = other DNA; note = crLAG3_30
gtgtccagagagctccacac

SEQ ID NO: 781 - DNA, 20 bp, synthetic construct
  source 1..20: mol_type = other DNA; note = crLAG3_31
gtgtccagagagctccacac

SEQ ID NO: 782 - DNA, 20 bp, synthetic construct
  source 1..20: mol_type = other DNA; note = crLAG3_32
ccagccaaggtcctgagaaa

SEQ ID NO: 783 - DNA, 20 bp, synthetic construct
  source 1..20: mol_type = other DNA; note = crLAG3_33
ctgctccaagaagcctctcc

SEQ ID NO: 784 - DNA, 20 bp, synthetic construct
  source 1..20: mol_type = other DNA; note = crLAG3_34
ctgctccaagaagcctctcc

SEQ ID NO: 785 - DNA, 20 bp, synthetic construct
  source 1..20: mol_type = other DNA; note = crLAG3_35
tttgggtcctggcattctgg

SEQ ID NO: 786 - DNA, 20 bp, synthetic construct
  source 1..20: mol_type = other DNA; note = crLAG3_36
gcagaaggctgagatcctgg

SEQ ID NO: 787 - DNA, 20 bp, synthetic construct
  source 1..20: mol_type = other DNA; note = crLAG3_37
tttgggtcctggcattctgg

SEQ ID NO: 788 - DNA, 22 bp, synthetic construct
  source 1..22: mol_type = other DNA; note = crLAG3_38
tggcgtttgggtcctggcattc

SEQ ID NO: 789 - DNA, 20 bp, synthetic construct
  source 1..20: mol_type = other DNA; note = crLAG3_39
aagccagagatcaggtccct

SEQ ID NO: 790 - DNA, 20 bp, synthetic construct
  source 1..20: mol_type = other DNA; note = crLAG3_40
aagccagagatcaggtccct

SEQ ID NO: 791 - DNA, 20 bp, synthetic construct
  source 1..20: mol_type = other DNA; note = crLAG3_41
ggctccagtcaccaaaagga

SEQ ID NO: 792 - DNA, 20 bp, synthetic construct
  source 1..20: mol_type = other DNA; note = crLAG3_42
ggctccagtcaccaaaagga

SEQ ID NO: 793 - DNA, 22 bp, synthetic construct
  source 1..22: mol_type = other DNA; note = crLAG3_43
tgagggcaagttcagggtccca

SEQ ID NO: 794 - DNA, 22 bp, synthetic construct
  source 1..22: mol_type = other DNA; note = crLAG3_44
tgagggcaagttcagggtccca

SEQ ID NO: 795 - DNA, 22 bp, synthetic construct
  source 1..22: mol_type = other DNA; note = crLAG3_45
tgagggcaagttcagggtccca

SEQ ID NO: 796 - DNA, 20 bp, synthetic construct
  source 1..20: mol_type = other DNA; note = crLAG3_46
ggttgagtgctggattcgga

SEQ ID NO: 797 - DNA, 22 bp, synthetic construct
  source 1..22: mol_type = other DNA; note = crLAG3_47
tgagggcaagttcagggtccca

SEQ ID NO: 798 - DNA, 22 bp, synthetic construct
  source 1..22: mol_type = other DNA; note = crLAG3_48
tgagggcaagttcagggtccca

SEQ ID NO: 799 - DNA, 22 bp, synthetic construct
  source 1..22: mol_type = other DNA; note = crLAG3_49
tgagggcaagttcagggtccca

SEQ ID NO: 800 - DNA, 22 bp, synthetic construct
  source 1..22: mol_type = other DNA; note = crLAG3_50
tgagggcaagttcagggtccca

SEQ ID NO: 801 - DNA, 20 bp, synthetic construct
  source 1..20: mol_type = other DNA; note = crLAG3_51
gactgggctgctgagatctg

SEQ ID NO: 802 - DNA, 20 bp, synthetic construct
  source 1..20: mol_type = other DNA; note = crLAG3_52
gactgggctgctgagatctg

SEQ ID NO: 803 - DNA, 20 bp, synthetic construct
  source 1..20: mol_type = other DNA; note = crLAG3_53
gactgggctgctgagatctg

SEQ ID NO: 804 - DNA, 18 bp, synthetic construct
  source 1..18: mol_type = other DNA; note = crLAG3_54
cacgctcagcaccgtgta

SEQ ID NO: 805 - DNA, 19 bp, synthetic construct
  source 1..19: mol_type = other DNA; note = crLAG3_55
cacatactcgaggcctggc

SEQ ID NO: 806 - DNA, 20 bp, synthetic construct
  source 1..20: mol_type = other DNA; note = crLAG3_56
ttccggaaccaatgcacaga

SEQ ID NO: 807 - DNA, 20 bp, synthetic construct
  source 1..20: mol_type = other DNA; note = crPDCD1_1
agcttgtccgtctggttgct

SEQ ID NO: 808 - DNA, 22 bp, synthetic construct
  source 1..22: mol_type = other DNA; note = crPDCD1_2
aggaaggaaggcacagtggatc

SEQ ID NO: 809 - DNA, 22 bp, synthetic construct
  source 1..22: mol_type = other DNA; note = crPDCD1_3
cgctaggaaagacaatggtggc

SEQ ID NO: 810 - DNA, 22 bp, synthetic construct
  source 1..22: mol_type = other DNA; note = crPDCD1_4
ccaagagcagtgtccatcctca

SEQ ID NO: 811 - DNA, 21 bp, synthetic construct
  source 1..21: mol_type = other DNA; note = crPDCD1_5
gaggtaggtgccgctgtcatt

SEQ ID NO: 812 - DNA, 22 bp, synthetic construct
  source 1..22: mol_type = other DNA; note = crPDCD1_6
tacctaagaaccatcctggccg

SEQ ID NO: 813 - DNA, 21 bp, synthetic construct
  source 1..21: mol_type = other DNA; note = crPDCD1_7
gaggtaggtgccgctgtcatt

SEQ ID NO: 814 - DNA, 21 bp, synthetic construct
  source 1..21: mol_type = other DNA; note = crPDCD1_8
gaggtaggtgccgctgtcatt

SEQ ID NO: 815 - DNA, 21 bp, synthetic construct
  source 1..21: mol_type = other DNA; note = crPDCD1_9
gaggtaggtgccgctgtcatt

SEQ ID NO: 816 - DNA, 21 bp, synthetic construct
  source 1..21: mol_type = other DNA; note = crPDCD1_10
gaggtaggtgccgctgtcatt

SEQ ID NO: 817 - DNA, 19 bp, synthetic construct
  source 1..19: mol_type = other DNA; note = crPDCD1_11
agctcctgatcctgtgcag

SEQ ID NO: 818 - DNA, 19 bp, synthetic construct
  source 1..19: mol_type = other DNA; note = crPDCD1_12
agctcctgatcctgtgcag

SEQ ID NO: 819 - DNA, 19 bp, synthetic construct
  source 1..19: mol_type = other DNA; note = crPDCD1_13
agctcctgatcctgtgcag

SEQ ID NO: 820 - DNA, 21 bp, synthetic construct
  source 1..21: mol_type = other DNA; note = crPDCD1_14
gacccagactagcagcaccag

SEQ ID NO: 821 - DNA, 21 bp, synthetic construct
  source 1..21: mol_type = other DNA; note = crPDCD1_15
gacccagactagcagcaccag

SEQ ID NO: 822 - DNA, 22 bp, synthetic construct
  source 1..22: mol_type = other DNA; note = crPDCD1_16
tacctaagaaccatcctggccg

SEQ ID NO: 823 - DNA, 21 bp, synthetic construct
  source 1..21: mol_type = other DNA; note = crPDCD1_17
gacccagactagcagcaccag

SEQ ID NO: 824 - DNA, 21 bp, synthetic construct
  source 1..21: mol_type = other DNA; note = crPDCD1_18
gacccagactagcagcaccag

SEQ ID NO: 825 - DNA, 21 bp, synthetic construct
  source 1..21: mol_type = other DNA; note = crPDCD1_19
gacccagactagcagcaccag

SEQ ID NO: 826 - DNA, 22 bp, synthetic construct
  source 1..22: mol_type = other DNA; note = crPDCD1_20
ggtcttctctcgccactggaaa

SEQ ID NO: 827 - DNA, 22 bp, synthetic construct
  source 1..22: mol_type = other DNA; note = crPDCD1_21
ggtcttctctcgccactggaaa

SEQ ID NO: 828 - DNA, 22 bp, synthetic construct
  source 1..22: mol_type = other DNA; note = crPDCD1_22
cgctaggaaagacaatggtggc

SEQ ID NO: 829 - DNA, 22 bp, synthetic construct
  source 1..22: mol_type = other DNA; note = crPDCD1_23
ccaagagcagtgtccatcctca

SEQ ID NO: 830 - DNA, 22 bp, synthetic construct
  source 1..22: mol_type = other DNA; note = crPDCD1_24
tacctaagaaccatcctggccg

SEQ ID NO: 831 - DNA, 22 bp, synthetic construct
  source 1..22: mol_type = other DNA; note = crPDCD1_25
ttctcctgaggaaatgcgctga

SEQ ID NO: 832 - DNA, 22 bp, synthetic construct
  source 1..22: mol_type = other DNA; note = crPDCD1_26
tacctaagaaccatcctggccg

SEQ ID NO: 833 - DNA, 20 bp, synthetic construct
  source 1..20: mol_type = other DNA; note = crPDCD1_27
agcttgtccgtctggttgct

SEQ ID NO: 834 - DNA, 20 bp, synthetic construct
  source 1..20: mol_type = other DNA; note = crPDCD1_28
agcttgtccgtctggttgct

SEQ ID NO: 835 - DNA, 20 bp, synthetic construct
  source 1..20: mol_type = other DNA; note = crPDCD1_29
agcttgtccgtctggttgct

SEQ ID NO: 836 - DNA, 20 bp, synthetic construct
  source 1..20: mol_type = other DNA; note = crPDCD1_30
agcttgtccgtctggttgct

SEQ ID NO: 837 - DNA, 22 bp, synthetic construct
  source 1..22: mol_type = other DNA; note = crPTPN1_1
ttcttgtacgagagagccagag

SEQ ID NO: 838 - DNA, 23 bp, synthetic construct
  source 1..23: mol_type = other DNA; note = crPTPN1_2
cacccaaatatcactggtgtgga

SEQ ID NO: 839 - DNA, 21 bp, synthetic construct
  source 1..21: mol_type = other DNA; note = crPTPN1_3
ggtaacatcttgccagaccca

SEQ ID NO: 840 - DNA, 23 bp, synthetic construct
  source 1..23: mol_type = other DNA; note = crPTPN11_4
gaaatacgacgttggtggaggag

SEQ ID NO: 841 - DNA, 23 bp, synthetic construct
  source 1..23: mol_type = other DNA; note = crPTPN11_5
tggtcagaaaacactgtgaaaag

SEQ ID NO: 842 - DNA, 22 bp, synthetic construct
  source 1..22: mol_type = other DNA; note = crPTPN11_6
gatcagccccttaacacgactc

SEQ ID NO: 843 - DNA, 23 bp, synthetic construct
  source 1..23: mol_type = other DNA; note = crPTPN11_7
gttgttgtggaaagtagtgctga

SEQ ID NO: 844 - DNA, 22 bp, synthetic construct
  source 1..22: mol_type = other DNA; note = crPTPN11_8
aggtacagaggtgctaggaatc

SEQ ID NO: 845 - DNA, 23 bp, synthetic construct
  source 1..23: mol_type = other DNA; note = crPTPN11_9
gaacaaaatctccagggtggctc

SEQ ID NO: 846 - DNA, 25 bp, synthetic construct
  source 1..25: mol_type = other DNA; note = crPTPN11_10
cccttggaggaatgtgtctactttt

SEQ ID NO: 847 - DNA, 22 bp, synthetic construct
  source 1..22: mol_type = other DNA; note = crPTPN6_1
gcgggtacttgaggtggatgat

SEQ ID NO: 848 - DNA, 20 bp, synthetic construct
  source 1..20: mol_type = other DNA; note = crPTPN6_2
ggagccctcacctctcacta

SEQ ID NO: 849 - DNA, 20 bp, synthetic construct
  source 1..20: mol_type = other DNA; note = crPTPN6_3
gagggtggagacctgtgaga

SEQ ID NO: 850 - DNA, 20 bp, synthetic construct
  source 1..20: mol_type = other DNA; note = crPTPN6_4
tgaacttgtactgcgcctcc

SEQ ID NO: 851 - DNA, 20 bp, synthetic construct
  source 1..20: mol_type = other DNA; note = crPTPN6_5
agaacaagtccagggaggga

SEQ ID NO: 852 - DNA, 20 bp, synthetic construct
  source 1..20: mol_type = other DNA; note = crPTPN6_6
tacctgacggagagcgagaa

SEQ ID NO: 853 - DNA, 20 bp, synthetic construct
  source 1..20: mol_type = other DNA; note = crPTPN6_7
actgagcacagaaagcacga

SEQ ID NO: 854 - DNA, 20 bp, synthetic construct
  source 1..20: mol_type = other DNA; note = crPTPN6_8
ctgccttacctcgcacatga

SEQ ID NO: 855 - DNA, 20 bp, synthetic construct
  source 1..20: mol_type = other DNA; note = crPTPN6_9
ctgccttacctcgcacatga

SEQ ID NO: 856 - DNA, 20 bp, synthetic construct
  source 1..20: mol_type = other DNA; note = crPTPN6_10
ctgccttacctcgcacatga

SEQ ID NO: 857 - DNA, 20 bp, synthetic construct
  source 1..20: mol_type = other DNA; note = crPTPN6_11
ctgccttacctcgcacatga

SEQ ID NO: 858 - DNA, 20 bp, synthetic construct
  source 1..20: mol_type = other DNA; note = crPTPN6_12
ctgccttacctcgcacatga

SEQ ID NO: 859 - DNA, 20 bp, synthetic construct
  source 1..20: mol_type = other DNA; note = crPTPN6_13
ctgccttacctcgcacatga

SEQ ID NO: 860 - DNA, 20 bp, synthetic construct
  source 1..20: mol_type = other DNA; note = crPTPN6_14
ggtccccagccttgaattca

SEQ ID NO: 861 - DNA, 20 bp, synthetic construct
  source 1..20: mol_type = other DNA; note = crPTPN6_15
ggtccccagccttgaattca

SEQ ID NO: 862 - DNA, 22 bp, synthetic construct
  source 1..22: mol_type = other DNA; note = crPTPN6_16
gtagacaaaggcgcctgaggcc

SEQ ID NO: 863 - DNA, 20 bp, synthetic construct
  source 1..20: mol_type = other DNA; note = crPTPN6_17
tacctgacggagagcgagaa

SEQ ID NO: 864 - DNA, 20 bp, synthetic construct
  source 1..20: mol_type = other DNA; note = crPTPN6_18
gtagacaaaggcgcctgagg

SEQ ID NO: 865 - DNA, 20 bp, synthetic construct
  source 1..20: mol_type = other DNA; note = crPTPN6_19
cagaagctcacatctggggg

SEQ ID NO: 866 - DNA, 20 bp, synthetic construct
  source 1..20: mol_type = other DNA; note = crPTPN6_20
cagaagctcacatctggggg

SEQ ID NO: 867 - DNA, 19 bp, synthetic construct
  source 1..19: mol_type = other DNA; note = crPTPN6_21
gcaaggaggggaaggtgtc

SEQ ID NO: 868 - DNA, 19 bp, synthetic construct
  source 1..19: mol_type = other DNA; note = crPTPN6_22
gcaaggaggggaaggtgtc

SEQ ID NO: 869 - DNA, 20 bp, synthetic construct
  source 1..20: mol_type = other DNA; note = crPTPN6_23
cggtatcctgggtgaatggg

SEQ ID NO: 870 - DNA, 20 bp, synthetic construct
  source 1..20: mol_type = other DNA; note = crPTPN6_24
gagggtggagacctgtgaga

SEQ ID NO: 871 - DNA, 20 bp, synthetic construct
  source 1..20: mol_type = other DNA; note = crPTPN6_25
gagggtggagacctgtgaga

SEQ ID NO: 872 - DNA, 20 bp, synthetic construct
  source 1..20: mol_type = other DNA; note = crPTPN6_26
gagggtggagacctgtgaga

SEQ ID NO: 873 - DNA, 22 bp, synthetic construct
  source 1..22: mol_type = other DNA; note = crPTPN6_27
cagcacaggccctgaaccactg

SEQ ID NO: 874 - DNA, 22 bp, synthetic construct
  source 1..22: mol_type = other DNA; note = crPTPN6_28
acccggcctttctccacctctc

SEQ ID NO: 875 - DNA, 22 bp, synthetic construct
  source 1..22: mol_type = other DNA; note = crPTPN6_29
tgccctggcatctgactgctct

SEQ ID NO: 876 - DNA, 22 bp, synthetic construct
  source 1..22: mol_type = other DNA; note = crPTPN6_30
tgccctggcatctgactgctct

SEQ ID NO: 877 - DNA, 22 bp, synthetic construct
  source 1..22: mol_type = other DNA; note = crPTPN6_31
tgccctggcatctgactgctct

SEQ ID NO: 878 - DNA, 20 bp, synthetic construct
  source 1..20: mol_type = other DNA; note = crPTPN6_32
ttcggttgtgtcatgctccc

SEQ ID NO: 879 - DNA, 20 bp, synthetic construct
  source 1..20: mol_type = other DNA; note = crPTPN6_33
ttcggttgtgtcatgctccc

SEQ ID NO: 880 - DNA, 20 bp, synthetic construct
  source 1..20: mol_type = other DNA; note = crPTPN6_34
agaacaagtccagggaggga

SEQ ID NO: 881 - DNA, 20 bp, synthetic construct
  source 1..20: mol_type = other DNA; note = crPTPN6_35
gccagatctcccgaatcagg

SEQ ID NO: 882 - DNA, 20 bp, synthetic construct
  source 1..20: mol_type = other DNA; note = crPTPN6_36
gcacaagagagtggccaaaa

SEQ ID NO: 883 - DNA, 20 bp, synthetic construct
  source 1..20: mol_type = other DNA; note = crPTPN6_37
atcatcctcacctgcagtgc

SEQ ID NO: 884 - DNA, 20 bp, synthetic construct
  source 1..20: mol_type = other DNA; note = crPTPN6_38
aacagctcatggcacttagc

SEQ ID NO: 885 - DNA, 20 bp, synthetic construct
  source 1..20: mol_type = other DNA; note = crPTPN6_39
aacagctcatggcacttagc

SEQ ID NO: 886 - DNA, 20 bp, synthetic construct
  source 1..20: mol_type = other DNA; note = crPTPN6_40
aacagctcatggcacttagc

SEQ ID NO: 887 - DNA, 20 bp, synthetic construct
  source 1..20: mol_type = other DNA; note = crPTPN6_41
tcaatgtcacagtccaggcc

SEQ ID NO: 888 - DNA, 20 bp, synthetic construct
  source 1..20: mol_type = other DNA; note = crPTPN6_42
agagggacagtgggaaggtg

SEQ ID NO: 889 - DNA, 20 bp, synthetic construct
  source 1..20: mol_type = other DNA; note = crPTPN6_43
agagggacagtgggaaggtg

SEQ ID NO: 890 - DNA, 20 bp, synthetic construct
  source 1..20: mol_type = other DNA; note = crPTPN6_44
agagggacagtgggaaggtg

SEQ ID NO: 891 - DNA, 22 bp, synthetic construct
  source 1..22: mol_type = other DNA; note = crPTPN6_45
gcgggtacttgaggtggatgat

SEQ ID NO: 892 - DNA, 20 bp, synthetic construct
  source 1..20: mol_type = other DNA; note = crPTPN6_46
caggtcaggagacagcacag

SEQ ID NO: 893 - DNA, 20 bp, synthetic construct
  source 1..20: mol_type = other DNA; note = crPTPN6_47
tgaccgctgcttcttcactt

SEQ ID NO: 894 - DNA, 20 bp, synthetic construct
  source 1..20: mol_type = other DNA; note = crPTPN6_48
tgaccgctgcttcttcactt

SEQ ID NO: 895 - DNA, 20 bp, synthetic construct
  source 1..20: mol_type = other DNA; note = crPTPN6_49
aagagctgtaccatggccac

SEQ ID NO: 896 - DNA, 20 bp, synthetic construct
  source 1..20: mol_type = other DNA; note = crPTPN6_50
aagagctgtaccatggccac

SEQ ID NO: 897 - DNA, 20 bp, synthetic construct
  source 1..20: mol_type = other DNA; note = crPTPN6_51
aagagctgtaccatggccac

SEQ ID NO: 898 - DNA, 19 bp, synthetic construct
  source 1..19: mol_type = other DNA; note = crPTPN6_52
ggaggggatggagggtagg

SEQ ID NO: 899 - DNA, 20 bp, synthetic construct
  source 1..20: mol_type = other DNA; note = crPTPN6_53
actgagcacagaaagcacga

SEQ ID NO: 900 - DNA, 20 bp, synthetic construct
  source 1..20: mol_type = other DNA; note = crTIGIT_1
gtggcatgctcttggagtct

SEQ ID NO: 901 - DNA, 20 bp, synthetic construct
  source 1..20: mol_type = other DNA; note = crTIGIT_2
ttctagtcaacgcgaccacc

SEQ ID NO: 902 - DNA, 20 bp, synthetic construct
  source 1..20: mol_type = other DNA; note = crTIGIT_3
tctcccagtgtacgtcccat

SEQ ID NO: 903 - DNA, 20 bp, synthetic construct
  source 1..20: mol_type = other DNA; note = crTIGIT_4
gaaggatggggagatgtgcc

SEQ ID NO: 904 - DNA, 20 bp, synthetic construct
  source 1..20: mol_type = other DNA; note = crTIGIT_5
tctcccagtgtacgtcccat

SEQ ID NO: 905 - DNA, 20 bp, synthetic construct
  source 1..20: mol_type = other DNA; note = crTIGIT_6
gtggcatgctcttggagtct

SEQ ID NO: 906 - DNA, 20 bp, synthetic construct
  source 1..20: mol_type = other DNA; note = crTIGIT_7
tctcccagtgtacgtcccat

SEQ ID NO: 907 - DNA, 20 bp, synthetic construct
  source 1..20: mol_type = other DNA; note = crTIGIT_8
tctcccagtgtacgtcccat

SEQ ID NO: 908 - DNA, 20 bp, synthetic construct
  source 1..20: mol_type = other DNA; note = crTIGIT_9
tgctgtgcagtgtttcagga

SEQ ID NO: 909 - DNA, 20 bp, synthetic construct
  source 1..20: mol_type = other DNA; note = crTIGIT_10
tgctgtgcagtgtttcagga

SEQ ID NO: 910 - DNA, 20 bp, synthetic construct
  source 1..20: mol_type = other DNA; note = crTIGIT_11
tgctgtgcagtgtttcagga

SEQ ID NO: 911 - DNA, 20 bp, synthetic construct
  source 1..20: mol_type = other DNA; note = crTIGIT_12
tgctgtgcagtgtttcagga

SEQ ID NO: 912 - DNA, 20 bp, synthetic construct
  source 1..20: mol_type = other DNA; note = crTIGIT_13
gccggagccattacctttct

SEQ ID NO: 913 - DNA, 20 bp, synthetic construct
  source 1..20: mol_type = other DNA; note = crTIGIT_14
tctgggtctctctctgggtg

SEQ ID NO: 914 - DNA, 20 bp, synthetic construct
  source 1..20: mol_type = other DNA; note = crTIGIT_15
tctgggtctctctctgggtg

SEQ ID NO: 915 - DNA, 20 bp, synthetic construct
  source 1..20: mol_type = other DNA; note = crTIGIT_16
ccattcctcctgtccagctg

SEQ ID NO: 916 - DNA, 20 bp, synthetic construct
  source 1..20: mol_type = other DNA; note = crTIGIT_17
ccattcctcctgtccagctg

SEQ ID NO: 917 - DNA, 22 bp, synthetic construct
  source 1..22: mol_type = other DNA; note = crTIGIT_18
catgcagctcggcacagtcctc

SEQ ID NO: 918 - DNA, 22 bp, synthetic construct
  source 1..22: mol_type = other DNA; note = crTIGIT_19
catgcagctcggcacagtcctc

SEQ ID NO: 919 - DNA, 22 bp, synthetic construct
  source 1..22: mol_type = other DNA; note = crTIGIT_20
catgcagctcggcacagtcctc

SEQ ID NO: 920 - DNA, 22 bp, synthetic construct
  source 1..22: mol_type = other DNA; note = crTIGIT_21
catgcagctcggcacagtcctc

SEQ ID NO: 921 - DNA, 20 bp, synthetic construct
  source 1..20: mol_type = other DNA; note = crTIGIT_22
tgcagttacccaggcttctg

SEQ ID NO: 922 - DNA, 20 bp, synthetic construct
  source 1..20: mol_type = other DNA; note = crTIGIT_23
agaagatgcctctggttgct

SEQ ID NO: 923 - DNA, 20 bp, synthetic construct
  source 1..20: mol_type = other DNA; note = crTIGIT_24
tggtggaggagaggtgacat

SEQ ID NO: 924 - DNA, 20 bp, synthetic construct
  source 1..20: mol_type = other DNA; note = crTIGIT_25
cgcagcactgatggagagta

SEQ ID NO: 925 - DNA, 20 bp, synthetic construct
  source 1..20: mol_type = other DNA; note = crTIGIT_26
cgcagcactgatggagagta

SEQ ID NO: 926 - DNA, 20 bp, synthetic construct
  source 1..20: mol_type = other DNA; note = crTIGIT_27
tggtggaggagaggtgacat

SEQ ID NO: 927 - DNA, 20 bp, synthetic construct
  source 1..20: mol_type = other DNA; note = crTIGIT_28
gaaggatggggagatgtgcc

SEQ ID NO: 928 - DNA, 20 bp, synthetic construct
  source 1..20: mol_type = other DNA; note = crTIGIT_29
gaaggatggggagatgtgcc

SEQ ID NO: 929 - DNA, 20 bp, synthetic construct
  source 1..20: mol_type = other DNA; note = crTIGIT_30
gaaggatggggagatgtgcc

SEQ ID NO: 930 - DNA, 20 bp, synthetic construct
  source 1..20: mol_type = other DNA; note = crTIGIT_31
tctcccagtgtacgtcccat

SEQ ID NO: 931 - DNA, 20 bp, synthetic construct
  source 1..20: mol_type = other DNA; note = crTIM3_1
gcggctactgctcatgtgat

SEQ ID NO: 932 - DNA, 20 bp, synthetic construct
  source 1..20: mol_type = other DNA; note = crTIM3_2
gacattagccaaggtcaccc

SEQ ID NO: 933 - DNA, 20 bp, synthetic construct
  source 1..20: mol_type = other DNA; note = crTIM3_3
gcggctactgctcatgtgat

SEQ ID NO: 934 - DNA, 20 bp, synthetic construct
  source 1..20: mol_type = other DNA; note = crTIM3_4
acattagccaaggtcacccc

SEQ ID NO: 935 - DNA, 20 bp, synthetic construct
  source 1..20: mol_type = other DNA; note = crTIM3_5
atgcctatctgccctgcttc

SEQ ID NO: 936 - DNA, 20 bp, synthetic construct
  source 1..20: mol_type = other DNA; note = crTIM3_6
gcggctactgctcatgtgat

SEQ ID NO: 937 - DNA, 20 bp, synthetic construct
  source 1..20: mol_type = other DNA; note = crTIM3_7
atgcagggtcctcagaagtg

SEQ ID NO: 938 - DNA, 20 bp, synthetic construct
  source 1..20: mol_type = other DNA; note = crTIM3_8
atgcctatctgccctgcttc

SEQ ID NO: 939 - DNA, 20 bp, synthetic construct
  source 1..20: mol_type = other DNA; note = crTIM3_9
atgcctatctgccctgcttc

SEQ ID NO: 940 - DNA, 20 bp, synthetic construct
  source 1..20: mol_type = other DNA; note = crTIM3_10
atgcctatctgccctgcttc

SEQ ID NO: 941 - DNA, 20 bp, synthetic construct
  source 1..20: mol_type = other DNA; note = crTIM3_11
atgcctatctgccctgcttc

SEQ ID NO: 942 - DNA, 20 bp, synthetic construct
  source 1..20: mol_type = other DNA; note = crTIM3_12
atgcctatctgccctgcttc

SEQ ID NO: 943 - DNA, 20 bp, synthetic construct
  source 1..20: mol_type = other DNA; note = crTIM3_13
ggagcctgtcctgtgtttga

SEQ ID NO: 944 - DNA, 20 bp, synthetic construct
  source 1..20: mol_type = other DNA; note = crTIM3_14
ggagcctgtcctgtgtttga

SEQ ID NO: 945 - DNA, 20 bp, synthetic construct
  source 1..20: mol_type = other DNA; note = crTIM3_15
cgcaaaggagatgtgtccct

SEQ ID NO: 946 - DNA, 20 bp, synthetic construct
  source 1..20: mol_type = other DNA; note = crTIM3_16
gcggctactgctcatgtgat

SEQ ID NO: 947 - DNA, 20 bp, synthetic construct
  source 1..20: mol_type = other DNA; note = crTIM3_17
cgcaaaggagatgtgtccct

SEQ ID NO: 948 - DNA, 20 bp, synthetic construct
  source 1..20: mol_type = other DNA; note = crTIM3_18
cgcaaaggagatgtgtccct

SEQ ID NO: 949 - DNA, 20 bp, synthetic construct
  source 1..20: mol_type = other DNA; note = crTIM3_19
cagtgggatctactgctgcc

SEQ ID NO: 950 - DNA, 20 bp, synthetic construct
  source 1..20: mol_type = other DNA; note = crTIM3_20
acgtaggtatccaggcaggt

SEQ ID NO: 951 - DNA, 20 bp, synthetic construct
  source 1..20: mol_type = other DNA; note = crTIM3_21
acgtaggtatccaggcaggt

SEQ ID NO: 952 - DNA, 20 bp, synthetic construct
  source 1..20: mol_type = other DNA; note = crTIM3_22
aggtttggaagctgagggtg

SEQ ID NO: 953 - DNA, 20 bp, synthetic construct
  source 1..20: mol_type = other DNA; note = crTIM3_23
cttgctgcccctttgattcc

SEQ ID NO: 954 - DNA, 22 bp, synthetic construct
  source 1..22: mol_type = other DNA; note = crTIM3_24
tgtttctgacattagccaaggt

SEQ ID NO: 955 - DNA, 20 bp, synthetic construct
  source 1..20: mol_type = other DNA; note = crTIM3_25
gcggctactgctcatgtgat

SEQ ID NO: 956 - DNA, 25 bp, synthetic construct
  source 1..25: mol_type = other DNA; note = crTIM3_26
cggagtagaattcatttcaaatagg

SEQ ID NO: 957 - DNA, 25 bp, synthetic construct
  source 1..25: mol_type = other DNA; note = crTIM3_27
cggagtagaattcatttcaaatagg

SEQ ID NO: 958 - DNA, 24 bp, synthetic construct
  source 1..24: mol_type = other DNA; note = crTIM3_28
tcctctctctctctctctctctct

SEQ ID NO: 959 - DNA, 20 bp, synthetic construct
  source 1..20: mol_type = other DNA; note = crTIM3_29
aggactcagccatcctgtga

SEQ ID NO: 960 - DNA, 20 bp, synthetic construct
  source 1..20: mol_type = other DNA; note = crTIM3_30
aggactcagccatcctgtga

SEQ ID NO: 961 - DNA, 20 bp, synthetic construct
  source 1..20: mol_type = other DNA; note = crTIM3_31
cagccatcctgtgatgttgt

SEQ ID NO: 962 - DNA, 20 bp, synthetic construct
  source 1..20: mol_type = other DNA; note = crTIM3_32
tggccaatgacttacgggac

SEQ ID NO: 963 - DNA, 20 bp, synthetic construct
  source 1..20: mol_type = other DNA; note = crTIM3_33
tggccaatgacttacgggac

SEQ ID NO: 964 - DNA, 20 bp, synthetic construct
  source 1..20: mol_type = other DNA; note = crTIM3_34
gcgtgcttccagtgaaccta

SEQ ID NO: 965 - DNA, 20 bp, synthetic construct
  source 1..20: mol_type = other DNA; note = crTIM3_35
gcgtgcttccagtgaaccta

SEQ ID NO: 966 - DNA, 20 bp, synthetic construct
  source 1..20: mol_type = other DNA; note = crTIM3_36
gcggctactgctcatgtgat

SEQ ID NO: 967 - DNA, 20 bp, synthetic construct
  source 1..20: mol_type = other DNA; note = crTIM3_37
gcgtgcttccagtgaaccta

SEQ ID NO: 968 - DNA, 20 bp, synthetic construct
  source 1..20: mol_type = other DNA; note = crTIM3_38
gcgtgcttccagtgaaccta

SEQ ID NO: 969 - DNA, 20 bp, synthetic construct
  source 1..20: mol_type = other DNA; note = crTIM3_39
gcgtgcttccagtgaaccta

SEQ ID NO: 970 - DNA, 20 bp, synthetic construct
  source 1..20: mol_type = other DNA; note = crTIM3_40
gctgtctctttgggaaagcc

SEQ ID NO: 971 - DNA, 20 bp, synthetic construct
  source 1..20: mol_type = other DNA; note = crTIM3_41
gctgtctctttgggaaagcc

SEQ ID NO: 972 - DNA, 20 bp, synthetic construct
  source 1..20: mol_type = other DNA; note = crTIM3_42
gctgtctctttgggaaagcc

SEQ ID NO: 973 - DNA, 20 bp, synthetic construct
  source 1..20: mol_type = other DNA; note = crTIM3_43
ccgtgttacctgggaaatgc

SEQ ID NO: 974 - DNA, 20 bp, synthetic construct
  source 1..20: mol_type = other DNA; note = crTIM3_44
ccgtgttacctgggaaatgc

SEQ ID NO: 975 - DNA, 20 bp, synthetic construct
  source 1..20: mol_type = other DNA; note = crTIM3_45
ccgtgttacctgggaaatgc

SEQ ID NO: 976 - DNA, 20 bp, synthetic construct
  source 1..20: mol_type = other DNA; note = crTIM3_46
ccgtgttacctgggaaatgc

SEQ ID NO: 977 - DNA, 20 bp, synthetic construct
  source 1..20: mol_type = other DNA; note = crTIM3_47
agtggaggagcccaatgagt

SEQ ID NO: 978 - DNA, 20 bp, synthetic construct
  source 1..20: mol_type = other DNA; note = crTIM3_48
agtggaggagcccaatgagt

SEQ ID NO: 979 - DNA, 20 bp, synthetic construct
  source 1..20: mol_type = other DNA; note = crTIM3_49
aacaggactgcagcagtagc

SEQ ID NO: 980 - DNA, 20 bp, synthetic construct
  source 1..20: mol_type = other DNA; note = crTIM3_50
atgcagggtcctcagaagtg

SEQ ID NO: 981 - DNA, 20 bp, synthetic construct
  source 1..20: mol_type = other DNA; note = crTIM3_51
atgcagggtcctcagaagtg

SEQ ID NO: 982 - DNA, 20 bp, synthetic construct
  source 1..20: mol_type = other DNA; note = crAAVS1
aagaggatggagaggtggct

SEQ ID NO: 983 - DNA, 21 bp, synthetic construct
  source 1..21: mol_type = other DNA; note = CD247_1_OT1
acaacagccatcctgcaggca

SEQ ID NO: 984 - DNA, 21 bp, synthetic construct
  source 1..21: mol_type = other DNA; note = CD247_3_OT2
ggatccagcaaggcaacgctc

SEQ ID NO: 985 - DNA, 21 bp, synthetic construct
  source 1..21: mol_type = other DNA; note = CD247_3_OT3
ggatgcagcaggccaagggtc

SEQ ID NO: 986 - DNA, 21 bp, synthetic construct
  source 1..21: mol_type = other DNA; note = CD247_3_OT4
ggatccagcatgccaaggctt

SEQ ID NO: 987 - DNA, 21 bp, synthetic construct
  source 1..21: mol_type = other DNA; note = CD247_3_OT5
ggatccagcatgccaaggctt

SEQ ID NO: 988 - DNA, 21 bp, synthetic construct
  source 1..21: mol_type = other DNA; note = CTLA4_2_OT1
cagaagacagtgaggaagagg

SEQ ID NO: 989 - DNA, 21 bp, synthetic construct
  source 1..21: mol_type = other DNA; note = CTLA4_2_OT2
cagaagacaggtgtgatgaga

SEQ ID NO: 990 - DNA, 21 bp, synthetic construct
  source 1..21: mol_type = other DNA; note = CTLA4_2_OT3
cagaagacaggtgtgatgaga

SEQ ID NO: 991 - DNA, 21 bp, synthetic construct
  source 1..21: mol_type = other DNA; note = CTLA4_2_OT4
cagaagaaagggagggagaga

SEQ ID NO: 992 - DNA, 21 bp, synthetic construct
  source 1..21: mol_type = other DNA; note = CTLA4_2_OT5
cagaagagagggaagaagaaa

SEQ ID NO: 993 - DNA, 21 bp, synthetic construct
  source 1..21: mol_type = other DNA; note = CTLA4_2_OT6
tagaacacagggataaagaga

SEQ ID NO: 994 - DNA, 21 bp, synthetic construct
  source 1..21: mol_type = other DNA; note = CTLA4_2_OT7
cagaagatacagatgaagaga

SEQ ID NO: 995 - DNA, 21 bp, synthetic construct
  source 1..21: mol_type = other DNA; note = CTLA4_2_OT8
caaaagacatggatggagaga

SEQ ID NO: 996 - DNA, 21 bp, synthetic construct
  source 1..21: mol_type = other DNA; note = CTLA4_2_OT9
cagaacacaggaataaagaga

SEQ ID NO: 997 - DNA, 21 bp, synthetic construct
  source 1..21: mol_type = other DNA; note = CTLA4_5_OT10
agagggccaaggctcagctga

SEQ ID NO: 998 - DNA, 21 bp, synthetic construct
  source 1..21: mol_type = other DNA; note = CTLA4_5_OT11
aggggaacaaggctcagctca

SEQ ID NO: 999 - DNA, 21 bp, synthetic construct
  source 1..21: mol_type = other DNA; note = CTLA4_5_OT12
agcggtccaagcctcagctga

SEQ ID NO: 1000 - DNA, 21 bp, synthetic construct
  source 1..21: mol_type = other DNA; note = CTLA4_5_OT13
agaggcccaaggctcagctca

SEQ ID NO: 1001 - DNA, 21 bp, synthetic construct
  source 1..21: mol_type = other DNA; note = TIGIT_1_OT1
aggccttacgtgaggtgtggg

SEQ ID NO: 1002 - DNA, 21 bp, synthetic construct
  source 1..21: mol_type = other DNA; note = TIGIT_1_OT2
aggccttgcctgatgccaggg

SEQ ID NO: 1003 - DNA, 21 bp, synthetic construct
  source 1..21: mol_type = other DNA; note = TIGIT_2_OT3
ttcctccctctagtggcttaa

SEQ ID NO: 1004 - DNA, 21 bp, synthetic construct
  source 1..21: mol_type = other DNA; note = TIGIT_2_OT4
gtccaccctctattgactgag

SEQ ID NO: 1005 - DNA, 21 bp, synthetic construct
  source 1..21: mol_type = other DNA; note = TIGIT_2_OT5
gtgctccctccagtggctcag

SEQ ID NO: 1006 - DNA, 21 bp, synthetic construct
  source 1..21: mol_type = other DNA; note = TIGIT_2_OT6
gttctgcctctagtggatgag

SEQ ID NO: 1007 - DNA, 21 bp, synthetic construct
  source 1..21: mol_type = other DNA; note = TIM3_1_OT1
ctagtaagtggtagcaccagc

SEQ ID NO: 1008 - DNA, 21 bp, synthetic construct
  source 1..21: mol_type = other DNA; note = TIM3_1_OT2
gttttaaatagtagcagcagc

SEQ ID NO: 1009 - DNA, 21 bp, synthetic construct
  source 1..21: mol_type = other DNA; note = TIM3_1_OT3
cttataagtagtaggagcagt

SEQ ID NO: 1010 - DNA, 21 bp, synthetic construct
  source 1..21: mol_type = other DNA; note = TIM3_1_OT4
cttaaaagcagtagcagcagc

SEQ ID NO: 1011 - DNA, 21 bp, synthetic construct
  source 1..21: mol_type = other DNA; note = TIM3_1_OT5
ctcataagtagtagcagcagc

SEQ ID NO: 1012 - DNA, 21 bp, synthetic construct
  source 1..21: mol_type = other DNA; note = TIM3_1_OT6
cttttaagtagcaacagcagc

SEQ ID NO: 1013 - DNA, 21 bp, synthetic construct
  source 1..21: mol_type = other DNA; note = TIM3_1_OT7
cttttaaagagtagcagcagc

SEQ ID NO: 1014 - DNA, 21 bp, synthetic construct
  source 1..21: mol_type = other DNA; note = TIM3_2_OT8
aaagggtgcttagcaccaggg

SEQ ID NO: 1015 - DNA, 21 bp, synthetic construct
  source 1..21: mol_type = other DNA; note = TIM3_2_OT9
aaagcatgcttaccaccaggc

SEQ ID NO: 1016 - DNA, 21 bp, synthetic construct
  source 1..21: mol_type = other DNA; note = PDCD1_1_OT1
gcacgaagcactccgctgtgc

SEQ ID NO: 1017 - DNA, 21 bp, synthetic construct
  source 1..21: mol_type = other DNA; note = PDCD1_1_OT2
gcaatgagctctcagatgtgt

SEQ ID NO: 1018 - DNA, 21 bp, synthetic construct
  source 1..21: mol_type = other DNA; note = PDCD1_1_OT3
gaaggaagctctccagtgtgt

SEQ ID NO: 1019 - DNA, 21 bp, synthetic construct
  source 1..21: mol_type = other DNA; note = PDCD1_1_OT4
ccacgaagctctctgaaatgt

SEQ ID NO: 1020 - DNA, 21 bp, synthetic construct
  source 1..21: mol_type = other DNA; note = PDCD1_2_OT1
tgttcagagacaatagaagcc

SEQ ID NO: 1021 - DNA, 21 bp, synthetic construct
  source 1..21: mol_type = other DNA; note = PDCD1_2_OT2
tctgcagggacattgagagct

SEQ ID NO: 1022 - DNA, 21 bp, synthetic construct
  source 1..21: mol_type = other DNA; note = PDCD1_2_OT3
tctgcaaggtcaacaggaacc

SEQ ID NO: 1023 - DNA, 21 bp, synthetic construct
  source 1..21: mol_type = other DNA; note = PDCD1_2_OT4
tctgcagggacaaaatcaggc

SEQ ID NO: 1024 - DNA, 21 bp, synthetic construct
  source 1..21: mol_type = other DNA; note = PDCD1_2_OT5
tcatcagagacaatagaagcc

SEQ ID NO: 1025 - DNA, 21 bp, synthetic construct
  source 1..21: mol_type = other DNA; note = PDCD1_2_OT6
actgcagagactataggatcc

SEQ ID NO: 1026 - DNA, 21 bp, synthetic construct
  source 1..21: mol_type = other DNA; note = PDCD1_2_OT7
tctgcagggaatattgcagcc

SEQ ID NO: 1027 - DNA, 21 bp, synthetic construct
  source 1..21: mol_type = other DNA; note = PDCD1_2_OT8
tctggaaggtcaaaaggagcc

SEQ ID NO: 1028 - DNA, 21 bp, synthetic construct
  source 1..21: mol_type = other DNA; note = PDCD1_2_OT9
tctgcaggaaccacaggaacc

SEQ ID NO: 1029 - DNA, 21 bp, synthetic construct
  source 1..21: mol_type = other DNA; note = PDCD1_2_OT10
tctgcaaggaaaaaaggaggc

SEQ ID NO: 1030 - DNA, 21 bp, synthetic construct
  source 1..21: mol_type = other DNA; note = PDCD1_2_OT11
tctgcaggggcaataggggaa

SEQ ID NO: 1031 - DNA, 21 bp, synthetic construct
  source 1..21: mol_type = other DNA; note = PDCD1_2_OT12
tctgaagggacagtgggatcc

SEQ ID NO: 1032 - DNA, 21 bp, synthetic construct
  source 1..21: mol_type = other DNA; note = PDCD1_2_OT13
ccagcagggacaataggagtt

SEQ ID NO: 1033 - DNA, 21 bp, synthetic construct
  source 1..21: mol_type = other DNA; note = PDCD1_2_OT14
tctccagggaccataggggct

SEQ ID NO: 1034 - DNA, 21 bp, synthetic construct
  source 1..21: mol_type = other DNA; note = PDCD1_2_OT15
tctgcagcgaagatcggagcc

SEQ ID NO: 1035 - DNA, 21 bp, synthetic construct
  source 1..21: mol_type = other DNA; note = PDCD1_2_OT16
tctgcagggagtagaggaacc

SEQ ID NO: 1036 - DNA, 21 bp, synthetic construct
  source 1..21: mol_type = other DNA; note = PDCD1_2_OT17
tctacaggggcaatcggagct

SEQ ID NO: 1037 - DNA, 21 bp, synthetic construct
  source 1..21: mol_type = other DNA; note = PDCD1_2_OT18
gctgcagggacaacctgagcc

SEQ ID NO: 1038 - DNA, 21 bp, synthetic construct
  source 1..21: mol_type = other DNA; note = PDCD1_2_OT19
actgcaggtataataggggcc

SEQ ID NO: 1039 - DNA, 21 bp, synthetic construct
  source 1..21: mol_type = other DNA; note = PDCD1_2_OT20
cctgcctggacattaggagcc

SEQ ID NO: 1040 - DNA, 21 bp, synthetic construct
  source 1..21: mol_type = other DNA; note = PDCD1_2_OT21
tctccagagccaataggagct

SEQ ID NO: 1041 - DNA, 21 bp, synthetic construct
  source 1..21: mol_type = other DNA; note = LAG3_1_OT1
gggtgcatacatgtgtgcctg

SEQ ID NO: 1042 - DNA, 21 bp, synthetic construct
  source 1..21: mol_type = other DNA; note = LAG3_2_OT2
ggtcacctggattccttgaga

SEQ ID NO: 1043 - DNA, 21 bp, synthetic construct
  source 1..21: mol_type = other DNA; note = LAG3_2_OT3
ggtcccctggagccctgcgga

SEQ ID NO: 1044 - DNA, 21 bp, synthetic construct
  source 1..21: mol_type = other DNA; note = LAG3_2_OT4
tgtcatctggacccctgggga

SEQ ID NO: 1045 - DNA, 21 bp, synthetic construct
  source 1..21: mol_type = other DNA; note = LAG3_2_OT5
ggtcagctggatccatggtga

SEQ ID NO: 1046 - DNA, 21 bp, synthetic construct
  source 1..21: mol_type = other DNA; note = PTPN11 1 OT1
aaagggtgcttagcaccaggg

SEQ ID NO: 1047 - DNA, 21 bp, synthetic construct
  source 1..21: mol_type = other DNA; note = PTPN11_2_OT2
aaagcatgcttaccaccaggc

SEQ ID NO: 1048 - DNA, 21 bp, synthetic construct
  source 1..21: mol_type = other DNA; note = PTPN6_1_OT1
tatgacctgccttgaggggag

SEQ ID NO: 1049 - DNA, 21 bp, synthetic construct
  source 1..21: mol_type = other DNA; note = PTPN6_1_OT2
tatgacctgtttgctggggag

SEQ ID NO: 1050 - DNA, 21 bp, synthetic construct
  source 1..21: mol_type = other DNA; note = PTPN6_1_OT3
tatgaactgtataaaggggag

(19) United States
(12) Patent Application Publication
(10) Pub. No.: US 2025/0388896 A1
(43) Pub. Date: Dec. 25, 2025

(54) COMPOSITION AND METHODS FOR TRANSGENE INSERTION

(71) Applicants: Marina MOHR, Boulder, CO (US); Ryan T. GILL, Boulder, CO (US); Johanne GUDMAN-HØYER, Boulder, CO (US); Katrine Vildershøj Wolf ZEEBERG, Boulder, CO (US); Dominika Joanna JEDRZEJCZYK, Boulder, CO (US); Tanya WARNECKE, Boulder (CO); ARTISAN DEVELOPMENT LABS, INC, Boulder, CO (US)

(72) Inventors: Marina Mohr, Boulder, CO (US); Ryan T. Gill, Boulder, CO (US); Johanne Gudman-Høyer, Boulder, CO (US); Katrine Vildershøj Wolf Zeeberg, Boulder, CO (US); Dominika Joanna Jedrzejczyk, Boulder, CO (US); Tanya Warnecke, Boulder, CO (US)

(73) Assignee: Celyntra Therapeutics SA, Mont-Saint-Guibert (BE)

(21) Appl. No.: 18/842,408

(22) PCT Filed: Mar. 1, 2023
(86) PCT No.: PCT/US2023/014203

§ 371 Date: Aug. 29, 2024

Related U.S. Application Data
(60) Provisional application No. 63/315,483, filed on Mar. 1, 2022.

(51) Int. Cl. C12N 15/113; C12N 5/0783; C12N 9/22

(52) U.S. Cl. CPC C12N 15/113; C12N 5/0636; C12N 9/222; C12N 2310/20

(57) ABSTRACT

CRISPR-Cas systems have been engineered for various purposes, such as genomic DNA cleavage, base editing, epigenome editing, and genomic imaging. Although significant developments have been made, there still remains a need for new and useful CRISPR-Cas systems as powerful precise genome targeting tools. The invention disclosed herein comprises CRISPR-Cas based methods for high integration and expression efficiency of transgenes together with high post-transfection cell viability in eukaryotic cells.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### REFERENCE TO RELATED APPLICATIONS

This application claims the benefit of and priority to U.S. Provisional Patent Application No. 63/315,483, filed Mar. 1, 2022, the disclosure of which is hereby incorporated by reference in its entirety for all purposes.

### REFERENCE TO AN ELECTRONIC SEQUENCE LISTING

The contents of the electronic sequence listing (sequencelisting.txt; Size: 1.20 MB; and Date of Creation: May 1, 2025) is herein incorporated by reference in its entirety.

### STATEMENT AS TO BACKGROUND

CRISPR-Cas systems have been engineered for various purposes, such as genomic DNA cleavage, base editing, epigenome editing, and genomic imaging. Although significant developments have been made, there still remains a need for new and useful CRISPR-Cas systems as powerful precise genome targeting tools. The invention disclosed herein comprises CRISPR-Cas based methods for high integration and expression efficiency of transgenes together with high post-transfection cell viability in eukaryotic cells.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows a schematic representation showing the structure of an exemplary single guide type V-A CRISPR system. FIG. 1B is a schematic representation showing the structure of an exemplary dual guide type V-A CRISPR system.

FIGS. 2A-C show a series of schematic representation showing incorporation of a protecting group (e.g., a protective nucleotide sequence or a chemical modification) (FIG. 2A), a donor template-recruiting sequence (FIG. 2B), and an editing enhancer (FIG. 2C) into a type V-A CRISPR-Cas system. These additional elements are shown in the context of a dual guide type V-A CRISPR system, but it is understood that they can also be present in other CRISPR systems, including a single guide type V-A CRISPR system, a single guide type II CRISPR system, or a dual guide type II CRISPR system.

FIG. 3 shows a diagram of MAD7 comprising one or more nuclear localization signals (NLS).

FIG. 4 shows editing frequency at the DNMT1 locus in and post-transfection cell viability of T-cell leukemic cells following treatment comprising one or more guide nucleic acids complexed with MAD7 comprising one or more NLS.

FIG. 5 shows editing frequency at the DNMT1 locus in T-cell leukemic cells using multiple electroporation programs in combination with the SE electroporation buffer.

FIG. 6 shows editing frequency at the DNMT1 locus in T-cell leukemic cells using multiple electroporation programs in combination with the SF electroporation buffer.

FIG. 7 shows editing frequency at the DNMT1 locus in T-cell leukemic cells using multiple electroporation programs in combination with the SG electroporation buffer.

FIG. 8 shows editing frequency at the DNMT1 locus in T-cell leukemic cells using multiple electroporation programs.

FIG. 9 shows editing frequency by type at eight loci in T-cell leukemic cells using multiple guide nucleic acids complexed with MAD7 comprising one or more NLS.

FIG. 10 shows a comparison of editing efficiency between T-cell leukemic cells treated with MAD7 comprising one or more guide nucleic acids targeting the DNMT1 locus as compared to a control guide nucleic acid binned by editing frequency.

FIG. 11 shows editing frequency by PAM motif in T-cell leukemic cells using multiple guide nucleic acids complexed with MAD7 comprising one or more NLS.

FIG. 12A shows sequence logo plots for multiple guide nucleic acids binned by editing frequency in T-cell leukemic cells using when complexed with MAD7 comprising one or more NLS.

FIG. 12B shows nucleotide and dinucleotide frequency for multiple guide nucleic acids binned by editing frequency in T-cell leukemic cells using when complexed with MAD7 comprising one or more NLS.

FIG. 13 shows trinucleotide AAA or UUU frequency binned by editing frequency in T-cell leukemic cells following treatment with multiple guide nucleic acids complexed with MAD7 comprising one or more NLS.

FIG. 14 shows editing frequency for both INDELs and frameshift mutations at eight loci in T-cell leukemic cells following treatment with multiple guide nucleic acids complexed with MAD7 comprising one or more NLS.

FIG. 15 shows the correlation between INDEL frequency in the gNA validation experiment versus INDEL formation in the gNA screen experiment.

FIG. 16 shows the proportion of frameshift to INDELs at eight loci in T-cell leukemic cells following treatment with multiple guide nucleic acids complexed with MAD7 comprising one or more NLS.

FIG. 17 shows INDEL frequency for gNAs comprising representative spacer sequences complexed with MAD7 comprising one or more NLS in T-cell leukemic cells at predicted off-target sites.

FIG. 18 shows INDEL frequency for gNAs comprising representative spacer sequences complexed with MAD7 comprising one or more NLS in T-cell leukemic cells at predicted off-target sites.

FIG. 19 shows INDEL frequency at the AAVS1 locus in T-cell leukemic cells following treatment with a gNA:MAD7 complex.

FIG. 20 shows GFP insertion efficiency at the AAVS1 locus and cell viability following treatment for multiple primer constructs.

FIG. 21 shows GFP insertion efficiency at the AAVS1 locus with increasing concentrations of donor template (e.g., HDRT) and variable homology arm length.

FIG. 22 shows CAR insertion efficiency at the AAVS1 locus and cell viability with increasing concentrations of donor template and variable homology arm length.

FIG. 23 shows CAR insertion efficiency (A) at the AAVS1 locus and cell viability (B) in primary T-cells.

FIG. 24 illustrates an exemplary method for stabilizing nucleic acid-guided nucleases.

FIG. 25 illustrates an exemplary method for engineering a human target genome.

DETAILED DESCRIPTION
Outline

| I. | High efficiency transgene insertion | |
|---|---|---|
| II. | Engineered non-naturally-occurring dual guide CRISPR-cas | |
| | systems | |
| | A. | Cas proteins |
| | B. | Guide nucleic acids |
| | C. | gNA Modifications |
| III. | Composition and methods for targeting, editing, and/or modifying | |
| | genomic DNA | |
| | A. | Ribonucleoprotein (RNP) delivery and “cas RNA” delivery |
| | B. | CRISPR expression systems |
| | C. | Donor templates |
| | D. | Efficiency and specificity |
| | E. | Multiplex |
| IV. | Pharmaceutical compositions | |
| V. | Therapeutic uses | |
| | A. | Gene therapies |

| | |
|---|---|
| VI. | Kits |
| VII. | Embodiments |
| VIII. | Examples |
| IX. | Equivalents |

### I. HIGH EFFICIENCY TRANSGENE INSERTION

Recent advances have been made in precise genome targeting technologies. For example, specific loci in genomic DNA can be targeted, edited, or otherwise modified by designer meganucleases, zinc finger nucleases, or transcription activator-like effectors (TALEs). Furthermore, the CRISPR-Cas systems of bacterial and archaeal adaptive immunity have been adapted for precise targeting of genomic DNA in eukaryotic cells. Compared to the earlier generations of genome editing tools, the CRISPR-Cas systems are easy to set up, scalable, and amenable to targeting multiple positions within the eukaryotic genome, thereby providing a major resource for new applications in genome engineering. In certain embodiments, provided herein are compositions, methods, and/or kits for genome engineering. In certain embodiments, provided herein are compositions, methods, and/or kits for genome engineering of eukaryotic cells. In certain embodiments, provided herein are compositions, methods, and/or kits for genome engineering of human cells. In certain embodiments, provided herein are compositions, methods, and/or kits for genome engineering of human immune or stem cells. In certain embodiments, provided herein are compositions, methods, and/or kits for efficient genome engineering. In certain embodiments, provided herein are compositions, methods, and/or kits for efficient genome engineering via optimized compositions and/or methods. In certain embodiments, provided herein are compositions, methods, and/or kits comprising nucleases. In certain embodiments, provided herein are compositions, methods, and/or kits comprising nucleic acid-guided nucleases, e.g., CRISPR-cas nucleases. In certain embodiments, provided herein are compositions, methods, and/or kits comprising guide nucleic acids (gNAs). In certain embodiments, provided herein are compositions, methods, and/or kits comprising molecules that improve the efficiency of genome editing. In certain embodiments, provided herein are compositions, methods, and/or kits comprising molecules that stabilize RNPs, e.g., RNP stabilizer. In certain embodiments, provided herein are compositions, methods, and/or kits comprising molecules that inhibit non-homologous end joining (NHEJ), e.g., NHEJ inhibitor. In certain embodiments, provided herein are compositions, methods, and/or kits comprising improved combinations and/or concentrations of one or more of the following items: (1) one or more guide nucleic acids (gNA), (2) one or more nucleases, (3) one or more donor templates, (4) one or more RNP stabilizers, (5) one or more NHEJ inhibitors, (6) one or more cell growth and/or recovery mediums, and/or (7) one or more human target cells.

In certain embodiments, provided herein are compositions, methods, and/or kits comprising at least one of the seven items. In certain embodiments, provided herein are compositions, methods, and/or kits comprising at least two of the seven items. In certain embodiments, provided herein are compositions, methods, and/or kits comprising at least three of the seven items. In certain embodiments, provided herein are compositions, methods, and/or kits comprising at least four of the seven items. In certain embodiments, provided herein are compositions, methods, and/or kits comprising at least five of the seven items. In certain embodiments, provided herein are compositions, methods, and/or kits comprising at least six of the seven items. In certain embodiments, provided herein are compositions, methods, and/or kits comprising all seven items.

In certain embodiments, provided herein are compositions, methods, and/or kits comprising one or more nucleic acid guided nucleases, i.e., nuclease. In certain embodiments, provided herein are compositions, methods, and/or kits comprising one or more nucleases that further comprise at least one of the six additional items. In certain embodiments, provided herein are compositions, methods, and/or kits comprising one or more nucleases that further comprise at least two of the six additional items. In certain embodiments, provided herein are compositions, methods, and/or kits comprising one or more nucleases that further comprise at least three of the six additional items. In certain embodiments, provided herein are compositions, methods, and/or kits comprising one or more nucleases that further comprise at least four of the six additional items. In certain embodiments, provided herein are compositions, methods, and/or kits comprising one or more nucleases that further comprise at least five of the six additional items. In certain embodiments, provided herein are compositions, methods, and/or kits comprising one or more nucleases that further comprise all six additional items.

In certain embodiments, provided herein are compositions, methods, and/or kits comprising one or more guide nucleic acids. In certain embodiments, provided herein are compositions, methods, and/or kits comprising one or more guide nucleic acids that further comprise at least one of the six additional items. In certain embodiments, provided herein are compositions, methods, and/or kits comprising one or more guide nucleic acids that further comprise at least two of the six additional items. In certain embodiments, provided herein are compositions, methods, and/or kits comprising one or more guide nucleic acids that further comprise at least three of the six additional items. In certain embodiments, provided herein are compositions, methods, and/or kits comprising one or more guide nucleic acids that further comprise at least four of the six additional items. In certain embodiments, provided herein are compositions, methods, and/or kits comprising one or more guide nucleic acids that further comprise at least five of the six additional items. In certain embodiments, provided herein are compositions, methods, and/or kits comprising one or more guide nucleic acids that further comprise all six additional items.

In certain embodiments, provided herein are compositions, methods, and/or kits comprising one or more guide nucleic acids and one or more nucleases. In certain embodiments, provided herein are compositions, methods, and/or kits comprising one or more guide nucleic acids and one or more nucleases that further comprise at least one of the five additional items. In certain embodiments, provided herein are compositions, methods, and/or kits comprising one or more guide nucleic acids and one or more nucleases that further comprise at least two of the five additional items. In certain embodiments, provided herein are compositions, methods, and/or kits comprising one or more guide nucleic acids and one or more nucleases that further comprise at least three of the five additional items. In certain embodiments, provided herein are compositions, methods, and/or kits comprising one or more guide nucleic acids and one or more nucleases that further comprise at least four of the five additional items. In certain embodiments, provided herein are compositions, methods, and/or kits comprising one or more guide nucleic acids and one or more nucleases that further comprise all five additional items.

In certain embodiments, provided herein are compositions, methods, and/or kits comprising one or more guide nucleic acids and one or more RNP stabilizers. In certain embodiments, provided herein are compositions, methods, and/or kits comprising one or more guide nucleic acids and one or more RNP stabilizers that further comprise at least one of the five additional items. In certain embodiments, provided herein are compositions, methods, and/or kits comprising one or more guide nucleic acids and one or more RNP stabilizers that further comprise at least two of the five additional items. In certain embodiments, provided herein are compositions, methods, and/or kits comprising one or more guide nucleic acids and one or more RNP stabilizers that further comprise at least three of the five additional items. In certain embodiments, provided herein are compositions, methods, and/or kits comprising one or more guide nucleic acids and one or more RNP stabilizers that further comprise at least four of the five additional items. In certain embodiments, provided herein are compositions, methods, and/or kits comprising one or more guide nucleic acids and one or more RNP stabilizers that further comprise all five additional items.

In certain embodiments, provided herein are compositions, methods, and/or kits comprising one or more guide nucleic acids, one or more RNP stabilizers, and one or more nucleases. In certain embodiments, provided herein are compositions, methods, and/or kits comprising one or more guide nucleic acids, one or more RNP stabilizers, and one or more nucleases that further comprise at least one of the four additional items. In certain embodiments, provided herein are compositions, methods, and/or kits comprising one or more guide nucleic acids, one or more RNP stabilizers, and one or more nucleases that further comprise at least two of the four additional items. In certain embodiments, provided herein are compositions, methods, and/or kits comprising one or more guide nucleic acids, one or more RNP stabilizers, and one or more nucleases that further comprise at least three of the four additional items. In certain embodiments, provided herein are compositions, methods, and/or kits comprising one or more guide nucleic acids, one or more RNP stabilizers, and one or more nucleases that further comprise all four additional items.

In certain embodiments, provided herein are compositions, methods, and/or kits comprising one or more human target cells. In certain embodiments, provided herein are compositions, methods, and/or kits comprising one or more human target cells that further comprise at least one of the six additional items. In certain embodiments, provided herein are compositions, methods, and/or kits comprising one or more human target cells that further comprise at least two of the six additional items. In certain embodiments, provided herein are compositions, methods, and/or kits comprising one or more human target cells that further comprise at least three of the six additional items. In certain embodiments, provided herein are compositions, methods, and/or kits comprising one or more human target cells that further comprise at least four of the six additional items. In certain embodiments, provided herein are compositions, methods, and/or kits comprising one or more human target cells that further comprise at least five of the six additional items. In certain embodiments, provided herein are compositions, methods, and/or kits comprising one or more human target cells that further comprise all six additional items.

In certain embodiments, provided herein are compositions, methods, and/or kits comprising one or more human target cells and one or more NHEJ inhibitor. In certain embodiments, provided herein are compositions, methods, and/or kits comprising one or more human target cells and one or more NHEJ inhibitor that further comprise at least one of the five additional items. In certain embodiments, provided herein are compositions, methods, and/or kits comprising one or more human target cells and one or more NHEJ inhibitor that further comprise at least two of the five additional items. In certain embodiments, provided herein are compositions, methods, and/or kits comprising one or more human target cells and one or more NHEJ inhibitor that further comprise at least three of the five additional items. In certain embodiments, provided herein are compositions, methods, and/or kits comprising one or more human target cells and one or more NHEJ inhibitor that further comprise at least four of the five additional items. In certain embodiments, provided herein are compositions, methods, and/or kits comprising one or more human target cells and one or more NHEJ inhibitor that further comprise all five additional items.

In certain embodiments, provided herein are compositions, methods, and/or kits comprising one or more guide nucleic acids, one or more nucleases, and one or more human target cells. In certain embodiments, provided herein are compositions, methods, and/or kits comprising one or more guide nucleic acids, one or more nucleases, and one or more human target cells that further comprise at least one of the four additional items. In certain embodiments, provided herein are compositions, methods, and/or kits comprising one or more guide nucleic acids, one or more nucleases, and one or more human target cells that further comprise at least two of the four additional items. In certain embodiments, provided herein are compositions, methods, and/or kits comprising one or more guide nucleic acids, one or more nucleases, and one or more human target cells that further comprise at least three of the four additional items. In certain embodiments, provided herein are compositions, methods, and/or kits comprising one or more guide nucleic acids, one or more nucleases, and one or more human target cells that further comprise all four additional items. In certain embodiments comprising one or more nucleases, and one or more human target cells, the compositions, methods, and/or kits further can comprise one or more RNP stabilizers, one or more donor templates, and/or one or more NHEJ inhibitors

In certain embodiments, provide herein are compositions, methods, and/or kits wherein the optimized combinations and/or concentrations, e.g., condition and/or treatment, of gNA, nuclease, donor template, RNP stabilizers, and/or NHEJ inhibitors result in at least 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.25, 2.5, 2.75, 3, 4, 5, 6, 7, 8, or 9-fold and/or not more than 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.25, 2.5, 2.75, 3, 4, 5, 6, 7, 8, 9, or 10-fold increased editing via homology directed repair (HDR) as compared to editing via NHEJ, for example 1.1-10-fold increased editing, preferably 1.1-5-fold increased editing, even more preferably 1.1-3-fold increased editing, yet more preferably 1.1-2-fold increased editing.

In certain embodiments, provided herein are compositions, methods, and/or kits comprising one or more additives that stabilize RNPs, e.g., RNP stabilizer. In certain embodiments, the one or more additives that stabilize RNPs are combined with the nuclease and the guide nucleic acid. In certain embodiments, the one or more additives that stabilize RNPs are combined with the guide nucleic acid prior to combination with the nuclease. In certain embodiments, the one or more additives that stabilize RNPs are combined with the nuclease prior to combination with the guide nucleic acid. In certain embodiments, the one or more additives that stabilize RNPs are combined with the pre-formed RNP complex comprising one or more nucleases and a guide nucleic acid. In certain embodiments, the one or more additives that stabilize RNPs prevent aggregation and/or support dispersion of RNP complexes in a population of RNPs.

In certain embodiments, an RNP stabilizer may comprise any suitable protein stabilizer, such as a protein stabilizer known in the art. In certain embodiments, an RNP stabilizer comprises 1,2,3-heptanetriol, 2-Amino-2-(hydroxymethyl)-1,3-propanediol (Tris), 3-(1-pyridino)-1-propane sulfonate (NDSB 201), 3-[(3-cholamidopropyl)dimethylammonio]-1-propanesulfonate (CHAPS), 6-aminocaproic acid, adenosine diphosphate (ADP), adenosine triphosphate (ATP), alpha-cyclodextrin, amidosulfobetaine-14 (ASB-14), ammonium acetate, ammonium nitrate, ammonium sulfate, arginine, arginine ethylester, barium chloride, barium iodide, benzamidine HCl, beta-cyclodextrin, beta-mercaptoethanol (BME), biotin, calcium chloride, cesium chloride, cesium sulfate, cetyltrimethylammonium bromide (CTAB), choline chloride, citric acid, cobalt chloride, copper (II) chloride, cyclohexanol, D-sorbitol, dimethylethylammoniumpropane sulfonate (NDSB 195), dithiothreitol (DTT), erythritol, ethanol, ethylene glycol, ethylene glycol-bis(βbeta-aminoethyl ether)-N,N,N′,N′-tetraacetic acid (EGTA), ethylenediaminetetraacetic acid (EDTA), formamide, gadolinium bromide, gamma butyrolactone, glucose, glutamic acid, glutamine, glycerol, glycine, glycine betaine, glycine-glycine-glycine, guanidine HCl, guanosine triphosphate (GTP), holmium chloride, imidazole, iron (III) chloride, Jeffamine M-600, lanthanum acetate, lauryl sulfobetaine, lauryldimethylamine N-oxide (LDAO), lithium sulfate, magnesium chloride, magnesium sulfate, manganese chloride, mannitol, N-(2-hydroxyethyl) piperazine-N′-(3-propanesulfonic acid) (EPPS), N-dodecyl beta-D-maltoside (DDM), N-ethylurea, n-hexanol, N-lauryl sarcoside, N-lauryl sarcosine, N-methylformamide, N-methylurea, n-octyl-b-D-glucoside (OG: Octyl glucoside), n-penthanol, nickel chloride, non-detergent sulfo betaine (NDSB), Nonidet P40 (NP40), octyl beta-D-glucopyranoside, poly-L-glutamic acid, polyethylene glycol (for example, PEG 300, PEG 3350, PEG 4000), polyethyleneglycol lauryl ether (Brij 35), polyoxyethylene (2) oleyl ether (Brij 93), polyoxyethylene cetyl ether (Brij 56), polyvinylpyrrolidone 40 (PVP40), potassium chloride, potassium citrate, potassium nitrate, proline, putrescine, spermidine, spermine, riboflavin, samarium bromide, sarcosine, sodium acetate, sodium chloride, sodium dodecyl sulfate (SDS), sodium fluoride, sodium iodide, sodium lauroyl sarcosinate (Sarkosyl), sodium malonate, sodium molybdate, sodium selenite, sodium sulfate, sodium thiocyanate, sucrose, taurine, trehalose, tricine, triethylamine, trimethylamine N-oxide (TMAO), tris(2-carboxyethyl)phosphine (TCEP), Triton X-100, Tween 20, Tween 60, Tween 80, urea, vitamin B12, xylitol, yttrium chloride, yttrium nitrate, zinc chloride, Zwittergent 3-08, Zwittergent 3-14, or a combination thereof. In certain embodiments, the RNP stabilizer comprises a negatively charged polymer. In certain embodiments, the RNP stabilizer comprises poly-L-glutamic acid (PGA) or a suitable alternative. In certain embodiments, provided herein are compositions, methods, and/or kits comprising poly-L-glutamic acid.

The one or more RNP stabilizers can be present at any suitable concentration. In certain embodiments, the one or more RNP stabilizers are present at a concentration of at least 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, or 4.5 and/or not more than 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, or 5 μM per pmol RNP complex, for example 0.01-5 μM per pmol RNP complex, preferably 0.01-3 μM per pmol RNP complex, even more preferably 0.015-2.5 μM per pmol RNP complex, yet more preferably 0.01-1 μM per pmol RNP complex.

The one or more RNP stabilizers can be present at any suitable concentration. In certain embodiments where the one or more RNP stabilizers are a polymer product, the one or more RNP stabilizers are present at a concentration of at least 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, or 4.5 and/or not more than 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, or 5 μg μL⁻¹ per pmol RNP complex, for example 0.01-5 μg μL⁻¹ per pmol RNP complex, preferably 0.01-3 μg μL⁻¹ per pmol RNP complex, even more preferably 0.25-2.5 μg μL⁻¹ per pmol RNP complex, yet more preferably 0.5-1.5 μg μL⁻¹ per pmol RNP complex. In certain embodiments, the polymeric RNP stabilizer comprises PGA.

In certain embodiments, provided herein are compositions, methods, and/or kits comprising one or more additives that inhibit NHEJ, e.g., NHEJ inhibitor. In certain embodiments, the one or more additives that inhibit NHEJ are introduced to the target cell prior to delivery of the nucleic acid-guided nuclease, guide nucleic acid, and/or donor template, or one or more polynucleotides encoding the nucleic acid-guided nuclease, guide nucleic acid, and/or donor template. In certain embodiments, the one or more additives that inhibit NHEJ are introduced to the target cell after delivery of the nucleic acid-guided nuclease, guide nucleic acid, and/or donor template, or one or more polynucleotides encoding the nucleic acid-guided nuclease, guide nucleic acid, and/or donor template. In certain embodiments, the one or more additives that inhibit NHEJ are introduced to the target cell both prior to and after delivery of the nucleic acid-guided nuclease, guide nucleic acid, and/or donor template, or one or more polynucleotides encoding the nucleic acid-guided nuclease, guide nucleic acid, and/or donor template. In certain embodiments, the one or more additives that inhibit NHEJ are introduced into the cell medium, wherein the one or more NHEJ inhibitors can enter the cell.

In certain embodiments, the one or more additives that inhibit NHEJ comprise a molecule that indirectly or directly affects the interaction of p53-binding protein 1 (53BP1) with ubiquitylated histones at double stranded breaks, for example, iP53 or the like. In certain embodiments, the one or more additives that inhibit NHEJ comprise a molecule that directly or indirectly affects the interaction of Ku proteins with DNA, for example, STL127705 or the like. In certain embodiments, the one or more additives that inhibit NHEJ comprise a molecule that directly or indirectly affects the activity of DNA-dependent protein kinases, for example, M3814, KU-0060648, NU7026 or the like. In certain embodiments, the one or more additives that inhibit NHEJ comprise a molecule that directly or indirectly affects the activity of ATM-Rad3-related (ATR) proteins, for example VE-822 or the like. In certain embodiments, the one or more additives that inhibit NHEJ comprise a molecule that directly or indirectly affects the activity of ligases, e.g., ligase IV, for example SCR7 or the like. In certain embodiments, the one or more additives that inhibit NHEJ comprise a molecule that directly or indirectly affects the activity of RAD51 binding to ssDNA, for example RS-1 or the like. In certain embodiments, the one or more additives that inhibit NHEJ comprise a molecule that directly or indirectly affects the activity cell cycle stage progression, for example aphidicolin, mimosin, thymidine, hydroxy urea, nocodazole, ABT-751, XL413, or the like. In certain embodiments, the one or more additives that inhibit NHEJ comprise a molecule that directly or indirectly affects the activity beta-3-adrenergic receptors, for example L755507 or the like. In certain embodiments, the one or more additives that inhibit NHEJ comprise a molecule that directly or indirectly affects the activity of intracellular transport from endoplasmic reticulum (ER) to golgi, for example Brefeldin A or the like. In certain embodiments, the one or more additives that inhibit NHEJ comprise a molecule that directly or indirectly affects the activity histone deacetylases, for example valproic acid (VPA). In certain embodiments, the one or more additives that inhibit NHEJ comprise M3814.

In certain embodiments, the one or more NHEJ inhibitors are present at a concentration of at least 0.1, 0.25, 0.5, 0.75, 1, 1.25, 1.5, 1.75, 2, 2.5, 3, or 4 and/or not more than 0.25, 0.5, 0.75, 1, 1.25, 1.5, 1.75, 2, 2.5, 3, 4, or 5 μM, for example 0.1-5 μM, preferably 0.5-5 μM, even more preferably 1-3 μM, yet more preferably 2 μM. In certain embodiments, the one or more NHEJ inhibitors comprise M3814.

In certain embodiments, the NHEJ inhibitor reduces the activity of NHEJ-based repair, wherein the relative amount of repair via homology-directed repair (HDR) is increased. In certain embodiments, the amount of HDR compared to NHEJ is increased by at least 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.25, 2.5, 2.75, 3, 4, 5, 6, 7, 8, or 9-fold and/or not more than 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.25, 2.5, 2.75, 3, 4, 5, 6, 7, 8, 9, or 10-fold increased editing via homology directed repair (HDR) as compared to editing via NHEJ in cells treated with the one or more NHEJ inhibitors as compared to those not treated with one or more NHEJ inhibitors, for example 1.1-10-fold increased editing, preferably 1.1-5-fold increased editing, even more preferably 1.1-3-fold increased editing, yet more preferably 1.1-2-fold increased editing. In certain embodiments, the amount of INDEL formation due to NHEJ as measured by sequencing is reduced by at least 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.25, 2.5, 2.75, 3, 4, 5, 6, 7, 8, or 9-fold and/or not more than 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.25, 2.5, 2.75, 3, 4, 5, 6, 7, 8, 9, or 10-fold reduced INDEL formation due to NHEJ as compared to an untreated control, for example 1.1-10-fold reduced INDEL formation, preferably 1.1-5-fold reduced INDEL formation, even more preferably 1.1-3-fold reduced INDEL formation, yet more preferably 1.1-2-fold reduced INDEL formation. Any suitable sequencing method known in the art may be used to determine the relative types of edits generated following treatment.

In certain embodiments, provided herein are compositions, methods, and/or kits comprising nucleic acid-guided nucleases. In certain embodiments, provided herein are compositions, methods, and/or kits comprising engineered nucleic acid-guided nucleases. In certain embodiments, provided herein are compositions, methods, and/or kits wherein the nuclease comprises a Cas nuclease. In certain embodiments, provided herein are compositions, methods, and/or kits wherein the nuclease comprises a Class 1 or Class 2 Cas nuclease. In certain embodiments, provided herein are compositions, methods, and/or kits wherein the nuclease comprises a Type V nuclease. In certain embodiments, provided herein are compositions, methods, and/or kits wherein the nuclease comprises a Type V-A, V-B, V-C, V-D, or V-E nuclease. In certain embodiments, provided herein are compositions, methods, and/or kits wherein the nuclease comprises a Type V-A nuclease. In certain embodiments, provided herein are compositions, methods, and/or kits wherein the nuclease comprises a MAD, ABW, or ART nuclease. In certain embodiments, provided herein are compositions, methods, and/or kits wherein the nuclease comprises a MAD1, MAD2, MAD3, MAD4, MAD5, MAD6, MAD7, MAD8, MAD9, MAD10, MAD11, MAD12, MAD13, MAD14, MAD15, MAD16, MAD17, MAD18, MAD19, or MAD20 nuclease. In certain embodiments, provided herein are compositions, methods, and/or kits wherein the nuclease comprises an ART1, ART2, ART3, ART4, ART5, ART6, ART7, ART8, ART9, ART10, ART11, ART11*, ART12, ART13, ART14, ART15, ART16, ART17, ART18, ART19, ART20, ART21, ART22, ART23, ART24, ART25, ART26, ART27, ART28, ART29, ART30, ART31, ART32, ART33, ART34, or ART35 nuclease. In certain embodiments, provided herein are compositions, methods, and/or kits wherein the nuclease comprises a MAD2, MAD7, ART11, ART11*, or ART2 nuclease. In certain embodiments, provided herein are compositions, methods, and/or kits wherein the nuclease comprises one or more nuclear localization signals. In certain embodiments, provided herein are compositions, methods, and/or kits the nuclease comprises 1 or 4 nuclear localization signals, such as 1-4 NLS at the carboxy terminus, 1-4 NLS at the amino terminus, or a combination thereof. Additional nucleases and modifications thereof may be found in the Cas nuclease section below.

In certain embodiments, provided herein are compositions, methods, and/or kits wherein the relative amount (e.g., proportion) of gNA to nuclease results in improved editing efficiencies. In certain embodiments, provided herein are compositions, methods, and/or kits wherein the proportion of gNA to nuclease is at least 1, 1.05 1.1, 1.15, 1.2, 1.25, 1.3, 1.35, 1.4, 1.45, 1.5, 1.55, 1.6, 1.65, 1.7, 1.75, 1.8, 1.85, 1.9, or 1.95 and/or not more than 1.05 1.1, 1.15, 1.2, 1.25, 1.3, 1.35, 1.4, 1.45, 1.5, 1.55, 1.6, 1.65, 1.7, 1.75, 1.8, 1.85, 1.9, 1.95 or 2 parts for every part of nuclease, for example, 1-2 parts of gNA for every part of nuclease, preferably, 1.15-1.85 parts of gNA for every part of nuclease, even more preferably 1.25-1.75 parts of gNA for every part of nuclease, yet more preferably 1.5 parts of gNA for every part of nuclease. In certain embodiments, provided herein are compositions, methods, and/or kits the gNA and nuclease are present at 150:100 or 75:50 pmol respectively.

In certain embodiments, provided herein are compositions, methods, and/or kits wherein the amount of donor template delivered to the cell results affects editing efficiencies. In certain embodiments, provided herein are compositions, methods, and/or kits wherein the donor template is present at a concentration of at least 0.05, 0.01, 0.2, 0.3, 0.4, 0.5, 0.6, 0.7, 0.8, 0.9, 1, 1.25, 1.5, 1.75, 2, 3, or 4, and/or no more than 0.01, 0.2, 0.3, 0.4, 0.5, 0.6, 0.7, 0.8, 0.9, 1, 1.25, 1.5, 1.75, 2, 3, 4, or 5 μg μL⁻¹, for example 0.01-5 μg μL⁻¹, preferably 0.01-3 μg μL⁻¹, even more preferably 0.3-3 μg μL⁻¹, yet even more preferably 0.5-1.5 μg μL⁻¹.

In certain embodiments, provided herein are compositions comprising a nucleic acid-guided nuclease system and at least one additive that stabilizes the nucleic acid-guided nucleases. In certain embodiments, the nucleic acid-guided nuclease system comprises a naturally occurring system. In certain embodiments, the nucleic acid-guided nuclease system comprises an engineered, non-naturally occurring system. In certain embodiments, provided herein is a composition comprising one or more nucleases system comprising: a nucleic acid-guided nuclease; and a guide nucleic acid (gNA) compatible with and capable of binding to and activating the nucleic acid-guided nuclease, wherein the gNA comprises: a targeter nucleic acid comprising a targeter stem sequence and a spacer sequence, wherein the spacer sequence is complementary to a target nucleotide sequence within a target polynucleotide, for example a target polynucleotide of a genome of a human target cell; and a modulator nucleic acid comprising a modulator stem sequence complementary to the targeter stem sequence, and, optionally, a 5′ sequence; and at least one additive that stabilizes the nucleic acid-guided nuclease system. In certain embodiments, the composition comprises any nuclease disclosed herein in the Cas nuclease section. In certain embodiments, the composition comprises a single guide nucleic acid. In certain embodiments, the composition comprises a dual guide nucleic acid as disclosed herein in the Guide nucleic acids section. In certain embodiments, the composition comprises a guide nucleic acid comprising a spacer sequence comprising any one of SEQ ID NOs: 86-384 as shown in Table 5. In certain embodiments, the guide nucleic acid comprises one or more chemical modifications as disclosed herein in the gNA modifications section. In certain embodiments, the composition further comprises a donor template as disclosed herein in the Donor templates section. In certain embodiments, the composition is introduced into one or more cells, wherein the composition can bind to a target sequence within a target polynucleotide within the genome of a human target cell and generate a strand break in at least one strand at or near the target sequence. In certain embodiments, the NHEJ inhibitor is added to the one or more human target cells prior to or after delivery of the composition. In certain embodiments, at least a portion of the donor template is introduced into the target polynucleotide at or near the strand break via an innate cell repair mechanism. In certain embodiments the innate repair mechanism comprises homology directed repair (HDR), e.g., homologous recombination.

In certain embodiments, provided herein are compositions comprising one or more human target cells comprising at least one additive that reduces non-homologous end joining (NHEJ). In certain embodiments, provided herein are compositions further comprising a nucleic acid-guided nuclease as disclosed herein in Cas nuclease section. In certain embodiments, provided herein is a composition comprising: a nucleic acid-guided nuclease capable of binding to a compatible guide nucleic acid (gNA) comprising a spacer sequence complementary to a target nucleotide sequence within a target polynucleotide, e.g., a target polynucleotide of a genome of a human target cell and generating a strand break in one or both strands of the target polynucleotide; one or more human target cells; and at least one additive that reduces non-homologous end joining (NHEJ)-based DNA repair. In certain embodiments provided herein is a composition comprising a human cell comprising: a nuclease capable of binding to a compatible guide nucleic acid (gNA) comprising a spacer sequence complementary to a target nucleotide sequence within a target polynucleotide of a genome of the human cell and generating a strand break in one or both strands of the target polynucleotide; and at least one additive that reduces non-homologous end joining (NHEJ)-based DNA repair. In certain embodiments, the composition further comprises a guide nucleic acid as disclosed herein in the Guide nucleic acids section. In certain embodiments, the composition comprises a guide nucleic acid comprising a spacer sequence comprising any one of SEQ ID NOs: 86-384 as shown in Table 5. In certain embodiments, the guide nucleic acid comprises one or more chemical modifications as disclosed herein in the gNA modifications section. In certain embodiments, the nuclease forms a nucleic acid-guided nuclease complex with the guide nucleic acid. In certain embodiments, the composition further comprises a donor template as disclosed herein in the Donor templates section. In certain embodiments, the nuclease complex can bind to a target sequence within a target polynucleotide within the genome of a human target cell and generate a strand break in at least one strand at or near the target sequence. In certain embodiments, the NHEJ inhibitor is added to the one or more human target cells prior to or after delivery of the composition. In certain embodiments, at least a portion of the donor template is introduced into the target polynucleotide at or near the strand break via an innate cell repair mechanism. In certain embodiments the innate repair mechanism comprises homology directed repair (HDR), e.g., homologous recombination.

In certain embodiments, provided herein are methods. In certain embodiments, provided herein are methods for engineering cells. In certain embodiments, provided herein are methods for engineering human cells. In certain embodiments, provided herein are methods for efficiently engineering human cells. In certain embodiments, provided herein is a method for editing a target polynucleotide in the genome of a human target cell comprising one or more of steps (A) to (G), wherein step (A) comprises forming the nuclease complex by combining one or more nucleases with one or more guide nucleic acids and/or one or more RNP stabilizers; step (B) comprises delivering the nuclease system to the human target cell; step (C) comprises delivering one or more donor templates to the human target cell; step (D) comprises contacting the target polynucleotide with a nuclease system comprising: a nucleic acid-guided nuclease; and a guide nucleic acid (gNA) compatible with and capable of binding to and activating the nucleic acid-guided nuclease, wherein the gNA comprises: a targeter nucleic acid comprising a targeter stem sequence and a spacer sequence, wherein the spacer sequence is complementary to a target nucleotide sequence within a target polynucleotide, for example a target polynucleotide of a genome of a human target cell; and a modulator nucleic acid comprising a modulator stem sequence complementary to the targeter stem sequence, and, optionally, a 5′ sequence; step (E) comprises contacting the cell with at least one additive that reduces non-homologous end joining (NHEJ)-based DNA repair; step (F) comprises growing the cell in a suitable growth medium; step (G) isolating one or more cells that demonstrate the genotype and/or phenotype of interest. In certain embodiments, any number of steps (A) through (G) may be performed in any order. In certain embodiments, the one or more steps (A) through (G) may be performed on the same population of cells. In certain embodiments, the one or more steps (A) through (G) may be performed on the progeny of a first set of cells treated with the one or more steps (A) through (G).

In certain embodiments, the method comprises the following steps and order: step (A) is performed wherein the gNA is combined with the RNP stabilizer prior to addition of the nuclease to form a stabilized nucleic acid-guided nuclease complex; step (B) and step (C) are performed sequentially such that the one or more nucleic acid-guided nuclease complexes are combined with the one or more donor templates and delivered to the one or more human target cells; step (D); step (E) wherein the one or more NHEJ inhibitors are added to the cell recovery medium; step (F).

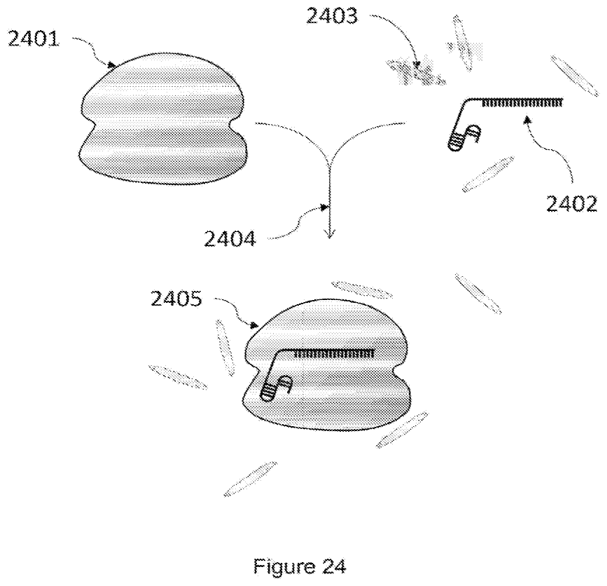

Step (A) is illustrated in FIG. 24. FIG. 24 shows the combination of a guide nucleic acid (2402) with one or more RNP stabilizers (2403). The nuclease (2401) is combined (2404) with the gNA-RNP stabilizer mixture, whereby a stabilized nucleic acid-guided nuclease complex (2405) is formed. The gNA molecule can comprise either a single or dual guide nucleic acid. A single gNA is shown in FIG. 24 for illustrative purposes only.

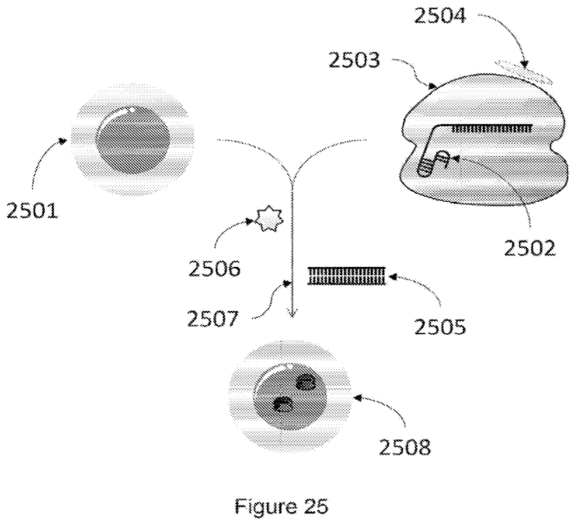

Steps (B) through (E) are illustrated in FIG. 25. FIG. 25 shows the delivery (2507) of the stabilized RNP complex (2503) comprising a nuclease, one or more RNP stabilizer (2504), and a guide nucleic acid (2502) along with, optionally, one or more donor templates (2505) to one or more human target cells (2501), resulting in a cell comprising a one or more nuclease complex and/or one or more donor templates (2508). The one or more NHEJ inhibitors (2506) may be added before or after delivery of the nucleic acid-guided nuclease complex and/or the one or more donor templates.

In certain embodiments, the human cell comprises an immune cell or a stem cell. In certain embodiments, the immune cell comprises a neutrophil, eosinophil, basophil, mast cell, monocyte, macrophage, dendritic cell, natural killer cell, or a lymphocyte. In certain embodiments, the immune cell comprises a T cell. In certain embodiments, the T cell comprises a CAR-T cell. In certain embodiments, the stem cell comprises a human pluripotent, multipotent stem cell, embryonic stem cell, induced pluripotent stem cell, CD34+ stem cell, or hematopoietic stem cell. In certain embodiments, the human cell is allogeneic, I,e, a cell that provokes little or no immune response when introduced into an allogeneic host and produces little or no graft versus host response.

### II. ENGINEERED NON-NATURALLY-OCCURRING DUAL GUIDE CRISPR-CAS SYSTEMS

A CRISPR-Cas system generally comprises a Cas protein and one or more guide nucleic acids (gNAs). The Cas protein can be directed to a specific location in a double-stranded DNA target by recognizing a protospacer adjacent motif (PAM) in the non-target strand of the DNA, and the one or more guide nucleic acids can be directed to a specific location by hybridizing with a target nucleotide sequence, also referred to herein as a target sequence, in the target strand of the target polynucleotide. Typically, both PAM recognition and target nucleotide sequence hybridization are required for stable binding of a CRISPR-Cas complex to the DNA target and, if the Cas protein has an effector function (e.g., nuclease activity), activation of the effector function. As a result, when creating a CRISPR-Cas system, a guide nucleic acid can be designed to comprise a nucleotide sequence called a spacer sequence that is at least partially complementary to and can hybridize with a target nucleotide sequence, where target nucleotide sequence is located adjacent to a PAM in an orientation operable with the Cas protein. It has been observed that not all CRISPR-Cas systems designed by these criteria are equally effective. The larger polynucleotide in which a target nucleotide sequence is located may be referred to as a target polynucleotide; e.g., a chromosome or other genomic DNA, or portion thereof, or any other suitable polynucleotide within which a target nucleotide sequence is located. The target polynucleotide in double stranded DNA comprises two strands. The strand of the DNA duplex to which the spacer sequence is complementary herein is called the “target strand,” while the strand to which the spacer sequence shares sequence identity herein is called the “non-target strand.”

Two distinct classes of CRISPR-Cas systems have been identified. Class 1 CRISPR-Cas systems utilize multi-protein effector complexes, whereas class 2 CRISPR-Cas systems utilize single-protein effectors (see, Makarova et al. (2017) CELL, 168:328). Among the types of class 2 CRISPR-Cas systems, type II and type V systems typically target DNA and type VI systems typically target RNA (id.). Naturally occurring type II effector complexes include Cas9, CRISPR RNA (crRNA), and trans-activating CRISPR RNA (tracrRNA), but the crRNA and tracrRNA can be fused as a single guide RNA in an engineered system for simplicity (see, Wang et al. (2016) ANNU. REV. BIOCHEM., 85:227). Certain naturally occurring type V systems, such as type V-A, type V-C, and type V-D systems, do not require tracrRNA and use crRNA alone as the guide for cleavage of target DNA (see, Zetsche et al. (2015) CELL, 163:759; Makarova et al. (2017) CELL, 168:328.

Naturally occurring type II CRISPR-Cas systems (e.g., CRISPR-Cas9 systems) generally comprise two guide nucleic acids, called crRNA and tracrRNA, which form a complex by nucleotide hybridization. Single guide nucleic acids capable of activating type II Cas nucleases have been developed, for example, by linking the crRNA and the tracrRNA (see, e.g., U.S. Pat. Nos. 10,266,850 and 8,906,616). Naturally occurring type II Cas proteins comprise a RuvC-like nuclease domain and an HNH endonuclease domain, and recognize a 3′ G-rich PAM located immediately downstream from the target nucleotide sequence, the orientation determined using the non-target strand (i.e., the strand not hybridized with the spacer sequence) as the coordinate. The CRISPR-Cas systems cleave a double-stranded DNA to generate a blunt end. The cleavage site is generally 3-4 nucleotides upstream from the PAM on the non-target strand.

Naturally occurring Type V-A, Type V-C, and Type V-D CRISPR-Cas systems lack a tracrRNA and rely on a single crRNA to guide the CRISPR-Cas complex to the target polynucleotide. Dual guide nucleic acids capable of activating type V-A, type V-C, or type V-D Cas nucleases have been developed, for example, by splitting the single crRNA into a targeter nucleic acid and a modulator nucleic acid (see, e.g., International (PCT) Application Publication No. WO 2021/067788). Naturally occurring type V-A Cas proteins comprise a RuvC-like nuclease domain but lack an HNH endonuclease domain, and recognize a 5′ T-rich PAM located immediately upstream from the target nucleotide sequence, the orientation determined using the non-target strand (i.e., the strand not hybridized with the spacer sequence) as the coordinate. These CRISPR-Cas systems cleave a double-stranded DNA to generate a staggered double-stranded break rather than a blunt end. The cleavage site is distant from the PAM site (e.g., separated by at least 10, 11, 12, 13, 14, or 15 nucleotides downstream from the PAM on the non-target strand and/or separated by at least 15, 16, 17, 18, or 19 nucleotides upstream from the sequence complementary to PAM on the target strand).

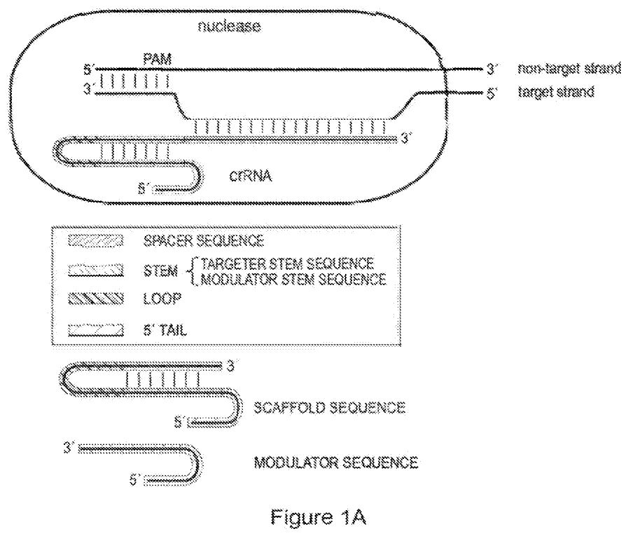

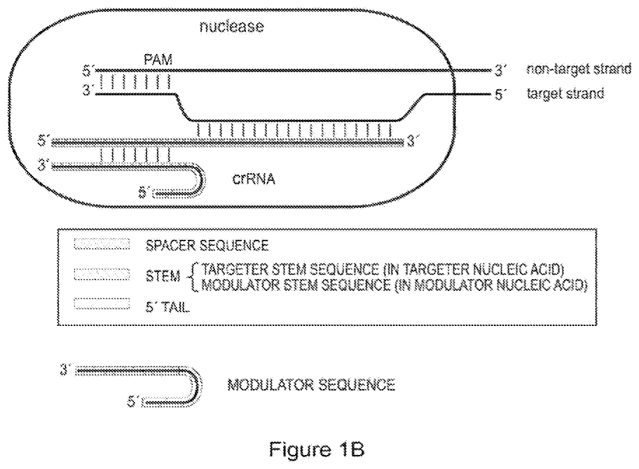

Elements in an exemplary single guide CRISPR Cas system, e.g., a type V-A CRISPR-Cas system, are shown in FIG. 1A. The single gNA can also be called a “crRNA” or “single gRNA” where it is present in the form of an RNA. It can comprise, from 5′ to 3′, an optional 5′ sequence, e.g., a tail, a modulator stem sequence, a loop, a targeter stem sequence complementary to the modulator stem sequence, and a spacer sequence that is at least partially complementary to and can hybridize with a target sequence in the target strand of the target polynucleotide. Where a 5′ tail is present, the sequence including the 5′ tail and the modulator stem sequence can also be called a “modulator sequence” herein. A fragment of the single guide nucleic acid from the optional 5′ tail to the targeter stem sequence, also called a “scaffold sequence” herein, bind the Cas protein. In addition, the PAM in the non-target strand of the target DNA binds the Cas protein.

Elements in an exemplary dual guide type CRISPR Cas system, e.g., a dual guide type V-A CRISPR-Cas system are shown in FIG. 1B. The first guide nucleic acid, which can be called a “modulator nucleic acid” herein, comprises, from 5′ to 3′, an optional 5′ tail and a modulator stem sequence. Where a 5′ tail is present, the sequence including the 5′ tail and the modulator stem sequence can also called a “modulator sequence” herein. The second guide nucleic acid, which can be called “targeter nucleic acid” herein, comprises, from 5′ to 3′, a targeter stem sequence complementary to the modulator stem sequence and a spacer sequence that is at least partially complementary to and can hybridize with the target sequence in the target strand of the target polynucleotide. The duplex between the modulator stem sequence and the targeter stem sequence, plus the optional 5′ tail, constitute a structure that binds the Cas protein. In addition, the PAM in the non-target strand of the target DNA binds the Cas protein. It is understood that, in a dual gNA, e.g., dual gRNA, the targeter nucleic acid and the modulator nucleic acid, while not in the same nucleic acids, i.e., not linked end-to-end through a traditional internucleotide bond, can be covalently conjugated to each other through one or more chemical modifications introduced into these nucleic acids, thereby increasing the stability of the double-stranded complex and/or improving other characteristics of the system.

The terms “targeter stem sequence” and “modulator stem sequence,” as used herein, can refer to a pair of nucleotide sequences in one or more guide nucleic acids that hybridize with each other. When a targeter stem sequence and a modulator stem sequence are contained in a single guide nucleic acid, the targeter stem sequence is proximal to a spacer sequence designed to hybridize with a target nucleotide sequence, and the modulator stem sequence is proximal to the targeter stem sequence. When a targeter stem sequence and a modulator stem sequence are in separate nucleic acids, the targeter stem sequence is in the same nucleic acid as a spacer sequence designed to hybridize with a target nucleotide sequence. In a CRISPR-Cas system that naturally includes separate crRNA and tracrRNA (e.g., a type II system), the duplex formed between the targeter stem sequence and the modulator stem sequence corresponds to the duplex formed between the crRNA and the tracrRNA. In a CRISPR-Cas system that naturally includes a single crRNA but no tracrRNA (e.g., a type V-A system), the duplex formed between the targeter stem sequence and the modulator stem sequence corresponds to the stem portion of a stem-loop structure in the scaffold sequence of the crRNA. It is understood that 100% complementarity is not required between the targeter stem sequence and the modulator stem sequence. In a type V-A CRISPR-Cas system, however, the targeter stem sequence is typically 100% complementary to the modulator stem sequence.

### A. Cas Proteins

A guide nucleic acid, either as a single guide nucleic acid alone (targeter and modulator nucleic acids are part of a single polynucleotide) or as a dual gNA comprising separate targeter nucleic acid used in combination with a cognate modulator nucleic acid, is capable of binding a CRISPR Associated (Cas) protein, e.g., a Cas nuclease. In certain embodiments, the guide nucleic acid, either as a single guide nucleic acid alone (targeter and modulator nucleic acids are part of a single polynucleotide) or as a dual gNA comprising separate targeter nucleic acid used in combination with a cognate modulator nucleic acid, is capable of activating a Cas nuclease. A gNA capable of activating a particular Cas nuclease is said to be “compatible” with the Cas nuclease; a Cas nuclease capable of being activated by a particular gNA is said to be “compatible” with the gNA.

The terms “CRISPR-Associated protein,” “Cas protein,” and “Cas,” as used interchangeably herein, can refer to a naturally occurring Cas protein or an engineered Cas protein. Non-limiting examples of Cas protein engineering include but are not limited to mutations and modifications of the Cas protein that alter the activity of the Cas, alter the PAM specificity, broaden the range of recognized PAMs, and/or reduce the ability to modify one or more off-target loci as compared to a corresponding unmodified Cas. In certain embodiments, the altered activity of engineered Cas comprises altered ability (e.g., specificity or kinetics) to bind a naturally occurring gNA, e.g., gRNA or engineered gNA, e.g., gRNA, altered ability (e.g., specificity or kinetics) to bind a target nucleotide sequence, altered processivity of nucleic acid scanning, and/or altered effector (e.g., nuclease) activity. A Cas protein having nuclease activity can be referred to as a “CRISPR-Associated nuclease” or “Cas nuclease,” or simply “nuclease,” as used interchangeably herein.

In certain embodiments, the Cas protein is a type V-A, type V-C, or type V-D Cas protein. In certain embodiments, the Cas protein is a type V-A Cas protein. In other embodiments, the Cas protein is a type II Cas protein, e.g., a Cas9 protein.

In certain embodiments, a type V-A Cas nucleases comprises Cpf1. Cpf1 proteins are known in the art and are described, e.g., in U.S. Pat. Nos. 9,790,490 and 10,113,179. Cpf1 orthologs can be found in various bacterial and archaeal genomes. For example, in certain embodiments, the Cpf1 protein is derived from Francisella novicida U112 (Fn), Acidaminococcus sp. BV3L6 (As), Lachnospiraceae bacterium ND2006 (Lb), Lachnospiraceae bacterium MA2020 (Lb2), Candidatus Methanoplasma termitum (CMt), Moraxella bovoculi 237 (Mb), Porphyromonas crevioricanis (Pc), Prevotella disiens (Pd), Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2 33 10, Parcubacteria bacterium GW2011_GWC2_44_17, Smithella sp. SCADC, Eubacterium eligens, Leptospira inadai, Porphyromonas macacae, Prevotella bryantii, Proteocatella sphenisci, Anaerovibrio sp. RM50, Moraxella caprae, Lachnospiraceae bacterium COE1, or Eubacterium coprostanoligenes.

In certain embodiments, a type V-A Cas nuclease comprises AsCpf1 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 3 of International (PCT) Application Publication No. WO 2021/158918. In certain embodiments, a type V-A Cas protein comprises the amino acid sequence set forth in SEQ ID NO: 3 of International (PCT) Application Publication No. WO 2021/158918.

In certain embodiments, a type V-A Cas nuclease comprises LbCpf1 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 4 of International (PCT) Application Publication No. WO 2021158918. In certain embodiments, a type V-A Cas protein comprises the amino acid sequence set forth in SEQ ID NO: 4 of International (PCT) Application Publication No. WO 2021/158918.

In certain embodiments, a type V-A Cas nuclease comprises FnCpf1 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 5 of International (PCT) Application Publication No. WO 2021158918. In certain embodiments, a type V-A Cas protein comprises the amino acid sequence set forth in SEQ ID NO: 5 of International (PCT) Application Publication No. WO 2021/158918.

In certain embodiments, a type V-A Cas nuclease comprises Prevotella bryantii Cpf1 (PbCpf1) or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 6 of International (PCT) Application Publication No. WO 2021/158918. In certain embodiments, a type V-A Cas protein comprises the amino acid sequence set forth in SEQ ID NO: 6 of International (PCT) Application Publication No. WO 2021/158918.

In certain embodiments, a type V-A Cas nuclease comprises Proteocatella sphenisci Cpf1 (PsCpf1) or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 7 of International (PCT) Application Publication No. WO 2021158918. In certain embodiments, a type V-A Cas protein comprises the amino acid sequence set forth in SEQ ID NO: 7 of International (PCT) Application Publication No. WO 2021/158918.

In certain embodiments, a type V-A Cas nuclease comprises Anaerovibrio sp. RM50 Cpf1 (As2Cpf1) or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 8 of International (PCT) Application Publication No. WO 2021158918. In certain embodiments, a type V-A Cas protein comprises the amino acid sequence set forth in SEQ ID NO: 8 of International (PCT) Application Publication No. WO 2021/158918.

In certain embodiments, a type V-A Cas nuclease comprises Moraxella caprae Cpf1 (McCpf1) or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 9 of International (PCT) Application Publication No. WO 2021/158918. In certain embodiments, a type V-A Cas protein comprises the amino acid sequence set forth in SEQ ID NO: 9 of International (PCT) Application Publication No. WO 2021/158918.

In certain embodiments, a type V-A Cas nuclease comprises Lachnospiraceae bacterium COE1 Cpf1 (Lb3Cpf1) or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 10 of International (PCT) Application Publication No. WO 2021158918. In certain embodiments, a type V-A Cas protein comprises the amino acid sequence set forth in SEQ ID NO: 10 of International (PCT) Application Publication No. WO 2021/158918.

In certain embodiments, a type V-A Cas nuclease comprises Eubacterium coprostanoligenes Cpf1 (EcCpf1) or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 11 of International (PCT) Application Publication No. WO 2021158918. In certain embodiments, a type V-A Cas protein comprises the amino acid sequence set forth in SEQ ID NO: 11 of International (PCT) Application Publication No. WO 2021/158918.

In certain embodiments, a type V-A Cas nuclease is not Cpf1. In certain embodiments, a type V-A Cas nuclease is not AsCpf1.

In certain embodiments, a type V-A Cas nuclease comprises MAD1, MAD2, MAD3, MAD4, MAD5, MAD6, MAD7, MAD8, MAD9, MAD10, MAD11, MAD12, MAD13, MAD14, MAD15, MAD16, MAD17, MAD18, MAD19, or MAD20, or variants thereof. MAD1-MAD20 are known in the art and are described in U.S. Pat. No. 9,982,279.

In certain embodiments, a type V-A Cas nuclease comprises MAD7 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 37. In certain embodiments, a type V-A Cas protein comprises the amino acid sequence set forth in SEQ ID NO: 37.

MAD7
(SEQ ID NO: 37)
MNNGTNNFQNFIGISSLQKTLRNALIPTETTQQFIVKNGIIKEDELRGENRQILKDIMDDYYRGF
ISETLSSIDDIDWTSLFEKMEIQLKNGDNKDTLIKEQTEYRKAIHKKFANDDRFKNMFSAKLISD
ILPEFVIHNNNYSASEKEEKTQVIKLESRFATSFKDYFKNRANCESADDISSSSCHRIVNDNAEI
FFSNALVYRRIVKSLSNDDINKISGDMKDSLKEMSLEEIYSYEKYGEFITQEGISFYNDICGKVN
SEMNLYCQKNKENKNLYKLQKLHKQILCIADTSYEVPYKFESDEEVYQSVNGELDNISSKHIVER
LRKIGDNYNGYNLDKIYIVSKFYESVSQKTYRDWETINTALEIHYNNILPGNGKSKADKVKKAVK
NDLQKSITEINELVSNYKLCSDDNIKAETYIHEISHILNNFEAQELKYNPEIHLVESELKASELK
NVLDVIMNAFHWCSVFMTEELVDKDNNFYAELEEIYDEIYPVISLYNLVRNYVTQKPYSTKKIKL
NFGIPTLADGWSKSKEYSNNAIILMRDNLYYLGIFNAKNKPDKKIIEGNTSENKGDYKKMIYNLL
PGPNKMIPKVFLSSKTGVETYKPSAYILEGYKQNKHIKSSKDEDITFCHDLIDYFKNCIAIHPEW
KNFGFDFSDTSTYEDISGFYREVELQGYKIDWTYISEKDIDLLQEKGQLYLFQIYNKDFSKKSTG
NDNLHTMYLKNLFSEENLKDIVLKLNGEAEIFFRKSSIKNPIIHKKGSILVNRTYEAEEKDQFGN
IQIVRKNIPENIYQELYKYFNDKSDKELSDEAAKLKNVVGHHEAATNIVKDYRYTYDKYFLHMPI
TINFKANKTGFINDRILQYIAKEKDLHVIGIDRGERNLIYVSVIDTCGNIVEQKSFNIVNGYDYQ
IKLKQQEGARQIARKEWKEIGKIKEIKEGYLSLVIHEISKMVIKYNAIIAMEDLSYGFKKGRFKV
ERQVYQKFETMLINKLNYLVFKDISITENGGLLKGYQLTYIPDKLKNVGHQCGCIFYVPAAYTSK
IDPTTGFVNIFKFKDLTVDAKREFIKKFDSIRYDSEKNLFCFTEDYNNFITQNTVMSKSSWSVYT
YGVRIKRRFVNGRFSNESDTIDITKDMEKTLEMTDINWRDGHDLRQDIIDYEIVQHIFEIFRLTV
QMRNSLSELEDRDYDRLISPVLNENNIFYDSAKAGDALPKDADANGAYCIALKGLYEIKQITENW
KEDGKFSRDKLKISNKDWFDFIQNKRYL

In certain embodiments, a type V-A Cas nuclease comprises MAD2 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 38. In certain embodiments, a type V-A Cas protein comprises the amino acid sequence set forth in SEQ ID NO: 38.

MAD2
(SEQ ID NO: 38)
MSSLTKFTNKYSKQLTIKNELIPVGKTLENIKENGLIDGDEQLNENYQKAKIIVDDELRDFINKA
LNNTQIGNWRELADALNKEDEDNIEKLQDKIRGIIVSKFETFDLFSSYSIKKDEKIIDDDNDVEE
EELDLGKKTSSFKYIFKKNLFKLVLPSYLKTTNQDKLKIISSFDNFSTYFRGFFENRKNIFTKKP
ISTSIAYRIVHDNFPKFLDNIRCFNVWQTECPQLIVKADNYLKSKNVIAKDKSLANYFTVGAYDY
FLSQNGIDFYNNIIGGLPAFAGHEKIQGLNEFINQECQKDSELKSKLKNRHAFKMAVLFKQILSD
REKSFVIDEFESDAQVIDAVKNFYAEQCKDNNVIFNLLNLIKNIAFLSDDELDGIFIEGKYLSSV
SQKLYSDWSKLRNDIEDSANSKQGNKELAKKIKINKGDVEKAISKYEFSLSELNSIVHDNTKESD
LLSCTLHKVASEKLVKVNEGDWPKHLKNNEEKQKIKEPLDALLEIYNTLLIFNCKSFNKNGNFYV
DYDRCINELSSVVYLYNKTRNYCTKKPYNTDKFKLNFNSPQLGEGFSKSKENDCLTLLFKKDDNY
YVGIIRKGAKINFDDTQAIADNTDNCIFKMNYFLLKDAKKFIPKCSIQLKEVKAHFKKSEDDYIL
SDKEKFASPLVIKKSTFLLATAHVKGKKGNIKKFQKEYSKENPTEYRNSLNEWIAFCKEFLKTYK
AATIFDITTLKKAEEYADIVEFYKDVDNLCYKLEFCPIKTSFIENLIDNGDLYLFRINNKDFSSK
STGTKNLHTLYLQAIFDERNLNNPTIMLNGGAELFYRKESIEQKNRITHKAGSILVNKVCKDGTS
LDDKIRNEIYQYENKFIDTLSDEAKKVLPNVIKKEATHDITKDKRFTSDKFFFHCPLTINYKEGD
TKQFNNEVLSFLRGNPDINIIGIDRGERNLIYVTVINQKGEILDSVSENTVINKSSKIEQTVDYE
EKLAVREKERIEAKRSWDSISKIATLKEGYLSAIVHEICLLMIKHNAIVVLENLNAGFKRIRGGL
SEKSVYQKFEKMLINKLNYFVSKKESDWNKPSGLINGLQLSDQFESFEKLGIQSGFIFYVPAAYT
SKIDPTTGFANVLNLSKVRNVDAIKSFFSNFNEISYSKKEALFKFSFDLDSLSKKGFSSFVKESK
SKWNVYTFGERIIKPKNKQGYREDKRINLTFEMKKLLNEYKVSFDLENNLIPNLTSANLKDTFWK
ELFFIFKTTLQLRNSVTNGKEDVLISPVKNAKGEFFVSGTHNKTLPQDCDANGAYHIALKGLMIL
ERNNLVREEKDTKKIMAISNVDWFEYVQKRRGVL

In certain embodiments, a type V-A Cas nucleases comprises Csm1. Csm1 proteins are known in the art and are described in U.S. Pat. No. 9,896,696. Csm1 orthologs can be found in various bacterial and archaeal genomes. For example, in certain embodiments, a Csm1 protein is derived from Smithella sp. SCADC (Sm), Sulfuricurvum sp. (Ss), or Microgenomates (Roizmanbacteria) bacterium (Mb).

In certain embodiments, a type V-A Cas nuclease comprises SmCsm1 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 12 of International (PCT) Application Publication No. WO 2021/158918. In certain embodiments, a type V-A Cas protein comprises the amino acid sequence set forth in SEQ ID NO: 12 of International (PCT) Application Publication No. WO 2021/158918.

In certain embodiments, a type V-A Cas nuclease comprises SsCsm1 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 13 of International (PCT) Application Publication No. WO 2021/158918. In certain embodiments, a type V-A Cas protein comprises the amino acid sequence set forth in SEQ ID NO: 13 of International (PCT) Application Publication No. WO 2021/158918.

In certain embodiments, a type V-A Cas nuclease comprises MbCsm1 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 14 of International (PCT) Application Publication No. WO 2021/158918. In certain embodiments, a type V-A Cas protein comprises the amino acid sequence set forth in SEQ ID NO: 14 of International (PCT) Application Publication No. WO 2021/158918.

In certain embodiments, the type V-A Cas nuclease comprises an ART nuclease or a variant thereof. In general, such nucleases sequences have <60% AA sequence similarity to Cas12a, <60% AA sequence similarity to a positive control nuclease, and >80% query cover. In certain embodiments, the Type V-A nuclease comprises an ART1, ART2, ART3, ART4, ART5, ART6, ART7, ART8, ART9, ART10, ART11, ART12, ART13, ART14, ART15, ART16, ART17, ART18, ART19, ART20, ART21, ART22, ART23, ART24, ART25, ART26, ART27, ART28, ART28, ART30, ART31, ART32, ART33, ART34, ART35, or ART11* (i.e., ART11_L679F, i.e., ART11 wherein leucine (L) at amino acid position 679 is replaced with phenylalanine (F)) nuclease, as shown in Table 1. In certain embodiments, the type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence designated for the individual ART nuclease as shown in Table 1. In certain embodiments, provided is a nucleic acid-guided nuclease comprising a nucleic acid-guided nuclease polypeptide having at least 85% identity to an amino acid sequence represented by SEQ ID NOs: 1-36 or a nucleic acid encoding a nucleic acid-guided nuclease polypeptide comprising at least 85% identity with the polynucleotide represented by SEQ ID NOs: 1-36. In certain embodiments, provided is a nucleic acid-guided nuclease comprising a polypeptide having at least 90% identity to the amino acid sequence represented by SEQ ID NOs: 1-36, wherein the polypeptide does not contain a peptide motif of YLFQIYNKDF (SEQ ID NO: 39). In certain embodiments, provided is a nucleic acid-guided nuclease comprising a nucleic acid encoding a polypeptide having at least 90% identity to nucleic acids represented by SEQ ID NOs: 808-845 wherein an encoded polypeptide does not contain a peptide motif of YLFQIYNKDF (SEQ ID NO: 39). In certain embodiments, provided is a nucleic acid-guided nuclease wherein the polypeptide comprises at least 90% identity with the amino acid sequence represented by SEQ ID NOs: 1-9. In certain embodiments, provided is a nucleic acid-guided nuclease, wherein the polypeptide comprises a polypeptide comprising at least 90% identity with the amino acid sequence represented by SEQ ID NO: 2, 11, or 36.

TABLE 1
ART nucleases

| | SEQ | |
|---|---|---|
| Name | ID NO | Amino Acid Sequence |
| ART1 | 1 | METFSGFTNLYPLSKTLRFRLIPVGETLKHFIDSGILEEDQHRAESYVK |
| | | VKAIIDDYHRAYIENSLSGFELPLESTKFNSLEEYYLYHNIRNKTEEIQ |
| | | NLSSKVRTNLRKQVVAQLTKNEIFKRIDKKELIQSDLIDFVKNEPDANE |
| | | KIALISEFRNFTVYFKGFHENRRNMYSDEEKSTSIAFRLIHENLPKFID |
| | | NMEVFAKIQNTSISENFDAIQKELCPELVTLCEMFKLGYFNKTLSQKQI |
| | | DAYNTVIGGKTTSEGKKIKGLNEYINLYNQQHKQEKLPKMKLLFKQILS |
| | | DRESASWLPEKFENDSQVVGAIVNFWNTIHDTVLAEGGLKTIIASLGSY |
| | | GLEGIFLKNDLQLTDISQKATGSWGKISSEIKQKIEVMNPQKKKESYET |
| | | YQERIDKIFKSYKSFSLAFINECLRGEYKIEDYFLKLGAVNSSSLQKEN |
| | | HFSHILNTYTDVKEVIGLYSESTDTKLIQDNDSIQKIKQFLDAVKDLQA |
| | | YVKPLLGNGDETGKDERFYGDLIEYWSLLDLITPLYNMVRNYVTQKPYS |
| | | VDKIKINFQNPTLLNGWDLNKETDNTSVILRRDGKYYLAIMNNKSRKVF |
| | | LKYPSGTDRNCYEKMEYKLLPGANKMLPKVFFSKSRINEFMPNERLLSN |
| | | YEKGTHKKSGTCFSLDDCHTLIDFFKKSLDKHEDWKNFGFKFSDTSTYE |
| | | DMSGFYKEVENQGYKLSFKPIDATYVDQLVDEGKIFLFQIYNKDFSEHS |
| | | KGTPNMHTLYWKMLFDETNLGDVVYKLNGEAEVFFRKASINVSHPTHPA |
| | | NIPIKKKNLKHKDEERILKYDLIKDKRYTVDQFQFHVPITMNFKADGNG |
| | | NINQKAIDYLRSASDTHIIGIDRGERNLLYLVVIDGNGKICEQFSLNEI |
| | | EVEYNGEKYSTNYHDLLNVKENERKQARQSWQSIANIKDLKEGYLSQVI |
| | | HKISELMVKYNAIVVLEDLNAGFMRGRQKVEKQVYQKFEKKLIEKLNYL |
| | | VFKKQSSDLPGGLMHAYQLANKFESFNTLGKQSGFLFYIPAWNTSKMDP |
| | | VTGFVNLFDVKYESVDKAKSFFSKFDSIRYNVERDMFEWKFNYGEFTKK |
| | | AEGTKTDWTVCSYGNRIITFRNPDKNSQWDNKEINLTENIKLLFERFGI |
| | | DLSSNLKDEIMQRTEKEFFIELISLFKLVLQMRNSWTGTDIDYLVSPVC |
| | | NENGEFFDSRNVDETLPQNADANGAYNIARKGMILLDKIKKSNGEKKLA |
| | | LSITNRE WLSFAQGCCKNG |
| ART2 | 2 | MLSNFTNQYQLSKTLRFELKPVGDTLKHIEKSGLIAQDEIRSQEYQEVK |
| | | TIIDKYHKAFIDEALQNVVLSNLEEYEALFFERNRDEKAFEKLQAVLRK |
| | | EIVAHFKQHPQYKTLFKKELIKADLKNWQELSDAEKELVSHFDNFTTYF |
| | | TGFHENRANMYTDEAKHSSIAYRIIHENLPIFLTNKKLFETIKQKAPHL |
| | | AQETQDALLEYLSGAIVEDMFELSYFNHLLSQTHIDLYNQMIGGVKQDS |
| | | LKIQGLNEKINLYRQANGLSKRELPNLKPLHKQILSDRETLSWLPESFE |
| | | SDEELMQGVQAYFESEVLAFECCDGKVNLLEKLPELLHQTQDYDFSKVY |
| | | FKNDLALTAASQAIFKDYRIIKEALWEVNKPKKSKDLVADEEKFFNKKN |
| | | SYFSIEQIDGALNSAQLSANMMHYFQSESTKVIEQIQLTYNDWKRNSSN |
| | | KELLKAFLDALLSYQRLLKPLNAPNDLEKDVAFYAYFDAYFTSLCGVVK |
| | | LYDKVRNFMTKKPYSLEKFKLNFENSTLLDGWDVNKESDNTAILFRKEG |
| | | LYYLGIMNKKYNKVFRNISSSQDEGYQKIDYKLLPGANKMLPKVFFSDK |
| | | NKEYFKPNAKLLERYKAGEHKKGDNFDLDFCHELIDFFKTSIEKHQDWK |
| | | HFAYQFSPTESYEDLSGFYREVEQQGYKISYKNIAASFIDTLVAEGKLY |
| | | FFQIYNKDFSPYSKGTPNMHTLYWRALFDEKNLADVIYKLNGQAEIFFR |
| | | KKSIEYSQEKLQKGHHHEMLKDKFAYPIIKDRRFAFDKFQFHVPITLNF |
| | | KAEGNENITPKTFEYIRSNPDNIKVIGIDRGERHLLYLSLIDAEGKIVE |
| | | QFTLNQIINSYNGKDHVIDYHAKLDAKEKDRDKARKEWGTVENIKELKE |
| | | GYLSHVIHKIATLIIEHGAVVAMEDLNFGFKRGRFKVEKQVYQKFEKAL |
| | | IDKLNYLVDKKKEPHKLGGLLNALQLTSKFQSFEKMGKQNGELFYVPAW |
| | | NTSKIDPVTGFVNLFDTRYASVEKSKAFFTKFQSICYNEAKDYFELVFD |
| | | YNDFTEKAKETRSEWTLCTYGERIVSFRNAEKNHQWDSKTIHLTTEFKN |
| | | LFGELHGNDVKEYILEQNSVEFFKSLIYLLKITLQMRNSITGTDIDYLV |
| | | SPVADEAGNFYDSRKADTSLPKDADANGAYNIARKGIMIMHRIQNAEDL |
| | | KKVNLAISNRDWLRNAQGLDK |
| ART3 | 3 | MIDLKQFIGIYPVSKTLRFELRPVGKTQEWIEKNRVLEGDEQKAADYPV |
| | | VKKLIDDYHKVCIHDSLNHVHEDWEPLKDAIEIFQKTKSDEAKKRLEAE |
| | | QAMMRKKIAAAIKDFKHFKELTAATPSDLITSVLPEFSDDGSLKSFRGF |
| | | ATYFSGFQENRNNIYSQEAISTGVPYRLVHDNFPKFLSDLEVFERIKST |
| | | CPEVINQASAELQPFLEGVMIDDIFSLDFYNSLLTQNGIDFFNQVIGGV |
| | | SEKDKQKYRGINEFSNLYRQQHKEIAASKKAMTMIPLFKQILSDRDTLS |
| | | YIPAQIRTEDELVSSITQFYDHITHFEHDGKTINVLSEIVALLGKLDTY |
| | | DPNGICITARKLTDISQKVYGKWSVIEEKMKEKAIQQYGDISVAKNKKK |
| | | VDAFLSRKAYSLSDLCFDEEISFSRYYSELPQTLNAISGYWLQFNEWCK |
| | | SDEKQKFLNNQTGTEVVKSLLDAMMELFHKCSVLVMPEEYEVDKSFYNE |
| | | FLPLYEELDTLFLLYNKVRNYLTQKPSDVKKFKLNFESPSLASGWDQNK |
| | | EMKNNAILLFKDGKSYLGVLNAKNKAKIKDAKGDVSSSSYKKMIYKLLS |
| | | DPSKDLPHKIFAKGNLDFYKPSEYILEGRELGKYKKGPNFDKKFLHDFI |
| | | DFYKAAISIDPDWSKFNFQYSPTESYDDIGMFFSEIKKQAYKIRFTDIS |
| | | EAQVNEWVDNGQLYLFQLYNKDYAEGAHGRKNLHTLYWENLFTDENLSN |
| | | LVLKLNGQAELFCRPQSIKKPVSHKIGSKMLNRRDKSGMPIPESIYRSL |
| | | YQYYNGKKKESELTVAEKQYIDQVIVKDVTHEIIKDRRYTRQEYFFHVP |
| | | LTFNANADGNEYINEHVLNYLKDNPDVNIIGIDRGERHLIYLTLINQRG |
| | | EILKQKTFNVVNSYNYQAKLEQREKERDEARKSWDSVGKIKDLKEGELS |
| | | AVIHEITNMMIENNAIVVLEDLNFGFKRGRFKVERQVYQKFEKMLIDKL |
| | | NYLSFKDREAGEEGGILRGYQMAQKFISFQRLGKQSGFLFYIPAAYTSK |
| | | IDPVSGFVNHFNFSDITNAEKRKDFLMKMDRIEMKNGNIEFTFDYRKEK |
| | | TFQTDYQNVWTVSTFGKRIVMRIDEKGYKKMVDYEPTNDIIKAFKNKGI |
| | | LLSEGSDLKALIAEIEANATNAGFYSTLLYAFQKTLQMRNSNAVTEEDY |
| | | ILSPVAKDGHQFCSTDEANKGKDAQGNWVSKLPVDADANGAYHIALKGL |
| | | YLLRNPETKKIENEKWLQEMVEKPYLE |
| ART4 | 4 | MSYNREKMEEKELGKNQNFQEFIGVSPLQKTLRNELIPTETTKKNIAQL |
| | | DLLTEDEVRAQNREKLKEMMDDYYRDVIDSTLRGELLIDWSYLFSCMRN |
| | | HLSENSKESKRELERTQDSVRSQIHDKFAERADEKDMFGASIITKLLPT |
| | | YIKQNSKYSERYDESVKIMKLYGKFTTSLTDYFETRKNIFSKEKISSAV |
| | | GYRIVEENAEIFLQNQNAYDRICKIAGLDLHGLDNEITAYVDGKTLKEV |
| | | CSDEGFAKVITQGGIDRYNEAIGAVNQYMNLLCQKNKALKPGQFKMKRL |
| | | HKQILCKGTTSFDIPKKFENDKQVYDAVNSFTEIVTKNNDLKRLLNITQ |
| | | NANDYDMNKIYVVADAYSMISQFISKKWNLIEECLLDYYSDNLPGKGNA |
| | | KENKVKKAVKEETYRSVSQLNEVIEKYYVEKTGQSVWKVESYISSLAEM |
| | | IKLELCHEIDNDEKHNLIEDDEKISEIKELLDMYMDVFHIIKVFRVNEV |
| | | LNFDETFYSEMDEIYQDMQEIVPLYNHVRNYVTQKPYKQEKYRLYFHTP |
| | | TLANGWSKSKEYDNNAIILVREDKYYLGILNAKKKPSKEIMAGKEDCSE |
| | | HAYAKMNYYLLPGANKMLPKVELSKKGIQDYHPSSYIVEGYNEKKHIKG |
| | | SKNEDIRFCRDLIDYFKECIKKHPDWNKENFEFSATETYEDISVFYREV |
| | | EKQGYRVEWTYINSEDIQKLEEDGQLFLFQIYNKDFAVGSTGKPNLHTL |
| | | YLKNLFSEENLRDIVLKLNGEAEIFFRKSSVQKPVIHKCGSILVNRTYE |
| | | ITESGTTRVQSIPESEYMELYRYENSEKQIELSDEAKKYLDKVQCNKAK |
| | | TDIVKDYRYTMDKFFIHLPITINFKVDKGNNVNAIAQQYIAEQEDLHVI |
| | | GIDRGERNLIYVSVIDMYGRILEQKSENLVEQVSSQGTKRYYDYKEKLQ |
| | | NREEERDKARKSWKTIGKIKELKEGYLSSVIHEIAQMVVKYNAIIAMED |
| | | LNYGFKRGRFKVERQVYQKFETMLISKLNYLADKSQAVDEPGGILRGYQ |
| | | MTYVPDNIKNVGRQCGIIFYVPAAYTSKIDPTTGFINAFKRDVVSTNDA |
| | | KENFLMKFDSIQYDIEKGLFKFSFDYKNFATHKLTLAKTKWDVYINGTR |
| | | IQNMKVEGHWLSMEVELTTKMKELLDDSHIPYEEGQNILDDLREMKDIT |
| | | TIVNGILEIFWLTVQLRNSRIDNPDYDRIISPVLNNDGEFFDSDEYNSY |
| | | IDAQKAPLPIDADANGAFCIALKGMYTANQIKENWVEGEKLPADCLKIE |
| | | HASWLAFMQGERG |
| ART5 | 5 | MSAVFKIKESTMKDFTHQYSLSKTLRFELKPVGETAERIEDEKNQGLKS |
| | | IVEEDRQRAEDYKKMKRILDDYHKEFIEEVLNDDIFTANEMESAFEVYR |
| | | KYMASKNDDKLKKEITEIFTDLRKKIAKAFENKSKEYCLYKGDFSKLIN |
| | | EKKTGKDKGPGKLWYWLKAKADAGVNEFGDGQTFEQAEEALAKENNEST |
| | | YFTGENQNRDNIYTDAEQQTAISYRVINENMTRYFDNCIRYSSIENKYP |
| | | ELVKQLEPLSGKFAPGNYKDYLSQTAIDIYNEAVGHKSDDINAKGINQF |
| | | INEYRQRNSIKGRELPIMSVLYKQILSDINKDLIIDKFENAGELLDAVK |
| | | TLHRELTDKKILLKIKQTLNEFLTEDNSEDIYIKSGTDLTAVSNAIWGE |
| | | WSVIPKALEMYAENITDMNAKAREKWLKREAYHLKTVQEAIEAYLKDNE |
| | | EFETRNISEYFTNFKSGENDLIQVVQSAYAKMESIFGIEDFHKDRRPVT |
| | | ESGEPGEGFRQVELVREYLDSLINVEHFIKPLHMERSGKPIELEDCNSN |
| | | FYDPLNEAYKELDVVFGIYNKVRNYVTQKPYSKDKFKINFQNSTLLDGW |
| | | DVNKESANSSVLLLKNGKYYLGVMKQGASNILNYRPEPSDSKNKINAKK |
| | | QLSEIALAGATDDYYEKMIYKLLPDPAKMLPKVFFSAKNIEFYNPSQEI |
| | | IYIRENGLFKKDAGDKESLKKWIGFMKTSLLKHPEWGSYFNFEFEPAED |
| | | YQDISIFYKQVAEQGYSVTFDKIKTSYIEEKVASGELYLFEIYNKDFSP |
| | | HSKGRPNLHTMYWKSLFEKENLQNLVTKLNGEAEVFFRQHSIKRNEKVV |
| | | HRANRPIQNKNPLTEKKQSIFEYDLVKDRRFTKDKFFLHCPITLNFKEA |
| | | GPGRFNDKVNKYIAGNPDIRIIGIDRGERHLLYYSLIDQSGRIVEQGTL |
| | | NQITSTLNSGGREIPKTTDYRGLLDTKEKERDKARKSWSMIENIKELKS |
| | | GYLSHIVHKLAKLMVKNNAVVVLEDLNFGFKRGRFKVEKQVYQKFEKAL |
| | | IEKLNYLVFKDARPAEPGHYLNAYQLTAPLESFKKLGKQSGFIYYVPAW |
| | | NTSKIDPVTGFVNQFYIEKNSMQYLKNFFGKFDSIRENPDKNYFEFGED |
| | | YKNFHNKAAKSKWTICTHGDKRSWYNRKQRKLEIHNVTENLASLLSGKG |
| | | INFADGGSIKDKILSVDDASFFKSLAFNFKLTAQLRHTFEDNGEEIDCI |
| | | ISPVAAADGTFFCSETAKKLNMELPHDADANGAYNIARKGLMVLRQIRE |
| | | SGKPKPISNADWLDFAQQNED |
| ART6 | 6 | MQERKKISHLTHRNSVQKTIRMQLNPVGKTMDYFQAKQILENDEKLKEN |
| | | YQKIKEIADRFYRNLNEDVLSKTGLDKLKDYAEIYYHCNTDAERKRLDE |
| | | CASELRKEIVKNFKNRDEYNKLFNKKMIEIVLPQHLKNEDEKEVVASFK |
| | | NFTTYFTGFFTNRKNMYSDGEESTAIAYRCINENLPKHLDNVKAFEKAI |
| | | SKLSKNAIDDLDATYSGLCGTNLYDVFTVDYFNFLLPQSGITEYNKIIG |
| | | GYTTSDGTKVKGINEYINLYNQQVSKRDKIPNLKILYKQILSESEKVSE |
| | | IPPKFEDDNELLSAVSEFYANDETFDGMPLKKAIDETKLLFGNLDNSSL |
| | | NGIYIQNDRSVINLSNSMFGSWSVIEDLWNKNYDSVNSNSRIKDIQKRE |
| | | DKRKKAYKAEKKLSLSFLQVLISNSENDEIREKSIVNYYKTSLMQLTDN |
| | | LSDKYNEAAPLLNKSYANEKGLKNDDKSISLIKNFLDAIKEIEKFIKPL |
| | | SETNITGEKNDLFYSQFTPLLDNISRIDILYDKVRNYVTQKPFSTDKIK |
| | | LNFGNSQLLNGWDRNKEKDCGAVWLCRDEKYYLAIIDKSNNSILENIDF |
| | | QDCDENDCYEKIIYKLLPGPNKMLPKVFFSEKCKKLLSPSDEILKIRKN |
| | | GTFKKGDKFSLDDCHKLIDFYKESFKKYPNWLIYNFKFKNTNEYNDIRE |
| | | FYNDVASQGYNISKMKIPTSFIDKLVDEGKIYLFQLYNKDESPHSKGTP |
| | | NLHTLYFKMLFDERNLEDVVYKLNGEAEMFYRPASIKYDKPTHPKNTPI |
| | | KNKNTLNDKKTSTFPYDLIKDKRYTKWQFSLHFPITMNFKAPDRAMIND |
| | | DVRNLLKSCNNNFIIGIDRGERNLLYVSVIDSNGAIIYQHSLNIIGNKE |
| | | KGKTYETNYREKLATREKERTEQRRNWKAIESIKELKEGYISQAVHVIC |
| | | QLVVKYDAIIVMEKLTDGFKRGRTKFEKQVYQKFEKMLIDKLNYYVDKK |
| | | LDPDEGGGLLHAYQLTNKLESFDKLGMQSGFIFYVRPDFTSKIDPVTGF |
| | | VNLLYPRYENIDKAKDMISREDDIGYNAGEDFFEFDIDYDKFPKTASDY |
| | | RKRWTICTNGERIEAFRNPAKNNEWSYRTIILAEKFKELFDNNSINYRD |
| | | SDDLKAEILSQTKGKFFEDFFKLLRLTLQMRNSNPETGEDRILSPVKDK |
| | | NGNFYDSSKYDEKSKLPCDADANGAYNIARKGLWIVEQFKKSDNVSTVG |
| | | PVIHNDKWLKFVQENDMANN |
| ART7 | 7 | MNILKENYMKEIKELTGLYSLTKTIGVELKPVGKTQELIEAKKLIEQDD |
| | | QRAEDYKIVKDIIDRYHKDFIDKCLNCVKIKKDDLEKYVSLAENSNRDA |
| | | EDFDKIKTKMRNQITEAFRKNSLFTNLFKKNLIKEYLPAFVSEEEKSVV |
| | | NKFSKFTTYFDAFNDNRKNLYSGDAKSGTIAYRLIHENLPMELDNIASF |
| | | NAISGIGVNEYFSSIETEFTDTLEGKRLTEFFQIDFENNTLTQKKIGNY |
| | | NYIVGAVNKAVNLYKQQHKTVRVPLLKPLYKMILSDRVTPSWLPERFES |
| | | DEEMLTAIKAAYESLREVLVGDNDESLRNLLLNIEHYDLEHIYIANDSG |
| | | LTSISQKIFGCYDTYTLAIKDQLQRDYPATKKQREAPDLYDERIDKLYK |
| | | KVGSFSIAYLNRLVDAKGHFTINEYYKQLGAYCREEGKEKDDFFKRIDG |
| | | AYCAISHLFFGEHGEIAQSDSDVELIQKLLEAYKGLQRFIKPLLGHGDE |
| | | ADKDNEFDAKLRKVWDELDIITPLYDKVRNWLSRKIYNPEKIKLCFENN |
| | | GKLLSGWVDSRTKSDNGTQYGGYIFRKKNEIGEYDFYLGISADTKLFRR |
| | | DAAISYDDGMYERLDYYQLKSKTLLGNSYVGDYGLDSMNLLSAFKNAAV |
| | | KFQFEKEVVPKDKENVPKYLKRLKLDYAGFYQILMNDDKVVDAYKIMKQ |
| | | HILATLTSSIRVPAAIELATQKELGIDELIDEIMNLPSKSFGYFPIVTA |
| | | AIEEANKRENKPLFLFKMSNKDLSYAATASKGLRKGRGTENLHSMYLKA |
| | | LLGMTQSVFDIGSGMVFFRHQTKGLAETTARHKANEFVANKNKLNDKKK |
| | | SIFGYEIVKNKRFTVDKYLFKLSMNLNYSQPNNNKIDVNSKVREIISNG |
| | | GIKNIIGIDRGERNLLYLSLIDLKGNIVMQKSLNILKDDHNAKETDYKG |
| | | LLTEREGENKEARRNWKKIANIKDLKRGYLSQVVHIISKMMVEYNAIVV |
| | | LEDLNPGFIRGRQKIERNVYEQFERMLIDKLNFYVDKHKGANETGGLLH |
| | | ALQLTSEFKNFKKSEHQNGCLFYIPAWNTSKIDPATGFVNLENTKYTNA |
| | | VEAQEFFSKFDEIRYNEEKDWFEFEFDYDKFTQKAHGTRTKWTLCTYGM |
| | | RLRSFKNSAKQYNWDSEVVALTEEFKRILGEAGIDIHENLKDAICNLEG |
| | | KSQKYLEPLMQFMKLLLQLRNSKAGTDEDYILSPVADENGIFYDSRSCG |
| | | DQLPENADANGAYNIARKGLMLIEQIKNAEDLNNVKFDISNKAWINFAQ |
| | | QKPYKNG |
| ART8 | 8 | MAKENIFNELTGKYQLSKTLRLELKPVGNTQQMLKDEDVFEKDRIIREK |
| | | YRETRPHFDRLHREFIEQALKNQKLSDLGKYFQCLAKLQNNKKDKEAQE |
| | | EFKRISQNLRKEVNDLFKIDPLFGEGVFALLKEKYGEKDDAFLREQDGQ |
| | | YVLDENKKKISIFDSWKGFTGYFTKFQETRKNFYKDDGTATAVATRIID |
| | | QNLKRFCENIQIFKSIQKKVDFKEVEDNFSVDLEDIFSLGFYSSCFLQE |
| | | GIDVYNKILGGEPKTTGEKLRGLNELINRYRQDHKGEKLPFFKMLDKQI |
| | | LSEKEKFIESIEDDEELLKTLKEFYSSAEEKTTVLKELENDFIKNNENY |
| | | DLSEIYISREALNTISHRWVSAATLPEFEKSVYEVMKKDKPSGLSFDKD |
| | | DNSYKFPDFIALSYIKGSFEKLSGEKLWKDGYFRDETRNGDKGFLIGNE |
| | | SLWTQFIKIFEFEFNSLFEAKNTERSVGYYHFKKDFEKIITNDFSVNPE |
| | | DKVIIREFADNVLAIYQMAKYFAIEKKRKWMDQYDTGDFYNHPDFGYKT |
| | | KFYDNAYEKIVKARMLLQSYLTKKPFSTDKWKLNFECGYLLNGWSSSEN |
| | | TYGSLLFRTGNEYYLGVVNGSALRTEKIKRLTGNITEANSCHKMVYDFQ |
| | | KPDNKNVPRIFIRSKGDKFAPAVSELNLPVDSILEIYDKGLFKTENKNS |
| | | PFFKPSLKKLIDYFKLGFSRHASYKHYQFKWKDSSEYKNISEFYNDTIR |
| | | SCYQIKWEELNFEEVKKLTNSKDLFLFQIYNKDFSEKSTGNKNLHSIYF |
| | | DGLFLDNNINAQDGVILKLSGGGEIFFRPKTDVKKLGSRTDTKGKLVIK |
| | | NKRYSQDKIFLHFPIELNYSNTQESNENKLVRNFLADNPDINIIGVDRG |
| | | EKHLIYYAGIDQKGNTLKDKDDKDVLGSLNEINGVNYYKLLEERAKARE |
| | | KARQDWQNIQGIKDLKMGYISLVVRKLADLIIEYNAILVLEDLNMRFKQ |
| | | IHGGIEKSVYQQLEKALIEKLNFLVNKGEKDPERAGHLLRAYQLTAPES |
| | | TFKDMGKQTGVLFYTQASYTSKTCPQCGFRPNIKLHFDNLENAKKMLEK |
| | | INIVYKDNHFEIGYKVSDFTKTEKTSRGNILYGDRQGKDTFVISSKAAI |
| | | RYKWFARNIKNNELNRGESLKEHTEKGVTIQYDITECLKILYEKNGIDH |
| | | SGDITKQSIRSELPAKFYKDLLFYLYLLTNTRSSISGTEIDYINCPDCG |
| | | FHSEKGFNGCIFNGDANGAYNIARKGMLILKKINQYKDQHHTMDKMGWG |
| | | DLFIGIEEWDKYTQVVSRS |
| ART9 | 9 | MKEIKELTGLYSLTKTIGVELKPVGKTQELIEAKKLIEQDDQRAEDYKI |
| | | VKDIIDRYHKDFIDKCLNCVKIKKDDLEKYVSLAENSNRDAEDFDKIKT |
| | | KMRNQITEAFRKNSLFTNLFKKNLIKEYLPAFVSEEEKSVVNKFSKFTT |
| | | YFDAFNDNRKNLYSGDAKSGTIAYRLIHENLPMFLDNIASFNAISGIGV |
| | | NEYFSSIETEFTDTLEGKRLTEFFQIDFENNTLTQKKIGNYNYIVGAVN |
| | | KAVNLYKQQHKTVRVPLLKPLYKMILSDRVTPSWLPERFESDEEMLTAI |
| | | KAAYESLREVLVGDNDESLRNLLLNIEHYDLEHIYIANDSGLTSISQKI |
| | | FGCYDTYTLAIKDQLQRDYPATKKQREAPDLYDERIDKLYKKVGSFSIA |
| | | YLNRLVDAKGHFTINEYYKQLGAYCREEGKEKDDFFKRIDGAYCAISHL |
| | | FFGEHGEIAQSDSDVELIQKLLEAYKGLQRFIKPLLGHGDEADKDNEFD |
| | | AKLRKVWDELDIITPLYDKVRNWLSRKIYNPEKIKLCFENNGKLLSGWV |
| | | DSRTKSDNGTQYGGYIFRKKNEIGEYDFYLGISADTKLFRRDAAISYDD |
| | | GMYERLDYYQLKSKTLLGNSYVGDYGLDSMNLLSAFKNAAVKFQFEKEV |
| | | VPKDKENVPKYLKRLKLDYAGFYQILMNDDKVVDAYKIMKQHILATLTS |
| | | SIRVPAAIELATQKELGIDELIDEIMNLPSKSFGYFPIVTAAIEEANKR |
| | | ENKPLFLFKMSNKDLSYAATASKGLRKGRGTENLHSMYLKALLGMTQSV |
| | | FDIGSGMVFFRHQTKGLAETTARHKANEFVANKNKLNDKKKSIFGYEIV |
| | | KNKRFTVDKYLFKLSMNLNYSQPNNNKIDVNSKVREIISNGGIKNIIGI |
| | | DRGERNLLYLSLIDLKGNIVMQKSLNILKDDHNAKETDYKGLLTEREGE |
| | | NKEARRNWKKIANIKDLKRGYLSQVVHIISKMMVEYNAIVVLEDLNPGF |
| | | IRGRQKIERNVYEQFERMLIDKLNFYVDKHKGANETGGLLHALQLTSEF |
| | | KNFKKSEHQNGCLFYIPAWNTSKIDPATGFVNLENTKYTNAVEAQEFFS |
| | | KFDEIRYNEEKDWFEFEFDYDKFTQKAHGTRTKWTLCTYGMRLRSFKNS |
| | | AKQYNWDSEVVALTEEFKRILGEAGIDIHENLKDAICNLEGKSQKYLEP |
| | | LMQFMKLLLQLRNSKAGTDEDYILSPVADENGIFYDSRSCGDQLPENAD |
| | | ANGAYNIARKGLMLIEQIKNAEDLNNVKEDISNKAWLNFAQQKPYKNG |
| ART10 | 10 | MNFQPFFQKFVHLYPISKTLRFELIPQGATQKFISEKQVLLQDEIRARK |
| | | YPEMKQAIDGYHKDFIQRALSNIDSQVFEQALNTFEDLFLRSQAERATD |
| | | AYKKDFETAQTKLRELIVHSFEKGEFKQEYKSLFDKNLITNLLKPWVEQ |
| | | QNQIGDSNYTYHEDENKFTTYFLGFHENRKNIYSKDPHKTALAYRLIHE |
| | | NLPKFLENNKILLKIQNDHPSLWEQLQTLNQTMPQLFDGWDFSQLMQVS |
| | | FFSNTLTQTGIDQYNTIIGGISEGENRQKIQGINELINLYNQKQDKKNR |
| | | VAKLKQLYKQILSDRSTLSFLPEKFVDDTELYHAINMFYLEHLHHQSMI |
| | | NGHSYTLLERVQLLINELANYDLSKVYLAPNQLSTVSHQMFGDEGYIGR |
| | | ALNYYYMQVIQPDYEQLLASAKTTKKIEATEKLKTIFLDTPQSLVVIQA |
| | | AIDEYIQLQPSTKPHTQLTDFIISLLKQYETVADDQSIKVINVESDIEG |
| | | KYSCIKGLVNTKSESKREVLQDEKLATDIKAFMDAVNNVIKLLKPFSLN |
| | | EKLVASVEKDARFYSDFEEIYQSLLIFVPLYNKVRNYITQKPYSTEKFK |
| | | LNFNKPTLLSGWDANKEADNLSILLRKNGNYYLAIMDTAKGANKAFEPK |
| | | TLNQLKVDDTTDCYEKMVYKLLSGPSKMFPKAFKAKNNEGNYYPTPELL |
| | | TSYNNNEHLKNDKNFTLASLHAYIDWCKEYINRNPSWHQFNFKESPTQS |
| | | FQDISQFYSEVSSQSYKVHFQTIPSDYIDQLVAEGKLYLFQIYNKDFSP |
| | | NAKGKENLHTLYFKALFSDENLKQPVFKLSGEAEMFYRPASLQLANTTI |
| | | HKAGEPMAAKNPLTPNATRTLAYDIIKDRRFTTDKYLLHVPISLNFHAQ |
| | | ESMSIKKHNDLVRQMIKHNHQDLHVIGIDRGEKHLLYVSVIDLKGNIVY |
| | | QESLNSIKSEAQNFETPYHQLLQHREEGRAQARTAWGKIENIKELKDGY |
| | | LSQVVHRIQQLILKYNAIVMLEDLNFGFKRGRFKIEKQIYQKFEKALIH |
| | | KLNYVVDKSTQADELGGVRKAYQLTAPFESFEKLGKQSGVLFYVPAWNT |
| | | SKIDPVTGFVDLLKPKYENLDKAQAFFNAFDSIHYNAQKNYFEFKVNLK |
| | | QFAGLKAQAAQAEWTICSYGDERHVYQKKNAQQGETVIVNVTEELKVLF |
| | | AKNNIEVAQSVELKETICTQTQVDFFKRLMWLLQVLLALRYSSSKDKLD |
| | | YILSPVANAQGEFFDSRHASVQLPQDSDANGAYHIALKGLWVIEQLKAA |
| | | DNLDKVKLAISNDDWLHFAQQKPYLA |
| ART11 | 11 | MYYQGLTKLYPISKTIRNELIPVGKTLEHIRMNNILEADIQRKSDYERV |
| | | KKLMDDYHKQLINESLQDVHLSYVEEAADLYLNASKDKDIVDKESKCQD |
| | | KLRKEIVNLLKSHENFPKIGNKEIIKLLQSLSDTEKDYNALDSFSKFYT |
| | | YFTSYNEVRKNLYSDEEKSSTAAYRLINENLPKELDNIKAYSIAKSAGV |
| | | RAKELTEEEQDCLFMTETFERTLTQDGIDNYNELIGKLNFAINLYNQQN |
| | | NKLKGFRKVPKMKELYKQILSEREASFVDEFVDDEALLTNVESFSAHIK |
| | | EFLESDSLSRFAEVLEESGGEMVYIKNDTSKTTFSNIVEGSWNVIDERL |
| | | AEEYDSANSKKKKDEKYYDKRHKELKKNKSYSVEKIVSLSTETEDVIGK |
| | | YIEKLQADIIAIKETREVFEKVVLKEHDKNKSLRKNTKAIEAIKSFLDT |
| | | IKDFERDIKLISGSEHEMEKNLAVYAEQENILSSIRNVDSLYNMSRNYL |
| | | TQKPFSTEKFKLNFNRATLLNGWDKNKETDNLGILLVKEGKYYLGIMNT |
| | | KANKSFVNPPKPKTDNVYHKVNYKLLPGPNKMLPKVFFAKSNLEYYKPS |
| | | EDLLAKYQAGTHKKGENFSLEDCHSLISFFKDSLEKHPDWSEFGFKFSD |
| | | TKKYDDLSGFYREVEKQGYKITYTDIDVEYIDSLVEKDELYLFQIYNKD |
| | | FSPYSKGNYNLHTLYLTMLFDERNLRNVVYKLNGEAEVFYRPASIGKDE |
| | | LIIHKSGEEIKNKNPKRAIDKPTSTFEYDIVKDRRYTKDKFMLHIPVTM |
| | | NFGVDETRRFNEVVNDAIRGDDKVRVIGIDRGERNLLYVVVVDSDGTIL |
| | | EQISLNSIINNEYSIETDYHKLLDEKEGDRDRARKNWTTIENIKELKEG |
| | | YLSQVVNVIAKLVLKYDAIICLEDLNFGFKRGRQKVEKQVYQKFEKMLI |
| | | DKLNYLVIDKSRSQENPEEVGHVLNALQLTSKFTSFKELGKQTGIIYYV |
| | | PAYLTSKIDPTTGFANLFYVKYESVEKSKDFFNRFDSICENKVAGYFEF |
| | | SFDYKNFTDRACGMRSKWKVCINGERIIKYRNEEKNSSFDDKVIVLTEE |
| | | FKKLFNEYGIAFNDCMDLTDAINAIDDASFFRKLTKLFQQTLQMRNSSA |
| | | DGSRDYIISPVENDNGEFFNSEKCDKSKPKDADANGAFNIARKGLWVLE |
| | | QLYNSSSGEKLNLAMTNAEWLEYAQQHTI |
| ART12 | 12 | MAKNFEDFKRLYPLSKTLRFEAKPIGATLDNIVKSGLLEEDEHRAASYV |
| | | KVKKLIDEYHKVFIDRVLDNGCLPLDDKGDNNSLAEYYESYVSKAQDED |
| | | AIKKEKEIQQNLLSIIAKKLTDDKAYANLFGNKLIESYKDKADKTKLID |
| | | SDLIQFINTAESTQLVSMSQDEAKELVKEFWGFTTYFEGFFKNRKNMYT |
| | | PEEKSTGIAYRLINENLPKFIDNMEAFKKAIARPEIQANMEELYSNFSE |
| | | YLNVESIQEMFLLDYYNMLLTQKQIDVYNAIIGGKTDDEHDVKIKGINE |
| | | YINLYNQQHKDDKLPKLKALFKQILSDRNAISWLPEEFNSDQEVLNAIK |
| | | DCYERLAENVLGDKVLKSLLGSLADYSLDGIFIRNDLQLTDISQKMEGN |
| | | WGVIQNAIMQNIKHVAPARKHKESEEDYEKRIAGIFKKADSFSISYIND |
| | | CLNEADPNNAYFVENYFATFGAVNTPTMQRENLFALVQNAYTEVAALLH |
| | | SDYPTVKHLAQDKANVSKIKALLDAIKSLQHFVKPLLGKGDESDKDERF |
| | | YGELASLWAELDTVTPLYNMIRNYMTRKPYSQKKIKLNFENPQLLGGWD |
| | | ANKEKDYATIILRRNGLYYLAIMDKDSRKLLGKAMPSDGECYEKMVYKF |
| | | FKDVTTMIPKCSTQLKDVQAYFKVNTDDYVLNSKAFNRPLTITKEVEDL |
| | | NNVLYGKYKKFQKGYLTATGDNVGYTHAVNVWIKFCMDFLDSYDSTCIY |
| | | DESSLKPESYLSLDSFYQDVNLLLYKLSFTDVSASFIDQLVEEGKMYLE |
| | | QIYNKDFSEYSKGTPNMHTLYWKALFDERNLADVVYKLNGQAEMFYRKK |
| | | SIENTHPTHPANHPILNKNKDNKKKESLFEYDLIKDRRYTVDKEMFHVP |
| | | ITMNFKSSGSENINQDVKAYLRHADDMHIIGIDRGERHLLYLVVIDLQG |
| | | NIKEQFSLNEIVNDYNGNTYHTNYHDLLDVREDERLKARQSWQTIENIK |
| | | ELKEGYLSQVIHKITQLMVRYHAIVVLEDLSKGFMRSRQKVEKQVYQKF |
| | | EKMLIDKLNYLVDKKTDVSTPGGLLNAYQLTCKSDSSQKLGKQSGFLFY |
| | | IPAWNTSKIDPVTGFVNLLDTHSLNSKEKIKAFFSKFDAIRYNKDKKWF |
| | | EFNLDYDKFGKKAEDTRTKWTLCTRGMRIDTERNKEKNSQWDNQEVDLT |
| | | TEMKSLLEHYYIDIHGNLKDAISTQTDKAFFTGLLHILKLTLQMRNSIT |
| | | GTETDYLVSPVADENGIFYDSRSCGDQLPENADANGAYNIARKGLMLVE |
| | | QIKDAEDLDNVKFDISNKAWLNFAQQKPYKNG |
| ART13 | 13 | MAKNFEDFKRLYSLSKTLRFEAKPIGATLDNIVKSGLLDEDEHRAASYV |
| | | KVKKLIDEYHKVFIDRVLDDGCLPLENKGNNNSLAEYYESYVSRAQDED |
| | | AKKKFKEIQQNLRSVIAKKLTEDKAYANLFGNKLIESYKDKEDKKKIID |
| | | SDLIQFINTAESTQLDSMSQDEAKELVKEFWGFVTYFYGFFDNRKNMYT |
| | | AEEKSTGIAYRLVNENLPKFIDNIEAFNRAITRPEIQENMGVLYSDESE |
| | | YLNVESIQEMFQLDYYNMLLTQKQIDVYNAIIGGKTDDEHDVKIKGINE |
| | | YINLYNQQHKDDKLPKLKALFKQILSDRNAISWLPEEFNSDQEVLNAIK |
| | | DCYERLAENVLGDKVLKSLLGSLADYSLDGIFIRNDLQLTDISQKMEGN |
| | | WGVIQNAIMQNIKRVAPARKHKESEEDYEKRIAGIFKKADSFSISYIND |
| | | CLNEADPNNAYFVENYFATFGAVNTPTMQRENLFALVQNAYTEVAALLH |
| | | SDYPTVKHLAQDKANVSKIKALLDAIKSLQHFVKPLLGKGDESDKDERF |
| | | YGELASLWAELDTVTPLYNMIRNYMTRKPYSQKKIKLNFENPQLLGGWD |
| | | ANKEKDYATIILRRNGLYYLAIMDKDSRKLLGKAMPSDGECYEKMVYKF |
| | | FKDVTTMIPKCSTQLKDVQAYFKVNTDDYVLNSKAFNKPLTITKEVEDL |
| | | NNVLYGKYKKFQKGYLTATGDNVGYTHAVNVWIKFCMDELNSYDSTCIY |
| | | DFSSLKPESYLSLDAFYQDANLLLYKLSFARASVSYINQLVEEGKMYLF |
| | | QIYNKDFSEYSKGTPNMHTLYWKALFDERNLADVVYKLNGQAEMFYRKK |
| | | SIENTHPTHPANHPILNKNKDNKKKESLFDYDLIKDRRYTVDKEMFHVP |
| | | ITMNFKSVGSENINQDVKAYLRHADDMHIIGIDRGERHLLYLVVIDLQG |
| | | NIKEQYSLNEIVNEYNGNTYHTNYHDLLDVREEERLKARQSWQTIENIK |
| | | ELKEGYLSQVIHKITQLMVRYHAIVVLEDLSKGEMRSRQKVEKQVYQKF |
| | | EKMLIDKLNYLVDKKTDVSTPGGLLNAYQLTCKSDSSQKLGKQSGFLFY |
| | | IPAWNTSKIDPVTGFVNLLDTHSLNSKEKIKAFFSKFDAIRYNKDKKWF |
| | | EFNLDYDKFGKKAEDTRTKWTLCTRGMRIDTERNKEKNSQWDNQEVDLT |
| | | TEMKSLLEHYYIDIHGNLKDAISAQTDKAFFTGLLHILKLTLQMRNSIT |
| | | GTETDYLVSPVADENGIFYDSRSCGNQLPENADANGAYNIARKGLMLIE |
| | | QIKNAEDLNNVKFDISNKAWLNFAQQKPYKNG |
| ART14 | 14 | MAKNFEDFKRLYSLSKTLRFEAKPIGATLDNIVKSDLLDEDEHRAASYV |
| | | KVKKLIDEYHKVFIDRVLDDGCLPLENKGNNNSLAEYYESYVSRAQDED |
| | | AKKKFKEIQQNLRSVIAKKLTEDKAYANLFGNKLIESYKDKEDKKKIID |
| | | SDLIQFINTAESTQLDSMSQDEAKELVKEFWGFVTYFYGFFDNRKNMYT |
| | | AEEKSTGIAYRLVNENLPKFIDNIEAFNRAITRPEIQENMGVLYSDFSE |
| | | YLNVESIQEMFQLDYYNMLLTQKQIDVYNAIIGGKTDDEHDVKIKGIND |
| | | YINLYNQKHKDDKLPKLKALFKQILSDRNAISWLPEEFNSDQEVLNAIK |
| | | DCYERLSENVLGDKVLKSMLGSLADYSLDGIFIRNDLQLTDISQKMEGN |
| | | WSVIQNAIMQNIKHVAPARKHKESEEEYENRIAGIFKKADSFSISYIDA |
| | | CLNETDPNNAYFVENYFATLGAVDTPTMQRENLFALVQNAYTEITALLH |
| | | SDYPTEKNLAQDKANVAKIKALLDAIKSLQHFVKPLLGKGDESDKDERF |
| | | YGELASLWAELDTMTPLYNMIRNYMTRKPYSQKKIKLNFENPQLLGGWD |
| | | ANKEKDYATIILRRNGLYYLAIMNKDSKKLLGKAMPSDGECYEKMVYKL |
| | | LPGANKMLPKVFFAKSRMEDFKPSKELVEKYYNGTHKKGKNFNIQDCHN |
| | | LIDYFKQSIDKHEDWSKFGFKFSDTSTYEDLSGFYREVEQQGYKLSFAR |
| | | VSVSYINQLVEEGKMYLFQIYNKDFSEYSKGTPNMHTLYWKALFDERNL |
| | | ADVVYKLNGQAEMFYRKKSIENTHPTHPANHPILNKNKDNKKKESLFGY |
| | | DLIKDRRYTVDKFLFHVPITMNFKSSGSENINQDVKAYLRHADDMHIIG |
| | | IDRGERHLLYLVVIDLQGNIKEQFSLNEIVNDYNGNTYHTNYHDLLDVR |
| | | EDERLKARQSWQTIENIKELKEGYLSQVIHKITQLMVKYHAIVVLEDLN |
| | | MGFMRGRQKVEKQVYQKFEKMLIEKLNYLVDKKADASVSGGLLNAYQLT |
| | | SKEDSFQKLGKQSGFLFYIPAWNTSKIDPVTGFVNLLDTRYQNVEKAKS |
| | | FFSKFDAIRYNKDKEWFEFNLDYDKFGKKAEGTRTKWTLCTRGMRIDTF |
| | | RNKEKNSQWDNQEVDLTAEMKSLLEHYYIDIHSNLKDAISAQTDKAFFT |
| | | GLLHILKLTLQMRNSITGTETDYLVSPVVDENGIFYDSRSCGDELPENA |
| | | DANGAYNIARKGLMMIEQIKDAKDLDNLKFDISNKAWLNFAQQKPYKNG |
| ART15 | 15 | MLFQDFTHLYPLSKTVRFELKPIGRTLEHIHAKNFLSQDETMADMYQKV |
| | | KVILDDYHRDFIADMMGEVKLTKLAEFYDVYLKFRKNPKDDELQKQLKD |
| | | LQAVLRKESVKPIGNGGKYKAGHDRLFGAKLFKDGKELGDLAKEVIAQE |
| | | GKSSPKLAHLAHFEKFSTYFTGFHDNRKNMYSDEDKHTAIAYRLIHENL |
| | | PRFIDNLQILTTIKQKHSALYDQIINELTASGLDVSLASHLDGYHKLLT |
| | | QEGITAYNRIIGEVNGYTNKHNQICHKSERIAKLRPLHKQILSDGMGVS |
| | | FLPSKFADDSEMCQAVNEFYRHYADVFAKVQSLEDGEDDHQKDGIYVEH |
| | | KNLNELSKQAFGDFALLGRVLDGYYVDVVNPEFNERFAKAKTDNAKAKL |
| | | TKEKDKFIKGVHSLASLEQAIKHHTARHDDESVQAGKLGQYFKHGLAGV |
| | | DNPIQKIHNNHSTIKGFLERERPAGERALPKIKSGKNPEMTQLRQLKEL |
| | | LDNALNVAHFAKLLMTKTTLDNQDGNFYGEFGVLYDELAKIPTLYNKVR |
| | | DYLSQKPFSTEKYKLNFGNPTLLNGWDLNKEKDNFGVILQKDGCYYLAL |
| | | LDKAHKKVFDNAPNTGKNVYQKMIYKLLPGPNKMLPRVFFAKSNLDYYN |
| | | PSAELLDKYAQGTHKKGDNFNLKDCHALIDFFKAGINKHPEWQNFGFKE |
| | | SPTSSYRDLSDFYREVEPQGYQVKFVDINADYIDELVEQGQLYLFQIYN |
| | | KDFSPKAHGKPNLHTLYFRALFSEDNLANPIYKLNGEAQIFYRKASLGM |
| | | NETTIHRAGEILENKNPDNPKERVFTYDIIKDRRYTQDKFMLHVPITMN |
| | | FGVQGMTIKEFNKKVNQSIRQYDDVNVIGIDRGERHLLYLTVINSKGEI |
| | | LEQRSLNDITTASANGTQMTTPYHKILDKREIERLNARVGWGEIETIKE |
| | | LKSGYLSHVVHQVSQLMLKYNAIVVLEDLNFGFKRGRFKVEKQIYQNFE |
| | | NALIKKLNHLELKDKADDEIGSYKNALQLTNNFTDLKNIGKQTGELFYV |
| | | PAWNTSKIDPETGFVDLLKPRYENIAQSQAFFGKFDKICYNADKDYFEF |
| | | HIDYAKFTDKAKNSRQTWTICSHGDKRYVYDKTANQNKGATKGINVNDE |
| | | LKSLFARYHINEKQPNLVMDICQNNDKEFHKSLMYLLKTLLALRYSNAS |
| | | SDEDFILSPVANDEGVFFNSALADDTQPQNADANGAYHIALKGLWLLNE |
| | | LKNSDDLNKVKLAIDNQTWLNFAQNR |
| ART16 | 16 | MLFQDFTHLYPLSKTVRFELKPIGKTLEHIHAKNFLSQDETMADMYQKV |
| | | KAILDDYHRDFITKMMSEVTLTKLPEFYEVYLALRKNPKDDTLQKQLTE |
| | | IQTALREEVVKPIDSGGKYKAGYERLFGAKLFKDGKELGDLAKFVIAQE |
| | | GESSPKLPQIAHFEKFSTYFTGFHDNRKNMYSSDDKHTAIAYRLIHENL |
| | | PRFIDNLQILVTIKQKHSVLYDQIVNELNANGLDVSLASHLDGYHKLLT |
| | | QEGITAYNRIIGEVNSYTNKHNQICHKSERIAKLRPLHKQILSDGMGVS |
| | | FLPSKFADDSEMCQAVNEFYRHYAHVFAKVQSLEDREDDYQKDGIYVEH |
| | | KNLNELSKQAFGDFALLGRVLDGYYVDVVNPEFNDKFAKAKTDNAKEKL |
| | | TKEKDKFIKGVHSLASLEQAIEHYIAGHDDESVQAGKLGQYFKHGLAGV |
| | | DNPIQKIHNSHSTIKGFLERERPAGERTLPKIKSDKSLEMTQLRQLKEL |
| | | LDNALNVVHFAKLLTTKTTLDNQDGNFYGEFGALYDELAKIATLYNKVR |
| | | DYLSQKPFSTEKYKLNFGNPTLLNGWDLNKEKDNFGVILQKDGCYYLAL |
| | | LDKAHKKVFDNAPNTGKSVYQKMVYKLLPGPNKMLPKVFFAKSNLDYYN |
| | | PSAELLDKYAQGTHKKGDNENLKDCHALIDFFKASINKHPEWQHFGFEF |
| | | SLTSSYQDLSDFYREVEPQGYQVKFVDIDADYIDELVEQGQLYLFQIYN |
| | | KDFSPKAHGKPNLHTLYFKALFSEDNLANPIYKLNGEAEIFYRKASLDM |
| | | NETTIHRAGEVLENKNPDNPKERQFVYDIIKDKRYTQDKEMLHVPITMN |
| | | FGVQGMTIKEFNKKVNQSIQQYDEVNVIGIDRGERHLLYLTVINSKGEI |
| | | LEQRSLNDIITTSANGTQMTTPYHKILDKREIERLNARVGWGEIETIKE |
| | | LKSGYLSHVVHQISQLMLKYNAIVVLEDLNFGFKRGRFKVEKQIYQNFE |
| | | NALIKKLNHLVLKDKADNEIGSYKNALQLTNNFTDLKSIGKQTGFLFYV |
| | | PAWNTSKIDPVTGFVDLLKPRYENIAQSQAFFDKEDKICYNADKGYFEF |
| | | HIDYAKFTDKAKNSRQIWTICSHGDKRYVYDKTANQNKGATIGINVNDE |
| | | LKSLFARYRINDKQPNLVMDICQNNDKEFHKSLTYLLKALLALRYSNAS |
| | | SDEDFILSPVANDKGVFFNSALADDTQPQNADANGAYHIALKGLWLLNE |
| | | LKNSDDLDKVKLAIDNQTWLNFAQNR |
| ART17 | 17 | MLFQDFTHLYPLSKTVRFELKPIGKTLEHIHAKNFLSQDETMADMYQKV |
| | | KAILDDYHRDFITKMMSEVTLTKLPEFYEVYLALRKNPKDDTLQKQLTE |
| | | IQTALREEVVKPIDSGGKYKAGYERLFGAKLFKDGKELGDLAKEVIAQE |
| | | GESSPKLPQIAHFEKFSTYFTGFHDNRKNMYSSDDKHTAIAYRLIHENL |
| | | PRFIDNLQILVTIKQKHSVLYDQIVNELNANGLDVSLASHLDGYHKLLT |
| | | QEGITAYNRIIGEVNSYTNKHNQICHKSERIAKLRPLHKQILSDGMGVS |
| | | FLPSKFADDSEMCQAVNEFYRHYAHVFAKVQSLEDREDDYQKDGIYVEH |
| | | KNLNELSKQAFGDFALLGRVLDGYYVDVVNPEENDKFAKAKTDNAKEKL |
| | | TKEKDKFIKGVHSLASLEQAIEHYIAGHDDESVQAGKLGQYFKHGLAGV |
| | | DNPIQKIHNSHSTIKGFLERERPAGERTLPKIKSDKSLEMTQLRQLKEL |
| | | LDNALNVVHFAKLLTTKTTLDNQDGNFYGEFGALYDELAKIATLYNKVR |
| | | DYLSQKPFSTEKYKLNFGNPTLLNGWDLNKEKDNFGVILQKDGCYYLAL |
| | | LDKAHKKVFDNAPNTGKSVYQKMVYKLLPGSNKMLPKVFFAKSNLDYYN |
| | | PSAELLDKYAQGTHKKGDNFNLKDCHALIDFFKASINKHPEWQHFGFEF |
| | | SLTSSYQDLSDFYREVEPQGYQVKFVDIDADYIDELVEQGQLYLFQIYN |
| | | KDFSPKAHGKPNLHTLYFKALFSEDNLANPIYKLNGEAEIFYRKASLDM |
| | | NETTIHRAGEVLENKNPDNPKERQFVYDIIKDKRYTQDKFMLHVPITMN |
| | | FGVQGMTIKEFNKKVNQSIQQYDEVNVIGIDRGERHLLYLTVINSKGEI |
| | | LEQRSLNDIITTSANGTQMTTPYHKILDKREIERLNARVGWGEIETIKE |
| | | LKSGYLSHVVHQISQLMLKYNAIVVLEDLNFGFKRGRFKVEKQIYQNFE |
| | | NALIKKLNHLVLKDKADNEIGSYKNALQLTNNFTDLKSIGKQTGFLFYV |
| | | PAWNTSKIDPVTGFVDLLKPRYENIAQSQAFFDKEDKICYNADKGYFEF |
| | | HIDYAKFTDKAKNSRQIWTICSHGDKRYVYDKTANQNKGATIGINVNDE |
| | | LKSLFARYRINDKQPNLVMDICQNNDKEFHKSLTYLLKALLALRYSNAS |
| | | SDEDFILSPVANDKGVFFNSALADDTQPQNADANGAYHIALKGLWLLNE |
| | | LKNSDDLDKVKLAIDNQTWLNFAQNR |
| ART18 | 18 | MKYTDFTGIYPVSKTLRFELIPQGSTVENMKREGILNNDMHRADSYKEM |
| | | KKLIDEYHKVFIERCLSDFSLKYDDTGKHDSLEEYFFYYEQKRNDKTKK |
| | | IFEDIQVALRKQISKRFTGDTAFKRLFKKELIKEDLPSFVKNDPVKTEL |
| | | IKEFSDFTTYFQEFHKNRKNMYTSDAKSTAIAYRIINENLPKFIDNINA |
| | | FHIVAKVPEMQEHFKTIADELRSHLQVGDDIDKMENLQFFNKVLTQSQL |
| | | AVYNAVIGGKSEGNKKIQGINEYVNLYNQQHKKARLPMLKLLYKQILSD |
| | | RVAISWLQDEFDNDQDMLDTIEAFYNKLDSNETGVLGEGKLKQILMGLD |
| | | GYNLDGVFLRNDLQLSEVSQRLCGGWNIIKDAMISDLKRSVQKKKKETG |
| | | ADFEERVSKLFSAQNSFSIAYINQCLGQAGIRCKIQDYFACLGAKEGEN |
| | | EAETTPDIFDQIAEAYHGAAPILNARPSSHNLAQDIEKVKAIKALLDAL |
| | | KRLQRFVKPLLGRGDEGDKDSFFYGDEMPIWEVLDQLTPLYNKVRNRMT |
| | | RKPYSQEKIKLNFENSTLLNGWDLNKEHDNTSVILRREGLYYLGIMNKN |
| | | YNKIFDANNVETIGDCYEKMIYKLLPGPNKMLPKVFFSKSRVQEFSPSK |
| | | KILEIWESKSFKKGDNFNLDDCHALIDFYKDSIAKHPDWNKFNFKFSDT |
| | | QSYTNISDFYRDVNQQGYSLSFTKVSVDYVNRMVDEGKLYLFQIYNKDF |
| | | SPQSKGTPNMHTLYWRMLFDERNLHNVIYKLNGEAEVFYRKASLRCDRP |
| | | THPAHQPITCKNENDSKRVCVEDYDIIKNRRYTVDKFMFHVPITINYKC |
| | | TGSDNINQQVCDYLRSAGDDTHIIGIDRGERNLLYLVIIDQHGTIKEQF |
| | | SLNEIVNEYKGNTYCTNYHTLLEEKEAGNKKARQDWQTIESIKELKEGY |
| | | LSQVIHKISMLMQRYHAIVVLEDLNGSFMRSRQKVEKQVYQKFEHMLIN |
| | | KLNYLVNKQYDAAEPGGLLHALQLTSRMDSFKKLGKQSGELFYIPAWNT |
| | | SKIDPVTGFVNLEDTRYCNEAKAKEFFEKFDDISYNDERDWFEFSFDYR |
| | | HFTNKPTGTRTQWTLCTQGTRVRTFRNPEKSNHWDNEEFDLTQAFKDLF |
| | | NKYGIDIASGLKARIVNGQLTKETSAVKDFYESLLKLLKLTLQMRNSVT |
| | | GTDIDYLVSPVADKDGIFFDSRTCGSLLPANADANGAFNIARKGLMLLR |
| | | QIQQSSIDAEKIQLAPIKNEDWLEFAQEKPYL |
| ART19 | 19 | METFSGFTNLYPLSKTLRFRLIPVGETLKYFIGSGILEEDQHRAESYVK |
| | | VKAIIDDYHRAYIENSLSGFELPLESTGKENSLEEYYLYHNIRNKTEEI |
| | | QNLSSKVRTNLRKQVVAQLTKNEIFKRIDKKELIQSDLIDFVKNEPDAN |
| | | EKIALISEFRNFTVYFKGFHENRRNMYSDEEKSTSIAFRLIHENLPKFI |
| | | DNMEVFAKIQNTSISENFDAIQKELCPELVTLCEMEKLGYFNKTLSQKQ |
| | | IDAYNTVIGGKTTSEGKKIKGLNEYINLYNQQHKQEKLPKMKLLFKQIL |
| | | SDRESASWLPEKFENDSQVVGAIVNEWNTIHDTVLAEGGLKTIIASLGS |
| | | YGLEGIFLKNDLQLTDISQKATGSWGKISSEIKQKIEVMNPQKKKESYE |
| | | TYQERIDKIFKSYKSFSLAFINECLRGEYKIEDYFLKLGAVNSSSLQKE |
| | | NHFSHILNTYTDVKEVIGFYSESTDTKLIRDNGSIQKIKLFLDAVKDLQ |
| | | AYVKPLLGNGDETGKDERFYGDLIEYWSLLDLITPLYNMVRNYVTQKPY |
| | | SVDKIKINFQNPTLLNGWDLNKETDNTSVILRRDGKYYLAIMNNKSRKV |
| | | FLKYPSGTDRNCYEKMEYKLLPGANKMLPKVFFSKSRINEFMPNERLLS |
| | | NYEKGTHKKSGTCFSLDDCHTLIDFFKKSLDKHEDWKNFGFKFSDTSTY |
| | | EDMSGFYKEVENQGYKLSFKPIDATYVDQLVDEGKIFLFQIYNKDESEH |
| | | SKGTPNMHTLYWKMLFDETNLGDVVYKLNGEAEVFFRKASINVSHPTHP |
| | | ANIPIKKKNLKHKDEERILKYDLIKDKRYTVDQFQFHVPITMNFKADGN |
| | | GNINQKAIDYLRSASDTHIIGIDRGERNLLYLVVIDGNGKICEQFSLNE |
| | | IEVEYNGEKYSTNYHDLLNVKENERKQARQSWQSIANIKDLKEGYLSQV |
| | | IHKISELMVKYNAIVVLEDLNAGEMRGRQKVEKQVYQKFEKKLIEKLNY |
| | | LVFKKQSSDLPGGLMHAYQLANKFESFNTLGKQSGFLFYIPAWNTSKMD |
| | | PVTGFVNLEDVKYESVDKAKSFFSKFDSIRYNVERDMFEWKFNYGEFTK |
| | | KAEGTKTDWTVCSYGNRIITERNPDKNSQWDNKEINLTENIKLLFERFG |
| | | IDLSSNLKDEIMQRTEKEFFIELISLFKLVLQMRNSWTGTDIDYLVSPV |
| | | CNENGEFFDSRNVDETLPQNADANGAYNIARKGMILLDKIKKSNGEKKL |
| | | ALSITNREWLSFAQGCCKNG |
| ART20 | 20 | METFSGFTNLYPLSKTLRFRLIPVGETLKHFIDSGILEEDQHRAESYVK |
| | | VKAIIDDYHRAYIENSLSGFELPLESTGKENSLEEYYLYHNIRNKTEEI |
| | | QNLSSKVRTNLRKQVVVQLTKNEIFKRIDKKELIQSDLIDFVKNEPDAN |
| | | EKIALISEFRNFTVYFKGFHENRRNMYSDEEKSTSIAFRLIHENLPKFI |
| | | DNMEVFAKIQNTSISENFDAIQKELCPELVTLCEMFKLGYENKTLSQKQ |
| | | IDAYNTVIGGKTTSEGKKIKGLNEYINLYNQQHKQEKLPKMKLLFKQIL |
| | | SDRESASWLPEKFENDSQVVGAMVNFWNTIHDTVLAEGGLKTIIASLGS |
| | | YGLEGIFLKNDLQLTDISQKATGSWSKISSEIKQKIEVMNPQKKKESYE |
| | | SYQERIDKLFKSYKSFSLAFINECLRGEYKIEDYFLKLGAVNSSSLQKE |
| | | NHFSHILNAYTDVKEAIGFYSESTDTKLIQDNDSIQKIKQFLDAVKDLQ |
| | | AYVKPLLGNGDETGKDERFYGDLIEYWSLLDLITPLYNMVRNYVTQKPY |
| | | SVDKIKINFQNPTLLNGWDLNKETDNTSVILRRDGKYYLAIMNNKSRKV |
| | | FLKYPSGTDGNCYEKMEYKLLPGANKMLPKVFFSKSRINEFMPNERLLS |
| | | NYEKGTHKKSGICFSLDDCHTLIDFFKKSLDKHEDWKNFGFKFSDTSTY |
| | | EDMSGFYKEVENQGYKLSFKPIDATYVDQLVDEGKIFLFQIYNKDFSEH |
| | | SKGTPNMHTLYWKMLFDETNLGDVVYKLNGEAEVFFRKASINVSHPTHP |
| | | ANIPIKKKNLKHKDEERILKYDLIKDKRYTVDQFQFHVPITMNFKADGN |
| | | GNINQKAIDYLCSASDTHIIGIDRGERNLLYLVVIDGNGKICEQFSLNE |
| | | IEVEYNGEKYSTNYHDLLNVKENERKQARQSWQSIANIKDLKEGYLSQV |
| | | IHKISELMVKYNAIVVLEDLNAGEMRGRQKVEKQVYQKFEKKLIEKLNY |
| | | LVFKKQSSDLPGGLMHAYQLANKFESFNALGKQSGFLFYIPAWNTSKMD |
| | | PVTGFVNLFDVKYESVDKAKSFFSKFDSMRYNVERDMFEWKENYGEFTK |
| | | KAEGTKTDWTVCSYGNRIITFRNPDKNSQWDNKEINLTENIKLLFERFG |
| | | IDLSSNLKDEIMQRTEKEFFIELISLFKLVLQMRNSWTGTDIDYLVSPV |
| | | CNENGEFFDSRNVDETLPQNADANGAYNIARKGMILLDKIKKSNGEKKL |
| | | ALSITNREWLSFAQGCCKNG |
| ART21 | 21 | METFSGFTNLYPLSKTLRFRLIPVGETLKHFIGSGILEEDQHRAESYVK |
| | | VKAIIDDYHRTYIENSLSGFELPLESTGKENSLEEYYLYHNIRNKTEEI |
| | | QNLSSKVRTNLRKQVVTQLTKNEIFKRIDKKELIQSDLIDFVKNEPDAN |
| | | EKIALISEFRNFTVYFKGFHENRRNMYSDEEKSTSIAFRLIHENLPKFI |
| | | DNMEVFAKIQNTSISENFDAIQKELCPELVTLCEMEKLGYENKTLSQKQ |
| | | IDAYNTVIGGKTTSEGKKIKGLNEYINLYNQQHKQEKLPKMKLLFKQIL |
| | | SDRESASWLLEKFENDSQVVGAMVNFWNTIHDTVLAEGGLKTIIASLGS |
| | | YGLEGIFLKNDLQLTDISQKATGSWSKISSEIKQKIEAMNPQKKKESYE |
| | | SYQERIDKLFKSYKSFSLAFVNECLRGEYKIEDYFLKLGAVNSSLLQKE |
| | | NHFSHILNTYTDVKEVIGFYSESTDTKLIQDNDSIQKIKQFLDAVKDLQ |
| | | AYVKPLLGNSDETGKDERFYGDLIEYWSLLDLITPLYNMVRNYVTQKPY |
| | | SVDKIKINFQNPTLLNGWDLNKEMDNTSVILRRDGKYYLAIMNNKSRKV |
| | | FLKYPSGTDRNCYEKMEYKLLPGANKMLPKVFFSKSRINEFMPNERLLS |
| | | NYEKGTHKKSGTCFSLDDCHTLIDFFKKSLNKHEDWKNFGFKFSDTSTY |
| | | EDMSGFYKEVENQGYKLSFKPIDATYVDQLVDEGKIFLFQIYNKDFSEH |
| | | SKGTPNMHTLYWKMLFDETNLGDVVYKLNGEAEVFFRKASINVSHPTHP |
| | | ANIPIKKKNLKHKDEERILKYDLIKDKRYTVDQFQFHVPITMNFKANGN |
| | | GNINQKAIDYLRSASDTHIIGIDRGERNLLYLVVIDGNGKICEQFSLNE |
| | | IEVEYNGEKYSTNYHDLLNVKENERKQARQSWQSIANIKDLKEGYLSQV |
| | | IHKISELMVKYNAIVVLEDLNAGFMRGRQKVEKQVYQKFEKKLIEKLNY |
| | | LVFKKQSSDLPGGLMHAYQLANKFESENTLGKQSGFLFYIPAWNTSKMD |
| | | PVTGFVNLFDVKYESVDKAKSFFSKEDSIRYNVERDMFEWKENYDEFTK |
| | | KAEGTKTDWTVCSYGNRIITFRNPDKNSQWDNKEINLTENIKLLFERFG |
| | | IDLSSNLKDEIMERTEKEFFIELISLFKLVLQMRNSWTGTDIDYLVSPV |
| | | CNENGEFFDSRNVDETLPQNADANGAYNIARKGMILLDKIKKNNGEKKL |
| | | TLSITNREWLSFAQGCCKNG |
| ART22 | 22 | MLFQDFTHLYPLSKTVRFELKPIGKTLEHIHAKNFLSQDKTMADMYQKV |
| | | KAILDDYHRDFIADMMGEVKLTKLAEFCDVYLKFRKNPKDDGLQKQLKD |
| | | LQAVLRKEIVKPIGNGGKYKVGYDRLFGAKLFKDGKELGDLAKEVIAQE |
| | | SESSPKLPQIAHFEKFSTYFTGFHDNRKNMYSSDDKHTAIAYRLIHENL |
| | | PRFIDNLQILATIKQKHSALYDQIASELTASGLDVSLASHLGGYHKLLT |
| | | QEGITAYNRIIGEVNSYTNKHNQICHKSERIAKLRPLHKQILSDGMGVS |
| | | FLPSKFADDSEMCQAVNEFYRHYADVFAKVQSLEDREDDYQKDGIYVEH |
| | | KNLNELSKRAFGDFGELKRFLEEYYADVIDPEFNEKFAKTEPDSDEQKK |
| | | LAGEKDKFVKGVHSLASLEQVIEYYTAGYDDESVQADKLGQYFKHRLAG |
| | | VDNPIQKIHNSHSTIKGFLERERPAGERALPKIKSDKSPEMTQLRQLKE |
| | | LLDNALNVVHFAKLVSTETVLDTRSDKFYGEFRPLYVELAKITTLYNKV |
| | | RDYLSQKPFSTEKYKLNFGNPTLLNGWDLNKEKDNFGVILQKDGCYYLA |
| | | LLDKAHKKVFDNAPNTGKSVYQKMVYKQIANARRDLACLLIINGKVVRK |
| | | TKGLDDLREKYLPYDIYKIYQSESYKVLSPNFNHQDLVKYIDYNKILAS |
| | | GYFEYFDFRFKESSEYKSYKEFLDDVDNCGYKISFCNINADYIDELVEQ |
| | | GQLYLFQIYNKDFSPKAHGKPNLHTLYFKALFSEDNLANPIYKLNGEAQ |
| | | IFYRKASLDMNETTIHRAGEVLENKNPDNPKQRQFVYDIIKDKRYTQDK |
| | | FMLHVPITMNFGVQGMTIEGENKKVNQSIQQYDDVNVIGIDRGERHLLY |
| | | LTVINSKGEILEQRSLNDIITTSANGTQMTTPYHKILNKKKEGRLQARK |
| | | DWGEIETIKELKAGYLSHVVHQISQLMLKYNAIVVLEDLNFGFKRGRLK |
| | | VENQVYQNFENALIKKLNHLVLKDKTDDEIGSYKNALQLTNNFTDLKSI |
| | | GKQTGFLFYVPARNTSKIDPETGFVDLLKPRYENITQSQAFFGKEDKIC |
| | | YNTDKGYFEFHIDYAKFTDEAKNSRQTWVICSHGDKRYVYNKTANQNKG |
| | | ATKGINVNDELKSLFACHHINDKQPNLVMDICQNNDKEFHKSLMYLLKA |
| | | LLALRYSNANSDEDFILSPVANDEGVFFNSALADDTQPQNADANGAYHI |
| | | ALKGLWVLEQIKNSDDLDKVDLEIKDDEWRNFAQNR |
| ART23 | 23 | MGKNQNFQEFIGVSPLQKTLRNELIPTETTKKNITQLDLLTEDEIRAQN |
| | | REKLKEMMDDYYRDVIDSTLHAGIAVDWSYLFSCMRNHLRENSKESKRE |
| | | LERTQDSIRSQIYNKFAERADEKDMFGASIITKLLPTYIKQNPEYSERY |
| | | DESMEILKLYGKFTTSLTDYFETRKNIFSKEKISSAVGYRIVEENAEIF |
| | | LQNQNAYDRICKIAGLDLHGLDNEITAYVDGKTLKEVCSDEGFAKAITQ |
| | | EGIDRYNEAIGAVNQYMNLLCQKNKALKPGQFKMKRLHKQILCKGTTSF |
| | | DIPKKFENDKQVYDAVNSFTEIVMKNNDLKRLLNITQNVNDYDMNKIYV |
| | | AADAYSTISQFISKKWNLIEECLLDYYSDNLPGKGNAKENKVKKAVKEE |
| | | TYRSVSQLNELIEKYYVEKTGQSVWKVESYISRLAETITLELCHEIEND |
| | | EKHNLIEDDDKISKIKELLDMYMDAFHIIKVERVNEVLNFDETFYSEMD |
| | | EIYQDMQEIVPLYNHVRNYVTQKPYKQEKYRLYENTPTLANGWSKNKEY |
| | | DNNAIILMRDDKYYLGILNAKKKPSKQTMAGKEDCLEHAYAKMNYYLLP |
| | | GANKMLPKVFLSKKGIQDYHPSSYIVEGYNEKKHIKGSKNFDIRFCRDL |
| | | IDYFKECIKKHPDWNKENFEFSATETYEDISVFYREVEKQGYRVEWTYI |
| | | NSEDIQKLEEDGQLFLFQIYNKDFAVGSTGKPNLHTLYLKNLFSEENLR |
| | | DIVLKLNGEAEIFFRKSSVQKPVIHKCGSILVNRTYEITESGTTRVQSI |
| | | PESEYMELYRYENSEKQIELSDEAKKYLDKVQCNKAKTDIVKDYRYTMD |
| | | KFFIHLPITINFKVDKGNNVNAIAQQYIAEQEDLHVIGIDRGERNLIYV |
| | | SVIDMYGRILEQKSFNLVEQVSSQGTKRYYDYKEKLQNREEERDKARKS |
| | | WKTIGKIKELKEGYLSSVIHEIAQMVVKYNAIIAMEDLNYGFKRGRFKV |
| | | ERQVYQKFETMLISKLNYLADKSQAVDEPGGILRGYQMTYVPDNIKNVG |
| | | RQCGIIFYVPAAYTSKIDPTTGFINAFKRDVVSTNDAKENFLMKEDSIQ |
| | | YDIEKGLFKFSFDYKNFATHKLTLAKTKWDVYINGTRIQNMKVEGHWLS |
| | | MEVELTTKMKELLDDSHIPYEEGQNILDDLREMKDITTIVNGILEIFWL |
| | | TVQLRNSRIDNPDYDRIISPVLNNDGEFFDSDEYNSYIDAQKAPLPIDA |
| | | DANGAFCIALKGMYTANQIKENWVEGEKLPADCLKIEHASWLAFMQGER |
| | | G |
| ART24 | 24 | MNTSLFSSFTRQYPVTKTLRFELKPMGATLGHIQQKGFLHKDEELAKIY |
| | | KKIKELLDEYHRAFIADTLGDAQLVGLDDFYADYQALKQDSKNSHLKDK |
| | | LTKTQDNLRKQITKNFEKTPQLKERYKRLFTKELFKAGKDKGDLEKWLI |
| | | NHDSEPNKAEKISWIHQFENFTTYFQGFYENRKNMYSDEVKHTAIAYRL |
| | | IHENLPRFVDNIQVLSKIKSDYPDLYHELNHLDSRTIDFADFKEDDMLQ |
| | | MDFYHHLLIQSGITAYNTLLGGKVLEGGKKLQGINELINLYGQKHKIKI |
| | | AKLKPLHKQILSDGQSVSFLPKKFDNDYELCQTVNHFYREYVAIFDELV |
| | | VLFQKFYDYDKDNIYINHQQLNQLSHELFADERLLSRALDFYYCQIIDG |
| | | DENNKINNAKSQNAKEKLLKEKERYTKSNHSINELQKAINHYASHHEDT |
| | | EVKVISDYFSATNIRNMIDGIHHHFSTIKGFLEKDNNQGESYLPKQKNS |
| | | NDVKNLKLFLDGVLRLIHFIKPLALKSDDTLEKEEHFYGEFMPLYDKLV |
| | | MFTLLYNKVRDYISQKPYNDEKIKLNFGNSTLLNGWDVNKEKDNFGVIL |
| | | CKEGLYYLAILDKSHKKVFDNAPKATSSHTYQKMVYKLLPGPNKMLPKV |
| | | FFAKSNIGYYQPSAQLLENYEKGTHKKGSNFSLTDCHHLIDFFKSSIAK |
| | | HPEWKEFGFRFSDTHTYQDLSDFYKEIEPQSYKVKFIDIDADYIDDLVE |
| | | KGQLYLFQLYNKDFSKQSYGKPNLHTLYFKSLFSDDNLKNPIYKLNGEA |
| | | EIFYRRASLSVSDTTIHQAGEILTPKNPNNTHNRTLSYDVIKNKRYTTD |
| | | KFFLHIPITMNFGIENTGFKAFNHQVNTTLKNADKKDVHIIGIDRGERH |
| | | LLYVSVIDGDGRIVEQRTLNDIVSISNNGMSMSTPYHQILDNREKERLA |
| | | ARTDWGDIKNIKELKAGYLSHVVHEVVQMMLKYNAMIVLEDLNFGFKHG |
| | | RFKVEKQVYQNFENALIKKLNYLVLKNADNHQLGSVRKALQLTNNFTDI |
| | | KSIGKQTGFIFYVPAWNTSKIDPTTGFVDLLKPRYENMAQAQSFISRFK |
| | | KIAYNHQLDYFEFEFDYADFYQKTIDKKRIWTLCTYGDVRYYYDHKTKE |
| | | TKTVNITKELKSLLDKHDLSYQNGHNLVDELANSHDKSLLSGVMYLLKV |
| | | LLALRYSHAQKNEDFILSPVMNKDGVFFDSRFADDVLPNNADANGAYHI |
| | | ALKGLWVLNQIQSADNMDKIDLSISNEQWLHFTQSR |
| ART25 | 25 | MVGNKISNSFDSFTGINALSKTLRNELIPSDYTKRHIAESDFIAADTNK |
| | | NEDQYVAKEMMDDYYRDFISKVLDNLHDIEWKNLFELMHKAKIDKSDAT |
| | | SKELIKIQDMLRKKIGKKFSQDPEYKVMLSAGMITKILPKYILEKYETD |
| | | REDRLEAIKRFYGFTVYFKEFWASRQNVESDKAIASSISYRIIHENAKI |
| | | YMDNLDAYNRIKQIACEEIEKIEEEAYDFLQGDQLDVVYTEEAYGRFIS |
| | | QSGIDLYNNICGVINAHMNLYCQSKKCSRSKFKMQKLHKQILCKAETGF |
| | | EIPLGFQDDAQVINAINSFNALIKEKNIISRLRTIGKSISLYDVNKIYI |
| | | SSKAFENVSVYIDHKWDVIASSLYKYFSEIVKGNKDNREEKIQKEIKKV |
| | | KSCSLGDLQRLVNSYYKIDSTCLEHEVTEFVTKIIDEIDNFQITDEKEN |
| | | DKISLIQNEQIVMDIKTYLDKYMSIYHWMKSFVIDELVDKDMEFYSELD |
| | | ELNEDMSEIVNLYNKVRNYVTQKPYSQEKIKLNFGSPTLADGWSKSKEF |
| | | DNNAIILIRDEKIYLAIFNPRNKPAKTVISGHDVCNSETDYKKMNYYLL |
| | | PGASKTLPHVFIKSRLWNESHGIPDEILRGYELGKHLKSSVNEDVEFCW |
| | | KLIDYYKECISCYPNYKAYNFKFADTESYNDISEFYREVECQGYKIDWT |
| | | YISSEDVEQLDRDGQIYLFQIYNKDFAPNSKGMDNLHTKYLKNIFSEDN |
| | | LKNIVIKLNGEAELFYRKSSVKKKVEHKKGTILVNKTYKVEDNTENSKE |
| | | KRVIIESVPDDCYMELVDYWRNGGIGILSDKAVQYKDKVSHYEATMDIV |
| | | KDRRYTVDKFFIHLPITINFKADGRININEKVLKYIAENDELHVIGIDR |
| | | GERNLLYVSVINKKGKIVEQKSENMIESYETVTNIVRRYNYKDKLVNKE |
| | | SARTDARKNWKEIGKIKEIKEGYLSQVIHEISKMVLKYNAIIVMEDLNY |
| | | GFKRGRFRVERQVYQKFENMLISKLAYLVDKSRKADEPGGVLRGYQLTY |
| | | IPDSLEKLGSQCGIIFYVPAAYTSKIDPLTGFVNVENFREYSNFETKLD |
| | | FVRSLDSIRYDTEKKLFSISFDYDNFKTHNTTLAKTKWVIYLRGERIKK |
| | | EHTSYGWKDDVWNVESRIKDLFDSSHMKYDDGHNLIEDILELESSVQKK |
| | | LINELIEIIRLTVQLRNSKSERYDRTEAEYDRIVSPVMDENGRFYDSEN |
| | | YIFNEETELPKDADANGAYCIALKGLYNVIAIKNNWKEGEKENRKLLSL |
| | | NNYNWFDFIQNRRF |
| ART26 | 26 | MVGNKISNSFDSFTGINALSKTLRNELIPSDYTKRHIAESDFIAADTNK |
| | | NEDQYVAKEMMDDYYRDFISKVLDNLHDIEWKNLFELMHKAKIDKSDAT |
| | | SKELIKIQDMLRKKIGKKESQDPEYKVMLSAGMITKILPKYILEKYETD |
| | | REDRLEAIKRFYGFTVYFKEFWASRQNVESDKAIASSISYRIIHENAKI |
| | | YMDNLDAYNRIKQIACEEIEKIEEEAYDFLQGDQLDVVYTEEAYGRFIS |
| | | QSGIDLYNNICGVINAHMNLYCQSKKCSRSKFKMQKLHKQILCKAETGF |
| | | EIPLGFQDDAQVINAINSFNALIKEKNIISRLRTIGKSISLYDVNKIYI |
| | | SSKAFENVSVYIDHKWDVIASSLYKYFSEIVKGNKDNREEKIQKEIKKV |
| | | KSCSLGDLQRLVNSYYKIDSTCLEHEVTEFVTKIIDEIDNFQITDEKEN |
| | | DKISLIQNEQIVMDIKTYLDKYMSIYHWMKSFVIDELVDKDMEFYSELD |
| | | ELNEDMSEIVNLYNKVRNYVTQKPYSQEKIKLNFGSPTLADGWSKSKEF |
| | | DNNAIILIRDEKIYLAIFNPRNKPAKTVISGHDVCNSETDYKKMNYYLL |
| | | PGASKTLPHVFIKSRLWNESHGIPDEILRGYELGKHLKSSVNEDVEFCW |
| | | KLIDYYKECISCYPNYKAYNFKFADTESYNDISEFYREVECQGYKIDWT |
| | | YISSEDVEQLDRDGQIYLFQIYNKDFAPNSKGMDNLHTKYLKNIFSEDN |
| | | LKNIVIKLNGEAELFYRKSSVKKKVEHKKGTILVNKTYKVEDNTENSKE |
| | | KRVIIESVPDDCYMELVDYWRNGGIGILSDKAVQYKDKVSHYEATMDIV |
| | | KDRRYTVDKFFIHLPITINFKADGRININEKVLKYIAENDELHVIGIDR |
| | | GERNLLYVSVINKKGKIVEQKSENMIESYETVINIVRRYNYKDKLVNKE |
| | | SARTDARKNWKEIGKIKEIKEGYLSQVIHEISKMVLKYNAIIVMEDLNY |
| | | GFKRGRFRVERQVYQKFENMLISKLAYLVDKSRKADEPGGVLRGYQLTY |
| | | IPDSLEKLGSQCGIIFYVPAAYTSKIDPLTGFVNVENFREYSNFETKLD |
| | | FVRSLDSIRYDTEKKLFSISEDYDNFKTHNTTLAKTKWVIYLRGERIKK |
| | | EHTSYGWKDDVWNVESRIKDLFDSSHMKYDDGHNLIEDILELESSVQKK |
| | | LINELIEIIRLTVQLRNSKSERYDRTEAEYDRIVSPVMDENGRFYDSEN |
| | | YIFNEETELPKDADANGAYCIALKGLYNVIAIKNNWKEGEKFNRKLLSL |
| | | NNYNWFDFIQNRRFQIYLFQIYNKDFAPNSKGMDNLHTKYLKNIFSEDN |
| | | LKNIVIKLNGEAELFYRKSSVKKKVEHKKGTILVNKTYKVEDNTENSKE |
| | | KRVIIESVPDDCYMELVDYWRNGGIGILSDKAVQYKDKVSHYEATMDIV |
| | | KDRRYTVDKFFIHLPITINFKADGRININEKVLKYIAENDELHVIGIDR |
| | | GERNLLYVSVINKKGKIVEQKSENMIESYETVTNIVRRYNYKDKLVNKE |
| | | SARTDARKNWKEIGKIKEIKEGYLSQVIHEISKMVLKYNAIIVMEDLNY |
| | | GFKRGRFRVERQVYQKFENMLISKLAYLVDKSRKADEPGGVLRGYQLTY |
| | | IPDSLEKLGSQCGIIFYVPAAYTSKIDPLTGFVNVENFREYSNFETKLD |
| | | FVRSLDSIRYDTEKRLFSISFDYDNFKTHNTTLAKTKWVIYLRGERIKK |
| | | EHTSYGWKDDVWNVESRIKDLFDSSHMKYDDGHNLIEDILELESSVQKK |
| | | LINELIEIIRLTVQLRNSKSERYDRTEAEYDRIVSPVMDEKGRFYDSEN |
| | | YIFNEETELPKDADANGAYCIALKGLYNVIAIKNNWKEGEKFNRKLLSL |
| | | NNYNWFDFIQNRRF |
| ART27 | 27 | MQEHKKISHLTHRNSVQKTIRMQLNPVGKTMDYFQAKQILENDEKLKED |
| | | YQKIKEIADRFYRNLNEDVLSKTGLDKLKDYAEIYYHCNTDADRKRLDE |
| | | CASELRKEIVKNFKNRDEYNKLENKKMIEIVLPQHLKNEDEKEVVASFK |
| | | NFTTYFTGFFTNRKNMYSDGEESTAIAYRCINENLPKHLDNVKAFEKAI |
| | | SKLSKNAVDDLDTTYSGLCGTNLYDVFTVDYFNFLLPQSGITEYNKIIG |
| | | GYTTSDGTKVKGINEYINLYNQQVSKRYKIPNLKILYKQILSESEKVSF |
| | | IPPKFEDDNELLSAVSEFYANDETFDGMPLKKAIDETKLLFGNLDNSSL |
| | | NGIYIQNDRSVINLSNSMFGSWSVIEDLWNKNYDSVNSNSRIKDIQKRE |
| | | DKRKKAYKAEKKLSLSFLQVLISNSENDEIREKSIVDYYKTSLMQLTDN |
| | | LSDKYKEAAPLFNESYANEKGLKNDDKSISLIKNFLDAIKEIEKFIKPL |
| | | SETNITGEKNDLFYSQFTPLLDNISRIDILYDKVRNYVTQKPFSTDKIK |
| | | LNFGNSQLLNGWDRNKEKDCGAVWLCKDEKYYLAIIDKSNNSILENIDE |
| | | QDCDESDCYEKIIYKLLPGPNKMLPKVFFSEKCKKLLSPSDEILKIRKN |
| | | GTFKKGDKFSLDDCHKLIDFYKESFKKYPNWLIYNFKFKKTNEYNDISE |
| | | FYNDVASQGYNISKMKIPTSFIDKLVDEGKIYLFQLYNKDESPHSKGTP |
| | | NLHTLYFKMLFDERNLEDVVYKLNGEAEMFYRPASIKYDKPTHPKNTPI |
| | | KNKNTLNDKRASTFPYDLIKDKRYTKWQFSLHFPITMNFKAPDRAMIND |
| | | DVRNLLKSCNNNFIIGIDRGERNLLYVSIIDSNGAIIYQHSLNIIGNKF |
| | | KGKTYETNYREKLETREKERTEQRRNWKAIESIKELKEGYISQAVHVIC |
| | | QLVVKYDAIIVMEKLTDGFKRGRTKFEKQVYQKFEKMLIDKLNYYVDKK |
| | | LDPDEEGGLLHAYQLTNKLESFDKLGMQSGFIFYVRPDFTSKIDPVTGF |
| | | VNLLYPRYENIDKAKDMISREDDIRYNAGEDFFEFDIDYDKFPKTASDY |
| | | RKKWTICTNGERIEAFRNPASNNEWSYRTIILAEKFKELFDNNSINYRD |
| | | SDNLKAEILSQTKGKFFEDFFKLLRLTLQMRNSNPETGEDRILSPVKDK |
| | | NGNFYDSSKYDEKSNLPCDADANGAYNIARKGLWIVEQFKKSDNVSTVE |
| | | PVIHNDKWLKFVQENDMANN |
| ART28 | 28 | MKNLANFTNLYSLQKTLRFELKPIGKTLDWIIKKDLLKQDEILAEDYKI |
| | | VKKIIDRYHKDFIDLAFESAYLQKKSSDSFTAIMEASIQSYSELYFIKE |
| | | KSDRDKKAMEEISGIMRKEIVECFTGKYSEVVKKKFGNLFKKELIKEDL |
| | | LNFCEPDELPIIQKFADETTYFTGFHENRENMYSNEEKATAIANRLIRE |
| | | NLPRYLDNLRIIRSIQGRYKDFGWKDLESNLKRIDKNLQYSDELTENGF |
| | | VYTFSQKGIDRYNLILGGQSVESGEKIQGLNELINLYRQKNQLDRRQLP |
| | | NLKELYKQILSDRTRHSFVPEKFSSDKALLRSLLDFHKEVIQNKNLFEE |
| | | KQVSLLQAIRETLTDLKSFDLDRIYLINDTSLTQISNFVFGDWSKVKTI |
| | | LAIYFDENIANPKDRQRQSNSYLKAKENWLKKNYYSIHELNEAISVYGK |
| | | HSDEELPNTKIEDYFSGLQTKDETKKPIDVLDAIVSKYADLESLLTKEY |
| | | PEDKNLKSDKGSIEKIKNYLDSIKLLQNFLKPLKPKKVQDEKDLGFYND |
| | | LELYLESLESANSLYNKVRNYLTGKEYSDEKIKLNFKNSTLLDGWDENK |
| | | ETSNLSVIFRDTNNYYLGILDKQNNRIFESIPEIQSGEETIQKMVYKLL |
| | | PGANNMLPKVFFSEKGLLKFNPSDEITSLYSEGRFKKGDKESINSLHTL |
| | | IDFYKKSLAVHEDWSVENFKFDETSHYEDISQFYRQVESQGYKITFKPI |
| | | SKKYIDTLVEDGKLYLFQIYNKDFSQNKKGGGKPNLHTIYFKSLFEKEN |
| | | LKDVIVKLNGQAEVFFRKKSIHYDENITRYGHHSELLKGRFSYPILKDK |
| | | RFTEDKFQFHFPITLNFKSGEIKQFNARVNSYLKHNKDVKIIGIDRGER |
| | | HLLYLSLIDQDGKILRQESLNLIKNDQNFKAINYQEKLHKKEIERDQAR |
| | | KSWGSIENIKELKEGYLSQVVHTISKLMVEHNAIVVLEDLNFGFKRGRQ |
| | | KVERQVYQKFEKMLIEKLNFLVFKDKEMDEPGGILKAYQLTDNFVSFEK |
| | | MGKQTGFVFYVPAWNTSKIDPKTGFVNFLHLNYENVNQAKELIGKEDQI |
| | | RYNQDRDWFEFQVTTDQFFTKENAPDTRIWIICSTPTKRFYSKRTVNGS |
| | | VSTIEIDVNQKLKELFNDCNYQDGEDLVDRILEKDSKDFFSKLIAYLRI |
| | | LTSLRQNNGEQGFEERDFILSPVVGSDGKFFNSLDASSQEPKDADANGA |
| | | YHIALKGLMNLHVINETDDESLGKPSWKISNKDWLNFVWQRPSLKA |
| ART29 | 29 | MQEHKKISHLTHRNSVQKTIRMQLNPVGKTMDYFQAKQILENDEKLKEN |
| | | YQKIKEIADRFYRNLNEDVLSKTRLDKLKDYTDIYYHCNTDADRKRLDE |
| | | CASELRKEIVKNFKNRDEYNKLENKKMIEIVLPKHLKNEDEKEVVTSFK |
| | | NFTTYFTGFFTNRKNMYSDGEESTAIAYRCINENLPKHLDNVKAFEKAI |
| | | SKLSKNAIDDLDTTYSGLCGTNLYDVFTVDYFNFLLPQSGITEYNKIIG |
| | | GYTTNDGTKVKGINEYINLYNQQVSKRDKIPNLKILYKQILSESEKVSF |
| | | IPPKFEDDNELLSAVSEFYANDETFDGMPLKKAIDETKLLFGNLDNPSL |
| | | NGIYIQNDRSVTNLSNSMFGSWSVIEDLWNKNYDSVNSNSRIKDIQKRE |
| | | DKRKKAYKAEKKLSLSFLQVLISNSENDEIREKSIVDYYKTSLMQLTDN |
| | | LSDKYNEAAPLLNENYSNEKGLKNDDKSISLIKNFLDAIKEIEKFIKPL |
| | | SETNITGEKNDLFYSQFTPLLDNISRIDILYDKVRNYVTQKPFSTDKIK |
| | | LNFGNSQLLNGWDRNKEKDCGAVWLCKDEKYYLAIIDKSNNSILENIDF |
| | | QDCDESDCYEKIIYKLLPGPNKMLPKVFFSEKCKKLLSPSDEILKIYKS |
| | | GTFKTGDKFSLDDCHKLIDFYKESFKKYPNWLIYNFKFKKTNEYNDIRE |
| | | FYNDVALQGYNISKMKIPTSFIDKLVDEGKIYLFQLYNKDFSPHSKGTP |
| | | NLHTLYFKMLFDERNLEDVVYRLNGEAEMFYRPASIKYDKPTHPKNTPI |
| | | KNKNTLNDKKTSTFPYDLIKDKRYTKWQFSLHFPITMNFKAPDKAMIND |
| | | DVRNLLKSCNNNFIIGIDRGERNLLYVSVIDSNGAIIYQHSLNIIGNKF |
| | | KEKTYETNYREKLATREKERTEQRRNWKAIESIKELKEGYISQAVHVIC |
| | | QLVVKYDAIIVMEKLTDGFKRGRTKFEKQVYQKFEKMLIDKLNYYVDKK |
| | | LDPDEEGGLLHAYQLTNKLESFDKLGMQSGFIFYVRPDFTSKIDPVTGF |
| | | VNLLYPQYENIDKAKDMISREDEIRYNAGEDFFEFDIDYDEFPKTASDY |
| | | RKKWTICTNGERIEAFRNPANNNEWSYRTIILAEKFKELFDNNSINYRD |
| | | SDDLKAEILSQTKGKFFEDFFKLLRLTLQMRNSNPETGEDRILSPVKDK |
| | | NGNFYDSSKYDEKSKLPCDADANGAYNIARKGLWIVEQFKKADNVSTVE |
| | | PVIHNDQWLKFVQENDMANN |
| ART30 | 30 | MQEHKKISHLTHRNSVQKTIRMQLNPVGKTMDYFQAKQILENDEKLKED |
| | | YQKIKEIADRFYRNLNEDVLSKTGLDKLKDYADIYYHCNTDADRKRLNE |
| | | CASELRKEIVKNFKNRDEYNKLENKKMIEIVLPKHLKNEDEKEVVASFK |
| | | NFTTYFTGFFTNRKNMYSDGEESTAIAYRCINENLPKHLDNVKVFEKAI |
| | | SKLSKNAIDDLGATYSGLCGTNLYDVFTVDYFNFLLPQSGITEYNKIIG |
| | | GYTTSDGTKVKGINEYINLYNQQVSKRDKIPNLKILYKQILSESEKVSF |
| | | IPPKFEDDNELLSAVSEFYANDETFDGMPLKKAIDETKLLFGNLDNSSL |
| | | NGIYIQNDRSVINLSNSMFGSWSVIEDLWNKNYDSVNSNSRIKDIQKRE |
| | | DKRKKAYKAEKKLSLSFLQVLISNSENDEIREKSIVDYYKTSLMQLTDN |
| | | LSDKYKEAAPLFSENYDNEKGLKNDDKSISLIKNFLDAIKEIEKFIKPL |
| | | SETNITGEKNDLFYSQFTPLLDNISRIDILYDKVRNYVTQKPFSTDKIK |
| | | LNFGNSQLLNGWDKDKEREYGAVLLCKDEKYYLAIIDKSNNSILENIDF |
| | | QDCNESDYYEKIVYKLLTKINGNLPRVFFSEKRKKLLSPSDEILKIYKS |
| | | GTFKKGDKFSLDDCHKLIDFYKESFKKYPNWLIYNFKFKNTNEYNDISE |
| | | FYNDVASQGYNISKMKIPTTFIDKLVDEGKIYLFQLYNKDESPHSKGTP |
| | | NLHTLYFKMLFDERNLEDVVYKLNGEAEMFYRPASIKYDKPTHPKNTPI |
| | | KNKNTLNDKKASTFPYDLIKDKRYTKWQFSLHFPITMNFKAPDKAMIND |
| | | DVRNLLKSCNNNFIIGIDRGERNLLYVSVIDSNGAIIYQHSLNIIGNKE |
| | | KGKTYETNYREKLATREKDRTEQRRNWKAIESIKELKEGYISQAVHVIC |
| | | QLVVKYDAIIVMEKLTDGFKRGRTKFEKQVYQKFEKMLIDKLNYYVDKK |
| | | LDPDEEGGLLHAYQLTNKLESFDKLGTQSGFIFYVRPDFTSKIDPVTGF |
| | | VNLLYPRYENIDKAKDMISREDDIRYNAGEDFFEFDIDYDKFPKTASDY |
| | | RKKWTICTNGERIEAFRNPANNNEWSYRTIILAEKFKELFDNNSINYRD |
| | | SDDLKAEILSQTKGKFFEDFFKLLRLTLQMRNSNPETGEDRILSPVKDK |
| | | NGNFYDSSKYDEKSKLPCDADANGAYNIARKGLWIVEQFKKADNVSTVE |
| | | PVIHNDKWLKFVQENDMANN |
| ART31 | 31 | MQERKKISHLTHRNSVKKTIRMQLNPVGKTMDYFQAKQILENDEKLKEN |
| | | YQKIKEIADRFYRNLNEDVLSKTGLDKLKDYAEIYYHCNTDADRKRLNK |
| | | CASELRKEIVKNFKNRDEYNKLEDKRMIEIVLPKHLKNEDEKEVVASFK |
| | | NFTTYFTGFFTNRKNMYSDGEESTAIAYRCINENLPKHLDNVKAFEKAI |
| | | SKLSKNAIDDLDAYSGLCGTNLYDVFTVDYFNFLLPQSGITEYNKIIGG |
| | | YTTNDGTKVKGINEYINLYNQQVSKRDKIPNLQILYKQILSESEKVSFI |
| | | PPKFEDDNELLSAVSEFYANDETFDGMPLKKAIDETKLLFGNLDNSSLN |
| | | GIYIQNDRSVTNLSNSMFGSWSVIEDLWNKNYDSVNSNSRIKDIQKRED |
| | | KRKKAYKAEKKLSLSFLQVLISNSENDEIRKKSIVDYYKTSLMQLTDNL |
| | | SDKYNEAAPLLNENYSNEKGLKNDDKSISLIKNFLDAIKEIEKFIKPLS |
| | | ETNITGEKNDLFYSQFTPLLDNISRIDILYDKVRNYVTQKPFSTDKIKL |
| | | NFGNYQLLNGWDKDKEREYGAVLLCKDEKYYLAIIDKSNNRILENIDFQ |
| | | DCDESDCYEKIIYKLLPTPNKMLPKVFFAKKHKKLLSPSDEILKIYKNG |
| | | TFKKGDKFSLDDCHKLIDFYKESFKKYPKWLIYNFKFKKINGYNDIREF |
| | | YNDVALQGYNISKMKIPTSFIDKLVDEGKIYLFQLYNKDFSPHSKGTPN |
| | | LHTLYFKMLFDERNLEDVVYRLNGEAEMFYRPASIKYDKPTHPKNTPIK |
| | | NKNTLNDKRASTFPYDLIKDKRYTKWQFSLHFPITMNFKDPDKAMINDD |
| | | VRNLLKSCNNNFIIGIDRGERNLLYVSVINSNGAIIYQHSLNIIGNKFK |
| | | GKTYETNYREKLATREKDRTEQRRNWKAIESIKELKEGYISQAVHVICQ |
| | | LVVKYDAIIVMEKLTDGFKRGRTKFEKQVYQKFEKMLIDKLNYYVDKKL |
| | | DPDEEGGLLHAYQLTNKLESFDKLGTQSGFIFYVRPDFTSKIDPVTGFV |
| | | NLLYPRYEKIDKAKDMISREDDIRYNAGEDFFEFDIDYDKFPKTASDYR |
| | | KKWTICTNGERIEAFRNPANNNEWSYRTIILAEKFKELFDNNSINYRDS |
| | | DDLKAEILSQTKGKFFEDFFKLLRLTLQMRNSNPETGEDRILSPVKDKN |
| | | GNFYDSSKYDEKSKLPCDADANGAYNIARKGLWIVEQFKKADNVSTVEP |
| | | VIHNDKWLKFVQENDMANN |
| ART32 | 32 | KTGLDKLKDYAEIYYHCNTDADRKRLNKCASELRKEIVKNFKNRDEYNK |
| | | LFDKRMIEIVLPKHLKNEDEKEVVASFKNFTTYFTGFFTNRKNMYSDGE |
| | | ESTAIAYRCINENLPKHLDNVKAFEKAISKLSKNAIDDLDATYSGLCGT |
| | | NLYDVFTVDYFNFLLPQSGITEYNKIIGGYTTSDGTKVKGINEYINLYN |
| | | QQVSKRDKIPNLQILYKQILSESEKVSFIPPKFEDDNELLSAVSEFYAN |
| | | DETFDEMPLKKAIDETKLLFGNLDNSSLNGIYIQNDRSVINLSNSMFGS |
| | | WSVIEDLWNKNYDSVNSNSRIKDIQKREDKRKKAYKAEKKLSLSFLQVL |
| | | ISNSENNEIREKSIVDYYKTSLMQLTDNLSDKYNEVAPLLNENYSNEKG |
| | | LKNDDKSISLIKNFLDAIKEIEKFIKPLSETNITGEKNDLFYSQFTPLL |
| | | DNISRIDILYDKVRNYVTQKPFSTDKIKLNFGNYQLLNGWDKDKEREYG |
| | | AVLLCRDEKYYLAIIDKSNNRILENIDFQDCDESDCYEKIIYKLLPTPN |
| | | KMLPKVFFAKKHKKLLSPSDEILKIRKNGTFKKGDKFSLDDCHKLIDFY |
| | | KESFKKYPNWLIYNFKFKKTNEYNDIREFYNDVALQGYNISKMKIPTSF |
| | | IDKLVDEGKIYLFQLYNKDFSPHSKGTPNLHTLYFKMLEDERNLEDVVY |
| | | KLNGEAKMFYRPASIKYDKPTHPKNTPIKNKNTLNDKKASTFPYDLIKD |
| | | KRYTKWQFSLHESITMNFKAPDKAMINDDVRNLLKSCNNNFIIGIDRGE |
| | | RNLLYVSVIDSNGAIIYQHSLNIIGNKEKGKTYETNYREKLATREKERT |
| | | EQRRNWKAIESIKELKEGYISQAVHVICQLVVKYDAIIVMEKLTDGFKR |
| | | GRTKFEKQVYQKFEKMLIDKLNYYVDKKLDPDEEGGLLHAYQLTNKLES |
| | | FDKLGTQSGFIFYVRPDFTSKIDPVTGFVNLLYPRYENIDKAKDMISRF |
| | | DDIRYNAGEDFFEFDIDYDKFPKTASDYRKKWTICTNGERIEAFRNPAN |
| | | NNEWSYRTIILAEKFKELFDNNSINYRDSDDLKAEILSQTKGKFFEDFF |
| | | KLLRLTLQMRNSNPETGEDRILSPVKDKNGNFYDSSKYDEKSKLPCDAD |
| | | ANGAYNIARKGLWIVEQFKKSDNVSTVEPVIHNDKWLKFVQENDMANN |
| ART33 | 33 | MSININKFSDECRKIDFFTDLYNIQKTLRFSLIPIGATADNFEFKGRLS |
| | | KEKDLLDSAKRIKEYISKYLADESDICLSQPVKLKHLDEYYELYITKDR |
| | | DEQKFKSVEEKLRKELADLLKEILKRLNKKILSDYLPEYLEDDEKALED |
| | | IANLSSFSTYFNSYYDNCKNMYTDKEQSTAIPYRCINDNLPKFIDNMKA |
| | | YEKALEELKPSDLEELRNNFKGVYDTTVDDMFTLDYENCVLSQSGIDSY |
| | | NAIIGNDKVKGINEYINLHNQTAEQGHKVPNLKRLYKQIGSQKKTISFL |
| | | PSKFESDNELLKAVYDFYNTGDAEKNFTALKDTITEFEKIFDNLSEYNL |
| | | DGVFVRNDISLTNLSQSMENDWSVFRNLWNDQYDKVNNPEKAKDIDKYN |
| | | DKRHKVYKKSESFSINQLQELIATTLEEDINSKKITDYFSCDFHRVTTE |
| | | VENKYQLVKDLLSSDYPKNKNLKTSEEDVALIKDELDSVKSLESFVKIL |
| | | TGTGKESGKDELFYGSFTKWFDQLRYIDKLYDKVRNYITEKPYSLDKIK |
| | | LSFDNPQFLGGWQHSKETDYSAQLFMKDGLYYLGVMDKETKREFKTQYN |
| | | TPENDSDTMVKIEYNQIPNPGRVIQNLMLVDGKIVKKNGRKNADGVNAV |
| | | LEELKNQYLPENINRIRKTESYKTTSNNENKDDLKAYLEYYIARTKEYY |
| | | CKYNFVFKSADEYGSFNEFVDDVNNQAYQITKVKVSEKQLLSLVEQGKL |
| | | YLFKIYNKDFSEYSKGKKNLHTMYFQMLFDDRNLENLVYKLQGGAEMFY |
| | | RPASIKKDSEFKHDANVEIIKRTCEDKVNDKDNPTDDEKAKYYSKFDYD |
| | | IVKNKRFTKDQFSLHLTLAMNCNQPDHYWLNNDVRELLKKSNKNHIIGI |
| | | DRGERNLIYVTIINSDGVIVDQINFNIIENSYNGKKYKTDYQKKLNQRE |
| | | EDRQKARKTWKTIETIKELKDGYISQVVHQICKLIVQYDAIVVMENING |
| | | GFKRGRTKVEKQVYQKFETMLINKLNYYVDKGTDYKECGGLLKAYQLTN |
| | | KFETFERIGKQSGIIFYVDPYLTSKIDPVTGFANLLYPKYETIPKTHNF |
| | | ISNIDDIRYNQSEDYFEFDIDYDKFPQGSYNYRKKWTICSYGNRIKYYK |
| | | DSRNKTASVVVDITEKEKETFTNAGIDFVNDNIKEKLLLVNSKELLKSF |
| | | MDTLKLTVQLRNSEINSDVDYIISPIKDRNGNFYYSENYKKSNNEVPSQ |
| | | PQDGDANGAYNIARKGLMIINKLKKADDVINNELLKISKKEWLEFAQKG |
| | | DLGE |
| ART34 | 34 | MKATSIWDNFTRKYSVSKTLRFELRPVGKTEENIVKKEIIDAEWISGKN |
| | | IPKGTDADRARDYKIVKKLLNQLHILFINQALSSENVKEFEKEDKKSKT |
| | | FVAWSDLLATHFDNWIQYTRDKSNSTVLKSLEKSKKDLYSKLGKLLNSK |
| | | ANAWKAEFISYHKIKSPDNIKIRLSASNVQILFGNTSDPIQLLKYQIEL |
| | | DNIKFLKDDGSEYTTKELADLLSTFEKFGTYFSGFNQNRANVYDIDGEI |
| | | STSIAYRLFNQNIEFFFQNIKRWEQFTSSIGHKEAKENLKLVQWDIQSK |
| | | LKELDMEIVQPRFNLKFEKLLTPQSFIYLLNQEGIDAFNTVLGGIPAEV |
| | | KAEKKQGVNELINLTRQKLNEDKRKFPSLQIMYKQIMSERKINFIDQYE |
| | | DDVEMLKEIQEFSNDWNEKKKRHSASSKEIKESAIAYIQREFHETFDSL |
| | | EERATVKEDFYLSEKSIQNLSIDIFGGYNTIHNLWYTEVEGMLKSGERP |
| | | LTRVEKEKLKKQEYISFAQIERLISKHSQQYLDSTPKEANDRSLFKEKW |
| | | KKTFKNGFKVSEYTNLKLNELISEGETFQKIDQETGKETTIKIPGLFES |
| | | YENAILVESIKNQSLGTNKKESVPSIKEYLDSCLRLSKFIESFLVNSKD |
| | | LKEDQSLDGCSDFQNTLTQWLNEEFDVFILYNKVRNHVTKKPGNTDKIK |
| | | INFDNATLLDGWDVDKEAANFGFLLKKADNYYLGIADSSFNQDLKYFNE |
| | | GERLDEIEKNRKNLEKEESKNISKIDQEKVKKYKEVIDDLKAISNLNKG |
| | | RYSKAFYKQSKFTTLIPKCTTQLNEVIEHFKKEDTDYRIENKKFAKPFI |
| | | ITKEVFLLNNTVYDTATKKFTLKIGEDEDTKGLKKFQIGYYRATDDKKG |
| | | YESALRNWITFCIEFTKSYKSCLNYNYSSLKSVSEYKSLDEFYKDLNGI |
| | | GYTIDFVDISEEYINKKINEGKLYLFQIYNKDFSEKSKGKENLHTTYWK |
| | | LLFDSKNLEDVVIKLNGQAEVFFRPASIHEKEKITHEKNQEIQNKNPNA |
| | | VKKTSKFEYDIIKDNRFTKNKFLFHCPITLNFKADGNPYVNNEVQENIA |
| | | KNPNVNIIGIDRGEKHLLYFTVINQQGQILDAGSLNSIKSEYKDKNQQS |
| | | VSFETPYHKILDKKESERKEARESWQEIENIKELKAGYLSHVVHQLSNL |
| | | IVKYNAIVVLEDLNKGFKRGRFKVEKQVYQKFEKSLIEKLNYLVFKDRK |
| | | ESNEPGHHLNAYQLTNKFLSFERLGKQSGVLFYATASYTSKVDPVTGFM |
| | | QNIYDPYHKEKTREFYKNFTKIVYNGNYFEFNYDLNSVKPDSEEKRYRT |
| | | NWTVCSCVIRSEYDSNSKTQKTYNVNDQLVKLFEDAKIKIENGNDLKST |
| | | ILEQDDKFIRDLHFYFIAIQKMRVVDSKIEKGEDSNDYIQSPVYPFYCS |
| | | KEIQPNKKGFYELPSNGDSNGAYNIARKGIVILDKIRLRVQIEKLFEDG |
| | | TKIDWQKLPNLISKVKDKKLLMTVFEEWAELTHQGEVQQGDLLGKKMSK |
| | | KGEQFAEFIKGLNVTKEDWEIYTQNEKVVQKQIKTWKLESNST |
| ART35 | 35 | MKAINEYYKQLGAYCREEGKEKDDFFKRIDGAYCAISHLFFGEHGEIAQ |
| | | SDSDVELIQKLLEAYKGLQRFIKPLLGHGDEADKDNEFDAKLRKVWDEL |
| | | DIITPLYDKVRNWLSRKIYNPEKIKLCFENNGKLLSGWVDSRTKSDNGT |
| | | QYGGYIFRKKNEIGEYDFYLGISADTKLFRRDAAISYDDGMYERLDYYQ |
| | | LKSKTLLGNSYVGDYGLDSMNLLSAFKNAAVKFQFEKEVVPKDKENVPK |
| | | YLKRLKLDYAGFYQILMNDDKVVDAYKIMKQHILATLTSSIRVPAAIEL |
| | | ATQKELGIDELIDEIMNLPSKSFGYFPIVTAAIEEANKRENKPLFLFKM |
| | | SNKDLSYAATASKGLRKGRGTENLHSMYLKALLGMTQSVFDIGSGMVFF |
| | | RHQTKGLAETTARHKANEFVANKNKLNDKKKSIFGYEIVKNKRFTVDKY |
| | | LFKLSMNLNYSQPNNNKIDVNSKVREIISNGGIKNIIGIDRGERNLLYL |
| | | SLIDLKGNIVMQKSLNILKDDHNAKETDYKGLLTEREGENKEARRNWKK |
| | | IANIKDLKRGYLSQVVHIISKMMVEYNAIVVLEDLNPGFIRGRQKIERN |
| | | VYEQFERMLIDKLNFYVDKHKGANETGGLLHALQLTSEFKNFKKSEHQN |
| | | GCLFYIPAWNTSKIDPATGFVNLENTKYTNAVEAQEFFSKEDEIRYNEE |
| | | KDWFEFEFDYDKFTQKAHGTRTKWTLCTYGMRLRSFKNSAKQYNWDSEV |
| | | VALTEEFKRILGEAGIDIHENLKDAICNLEGKSQKYLEPLMQFMKLLLQ |
| | | LRNSKAGTDEDYILSPVADENGIFYDSRSCGDQLPENADANGAYNIARK |
| | | GLMLIEQIKNAEDLNNVKFDISNKAWLNFAQQKPYKNGMKAINEYYKQL |
| | | GAYCREEGKEKDDFFKRIDGAYCAISHLFFGEHGEIAQSDSDVELIQKL |
| | | LEAYKGLQRFIKPLLGHGDEADKDNEFDAKLRKVWDELDIITPLYDKVR |
| | | NWLSRKIYNPEKIKLCFENNGKLLSGWVDSRTKSDNGTQYGGYIFRKKN |
| | | EIGEYDFYLGISADTKLFRRDAAISYDDGMYERLDYYQLKSKTLLGNSY |
| | | VGDYGLDSMNLLSAFKNAAVKFQFEKEVVPKDKENVPKYLKRLKLDYAG |
| | | FYQILMNDDKVVDAYKIMKQHILATLTSSIRVPAAIELATQKELGIDEL |
| | | IDEIMNLPSKSFGYFPIVTAAIEEANKRENKPLFLFKMSNKDLSYAATA |
| | | SKGLRKGRGTENLHSMYLKALLGMTQSVFDIGSGMVFFRHQTKGLAETT |
| | | ARHKANEFVANKNKLNDKKKSIFGYEIVKNKRFTVDKYLFKLSMNLNYS |
| | | QPNNNKIDVNSKVREIISNGGIKNIIGIDRGERNLLYLSLIDLKGNIVM |
| | | QKSLNILKDDHNAKETDYKGLLTEREGENKEARRNWKKIANIKDLKRGY |
| | | LSQVVHIISKMMVEYNAIVVLEDLNPGFIRGRQKIERNVYEQFERMLID |
| | | KLNFYVDKHKGANETGGLLHALQLTSEFKNFKKSEHQNGCLFYIPAWNT |
| | | SKIDPATGFVNLENTKYTNAVEAQEFFSKFDEIRYNEEKDWFEFEFDYD |
| | | KFTQKAHGTRTKWTLCTYGMRLRSFKNSAKQYNWDSEVVALTEEFKRIL |
| | | GEAGIDIHENLKDAICNLEGKSQKYLEPLMQFMKLLLQLRNSKAGTDED |
| | | YILSPVADENGIFYDSRSCGDQLPENADANGAYNIARKGLMLIEQIKNA |
| | | EDLNNVKFDISNKAWLNFAQQKPYKNG |
| ART11* | 36 | MYYQGLTKLYPISKTIRNELIPVGKTLEHIRMNNILEADIQRKSDYERV |
| | | KKLMDDYHKQLINESLQDVHLSYVEEAADLYLNASKDKDIVDKFSKCQD |
| | | KLRKEIVNLLKSHENFPKIGNKEIIKLLQSLSDTEKDYNALDSFSKFYT |
| | | YFTSYNEVRKNLYSDEEKSSTAAYRLINENLPKFLDNIKAYSIAKSAGV |
| | | RAKELTEEEQDCLEMTETFERTLTQDGIDNYNELIGKLNFAINLYNQQN |
| | | NKLKGFRKVPKMKELYKQILSEREASFVDEFVDDEALLTNVESFSAHIK |
| | | EFLESDSLSRFAEVLEESGGEMVYIKNDTSKTTFSNIVEGSWNVIDERL |
| | | AEEYDSANSKKKKDEKYYDKRHKELKKNKSYSVEKIVSLSTETEDVIGK |
| | | YIEKLQADIIAIKETREVFEKVVLKEHDKNKSLRKNTKAIEAIKSFLDT |
| | | IKDFERDIKLISGSEHEMEKNLAVYAEQENILSSIRNVDSLYNMSRNYL |
| | | TQKPFSTEKFKLNFNRATLLNGWDKNKETDNLGILLVKEGKYYLGIMNT |
| | | KANKSFVNPPKPKTDNVYHKVNYKLLPGPNKMLPKVFFAKSNLEYYKPS |
| | | EDLLAKYQAGTHKKGENFSLEDCHSLISFFKDSLEKHPDWSEFGFKFSD |
| | | TKKYDDLSGFYREVEKQGYKITYTDIDVEYIDSLVEKDELYFFQIYNKD |
| | | FSPYSKGNYNLHTLYLTMLFDERNLRNVVYKLNGEAEVFYRPASIGKDE |
| | | LIIHKSGEEIKNKNPKRAIDKPTSTFEYDIVKDRRYTKDKFMLHIPVTM |
| | | NFGVDETRRFNEVVNDAIRGDDKVRVIGIDRGERNLLYVVVVDSDGTIL |
| | | EQISLNSIINNEYSIETDYHKLLDEKEGDRDRARKNWTTIENIKELKEG |
| | | YLSQVVNVIAKLVLKYDAIICLEDLNFGFKRGRQKVEKQVYQKFEKMLI |
| | | DKLNYLVIDKSRSQENPEEVGHVLNALQLTSKFTSFKELGKQTGIIYYV |
| | | PAYLTSKIDPTTGFANLFYVKYESVEKSKDFFNREDSICENKVAGYFEF |
| | | SFDYKNFTDRACGMRSKWKVCTNGERIIKYRNEEKNSSFDDKVIVLTEE |
| | | FKKLFNEYGIAFNDCMDLTDAINAIDDASFFRKLTKLFQQTLQMRNSSA |
| | | DGSRDYIISPVENDNGEFFNSEKCDKSKPKDADANGAFNIARKGLWVLE |
| | | QLYNSSSGEKLNLAMTNAEWLEYAQQHTI |

In certain embodiments, a Cas nuclease comprises ABW1 (SEQ ID NO: 3), ABW2 (SEQ ID NO: 16), ABW3 (SEQ ID NO: 29), ABW4 (SEQ ID NO: 42), ABW5 (SEQ ID NO: 55), ABW6 (SEQ ID NO: 68), ABW7 (SEQ ID NO: 81), ABW8 (SEQ ID NO: 94), or ABW9 (SEQ ID NO: 107) (all SEQ ID NOs for ABW1-9 and variants thereof from International (PCT) Application Publication No. WO 2021/108324), or variants thereof, such as any one of variants 1-10 of ABW1 (SEQ ID NOs: 4-13, respectively), any one of variants 1-10 of ABW2 (SEQ ID NOs: 17-26, respectively), any one of variants 1-10 of ABW3 (SEQ ID NOs: 30-39, respectively), any one of variants 1-10 of ABW4 (SEQ ID NOs: 43-52, respectively), any one of variants 1-10 of ABW5 (SEQ ID NOs: 56-65, respectively), any one of variants 1-10 of ABW6 (SEQ ID NOs: 69-78, respectively), any one of variants 1-10 of ABW7 (SEQ ID NOs: 82-91, respectively), any one of variants 1-10 of ABW8 (SEQ ID NOs: 95-104, respectively), any one of variants 1-10 of ABW9 (SEQ ID NOs: 108-117, respectively). ABW1-ABW9, and variants thereof are known in the art and are described in International (PCT) Application Publication No. WO 2021/108324.

More type V-A Cas nucleases and their corresponding naturally occurring CRISPR-Cas systems can be identified by computational and experimental methods known in the art, e.g., as described in U.S. Pat. No. 9,790,490 and Shmakov et al. (2015) MOL. CELL, 60:385. Exemplary computational methods include analysis of putative Cas proteins by homology modeling, structural BLAST, PSI-BLAST, or HHPred, and analysis of putative CRISPR loci by identification of CRISPR arrays. Exemplary experimental methods include in vitro cleavage assays and in-cell nuclease assays (e.g., the Surveyor assay) as described in Zetsche et al. (2015) CELL, 163:759.

In certain embodiments, the Cas protein is a Cas nuclease that directs cleavage of one or both strands at the target locus, such as the target strand (i.e., the strand having the target nucleotide sequence that is at least partially complementary to and can hybridize with a single guide nucleic acid or dual guide nucleic acids) and/or the non-target strand. In certain embodiments, the Cas nuclease directs cleavage of one or both strands within about 1, 2, 3, 4, 5, 6, 7, 8, 9, 10, 15, 20, 25, 50, 100, 200, 500, or more nucleotides from the first or last nucleotide of the target nucleotide sequence or its complementary sequence. In certain embodiments, the cleavage is staggered, i.e. generating sticky ends. In certain embodiments, the cleavage generates a staggered cut with a 5′ overhang. In certain embodiments, the cleavage generates a staggered cut with a 5′ overhang of 1 to 5 nucleotides, e.g., of 4 or 5 nucleotides. In certain embodiments, the cleavage site is distant from the PAM, e.g., the cleavage occurs after the 18th nucleotide on the non-target strand and after the 23rd nucleotide on the target strand.

In certain embodiments, a composition provided herein comprises a Cas nuclease that a compatible guide nucleic acid (gNA), e.g., a gRNA, is capable of activating. In certain embodiments, a composition provided herein further comprises a Cas protein that is related to the Cas nuclease that a compatible guide nucleic acid (gNA), e.g., a gRNA, is capable of activating. For example, in certain embodiments, a Cas protein comprises an amino acid sequence at least 80% (e.g., at least 85%, at least 90%, at least 91%, at least 92%, at least 93%, at least 94%, at least 95%, at least 96%, at least 97%, at least 98%, or at least 99%) identical to the Cas nuclease amino acid sequence. In certain embodiments, a Cas protein comprises a nuclease-inactive mutant of the Cas nuclease. In certain embodiments, a Cas protein further comprises an effector domain.

In certain embodiments, a Cas protein lacks substantially all DNA cleavage activity. Such a Cas protein can be generated, e.g., by introducing one or more mutations to an active Cas nuclease (e.g., a naturally occurring Cas nuclease). A mutated Cas protein is considered to lack substantially all DNA cleavage activity when the DNA cleavage activity of the protein has no more than about 25%, 10%, 5%, 1%, 0.1%, 0.01%, or less of the DNA cleavage activity of the corresponding non-mutated form, for example, nil or negligible as compared with the non-mutated form. Thus, a Cas protein may comprise one or more mutations (e.g., a mutation in the RuvC domain of a type V-A Cas protein) and be used as a generic DNA binding protein with or without fusion to an effector domain. Exemplary mutations include D908A, E993A, and D1263A with reference to the amino acid positions in AsCpf1; D832A, E925A, and D1180A with reference to the amino acid positions in LbCpf1; and D917A, E1006A, and D1255A with reference to the amino acid position numbering of the FnCpf1. More mutations can be designed and generated according to the crystal structure described in Yamano et al. (2016) CELL, 165:949.

It is understood that a Cas protein, rather than losing nuclease activity to cleave all DNA, may lose the ability to cleave only the target strand or only the non-target strand of a double-stranded DNA, thereby being functional as a nickase (see, Gao et al. (2016) CELL RES., 26:901). Accordingly, in certain embodiments, a Cas nuclease is a Cas nickase. In certain embodiments, a Cas nuclease has the activity to cleave the non-target strand but lacks substantially the activity to cleave the target strand, e.g., by a mutation in the Nuc domain. In certain embodiments, a Cas nuclease has the cleavage activity to cleave the target strand but lacks substantially the activity to cleave the non-target strand.

In certain embodiments, a Cas nuclease has the activity to cleave a double-stranded DNA and result in a double-strand break.

Cas proteins that lack substantially all DNA cleavage activity or have the ability to cleave only one strand may also be identified from naturally occurring systems. For example, certain naturally occurring CRISPR-Cas systems may retain the ability to bind the target nucleotide sequence but lose entire or partial DNA cleavage activity in eukaryotic (e.g., mammalian or human) cells. Such type V-A proteins are disclosed, for example, in Kim et al. (2017) ACS SYNTH. BIOL. 6 (7): 1273-82 and Zhang et al. (2017) CELL DISCOV. 3:17018.

The activity of a Cas protein (e.g., Cas nuclease) can be altered, e.g., by creating an engineered Cas protein. In certain embodiments, altered activity of an engineered Cas protein comprises increased targeting efficiency and/or decreased off-target binding. While not wishing to be bound by theory, it is hypothesized that off-target binding can be recognized by the Cas protein, for example, by the presence of one or more mismatches between the spacer sequence and the target nucleotide sequence, which may affect the stability and/or conformation of the CRISPR-Cas complex. In certain embodiments, altered activity comprises modified binding, e.g., increased binding to the target locus (e.g., the target strand or the non-target strand) and/or decreased binding to off-target loci. In certain embodiments, altered activity comprises altered charge in a region of the protein that associates with a single guide nucleic acid or dual guide nucleic acids. In certain embodiments, altered activity of an engineered Cas protein comprises altered charge in a region of the protein that associates with the target strand and/or the non-target strand. In certain embodiments, altered activity of an engineered Cas protein comprises altered charge in a region of the protein that associates with an off-target locus. The altered charge can include decreased positive charge, decreased negative charge, increased positive charge, or increased negative charge. For example, decreased negative charge and increased positive charge may generally strengthen binding to the nucleic acid(s) whereas decreased positive charge and increased negative charge may weaken binding to the nucleic acid(s). In certain embodiments, altered activity comprises increased or decreased steric hindrance between the protein and a single guide nucleic acid or dual guide nucleic acids. In certain embodiments, altered activity comprises increased or decreased steric hindrance between the protein and the target strand and/or the non-target strand. In certain embodiments, altered activity comprises increased or decreased steric hindrance between the protein and an off-target locus. In certain embodiments, a modification or mutation comprises one or more substitutions of Lys, His, Arg, Glu, Asp, Ser, Gly, and/or Thr. In certain embodiments, a modification or mutation comprises one or more substitutions with Gly, Ala, Ile, Glu, and/or Asp. In certain embodiments, modification or mutation comprises one or more amino acid substitutions in the groove between the WED and RuvC domain of the Cas protein (e.g., a type V-A Cas protein).

In certain embodiments, altered activity of an engineered Cas protein comprises increased nuclease activity to cleave the target locus. In certain embodiments, altered activity of an engineered Cas protein comprises decreased nuclease activity to cleave an off-target locus. In certain embodiments, altered activity of an engineered Cas protein comprises altered helicase kinetics. In certain embodiments, an engineered Cas protein comprises a modification that alters formation of the CRISPR complex.

In certain embodiments, a protospacer adjacent motif (PAM) or PAM-like motif directs binding of a Cas protein complex to a target locus. Many Cas proteins have PAM specificity. The precise sequence and length requirements for the PAM differ depending on the Cas protein used. PAM sequences are typically 2-5 base pairs in length and are adjacent to (but located on a different strand of target DNA from) the target nucleotide sequence. PAM sequences can be identified using any suitable method, such as testing cleavage, targeting, or modification of oligonucleotides having the target nucleotide sequence and different PAM sequences.

Exemplary PAM sequences are provided in Tables 2 and 3. In certain embodiments, a Cas protein comprises MAD7 and the PAM is TTTN, wherein N is A, C, G, or T. In certain embodiments, a Cas protein comprises MAD7 and the PAM is CTTN, wherein N is A, C, G, or T. In certain embodiments, a Cas protein comprises AsCpf1 and the PAM is TTTN, wherein Nis A, C, G, or T. In certain embodiments, a Cas protein comprises FnCpf1 and the PAM is 5′ TTN, wherein N is A, C, G, or T. PAM sequences for certain other type V-A Cas proteins are disclosed in Zetsche et al. (2015) CELL, 163:759 and U.S. Pat. No. 9,982,279. Further, engineering of the PAM Interacting (PI) domain of a Cas protein may allow programing of PAM specificity, improve target site recognition fidelity, and/or increase the versatility of an engineered, non-naturally occurring system. Exemplary approaches to alter the PAM specificity of Cpf1 are described in Gao et al. (2017) NAT. BIOTECHNOL., 35:789.

In certain embodiments, an engineered Cas protein comprises a modification that alters the Cas protein specificity in concert with modification to targeting range. Cas mutants can be designed to have increased target specificity as well as accommodating modifications in PAM recognition, for example by choosing mutations that alter PAM specificity (e.g., in the PI domain) and combining those mutations with groove mutations that increase (or if desired, decrease) specificity for the on-target locus versus off-target loci. The Cas modifications described herein can be used to counter loss of specificity resulting from alteration of PAM recognition, enhance gain of specificity resulting from alteration of PAM recognition, counter gain of specificity resulting from alteration of PAM recognition, or enhance loss of specificity resulting from alteration of PAM recognition.

In certain embodiments, an engineered Cas protein comprises one or more nuclear localization signal (NLS) motifs. In certain embodiments, an engineered Cas protein comprises at least 2 (e.g., at least 3, at least 4, at least 5, at least 6, at least 7, at least 8, at least 9, or at least 10) NLS motifs. Non-limiting examples of NLS motifs include: the NLS of SV40 large T-antigen, having the amino acid sequence of PKKKRKV (SEQ ID NO: 40); the NLS from nucleoplasmin, e.g., the nucleoplasmin bipartite NLS having the amino acid sequence of KRPAATKKAGQAKKKK (SEQ ID NO: 41); the c-myc NLS, having the amino acid sequence of PAAKRVKLD (SEQ ID NO: 42) or RQRRNELKRSP (SEQ ID NO: 43); the hRNPA1 M9 NLS, having the amino acid sequence of NQSSNFGPMKGGNFGGRSSGPYGGGGQYFAKPRNQGGY (SEQ ID NO: 44); the importin-α IBB domain NLS, having the amino acid sequence of RMRIZFKNKGKDTAELRRRRVEVSVELRKAKKDEQILKRRNV (SEQ ID NO: 45); the myoma T protein NLS, having the amino acid sequence of VSRKRPRP (SEQ ID NO: 46) or PPKKARED (SEQ ID NO: 47); the human p53 NLS, having the amino acid sequence of PQPKKKPL (SEQ ID NO: 48); the mouse c-abl IV NLS, having the amino acid sequence of SALIKKKKKMAP (SEQ ID NO: 49); the influenza virus NS1 NLS, having the amino acid sequence of DRLRR (SEQ ID NO: 50) or PKQKKRK (SEQ ID NO: 51); the hepatitis virus 8 antigen NLS, having the amino acid sequence of RKLKKKIKKL (SEQ ID NO: 52); the mouse Mx1 protein NLS, having the amino acid sequence of REKKKFLKRR (SEQ ID NO: 53); the human poly(ADP-ribose) polymerase NLS, having the amino acid sequence of KRKGDEVDGVDEVAKKKSKK (SEQ ID NO: 54); the human glucocorticoid receptor NLS, having the amino acid sequence of RKCLQAGMNLEARKTKK (SEQ ID NO: 55), and synthetic NLS motifs such as PAAKKKKLD (SEQ ID NO: 56).

In general, the one or more NLS motifs are of sufficient strength to drive accumulation of the Cas protein in a detectable amount in the nucleus of a eukaryotic cell. The strength of nuclear localization activity may derive from the number of NLS motif(s) in the Cas protein, the particular NLS motif(s) used, the position(s) of the NLS motif(s), or a combination of these and/or other factors. In certain embodiments, an engineered Cas protein comprises at least 1 (e.g., at least 2, at least 3, at least 4, at least 5, at least 6, at least 7, at least 8, at least 9, or at least 10) NLS motif(s) at or near the N-terminus (e.g., within about 1, 2, 3, 4, 5, 10, 15, 20, 25, 30, 40, 50, or more amino acids along the polypeptide chain from the N-terminus). In certain embodiments, an engineered Cas protein comprises at least 1 (e.g., at least 2, at least 3, at least 4, at least 5, at least 6, at least 7, at least 8, at least 9, or at least 10) NLS motif(s) at or near the C-terminus (e.g., within about 1, 2, 3, 4, 5, 10, 15, 20, 25, 30, 40, 50, or more amino acids along the polypeptide chain from the C-terminus). In certain embodiments, an engineered Cas protein comprises at least 1 (e.g., at least 2, at least 3, at least 4, at least 5, at least 6, at least 7, at least 8, at least 9, or at least 10) NLS motif(s) at or near the C-terminus and at least 1 (e.g., at least 2, at least 3, at least 4, at least 5, at least 6, at least 7, at least 8, at least 9, or at least 10) NLS motif(s) at or near the N-terminus. In certain embodiments, the engineered Cas protein comprises one, two, or three NLS motifs at or near the C-terminus. In certain embodiments, the engineered Cas protein comprises one NLS motif at or near the N-terminus and one, two, or three NLS motifs at or near the C-terminus. In certain embodiments, the engineered Cas protein comprises a nucleoplasmin NLS at or near the C-terminus.

Detection of accumulation in the nucleus may be performed by any suitable technique. For example, a detectable marker may be fused to a nucleic acid-targeting protein, such that location within a cell may be visualized. Cell nuclei may also be isolated from cells, the contents of which may then be analyzed by any suitable process for detecting the protein, such as immunohistochemistry, Western blot, or enzyme activity assay. Accumulation in the nucleus may also be determined indirectly, such as by an assay that detects the effect of the nuclear import of a Cas protein complex (e.g., assay for DNA cleavage or mutation at the target locus, or assay for altered gene expression activity) as compared to a control not exposed to the Cas protein or exposed to a Cas protein lacking one or more of the NLS motifs.

A Cas protein may comprise a chimeric Cas protein, e.g., a Cas protein having enhanced function by being a chimera. Chimeric Cas proteins may be new Cas proteins containing fragments from more than one naturally occurring Cas protein or variants thereof. For example, fragments of multiple type V-A Cas homologs (e.g., orthologs) may be fused to form a chimeric Cas protein. In certain embodiments, a chimeric Cas protein comprises fragments of Cpf1 orthologs from multiple species and/or strains.

In certain embodiments, a Cas protein comprises one or more effector domains. The one or more effector domains may be located at or near the N-terminus of the Cas protein and/or at or near the C-terminus of the Cas protein. In certain embodiments, an effector domain comprised in the Cas protein is a transcriptional activation domain (e.g., VP64), a transcriptional repression domain (e.g., a KRAB domain or an SID domain), an exogenous nuclease domain (e.g., FokI), a deaminase domain (e.g., cytidine deaminase or adenine deaminase), or a reverse transcriptase domain (e.g., a high fidelity reverse transcriptase domain). Other activities of effector domains include but are not limited to methylase activity, demethylase activity, transcription release factor activity, translational initiation activity, translational activation activity, translational repression activity, histone modification (e.g., acetylation or demethylation) activity, single-stranded RNA cleavage activity, double-strand RNA cleavage activity, single-strand DNA cleavage activity, double-strand DNA cleavage activity, and nucleic acid binding activity.

In certain embodiments, a Cas protein comprises one or more protein domains that enhance homology-directed repair (HDR) and/or inhibit non-homologous end joining (NHEJ). Exemplary protein domains having such functions are described in Jayavaradhan et al. (2019) NAT. COMMUN. 10 (1): 2866 and Janssen et al. (2019) MOL. THER. NUCLEIC ACIDS 16:141-54. In certain embodiments, a Cas protein comprises a dominant negative version of p53-binding protein 1 (53BP1), for example, a fragment of 53BP1 comprising a minimum focus forming region (e.g., amino acids 1231-1644 of human 53BP1). In certain embodiments, a Cas protein comprises a motif that is targeted by APC-Cdh1, such as amino acids 1-110 of human Geminin, thereby resulting in degradation of the fusion protein during the HDR non-permissive G1 phase of the cell cycle.

In certain embodiments, a Cas protein comprises an inducible or controllable domain. Non-limiting examples of inducers or controllers include light, hormones, and small molecule drugs. In certain embodiments, a Cas protein comprises a light inducible or controllable domain. In certain embodiments, a Cas protein comprises a chemically inducible or controllable domain.

In certain embodiments, a Cas protein comprises a tag protein or peptide for ease of tracking and/or purification. Non-limiting examples of tag proteins and peptides include fluorescent proteins (e.g., green fluorescent protein (GFP), YFP, RFP, CFP, mCherry, tdTomato), HIS tags (e.g., 6×His tag, or gly-6×His; 8×His, or gly-8×His), hemagglutinin (HA) tag, FLAG tag, 3×FLAG tag, and Myc tag.

In certain embodiments, a Cas protein is conjugated to a non-protein moiety, such as a fluorophore useful for genomic imaging. In certain embodiments, a Cas protein is covalently conjugated to the non-protein moiety. The terms “CRISPR-Associated protein,” “Cas protein,” “Cas,” “CRISPR-Associated nuclease,” and “Cas nuclease” are used herein to include such conjugates despite the presence of one or more non-protein moieties.

### B. Guide Nucleic Acids

A guide nucleic acid can be a single gNA (sgNA, e.g., sgRNA), in which the gNA is a single polynucleotide, or a dual gNA (e.g., dual gRNA), in which the gNA comprises two separate polynucleotides (these can in some cases be covalently linked, but not via a conventional internucleotide linkage). In certain embodiments, a single guide nucleic acid is capable of activating a Cas nuclease alone (e.g., in the absence of a tracrRNA).

In general, a gNA comprises a modulator nucleic acid and a targeter nucleic acid. In a sgNA the modulator and targeter nucleic acids are part of a single polynucleotide. In a dual gNA the modulator and targeter nucleic acids are separate, e.g., not joined by a conventional nucleotide linkage, such as not joined at all. The targeter nucleic acid comprises a spacer sequence and a targeter stem sequence. The modulator nucleic acid comprises a modulator stem sequence and, generally, further nucleotides, such as nucleotides comprising a 5′ tail. The modulator stem sequence and targeter stem sequence can each comprise any suitable number of nucleotides and are of sufficient complementarity that they can hybridize. In a single gNA there may be additional NTs between the targeter stem sequence and the modulator stem sequence; these can, in certain cases, form secondary structure, such as a loop.

In certain embodiments, the guide nucleic acid comprises a targeter nucleic acid that, in combination with a modulator nucleic acid, is capable of binding a Cas protein. In certain embodiments, the guide nucleic acid comprises a targeter nucleic acid that, in combination with a modulator nucleic acid, is capable of activating a Cas nuclease. In certain embodiments, the system further comprises the Cas protein that the targeter nucleic acid and the modulator nucleic acid are capable of binding or the Cas nuclease that the targeter nucleic acid and the modulator nucleic acid are capable of activating.

It is contemplated that the single or dual guide nucleic acids need to be the compatible with a Cas protein (e.g., Cas nuclease) to provide an operative CRISPR system. For example, the targeter stem sequence and the modulator stem sequence can be derived from a naturally occurring crRNA capable of activating a Cas nuclease in the absence of a tracrRNA. Alternatively, the targeter stem sequence and the modulator stem sequence can be derived from a naturally occurring set of crRNA and tracrRNA, respectively, that are capable of activating a Cas nuclease. In certain embodiments, the nucleotide sequences of the targeter stem sequence and the modulator stem sequence are identical to the corresponding stem sequences of a stem-loop structure in such naturally occurring crRNA.

Guide nucleic acid sequences that are operative with a type II or type V Cas protein are known in the art and are disclosed, for example, in U.S. Pat. Nos. 9,790,490, 9,896,696, 10,113,179, and 10,266,850, and U.S. Patent Application Publication No. 2014/0242664. It is understood that these sequences are merely illustrative, and other guide nucleic acid sequences may also be used with these Cas proteins.

TABLE 2
Type V-A Cas Protein and Corresponding Single Guide Nucleic Acid Sequences

| Cas Protein | Scaffold Sequence¹ | PAM² |
|---|---|---|
| MAD7 (SEQ ID | UAAUUUCUACUCUUGUAGA (SEQ ID NO: 57), | 5′ TTTN |
| NO: 37) | AUCUACAACAGUAGA (SEQ ID NO: 58), | or 5′ |
| | AUCUACAAAAGUAGA (SEQ ID NO: 59), | CTTN |
| | GGAAUUUCUACUCUUGUAGA (SEQ ID NO: 60), | |
| | UAAUUCCCACUCUUGUGGG (SEQ ID NO: 61) | |
| MAD2 (SEQ ID | AUCUACAAGAGUAGA (SEQ ID NO: 62), | 5′ TTTN |
| NO: 38) | AUCUACAACAGUAGA (SEQ ID NO: 58), | |
| | AUCUACAAAAGUAGA (SEQ ID NO: 59), | |
| | AUCUACACUAGUAGA (SEQ ID NO: 63) | |
| AsCpf1 (SEQ | UAAUUUCUACUCUUGUAGA (SEQ ID NO: 57) | 5′ TTTN |
| ID NO: 3 of | | |
| WO | | |
| 2021/158918) | | |
| LbCpf1 (SEQ | UAAUUUCUACUAAGUGUAGA (SEQ ID NO: 64) | 5′ TTTN |
| ID NO: 4 of | | |
| WO | | |
| 2021/158918) | | |
| FnCpf1 (SEQ | UAAUUUUCUACUUGUUGUAGA (SEQ ID NO: 65) | 5′ TTN |
| ID NO: 5 of | | |
| WO | | |
| 2021/158918) | | |
| PbCpf1 (SEQ | AAUUUCUACUGUUGUAGA (SEQ ID NO: 66) | 5′ TTTC |
| ID NO: 6 of | | |
| WO | | |
| 2021/158918) | | |
| PsCpf1 (SEQ | AAUUUCUACUGUUGUAGA (SEQ ID NO: 66) | 5′ TTTC |
| ID NO: 7 of | | |
| WO | | |
| 2021/158918) | | |
| As2Cpf1 (SEQ | AAUUUCUACUGUUGUAGA (SEQ ID NO: 66) | 5′ TTTC |
| ID NO: 8 of | | |
| WO | | |
| 2021/158918) | | |
| McCpf1 (SEQ | GAAUUUCUACUGUUGUAGA (SEQ ID NO: 67) | 5′ TTTC |
| ID NO: 9 of | | |
| WO | | |
| 2021/158918) | | |
| Lb3Cpf1 (SEQ | GAAUUUCUACUGUUGUAGA (SEQ ID NO: 67) | 5′ TTTC |
| ID NO: 10 of | | |
| WO | | |
| 2021/158918) | | |
| EcCpf1 (SEQ | GAAUUUCUACUGUUGUAGA (SEQ ID NO: 67) | 5′ TTTC |
| ID NO: 11 of | | |
| WO | | |
| 2021/158918) | | |
| SmCsm1 (SEQ | GAAUUUCUACUGUUGUAGA (SEQ ID NO: 67) | 5′ TTTC |
| ID NO: 12 of | | |
| WO | | |
| 2021/158918) | | |
| SsCsm1 (SEQ | GAAUUUCUACUGUUGUAGA (SEQ ID NO: 67) | 5′ TTTC |
| ID NO: 13 of | | |
| WO | | |
| 2021/158918) | | |
| MbCsm1 (SEQ | GAAUUUCUACUGUUGUAGA (SEQ ID NO: 67) | 5′ TTTC |
| ID NO: 14 of | | |
| WO | | |
| 2021/158918) | | |
| ART2 (SEQ ID | GUCUAAAGGUACCACCAAAUUUCUACUGUUGUAGAU | 5′ TTTN |
| NO: 2 | (SEQ ID NO: 68) | or 5′ |
| | | NTTN |
| ART11 (SEQ ID | GCUUAGAACCUUUAAAUAAUUUCUACUAUUGUAGAU | 5′ TTTN |
| NO: 11 | (SEQ ID NO: 69) | or 5′ |
| | | NTTN |
| ART11* (SEQ | GCUUAGAACCUUUAAAUAAUUUCUACUAUUGUAGAU | 5′ TTTN |
| | | or 5′ |
| ID NO: 36 | (SEQ ID NO: 69) | NTTN |

¹The modulator sequence in the scaffold sequence is underlined; the targeter stem sequence in the scaffold sequence is bold-underlined. It is understood that a “scaffold sequence” listed herein constitutes a portion of a single guide nucleic acid. Additional nucleotide sequences, other than the spacer sequence, can be comprised in the single guide nucleic acid.
²In the consensus PAM sequences, N represents A, C, G, or T. Where the PAM sequence is preceded by “5′,” it means that the PAM is located immediately upstream of the target nucleotide sequence when using the non-target strand (i.e., the strand not hybridized with the spacer sequence) as the coordinate.

TABLE 3
Type V-A Cas Protein and Corresponding Dual Guide Nucleic Acid Sequences

| | | Targeter | |
|---|---|---|---|
| | | Stem | |
| Cas Protein | Modulator Sequence¹ | Sequence | PAM² |
| MAD7 (SEQ ID NO: | UAAUUUCUAC (SEQ ID NO: | GUAGA | 5′ TTTN |
| 37) | 70) | | or 5′ |
| | AUCUAC (SEQ ID NO: 71) | GUAGA | CTTN |
| | GGAAUUUCUAC (SEQ ID NO: | GUAGA | |
| | 72) | | |
| | UAAUUCCCAC (SEQ ID NO: | GUGGG | |
| | 73) | | |
| MAD2 (SEQ ID NO: | AUCUAC (SEQ ID NO: 71) | GUAGA | 5′ TTTN |
| 38) | | | |
| AsCpf1 (SEQ ID NO: | UAAUUUCUAC (SEQ ID NO: | GUAGA | 5′ TTTN |
| 3 of WO | 70) | | |
| 2021/158918) | | | |
| LbCpf1 (SEQ ID NO: | UAAUUUCUAC (SEQ ID NO: | GUAGA | 5′ TTTN |
| 4 of WO | 70) | | |
| 2021/158918) | | | |
| FnCpf1 (SEQ ID NO: | UAAUUUUCUACU (SEQ ID NO: | GUAGA | 5′ TTN |
| 5 of WO | 74) | | |
| 2021/158918) | | | |
| PbCpf1 (SEQ ID NO: | AAUUUCUAC (SEQ ID NO: 75) | GUAGA | 5′ TTTC |
| 6 of WO | | | |
| 2021/158918) | | | |
| PsCpf1 (SEQ ID NO: | AAUUUCUAC (SEQ ID NO: 75) | GUAGA | 5′ TTTC |
| 7 of WO | | | |
| 2021/158918) | | | |
| As2Cpf1 (SEQ ID | AAUUUCUAC (SEQ ID NO: 75) | GUAGA | 5′ TTTC |
| NO: 8 of WO | | | |
| 2021/158918) | | | |
| McCpf1 (SEQ ID NO: | GAAUUUCUAC (SEQ ID NO: | GUAGA | 5′ TTTC |
| 9 of WO | 76 | | |
| 2021/158918) | | | |
| Lb3Cpf1 (SEQ ID | GAAUUUCUAC (SEQ ID NO: | GUAGA | |
| NO: 10 of WO | | | |
| 2021/158918) | 76) | | 5′ TTTC |
| EcCpf1 (SEQ ID NO: | GAAUUUCUAC (SEQ ID NO: | GUAGA | |
| 11 of WO | | | |
| 2021/158918) | 76) | | 5′ TTTC |
| SmCsm1 (SEQ ID NO: | GAAUUUCUAC (SEQ ID NO: | GUAGA | |
| 12 of WO | | | |
| 2021/158918) | 76) | | 5′ TTTC |
| SsCsm1 (SEQ ID NO: | GAAUUUCUAC (SEQ ID NO: | GUAGA | |
| 13 of WO | | | |
| 2021/158918) | 76) | | 5′ TTTC |
| MbCsm1 (SEQ ID NO: | GAAUUUCUAC (SEQ ID NO: | GUAGA | |
| 14 of WO | | | |
| 2021/158918) | 76) | | 5′ TTTC |
| ART2 (SEQ ID NO: 2) | AAAUUUCUAC (SEQ ID NO: | GUAGA | 5′ TTTN |
| | 77) | | or 5′ |
| | | | NTTN |
| ART11 (SEQ ID NO: | UAAUUUCUAC (SEQ ID NO: | | 5′ TTTN |
| 11) | 70) | | or 5′ |
| | | GUAGA | NTTN |
| ART11* (SEQ ID NO: | UAAUUUCUAC (SEQ ID NO: | | 5′ TTTN |
| 36) | 70) | | or 5′ |
| | | GUAGA | NTTN |

¹It is understood that a “modulator sequence” listed herein may constitute the nucleotide sequence of a modulator nucleic acid. Alternatively, additional nucleotide sequences can be comprised in the modulator nucleic acid 5′ and/or 3′ to a “modulator sequence” listed herein.
²In the consensus PAM sequences, N represents A, C, G, or T. Where the PAM sequence is preceded by “5′,” it means that the PAM is located immediately upstream of the target nucleotide sequence when using the non-target strand (i.e., the strand not hybridized with the spacer sequence) as the coordinate.

In certain embodiments, a guide nucleic acid, in the context of a type V-A CRISPR-Cas system, comprises a targeter stem sequence listed in Table 3. The same targeter stem sequences, as a portion of scaffold sequences, are bold-underlined in Table 2.

In certain embodiments, a guide nucleic acid is a single guide nucleic acid that comprises, from 5′ to 3′, a modulator stem sequence, a loop sequence, a targeter stem sequence, and a spacer sequence. In certain embodiments, the targeter stem sequence in the single guide nucleic acid is listed in Table 2 as a bold-underlined portion of scaffold sequence, and the modulator stem sequence is complementary (e.g., 100% complementary) to the targeter stem sequence. In certain embodiments, the single guide nucleic acid comprises, from 5′ to 3′, a modulator sequence listed in Table 2 as an underlined portion of a scaffold sequence, a loop sequence, a targeter stem sequence a bold-underlined portion of the same scaffold sequence, and a spacer sequence. In certain embodiments, an engineered, non-naturally occurring system comprises a single guide nucleic acid comprising a scaffold sequence listed in Table 2. In certain embodiments, the system further comprises a Cas protein (e.g., Cas nuclease) comprising an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in the SEQ ID NO listed in the same line of Table 2. In certain embodiments, the system further comprises a Cas protein (e.g., Cas nuclease) comprising the amino acid sequence set forth in the SEQ ID NO listed in the same line of Table 2. In certain embodiments, the system is useful for targeting, editing, or modifying a nucleic acid comprising a target nucleotide sequence close or adjacent to (e.g., immediately downstream of) a PAM listed in the same line of Table 2 when using the non-target strand (i.e., the strand not hybridized with the spacer sequence) as the coordinate.

In certain embodiments, a guide nucleic acid, e.g, dual gNA, comprises a targeter guide nucleic acid that comprises, from 5′ to 3′, a targeter stem sequence and a spacer sequence. In certain embodiments, the targeter stem sequence in the targeter nucleic acid is listed in Table 3. In certain embodiments, an engineered, non-naturally occurring system comprises the targeter nucleic acid and a modulator stem sequence complementary (e.g., 100% complementary) to the targeter stem sequence. In certain embodiments, the modulator nucleic acid comprises a modulator sequence listed in the same line of Table 3. In certain embodiments, the system further comprises a Cas protein (e.g., Cas nuclease) comprising an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in the SEQ ID NO listed in the same line of Table 3. In certain embodiments, the system further comprises a Cas protein (e.g., Cas nuclease) comprising the amino acid sequence set forth in the SEQ ID NO listed in the same line of Table 3. In certain embodiments, the system is useful for targeting, editing, or modifying a nucleic acid comprising a target nucleotide sequence close or adjacent to (e.g., immediately downstream of) a PAM listed in the same line of Table 3 when using the non-target strand (i.e., the strand not hybridized with the spacer sequence) as the coordinate.

A single guide nucleic acid, the targeter nucleic acid, and/or the modulator nucleic acid can be synthesized chemically or produced in a biological process (e.g., catalyzed by an RNA polymerase in an in vitro reaction). Such reaction or process may limit the lengths of the single guide nucleic acid, targeter nucleic acid, and/or modulator nucleic acid. In certain embodiments, a single guide nucleic acid is no more than 100, 90, 80, 70, 60, 50, 40, 30, or 25 nucleotides in length. In certain embodiments, a single guide nucleic acid is at least 20, 25, 30, 40, 50, 60, 70, 80, or 90 nucleotides in length. In certain embodiments, the single guide nucleic acid is 20-100, 20-90, 20-80, 20-70, 20-60, 20-50, 20-40, 20-30, 20-25, 25-100, 25-90, 25-80, 25-70, 25-60, 25-50, 25-40, 25-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 nucleotides in length. In certain embodiments, a targeter nucleic acid is no more than 100, 90, 80, 70, 60, 50, 40, 30, or 25 nucleotides in length. In certain embodiments, a targeter nucleic acid is at least 20, 25, 30, 40, 50, 60, 70, 80, or 90 nucleotides in length. In certain embodiments, the targeter nucleic acid is 20-100, 20-90, 20-80, 20-70, 20-60, 20-50, 20-40, 20-30, 20-25, 25-100, 25-90, 25-80, 25-70, 25-60, 25-50, 25-40, 25-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 nucleotides in length. In certain embodiments, a modulator nucleic acid is no more than 100, 90, 80, 70, 60, 50, 40, 30, or 20 nucleotides in length. In certain embodiments, a modulator nucleic acid is at least 10, 15, 20, 25, 30, 40, 50, 60, 70, 80, or 90 nucleotides in length. In certain embodiments, the modulator nucleic acid is 10-100, 10-90, 10-80, 10-70, 10-60, 10-50, 10-40, 10-30, 10-20, 15-100, 15-90, 15-80, 15-70, 15-60, 15-50, 15-40, 15-30, 15-20, 20-100, 20-90, 20-80, 20-70, 20-60, 20-50, 20-40, 20-30, 25-100, 25-90, 25-80, 25-70, 25-60, 25-50, 25-40, 25-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 nucleotides in length.

It is contemplated that the length of the duplex formed within the single guide nuclei acid or formed between the targeter nucleic acid and the modulator nucleic acid, e.g. in a dual gNA, may be a factor in providing an operative CRISPR system. In certain embodiments, the targeter stem sequence and the modulator stem sequence each consist of 4-10 nucleotides that base pair with each other. In certain embodiments, the targeter stem sequence and the modulator stem sequence each consist of 4-9, 4-8, 4-7, 4-6, 4-5, 5-10, 5-9, 5-8, 5-7, or 5-6 nucleotides that base pair with each other. In certain embodiments, the targeter stem sequence and the modulator stem sequence each consist of 4, 5, 6, 7, 8, 9, or 10 nucleotides. It is understood that the composition of the nucleotides in each sequence affects the stability of the duplex, and a C-G base pair confers greater stability than an A-U base pair. In certain embodiments, 20%-80%, 20%-70%, 20%-60%, 20%-50%, 20%-40%, 20%-30%, 30%-80%, 30%-70%, 30%-60%, 30%-50%, 30%-40%, 40%-80%, 40%-70%, 40%-60%, 40%-50%, 50%-80%, 50%-70%, 50%-60%, 60%-80%, 60%-70%, or 70%-80% of the base pairs are C-G base pairs.

In certain embodiments, the targeter stem sequence and the modulator stem sequence each consist of 5 nucleotides. As such, the targeter stem sequence and the modulator stem sequence form a duplex of 5 base pairs. In certain embodiments, 0-4, 0-3, 0-2, 0-1, 1-5, 1-4, 1-3, 1-2, 2-5, 2-4, 2-3, 3-5, 3-4, or 4-5 out of the 5 base pairs are C-G base pairs. In certain embodiments, 0, 1, 2, 3, 4, or 5 out of the 5 base pairs are C-G base pairs. In certain embodiments, the targeter stem sequence consists of 5′-GUAGA-3′ and the modulator stem sequence consists of 5′-UCUAC-3′. In certain embodiments, the targeter stem sequence consists of 5′-GUGGG-3′ and the modulator stem sequence consists of 5′-CCCAC-3′.

In certain embodiments, in a type V-A system, the 3′ end of the targeter stem sequence is linked by no more than 1, 2, 3, 4, 5, 6, 7, 8, 9, or 10 nucleotides to the 5′ end of the spacer sequence. In certain embodiments, the targeter stem sequence and the spacer sequence are adjacent to each other, directly linked by an internucleotide bond. In certain embodiments, the targeter stem sequence and the spacer sequence are linked by one nucleotide, e.g., a uridine. In certain embodiments, the targeter stem sequence and the spacer sequence are linked by two or more nucleotides. In certain embodiments, the targeter stem sequence and the spacer sequence are linked by 1, 2, 3, 4, 5, 6, 7, 8, 9, or 10 nucleotides.

In certain embodiments, the targeter nucleic acid further comprises an additional nucleotide sequence 5′ to the targeter stem sequence. In certain embodiments, the additional nucleotide sequence comprises at least 1 (e.g., at least 2, at least 3, at least 4, at least 5, at least 6, at least 7, at least 8, at least 9, at least 10, at least 15, at least 20, at least 25, at least 30, at least 35, at least 40, at least 45, or at least 50) nucleotides. In certain embodiments, the additional nucleotide sequence consists of 1, 2, 3, 4, 5, 6, 7, 8, 9, 10, 15, 20, 25, 30, 35, 40, 45, or 50 nucleotides. In certain embodiments, the additional nucleotide sequence consists of 2 nucleotides. In certain embodiments, the additional nucleotide sequence is reminiscent to the loop or a fragment thereof (e.g., one, two, three, or four nucleotides at the 3′ end of the loop) in a crRNA of a corresponding single guide CRISPR-Cas system. It is understood that an additional nucleotide sequence 5′ to the targeter stem sequence can be dispensable. Accordingly, in certain embodiments, the targeter nucleic acid does not comprise any additional nucleotide 5′ to the targeter stem sequence.

In certain embodiments, the targeter nucleic acid or the single guide nucleic acid further comprises an additional nucleotide sequence containing one or more nucleotides at the 3′ end that does not hybridize with the target nucleotide sequence. The additional nucleotide sequence may protect the targeter nucleic acid from degradation by 3′-5′ exonuclease. In certain embodiments, the additional nucleotide sequence is no more than 100 nucleotides in length. In certain embodiments, the additional nucleotide sequence is no more than 90, 80, 70, 60, 50, 40, 30, 20, or 10 nucleotides in length. In certain embodiments, the additional nucleotide sequence is at least 1, 2, 3, 4, 5, 6, 7, 8, 9, 10, 15, 20, 25, 30, 35, 40, 45, or 50 nucleotides in length. In certain embodiments, the additional nucleotide sequence is 5-100, 5-50, 5-40, 5-30, 5-25, 5-20, 5-15, 5-10, 10-100, 10-50, 10-40, 10-30, 10-25, 10-20, 10-15, 15-100, 15-50, 15-40, 15-30, 15-25, 15-20, 20-100, 20-50, 20-40, 20-30, 20-25, 25-100, 25-50, 25-40, 25-30, 30-100, 30-50, 30-40, 40-100, 40-50, or 50-100 nucleotides in length.

In certain embodiments, the additional nucleotide sequence forms a hairpin with the spacer sequence. Such secondary structure may increase the specificity of guide nucleic acid or the engineered, non-naturally occurring system (see, Kocak et al. (2019) Nat. Biotech. 37:657-66). In certain embodiments, the free energy change during the hairpin formation is greater than or equal to −20 kcal/mol, −15 kcal/mol, −14 kcal/mol, −13 kcal/mol, −12 kcal/mol, −11 kcal/mol, or −10 kcal/mol. In certain embodiments, the free energy change during the hairpin formation is greater than or equal to −5 kcal/mol, −6 kcal/mol, −7 kcal/mol, −8 kcal/mol, −9 kcal/mol, −10 kcal/mol, −11 kcal/mol, −12 kcal/mol, −13 kcal/mol, −14 kcal/mol, or −15 kcal/mol. In certain embodiments, the free energy change during the hairpin formation is in the range of −20 to −10 kcal/mol, −20 to −11 kcal/mol, −20 to −12 kcal/mol, −20 to −13 kcal/mol, −20 to −14 kcal/mol, −20 to −15 kcal/mol, −15 to −10 kcal/mol, −15 to −11 kcal/mol, −15 to −12 kcal/mol, −15 to −13 kcal/mol, −15 to −14 kcal/mol, −14 to −10 kcal/mol, −14 to −11 kcal/mol, −14 to −12 kcal/mol, −14 to −13 kcal/mol, −13 to −10 kcal/mol, −13 to −11 kcal/mol, −13 to −12 kcal/mol, −12 to −10 kcal/mol, −12 to −11 kcal/mol, or −11 to −10 kcal/mol. In other embodiments, the targeter nucleic acid or the single guide nucleic acid does not comprise any nucleotide 3′ to the spacer sequence.

In certain embodiments, the modulator nucleic acid further comprises an additional nucleotide sequence 3′ to the modulator stem sequence. In certain embodiments, the additional nucleotide sequence comprises at least 1 (e.g., at least 2, at least 3, at least 4, at least 5, at least 6, at least 7, at least 8, at least 9, at least 10, at least 15, at least 20, at least 25, at least 30, at least 35, at least 40, at least 45, or at least 50) nucleotides. In certain embodiments, the additional nucleotide sequence consists of 1, 2, 3, 4, 5, 6, 7, 8, 9, 10, 15, 20, 25, 30, 35, 40, 45, or 50 nucleotides. In certain embodiments, the additional nucleotide sequence consists of 1 nucleotide (e.g., uridine). In certain embodiments, the additional nucleotide sequence consists of 2 nucleotides. In certain embodiments, the additional nucleotide sequence is reminiscent to the loop or a fragment thereof (e.g., one, two, three, or four nucleotides at the 5′ end of the loop) in a crRNA of a corresponding single guide CRISPR-Cas system. It is understood that an additional nucleotide sequence 3′ to the modulator stem sequence can be dispensable. Accordingly, in certain embodiments, the modulator nucleic acid does not comprise any additional nucleotide 3′ to the modulator stem sequence.

It is understood that the additional nucleotide sequence 5′ to the targeter stem sequence and the additional nucleotide sequence 3′ to the modulator stem sequence, if present, may interact with each other. For example, although the nucleotide immediately 5′ to the targeter stem sequence and the nucleotide immediately 3′ to the modulator stem sequence do not form a Watson-Crick base pair (otherwise they would constitute part of the targeter stem sequence and part of the modulator stem sequence, respectively), other nucleotides in the additional nucleotide sequence 5′ to the targeter stem sequence and the additional nucleotide sequence 3′ to the modulator stem sequence may form one, two, three, or more base pairs (e.g., Watson-Crick base pairs). Such interaction may affect the stability of a complex comprising the targeter nucleic acid and the modulator nucleic acid.

The stability of a complex comprising a targeter nucleic acid and a modulator nucleic acid can be assessed by the Gibbs free energy change (ΔG) during the formation of the complex, either calculated or actually measured. Where all the predicted base pairing in the complex occurs between a base in the targeter nucleic acid and a base in the modulator nucleic acid, i.e., there is no intra-strand secondary structure, the ΔG during the formation of the complex correlates generally with the ΔG during the formation of a secondary structure within the corresponding single guide nucleic acid. Methods of calculating or measuring the ΔG are known in the art. An exemplary method is RNAfold (rna.tbi.univie.ac.at/cgi-bin/RNAWebSuite/RNAfold.cgi) as disclosed in Gruber et al. (2008) Nucleic Acids Res., 36 (Web Server issue): W70-W74. Unless indicated otherwise, the ΔG values in the present disclosure are calculated by RNAfold for the formation of a secondary structure within a corresponding single guide nucleic acid. In certain embodiments, the ΔG is lower than or equal to −1 kcal/mol, e.g., lower than or equal to −2 kcal/mol, lower than or equal to −3 kcal/mol, lower than or equal to −4 kcal/mol, lower than or equal to −5 kcal/mol, lower than or equal to −6 kcal/mol, lower than or equal to −7 kcal/mol, lower than or equal to −7.5 kcal/mol, or lower than or equal to −8 kcal/mol. In certain embodiments, the ΔG is greater than or equal to −10 kcal/mol, e.g., greater than or equal to −9 kcal/mol, greater than or equal to −8.5 kcal/mol, or greater than or equal to −8 kcal/mol. In certain embodiments, the ΔG is in the range of −10 to −4 kcal/mol. In certain embodiments, the ΔG is in the range of −8 to −4 kcal/mol, −7 to −4 kcal/mol, −6 to −4 kcal/mol, −5 to −4 kcal/mol, −8 to −4.5 kcal/mol, −7 to −4.5 kcal/mol, −6 to −4.5 kcal/mol, or −5 to −4.5 kcal/mol. In certain embodiments, the ΔG is about −8 kcal/mol, −7 kcal/mol, −6 kcal/mol, −5 kcal/mol, −4.9 kcal/mol, −4.8 kcal/mol, −4.7 kcal/mol, −4.6 kcal/mol, −4.5 kcal/mol, −4.4 kcal/mol, −4.3 kcal/mol, −4.2 kcal/mol, −4.1 kcal/mol, or −4 kcal/mol.

It is understood that the ΔG may be affected by a sequence in the targeter nucleic acid that is not within the targeter stem sequence, and/or a sequence in the modulator nucleic acid that is not within the modulator stem sequence. For example, one or more base pairs (e.g., Watson-Crick base pair) between an additional sequence 5′ to the targeter stem sequence and an additional sequence 3′ to the modulator stem sequence may reduce the ΔG, i.e., stabilize the nucleic acid complex. In certain embodiments, the nucleotide immediately 5′ to the targeter stem sequence comprises a uracil or is a uridine, and the nucleotide immediately 3′ to the modulator stem sequence comprises a uracil or is a uridine, thereby forming a nonconventional U-U base pair.

In certain embodiments, the modulator nucleic acid or the single guide nucleic acid comprises a nucleotide sequence referred to herein as a “5′ tail” positioned 5′ to the modulator stem sequence. In a naturally occurring type V-A CRISPR-Cas system, the 5′ tail is a nucleotide sequence positioned 5′ to the stem-loop structure of the crRNA. A 5′ tail in an engineered type V-A CRISPR-Cas system, whether single guide or dual guide, can be reminiscent to the 5′ tail in a corresponding naturally occurring type V-A CRISPR-Cas system.

Without being bound by theory, it is contemplated that the 5′ tail may participate in the formation of the CRISPR-Cas complex. For example, in certain embodiments, the 5′ tail forms a pseudoknot structure with the modulator stem sequence, which is recognized by the Cas protein (see, Yamano et al. (2016) Cell, 165:949). In certain embodiments, the 5′ tail is at least 3 (e.g., at least 4 or at least 5) nucleotides in length. In certain embodiments, the 5′ tail is 3, 4, or 5 nucleotides in length. In certain embodiments, the nucleotide at the 3′ end of the 5′ tail comprises a uracil or is a uridine. In certain embodiments, the second nucleotide in the 5′ tail, the position counted from the 3′ end, comprises a uracil or is a uridine. In certain embodiments, the third nucleotide in the 5′ tail, the position counted from the 3′ end, comprises an adenine or is an adenosine. This third nucleotide may form a base pair (e.g., a Watson-Crick base pair) with a nucleotide 5′ to the modulator stem sequence. Accordingly, in certain embodiments, the modulator nucleic acid comprises a uridine or a uracil-containing nucleotide 5′ to the modulator stem sequence. In certain embodiments, the 5′ tail comprises the nucleotide sequence of 5′-AUU-3′. In certain embodiments, the 5′ tail comprises the nucleotide sequence of 5′-AAUU-3′. In certain embodiments, the 5′ tail comprises the nucleotide sequence of 5′-UAAUU-3′. In certain embodiments, the 5′ tail is positioned immediately 5′ to the modulator stem sequence.

In certain embodiments, the single guide nucleic acid, the targeter nucleic acid, and/or the modulator nucleic acid are designed to reduce the degree of secondary structure other than the hybridization between the targeter stem sequence and the modulator stem sequence. In certain embodiments, no more than about 75%, 50%, 40%, 30%, 25%, 20%, 15%, 10%, 5%, 1%, or fewer of the nucleotides of the single guide nucleic acid other than the targeter stem sequence and the modulator stem sequence participate in self-complementary base pairing when optimally folded. In certain embodiments, no more than about 75%, 50%, 40%, 30%, 25%, 20%, 15%, 10%, 5%, 1%, or fewer of the nucleotides of the targeter nucleic acid and/or the modulator nucleic acid participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106 (1): 23-24; and PA Carr and GM Church, 2009, Nature Biotechnology 27 (12): 1151-62).

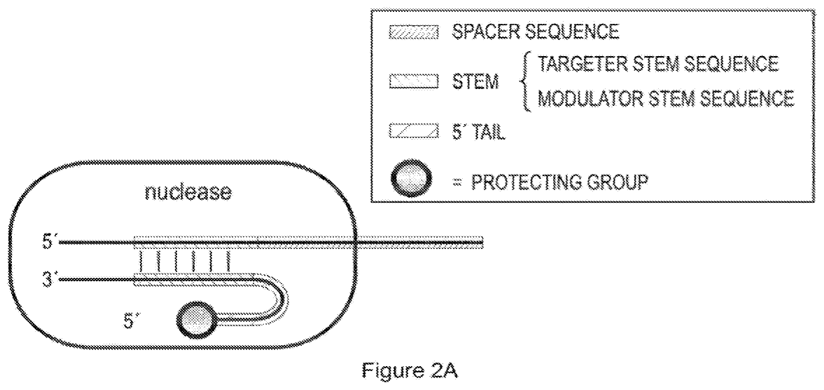

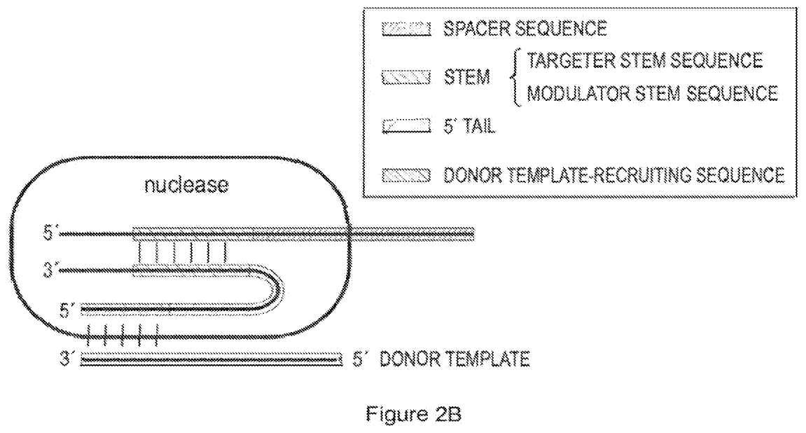

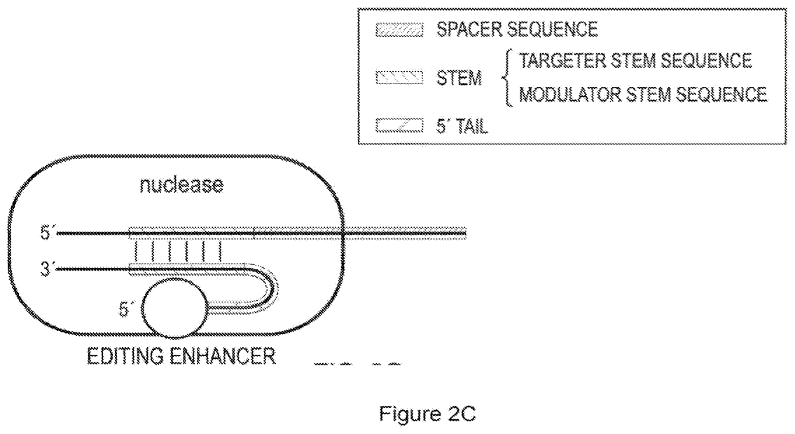

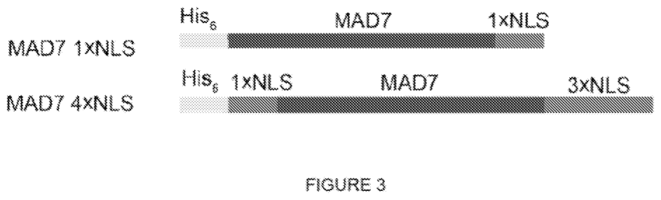

The targeter nucleic acid is directed to a specific target nucleotide sequence, and a donor template can be designed to modify the target nucleotide sequence or a sequence nearby. It is understood, therefore, that association of the single guide nucleic acid, the targeter nucleic acid, or the modulator nucleic acid with a donor template can increase editing efficiency and reduce off-targeting. Accordingly, in certain embodiments, the single guide nucleic acid or the modulator nucleic acid further comprises a donor template-recruiting sequence capable of hybridizing with a donor template (see FIG. 2B). Donor templates are described in the “Donor Templates” subsection of section II infra. The donor template and donor template-recruiting sequence can be designed such that they bear sequence complementarity. In certain embodiments, the donor template-recruiting sequence is at least 90% (e.g., at least 91%, at least 92%, at least 93%, at least 94%, at least 95%, at least 96%, at least 97%, at least 98%, or at least 99%) complementary to at least a portion of the donor template. In certain embodiments, the donor template-recruiting sequence is 100% complementary to at least a portion of the donor template. In certain embodiments, where the donor template comprises an engineered sequence not homologous to the sequence to be repaired, the donor template-recruiting sequence is capable of hybridizing with the engineered sequence in the donor template. In certain embodiments, the donor template-recruiting sequence is at least 20, 25, 30, 35, 40, 45, 50, 55, 60, 65, 70, 75, 80, 85, 90, 95, or 100 nucleotides in length. In certain embodiments, the donor template-recruiting sequence is positioned at or near the 5′ end of the single guide nucleic acid or at or near the 5′ end of the modulator nucleic acid. In certain embodiments, the donor template-recruiting sequence is linked to the 5′ tail, if present, or to the modulator stem sequence, of the single guide nucleic acid or the modulator nucleic acid through an internucleotide bond or a nucleotide linker.

In certain embodiments, the single guide nucleic acid or the modulator nucleic acid further comprises an editing enhancer sequence, which increases the efficiency of gene editing and/or homology-directed repair (HDR) (see FIG. 2C). Exemplary editing enhancer sequences are described in Park et al. (2018) Nat. Commun. 9:3313. In certain embodiments, the editing enhancer sequence is positioned 5′ to the 5′ tail, if present, or 5′ to the single guide nucleic acid or the modulator stem sequence. In certain embodiments, the editing enhancer sequence is 1-50, 4-50, 9-50, 15-50, 25-50, 1-25, 4-25, 9-25, 15-25, 1-15, 4-15, 9-15, 1-9, 4-9, or 1-4 nucleotides in length. In certain embodiments, the editing enhancer sequence is about 1, 2, 3, 4, 5, 6, 7, 8, 9, 10, 15, 20, 25, 30, 35, 40, 45, 50, or 55 nucleotides in length. The editing enhancer sequence is designed to minimize homology to the target nucleotide sequence or any other sequence that the engineered, non-naturally occurring system may be contacted to, e.g., the genome sequence of a cell into which the engineered, non-naturally occurring system is delivered. In certain embodiments, the editing enhancer is designed to minimize the presence of hairpin structure. The editing enhancer can comprise one or more of the chemical modifications disclosed herein.

The single guide nucleic acid, the modulator nucleic acid, and/or the targeter nucleic acid can further comprise a protective nucleotide sequence that prevents or reduces nucleic acid degradation. In certain embodiments, the protective nucleotide sequence is at least 5 (e.g., at least 10, at least 15, at least 20, at least 25, at least 30, at least 35, at least 40, at least 45, or at least 50) nucleotides in length. The length of the protective nucleotide sequence increases the time for an exonuclease to reach the 5′ tail, modulator stem sequence, targeter stem sequence, and/or spacer sequence, thereby protecting these portions of the single guide nucleic acid, the modulator nucleic acid, and/or the targeter nucleic acid from degradation by an exonuclease. In certain embodiments, the protective nucleotide sequence forms a secondary structure, such as a hairpin or a tRNA structure, to reduce the speed of degradation by an exonuclease (see, for example, Wu et al. (2018) Cell. Mol. Life Sci., 75 (19): 3593-3607). Secondary structures can be predicted by methods known in the art, such as the online webserver RNAfold developed at University of Vienna using the centroid structure prediction algorithm (see, Gruber et al. (2008) Nucleic Acids Res., 36: W70). Certain chemical modifications, which may be present in the protective nucleotide sequence, can also prevent or reduce nucleic acid degradation, as disclosed in the “RNA Modifications” subsection infra.

A protective nucleotide sequence is typically located at the 5′ or 3′ end of the single guide nucleic acid, the modulator nucleic acid, and/or the targeter nucleic acid. In certain embodiments, the single guide nucleic acid comprises a protective nucleotide sequence at the 5′ end, at the 3′ end, or at both ends, optionally through a nucleotide linker. In certain embodiments, the modulator nucleic acid comprises a protective nucleotide sequence at the 5′ end, at the 3′ end, or at both ends, optionally through a nucleotide linker. In particular embodiments, the modulator nucleic acid comprises a protective nucleotide sequence at the 5′ end (see FIG. 2A). In certain embodiments, the targeter nucleic acid comprises a protective nucleotide sequence at the 5′ end, at the 3′ end, or at both ends, optionally through a nucleotide linker.

As described above, various nucleotide sequences can be present in the 5′ portion of a single nucleic acid or a modulator nucleic acid, including but not limited to a donor template-recruiting sequence, an editing enhancer sequence, a protective nucleotide sequence, and a linker connecting such sequence to the 5′ tail, if present, or to the modulator stem sequence. It is understood that the functions of donor template recruitment, editing enhancement, protection against degradation, and linkage are not exclusive to each other, and one nucleotide sequence can have one or more of such functions. For example, in certain embodiments, the single guide nucleic acid or the modulator nucleic acid comprises a nucleotide sequence that is both a donor template-recruiting sequence and an editing enhancer sequence. In certain embodiments, the single guide nucleic acid or the modulator nucleic acid comprises a nucleotide sequence that is both a donor template-recruiting sequence and a protective sequence. In certain embodiments, the single guide nucleic acid or the modulator nucleic acid comprises a nucleotide sequence that is both an editing enhancer sequence and a protective sequence. In certain embodiments, the single guide nucleic acid or the modulator nucleic acid comprises a nucleotide sequence that is a donor template-recruiting sequence, an editing enhancer sequence, and a protective sequence. In certain embodiments, the nucleotide sequence 5′ to the 5′ tail, if present, or 5′ to the modulator stem sequence is 1-90, 1-80, 1-70, 1-60, 1-50, 1-40, 1-30, 1-20, 1-10, 10-90, 10-80, 10-70, 10-60, 10-50, 10-40, 10-30, 10-20, 20-90, 20-80, 20-70, 20-60, 20-50, 20-40, 20-30, 30-90, 30-80, 30-70, 30-60, 30-50, 30-40, 40-90, 40-80, 40-70, 40-60, 40-50, 50-90, 50-80, 50-70, 50-60, 60-90, 60-80, 60-70, 70-90, 70-80, or 80-90 nucleotides in length.

In certain embodiments, an engineered, non-naturally occurring system further comprises one or more compounds (e.g., small molecule compounds) that enhance HDR and/or inhibit NHEJ. Exemplary compounds having such functions are described in Maruyama et al. (2015) Nat Biotechnol. 33 (5): 538-42; Chu et al. (2015) Nat Biotechnol. 33 (5): 543-48; Yu et al. (2015) Cell Stem Cell 16 (2): 142-47; Pinder et al. (2015) Nucleic Acids Res. 43 (19): 9379-92; and Yagiz et al. (2019) Commun. Biol. 2:198. In certain embodiments, an engineered, non-naturally occurring system further comprises one or more compounds selected from the group consisting of DNA ligase IV antagonists (e.g., SCR7 compound, Ad4 E1B55K protein, and Ad4 E4orf6 protein), RAD51 agonists (e.g., RS-1), DNA-dependent protein kinase (DNA-PK) antagonists (e.g., NU7441 and KU0060648), B3-adrenergic receptor agonists (e.g., L755507), inhibitors of intracellular protein transport from the ER to the Golgi apparatus (e.g., brefeldin A), and any combinations thereof.

In certain embodiments, an engineered, non-naturally occurring system comprising a targeter nucleic acid and a modulator nucleic acid is tunable or inducible. For example, in certain embodiments, the targeter nucleic acid, the modulator nucleic acid, and/or the Cas protein can be introduced to the target nucleotide sequence at different times, the system becoming active only when all components are present. In certain embodiments, the amounts of the targeter nucleic acid, the modulator nucleic acid, and/or the Cas protein can be titrated to achieve desired efficiency and specificity. In certain embodiments, excess amount of a nucleic acid comprising the targeter stem sequence or the modulator stem sequence can be added to the system, thereby dissociating the complex of the targeter nucleic and modulator nucleic acid and turning off the system.

### C. gNA Modifications

Guide nucleic acids, including a single guide nucleic acid, a targeter nucleic acid, and/or a modulator nucleic acid, may comprise a DNA (e.g., modified DNA), an RNA (e.g., modified RNA), or a combination thereof. In certain embodiments, the single guide nucleic acid comprises a DNA (e.g., modified DNA), an RNA (e.g., modified RNA), or a combination thereof. In certain embodiments, the targeter nucleic acid comprises a DNA (e.g., modified DNA), an RNA (e.g., modified RNA), or a combination thereof. In certain embodiments, the modulator nucleic acid comprises a DNA (e.g., modified DNA), an RNA (e.g., modified RNA), or a combination thereof. Spacer sequences can be presented as DNA sequences by including thymidines (T) rather than uridines (U). It is understood that corresponding RNA sequences and DNA/RNA chimeric sequences are also contemplated. For example, where the spacer sequence is an RNA, its sequence can be derived from a DNA sequence disclosed herein by replacing each T with U. As a result, for the purpose of describing a nucleotide sequence, T and U are used interchangeably herein.

In certain embodiments engineered, non-naturally occurring systems comprising a targeter nucleic acid comprising: a spacer sequence designed to hybridize with a target nucleotide sequence and a targeter stem sequence; and a modulator nucleic acid comprising a modulator stem sequence complementary to the targeter stem sequence, and, optionally, a 5′ sequence, e.g., a tail sequence, wherein, in a single guide nucleic acid the targeter nucleic acid and the modulator nucleic acid are part of a single polynucleotide, and in a dual guide nucleic acid, the targeter nucleic acid and the modulator nucleic acid are separate nucleic acids; modifications can include one or more chemical modifications to one or more nucleotides or internucleotide linkages at or near the 3′ end of the targeter nucleic acid (dual and single gNA), at or near the 5′ end of the targeter nucleic acid (dual gNA), at or near the 3′ end of the modulator nucleic acid (dual gNA), at or near the 5′ end of the modulator nucleic acid (single and dual gNA), or combinations thereof as appropriate for single or dual gNA. In certain embodiments, the Cas nuclease is a type V-A Cas nuclease. Modulator and/or targeter nucleic sequences can include further sequences, as detailed in the Guide Nucleic Acids section, and modifications can be in these further sequences, as appropriate and apparent to one of skill in the art. In embodiments described in this section, below, in certain embodiments, guide nucleic acid is oriented from 5′ at the modulator nucleic acid to 3′ at the modulator stem sequence, and 5′ at the targeter stem sequence to 3′ at the targeter sequence (see, e.g., FIGS. 1A and 1B); in certain embodiments, as appropriate, guide nucleic acid is oriented from 3′ at the modulator nucleic acid to 5′ at the modulator stem sequence, and 3′ at the targeter stem sequence to 5′ at the targeter sequence.

The targeter nucleic acid may comprise a DNA (e.g., modified DNA), an RNA (e.g., modified RNA), or a combination thereof. The modulator nucleic acid may comprise a DNA (e.g., modified DNA), an RNA (e.g., modified RNA), or a combination thereof. In certain embodiments, the targeter nucleic acid is an RNA and the modulator nucleic acid is an RNA. A targeter nucleic acid in the form of an RNA is also called targeter RNA, and a modulator nucleic acid in the form of an RNA is also called modulator RNA. The nucleotide sequences disclosed herein are presented as DNA sequences by including thymidines (T) and/or RNA sequences including uridines (U). It is understood that corresponding DNA sequences, RNA sequences, and DNA/RNA chimeric sequences are also contemplated. For example, where a spacer sequence is presented as a DNA sequence, a nucleic acid comprising this spacer sequence as an RNA can be derived from the DNA sequence disclosed herein by replacing each T with U. As a result, for the purpose of describing a nucleotide sequence, T and U are used interchangeably herein.

In certain embodiments some or all of the gNA is RNA, e.g., a gRNA. In certain embodiments, 5-100%, 10-100%, 20-100%, 30-100%, 40-100%, 50-100%, 60-100%, 70-100%, 80-100%, 90-100%, 95-100%, 99-100%, 99.5-100% of the gNA is gRNA. In certain embodiments, 20%-80%, 20%-70%, 20%-60%, 20%-50%, 20%-40%, 20%-30%, 30%-80%, 30%-70%, 30%-60%, 30%-50%, 30%-40%, 40%-80%, 40%-70%, 40%-60%, 40%-50%, 50%-80%, 50%-70%, 50%-60%, 60%-80%, 60%-70%, or 70%-80% of gNA is RNA. In certain embodiments, 50% of the gNA is RNA. In certain embodiments, 70% of the gNA is RNA. In certain embodiments, 90% of the gNA is RNA. In certain embodiments, 100% of the gNA is RNA, e.g., a gRNA. In further embodiments, the remaining portion of the gNA that is not RNA comprises a modified ribonucleotide, a deoxyribonucleotide, a modified deoxyribonucleotide, or a synthetic, e.g., unnatural nucleotide, for example, not intended to be limiting, threose nucleic acid, locked nucleic acid, peptide nucleic acid, arabinonucleic acid, hexose nucleic acid, among others.

In certain embodiments, the targeter nucleic acid and/or the modulator nucleic acid are RNAs with one or more modifications in a ribose group, one or more modifications in a phosphate group, one or more modifications in a nucleobase, one or more terminal modifications, or a combination thereof. Exemplary modifications are disclosed in U.S. Pat. Nos. 10,900,034 and 10,767,175, U.S. Patent Application Publication No. 2018/0119140, Watts et al. (2008) Drug Discov. Today 13:842-55, and Hendel et al. (2015) NAT. BIOTECHNOL. 33:985.

In certain embodiments, a targeter nucleic acid, e.g., RNA, comprises at least one nucleotide at or near the 3′ end comprising a modification to a ribose, phosphate group, nucleobase, or terminal modification. In certain embodiments, the 3′ end of the targeter nucleic acid comprises the spacer sequence. In certain embodiments, the 3′ end of the targeter nucleic acid comprises the targeter stem sequence. Exemplary modifications are disclosed in Dang et al. (2015) Genome Biol. 16:280, Kocaz et al. (2019) Nature Biotech. 37:657-66, Liu et al. (2019) Nucleic Acids Res. 47 (8): 4169-4180, Schubert et al. (2018) J. Cytokine Biol. 3 (1): 121, Teng et al. (2019) Genome Biol. 20 (1): 15, Watts et al. (2008) Drug Discov. Today 13 (19-20): 842-55, and Wu et al. (2018) Cell Mol. Life. Sci. 75 (19): 3593-607.

Modifications in a ribose group include but are not limited to modifications at the 2′ position or modifications at the 4′ position. For example, in certain embodiments, the ribose comprises 2′-O—C1-4alkyl, such as 2′-O-methyl (2′-OMe, or M). In certain embodiments, the ribose comprises 2′-O—C1-3alkyl-O—C1-3alkyl, such as 2′-methoxyethoxy (2′-O—CH₂CH₂OCH₃) also known as 2′-O-(2-methoxyethyl) or 2′-MOE. In certain embodiments, the ribose comprises 2′-O-allyl. In certain embodiments, the ribose comprises 2′-O-2,4-Dinitrophenol (DNP). In certain embodiments, the ribose comprises 2′-halo, such as 2′-F, 2′-Br, 2′-Cl, or 2′-I. In certain embodiments, the ribose comprises 2′-NH₂. In certain embodiments, the ribose comprises 2′-H (e.g., a deoxynucleotide). In certain embodiments, the ribose comprises 2′-arabino or 2′-F-arabino. In certain embodiments, the ribose comprises 2′-LNA or 2′-ULNA. In certain embodiments, the ribose comprises a 4′-thioribosyl.

Modifications can also include a deoxy group, for example a 2′-deoxy-3′-phosphonoacetate (DP), a 2′-deoxy-3′-thiophosphonoacetate (DSP).

Internucleotide linkage modifications in a phosphate group include but are not limited to a phosphorothioate(S), a chiral phosphorothioate, a phosphorodithioate, a boranophosphonate, a C₁₋₄alkyl phosphonate such as a methylphosphonate, a boranophosphonate, a phosphonocarboxylate such as a phosphonoacetate (P), a phosphonocarboxylate ester such as a phosphonoacetate ester, an amide, a thiophosphonocarboxylate such as a thiophosphonoacetate (SP), a thiophosphonocarboxylate ester such as a thiophosphonoacetate ester, and a 2′,5′-linkage having a phosphodiester or any of the modified phosphates above. Various salts, mixed salts and free acid forms are also included.

Modifications in a nucleobase include but are not limited to 2-thiouracil, 2-thiocytosine, 4-thiouracil, 6-thioguanine, 2-aminoadenine, 2-aminopurine, pseudouracil, hypoxanthine, 7-deazaguanine, 7-deaza-8-azaguanine, 7-deazaadenine, 7-deaza-8-azaadenine, 5-methylcytosine, 5-methyluracil, 5-hydroxymethylcytosine, 5-hydroxymethyluracil, 5,6-dehydrouracil, 5-propynylcytosine, 5-propynyluracil, 5-ethynylcytosine, 5-ethynyluracil, 5-allyluracil, 5-allylcytosine, 5-aminoallyluracil, 5-aminoallyl-cytosine, 5-bromouracil, 5-iodouracil, diaminopurine, difluorotoluene, dihydrouracil, an abasic nucleotide, Z base, P base, Unstructured Nucleic Acid, isoguanine, isocytosine (see, Piccirilli et al. (1990) NATURE, 343: 33), 5-methyl-2-pyrimidine (see, Rappaport (1993) BIOCHEMISTRY, 32:3047), x(A,G,C,T), and y(A,G,C,T).

Terminal modifications include but are not limited to polyethyleneglycol (PEG), hydrocarbon linkers (such as heteroatom (O,S,N)-substituted hydrocarbon spacers; halo-substituted hydrocarbon spacers; keto-, carboxyl-, amido-, thionyl-, carbamoyl-, thionocarbamaoyl-containing hydrocarbon spacers, propanediol), spermine linkers, dyes such as fluorescent dyes (for example, fluoresceins, rhodamines, cyanines), quenchers (for example, dabcyl, BHQ), and other labels (for example biotin, digoxigenin, acridine, streptavidin, avidin, peptides and/or proteins). In certain embodiments, a terminal modification comprises a conjugation (or ligation) of the RNA to another molecule comprising an oligonucleotide (such as deoxyribonucleotides and/or ribonucleotides), a peptide, a protein, a sugar, an oligosaccharide, a steroid, a lipid, a folic acid, a vitamin and/or other molecule. In certain embodiments, a terminal modification incorporated into the RNA is located internally in the RNA sequence via a linker such as 2-(4-butylamidofluorescein)propane-1,3-diol bis(phosphodiester) linker, which is incorporated as a phosphodiester linkage and can be incorporated anywhere between two nucleotides in the RNA.

The modifications disclosed above can be combined in the targeter nucleic acid and/or the modulator nucleic acid that are in the form of RNA. In certain embodiments, the modification in the RNA is selected from the group consisting of incorporation of 2′-O-methyl-3′phosphorothioate (MS), 2′-O-methyl-3′-phosphonoacetate (MP), 2′-O-methyl-3′-thiophosphonoacetate (MSP), 2′-halo-3′-phosphorothioate (e.g., 2′-fluoro-3′-phosphorothioate), 2′-halo-3′-phosphonoacetate (e.g., 2′-fluoro-3′-phosphonoacetate), and 2′-halo-3′-thiophosphonoacetate (e.g., 2′-fluoro-3′-thiophosphonoacetate).

In certain embodiments, modifications can include 2′-O-methyl (M), a phosphorothioate(S), a phosphonoacetate (P), a thiophosphonoacetate (SP), a 2′-O-methyl-3′-phosphorothioate (MS), a 2′-O-methyl-3′-phosphonoacetate (MP), a 2′-O-methyl-3′-thiophosphonoacetate (MSP), a 2′-deoxy-3′-phosphonoacetate (DP), a 2′-deoxy-3′-thiophosphonoacetate (DSP), or a combination thereof, at or near either the 3′ or 5′ end of either the targeter or modulator nucleic acid, as appropriate for single or dual gNA. In certain embodiments, modifications can include either a 5′ or a 3′ propanediol or C3 linker modification.

In certain embodiments, the modification alters the stability of the RNA. In certain embodiments, the modification enhances the stability of the RNA, e.g., by increasing nuclease resistance of the RNA relative to a corresponding RNA without the modification. Stability-enhancing modifications include but are not limited to incorporation of 2′-O-methyl, a 2′-O—C₁₋₄alkyl, 2′-halo (e.g., 2′-F, 2′-Br, 2′-Cl, or 2′-I), 2′MOE, a 2′-O—C₁₋₃alkyl-O—C₁₋₃alkyl, 2′-NH₂, 2′-H (or 2′-deoxy), 2′-arabino, 2′-F-arabino, 4′-thioribosyl sugar moiety, 3′-phosphorothioate, 3′-phosphonoacetate, 3′-thiophosphonoacetate, 3′-methylphosphonate, 3′-boranophosphate, 3′-phosphorodithioate, locked nucleic acid (“LNA”) nucleotide which comprises a methylene bridge between the 2′ and 4′ carbons of the ribose ring, and unlocked nucleic acid (“ULNA”) nucleotide. Such modifications are suitable for use as a protecting group to prevent or reduce degradation of the 5′ sequence, e.g., a tail sequence, modulator stem sequence (dual guide nucleic acids), targeter stem sequence (dual guide nucleic acids), and/or spacer sequence (see, the “Targeter and Modulator nucleic acids” subsection).

In certain embodiments, the modification alters the specificity of the engineered, non-naturally occurring system. In certain embodiments, the modification enhances the specificity of the engineered, non-naturally occurring system, e.g., by enhancing on-target binding and/or cleavage, or reducing off-target binding and/or cleavage, or a combination thereof. Specificity-enhancing modifications include but are not limited to 2-thiouracil, 2-thiocytosine, 4-thiouracil, 6-thioguanine, 2-aminoadenine, and pseudouracil. Within 10, 5, 4, 3, 2, or 1 nucleotide of the 3′ end, for example the 3′ end nucleotide, is modified

In certain embodiments, the modification alters the immunostimulatory effect of the RNA relative to a corresponding RNA without the modification. For example, in certain embodiments, the modification reduces the ability of the RNA to activate TLR7, TLR8, TLR9, TLR3, RIG-I, and/or MDA5.

In certain embodiments, the targeter nucleic acid and/or the modulator nucleic acid comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 modified nucleotides or internucleotide linkages. The modification can be made at one or more positions in the targeter nucleic acid and/or the modulator nucleic acid such that these nucleic acids retain functionality. For example, the modified nucleic acids can still direct the Cas protein to the target nucleotide sequence and allow the Cas protein to exert its effector function. It is understood that the particular modification(s) at a position may be selected based on the functionality of the nucleotide or internucleotide linkage at the position. For example, a specificity-enhancing modification may be suitable for a nucleotide or internucleotide linkage in the spacer sequence, the targeter stem sequence, or the modulator stem sequence. A stability-enhancing modification may be suitable for one or more terminal nucleotides or internucleotide linkages in the targeter nucleic acid and/or the modulator nucleic acid. In certain embodiments, at least 1 (e.g., at least 2, at least 3, at least 4, or at least 5) terminal nucleotides or internucleotide linkages at or near the 5′ end and/or at least 1 (e.g., at least 2, at least 3, at least 4, or at least 5) terminal nucleotides or internucleotide linkages at or near the 3′ end of the targeter nucleic acid are modified. In certain embodiments, 5 or fewer (e.g., 1 or fewer, 2 or fewer, 3 or fewer, or 4 or fewer) terminal nucleotides or internucleotide linkages at or near the 5′ end and/or 5 or fewer (e.g., 1 or fewer, 2 or fewer, 3 or fewer, or 4 or fewer) terminal nucleotides or internucleotide linkages at or near the 3′ end of the targeter nucleic acid are modified. In certain embodiments, at least 1 (e.g., at least 2, at least 3, at least 4, or at least 5) terminal nucleotides or internucleotide linkages at or near the 5′ end and/or at least 1 (e.g., at least 2, at least 3, at least 4, or at least 5) terminal nucleotides or internucleotide linkages at or near the 3′ end of the modulator nucleic acid are modified. In certain embodiments, 5 or fewer (e.g., 1 or fewer, 2 or fewer, 3 or fewer, or 4 or fewer) terminal nucleotides or internucleotide linkages at or near the 5′ end and/or 5 or fewer (e.g., 1 or fewer, 2 or fewer, 3 or fewer, or 4 or fewer) terminal nucleotides or internucleotide linkages at or near the 3′ end of the modulator nucleic acid are modified. Selection of positions for modifications is described in U.S. Pat. Nos. 10,900,034 and 10,767,175. As used in this paragraph, where the targeter or modulator nucleic acid is a combination of DNA and RNA, the nucleic acid as a whole is considered as an RNA, and the DNA nucleotide(s) are considered as modification(s) of the RNA, including a 2′-H modification of the ribose and optionally a modification of the nucleobase.

It is understood that, in dual guide nucleic acid systems the targeter nucleic acid and the modulator nucleic acid, while not in the same nucleic acids, i.e., not linked end-to-end through a traditional internucleotide bond, can be covalently conjugated to each other through one or more chemical modifications introduced into these nucleic acids, thereby increasing the stability of the double-stranded complex and/or improving other characteristics of the system.

### III. COMPOSITION AND METHODS FOR TARGETING, EDITING, AND/OR MODIFYING GENOMIC DNA

An engineered, non-naturally occurring system, such as disclosed herein, can be useful for targeting, editing, and/or modifying a target nucleic acid, such as a DNA (e.g., genomic DNA) in a cell or organism.

The present invention provides a method of cleaving a target nucleic acid (e.g., DNA) comprising the sequence of a preselected target sequence or a portion thereof, the method comprising contacting the target DNA with an engineered, non-naturally occurring system disclosed herein, thereby resulting in cleavage of the target DNA.

In addition, the present invention provides a method of binding a target nucleic acid (e.g., DNA) comprising the sequence of a preselected target sequence or a portion thereof, the method comprising contacting the target DNA with an engineered, non-naturally occurring system disclosed herein, thereby resulting in binding of the system to the target DNA. This method can be useful, e.g., for detecting the presence and/or location of the a preselected target gene, for example, if a component of the system (e.g., the Cas protein) comprises a detectable marker.

In addition, provided are methods of modifying a target nucleic acid (e.g., DNA) comprising the sequence of a preselected target sequence or a portion thereof, or a structure (e.g., protein) associated with the target DNA (e.g., a histone protein in a chromosome), the method comprising contacting the target DNA with an engineered, non-naturally occurring system disclosed herein, wherein the Cas protein comprises an effector domain or is associated with an effector protein, thereby resulting in modification of the target DNA or the structure associated with the target DNA. The modification corresponds to the function of the effector domain or effector protein. Exemplary functions described in the “Cas Proteins” subsection in Section I supra are applicable hereto.

An engineered, non-naturally occurring system can be contacted with the target nucleic acid as a complex. Accordingly, in certain embodiments, a method comprises contacting the target nucleic acid with a CRISPR-Cas complex comprising a targeter nucleic acid, a modulator nucleic acid, and a Cas protein disclosed herein. In certain embodiments, the Cas protein is a type V-A, type V-C, or type V-D Cas protein (e.g., Cas nuclease). In certain embodiments, the Cas protein is a type V-A Cas protein (e.g., Cas nuclease).

In certain embodiments, provided is a method of editing a human genomic sequence at one of a group of preselected target gene loci, the method comprising delivering an engineered, non-naturally occurring system disclosed herein into a human cell, thereby resulting in editing of the genomic sequence at the target gene locus in the human cell. In certain embodiments, provided herein is a method of detecting a human genomic sequence at one of a group of preselected target gene loci, the method comprising delivering the engineered, non-naturally occurring system disclosed herein into a human cell, wherein a component of the system (e.g., the Cas protein) comprises a detectable marker, thereby detecting the target gene locus in the human cell. In certain embodiments, provided herein is a method of modifying a human chromosome at one of a group of preselected target gene loci, the method comprising delivering the engineered, non-naturally occurring system disclosed herein into a human cell, wherein the Cas protein comprises an effector domain or is associated with an effector protein, thereby resulting in modification of the chromosome at the target gene locus in the human cell.

The CRISPR-Cas complex may be delivered to a cell by introducing a pre-formed ribonucleoprotein (RNP) complex into the cell. Alternatively, one or more components of the CRISPR-Cas complex may be expressed in the cell. Exemplary methods of delivery are known in the art and described in, for example, U.S. Pat. Nos. 8,697,359, 10,113,167, 10,570,418, 10,829,787, 11,118,194, and 11,125,739 and U.S. Patent Application Publication Nos. 2015/0344912, 2018/0119140, and 2018/0282763.

It is understood that contacting a DNA (e.g., genomic DNA) in a cell with a CRISPR-Cas complex does not require delivery of all components of the complex into the cell. For example, one or more of the components may be pre-existing in the cell. In certain embodiments, the cell (or a parental/ancestral cell thereof) has been engineered to express the Cas protein, and the single guide nucleic acid (or a nucleic acid comprising a regulatory element operably linked to a nucleotide sequence encoding the single guide nucleic acid), the targeter nucleic acid (or a nucleic acid comprising a regulatory element operably linked to a nucleotide sequence encoding the targeter nucleic acid), and/or the modulator nucleic acid (or a nucleic acid comprising a regulatory element operably linked to a nucleotide sequence encoding the modulator nucleic acid) are delivered into the cell. In certain embodiments, the cell (or a parental/ancestral cell thereof) has been engineered to express the modulator nucleic acid, and the Cas protein (or a nucleic acid comprising a regulatory element operably linked to a nucleotide sequence encoding the Cas protein) and the targeter nucleic acid (or a nucleic acid comprising a regulatory element operably linked to a nucleotide sequence encoding the targeter nucleic acid) are delivered into the cell. In certain embodiments, the cell (or a parental/ancestral cell thereof) has been engineered to express the Cas protein and the modulator nucleic acid, and the targeter nucleic acid (or a nucleic acid comprising a regulatory element operably linked to a nucleotide sequence encoding the targeter nucleic acid) is delivered into the cell.

In certain embodiments, the target DNA is in the genome of a target cell. Accordingly, the present invention also provides a cell comprising the non-naturally occurring system or a CRISPR expression system described herein. In addition, the present invention provides a cell whose genome has been modified by the CRISPR-Cas system or complex disclosed herein.

The target cells can be mitotic or post-mitotic cells from any organism, such as a bacterial cell (e.g., E. coli), an archaeal cell, a cell of a single-cell eukaryotic organism, a plant cell, an algal cell, e.g., Botryococcus braunii, Chlamydomonas reinhardtii, Nannochloropsis gaditana, Chlorella pyrenoidosa, Sargassum patens C. Agardh, or the like, a fungal cell (e.g., a yeast cell, such as S. cervisiae), an animal cell, a cell from an invertebrate animal (e.g. fruit fly, enidarian, echinoderm, nematode, etc.), a cell from a vertebrate animal (e.g., fish, amphibian, reptile, bird, mammal), a cell from a mammal, a cell from a rodent, or a cell from a human. The types of target cells include but are not limited to a stem cell (e.g., an embryonic stem (ES) cell, an induced pluripotent stem (iPS) cell, a germ cell), a somatic cell (e.g., a fibroblast, a hematopoietic cell, a T lymphocyte (e.g., CD8+ T lymphocyte), an NK cell, a neuron, a muscle cell, a bone cell, a hepatocyte, a pancreatic cell), an in vitro or in vivo embryonic cell of an embryo at any stage (e.g., a 1-cell, 2-cell, 4-cell, 8-cell; stage zebrafish embryo). Cells may be from established cell lines or may be primary cells (i.e., cells and cells cultures that have been derived from a subject and allowed to grow in vitro for a limited number of passages of the culture). For example, primary cultures are cultures that may have been passaged within 0 times, 1 time, 2 times, 4 times, 5 times, 10 times, or 15 times, but not enough times to go through the crisis stage. Typically, the primary cell lines are maintained for fewer than 10 passages in vitro. If the cells are primary cells, they may be harvest from an individual by any suitable method. For example, leukocytes may be harvested by apheresis, leukocytapheresis, or density gradient separation, while cells from tissues such as skin, muscle, bone marrow, spleen, liver, pancreas, lung, intestine, or stomach can be harvested by biopsy. The harvested cells may be used immediately, or may be stored under frozen conditions with a cryopreservative and thawed at a later time in a manner as commonly known in the art.

### A. Ribonucleoprotein (RNP) Delivery and “Cas RNA” Delivery

An engineered, non-naturally occurring system disclosed herein can be delivered into a cell by suitable methods known in the art, including but not limited to ribonucleoprotein (RNP) delivery and “Cas RNA” delivery described below.

In certain embodiments, a CRISPR-Cas system including a single guide nucleic acid and a Cas protein, or a CRISPR-Cas system including a targeter nucleic acid, a modulator nucleic acid, and a Cas protein, can be combined into a RNP complex and then delivered into the cell as a pre-formed complex. This method is suitable for active modification of the genetic or epigenetic information in a cell during a limited time period. For example, where the Cas protein has nuclease activity to modify the genomic DNA of the cell, the nuclease activity only needs to be retained for a period of time to allow DNA cleavage, and prolonged nuclease activity may increase off-targeting. Similarly, certain epigenetic modifications can be maintained in a cell once established and can be inherited by daughter cells.

A “ribonucleoprotein” or “RNP,” as used herein, can refer to a complex comprising a nucleoprotein and a ribonucleic acid. A “nucleoprotein” as provided herein can refer to a protein capable of binding a nucleic acid (e.g., RNA, DNA). Where the nucleoprotein binds a ribonucleic acid it can be referred to as “ribonucleoprotein.” The interaction between the ribonucleoprotein and the ribonucleic acid may be direct, e.g., by covalent bond, or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions, or the like). In certain embodiments, the ribonucleoprotein includes an RNA-binding motif non-covalently bound to the ribonucleic acid. For example, positively charged aromatic amino acid residues (e.g., lysine residues) in the RNA-binding motif may form electrostatic interactions with the negative nucleic acid phosphate backbones of the RNA.

To ensure efficient loading of the Cas protein, the single guide nucleic acid, or the combination of the targeter nucleic acid and the modulator nucleic acid, can be provided in excess molar amount (e.g., at least 2 fold, at least 3 fold, at least 4 fold, or at least 5 fold) relative to the Cas protein. In certain embodiments, the targeter nucleic acid and the modulator nucleic acid are annealed under suitable conditions prior to complexing with the Cas protein. In other embodiments, the targeter nucleic acid, the modulator nucleic acid, and the Cas protein are directly mixed together to form an RNP.

A variety of delivery methods can be used to introduce an RNP disclosed herein into a cell. Exemplary delivery methods or vehicles include but are not limited to microinjection, liposomes (see, e.g., U.S. Pat. No. 10,829,787,) such as molecular trojan horses liposomes that delivers molecules across the blood brain barrier (see, Pardridge et al. (2010) Cold Spring Harb. Protoc., doi: 10.1101/pdb.prot5407), immunoliposomes, virosomes, microvesicles (e.g., exosomes and ARMMs), polycations, lipid: nucleic acid conjugates, electroporation, cell permeable peptides (see, U.S. Pat. No. 11,118,194), nanoparticles, nanowires (see, Shalek et al. (2012) Nano Letters, 12:6498), exosomes, and perturbation of cell membrane (e.g., by passing cells through a constriction in a microfluidic system, see, U.S. Pat. No. 11,125,739). Where the target cell is a proliferating cell, the efficiency of RNP delivery can be enhanced by cell cycle synchronization (see, U.S. Pat. No. 10,570,418). In certain embodiments, an RNP is delivered into a cell by electroporation.

In certain embodiments, a CRISPR-Cas system is delivered into a cell in a “approach, i.e., delivering (a) a single guide nucleic acid, or a combination of a targeter nucleic acid and a modulator nucleic acid, and (b) an RNA (e.g., messenger RNA (mRNA)) encoding a Cas protein. The RNA encoding the Cas protein can be translated in the cell and form a complex with the single guide nucleic acid or combination of the targeter nucleic acid and the modulator nucleic acid intracellularly. Similar to the RNP approach, RNAs have limited half-lives in cells, even though stability-increasing modification(s) can be made in one or more of the RNAs. Accordingly, the “Cas RNA” approach is suitable for active modification of the genetic or epigenetic information in a cell during a limited time period, such as DNA cleavage, and has the advantage of reducing off-targeting.

The mRNA can be produced by transcription of a DNA comprising a regulatory element operably linked to a Cas coding sequence. Given that multiple copies of Cas protein can be generated from one mRNA, the single guide nucleic acid, or the targeter nucleic acid and the modulator nucleic acid are generally provided in excess molar amount (e.g., at least 5 fold, at least 10 fold, at least 20 fold, at least 30 fold, at least 50 fold, or at least 100 fold) relative to the mRNA. In certain embodiments, the targeter nucleic acid and the modulator nucleic acid are annealed under suitable conditions prior to delivery into the cells. In other embodiments, the targeter nucleic acid and the modulator nucleic acid are delivered into the cells without annealing in vitro.

A variety of delivery systems can be used to introduce an “Cas RNA” system into a cell. Non-limiting examples of delivery methods or vehicles include microinjection, biolistic particles, liposomes (see, e.g., U.S. Pat. No. 10,829,787) such as molecular trojan horses liposomes that delivers molecules across the blood brain barrier (see, Pardridge et al. (2010) Cold Spring Harb. Protoc., doi: 10.1101/pdb.prot5407), immunoliposomes, virosomes, polycations, lipid: nucleic acid conjugates, electroporation, nanoparticles, nanowires (see, Shalek et al. (2012) Nano Letters, 12:6498), exosomes, and perturbation of cell membrane (e.g., by passing cells through a constriction in a microfluidic system, see, U.S. Pat. No. 11,125,739). Specific examples of the “nucleic acid only” approach by electroporation are described in International (PCT) Publication No. WO 2016/164356.

In certain embodiments, the CRISPR-Cas system is delivered into a cell in the form of (a) a single guide nucleic acid or a combination of a targeter nucleic acid and a modulator nucleic acid, and (b) a DNA comprising a regulatory element operably linked to a Cas coding sequence. The DNA can be provided in a plasmid, viral vector, or any other form described in the “CRISPR Expression Systems” subsection. Such delivery method may result in constitutive expression of Cas protein in the target cell (e.g., if the DNA is maintained in the cell in an episomal vector or is integrated into the genome), and may increase the risk of off-targeting which is undesirable when the Cas protein has nuclease activity. Notwithstanding, this approach is useful when the Cas protein comprises a non-nuclease effector (e.g., a transcriptional activator or repressor). It is also useful for research purposes and for genome editing of plants.

### B. CRISPR Expression Systems

Also provided herein is a nucleic acid comprising a regulatory element operably linked to a nucleotide sequence encoding a guide nucleic acid disclosed herein. In certain embodiments, the nucleic acid comprises a regulatory element operably linked to a nucleotide sequence encoding a single guide nucleic acid; this nucleic acid alone can constitute a CRISPR expression system. In certain embodiments, the nucleic acid comprises a regulatory element operably linked to a nucleotide sequence encoding a targeter nucleic acid. In certain embodiments, the nucleic acid further comprises a nucleotide sequence encoding a modulator nucleic acid, wherein the nucleotide sequence encoding the modulator nucleic acid is operably linked to the same regulatory element as the nucleotide sequence encoding the targeter nucleic acid or a different regulatory element; this nucleic acid alone can constitute a CRISPR expression system.

In addition, the present invention provides a CRISPR expression system comprising: (a) a nucleic acid comprising a first regulatory element operably linked to a nucleotide sequence encoding a targeter nucleic acid and (b) a nucleic acid comprising a second regulatory element operably linked to a nucleotide sequence encoding a modulator nucleic acid.

In certain embodiments, a CRISPR expression system further comprises a nucleic acid comprising a third regulatory element operably linked to a nucleotide sequence encoding a Cas protein, such as a Cas protein disclosed herein. In certain embodiments, the Cas protein is a type V-A, type V-C, or type V-D Cas protein (e.g., Cas nuclease). In certain embodiments, the Cas protein is a type V-A Cas protein (e.g., Cas nuclease).

As used in this context, the term “operably linked” can mean that the nucleotide sequence of interest is linked to the regulatory element in a manner that allows for expression of the nucleotide sequence (e.g., in an in vitro transcription/translation system or in a host cell when the vector is introduced into the host cell).

The nucleic acids of a CRISPR expression system described above may be independently selected from various nucleic acids such as DNA (e.g., modified DNA) and RNA (e.g., modified RNA). In certain embodiments, the nucleic acids comprising a regulatory element operably linked to one or more nucleotide sequences encoding the guide nucleic acids are in the form of DNA. In certain embodiments, the nucleic acid comprising a third regulatory element operably linked to a nucleotide sequence encoding the Cas protein is in the form of DNA. The third regulatory element can be a constitutive or inducible promoter that drives the expression of the Cas protein. In other embodiments, the nucleic acid comprising a third regulatory element operably linked to a nucleotide sequence encoding the Cas protein is in the form of RNA (e.g., mRNA).

Nucleic acids of a CRISPR expression system can be provided in one or more vectors. The term “vector,” as used herein, can refer to a nucleic acid molecule capable of transporting another nucleic acid to which it has been linked. Conventional viral and non-viral based gene transfer methods can be used to introduce nucleic acids in cells, such as prokaryotic cells, eukaryotic cells, mammalian cells, or target tissues. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. Gene therapy procedures are known in the art and disclosed in Van Brunt (1988) BIOTECHNOLOGY, 6:1149; Anderson (1992) SCIENCE, 256:808; Nabel & Feigner (1993) TIBTECH, 11:211; Mitani & Caskey (1993) TIBTECH, 11:162; Dillon (1993) TIBTECH, 11:167; Miller (1992) NATURE, 357:455; Vigne, (1995) RESTORATIVE NEUROLOGY AND NEUROSCIENCE, 8:35; Kremer & Perricaudet (1995) BRITISH MEDICAL BULLETIN, 51:31; Haddada et al. (1995) CURRENT TOPICS IN MICROBIOLOGY AND IMMUNOLOGY, 199:297; Yu et al. (1994) GENE THERAPY, 1:13; and Doerfler and Bohm (Eds.) (2012) The Molecular Repertoire of Adenoviruses II: Molecular Biology of Virus-Cell Interactions. In certain embodiments, at least one of the vectors is a DNA plasmid. In certain embodiments, at least one of the vectors is a viral vector (e.g., retrovirus, adenovirus, or adeno-associated virus).

Certain vectors are capable of autonomous replication in a host cell into which they are introduced (e.g., bacterial vectors having a bacterial origin of replication and episomal mammalian vectors). Other vectors (e.g., non-episomal mammalian vectors and replication defective viral vectors) do not autonomously replicate in the host cell. Certain vectors, however, may be integrated into the genome of the host cell and thereby are replicated along with the host genome. A skilled person in the art will appreciate that different vectors may be suitable for different delivery methods and have different host tropism, and will be able to select one or more vectors suitable for the use.

The term “regulatory element,” as used herein, can refer to a transcriptional and/or translational control sequence, such as a promoter, enhancer, transcription termination signal (e.g., polyadenylation signal), internal ribosomal entry sites (IRES), protein degradation signal, or the like, that provide for and/or regulate transcription of a non-coding sequence (e.g., a targeter nucleic acid or a modulator nucleic acid) or a coding sequence (e.g., a Cas protein) and/or regulate translation of an encoded polypeptide. Such regulatory elements are described, for example, in Goeddel, GENE EXPRESSION TECHNOLOGY: METHODS IN ENZYMOLOGY, 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In certain embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (see, Takebe et al. (1988) MOL. CELL. BIOL., 8:466); SV40 enhancer; and the intron sequence between exons 2 and 3 of rabbit β-globin (see, O'Hare et al. (1981) PROC. NATL. ACAD. SCI. USA., 78:1527). It will be appreciated by those skilled in the art that the design of the expression vector can depend on factors such as the choice of the host cell to be transformed, the level of expression desired, etc. A vector can be introduced into host cells to produce transcripts, proteins, or peptides, including fusion proteins or peptides, encoded by nucleic acids as described herein (e.g., CRISPR transcripts, proteins, enzymes, mutant forms thereof, or fusion proteins thereof).

In certain embodiments, the nucleotide sequence encoding the Cas protein is codon optimized for expression in a prokaryotic cell, e.g., E. coli, eukaryotic host cell, e.g., a yeast cell (e.g., S. cerevisiae), a mammalian cell (e.g., a mouse cell, a rat cell, or a human cell), or a plant cell. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (RNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at kazusa.or.jp/codon/ and these tables can be adapted in a number of ways (see, Nakamura et al. (2000) NUCL. ACIDS RES., 28:292). Computer algorithms for codon optimizing a particular sequence for expression in a particular host cell, such as Gene Forge (Aptagen; Jacobus, Pa.), are also available. In certain embodiments, the codon optimization facilitates or improves expression of the Cas protein in the host cell.

### C. Donor Templates

Cleavage of a target nucleotide sequence in the genome of a cell by a CRISPR-Cas system or complex can activate DNA damage pathways, which may rejoin the cleaved DNA fragments by NHEJ or HDR. HDR requires a repair template, either endogenous or exogenous, to transfer the sequence information from the repair template to the target.

In certain embodiments, an engineered, non-naturally occurring system or CRISPR expression system further comprises a donor template. As used herein, the term “donor template” can refer to a nucleic acid designed to serve as a repair template at or near the target nucleotide sequence upon introduction into a cell or organism. In certain embodiments, the donor template is complementary to a polynucleotide comprising the target nucleotide sequence or a portion thereof. When optimally aligned, a donor template may overlap with one or more nucleotides of a target nucleotide sequences (e.g. about or more than about 1, 5, 10, 15, 20, 25, 30, 35, 40, or more nucleotides). The nucleotide sequence of the donor template is typically not identical to the genomic sequence that it replaces. Rather, the donor template may contain one or more substitutions, insertions, deletions, inversions or rearrangements with respect to the genomic sequence, so long as sufficient homology is present to support homology-directed repair. In certain embodiments, the donor template comprises a non-homologous sequence flanked by two regions of homology (i.e., homology arms), such that homology-directed repair between the target DNA region and the two flanking sequences results in insertion of the non-homologous sequence at the target region. In certain embodiments, the donor template comprises a non-homologous sequence 10-100 nucleotides, 50-500 nucleotides, 100-1,000 nucleotides, 200-2,000 nucleotides, or 500-5,000 nucleotides in length positioned between two homology arms.

Generally, the homologous region(s) of a donor template has at least 50% sequence identity to a genomic sequence with which recombination is desired. The homology arms are designed or selected such that they are capable of recombining with the nucleotide sequences flanking the target nucleotide sequence under intracellular conditions. In certain embodiments, where HDR of the non-target strand is desired, the donor template comprises a first homology arm homologous to a sequence 5′ to the target nucleotide sequence and a second homology arm homologous to a sequence 3′ to the target nucleotide sequence. In certain embodiments, the first homology arm is at least 50% (e.g., at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to a sequence 5′ to the target nucleotide sequence. In certain embodiments, the second homology arm is at least 50% (e.g., at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to a sequence 3′ to the target nucleotide sequence. In certain embodiments, when the donor template sequence and a polynucleotide comprising a target nucleotide sequence are optimally aligned, the nearest nucleotide of the donor template is within about 1, 5, 10, 15, 20, 25, 50, 75, 100, 200, 300, 400, 500, 1000, 2000, 3000, 4000, or more nucleotides from the target nucleotide sequence.

In certain embodiments, the donor template further comprises an engineered sequence not homologous to the sequence to be repaired. Such engineered sequence can harbor a barcode and/or a sequence capable of hybridizing with a donor template-recruiting sequence disclosed herein.

In certain embodiments, the donor template further comprises one or more mutations relative to the genomic sequence, wherein the one or more mutations reduce or prevent cleavage, by the same CRISPR-Cas system, of the donor template or of a modified genomic sequence with at least a portion of the donor template sequence incorporated. In certain embodiments, in the donor template, the PAM adjacent to the target nucleotide sequence and recognized by the Cas nuclease is mutated to a sequence not recognized by the same Cas nuclease. In certain embodiments, in the donor template, the target nucleotide sequence (e.g., the seed region) is mutated. In certain embodiments, the one or more mutations are silent with respect to the reading frame of a protein-coding sequence encompassing the mutated sites.

The donor template can be provided to the cell as single-stranded DNA, single-stranded RNA, double-stranded DNA, or double-stranded RNA. It is understood that a CRISPR-Cas system, such as a system disclosed herein, may possess nuclease activity to cleave the target strand, the non-target strand, or both. When HDR of the target strand is desired, a donor template having a nucleic acid sequence complementary to the target strand is also contemplated.

The donor template can be introduced into a cell in linear or circular form. If introduced in linear form, the ends of the donor template may be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends (see, for example, Chang et al. (1987) PROC. NATL. ACAD SCI USA, 84:4959; Nehls et al. (1996) SCIENCE, 272:886; see also the chemical modifications for increasing stability and/or specificity of RNA disclosed supra). Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. As an alternative to protecting the termini of a linear donor template, additional lengths of sequence may be included outside of the regions of homology that can be degraded without impacting recombination.

A donor template can be a component of a vector as described herein, contained in a separate vector, or provided as a separate polynucleotide, such as an oligonucleotide, linear polynucleotide, or synthetic polynucleotide. In certain embodiments, the donor template is a DNA. In certain embodiments, a donor template is in the same nucleic acid as a sequence encoding the single guide nucleic acid, a sequence encoding the targeter nucleic acid, a sequence encoding the modulator nucleic acid, and/or a sequence encoding the Cas protein, where applicable. In certain embodiments, a donor template is provided in a separate nucleic acid. A donor template polynucleotide may be of any suitable length, such as about or at least about 50, 75, 100, 150, 200, 500, 1000, 2000, 3000, 4000, or more nucleotides in length.

A donor template can be introduced into a cell as an isolated nucleic acid. Alternatively, a donor template can be introduced into a cell as part of a vector (e.g., a plasmid) having additional sequences such as, for example, replication origins, promoters and genes encoding antibiotic resistance, that are not intended for insertion into the DNA region of interest. Alternatively, a donor template can be delivered by viruses (e.g., adenovirus, adeno-associated virus (AAV)). In certain embodiments, the donor template is introduced as an AAV, e.g., a pseudotyped AAV. The capsid proteins of the AAV can be selected by a person skilled in the art based upon the tropism of the AAV and the target cell type. For example, in certain embodiments, the donor template is introduced into a hepatocyte as AAV8 or AAV9. In certain embodiments, the donor template is introduced into a hematopoietic stem cell, a hematopoietic progenitor cell, or a T lymphocyte (e.g., CD8⁺ T lymphocyte) as AAV6 or an AAVHSC (see, U.S. Pat. No. 9,890,396). It is understood that the sequence of a capsid protein (VP1, VP2, or VP3) may be modified from a wild-type AAV capsid protein, for example, having at least 50% (e.g., at least 60%, at least 70%, at least 75%, at least 80%, at least 85%, at least 90%, at least 91%, at least 92%, at least 93%, at least 94%, at least 95%, at least 96%, at least 97%, at least 98%, or at least 99%) sequence identity to a wild-type AAV capsid sequence.

The donor template can be delivered to a cell (e.g., a primary cell) by various delivery methods, such as a viral or non-viral method disclosed herein. In certain embodiments, a non-viral donor template is introduced into the target cell as a naked nucleic acid or in complex with a liposome or poloxamer. In certain embodiments, a non-viral donor template is introduced into the target cell by electroporation. In other embodiments, a viral donor template is introduced into the target cell by infection. The engineered, non-naturally occurring system can be delivered before, after, or simultaneously with the donor template (see, International (PCT) Application Publication No. WO 2017/053729). A skilled person in the art will be able to choose proper timing based upon the form of delivery (consider, for example, the time needed for transcription and translation of RNA and protein components) and the half-life of the molecule(s) in the cell. In particular embodiments, where the CRISPR-Cas system including the Cas protein is delivered by electroporation (e.g., as an RNP), the donor template (e.g., as an AAV) is introduced into the cell within 4 hours (e.g., within 1, 2, 3, 4, 5, 6, 7, 8, 9, 10, 11, 12, 13, 14, 15, 16, 17, 18, 19, 20, 25, 30, 35, 40, 45, 50, 55, 60, 90, 120, 150, 180, 210, or 240 minutes) after the introduction of the engineered, non-naturally occurring system.

In certain embodiments, the donor template is conjugated covalently to a modulator nucleic acid. Covalent linkages suitable for this conjugation are known in the art and are described, for example, in U.S. Pat. No. 9,982,278 and Savic et al. (2018) ELIFE 7:e33761. In certain embodiments, the donor template is covalently linked to a modulator nucleic acid (e.g., the 5′ end of the modulator nucleic acid) through an internucleotide bond. In certain embodiments, the donor template is covalently linked to a modulator nucleic acid (e.g., the 5′ end of the modulator nucleic acid) through a linker.

In certain embodiments, the donor template can comprise any nucleic acid chemistry. In certain embodiments, the donor template can comprise DNA and/or RNA nucleotides. In certain embodiments, the donor template can comprise single-stranded DNA, linear single-stranded RNA, linear double-stranded DNA, linear double-stranded RNA, circular single-stranded DNA, circular single-stranded RNA, circular double-stranded DNA, or circular double-stranded RNA. In certain embodiments, the donor template comprises a mutation in a PAM sequence to partially or completely abolish binding of the RNP to the DNA. In certain embodiments, the donor template is present at a concentration of at least 0.05, 0.01, 0.2, 0.3, 0.4, 0.5, 0.6, 0.7, 0.8, 0.9, 1, 1.25, 1.5, 1.75, 2, 3, or 4, and/or no more than 0.01, 0.2, 0.3, 0.4, 0.5, 0.6, 0.7, 0.8, 0.9, 1, 1.25, 1.5, 1.75, 2, 3, 4, or 5 μg μL⁻¹, for example 0.01-5 μg μL⁻¹. In certain embodiments, the donor template comprises one or more promoters. In certain embodiments, the donor template comprises a promoter that shares at least 70%, at least 75%, at least 80%, at least 85%, at least 90%, at least 95%, at least 99.5% sequence identity with any one of SEQ ID NOs: 78-85 of Table 4.

TABLE 4
Promoter sequences

| | SEQ | |
|---|---|---|
| | ID | |
| Name | NO | Sequence |
| CMV | 78 | CGTTACATAACTTACGGTAAATGGCCCGCCTGGCTGACCGCCCAACGACCCCC |
| | | GCCCATTGACGTCAATAATGACGTATGTTCCCATAGTAACGCCAATAGGGACT |
| | | TTCCATTGACGTCAATGGGTGGAGTATTTACGGTAAACTGCCCACTTGGCAGT |
| | | ACATCAAGTGTATCATATGCCAAGTACGCCCCCTATTGACGTCAATGACGGTA |
| | | AATGGCCCGCCTGGCATTATGCCCAGTACATGACCTTATGGGACTTTCCTACT |
| | | TGGCAGTACATCTACGTATTAGTCATCGCTATTACCATGGTGATGCGGTTTTG |
| | | GCAGTACATCAATGGGCGTGGATAGCGGTTTGACTCACGGGGATTTCCAAGTC |
| | | TCCACCCCATTGACGTCAATGGGAGTTTGTTTTGGCACCAAAATCAACGGGAC |
| | | TTTCCAAAATGTCGTAACAACTCCGCCCCATTGACGCAAATGGGCGGTAGGCG |
| | | TGTACGGTGGGAGGTCTATATAAGCAGAGCT |
| SCP | 79 | GTACTTATATAAGGGGGTGGGGGCGCGTTCGTCCTCAGTCGCGATCGAACACT |
| | | CGAGCCGAGCAGACGTGCCTACGGACCG |
| CMV | 80 | CGTTACATAACTTACGGTAAATGGCCCGCCTGGCTGACCGCCCAACGACCCCC |
| e- | | GCCCATTGACGTCAATAATGACGTATGTTCCCATAGTAACGCCAATAGGGACT |
| SCP | | TTCCATTGACGTCAATGGGTGGAGTATTTACGGTAAACTGCCCACTTGGCAGT |
| | | ACATCAAGTGTATCATATGCCAAGTACGCCCCCTATTGACGTCAATGACGGTA |
| | | AATGGCCCGCCTGGCATTATGCCCAGTACATGACCTTATGGGACTTTCCTACT |
| | | TGGCAGTACATCTACGTATTAGTCATCGCTATTACCATGGTACTTATATAAGG |
| | | GGGTGGGGGCGCGTTCGTCCTCAGTCGCGATCGAACACTCGAGCCGAGCAGAC |
| | | GTGCCTACGGACCG |
| CMV | 81 | TCAATATTGGCCATTAGCCATATTATTCATTGGTTATATAGCATAAATCAATA |
| max | | TTGGCTATTGGCCATTGCATACGTTGTATCTATATCATAATATGTACATTTAT |
| | | ATTGGCTCATGTCCAATATGACCGCCATGTTGGCATTGATTATTGACTAGTTA |
| | | TTAATAGTAATCAATTACGGGGTCATTAGTTCATAGCCCATATATGGAGTTCC |
| | | GCGTTACATAACTTACGGTAAATGGCCCGCCTGGCTGACCGCCCAACGACCCC |
| | | CGCCCATTGACGTCAATAATGACGTATGTTCCCATAGTAACGCCAATAGGGAC |
| | | TTTCCATTGACGTCAATGGGTGGAGTATTTACGGTAAACTGCCCACTTGGCAG |
| | | TACATCAAGTGTATCATATGCCAAGTCCGCCCCCTATTGACGTCAATGACGGT |
| | | AAATGGCCCGCCTGGCATTATGCCCAGTACATGACCTTACGGGACTTTCCTAC |
| | | TTGGCAGTACATCTACGTATTAGTCATCGCTATTACCATGGTGATGCGGTTTT |
| | | GGCAGTACACCAATGGGCGTGGATAGCGGTTTGACTCACGGGGATTTCCAAGT |
| | | CTCCACCCCATTGACGTCAATGGGAGTTTGTTTTGGCACCAAAATCAACGGGA |
| | | CTTTCCAAAATGTCGTAATAACCCCGCCCCGTTGACGCAAATGGGCGGTAGGC |
| | | GTGTACGGTGGGAGGTCTATATAAGCAGAGGTCGTTTAGTGAACCGTCAGATC |
| | | ACTAGTAGCTTTATTGCGGTAGTTTATCACAGTTAAATTGCTAACGCAGTCAG |
| | | TGCTCGACTGATCACAGGTAAGTATCAAGGTTACAAGACAGGTTTAAGGAGGC |
| | | CAATAGAAACTGGGCTTGTCGAGACAGAGAAGATTCTTGCGTTTCTGATAGGC |
| | | ACCTATTGGTCTTACTGACATCCACTTTGCCTTTCTCTCCACAGGG |
| JET | 82 | GAATTCGGGCGGAGTTAGGGCGGAGCCAATCAGCGTGCGCCGTTCCGAAAGTT |
| | | GCCTTTTATGGCTGGGCGGAGAATGGGCGGTGAACGCCGATGATTATATAAGG |
| | | ACGCGCCGGGTGTGGCACAGCTAGTTCCGTCGCAGCCGGGATTTGGGTCGCGG |
| | | TTCTTGTTTGTGGATCCCTGTGATCGTCACTTGACA |
| CAG | 83 | ATCTCGACTAGTTATTAATAGTAATCAATTACGGGGTCATTAGTTCATAGCCC |
| | | ATATATGGAGTTCCGCGTTACATAACTTACGGTAAATGGCCCGCCTGGCTGAC |
| | | CGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAAC |
| | | TGCCCACTTGGCAGTACATCAAGTGTATCATATGCCAAGTACGCCCCCTATTG |
| | | ACGTCAATGACGGTAAATGGCCCGCCTGGCATTATGCCCAGTACATGACCTTA |
| | | TGGGACTTTCCTACTTGGCAGTACATCTACGTATTAGTCATCGCTATTACCAT |
| | | GGTCGAGGTGAGCCCCACGTTCTGCTTCACTCTCCCCATCTCCCCCCCCTCCC |
| | | CACCCCCAATTTTGTATTTATTTATTTTTTAATTATTTTGTGCAGCGATGGGG |
| | | GCGGGGGGGGGGGGGGGGCGCGCGCCAGGCGGGGCGGGGCGGGGCGAGGGGCG |
| | | GGGCGGGGCGAGGCGGAGAGGTGCGGCGGCAGCCAATCAGAGCGGCGCGCTCC |
| | | GAAAGTTTCCTTTTATGGCGAGGCGGCGGCGGCGGCGGCCCTATAAAAAGCGA |
| | | AGCGCGCGGCGGGCGGGGAGTCGCTGCGACGCTGCCTTCGCCCCGTGCCCCGC |
| | | TCCGCCGCCGCCTCGCGCCGCCCGCCCCGGCTCTGACTGACCGCGTTACTCCC |
| | | ACAGGTGAGCGGGCGGGACGGCCCTTCTCCTCCGGGCTGTAATTAGCGCTTGG |
| | | TTTAATGACGGCTTGTTTCTTTTCTGTGGCTGCGTGAAAGCCTTGAGGGGCTC |
| | | CGGGAGGGCCCTTTGTGCGGGGGGAGCGGCTCGGGGGGTGCGTGCGTGTGTGT |
| | | GTGCGTGGGGAGCGCCGCGTGCGGCTCCGCGCTGCCCGGCGGCTGTGAGCGCT |
| | | GCGGGCGCGGCGCGGGGCTTTGTGCGCTCCGCAGTGTGCGCGAGGGGAGCGCG |
| | | GCCGGGGGCGGTGCCCCGCGGTGCGGGGGGGGCTGCGAGGGGAACAAAGGCTG |
| | | CGTGCGGGGTGTGTGCGTGGGGGGGTGAGCAGGGGGTGTGGGCGCGTCGGTCG |
| | | GGCTGCAACCCCCCCTGCACCCCCCTCCCCGAGTTGCTGAGCACGGCCCGGCT |
| | | TCGGGTGCGGGGCTCCGTACGGGGCGTGGCGCGGGGCTCGCCGTGCCGGGCGG |
| | | GGGGTGGCGGCAGGTGGGGGTGCCGGGCGGGGCGGGGCCGCCTCGGGCCGGGG |
| | | AGGGCTCGGGGGAGGGGCGCGGCGGCCCCCGGAGCGCCGGCGGCTGTCGAGGC |
| | | GCGGCGAGCCGCAGCCATTGCCTTTTATGGTAATCGTGCGAGAGGGCGCAGGG |
| | | ACTTCCTTTGTCCCAAATCTGTGCGGAGCCGAAATCTGGGAGGCGCCGCCGCA |
| | | CCCCCTCTAGCGGGCGCGGGGCGAAGCGGTGCGGCGCCGGCAGGAAGGAAATG |
| | | GGCGGGGAGGGCCTTCGTGCGTCGCCGCGCCGCCGTCCCCTTCTCCCTCTCCA |
| | | GCCTCGGGGCTGTCCGCGGGGGGACGGCTGCCTTCGGGGGGGACGGGGCAGGG |
| | | CGGGGTTCGGCTTCTGGCGTGTGACCGGCGGCTCTAGAGCCTCTGCTAACCAT |
| | | GTTCATGCCTTCTTCTTTTTCCTACAGCTCCTGGGCAACGTGCTGGTTATTGT |
| | | GCTGTCTCATCATTTTGGCAAAGAATT |
| PGK | 84 | GGGGTTGGGGTTGCGCCTTTTCCAAGGCAGCCCTGGGTTTGCGCAGGGACGCG |
| | | GCTGCTCTGGGCGTGGTTCCGGGAAACGCAGCGGCGCCGACCCTGGGTCTCGC |
| | | ACATTCTTCACGTCCGTTCGCAGCGTCACCCGGATCTTCGCCGCTACCCTTGT |
| | | GGGCCCCCCGGCGACGCTTCCTGCTCCGCCCCTAAGTCGGGAAGGTTCCTTGC |
| | | GGTTCGCGGCGTGCCGGACGTGACAAACGGAAGCCGCACGTCTCACTAGTACC |
| | | CTCGCAGACGGACAGCGCCAGGGAGCAATGGCAGCGCGCCGACCGCGATGGGC |
| | | TGTGGCCAATAGCGGCTGCTCAGCAGGGCGCGCCGAGAGCAGCGGCCGGGAAG |
| | | GGGCGGTGCGGGAGGCGGGGTGTGGGGCGGTAGTGTGGGCCCTGTTCCTGCCC |
| | | GCGCGGTGTTCCGCATTCTGCAAGCCTCCGGAGCGCACGTCGGCAGTCGGCTC |
| | | CCTCGTTGACCGAATCACCGACCTCTCTCCCCAG |
| EF- | 85 | GAATTCAGGCTCCGGTGCCCGTCAGTGGGCAGAGCGCACATCGCCCACAGTCC |
| 1a | | CCGAGAAGTTGGGGGGAGGGGTCGGCAATTGAACCGGTGCCTAGAGAAGGTGG |
| | | CGCGGGGTAAACTGGGAAAGTGATGTCGTGTACTGGCTCCGCCTTTTTCCCGA |
| | | GGGTGGGGGAGAACCGTATATAAGTGCAGTAGTCGCCGTGAACGTTCTTTTTC |
| | | GCAACGGGTTTGCCGCCAGAACACAGGTAAGTGCCGTGTGTGGTTCCCGCGGG |
| | | CCTGGCCTCTTTACGGGTTATGGCCCTTGCGTGCCTTGAATTACTTCCACCTG |
| | | GCTGCAGTACGTGATTCTTGATCCCGAGCTTCGGGTTGGAAGTGGGTGGGAGA |
| | | GTTCGAGGCCTTGCGCTTAAGGAGCCCCTTCGCCTCGTGCTTGAGTTGAGGCC |
| | | TGGCCTGGGCGCTGGGGCCGCCGCGTGCGAATCTGGTGGCACCTTCGCGCCTG |
| | | TCTCGCTGCTTTCGATAAGTCTCTAGCCATTTAAAATTTTTGATGACCTGCTG |
| | | CGACGCTTTTTTTCTGGCAAGATAGTCTTGTAAATGCGGGCCAAGATCTGCAC |
| | | ACTGGTATTTCGGTTTTTGGGGCCGCGGGCGGCGACGGGGCCCGTGCGTCCCA |
| | | GCGCACATGTTCGGCGAGGCGGGGCCTGCGAGCGCGGCCACCGAGAATCGGAC |
| | | GGGGGTAGTCTCAAGCTGGCCGGCCTGCTCTGGTGCCTGGTCTCGCGCCGCCG |
| | | TGTATCGCCCCGCCCTGGGCGGCAAGGCTGGCCCGGTCGGCACCAGTTGCGTG |
| | | AGCGGAAAGATGGCCGCTTCCCGGCCCTGCTGCAGGGAGCTCAAAATGGAGGA |
| | | CGCGGCGCTCGGGAGAGCGGGCGGGTGAGTCACCCACACAAAGGAAAAGGGCC |
| | | TTTCCGTCCTCAGCCGTCGCTTCATGTGACTCCACGGAGTACCGGGCGCCGTC |
| | | CAGGCACCTCGATTAGTTCTCGAGCTTTTGGAGTACGTCGTCTTTAGGTTGGG |
| | | GGGAGGGGTTTTATGCGATGGAGTTTCCCCACACTGAGTGGGTGGAGACTGAA |
| | | GTTAGGCCAGCTTGGCACTTGATGTAATTCTCCTTGGAATTTGCCCTTTTTGA |
| | | GTTTGGATCTTGGTTCATTCTCAAGCCTCAGACAGTGGTTCAAAGTTTTTTTC |
| | | TTCCATTTCAGGTGTCGTGACATCATTTT |

### D. Efficiency and Specificity

An engineered, non-naturally occurring system can be evaluated in terms of efficiency and/or specificity in nucleic acid targeting, cleavage, or modification.

In certain embodiments, an engineered, non-naturally occurring system has high efficiency. For example, in certain embodiments, at least 1%, at least 1.5%, at least 2%, at least 2.5%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of a population of nucleic acids having the target nucleotide sequence and a cognate PAM, when contacted with the engineered, non-naturally occurring system, is targeted, cleaved, or modified. In certain embodiments, the genomes of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of a population of cells, when the engineered, non-naturally occurring system is delivered into the cells, are targeted, cleaved, or modified.

It has been observed that for a given spacer sequence, the occurrence of on-target events and the occurrence of off-target events are generally correlated. For certain therapeutic purposes, lower on-target efficiency can be tolerated and low off-target frequency is more desirable. For example, when editing or modifying a proliferating cell that will be delivered to a subject and proliferate in vivo, tolerance to off-target events is low. Prior to delivery, it is possible to assess the on-target and off-target events, thereby selecting one or more colonies that have the desired edit or modification and lack any undesired edit or modification. Notwithstanding, the on-target efficiency may need to meet a certain standard to be suitable for therapeutic use. High editing efficiency in a standard CRISPR-Cas system allows tuning of the system, for example, by reducing the binding of the guide nucleic acids to the Cas protein, without losing therapeutic applicability.

In certain embodiments, when a population of nucleic acids having the target nucleotide sequence and a cognate PAM is contacted with the engineered, non-naturally occurring system disclosed herein, the frequency of off-target events (e.g., targeting, cleavage, or modification, depending on the function of the CRISPR-Cas system) is reduced. Methods of assessing off-target events were summarized in Lazzarotto et al. (2018) Nat Protoc. 13 (11): 2615-42, and include discovery of in situ Cas off-targets and verification by sequencing (DISCOVER-seq) as disclosed in Wienert et al. (2019) Science 364 (6437): 286-89; genome-wide unbiased identification of double-stranded breaks (DSBs) enabled by sequencing (GUIDE-seq) as disclosed in Kleinstiver et al. (2016) Nat. Biotech. 34:869-74; circularization for in vitro reporting of cleavage effects by sequencing (CIRCLE-seq) as described in Kocak et al. (2019) Nat. Biotech. 37:657-66. In certain embodiments, the off-target events include targeting, cleavage, or modification at a given off-target locus (e.g., the locus with the highest occurrence of off-target events detected). In certain embodiments, the off-target events include targeting, cleavage, or modification at all the loci with detectable off-target events, collectively.

In certain embodiments, genomic mutations are detected in no more than 0.0001%, 0.0002%, 0.0003%, 0.0004%, 0.0005%, 0.0006%, 0.0007%, 0.0008%, 0.0009%, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, or 5% of the cells at any off-target loci (in aggregate). In certain embodiments, the ratio of the percentage of cells having an on-target event to the percentage of cells having any off-target event (e.g., the ratio of the percentage of cells having an on-target editing event to the percentage of cells having a mutation at any off-target loci) is at least 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000. It is understood that genetic variation may be present in a population of cells, for example, by spontaneous mutations, and such mutations are not included as off-target events.

### E. Multiplexing

The method of targeting, editing, and/or modifying a genomic DNA disclosed herein can be conducted in multiplicity. For example, a library of targeter nucleic acids can be used to target multiple genomic loci; a library of donor templates can also be used to generate multiple insertions, deletions, and/or substitutions. The multiplex assay can be conducted in a screening method wherein each separate cell culture (e.g., in a well of a 96-well plate or a 384-well plate) is exposed to a different guide nucleic acid having a different targeter stem sequence and/or a different donor template. The multiplex assay can also be conducted in a selection method wherein a cell culture is exposed to a mixed population of different guide nucleic acids and/or donor templates, and the cells with desired characteristics (e.g., functionality) are enriched or selected by advantageous survival or growth, resistance to a certain agent, expression of a detectable protein (e.g., a fluorescent protein that is detectable by flow cytometry), etc.

In certain embodiments, the plurality of guide nucleic acids and/or the plurality of donor templates are designed for saturation editing. For example, in certain embodiments, each nucleotide position in a sequence of interest is systematically modified with each of all four traditional bases, A, T, G and C. In other embodiments, at least one sequence in each gene from a pool of genes of interest is modified, for example, according to a CRISPR design algorithm. In certain embodiments, each sequence from a pool of exogenous elements of interest (e.g., protein coding sequences, non-protein coding genes, regulatory elements) is inserted into one or more given loci of the genome.

It is understood that the multiplex methods suitable for the purpose of carrying out a screening or selection method, which is typically conducted for research purposes, may be different from the methods suitable for therapeutic purposes. For example, constitutive expression of certain elements (e.g., a Cas nuclease and/or a guide nucleic acid) may be undesirable for therapeutic purposes due to the potential of increased off-targeting. Conversely, for research purposes, constitutive expression of a Cas nuclease and/or a guide nucleic acid may be desirable. For example, the constitutive expression provides a large window during which other elements can be introduced. When a stable cell line is established for the constitutive expression, the number of exogenous elements that need to be co-delivered into a single cell is also reduced. Therefore, constitutive expression of certain elements can increase the efficiency and reduce the complexity of a screening or selection process. Inducible expression of certain elements of the system disclosed herein may also be used for research purposes given similar advantages. Expression may be induced by an exogenous agent (e.g., a small molecule) or by an endogenous molecule or complex present in a particular cell type (e.g., at a particular stage of differentiation). Methods known in the art, such as those described herein, can be used for constitutively or inducibly expressing one or more elements. For example, the specificity of CRISPR nucleases is at least partially dictated by the uniqueness of the spacer (in combination with spacer sequence's proximity to a requisite PAM) and its off-target score can be calculated with algorithms, such as crispr.mit.edu (Hsu et al. (2013) Nat. Biotech. 31:827-832). The highest possible score is 100, which shows probability for high specificity and few off targets. Because our SHS library targets intergenic regions, the algorithm for gRNA prediction should be able to make alignments with repeated regions and low-complexity sequences.

It is further understood that despite the need to introduce multiple elements—the single guide nucleic acid and the Cas protein; or the targeter nucleic acid, the modulator nucleic acid, and the Cas protein—these elements can be delivered into the cell as a single complex of pre-formed RNP. Therefore, the efficiency of the screening or selection process can also be achieved by pre-assembling a plurality of RNP complexes in a multiplex manner.

In certain embodiments, the method disclosed herein further comprises a step of identifying a guide nucleic acid, a Cas protein, a donor template, or a combination of two or more of these elements from the screening or selection process. A set of barcodes may be used, for example, in the donor template between two homology arms, to facilitate the identification. In specific embodiments, the method further comprises harvesting the population of cells; selectively amplifying a genomic DNA or RNA sample including the target nucleotide sequence(s) and/or the barcodes; and/or sequencing the genomic DNA or RNA sample and/or the barcodes that has been selectively amplified.

In addition, the present invention provides a library comprising a plurality of guide nucleic acids, such as a plurality of guide nucleic acids disclosed herein. In another aspect, the present invention provides a library comprising a plurality of nucleic acids each comprising a regulatory element operably linked to a different guide nucleic acid such as a different guide nucleic acid disclosed herein. These libraries can be used in combination with one or more Cas proteins or Cas-coding nucleic acids, such as disclosed herein, and/or one or more donor templates, such as disclosed herein, for a screening or selection method.

TABLE 5
Spacer sequences

| Name | PAM | SEQ ID NO | Spacer sequence |
|---|---|---|---|
| crCD247_1 | TTTC | 86 | ACCGCGGCCAUCCUGCAGGCA |
| crCD247_2 | TTTC | 87 | UGAGGGAAAGGACAAGAUGAA |
| crCD247_3 | TTTG | 88 | GGAUCCAGCAGGCCAAAGCUC |
| crCD247_4 | TTTC | 89 | CUAGCAGAGAAGGAAGAACCC |
| crCD247_5 | TTTC | 90 | UGUGUUGCAGUUCAGCAGGAG |
| crCD247_6 | CTTC | 91 | CUGAGGGUUCUUCCUUCUCUG |
| crCD247_7 | CTTC | 92 | CCGUUGUCUUUCCUAGCAGAG |
| crCD247_8 | TTTC | 93 | UGCAGUUCCUGCAGAAGAGGG |
| crCD247_9 | CTTC | 94 | UGCAGGAACUGCAGAAAGAUA |
| crCD247_10 | TTTC | 95 | AUCCCAAUCUCACUGUAGGCC |
| crCD247_11 | CTTT | 96 | CAUCCCAAUCUCACUGUAGGC |
| crCD247_12 | TTTT | 97 | CUCAUUUCACUCCCAAACAAC |
| crCD247_13 | TTTC | 98 | UCAUUUCACUCCCAAACAACC |
| crCD247_14 | TTTC | 99 | ACUCCCAAACAACCAGCGCCG |
| crCD247_15 | CTTA | 100 | CGUUAUAGAGCUGGUUCUGGC |
| crCD247_16 | TTTG | 101 | UUUUCUGAUUUGCUUUCACGC |
| crCD247_17 | TTTC | 102 | UGAUUUGCUUUCACGCCAGGG |
| crCD247_18 | TTTG | 103 | CUUUCACGCCAGGGUCUCAGU |
| crCD247_19 | TTTC | 104 | ACGCCAGGGUCUCAGUACAGC |
| crCD247_20 | TTTC | 105 | CGGAGGGUCUACGGCGAGGCU |
| crCD247_21 | TTTC | 106 | UUAUCUGUUAUAGGAGCUCAA |
| crCD247_22 | CTTA | 107 | UCUGUUAUAGGAGCUCAAUCU |
| crCD247_23 | CTTG | 108 | UCCAAAACAUCGUACUCCUCU |
| crCD247_24 | TTTC | 109 | CCCCCAUCUCAGGGUCCCGGC |
| crCD247_25 | TTTG | 110 | GACAAGAGACGUGGCCGGGAC |
| crCD247_26 | TTTC | 111 | UCUCCCUCUAACGUCUUCCCG |
| crCTLA4_1 | TTTG | 112 | CCUGGAGAUGCAUACUCACAC |
| crCTLA4_2 | TTTG | 113 | CAGAAGACAGGGAUGAAGAGA |
| crCTLA4_3 | TTTC | 114 | CACUGGAGGUGCCCGUGCAGA |
| crCTLA4_4 | TTTG | 115 | UGUGUGAGUAUGCAUCUCCAG |
| crCTLA4_5 | TTTC | 116 | AGCGGCACAAGGCUCAGCUGA |
| crCTLA4_6 | CTTG | 117 | UGCCGCUGAAAUCCAAGGCAA |
| crCTLA4_7 | CTTT | 118 | UCCAUGCUAGCAAUGCACGUG |
| crCTLA4_8 | TTTT | 119 | CCAUGCUAGCAAUGCACGUGG |
| crCTLA4_9 | CTTT | 120 | GUGUGUGAGUAUGCAUCUCCA |
| crCTLA4_10 | CTTT | 121 | GCCUGGAGAUGCAUACUCACA |
| crCTLA4_11 | CTTC | 122 | GGCAGGCUGACAGCCAGGUGA |
| crCTLA4_12 | CTTC | 123 | AGUCACCUGGCUGUCAGCCUG |
| crCTLA4_13 | CTTC | 124 | CUAGAUGAUUCCAUCUGCACG |
| crCTLA4_14 | CTTG | 125 | CCUUGGAUUUCAGCGGCACAA |
| crCTLA4_15 | CTTG | 126 | AUUUCCACUGGAGGUGCCCGU |
| crCTLA4_16 | CTTG | 127 | GAUAGUGAGGUUCACUUGAUU |
| crCTLA4_17 | CTTG | 128 | CAGAUGUAGAGUCCCGUGUCC |
| crCTLA4_18 | TTTG | 129 | CUCACCAAUUACAUAAAUCUG |
| crCTLA4_19 | CTTT | 130 | GCUCACCAAUUACAUAAAUCU |
| crCTLA4_20 | CTTT | 131 | GUUUUCUGUUGCAGAUCCAGA |
| crCTLA4_21 | TTTG | 132 | UUUUCUGUUGCAGAUCCAGAA |
| crCTLA4_22 | TTTT | 133 | CUGUUGCAGAUCCAGAACCGU |
| crCTLA4_23 | CTTC | 134 | CUCCUCUGGAUCCUUGCAGCA |
| crCTLA4_24 | CTTG | 135 | CAGCAGUUAGUUCGGGGUUGU |
| crCTLA4_25 | CTTG | 136 | GAUUUCAGCGGCACAAGGCUC |
| crCTLA4_26 | TTTT | 137 | UUUAUAGCUUUCUCCUCACAG |
| crCTLA4_27 | CTTT | 138 | CUCCUCACAGCUGUUUCUUUG |
| crCTLA4_28 | TTTC | 139 | UCCUCACAGCUGUUUCUUUGA |
| crCTLA4_29 | TTTT | 140 | GCUCAAAGAAACAGCUGUGAG |
| crCTLA4_30 | TTTC | 141 | UUUUUGUGUUUGACAGCUAAA |
| crCTLA4_31 | TTTT | 142 | UGUGUUUGACAGCUAAAGAAA |
| crCTLA4_32 | TTTG | 143 | ACAGCUAAAGAAAAGAAGCCC |
| crCTLA4_33 | TTTT | 144 | CACAUAGACCCCUGUUGUAAG |
| crCTLA4_34 | TTTT | 145 | CACAUUCUGGCUCUGUUGGGG |
| crCTLA4_35 | CTTT | 146 | UCACAUUCUGGCUCUGUUGGG |
| crCTLA4_36 | TTTC | 147 | AGCCUUAUUUUAUUCCCAUCA |
| crCTLA4_37 | TTTC | 148 | UCAAUUGAUGGGAAUAAAAUA |
| crCTLA4_38 | TTTT | 149 | UUCUUCUCUUCAUCCCUGUCU |
| crCTLA4_39 | CTTT | 150 | GCAGAAGACAGGGAUGAAGAG |
| crCTLA4_40 | CTTT | 151 | GGCUUUUCCAUGCUAGCAAUG |
| crCTLA4_41 | TTTG | 152 | GCUUUUCCAUGCUAGCAAUGC |
| crLAG3_1 | TTTG | 153 | GGGUGCAUACCUGUCUGGCUG |
| crLAG3_2 | TTTG | 154 | GGUCACCUGGAUCCCUGGGGA |
| crLAG3_3 | TTTC | 155 | UCAGGACCUUGGCUGGAGGCA |
| crLAG3_4 | TTTC | 156 | CCAGCCUUGGCAAUGCCAGCU |
| crLAG3_5 | TTTG | 157 | UGAGGUGACUCCAGUAUCUGG |
| crLAG3_6 | CTTG | 158 | CUGUUUCUGCAGCCGCUUUGG |
| crLAG3_7 | CTTG | 159 | CACAGUGACUGCCAGCCCCCC |
| crLAG3_8 | TTTT | 160 | GAACUGCUCCUUCAGCCGCCC |
| crLAG3_9 | CTTC | 161 | AGCCGCCCUGACCGCCCAGCC |
| crLAG3_10 | TTTC | 162 | CGCUAAGUGGUGAUGGGGGGA |
| crLAG3_11 | CTTT | 163 | CCGCUAAGUGGUGAUGGGGGG |
| crLAG3_12 | CTTA | 164 | GCGGAAAGCUUCCUCUUCCUG |
| crLAG3_13 | CTTG | 165 | GGGCAGGAAGAGGAAGCUUUC |
| crLAG3_14 | CTTC | 166 | CUCUUCCUGCCCCAAGUCAGC |
| crLAG3_15 | CTTC | 167 | AACGUCUCCAUCAUGUAUAAC |
| crLAG3_16 | TTTT | 168 | CUUUUCUCUUCAGGUCUGGAG |
| crLAG3_17 | TTTC | 169 | UGCAGCCGCUUUGGGUGGCUC |
| crLAG3_18 | TTTT | 170 | CUCUUCAGGUCUGGAGCCCCC |
| crLAG3_19 | CTTG | 171 | ACAGUGUACGCUGGAGCAGGU |
| crLAG3_20 | CTTG | 172 | GCAGUGAGGAAAGACCGGGUC |
| crLAG3_21 | TTTC | 173 | CUCACUGCCAAGUGGACUCCU |
| crLAG3_22 | CTTT | 174 | ACCCUUCGACUAGAGGAUGUG |
| crLAG3_23 | TTTA | 175 | CCCUUCGACUAGAGGAUGUGA |
| crLAG3_24 | CTTC | 176 | GACUAGAGGAUGUGAGCCAGG |
| crLAG3_25 | TTTC | 177 | CCACCUGAGGCUGACCUGUGA |
| crLAG3_26 | CTTT | 178 | CCCACCUGAGGCUGACCUGUG |
| crLAG3_27 | CTTC | 179 | UACUCUUUUCAGUGACUCCCA |
| crLAG3_28 | TTTT | 180 | ACCUGGAGCCACCCAAAGCGG |
| crLAG3_29 | TTTT | 181 | CAGUGACUCCCAAAUCCUUUG |
| crLAG3_30 | CTTC | 182 | CCCAGGGAUCCAGGUGACCCA |
| crLAG3_31 | CTTT | 183 | GGGUCACCUGGAUCCCUGGGG |
| crLAG3_32 | CTTT | 184 | GUGAGGUGACUCCAGUAUCUG |
| crLAG3_33 | CTTT | 185 | GUGUGGAGCUCUCUGGACACC |
| crLAG3_34 | TTTG | 186 | UGUGGAGCUCUCUGGACACCC |
| crLAG3_35 | CTTG | 187 | GCUGGAGGCACAGGAGGCCCA |
| crLAG3_36 | TTTT | 188 | GCUCACCUAGUGAAGCCUCUC |
| crLAG3_37 | CTTT | 189 | CCCAGCCUUGGCAAUGCCAGC |
| crLAG3_38 | CTTG | 190 | GCAAUGCCAGCUGUACCAGGG |
| crLAG3_39 | CTTC | 191 | UUGGAGCAGCAGUGUACUUCA |
| crLAG3_40 | CTTC | 192 | ACAGAGCUGUCUAGCCCAGGU |
| crLAG3_41 | CTTT | 193 | CUCCAUAGGUGCCCAACGCUC |
| crLAG3_42 | TTTC | 194 | UCCAUAGGUGCCCAACGCUCU |
| crLAG3_43 | TTTC | 195 | UCAUCCUUGGUGUCCUUUCUC |
| crLAG3_44 | CTTG | 196 | GUGUCCUUUCUCUGCUCCUUU |
| crLAG3_45 | CTTT | 197 | CUCUGCUCCUUUUGGUGACUG |
| crLAG3_46 | CTTC | 198 | UGCGAAGAGCAGGGGUCACUU |
| crLAG3_47 | CTTT | 199 | UGGUGACUGGAGCCUUUGGCU |
| crLAG3_48 | TTTT | 200 | GGUGACUGGAGCCUUUGGCUU |
| crLAG3_49 | CTTT | 201 | GGCUUUCACCUUUGGAGAAGA |
| crLAG3_50 | TTTG | 202 | GCUUUCACCUUUGGAGAAGAC |
| crLAG3_51 | CTTG | 203 | CUCUAAGGCAGAAAAUCGUCU |
| crLAG3_52 | TTTT | 204 | CUGCCUUAGAGCAAGGGAUUC |
| crLAG3_53 | CTTA | 205 | GAGCAAGGGAUUCACCCUCCG |
| crLAG3_54 | TTTC | 206 | CCGCCCAGUGGCCCGCCCGCU |
| crLAG3_55 | CTTC | 207 | UCGCUAUGGCUGCGCCCAGCC |
| crLAG3_56 | TTTA | 208 | UCCUUGCACAGUGACUGCCAG |
| crPDCD1_1 | TTTA | 209 | GCACGAAGCUCUCCGAUGUGU |
| crPDCD1_2 | TTTC | 210 | UCUGCAGGGACAAUAGGAGCC |
| crPDCD1_3 | TTTC | 211 | CAGUGGCGAGAGAAGACCCCG |
| crPDCD1_4 | TTTC | 212 | CUAGCGGAAUGGGCACCUCAU |
| crPDCD1_5 | CTTC | 213 | GUGCUAAACUGGUACCGCAUG |
| crPDCD1_6 | CTTC | 214 | AACCUGACCUGGGACAGUUUC |
| crPDCD1_7 | CTTG | 215 | UCCGUCUGGUUGCUGGGGCUC |
| crPDCD1_8 | CTTC | 216 | CCCGAGGACCGCAGCCAGCCC |
| crPDCD1_9 | CTTC | 217 | CGUGUCACACAACUGCCCAAC |
| crPDCD1_10 | CTTC | 218 | CACAUGAGCGUGGUCAGGGCC |
| crPDCD1_11 | CTTT | 219 | GAUCUGCGCCUUGGGGGCCAG |
| crPDCD1_12 | TTTG | 220 | AUCUGCGCCUUGGGGGCCAGG |
| crPDCD1_13 | CTTG | 221 | GGGGCCAGGGAGAUGGCCCCA |
| crPDCD1_14 | CTTT | 222 | GUGCCCUUCCAGAGAGAAGGG |
| crPDCD1_15 | TTTG | 223 | UGCCCUUCCAGAGAGAAGGGC |
| crPDCD1_16 | TTTC | 224 | CCUUCCGCUCACCUCCGCCUG |
| crPDCD1_17 | CTTC | 225 | CAGAGAGAAGGGCAGAAGUGC |
| crPDCD1_18 | CTTC | 226 | UGCCCUUCUCUCUGGAAGGGC |
| crPDCD1_19 | TTTG | 227 | GAACUGGCCGGCUGGCCUGGG |
| crPDCD1_20 | CTTT | 228 | CUCCUCAAAGAAGGAGGACCC |
| crPDCD1_21 | TTTC | 229 | UCCUCAAAGAAGGAGGACCCC |
| crPDCD1_22 | CTTC | 230 | UCUCGCCACUGGAAAUCCAGC |
| crPDCD1_23 | CTTT | 231 | CCUAGCGGAAUGGGCACCUCA |
| crPDCD1_24 | CTTC | 232 | CGCUCACCUCCGCCUGAGCAG |
| crPDCD1_25 | CTTG | 233 | GCCCCUCUGACCGGCUUCCUU |
| crPDCD1_26 | CTTC | 234 | UCCACUGCUCAGGCGGAGGUG |
| crPDCD1_27 | CTTC | 235 | UCCCCAGCCCUGCUCGUGGUG |
| crPDCD1_28 | CTTC | 236 | GGUCACCACGAGCAGGGCUGG |
| crPDCD1_29 | CTTC | 237 | ACCUGCAGCUUCUCCAACACA |
| crPDCD1_30 | CTTC | 238 | UCCAACACAUCGGAGAGCUUC |
| crPTPN1_1 | TTTA | 239 | CCUGACAGCGAAUCAUAACAU |
| crPTPN1_2 | TTTC | 240 | AUUCCAACUUACCUAACGGAA |
| crPTPN1_3 | TTTC | 241 | UGUGCGCACUGGUGAUGACAA |
| crPTPN11_4 | TTTC | 242 | CAAUCUGCUCACCUGCUUGAG |
| crPTPN11_5 | TTTC | 243 | UUCUAGUUGAUCAUACCAGGG |
| crPTPN11_6 | TTTA | 244 | AUAACUUACCUCAAAUUCUUC |
| crPTPN11_7 | CTTA | 245 | CCUAACGGAAAGUGUGAAGUC |
| crPTPN11_8 | TTTC | 246 | CAGACACUACAACAACAGGAG |
| crPTPN11_9 | TTTA | 247 | GGUGGUUUCAUGGACAUCUCU |
| crPTPN11_10 | TTTC | 248 | CCAGAGAGAUGUCCAUGAAAC |
| crPTPN6_1 | TTTC | 249 | UAUGACCUGUAUGGAGGGGAG |
| crPTPN6_2 | TTTG | 250 | CGACUCUGACAGAGCUGGUGG |
| CrPTPN6_3 | TTTG | 251 | CAGAAGCAGGAGGUGAAGAAC |
| crPTPN6_4 | TTTG | 252 | ACUGCCCCCCACCCAGGCCUG |
| crPTPN6_5 | CTTA | 253 | UGGGCCCUACUCUGUGACCAA |
| crPTPN6_6 | TTTC | 254 | ACCGAGACCUCAGUGGGCUGG |
| crPTPN6_7 | CTTC | 255 | UCUAGGUGGUACCAUGGCCAC |
| crPTPN6_8 | CTTG | 256 | GCCUGCAGCAGCGUCUCUGCC |
| crPTPN6_9 | TTTC | 257 | UUGUGCGUGAGAGCCUCAGCC |
| crPTPN6_10 | CTTC | 258 | GUGCUUUCUGUGCUCAGUGAC |
| crPTPN6_11 | CTTG | 259 | GGCUGGUCACUGAGCACAGAA |
| crPTPN6_12 | CTTT | 260 | CUGUGCUCAGUGACCAGCCCA |
| crPTPN6_13 | TTTC | 261 | UGUGCUCAGUGACCAGCCCAA |
| crPTPN6_14 | CTTG | 262 | AUGUGGGUGACCCUGAGCGGG |
| CrPTPN6 15 | CTTA | 263 | CCUCGCACAUGACCUUGAUGU |
| crPTPN6_16 | TTTG | 264 | GCUCCCCCCAGGGUGGACGCU |
| crPTPN6_17 | CTTG | 265 | AGCAGGGUCUCUGCAUCCAGC |
| crPTPN6_18 | TTTG | 266 | GAGACCUUCGACAGCCUCACG |
| crPTPN6_19 | CTTC | 267 | GACAGCCUCACGGACCUGGUG |
| crPTPN6_20 | TTTC | 268 | AAGAAGACGGGGAUUGAGGAG |
| crPTPN6_21 | CTTC | 269 | UUGUUCAGUUCCAACACUCGG |
| crPTPN6_22 | CTTG | 270 | GCUGUAUCCUCGGACUCCUGC |
| crPTPN6_23 | TTTC | 271 | CCCACCCACAUCUCAGAGUUU |
| crPTPN6_24 | CTTC | 272 | CAGACGCUGGUGCAAGUUCUU |
| crPTPN6_25 | CTTG | 273 | CACCAGCGUCUGGAAGGGCAG |
| crPTPN6_26 | CTTG | 274 | UUCUCUGGCCGCUGCCCUUCC |
| crPTPN6_27 | CTTG | 275 | AUGUAGUUGGCAUUGAUGUAG |
| crPTPN6_28 | CTTG | 276 | CGUCCAGAACCAGCUGCUAGG |
| crPTPN6_29 | CTTC | 277 | UGGCAGAUGGCGUGGCAGGAG |
| crPTPN6_30 | TTTC | 278 | UCCACCUCUCGGGUGGUCAUG |
| crPTPN6_31 | CTTT | 279 | CUCCACCUCUCGGGUGGUCAU |
| crPTPN6_32 | CTTT | 280 | CCAGAACAAAUGCGUCCCAUA |
| crPTPN6_33 | TTTC | 281 | CAGAACAAAUGCGUCCCAUAC |
| crPTPN6_34 | TTTG | 282 | UAUUCGGUUGUGUCAUGCUCC |
| crPTPN6_35 | CTTA | 283 | CAGGUCUCCCCGCUGGACAAU |
| crPTPN6_36 | CTTC | 284 | CUGGCUCGGCCCAGUCGCAAG |
| crPTPN6_37 | CTTA | 285 | GGGAGACCUGAUUCGGGAGAU |
| crPTPN6_38 | CTTC | 286 | CUGGACCAGAUCAACCAGCGG |
| crPTPN6_39 | TTTC | 287 | CUGCCGCUGGUUGAUCUGGUC |
| crPTPN6_40 | CTTT | 288 | CCUGCCGCUGGUUGAUCUGGU |
| crPTPN6_41 | CTTG | 289 | GUGGAGAUGUUCUCCAUGAGC |
| crPTPN6_42 | CTTG | 290 | UACUGCGCCUCCGUCUGCACC |
| crPTPN6_43 | TTTC | 291 | AAUGAACUGGGCGAUGGCCAC |
| crPTPN6_44 | CTTC | 292 | UUCUUAGUGGUUUCAAUGAAC |
| crPTPN6_45 | CTTC | 293 | UCCCCUCCAUACAGGUCAUAG |
| crPTPN6_46 | CTTG | 294 | GAGUCUAGUGCAGGGACCGUG |
| crPTPN6_47 | CTTG | 295 | CCCCCCUGCACCCGGCUGCAG |
| crPTPN6_48 | CTTG | 296 | UGUCUGCAGCCGGGUGCAGGG |
| crPTPN6_49 | TTTC | 297 | UCCUCCCUCUUGUUCUUAGUG |
| crPTPN6_50 | CTTT | 298 | CUCCUCCCUCUUGUUCUUAGU |
| crPTPN6_51 | CTTC | 299 | UUCACUUUCUCCUCCCUCUUG |
| crPTPN6_52 | CTTG | 300 | AGGUGGAUGAUGGUGCCGUCG |
| crPTPN6_53 | CTTC | 301 | CCUGACGCUGCCUUCUCUAGG |
| crTIGIT_1 | TTTC | 302 | AGGCCUUACCUGAGGCGAGGG |
| crTIGIT_2 | TTTT | 303 | GUCCUCCCUCUAGUGGCUGAG |
| crTIGIT_3 | CTTG | 304 | GGGUGGCACAUCUCCCCAUCC |
| crTIGIT_4 | TTTC | 305 | UGCAGAGAAAGGUGGCUCUAU |
| crTIGIT_5 | TTTG | 306 | UAAUGCUGACUUGGGGUGGCA |
| crTIGIT_6 | CTTA | 307 | CCUGAGGCGAGGGGAGCCUGC |
| crTIGIT_7 | CTTG | 308 | AAGGAUGGGGAGAUGUGCCAC |
| crTIGIT_8 | CTTC | 309 | AAGGAUCGAGUGGCCCCAGGU |
| crTIGIT_9 | CTTC | 310 | UGCAUCUAUCACACCUACCCU |
| crTIGIT_10 | TTTC | 311 | UAGGACCUCCAGGAAGAUUCU |
| crTIGIT_11 | CTTT | 312 | CUAGGACCUCCAGGAAGAUUC |
| crTIGIT_12 | CTTG | 313 | CUCCAGCAGGAAUACCUGAGC |
| crTIGIT_13 | CTTG | 314 | GAGCCAUGGCCGCGACGCUGG |
| crTIGIT_14 | TTTC | 315 | UAGUCAACGCGACCACCACGA |
| crTIGIT_15 | CTTT | 316 | CUAGUCAACGCGACCACCACG |
| crTIGIT_16 | TTTG | 317 | UAGUUUGUUUGUUUUUAGAAG |
| crTIGIT_17 | TTTG | 318 | UUUGUUUUUAGAAGAAAGCCC |
| crTIGIT_18 | TTTG | 319 | UUUUUAGAAGAAAGCCCUCAG |
| crTIGIT_19 | TTTT | 320 | UAGAAGAAAGCCCUCAGAAUC |
| crTIGIT_20 | CTTC | 321 | CACAGAAUGGAUUCUGAGGGC |
| crTIGIT_21 | TTTT | 322 | CUCCUGAGGUCACCUUCCACA |
| crTIGIT_22 | CTTC | 323 | CUGGGGGUGAGGGAGCACUGG |
| crTIGIT_23 | CTTC | 324 | UGCCUGGACACAGCUUCCUGG |
| crTIGIT_24 | CTTC | 325 | GUCCUCUUCCCUAGGAAUGAU |
| crTIGIT_25 | CTTC | 326 | UGUAACUCAGGACAUUGAAGU |
| crTIGIT_26 | CTTC | 327 | AAUGUCCUGAGUUACAGAAGC |
| crTIGIT_27 | TTTC | 328 | UAUUGUGCCUGUCAUCAUUCC |
| crTIGIT_28 | TTTC | 329 | UCUGCAGAAAUGUUCCCCGUU |
| crTIGIT_29 | CTTT | 330 | CUCUGCAGAAAUGUUCCCCGU |
| crTIGIT_30 | CTTG | 331 | UGCCGUGGUGGAGGAGAGGUG |
| crTIGIT_31 | CTTC | 332 | UGGCCAUUUGUAAUGCUGACU |
| crTIM3_1 | CTTA | 333 | CUUGUAAGUAGUAGCAGCAGC |
| crTIM3_2 | TTTC | 334 | CAAGGAUGCUUACCACCAGGG |
| crTIM3_3 | CTTG | 335 | UAAGUAGUAGCAGCAGCAGCA |
| crTIM3_4 | CTTA | 336 | CCACCAGGGGACAUGGCCCAG |
| crTIM3_5 | TTTG | 337 | AAUGUGGCAACGUGGUGCUCA |
| crTIM3_6 | CTTT | 338 | UCUUCUGCAAGCUCCAUGUUU |
| crTIM3_7 | CTTT | 339 | GCCCCAGCAGACGGGCACGAG |
| crTIM3_8 | TTTC | 340 | AUCAGUCCUGAGCACCACGUU |
| crTIM3_9 | CTTT | 341 | CAUCAGUCCUGAGCACCACGU |
| crTIM3_10 | TTTA | 342 | GCCAGUAUCUGGAUGUCCAAU |
| crTIM3_11 | TTTG | 343 | CGGAAAUCCCCAUUUAGCCAG |
| crTIM3_12 | CTTT | 344 | GCGGAAAUCCCCAUUUAGCCA |
| crTIM3_13 | TTTC | 345 | CGCAAAGGAGAUGUGUCCCUG |
| crTIM3_14 | TTTG | 346 | GAUCCGGCAGCAGUAGAUCCC |
| crTIM3_15 | TTTT | 347 | UCAUCAUUCAUUAUGCCUGGG |
| crTIM3_16 | TTTT | 348 | CUUCUGCAAGCUCCAUGUUUU |
| crTIM3_17 | CTTC | 349 | AGGUUAAAUUUUUCAUCAUUC |
| crTIM3_18 | TTTG | 350 | AUGACCAACUUCAGGUUAAAU |
| crTIM3_19 | TTTA | 351 | ACCUGAAGUUGGUCAUCAAAC |
| crTIM3_20 | CTTA | 352 | UGUUGUUUCUGACAUUAGCCA |
| crTIM3_21 | TTTC | 353 | UGACAUUAGCCAAGGUCACCC |
| crTIM3_22 | CTTG | 354 | GAAAGGCUGCAGUGAAGUCUC |
| crTIM3_23 | CTTC | 355 | ACUGCAGCCUUUCCAAGGAUG |
| crTIM3_24 | CTTT | 356 | CCAAGGAUGCUUACCACCAGG |
| crTIM3_25 | TTTT | 357 | CACAUCUUCCCUUUGACUGUG |
| crTIM3_26 | TTTT | 358 | UAUAGCAGAGACACAGACACU |
| crTIM3_27 | TTTA | 359 | UAUCAGGGAGGCUCCCCAGUG |
| crTIM3_28 | CTTA | 360 | CUGUUAGAUUUAUAUCAGGGA |
| crTIM3_29 | TTTG | 361 | UGUUUCCAUAGCAAAUAUCCA |
| crTIM3_30 | TTTC | 362 | CAUAGCAAAUAUCCACAUUGG |
| crTIM3_31 | CTTA | 363 | CGGGACUCUGGAGCAACCAUC |
| crTIM3_32 | TTTG | 364 | AAAAUUAAAGCGCCGAAGAUA |
| crTIM3_33 | CTTA | 365 | CAUUUGAAAAUUAAAGCGCCG |
| crTIM3_34 | CTTT | 366 | UGUUUCCCCCUUACUAGGGUA |
| crTIM3_35 | TTTT | 367 | GUUUCCCCCUUACUAGGGUAU |
| crTIM3_36 | CTTT | 368 | GACUGUGUCCUGCUGCUGCUG |
| crTIM3_37 | TTTC | 369 | CCCCUUACUAGGGUAUUCUCA |
| crTIM3_38 | CTTA | 370 | CUAGGGUAUUCUCAUAGCAAA |
| crTIM3_39 | CTTA | 371 | AAUUCUGUAUCUUCUCUUUGC |
| crTIM3_40 | CTTT | 372 | AUUUCCACAGCCUCAUCUCUU |
| crTIM3_41 | TTTA | 373 | UUUCCACAGCCUCAUCUCUUU |
| crTIM3_42 | TTTC | 374 | CACAGCCUCAUCUCUUUGGCC |
| crTIM3_43 | TTTG | 375 | GCCAACCUCCCUCCCUCAGGA |
| crTIM3_44 | TTTG | 376 | CCAAUCCUGAGGGAGGGAGGU |
| crTIM3_45 | TTTT | 377 | CUUCUGAGCGAAUUCCCUCUG |
| crTIM3_46 | CTTC | 378 | AUAUACGUUCUCUUCAAUGGU |
| crTIM3_47 | CTTT | 379 | GGGUUGUCGCUUUGCAAUGCC |
| crTIM3_48 | TTTG | 380 | GGUUGUCGCUUUGCAAUGCCA |
| crTIM3_49 | CTTC | 381 | UCUCUCUAUGCAGGGUCCUCA |
| crTIM3_50 | CTTC | 382 | UACACCCCAGCCGCCCCAGGG |
| crTIM3_51 | TTTG | 383 | CCCCAGCAGACGGGCACGAGG |
| crAAVS1 | TTTC | 384 | TTAGGATGGCCTTCTCCGACG |

### IV. PHARMACEUTICAL COMPOSITIONS

Provided herein is a composition (e.g., pharmaceutical composition) comprising a guide nucleic acid, an engineered, non-naturally occurring system, or a eukaryotic cell, such as a guide nucleic acid, an engineered, non-naturally occurring system, or a eukaryotic cell, disclosed herein. In certain embodiments, the composition comprises an RNP comprising a guide nucleic acid, such as a guide nucleic acid disclosed herein, and a Cas protein (e.g., Cas nuclease). In certain embodiments, the composition comprises a single guide nucleic acid, such as a single guide nucleic acid disclosed herein. In certain embodiments, the composition comprises an RNP comprising the single guide nucleic acid, and a Cas protein (e.g., Cas nuclease). In certain embodiments, the composition comprises an RNP comprising the targeter nucleic acid, the modulator nucleic acid, and a Cas protein (e.g., Cas nuclease). In certain embodiments, the composition comprises a complex of a targeter nucleic acid and a modulator nucleic acid, such as a complex of a targeter nucleic acid and a modulator nucleic acid disclosed herein. In certain embodiments, the composition comprises an RNP comprising the targeter nucleic acid, the modulator nucleic acid, and a Cas protein (e.g., Cas nuclease).

In certain embodiments provided herein is a method of producing a composition, the method comprising incubating a single guide nucleic acid, such as a single guide nucleic acid disclosed herein, with a Cas protein, thereby producing a complex of the single guide nucleic acid and the Cas protein (e.g., an RNP). In certain embodiments, the method further comprises purifying the complex (e.g., the RNP).

In certain embodiments, provided is a method of producing a composition, the method comprising incubating a targeter nucleic acid and a modulator nucleic acid, such as a targeter nucleic acid and a modulator nucleic acid disclosed herein, under suitable conditions, thereby producing a composition (e.g., pharmaceutical composition) comprising a complex of the targeter nucleic acid and the modulator nucleic acid. In certain embodiments, the method further comprises incubating the targeter nucleic acid and the modulator nucleic acid with a Cas protein (e.g., the Cas nuclease that the targeter nucleic acid and the modulator nucleic acid are capable of activating or a related Cas protein), thereby producing a complex of the targeter nucleic acid, the modulator nucleic acid, and the Cas protein (e.g., an RNP). In certain embodiments, the method further comprises purifying the complex (e.g., the RNP).

For therapeutic use, a guide nucleic acid, an engineered, non-naturally occurring system, a CRISPR expression system, or a cell comprising such system or modified by such system disclosed herein is combined with a pharmaceutically acceptable carrier. The term “pharmaceutically acceptable” as used herein can refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit-to-risk ratio.

The term “pharmaceutically acceptable carrier” as used herein includes buffers, carriers, and excipients suitable for use in contact with the tissues of human beings and animals without excessive toxicity, irritation, allergic response, or other problem or complication, commensurate with a reasonable benefit/risk ratio. Pharmaceutically acceptable carriers include any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, e.g., Martin, Remington's Pharmaceutical Sciences, 15th Ed., Mack Publ. Co., Easton, PA (1975). Pharmaceutically acceptable carriers include buffers, solvents, dispersion media, coatings, isotonic and absorption delaying agents, or the like, that are compatible with pharmaceutical administration. The use of such media and agents for pharmaceutically active substances is known in the art.

In certain embodiments, a pharmaceutical composition disclosed herein comprises a salt, e.g., NaCl, MgCl2, KCl, MgSO4, etc.; a buffering agent, e.g., a Tris buffer, N-(2-Hydroxyethyl) piperazine-N′-(2-ethanesulfonic acid) (HEPES), 2-(N-Morpholino) ethanesulfonic acid (MES), MES sodium salt, 3-(N-Morpholino)propanesulfonic acid (MOPS), N-tris [Hydroxymethyl]methyl-3-aminopropanesulfonic acid (TAPS), etc.; a solubilizing agent; a detergent, e.g., a non-ionic detergent such as Tween-20, etc.; a nuclease inhibitor; or the like. For example, in certain embodiments, a subject composition comprises a subject DNA-targeting RNA, e.g., gRNA, and a buffer for stabilizing nucleic acids.

In certain embodiments, a pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants (see, Remington's Pharmaceutical Sciences, 18th ed. (Mack Publishing Company, 1990).

In certain embodiments, a pharmaceutical composition may contain nanoparticles, e.g., polymeric nanoparticles, liposomes, or micelles (See Anselmo et al. (2016) Bioeng. Transl. Med. 1:10-29). In certain embodiment, the pharmaceutical composition comprises an inorganic nanoparticle. Exemplary inorganic nanoparticles include, e.g., magnetic nanoparticles (e.g., Fe3MnO2) or silica. The outer surface of the nanoparticle can be conjugated with a positively charged polymer (e.g., polyethylenimine, polylysine, polyserine) which allows for attachment (e.g., conjugation or entrapment) of payload. In certain embodiment, the pharmaceutical composition comprises an organic nanoparticle (e.g., entrapment of the payload inside the nanoparticle). Exemplary organic nanoparticles include, e.g., SNALP liposomes that contain cationic lipids together with neutral helper lipids which are coated with polyethylene glycol (PEG) and protamine and nucleic acid complex coated with lipid coating. In certain embodiment, the pharmaceutical composition comprises a liposome, for example, a liposome disclosed in International (PCT) Application Publication No. WO 2015/148863.

In certain embodiments, the pharmaceutical composition comprises a targeting moiety to increase target cell binding or update of nanoparticles and liposomes. Exemplary targeting moieties include cell specific antigens, monoclonal antibodies, single chain antibodies, aptamers, polymers, sugars, and cell penetrating peptides. In certain embodiments, the pharmaceutical composition comprises a fusogenic or endosome-destabilizing peptide or polymer.

In certain embodiments, a pharmaceutical composition may contain a sustained- or controlled-delivery formulation. Techniques for formulating sustained- or controlled-delivery means, such as liposome carriers, bio-erodible microparticles or porous beads and depot injections, are also known to those skilled in the art. Sustained-release preparations may include, e.g., porous polymeric microparticles or semipermeable polymer matrices in the form of shaped articles, e.g., films, or microcapsules. Sustained release matrices may include polyesters, hydrogels, polylactides, copolymers of L-glutamic acid and gamma ethyl-L-glutamate, poly (2-hydroxyethyl-methacrylate), ethylene vinyl acetate, or poly-D(−)-3-hydroxybutyric acid. Sustained release compositions may also include liposomes that can be prepared by any of several methods known in the art.

A pharmaceutical composition of the invention can be administered by a variety of methods known in the art. The route and/or mode of administration vary depending upon the desired results. Administration can be intravenous, intramuscular, intraperitoneal, or subcutaneous, or administered proximal to the site of the target. The pharmaceutically acceptable carrier should be suitable for intravenous, intramuscular, subcutaneous, parenteral, spinal or epidermal administration (e.g., by injection or infusion). Depending on the route of administration, the active compound (e.g., the guide nucleic acid, engineered, non-naturally occurring system, or CRISPR expression system disclosed herein) may be coated in a material to protect the compound from the action of acids and other natural conditions that may inactivate the compound.

Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor EL™ (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

Pharmaceutical formulations preferably are sterile. Sterilization can be accomplished by any suitable method, e.g., filtration through sterile filtration membranes. Where the composition is lyophilized, filter sterilization can be conducted prior to or following lyophilization and reconstitution. In certain embodiments, the pharmaceutical composition is lyophilized, and then reconstituted in buffered saline, at the time of administration.

Pharmaceutical compositions of the invention can be prepared in accordance with methods well known and routinely practiced in the art. See, e.g., Remington: The Science and Practice of Pharmacy, Mack Publishing Co., 20th ed., 2000; and Sustained and Controlled Release Drug Delivery Systems, J. R. Robinson, ed., Marcel Dekker, Inc., New York, 1978. Pharmaceutical compositions are preferably manufactured under GMP conditions. Typically, a therapeutically effective dose or efficacious dose of the guide nucleic acid, engineered, non-naturally occurring system, or CRISPR expression system disclosed herein is employed in the pharmaceutical compositions of the invention. The compositions disclosed herein are formulated into pharmaceutically acceptable dosage forms by conventional methods known to those of skill in the art. Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Actual dosage levels of the active ingredients in the pharmaceutical compositions of the invention can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions disclosed herein employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

### V. THERAPEUTIC USES

Guide nucleic acids, engineered, non-naturally occurring systems, and the CRISPR expression systems, e.g., as disclosed herein, are useful for targeting, editing, and/or modifying the genomic DNA in a cell or organism. These guide nucleic acids and systems, as well as a cell comprising one of the systems or a cell whose genome has been modified by one of the systems, can be used to treat a disease or disorder in which modification of genetic or epigenetic information is desirable. Accordingly, provided herein is a method of treating a disease or disorder, the method comprising administering to a subject in need thereof a guide nucleic acid, a non-naturally occurring system, a CRISPR expression system, or a cell disclosed herein.

The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

The terms “treatment”, “treating”, “treat”, “treated”, or the like, as used herein, can refer to obtaining a desired pharmacologic and/or physiologic effect. The effect may be therapeutic in terms of a partial or complete cure for a disease and/or adverse effect attributable to the disease or delaying the disease progression. “Treatment”, as used herein, covers any treatment of a disease in a mammal, e.g., in a human, and includes: (a) inhibiting the disease, i.e., arresting its development; and (b) relieving the disease, i.e., causing regression of the disease. It is understood that a disease or disorder may be identified by genetic methods and treated prior to manifestation of any medical symptom.

For minimization of toxicity and off-target effect, it can be important to control the concentration of the CRISPR-Cas system delivered. Optimal concentrations can be determined by testing different concentrations in a cellular, tissue, or non-human eukaryote animal model and using deep sequencing to analyze the extent of modification at potential off-target genomic loci. The concentration that gives the highest level of on-target modification while minimizing the level of off-target modification is generally selected for ex vivo or in vivo delivery.

It is understood that the guide nucleic acid, the engineered, non-naturally occurring system, and the CRISPR expression system disclosed herein can be used to treat any suitable disease or disorder that can be improved by the system in a cell.

For therapeutic purposes, certain methods disclosed herein is particularly suitable for editing or modifying a proliferating cell, such as a stem cell (e.g., a hematopoietic stem cell), a progenitor cell (e.g., a hematopoietic progenitor cell or a lymphoid progenitor cell), or a memory cell (e.g., a memory T cell). Given that such cell is delivered to a subject and will proliferate in vivo, tolerance to off-target events is low. Prior to delivery, however, it is possible to assess the on-target and off-target events, thereby selecting one or more colonies that have the desired edit or modification and lack any undesired edit or modification. Therefore, lower editing or modifying efficiency can be tolerated for such cell. The engineered, non-naturally occurring system of the present invention has the advantage of increasing or decreasing the efficiency of nucleic acid cleavage by, for example, adjusting the hybridization of dual guide nucleic acids. As a result, it can be used to minimize off-target events when creating genetically engineered proliferating cells.

In certain embodiments, the guide nucleic acid, the engineered, non-naturally occurring system, and/or the CRISPR expression system disclosed herein can be used to engineer an immune cell. Immune cells include but are not limited to lymphocytes (e.g., B lymphocytes or B cells, T lymphocytes or T cells, and natural killer cells), myeloid cells (e.g., monocytes, macrophages, eosinophils, mast cells, basophils, and granulocytes), and the stem and progenitor cells that can differentiate into these cell types (e.g., hematopoietic stem cells, hematopoietic progenitor cells, and lymphoid progenitor cells). The cells can include autologous cells derived from a subject to be treated, or alternatively allogenic cells derived from a donor.

In certain embodiments, the immune cell is a T cell, which can be, for example, a cultured T cell, a primary T cell, a T cell from a cultured T cell line (e.g., Jurkat, SupTi), or a T cell obtained from a mammal, for example, from a subject to be treated. If obtained from a mammal, the T cell can be obtained from numerous sources, including but not limited to blood, bone marrow, lymph node, the thymus, or other tissues or fluids. T cells can also be enriched or purified. The T cell can be any type of T cell and can be of any developmental stage, including but not limited to, CD4⁺/CD8⁺ double positive T cells, CD4⁺ helper T cells (e.g., Th1 and Th2 cells), CD8⁺ T cells (e.g., cytotoxic T cells), tumor infiltrating lymphocytes (TILs), memory T cells (e.g., central memory T cells and effector memory T cells), regulatory T cells, naive T cells, or the like.

In certain embodiments, an immune cell, e.g., a T cell, is engineered to express an exogenous gene. For example, in certain embodiments, an engineered CRISPR system disclosed herein may catalyze DNA cleavage at the gene locus, allowing for site-specific integration of the exogenous gene at the gene locus by HDR.

In certain embodiments, an immune cell, e.g., a T cell, is engineered to express a chimeric antigen receptor (CAR), i.e., the T cell comprises an exogenous nucleotide sequence encoding a CAR. As used herein, the term “chimeric antigen receptor” or “CAR” includes any artificial receptor including an antigen-specific binding moiety and one or more signaling chains derived from an immune receptor. CARs can comprise a single chain fragment variable (scFv) of an antibody specific for an antigen coupled via hinge and transmembrane regions to cytoplasmic domains of T cell signaling molecules, e.g. a T cell costimulatory domain (e.g., from CD28, CD137, OX40, ICOS, or CD27) in tandem with a T cell triggering domain (e.g. from CD3ζ). A T cell expressing a chimeric antigen receptor is referred to as a CAR T cell. Exemplary CAR T cells include CD19 targeted CTL019 cells (see, Grupp et al. (2015) BLOOD, 126:4983), 19-28z cells (see, Park et al. (2015) J. CLIN. ONCOL., 33:7010), and KTE-C19 cells (see, Locke et al. (2015) BLOOD, 126:3991). Additional exemplary CAR T cells are described in U.S. Pat. Nos. 7,446,190, 8,399,645, 8,906,682, 9,181,527, 9,272,002, 9,266,960, 10,253,086, 10,640,569, and 10,808,035, and International (PCT) Publication Nos. WO 2013/142034, WO 2015/120180, WO 2015/188141, WO 2016/120220, and WO 2017/040945. Exemplary approaches to express CARs using CRISPR systems are described in Hale et al. (2017) MOL THER METHODS CLIN DEV., 4:192, MacLeod et al. (2017) MOL THER, 25:949, and Eyquem et al. (2017) NATURE, 543:113.

In certain embodiments, an immune cell, e.g., a T cell, binds an antigen, e.g., a cancer antigen, through an endogenous T cell receptor (TCR). In certain embodiments, an immune cell, e.g., a T cell, is engineered to express an exogenous TCR, e.g., an exogenous naturally occurring TCR or an exogenous engineered TCR. T cell receptors comprise two chains referred to as the α- and β-chains, that combine on the surface of a T cell to form a heterodimeric receptor that can recognize MHC-restricted antigens. Each of α- and β-chain comprises a constant region and a variable region. Each variable region of the α- and β-chains defines three loops, referred to as complementary determining regions (CDRs) known as CDR1, CDR2, and CDR3 that confer the T cell receptor with antigen binding activity and binding specificity.

In certain embodiments, a CAR or TCR binds a cancer antigen selected from B-cell maturation antigen (BCMA), mesothelin, prostate specific membrane antigen (PSMA), prostate stem cell antigen (PSCA), carbonic anhydrase IX (CAIX), carcinoembryonic antigen (CEA), CD5, CD7, CD10, CD19, CD20, CD22, CD30, CD33, CD34, CD38, CD41, CD44, CD49f, CD56, CD70, CD74, CD123, CD133, CD138, epithelial glycoprotein2 (EGP 2), epithelial glycoprotein-40 (EGP-40), epithelial cell adhesion molecule (EpCAM), receptor-type tyrosine-protein kinase (FLT3), folate-binding protein (FBP), fetal acetylcholine receptor (AChR), folate receptor-α and β (FRa and β), Ganglioside G2 (GD2), Ganglioside G3 (GD3), epidermal growth factor receptor 2 (HER-2/ERB2), epidermal growth factor receptor vIII (EGFRvIII), ERB3, ERB4, human telomerase reverse transcriptase (hTERT), Interleukin-13 receptor subunit alpha-2 (IL-13Ra2), K-light chain, kinase insert domain receptor (KDR), Lewis A (CA19.9), Lewis Y (LeY), LI cell adhesion molecule (LICAM), melanoma-associated antigen 1 (melanoma antigen family A1, MAGE-A1), Mucin 16 (MUC-16), Mucin 1 (MUC-1; e.g., a truncated MUC-1), KG2D ligands, cancer-testis antigen NY-ESO-1, oncofetal antigen (h5T4), tumor-associated glycoprotein 72 (TAG-72), vascular endothelial growth factor R2 (VEGF-R2), Wilms tumor protein (WT-1), type 1 tyrosine-protein kinase transmembrane receptor (ROR1), B7-H3 (CD276), B7-H6 (Nkp30), Chondroitin sulfate proteoglycan-4 (CSPG4), DNAX Accessory Molecule (DNAM-1), Ephrin type A Receptor 2 (EpHA2), Fibroblast Associated Protein (FAP), Gp100/HLA-A2, Glypican 3 (GPC3), HA-IH, HERK-V, IL-1 IRa, Latent Membrane Protein 1 (LMP1), Neural cell-adhesion molecule (N-CAM/CD56), and Trail Receptor (TRAIL-R).

Genetic loci suitable for insertion of a CAR- or exogenous TCR-encoding sequence include but are not limited to safe harbor loci (e.g., the AAVS1 locus) TCR subunit loci (e.g., the TCRα constant (TRAC) locus, the TCRβ constant 1 (TRBC1) locus, and the TCRβ constant 2 (TRBC2) locus). It is understood that insertion in the TRAC locus reduces tonic CAR signaling and enhances T cell potency (see, Eyquem et al. (2017) NATURE, 543:113). Furthermore, inactivation of the endogenous TRAC, TRBC1, or TRBC2 gene may reduce a graft-versus-host disease (GVHD) response, thereby allowing use of allogeneic T cells as starting materials for preparation of CAR-T cells. Accordingly, in certain embodiments, an immune cell, e.g., a T cell, is engineered to have reduced expression of an endogenous TCR or TCR subunit, e.g., TRAC, TRBC1, and/or TRBC2. The cell may be engineered to have partially reduced or no expression of the endogenous TCR or TCR subunit. For example, in certain embodiments, the immune cell, e.g., a T cell, is engineered to have less than 80% (e.g., less than 70%, less than 60%, less than 50%, less than 40%, less than 30%, less than 20%, less than 10%, or less than 5%) of the expression of the endogenous TCR or TCR subunit relative to a corresponding unmodified or parental cell. In certain embodiments, the immune cell, e.g., a T cell, is engineered to have no detectable expression of the endogenous TCR or TCR subunit. Exemplary approaches to reduce expression of TCRs using CRISPR systems are described in U.S. Pat. No. 9,181,527, Liu et al. (2017) CELL RES, 27:154, Ren et al. (2017) CLIN CANCER RES, 23:2255, Cooper et al. (2018) LEUKEMIA, 32:1970, and Ren et al. (2017) ONCOTARGET, 8:17002.

It is understood that certain immune cells, such as T cells, also express major histocompatibility complex (MHC) or human leukocyte antigen (HLA) genes, and inactivation of these endogenous gene may reduce an immune response, thereby allowing use of allogeneic T cells as starting materials for preparation of CAR-T cells. Accordingly, in certain embodiments, an immune cell, e.g., a T-cell, is engineered to have reduced expression of one or more endogenous class I or class II MHCs or HLAs (e.g., beta 2-microglobulin (B2M), class II major histocompatibility complex transactivator (CIITA)). The cell may be engineered to have partially reduced or no expression of an endogenous MHC or HLA. For example, in certain embodiments, the immune cell, e.g., a T-cell, is engineered to have less than less than 80% (e.g., less than 70%, less than 60%, less than 50%, less than 40%, less than 30%, less than 20%, less than 10%, or less than 5%) of the expression of endogenous MHC (e.g., B2M, CIITA) relative to a corresponding unmodified or parental cell. In certain embodiments, the immune cell, e.g., a T cell, is engineered to have no detectable expression of an endogenous MHC (e.g., B2M, CIITA). In certain cases, a cell may be engineered to have expression of, e.g., HLA-E and/or HLA-G, in order to avoid attack by natural killer (NK) cells. Exemplary approaches to reduce expression of MHCs using CRISPR systems are described in Liu et al. (2017) CELL RES, 27:154, Ren et al. (2017) CLIN CANCER RES, 23:2255, and Ren et al. (2017) ONCOTARGET, 8:17002.

Other genes that may be inactivated include but are not limited to CD3, CD52, and deoxycytidine kinase (DCK). For example, inactivation of DCK may render the immune cells (e.g., T cells) resistant to purine nucleotide analogue (PNA) compounds, which are often used to compromise the host immune system in order to reduce a GVHD response during an immune cell therapy. In certain embodiments, the immune cell, e.g., a T-cell, is engineered to have less than less than 80% (e.g., less than 70%, less than 60%, less than 50%, less than 40%, less than 30%, less than 20%, less than 10%, or less than 5%) of the expression of endogenous CD52 or DCK relative to a corresponding unmodified or parental cell.

It is understood that the activity of an immune cell (e.g., T cell) may be enhanced by inactivating or reducing the expression of an immune suppressor such as an immune checkpoint protein. Accordingly, in certain embodiments, an immune cell, e.g., a T cell, is engineered to have reduced expression of an immune checkpoint protein. Exemplary immune checkpoint proteins expressed by wild-type T cells include but are not limited to PDCD1 (PD-1), CTLA4, ADORA2A (A2AR), B7-H3, B7-H4, BTLA, KIR, LAG3, HAVCR2 (TIM3), TIGIT, VISTA, PTPN6 (SHP-1), and FAS. The cell may be modified to have partially reduced or no expression of the immune checkpoint protein. For example, in certain embodiments, the immune cell, e.g., a T cell, is engineered to have less than 80% (e.g., less than 70%, less than 60%, less than 50%, less than 40%, less than 30%, less than 20%, less than 10%, or less than 5%) of the expression of the immune checkpoint protein relative to a corresponding unmodified or parental cell. In certain embodiments, the immune cell, e.g., a T cell, is engineered to have no detectable expression of the immune checkpoint protein. Exemplary approaches to reduce expression of immune checkpoint proteins using CRISPR systems are described in International (PCT) Publication No. WO 2017/017184, Cooper et al. (2018) LEUKEMIA, 32:1970, Su et al. (2016) ONCOIMMUNOLOGY, 6: e1249558, and Zhang et al. (2017) FRONT MED, 11:554.

The immune cell can be engineered to have reduced expression of an endogenous gene, e.g., an endogenous genes described above, by gene editing or modification. For example, in certain embodiments, an engineered CRISPR system disclosed herein may result in DNA cleavage at a gene locus, thereby inactivating the targeted gene. In other embodiments, an engineered CRISPR system disclosed herein may be fused to an effector domain (e.g., a transcriptional repressor or histone methylase) to reduce the expression of the target gene.

The immune cell can also be engineered to express an exogenous protein (besides an antigen-binding protein described above) at the locus of a human ADORA2A, B2M, CD52, CIITA, CTLA4, DCK, FAS, HAVCR2, LAG3, PDCD1, PTPN6, TIGIT, TRAC, TRBC1, TRBC2, CARD11, CD247, IL7R, LCK, or PLCG1 gene.

In certain embodiments, an immune cell, e.g., a T cell, is modified to express a dominant-negative form of an immune checkpoint protein. In certain embodiments, the dominant-negative form of the checkpoint inhibitor can act as a decoy receptor to bind or otherwise sequester the natural ligand that would otherwise bind and activate the wild-type immune checkpoint protein. Examples of engineered immune cells, for example, T cells containing dominant-negative forms of an immune suppressor are described, for example, in International (PCT) Publication No. WO 2017/040945.

In certain embodiments, an immune cell, e.g., a T cell, is modified to express a gene (e.g., a transcription factor, a cytokine, or an enzyme) that regulates the survival, proliferation, activity, or differentiation (e.g., into a memory cell) of the immune cell. In certain embodiments, the immune cell is modified to express TET2, FOXO1, IL-12, IL-15, IL-18, IL-21, IL-7, GLUT1, GLUT3, HK1, HK2, GAPDH, LDHA, PDK1, PKM2, PFKFB3, PGK1, ENO1, GYS1, and/or ALDOA. In certain embodiments, the modification is an insertion of a nucleotide sequence encoding the protein operably linked to a regulatory element. In certain embodiments, the modification is a substitution of a single nucleotide polymorphism (SNP) site in the endogenous gene. In certain embodiments, an immune cell, e.g., a T cell, is modified to express a variant of a gene, for example, a variant that has greater activity than the respective wild-type gene. In certain embodiments, the immune cell is modified to express a variant of CARD11, CD247, IL7R, LCK, or PLCG1. For example, certain gain-of-function variants of IL7R were disclosed in Zenatti et al., (2011) NAT. GENET. 43 (10): 932-39. The variant can be expressed from the native locus of the respective wild-type gene by delivering an engineered system described herein for targeting the native locus in combination with a donor template that carries the variant or a portion thereof.

In certain embodiments, an immune cell, e.g., a T cell, is modified to express a protein (e.g., a cytokine or an enzyme) that regulates the microenvironment that the immune cell is designed to migrate to (e.g., a tumor microenvironment). In certain embodiments, the immune cell is modified to express CA9, CA12, a V-ATPase subunit, NHE1, and/or MCT-1.

### A. Gene Therapies

It is understood that the engineered, non-naturally occurring system and CRISPR expression system, e.g., as disclosed herein, can be used to treat a genetic disease or disorder, i.e., a disease or disorder associated with or otherwise mediated by an undesirable mutation in the genome of a subject.

Exemplary genetic diseases or disorders include age-related macular degeneration, adrenoleukodystrophy (ALD), Alagille syndrome, alpha-1-antitrypsin deficiency, argininemia, argininosuccinic aciduria, ataxia (e.g., Friedreich ataxia, spinocerebellar ataxias, ataxia telangiectasia, essential tremor, spastic paraplegia), autism, biliary atresia, biotinidase deficiency, carbamoyl phosphate synthetase I deficiency, carbohydrate deficient glycoprotein syndrome (CDGS), a central nervous system (CNS)-related disorder (e.g., Alzheimer's disease, amyotrophic lateral sclerosis (ALS), canavan disease (CD), ischemia, multiple sclerosis (MS), neuropathic pain, Parkinson's disease), Bloom's syndrome, cancer, Charcot-Marie-Tooth disease (e.g., peroneal muscular atrophy, hereditary motor sensory neuropathy), congenital hepatic porphyria, citrullinemia, Crigler-Najjar syndrome, cystic fibrosis (CF), Dentatorubro-Pallidoluysian Atrophy (DRPLA). diabetes insipidus, Fabry, familial hypercholesterolemia (LDL receptor defect), Fanconi's anemia, fragile X syndrome, a fatty acid oxidation disorder, galactosemia, glucose-6-phosphate dehydrogenase (G6PD), glycogen storage diseases (e.g., type I (glucose-6-phosphatase deficiency, Von Gierke II (alpha glucosidase deficiency, Pompe), III (debrancher enzyme deficiency, Cori), IV (brancher enzyme deficiency, Anderson), V (muscle glycogen phosphorylase deficiency, McArdle), VII (muscle phosphofructokinase deficiency, Tauri), VI (liver phosphorylase deficiency, Hers), IX (liver glycogen phosphorylase kinase deficiency)), hemophilia A (associated with defective factor VIII), hemophilia B (associated with defective factor IX), Huntington's disease, glutaric aciduria, hypophosphatemia, Krabbe, lactic acidosis, Lafora disease, Leber's Congenital Amaurosis, Lesch Nyhan syndrome, a lysosomal storage disease, metachromatic leukodystrophy disease (MLD), mucopolysaccharidosis (MPS) (e.g., Hunter syndrome, Hurler syndrome, Maroteaux-Lamy syndrome, Sanfilippo syndrome, Scheie syndrome, Morquio syndrome, other, MPSI, MPSII, MPSIII, MSIV, MPS 7), a muscular/skeletal disorder (e.g., muscular dystrophy, Duchenne muscular dystrophy), myotonic Dystrophy (DM), neoplasia, N-acetylglutamate synthase deficiency, ornithine transcarbamylase deficiency, phenylketonuria, primary open angle glaucoma, retinitis pigmentosa, schizophrenia, Severe Combined Immune Deficiency (SCID), Spinobulbar Muscular Atrophy (SBMA), sickle cell anemia, Usher syndrome, Tay-Sachs disease, thalassemia (e.g, β-Thalassemia), trinucleotide repeat disorders, tyrosinemia, Wilson's disease, Wiskott-Aldrich syndrome, X-linked chronic granulomatous disease (CGD), X-linked severe combined immune deficiency, and xeroderma pigmentosum.

Additional exemplary genetic diseases or disorders and associated information are available on the world wide web at kumc.edu/gec/support, genome.gov/10001200, and ncbi.nlm.nih.gov/books/NBK22183/. Additional exemplary genetic diseases or disorders, associated genetic mutations, and gene therapy approaches to treat genetic diseases or disorders are described in International (PCT) Publication Nos. WO 2013/126794, WO 2013/163628, WO 2015/048577, WO 2015/070083, WO 2015/089354, WO 2015/134812, WO 2015/138510, WO 2015/148670, WO 2015/148860, WO 2015/148863, WO 2015/153780, WO 2015/153789, and WO 2015/153791, U.S. Pat. Nos. 8,383,604, 8,859,597, 8,956,828, 9,255,130, and 9,273,296, and U.S. Patent Application Publication Nos. 2009/0222937, 2009/0271881, 2010/0229252, 2010/0311124, 2011/0016540, 2011/0023139, 2011/0023144, 2011/0023145, 2011/0023146, 2011/0023153, 2011/0091441, 2012/0159653, and 2013/0145487.

### VI. KITS

It is understood that the guide nucleic acid, the engineered, non-naturally occurring system, the CRISPR expression system, and/or a library disclosed herein can be packaged in a kit suitable for use by a medical provider. Accordingly, in another aspect, the invention provides kits containing any one or more of the elements disclosed in the above systems, libraries, methods, and compositions. In certain embodiments, the kit comprises an engineered, non-naturally occurring system as disclosed herein and instructions for using the kit. The instructions may be specific to the applications and methods described herein. In certain embodiments, one or more of the elements of the system are provided in a solution. In certain embodiments, one or more of the elements of the system are provided in lyophilized form, and the kit further comprises a diluent. Elements may be provided individually or in combinations, and may be provided in any suitable container, such as a vial, a bottle, a tube, or immobilized on the surface of a solid base (e.g., chip or microarray). In certain embodiments, the kit comprises one or more of the nucleic acids and/or proteins described herein. In certain embodiments, the kit provides all elements of the systems of the invention.

In certain embodiments of a kit comprising the engineered, non-naturally occurring dual guide system, the targeter nucleic acid and the modulator nucleic acid are provided in separate containers. In other embodiments, the targeter nucleic acid and the modulator nucleic acid are pre-complexed, and the complex is provided in a single container.

In certain embodiments, the kit comprises a Cas protein or a nucleic acid comprising a regulatory element operably linked to a nucleic acid encoding a Cas protein provided in a separate container. In other embodiments, the kit comprises a Cas protein pre-complexed with the single guide nucleic acid or a combination of the targeter nucleic acid and the modulator nucleic acid, and the complex is provided in a single container.

In certain embodiments, the kit further comprises one or more donor templates provided in one or more separate containers. In certain embodiments, the kit comprises a plurality of donor templates as disclosed herein (e.g., in separate tubes or immobilized on the surface of a solid base such as a chip or a microarray), one or more guide nucleic acids disclosed herein, and optionally a Cas protein or a regulatory element operably linked to a nucleic acid encoding a Cas protein as disclosed herein. Such kits are useful for identifying a donor template that introduces optimal genetic modification in a multiplex assay. The CRISPR expression systems as disclosed herein are also suitable for use in a kit.

In certain embodiments, a kit further comprises one or more reagents and/or buffers for use in a process utilizing one or more of the elements described herein. Reagents may be provided in any suitable container and may be provided in a form that is usable in a particular assay, or in a form that requires addition of one or more other components before use (e.g., in concentrate or lyophilized form). A buffer may be a reaction or storage buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In certain embodiments, the buffer has a pH from about 7 to about 10. In certain embodiments, the kit further comprises a pharmaceutically acceptable carrier. In certain embodiments, the kit further comprises one or more devices or other materials for administration to a subject.

### VII. EMBODIMENTS

In embodiment 1 provided herein is a composition comprising: (a) a nuclease system comprising: (i) a nucleic acid-guided nuclease; and (ii) a guide nucleic acid (gNA) compatible with and capable of binding to and activating the nucleic acid-guided nuclease, wherein the gNA comprises: (1) a targeter nucleic acid comprising a targeter stem sequence and a spacer sequence, wherein the spacer sequence is complementary to a target nucleotide sequence within a target polynucleotide, for example a target polynucleotide of a genome of a human target cell; and (2) a modulator nucleic acid comprising a modulator stem sequence complementary to the targeter stem sequence, and, optionally, a 5′ sequence; and (b) at least one additive that stabilizes the nucleic acid-guided nuclease system. In embodiment 2 provided herein is the composition of embodiment 1, wherein the nuclease comprises a Class 1 nuclease. In embodiment 3 provided herein is the composition of embodiment 1, wherein the nuclease comprises a Class 2 nuclease. In embodiment 4 provided herein is the composition of embodiment 1, wherein the nuclease comprises a Type II or a Type V nuclease. In embodiment 5 provided herein is the composition of embodiment 1, wherein the nuclease comprises a Type V-A, V-B, V-C, V-D, or V-E nuclease. In embodiment 6 provided herein is the composition of embodiment 1, wherein the nuclease comprises a MAD nuclease, an ART nuclease, or an ABW nuclease. In embodiment 7 provided herein is the composition of embodiment 1, wherein the nuclease comprises a MAD1, MAD2, MAD3, MAD4, MAD5, MAD6, MAD7, MAD8, MAD9, MAD10, MAD11, MAD12, MAD13, MAD14, MAD15, MAD16, MAD17, MAD18, MAD19, or MAD20 nuclease. In embodiment 8 provided herein is the composition of embodiment 1, wherein the nuclease comprises an ART1, ART2, ART3, ART4, ART5, ART6, ART7, ART8, ART9, ART10, ART11, ART11*, ART12, ART13, ART14, ART15, ART16, ART17, ART18, ART19, ART20, ART21, ART22, ART23, ART24, ART25, ART26, ART27, ART28, ART29, ART30, ART31, ART32, ART33, ART34, or ART35 nuclease. In embodiment 9 provided herein is the composition of embodiment 1, wherein the nuclease comprises an amino acid sequence at least 80% identical to the amino acid sequence of MAD2, MAD7, ART2, ART11, or ART11* In embodiment 10 provided herein is the composition of embodiment 1, wherein the nuclease comprises at least one nuclear localization signal (NLS), at least one purification tag, or at least one cleavage site. In embodiment 11 provided herein is the composition of embodiment 10, wherein the nuclease comprises at least 4 NLS. In embodiment 12 provided herein is the composition of embodiment 1, wherein the gNA comprises a single polynucleotide. In embodiment 13 provided herein is the composition of embodiment 1, wherein the targeter nucleic acid and the modulator nucleic acid are separate polynucleotides, i.e., a dual gNA, wherein the dual gNA is capable of binding to and activating a nucleic acid-guided nucleases, that, in a naturally occurring system, is activated by a single crRNA in the absence of a tracrRNA. In embodiment 14 provided herein is the composition of embodiment 1, wherein the target nucleotide sequence is within at least 10, at least 20, at least 30, at least 40, or at least 50 nucleotides of a protospacer adjacent motif (PAM) that is recognized by a nuclease with which the guide nucleic acid is compatible. In embodiment 15 provided herein is the composition of embodiment 1, wherein the gNA and the nuclease form a nucleic acid-guided nuclease complex. In embodiment 16 provided herein is the composition of embodiment 15, wherein when the nucleic acid-guided nuclease complex is contacted with a genome of the human target cell, the complex hydrolyzes at least one strand in the target polynucleotide within or adjacent to the target nucleotide sequence. In embodiment 17 provided herein is the composition of embodiment 1, wherein the gNA comprises a spacer sequence comprising any one of SEQ ID NOs: 86-384. In embodiment 18 provided herein is the composition of embodiment 1, wherein some or all of the gNA is RNA. In embodiment 19 provided herein is the composition of embodiment 18, wherein at least 50%, at least 70%, at least 90%, at least 95%, or 100% of the gNA comprises RNA. In embodiment 20 provided herein is the composition of embodiment 1, wherein the gNA comprises one or more chemical modifications. In embodiment 21 provided herein is the composition of embodiment 20, wherein the chemical modification comprises a 2′-O-alkyl, a 2′-O-methyl, a phosphorothioate, a phosphonoacetate, a thiophosphonoacetate, a 2′-O-methyl-3′-phosphorothioate, a 2′-O-methyl-3′-phosphonoacetate, a 2′-O-methyl-3′-thiophosphonoacetate, a 2′-deoxy-3′-phosphonoacetate, a 2′-deoxy-3′-thiophosphonoacetate, a suitable alternative, or a combination thereof. In embodiment 22 provided herein is the composition of embodiment 1, wherein the proportion of gNA to nuclease is at least 1, 1.05 1.1, 1.15, 1.2, 1.25, 1.3, 1.35, 1.4, 1.45, 1.5, 1.55, 1.6, 1.65, 1.7, 1.75, 1.8, 1.85, 1.9, or 1.95 and/or not more than 1.05 1.1, 1.15, 1.2, 1.25, 1.3, 1.35, 1.4, 1.45, 1.5, 1.55, 1.6, 1.65, 1.7, 1.75, 1.8, 1.85, 1.9, 1.95 or 2 parts for every part of nuclease, for example, 1-2 parts of gNA for every part of nuclease. In embodiment 23 provided herein is the composition of embodiment 22, wherein the gNA and nuclease are present at 150:100 or 75:50 pmol. In embodiment 24 provided herein is the composition of embodiment 1, wherein the human target cell comprises an immune cell or a stem cell. In embodiment 25 provided herein is the composition of embodiment 24, wherein the immune cell is a neutrophil, eosinophil, basophil, mast cell, monocyte, macrophage, dendritic cell, natural killer cell, or a lymphocyte. In embodiment 26 provided herein is the composition of embodiment 24, wherein the immune cell comprises a T cell. In embodiment 27 provided herein is the composition of embodiment 26, wherein the immune cell comprises a CAR-T cell. In embodiment 28 provided herein is the composition of embodiment 24, wherein the stem cell comprises a human pluripotent, multipotent stem cell, embryonic stem cell, induced pluripotent stem cell, or hematopoietic stem cell. In embodiment 29 provided herein is the composition of embodiment 24, wherein the stem cell is a CD34+ stem cell. In embodiment 30 provided herein is the composition of embodiment 24, wherein the cell is an allogeneic cell. In embodiment 31 provided herein is the composition of embodiment 1, wherein the additive comprises an anionic polymer. In embodiment 32 provided herein is the composition of embodiment 1, wherein the additive comprises 1,2,3-heptanetriol, 2-Amino-2-(hydroxymethyl)-1,3-propanediol (Tris), 3-(1-pyridino)-1-propane sulfonate (NDSB 201), 3-[(3-cholamidopropyl)dimethylammonio]-1-propanesulfonate (CHAPS), 6-aminocaproic acid, adenosine diphosphate (ADP), adenosine triphosphate (ATP), alpha-cyclodextrin, amidosulfobetaine-14 (ASB-14), ammonium acetate, ammonium nitrate, ammonium sulfate, arginine, arginine ethylester, barium chloride, barium iodide, benzamidine HCl, beta-cyclodextrin, beta-mercaptoethanol (BME), biotin, calcium chloride, cesium chloride, cesium sulfate, cetyltrimethylammonium bromide (CTAB), choline chloride, citric acid, cobalt chloride, copper (II) chloride, cyclohexanol, D-sorbitol, dimethylethylammoniumpropane sulfonate (NDSB 195), dithiothreitol (DTT), erythritol, ethanol, ethylene glycol, ethylene glycol-bis(βbeta-aminoethyl ether)-N,N,N′,N′-tetraacetic acid (EGTA), ethylenediaminetetraacetic acid (EDTA), formamide, gadolinium bromide, gamma butyrolactone, glucose, glutamic acid, glutamine, glycerol, glycine, glycine betaine, glycine-glycine-glycine, guanidine HCl, guanosine triphosphate (GTP), holmium chloride, imidazole, iron (III) chloride, Jeffamine M-600, lanthanum acetate, lauryl sulfobetaine, lauryldimethylamine N-oxide (LDAO), lithium sulfate, magnesium chloride, magnesium sulfate, manganese chloride, mannitol, N-(2-hydroxyethyl) piperazine-N′-(3-propanesulfonic acid) (EPPS), N-dodecyl beta-D-maltoside (DDM), N-ethylurea, n-hexanol, N-lauryl sarcoside, N-lauryl sarcosine, N-methylformamide, N-methylurea, n-octyl-b-D-glucoside (OG: Octyl glucoside), n-penthanol, nickel chloride, non-detergent sulfo betaine (NDSB), Nonidet P40 (NP40), octyl beta-D-glucopyranoside, poly-L-glutamic acid, polyethylene glycol (for example, PEG 300, PEG 3350, PEG 4000), polyethyleneglycol lauryl ether (Brij 35), polyoxyethylene (2) oleyl ether (Brij 93), polyoxyethylene cetyl ether (Brij 56), polyvinylpyrrolidone 40 (PVP40), potassium chloride, potassium citrate, potassium nitrate, proline, putrescine, spermidine, spermine, riboflavin, samarium bromide, sarcosine, sodium acetate, sodium chloride, sodium dodecyl sulfate (SDS), sodium fluoride, sodium iodide, sodium lauroyl sarcosinate (Sarkosyl), sodium malonate, sodium molybdate, sodium selenite, sodium sulfate, sodium thiocyanate, sucrose, taurine, trehalose, tricine, triethylamine, trimethylamine N-oxide (TMAO), tris(2-carboxyethyl)phosphine (TCEP), Triton X-100, Tween 20, Tween 60, Tween 80, urea, vitamin B12, xylitol, yttrium chloride, yttrium nitrate, zinc chloride, Zwittergent 3-08, Zwittergent 3-14, or a combination thereof. In embodiment 33 provided herein is the composition of embodiment 1, wherein the additive comprises poly-L-glutamic acid (PGA). In embodiment 34 provided herein is the composition of embodiment 33, wherein the PGA is present at a concentration of at least 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, or 4.5 and/or not more than 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, or 5 μg μL⁻¹ per pmol RNP complex, for example 0.01-5 μg μL⁻¹ per pmol RNP complex. In embodiment 35 provided herein is the composition of embodiment 1, further comprising a donor template, wherein at least a portion of the donor template is capable of being inserted into the target polynucleotide at or near the site of cleavage. In embodiment 36 provided herein is the composition of embodiment 35, wherein the at least portion of the donor template is inserted by homology directed repair (HDR). In embodiment 37 provided herein is the composition of embodiment 35, wherein the donor template is single-stranded DNA, linear single-stranded RNA, linear double-stranded DNA, linear double-stranded RNA, circular single-stranded DNA, circular single-stranded RNA, circular double-stranded DNA, or circular double-stranded RNA. In embodiment 38 provided herein is the composition of embodiment 35, wherein the donor template comprises a mutation in a PAM sequence to partially or completely abolish binding of the RNP to the DNA. In embodiment 39 provided herein is the composition of embodiment 35, wherein the donor template comprises two homology arms. In embodiment 40 provided herein is the composition of embodiment 39, wherein the homology arms comprise at most 500 nucleotides. In embodiment 41 provided herein is the composition of embodiment 35, wherein the donor template comprises one or more promoters. In embodiment 42 provided herein is the composition of embodiment 41, wherein the promoter shares at least 70%, at least 75%, at least 80%, at least 85%, at least 90%, at least 95%, at least 99.5%, or 100%. sequence identity with any one of SEQ ID NOs: 78-85. In embodiment 43 provided herein is the composition of embodiment 1, wherein the RNP comprises a donor recruiting motif. In embodiment 44 provided herein is the composition of embodiment 35, wherein the donor template comprises a transgene. In embodiment 45 provided herein is the composition of embodiment 44, wherein the transgene comprises a fluorescent protein, a bioluminescent protein, an apoptotic switch, a cytokine, an interleukin, a gene circuit, a fusion protein, a CAAR, or a CAR component. In embodiment 46 provided herein is the composition of embodiment 45, wherein the CAR component is a B7H3, BCMA, GPRC5D, CD8, CD8a, CD19, CD20, CD22, CD28, 4-1BB, CD3zeta, or an engineered version thereof. In embodiment 47 provided herein is the composition of embodiment 1, the donor template is present at a concentration of at least 0.05, 0.01, 0.2, 0.3, 0.4, 0.5, 0.6, 0.7, 0.8, 0.9, 1, 1.25, 1.5, 1.75, 2, 3, or 4, and/or no more than 0.01, 0.2, 0.3, 0.4, 0.5, 0.6, 0.7, 0.8, 0.9, 1, 1.25, 1.5, 1.75, 2, 3, 4, or 5 μg μL⁻¹, for example 0.01-5 μg μL⁻¹. In embodiment 48 provided herein is the composition of embodiment 1, further comprising at least one additive that reduces non-homologous end joining (NHEJ)-based DNA repair. In embodiment 49 provided herein is the composition of embodiment 48, wherein the additive that reduces NHEJ is present in the recovery medium to which cells are added after delivery of the nuclease system and/or donor template. In embodiment 50 provided herein is the composition of embodiment 48, wherein the additive that reduces NHEJ comprises M3814. In embodiment 51 provided herein is the composition of embodiment 50, wherein the M3814 concentration is at least 0.1, 0.25, 0.5, 0.75, 1, 1.25, 1.5, 1.75, 2, 2.5, 3, or 4 and/or not more than 0.25, 0.5, 0.75, 1, 1.25, 1.5, 1.75, 2, 2.5, 3, 4, or 5 μM, for example 0.1-5 μM.

In embodiment 52 provided herein is a composition comprising: (a) a nucleic acid-guided nuclease capable of binding to a compatible guide nucleic acid (gNA) comprising a spacer sequence complementary to a target nucleotide sequence within a target polynucleotide, for example a target polynucleotide of a genome of a human target cell and generating a strand break in one or both strands of the target polynucleotide; (b) one or more human target cells; and c. at least one additive that reduces non-homologous end joining (NHEJ)-based DNA repair. In embodiment 53 provided herein is the composition of embodiment 52, wherein the nuclease comprises a Class 1 nuclease. In embodiment 54 provided herein is the composition of embodiment 52, wherein the nuclease comprises a Class 2 nuclease. In embodiment 55 provided herein is the composition of embodiment 52, wherein the nuclease comprises a Type II or a Type V nuclease. In embodiment 56 provided herein is the composition of embodiment 52, wherein the nuclease comprises a Type V-A, V-B, V-C, V-D, or V-E nuclease. In embodiment 57 provided herein is the composition of embodiment 52, wherein the nuclease comprises a MAD nuclease, an ART nuclease, or an ABW nuclease. In embodiment 58 provided herein is the composition of embodiment 52, wherein the nuclease comprises a MAD1, MAD2, MAD3, MAD4, MAD5, MAD6, MAD7, MAD8, MAD9, MAD10, MAD11, MAD12, MAD13, MAD14, MAD15, MAD16, MAD17, MAD18, MAD19, or MAD20 nuclease. In embodiment 59 provided herein is the composition of embodiment 52, wherein the nuclease comprises an ART1, ART2, ART3, ART4, ART5, ART6, ART7, ART8, ART9, ART10, ART11, ART11*, ART12, ART13, ART14, ART15, ART16, ART17, ART18, ART19, ART20, ART21, ART22, ART23, ART24, ART25, ART26, ART27, ART28, ART29, ART30, ART31, ART32, ART33, ART34, or ART35 nuclease. In embodiment 60 provided herein is the composition of embodiment 52, wherein the nuclease comprises an amino acid sequence at least 80% identical to the amino acid sequence of MAD2, MAD7, ART2, ART11, or ART11*. In embodiment 61 provided herein is the composition of embodiment 52, wherein the nuclease comprises at least one nuclear localization signal (NLS), at least one purification tag, or at least one cleavage site. In embodiment 62 provided herein is the composition of embodiment 61, wherein the nuclease comprises at least 4 NLS. In embodiment 63 provided herein is the composition of embodiment 52, further comprising a gNA, wherein the gNA is compatible with and capable of binding to and activating the nucleic acid-guided nuclease, wherein the gNA comprises: (a) a targeter nucleic acid comprising a targeter stem sequence and a spacer sequence, wherein the spacer sequence is complementary to a target nucleotide sequence within a target polynucleotide, for example a target polynucleotide of a genome of a human target cell; and (b) a modulator nucleic acid comprising a modulator stem sequence complementary to the targeter stem sequence, and, optionally, a 5′ sequence. In embodiment 64 provided herein is the composition of embodiment 63, wherein the gNA comprises a single polynucleotide. In embodiment 65 provided herein is the composition of embodiment 63, wherein the targeter nucleic acid and the modulator nucleic acid are separate polynucleotides, i.e., a dual gNA, wherein the dual gNA is capable of binding to and activating a nucleic acid-guided nucleases, that, in a naturally occurring system, is activated by a single crRNA in the absence of a tracrRNA. In embodiment 66 provided herein is the composition of embodiment 63, wherein the target nucleotide sequence is within at least 10, at least 20, at least 30, at least 40, or at least 50 nucleotides of a protospacer adjacent motif (PAM) that is recognized by a nuclease with which the guide nucleic acid is compatible. In embodiment 67 provided herein is the composition of embodiment 63, wherein the gNA and the nuclease form a nucleic acid-guided nuclease complex. In embodiment 68 provided herein is the composition of embodiment 67, wherein when the nucleic acid-guided nuclease complex is contacted with a genome of the human target cell, the complex hydrolyzes at least one strand in the target polynucleotide within or adjacent to the target nucleotide sequence. In embodiment 69 provided herein is the composition of embodiment 63, wherein the gNA comprises a spacer sequence comprising any one of SEQ ID NOs: 86-384. In embodiment 70 provided herein is the composition of embodiment 63, wherein some or all of the gNA is RNA. In embodiment 71 provided herein is the composition of embodiment 70, wherein at least 50%, at least 70%, at least 90%, at least 95%, or 100% of the gNA comprises RNA. In embodiment 72 provided herein is the composition of embodiment 63, wherein the gNA comprises one or more chemical modifications. In embodiment 73 provided herein is the composition of embodiment 72, wherein the chemical modification comprises a 2′-O-alkyl, a 2′-O-methyl, a phosphorothioate, a phosphonoacetate, a thiophosphonoacetate, a 2′-O-methyl-3′-phosphorothioate, a 2′-O-methyl-3′-phosphonoacetate, a 2′-O-methyl-3′-thiophosphonoacetate, a 2′-deoxy-3′-phosphonoacetate, a 2′-deoxy-3′-thiophosphonoacetate, a suitable alternative, or a combination thereof. In embodiment 74 provided herein is the composition of embodiment 63, wherein the proportion of gNA to nuclease is at least 1, 1.05 1.1, 1.15, 1.2, 1.25, 1.3, 1.35, 1.4, 1.45, 1.5, 1.55, 1.6, 1.65, 1.7, 1.75, 1.8, 1.85, 1.9, or 1.95 and/or not more than 1.05 1.1, 1.15, 1.2, 1.25, 1.3, 1.35, 1.4, 1.45, 1.5, 1.55, 1.6, 1.65, 1.7, 1.75, 1.8, 1.85, 1.9, 1.95 or 2 parts for every part of nuclease, for example, 1-2 parts of gNA for every part of nuclease. In embodiment 75 provided herein is the composition of embodiment 74, wherein the gNA and nuclease are present at 150:100 or 75:50 pmol. In embodiment 76 provided herein is the composition of embodiment 52, wherein the one or more human target cells comprise an immune cell or a stem cell. In embodiment 77 provided herein is the composition of embodiment 76, wherein the immune cell is a neutrophil, eosinophil, basophil, mast cell, monocyte, macrophage, dendritic cell, natural killer cell, or a lymphocyte. In embodiment 78 provided herein is the composition of embodiment 76, wherein the immune cell comprises a T cell. In embodiment 79 provided herein is the composition of embodiment 76, wherein the immune cell comprises a CAR-T cell. In embodiment 80 provided herein is the composition of embodiment 76, wherein the stem cell comprises a human pluripotent, multipotent stem cell, embryonic stem cell, induced pluripotent stem cell, or hematopoietic stem cell. In embodiment 81 provided herein is the composition of embodiment 76, wherein the stem cell is a CD34+ stem cell. In embodiment 82 provided herein is the composition of embodiment 76, wherein the cell is an allogeneic cell. In embodiment 83 provided herein is the composition of embodiment 52, further comprising at least one additive that stabilizes the type V nucleic acid-guided nuclease system. In embodiment 84 provided herein is the composition of embodiment 83, wherein the additive comprises an anionic polymer. In embodiment 85 provided herein is the composition of embodiment 83, wherein the additive comprises 1,2,3-heptanetriol, 2-Amino-2-(hydroxymethyl)-1,3-propanediol (Tris), 3-(1-pyridino)-1-propane sulfonate (NDSB 201), 3-[(3-cholamidopropyl)dimethylammonio]-1-propanesulfonate (CHAPS), 6-aminocaproic acid, adenosine diphosphate (ADP), adenosine triphosphate (ATP), alpha-cyclodextrin, amidosulfobetaine-14 (ASB-14), ammonium acetate, ammonium nitrate, ammonium sulfate, arginine, arginine ethylester, barium chloride, barium iodide, benzamidine HCl, beta-cyclodextrin, beta-mercaptoethanol (BME), biotin, calcium chloride, cesium chloride, cesium sulfate, cetyltrimethylammonium bromide (CTAB), choline chloride, citric acid, cobalt chloride, copper (II) chloride, cyclohexanol, D-sorbitol, dimethylethylammoniumpropane sulfonate (NDSB 195), dithiothreitol (DTT), erythritol, ethanol, ethylene glycol, ethylene glycol-bis(βbeta-aminoethyl ether)-N,N,N′,N′-tetraacetic acid (EGTA), ethylenediaminetetraacetic acid (EDTA), formamide, gadolinium bromide, gamma butyrolactone, glucose, glutamic acid, glutamine, glycerol, glycine, glycine betaine, glycine-glycine-glycine, guanidine HCl, guanosine triphosphate (GTP), holmium chloride, imidazole, iron (III) chloride, Jeffamine M-600, lanthanum acetate, lauryl sulfobetaine, lauryldimethylamine N-oxide (LDAO), lithium sulfate, magnesium chloride, magnesium sulfate, manganese chloride, mannitol, N-(2-hydroxyethyl) piperazine-N′-(3-propanesulfonic acid) (EPPS), N-dodecyl beta-D-maltoside (DDM), N-ethylurea, n-hexanol, N-lauryl sarcoside, N-lauryl sarcosine, N-methylformamide, N-methylurea, n-octyl-b-D-glucoside (OG: Octyl glucoside), n-penthanol, nickel chloride, non-detergent sulfo betaine (NDSB), Nonidet P40 (NP40), octyl beta-D-glucopyranoside, poly-L-glutamic acid, polyethylene glycol (for example, PEG 300, PEG 3350, PEG 4000), polyethyleneglycol lauryl ether (Brij 35), polyoxyethylene (2) oleyl ether (Brij 93), polyoxyethylene cetyl ether (Brij 56), polyvinylpyrrolidone 40 (PVP40), potassium chloride, potassium citrate, potassium nitrate, proline, putrescine, spermidine, spermine, riboflavin, samarium bromide, sarcosine, sodium acetate, sodium chloride, sodium dodecyl sulfate (SDS), sodium fluoride, sodium iodide, sodium lauroyl sarcosinate (Sarkosyl), sodium malonate, sodium molybdate, sodium selenite, sodium sulfate, sodium thiocyanate, sucrose, taurine, trehalose, tricine, triethylamine, trimethylamine N-oxide (TMAO), tris(2-carboxyethyl)phosphine (TCEP), Triton X-100, Tween 20, Tween 60, Tween 80, urea, vitamin B12, xylitol, yttrium chloride, yttrium nitrate, zinc chloride, Zwittergent 3-08, Zwittergent 3-14, or a combination thereof. In embodiment 86 provided herein is the composition of embodiment 83, wherein the additive comprises poly-L-glutamic acid (PGA). In embodiment 87 provided herein is the composition of embodiment 86, wherein the PGA is present at a concentration of at least 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, or 4.5 and/or not more than 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, or 5 μg μL⁻¹ per pmol RNP complex, for example 0.01-5 μg μL⁻¹ per pmol RNP complex. In embodiment 88 provided herein is the composition of embodiment 52, further comprising a donor template, wherein at least a portion of the donor template is capable of being inserted into the target polynucleotide at the site of cleavage. In embodiment 89 provided herein is the composition of embodiment 88, wherein the at least portion of the donor template is inserted by homology directed repair (HDR). In embodiment 90 provided herein is the composition of embodiment 88, wherein the donor template is single-stranded DNA, linear single-stranded RNA, linear double-stranded DNA, linear double-stranded RNA, circular single-stranded DNA, circular single-stranded RNA, circular double-stranded DNA, or circular double-stranded RNA. In embodiment 91 provided herein is the composition of embodiment 88, wherein the donor template comprises a mutation in a PAM sequence to partially or completely abolish binding of the RNP to the DNA. In embodiment 92 provided herein is the composition of embodiment 88, wherein the donor template comprises two homology arms. In embodiment 93 provided herein is the composition of embodiment 92, wherein the homology arms comprise at most 500 nucleotides. In embodiment 94 provided herein is the composition of embodiment 88, wherein the donor template comprises one or more promoters. In embodiment 95 provided herein is the composition of embodiment 94, wherein the promoter shares at least 70%, at least 75%, at least 80%, at least 85%, at least 90%, at least 95%, at least 99.5%, or 100%. sequence identity with any one of SEQ ID NOs: 78-85. In embodiment 96 provided herein is the composition of embodiment 52, wherein the RNP comprises a donor recruiting motif. In embodiment 97 provided herein is the composition of embodiment 88, wherein the donor template comprises a transgene. In embodiment 98 provided herein is the composition of embodiment 97, wherein the transgene comprises a fluorescent protein, a bioluminescent protein, an apoptotic switch, a cytokine, an interleukin, a gene circuit, a fusion protein, a CAAR, or a CAR component. In embodiment 99 provided herein is the composition of embodiment 98, wherein the CAR component is a B7H3, BCMA, GPRC5D, CD8, CD8a, CD19, CD20, CD22, CD28, 4-1BB, CD3zeta, or an engineered version thereof. In embodiment 100 provided herein is the composition of embodiment 52, wherein the donor template is present at a concentration of at least 0.05, 0.01, 0.2, 0.3, 0.4, 0.5, 0.6, 0.7, 0.8, 0.9, 1, 1.25, 1.5, 1.75, 2, 3, or 4, and/or no more than 0.01, 0.2, 0.3, 0.4, 0.5, 0.6, 0.7, 0.8, 0.9, 1, 1.25, 1.5, 1.75, 2, 3, 4, or 5 μg μL⁻¹, for example 0.01-5 μg μL⁻¹. In embodiment 101 provided herein is the composition of embodiment 89, wherein the at least one additive that reduces NHEJ results in an increased amount insertion of the at least portion of donor template via of HDR at or near the target site as compared NHEJ as measured by DNA sequencing. In embodiment 102 provided herein is the composition of embodiment 101, wherein the amount of HDR compared to NHEJ is increased by at least 1.2-fold, at least 1.4-fold, at least 1.6-fold, at least 1.8-fold, at least 2-fold, at least 2.5-fold, at least 3-fold, at least 3.5-fold, at least 4-fold, at least 4.5-fold, at least 5-fold, at least 6-fold, at least 7-fold, at least 8-fold, at least 9-fold, or at least 10-fold. In embodiment 103 provided herein is the composition embodiment 101, wherein the amount INDEL formation due to NHEJ as measured by sequencing is reduced by at least 1.2-fold, at least 1.4-fold, at least 1.6-fold, at least 1.8-fold, at least 2-fold, at least 2.5-fold, at least 3-fold, at least 3.5-fold, at least 4-fold, at least 4.5-fold, at least 5-fold, at least 6-fold, at least 7-fold, at least 8-fold, at least 9-fold, or at least 10-fold. In embodiment 104 provided herein is the composition of embodiment 52, wherein the additive that reduces NHEJ is present in the recovery medium to which cells are added after delivery of the nuclease system and/or donor template. In embodiment 105 provided herein is the composition of embodiment 52, wherein the additive that reduces NHEJ comprises M3814. In embodiment 106 provided herein is the composition of embodiment 104, wherein the M3814 concentration is at least 0.1, 0.25, 0.5, 0.75, 1, 1.25, 1.5, 1.75, 2, 2.5, 3, or 4 and/or not more than 0.25, 0.5, 0.75, 1, 1.25, 1.5, 1.75, 2, 2.5, 3, 4, or 5 μM, for example 0.1-5 μM.

In embodiment 107 provided herein is a composition comprising a human cell comprising: (a) a nuclease capable of binding to a compatible guide nucleic acid (gNA) comprising a spacer sequence complementary to a target nucleotide sequence within a target polynucleotide of a genome of the human cell and generating a strand break in one or both strands of the target polynucleotide; and (b) at least one additive that reduces non-homologous end joining (NHEJ)-based DNA repair. In embodiment 108 provided herein is the composition of embodiment 107, wherein the nuclease comprises a Class 1 nuclease. In embodiment 109 provided herein is the composition of embodiment 107, wherein the nuclease comprises a Class 2 nuclease. In embodiment 110 provided herein is the composition of embodiment 107, wherein the nuclease comprises a Type II or a Type V nuclease. In embodiment 111 provided herein is the composition of embodiment 107, wherein the nuclease comprises a Type V-A, V-B, V-C, V-D, or V-E nuclease. In embodiment 112 provided herein is the composition of embodiment 107, wherein the nuclease comprises a MAD nuclease, an ART nuclease, or an ABW nuclease. In embodiment 113 provided herein is the composition of embodiment 107, wherein the nuclease comprises a MAD1, MAD2, MAD3, MAD4, MAD5, MAD6, MAD7, MAD8, MAD9, MAD10, MAD11, MAD12, MAD13, MAD14, MAD15, MAD16, MAD17, MAD18, MAD19, or MAD20 nuclease. In embodiment 114 provided herein is the composition of embodiment 107, wherein the nuclease comprises an ART1, ART2, ART3, ART4, ART5, ART6, ART7, ART8, ART9, ART10, ART11, ART11*, ART12, ART13, ART14, ART15, ART16, ART17, ART18, ART19, ART20, ART21, ART22, ART23, ART24, ART25, ART26, ART27, ART28, ART29, ART30, ART31, ART32, ART33, ART34, or ART35 nuclease. In embodiment 115 provided herein is the composition of embodiment 107, wherein the nuclease comprises an amino acid sequence at least 80% identical to the amino acid sequence of MAD2, MAD7, ART2, ART11, or ART11*. In embodiment 116 provided herein is the composition of embodiment 107, wherein the nuclease comprises at least one nuclear localization signal (NLS), at least one purification tag, or at least one cleavage site. In embodiment 117 provided herein is the composition of embodiment 116, wherein the nuclease comprises at least 4 NLS. In embodiment 118 provided herein is the composition of embodiment 107, further comprising a gNA, wherein the gNA is compatible with and capable of binding to and activating the nucleic acid-guided nuclease, wherein the gNA comprises: (a) a targeter nucleic acid comprising a targeter stem sequence and a spacer sequence, wherein the spacer sequence is complementary to a target nucleotide sequence within a target polynucleotide, for example a target polynucleotide of a genome of a human target cell; and (b) a modulator nucleic acid comprising a modulator stem sequence complementary to the targeter stem sequence, and, optionally, a 5′ sequence. In embodiment 119 provided herein is the composition of embodiment 118, wherein the gNA comprises a single polynucleotide. In embodiment 120 provided herein is the composition of embodiment 118, wherein the targeter nucleic acid and the modulator nucleic acid are separate polynucleotides, i.e., a dual gNA, wherein the dual gNA is capable of binding to and activating a nucleic acid-guided nucleases, that, in a naturally occurring system, is activated by a single crRNA in the absence of a tracrRNA. In embodiment 121 provided herein is the composition of embodiment 118, wherein the target nucleotide sequence is within at least 10, at least 20, at least 30, at least 40, or at least 50 nucleotides of a protospacer adjacent motif (PAM) that is recognized by a nuclease with which the guide nucleic acid is compatible. In embodiment 122 provided herein is the composition of embodiment 118, wherein the gNA and the nuclease form a nucleic acid-guided nuclease complex. In embodiment 123 provided herein is the composition of embodiment 122, wherein when the nucleic acid-guided nuclease complex is contacted with a genome of the human target cell, the complex hydrolyzes at least one strand in the target polynucleotide within or adjacent to the target nucleotide sequence. In embodiment 124 provided herein is the composition of embodiment 118, wherein the gNA comprises a spacer sequence comprising any one of SEQ ID NOs: 86-384. In embodiment 125 provided herein is the composition of embodiment 118, wherein some or all of the gNA is RNA. In embodiment 126 provided herein is the composition of embodiment 125, wherein at least 50%, at least 70%, at least 90%, at least 95%, or 100% of the gNA comprises RNA. In embodiment 127 provided herein is the composition of embodiment 118, wherein the gNA comprises one or more chemical modifications. In embodiment 128 provided herein is the composition of embodiment 127, wherein the chemical modification comprises a 2′-O-alkyl, a 2′-O-methyl, a phosphorothioate, a phosphonoacetate, a thiophosphonoacetate, a 2′-O-methyl-3′-phosphorothioate, a 2′-O-methyl-3′-phosphonoacetate, a 2′-O-methyl-3′-thiophosphonoacetate, a 2′-deoxy-3′-phosphonoacetate, a 2′-deoxy-3′-thiophosphonoacetate, a suitable alternative, or a combination thereof. In embodiment 129 provided herein is the composition of embodiment 118, wherein the proportion of gNA to nuclease is at least 1, 1.05 1.1, 1.15, 1.2, 1.25, 1.3, 1.35, 1.4, 1.45, 1.5, 1.55, 1.6, 1.65, 1.7, 1.75, 1.8, 1.85, 1.9, or 1.95 and/or not more than 1.05 1.1, 1.15, 1.2, 1.25, 1.3, 1.35, 1.4, 1.45, 1.5, 1.55, 1.6, 1.65, 1.7, 1.75, 1.8, 1.85, 1.9, 1.95 or 2 parts for every part of nuclease, for example, 1-2 parts of gNA for every part of nuclease. In embodiment 130 provided herein is the composition of embodiment 129, wherein the gNA and nuclease are present at 150:100 or 75:50 pmol. In embodiment 131 provided herein is the composition of embodiment 107, wherein the one or more human target cells comprise an immune cell or a stem cell. In embodiment 132 provided herein is the composition of embodiment 131, wherein the immune cell is a neutrophil, eosinophil, basophil, mast cell, monocyte, macrophage, dendritic cell, natural killer cell, or a lymphocyte. In embodiment 133 provided herein is the composition of embodiment 131, wherein the immune cell comprises a T cell. In embodiment 134 provided herein is the composition of embodiment 131, wherein the immune cell comprises a CAR-T cell. In embodiment 135 provided herein is the composition of embodiment 131, wherein the stem cell comprises a human pluripotent, multipotent stem cell, embryonic stem cell, induced pluripotent stem cell, or hematopoietic stem cell. In embodiment 136 provided herein is the composition of embodiment 131, wherein the stem cell is a CD34+ stem cell. In embodiment 137 provided herein is the composition of embodiment 131, wherein the cell is an allogeneic cell. In embodiment 138 provided herein is the composition of embodiment 107, further comprising at least one additive that stabilizes the type V nucleic acid-guided nuclease system. In embodiment 139 provided herein is the composition of embodiment 138, wherein the additive comprises an anionic polymer. In embodiment 140 provided herein is the composition of embodiment 138, wherein the additive comprises 1,2,3-heptanetriol, 2-Amino-2-(hydroxymethyl)-1,3-propanediol (Tris), 3-(1-pyridino)-1-propane sulfonate (NDSB 201), 3-[(3-cholamidopropyl)dimethylammonio]-1-propanesulfonate (CHAPS), 6-aminocaproic acid, adenosine diphosphate (ADP), adenosine triphosphate (ATP), alpha-cyclodextrin, amidosulfobetaine-14 (ASB-14), ammonium acetate, ammonium nitrate, ammonium sulfate, arginine, arginine ethylester, barium chloride, barium iodide, benzamidine HCl, beta-cyclodextrin, beta-mercaptoethanol (BME), biotin, calcium chloride, cesium chloride, cesium sulfate, cetyltrimethylammonium bromide (CTAB), choline chloride, citric acid, cobalt chloride, copper (II) chloride, cyclohexanol, D-sorbitol, dimethylethylammoniumpropane sulfonate (NDSB 195), dithiothreitol (DTT), erythritol, ethanol, ethylene glycol, ethylene glycol-bis(βbeta-aminoethyl ether)-N,N,N′,N′-tetraacetic acid (EGTA), ethylenediaminetetraacetic acid (EDTA), formamide, gadolinium bromide, gamma butyrolactone, glucose, glutamic acid, glutamine, glycerol, glycine, glycine betaine, glycine-glycine-glycine, guanidine HCl, guanosine triphosphate (GTP), holmium chloride, imidazole, iron (III) chloride, Jeffamine M-600, lanthanum acetate, lauryl sulfobetaine, lauryldimethylamine N-oxide (LDAO), lithium sulfate, magnesium chloride, magnesium sulfate, manganese chloride, mannitol, N-(2-hydroxyethyl) piperazine-N′-(3-propanesulfonic acid) (EPPS), N-dodecyl beta-D-maltoside (DDM), N-ethylurea, n-hexanol, N-lauryl sarcoside, N-lauryl sarcosine, N-methylformamide, N-methylurea, n-octyl-b-D-glucoside (OG: Octyl glucoside), n-penthanol, nickel chloride, non-detergent sulfo betaine (NDSB), Nonidet P40 (NP40), octyl beta-D-glucopyranoside, poly-L-glutamic acid, polyethylene glycol (for example, PEG 300, PEG 3350, PEG 4000), polyethyleneglycol lauryl ether (Brij 35), polyoxyethylene (2) oleyl ether (Brij 93), polyoxyethylene cetyl ether (Brij 56), polyvinylpyrrolidone 40 (PVP40), potassium chloride, potassium citrate, potassium nitrate, proline, putrescine, spermidine, spermine, riboflavin, samarium bromide, sarcosine, sodium acetate, sodium chloride, sodium dodecyl sulfate (SDS), sodium fluoride, sodium iodide, sodium lauroyl sarcosinate (Sarkosyl), sodium malonate, sodium molybdate, sodium selenite, sodium sulfate, sodium thiocyanate, sucrose, taurine, trehalose, tricine, triethylamine, trimethylamine N-oxide (TMAO), tris(2-carboxyethyl)phosphine (TCEP), Triton X-100, Tween 20, Tween 60, Tween 80, urea, vitamin B12, xylitol, yttrium chloride, yttrium nitrate, zinc chloride, Zwittergent 3-08, Zwittergent 3-14, or a combination thereof. In embodiment 141 provided herein is the composition of embodiment 138, wherein the additive comprises poly-L-glutamic acid (PGA). In embodiment 142 provided herein is the composition of embodiment 141, wherein the PGA is present at a concentration of at least 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, or 4.5 and/or not more than 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, or 5 μg μL⁻¹ per pmol RNP complex, for example 0.01-5 μg μL⁻¹ per pmol RNP complex. In embodiment 143 provided herein is the composition of embodiment 107, further comprising a donor template, wherein at least a portion of the donor template is capable of being inserted into the target polynucleotide at the site of cleavage. In embodiment 144 provided herein is the composition of embodiment 143, wherein the at least portion of the donor template is inserted by homology directed repair (HDR). In embodiment 145 provided herein is the composition of embodiment 143, wherein the donor template is single-stranded DNA, linear single-stranded RNA, linear double-stranded DNA, linear double-stranded RNA, circular single-stranded DNA, circular single-stranded RNA, circular double-stranded DNA, or circular double-stranded RNA. In embodiment 146 provided herein is the composition of embodiment 143, wherein the donor template comprises a mutation in a PAM sequence to partially or completely abolish binding of the RNP to the DNA. In embodiment 147 provided herein is the composition of embodiment 143, wherein the donor template comprises two homology arms. In embodiment 148 provided herein is the composition of embodiment 147, wherein the homology arms comprise at most 500 nucleotides. In embodiment 149 provided herein is the composition of embodiment 143, wherein the donor template comprises one or more promoters. In embodiment 150 provided herein is the composition of embodiment 149, wherein the promoter shares at least 70%, at least 75%, at least 80%, at least 85%, at least 90%, at least 95%, at least 99.5%, or 100% sequence identity with any one of SEQ ID NOs: 78-85. In embodiment 151 provided herein is the composition of embodiment 107, wherein the RNP comprises a donor recruiting motif. In embodiment 152 provided herein is the composition of embodiment 143, wherein the donor template comprises a transgene. In embodiment 153 provided herein is the composition of embodiment 152, wherein the transgene comprises a fluorescent protein, a bioluminescent protein, an apoptotic switch, a cytokine, an interleukin, a gene circuit, a fusion protein, a CAAR, or a CAR component. In embodiment 154 provided herein is the composition of embodiment 153, wherein the CAR component is a B7H3, BCMA, GPRC5D, CD8, CD8a, CD19, CD20, CD22, CD28, 4-1BB, CD3zeta, or an engineered version thereof. In embodiment 155 provided herein is the composition of embodiment 107, wherein the donor template is present at a concentration of at least 0.05, 0.01, 0.2, 0.3, 0.4, 0.5, 0.6, 0.7, 0.8, 0.9, 1, 1.25, 1.5, 1.75, 2, 3, or 4, and/or no more than 0.01, 0.2, 0.3, 0.4, 0.5, 0.6, 0.7, 0.8, 0.9, 1, 1.25, 1.5, 1.75, 2, 3, 4, or 5 μg μL⁻¹, for example 0.01-5 μg μL⁻¹. In embodiment 156 provided herein is the composition of embodiment 144, wherein the at least one additive that reduces NHEJ results in an increased amount insertion of the at least portion of donor template via of HDR at or near the target site as compared NHEJ as measured by DNA sequencing. In embodiment 157 provided herein is the composition of embodiment 156, wherein the amount of HDR compared to NHEJ is increased by at least 1.2-fold, at least 1.4-fold, at least 1.6-fold, at least 1.8-fold, at least 2-fold, at least 2.5-fold, at least 3-fold, at least 3.5-fold, at least 4-fold, at least 4.5-fold, at least 5-fold, at least 6-fold, at least 7-fold, at least 8-fold, at least 9-fold, or at least 10-fold. In embodiment 158 provided herein is the composition embodiment 156, wherein the amount INDEL formation due to NHEJ as measured by sequencing is reduced by at least 1.2-fold, at least 1.4-fold, at least 1.6-fold, at least 1.8-fold, at least 2-fold, at least 2.5-fold, at least 3-fold, at least 3.5-fold, at least 4-fold, at least 4.5-fold, at least 5-fold, at least 6-fold, at least 7-fold, at least 8-fold, at least 9-fold, or at least 10-fold. In embodiment 159 provided herein is the composition of embodiment 107, wherein the additive that reduces NHEJ is present in the recovery medium to which cells are added after delivery of the nuclease system and/or donor template. In embodiment 160 provided herein is the composition of embodiment 107, wherein the additive that reduces NHEJ comprises M3814. In embodiment 161 provided herein is the composition of embodiment 159, wherein the M3814 concentration is at least 0.1, 0.25, 0.5, 0.75, 1, 1.25, 1.5, 1.75, 2, 2.5, 3, or 4 and/or not more than 0.25, 0.5, 0.75, 1, 1.25, 1.5, 1.75, 2, 2.5, 3, 4, or 5 M, for example 0.1-5 μM.

In embodiment 162 provided herein is a method for editing a target polynucleotide in the genome of a human target cell comprising: (a) contacting the target polynucleotide with a nuclease system comprising: (i) a nucleic acid-guided nuclease; and (ii) a guide nucleic acid (gNA) compatible with and capable of binding to and activating the nucleic acid-guided nuclease, wherein the gNA comprises: (1) a targeter nucleic acid comprising a targeter stem sequence and a spacer sequence, wherein the spacer sequence is complementary to a target nucleotide sequence within a target polynucleotide, for example a target polynucleotide of a genome of a human target cell; and (2) a modulator nucleic acid comprising a modulator stem sequence complementary to the targeter stem sequence, and, optionally, a 5′ sequence; and (b) contacting the cell with at least one additive that reduces non-homologous end joining (NHEJ)-based DNA repair. In embodiment 163 provided herein is the method of embodiment 162, further comprising, before contacting the target polynucleotide with the nuclease system. In embodiment 164 provided herein is the method of embodiment 163, wherein the nuclease system is delivered into the human target cell as one or more polynucleotides coding for one or more components of the system. In embodiment 165 provided herein is the method of embodiment 163, wherein the nuclease system is delivered into the human target cell as a pre-formed complex. In embodiment 166 provided herein is the method of embodiment 163, wherein the nuclease system is delivered into the human target cell by electroporation, lipofection, or a viral method. In embodiment 167 provided herein is the method of embodiment 163, further comprising, before delivering, combing the nuclease system with at least one additive that stabilizes the nuclease system. In embodiment 168 provided herein is the method of embodiment 167, wherein the additive that stabilizes the nuclease system is combined with the gNA prior to introduction of the nuclease. In embodiment 169 provided herein is the method of embodiment 162, wherein the nuclease system further comprises a donor template, wherein at least a portion of the donor template is capable of being inserted into the target polynucleotide at the site of cleavage. In embodiment 170 provided herein is the method of embodiment 162, wherein the additive that reduces NHEJ is present in the recovery medium to which cells are added after delivery of the nuclease system and/or donor template. In embodiment 171 provided herein is the method of embodiment 162, wherein the nuclease comprises a Class 1 nuclease. In embodiment 172 provided herein is the method of embodiment 162, wherein the nuclease comprises a Class 2 nuclease. In embodiment 173 provided herein is the method of embodiment 162, wherein the nuclease comprises a Type II or a Type V nuclease. In embodiment 174 provided herein is the method of embodiment 162, wherein the nuclease comprises a Type V-A, V-B, V-C, V-D, or V-E nuclease. In embodiment 175 provided herein is the method of embodiment 162, wherein the nuclease comprises a MAD nuclease, an ART nuclease, or an ABW nuclease. In embodiment 176 provided herein is the method of embodiment 162, wherein the nuclease comprises a MAD1, MAD2, MAD3, MAD4, MAD5, MAD6, MAD7, MAD8, MAD9, MAD10, MAD11, MAD12, MAD13, MAD14, MAD15, MAD16, MAD17, MAD18, MAD19, or MAD20 nuclease. In embodiment 177 provided herein is the method of embodiment 162, wherein the nuclease comprises an ART1, ART2, ART3, ART4, ART5, ART6, ART7, ART8, ART9, ART10, ART11, ART11*, ART12, ART13, ART14, ART15, ART16, ART17, ART18, ART19, ART20, ART21, ART22, ART23, ART24, ART25, ART26, ART27, ART28, ART29, ART30, ART31, ART32, ART33, ART34, or ART35 nuclease. In embodiment 178 provided herein is the method of embodiment 162, wherein the nuclease comprises an amino acid sequence at least 80% identical to the amino acid sequence of MAD2, MAD7, ART2, ART11, or ART11* In embodiment 179 provided herein is the method of embodiment 162, wherein the nuclease comprises at least one nuclear localization signal (NLS), at least one purification tag, or at least one cleavage site. In embodiment 180 provided herein is the method of embodiment 179, wherein the nuclease comprises at least 4 NLS. In embodiment 181 provided herein is the method of embodiment 162, wherein the gNA comprises a single polynucleotide. In embodiment 182 provided herein is the method of embodiment 162, wherein the targeter nucleic acid and the modulator nucleic acid are separate polynucleotides, i.e., a dual gNA, wherein the dual gNA is capable of binding to and activating a nucleic acid-guided nucleases, that, in a naturally occurring system, is activated by a single crRNA in the absence of a tracrRNA. In embodiment 183 provided herein is the method of embodiment 162, wherein the target nucleotide sequence is within at least 10, at least 20, at least 30, at least 40, or at least 50 nucleotides of a protospacer adjacent motif (PAM) that is recognized by a nuclease with which the guide nucleic acid is compatible. In embodiment 184 provided herein is the method of embodiment 162, wherein the gNA and the nuclease form a nucleic acid-guided nuclease complex. In embodiment 185 provided herein is the method of embodiment 184, wherein when the nucleic acid-guided nuclease complex is contacted with a genome of the human target cell, the complex hydrolyzes at least one strand in the target polynucleotide within or adjacent to the target nucleotide sequence. In embodiment 186 provided herein is the method of embodiment 162, wherein the gNA comprises a spacer sequence comprising any one of SEQ ID NOs: 86-384. In embodiment 187 provided herein is the method of embodiment 162, wherein some or all of the gNA is RNA. In embodiment 188 provided herein is the method of embodiment 187, wherein at least 50%, at least 70%, at least 90%, at least 95%, or 100% of the gNA comprises RNA. In embodiment 189 provided herein is the method of embodiment 162, wherein the gNA comprises one or more chemical modifications. In embodiment 190 provided herein is the method of embodiment 189, wherein the chemical modification comprises a 2′-O-alkyl, a 2′-O-methyl, a phosphorothioate, a phosphonoacetate, a thiophosphonoacetate, a 2′-O-methyl-3′-phosphorothioate, a 2′-O-methyl-3′-phosphonoacetate, a 2′-O-methyl-3′-thiophosphonoacetate, a 2′-deoxy-3′-phosphonoacetate, a 2′-deoxy-3′-thiophosphonoacetate, a suitable alternative, or a combination thereof. In embodiment 191 provided herein is the method of embodiment 162, wherein the proportion of gNA to nuclease is at least 1, at least 1.05, at least 1.1, at least 1.15, at least 1.2, at least 1.25, at least 1.3, at least 1.35, at least 1.4, at least 1.45, at least 1.5, at least 1.55, at least 1.6, at least 1.65, at least 1.7, at least 1.75, at least 1.8, at least 1.85, at least 1.9, at least 1.95, or at least 2 parts for every part of nuclease, for example, at least 1.5 parts of gNA for every part of nuclease. In embodiment 192 provided herein is the method of embodiment 191, wherein the gNA and nuclease are present at 150:100 or 75:50 pmol. In embodiment 193 provided herein is the method of embodiment 162, wherein the one or more human target cells comprise an immune cell or a stem cell. In embodiment 194 provided herein is the method of embodiment 193, wherein the immune cell comprises a neutrophil, eosinophil, basophil, mast cell, monocyte, macrophage, dendritic cell, natural killer cell, or a lymphocyte. In embodiment 195 provided herein is the method of embodiment 193, wherein the immune cell comprises a T cell. In embodiment 196 provided herein is the method of embodiment 193, wherein the immune cell comprises a CAR-T cell. In embodiment 197 provided herein is the method of embodiment 193, wherein the stem cell comprises a human pluripotent, multipotent stem cell, embryonic stem cell, induced pluripotent stem cell, or hematopoietic stem cell. In embodiment 198 provided herein is the method of embodiment 193, wherein the stem cell is a CD34+ stem cell. In embodiment 199 provided herein is the method of embodiment 193, wherein the cell is an allogeneic cell. In embodiment 200 provided herein is the method of embodiment 167, wherein the additive comprises an anionic polymer. In embodiment 201 provided herein is the method of embodiment 167, wherein the additive comprises 1,2,3-heptanetriol, 2-Amino-2-(hydroxymethyl)-1,3-propanediol (Tris), 3-(1-pyridino)-1-propane sulfonate (NDSB 201), 3-[(3-cholamidopropyl)dimethylammonio]-1-propanesulfonate (CHAPS), 6-aminocaproic acid, adenosine diphosphate (ADP), adenosine triphosphate (ATP), alpha-cyclodextrin, amidosulfobetaine-14 (ASB-14), ammonium acetate, ammonium nitrate, ammonium sulfate, arginine, arginine ethylester, barium chloride, barium iodide, benzamidine HCl, beta-cyclodextrin, beta-mercaptoethanol (BME), biotin, calcium chloride, cesium chloride, cesium sulfate, cetyltrimethylammonium bromide (CTAB), choline chloride, citric acid, cobalt chloride, copper (II) chloride, cyclohexanol, D-sorbitol, dimethylethylammoniumpropane sulfonate (NDSB 195), dithiothreitol (DTT), erythritol, ethanol, ethylene glycol, ethylene glycol-bis(βbeta-aminoethyl ether)-N,N,N′,N′-tetraacetic acid (EGTA), ethylenediaminetetraacetic acid (EDTA), formamide, gadolinium bromide, gamma butyrolactone, glucose, glutamic acid, glutamine, glycerol, glycine, glycine betaine, glycine-glycine-glycine, guanidine HCl, guanosine triphosphate (GTP), holmium chloride, imidazole, iron (III) chloride, Jeffamine M-600, lanthanum acetate, lauryl sulfobetaine, lauryldimethylamine N-oxide (LDAO), lithium sulfate, magnesium chloride, magnesium sulfate, manganese chloride, mannitol, N-(2-hydroxyethyl) piperazine-N′-(3-propanesulfonic acid) (EPPS), N-dodecyl beta-D-maltoside (DDM), N-ethylurea, n-hexanol, N-lauryl sarcoside, N-lauryl sarcosine, N-methylformamide, N-methylurea, n-octyl-b-D-glucoside (OG: Octyl glucoside), n-penthanol, nickel chloride, non-detergent sulfo betaine (NDSB), Nonidet P40 (NP40), octyl beta-D-glucopyranoside, poly-L-glutamic acid, polyethylene glycol (for example, PEG 300, PEG 3350, PEG 4000), polyethyleneglycol lauryl ether (Brij 35), polyoxyethylene (2) oleyl ether (Brij 93), polyoxyethylene cetyl ether (Brij 56), polyvinylpyrrolidone 40 (PVP40), potassium chloride, potassium citrate, potassium nitrate, proline, putrescine, spermidine, spermine, riboflavin, samarium bromide, sarcosine, sodium acetate, sodium chloride, sodium dodecyl sulfate (SDS), sodium fluoride, sodium iodide, sodium lauroyl sarcosinate (Sarkosyl), sodium malonate, sodium molybdate, sodium selenite, sodium sulfate, sodium thiocyanate, sucrose, taurine, trehalose, tricine, triethylamine, trimethylamine N-oxide (TMAO), tris(2-carboxyethyl)phosphine (TCEP), Triton X-100, Tween 20, Tween 60, Tween 80, urea, vitamin B12, xylitol, yttrium chloride, yttrium nitrate, zinc chloride, Zwittergent 3-08, Zwittergent 3-14, or a combination thereof. In embodiment 202 provided herein is the method of embodiment 167, wherein the additive comprises poly-L-glutamic acid (PGA). In embodiment 203 provided herein is the method of embodiment 202, wherein the PGA is present at a concentration of at least 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, or 4.5 and/or not more than 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, or 5 μg μL⁻¹ per pmol RNP complex, for example 0.01-5 μg μL⁻¹ per pmol RNP complex. In embodiment 204 provided herein is the method of embodiment 169, wherein the at least portion of the donor template is inserted by homology directed repair (HDR). In embodiment 205 provided herein is the method of embodiment 169, wherein the donor template is single-stranded DNA, linear single-stranded RNA, linear double-stranded DNA, linear double-stranded RNA, circular single-stranded DNA, circular single-stranded RNA, circular double-stranded DNA, or circular double-stranded RNA. In embodiment 206 provided herein is the method of embodiment 169, wherein the donor template comprises a mutation in a PAM sequence to partially or completely abolish binding of the RNP to the DNA. In embodiment 207 provided herein is the method of embodiment 169, wherein the donor template comprises two homology arms. In embodiment 208 provided herein is the method of embodiment 207, wherein the homology arms comprise at most 500 nucleotides. In embodiment 209 provided herein is the method of embodiment 169, wherein the donor template comprises one or more promoters. In embodiment 210 provided herein is the method of embodiment 209, wherein the promoter shares at least 70%, at least 75%, at least 80%, at least 85%, at least 90%, at least 95%, at least 99.5%, or 100% sequence identity with any one of SEQ ID NOs: 78-85. In embodiment 211 provided herein is the method of embodiment 162, wherein the RNP comprises a donor recruiting motif. In embodiment 212 provided herein is the method of embodiment 169, wherein the donor template comprises a transgene. In embodiment 213 provided herein is the method of embodiment 212, wherein the transgene comprises a fluorescent protein, a bioluminescent protein, an apoptotic switch, a cytokine, an interleukin, a gene circuit, a fusion protein, a CAAR, or a CAR component. In embodiment 214 provided herein is the method of embodiment 213, wherein the CAR component is a B7H3, BCMA, GPRC5D, CD8, CD8a, CD19, CD20, CD22, CD28, 4-1BB, CD3zeta, or an engineered version thereof. In embodiment 215 provided herein is the method of embodiment 169, wherein the donor template is present at a concentration of at least 0.05, 0.01, 0.2, 0.3, 0.4, 0.5, 0.6, 0.7, 0.8, 0.9, 1, 1.25, 1.5, 1.75, 2, 3, or 4, and/or no more than 0.01, 0.2, 0.3, 0.4, 0.5, 0.6, 0.7, 0.8, 0.9, 1, 1.25, 1.5, 1.75, 2, 3, 4, or 5 μg μL⁻¹, for example 0.01-5 μg μL⁻¹. In embodiment 216 provided herein is the method of embodiment 204, wherein the at least one additive that reduces NHEJ results in an increased amount insertion of the at least portion of donor template via of HDR at or near the target site as compared NHEJ as measured by DNA sequencing. In embodiment 217 provided herein is the method of embodiment 216, wherein the amount of HDR compared to NHEJ is increased by at least 1.2-fold, at least 1.4-fold, at least 1.6-fold, at least 1.8-fold, at least 2-fold, at least 2.5-fold, at least 3-fold, at least 3.5-fold, at least 4-fold, at least 4.5-fold, at least 5-fold, at least 6-fold, at least 7-fold, at least 8-fold, at least 9-fold, or at least 10-fold. In embodiment 218 provided herein is the method embodiment 216, wherein the amount INDEL formation due to NHEJ as measured by sequencing is reduced by at least 1.2-fold, at least 1.4-fold, at least 1.6-fold, at least 1.8-fold, at least 2-fold, at least 2.5-fold, at least 3-fold, at least 3.5-fold, at least 4-fold, at least 4.5-fold, at least 5-fold, at least 6-fold, at least 7-fold, at least 8-fold, at least 9-fold, or at least 10-fold. In embodiment 219 provided herein is the method of embodiment 162, wherein the additive that reduces non-homologous end joining comprises M3814. In embodiment 220 provided herein is the method of embodiment 219, wherein the M3814 concentration is at least 0.1, 0.25, 0.5, 0.75, 1, 1.25, 1.5, 1.75, 2, 2.5, 3, or 4 and/or not more than 0.25, 0.5, 0.75, 1, 1.25, 1.5, 1.75, 2, 2.5, 3, 4, or 5 μM, for example 0.1-5 μM.

### VIII. EXAMPLES

### Example 1: Culture of Jurkat Human T-Cell Leukemia Cell Line and Primary Human T-Cells

Human Jurkat T-cell leukemia cells (Leibniz Institute DSMZ-German Collection of Microorganisms and Cell Cultures GmbH (ACC 282)) were propagated in RPMI 1640 medium (ThermoFisher Scientific) with 10% heat-inactivated fetal bovine serum (FBS) (ThermoFisher Scientific) supplemented with 1% penicillin-streptomycin antibiotic mix (ThermoFisher Scientific). Cells were cultured at 37° C. in 5% CO2 incubators and maintained at a density of 0.5 to 1.5×10⁶ cells mL⁻¹. 24 hours before transfection, cells were passaged at 0.1×10⁶ cell mL⁻¹. Cell culture media supernatant was periodically tested for mycoplasma contamination using the MycoAlert PLUS mycoplasma detection kit (Lonza).

### Example 2: Primary T-Cell Isolation and Culture

T-cells were isolated from human peripheral blood obtained from healthy adults by immune-magnetic negative selection using the EasySep Human T-cell Isolation Kit (STEMCELL Technologies). After isolation, T-cells were activated in 25 μL mL⁻¹ ImmunoCult Human CD3/CD28/CD2 T-Cell Activator (STEMCELL Technologies) in ImmunoCult-XF T-Cell Expansion Medium (STEMCELL Technologies) containing 12.5 ng mL⁻¹ Human Recombinant IL-2, 5 ng mL⁻¹ IL-7, and 5 ng mL⁻¹ IL-15 (STEMCELL Technologies) and seeded at 1.0×10⁶ cells mL⁻¹. Until transfection 48 hours later, the cells were cultured at 37° C. in 5% CO2 incubators.

### Example 3: RNP Formulation

Ribonucleoprotein complexes (RNPs) were generated by incubating respective guide nucleic acids (gNAs) with MAD7 in the molar ratio of 3:2 gNA:MAD7 for 15 minutes at room temperature immediately before transfection. For Jurkat experiments, the RNP complexes were generated by mixing the respective gNA (150 pmol), MAD7 (100 pmol), and nuclease-free water, unless otherwise stated. For T-cell experiments, 1.6 μL of an aqueous solution of 15-50 kDa poly-L-glutamic acid (PGA, 100 μg μL⁻¹, Alamanda Polymers) was added to gNAs, followed by the addition of MAD7 and nuclease-free water.

### Example 4: Generation of Donor Template Via PCR Amplification

Donor templates comprising site-specific homology arms, respective promoter, and respective gene (GFP or Hu19 scFv-CD8α-CD28-CD3ζ CAR) were amplified from corresponding pTwist Ampicillin high-copy plasmids (Twist Bioscience) using homology arms-specific PCR primers. Donor templates were amplified in a two-step PCR program: initial denaturation at 98° C. for 30 seconds, cycle denaturation at 98° C. for 10 seconds, extension at 72° C. for 30 seconds per kb amplicon for 40-cycles with a hold at 72° C. for 10 minutes. Each 50 μL PCR reaction contained 10 ng amplification template (plasmid DNA), 0.5 μM homology arm-specific forward and reverse primers, nuclease-free water (IDT), 3% DMSO, and 1× Phusion High-Fidelity PCR Master Mix with HF Buffer (ThermoFisher Scientific). PCR products were purified using NucleoSpin Gel and PCR Clean-up Kit (Macherey-Nagel) with two 20 μL elutions. Purified HDR templates were collected and quantified on NanoDrop One Microvolume UV-Vis Spectrophotometer (ThermoFisher Scientific). Templates were concentrated using Amicon Ultra 0.5 mL 30K Centrifugal Filters: 100 μg DNA per unit was transferred, filled with nuclease-free water to 500 μL, and centrifuged at 10,000 g for 10 minutes to reduce volume to 50 μL. DNA was washed twice with nuclease-free water and recovered into a fresh tube by inversion and centrifugation at 10,000 g for 15 seconds. HDR templates were collected, diluted, and concentrations quantified using Qubit dsDNA HS Assay Kit (ThermoFisher Scientific). HDR templates of 0.5 to 1 μg μL⁻¹ were used for cellular studies.

### Example 5: Jurkat Cell Transfection

Lonza 4D Nucleofector with Shuttle unit (V4SC-2960 Nucleocuvette Strips) was used for transfection, following the manufacturer's instructions. For transfection, cells were harvested by centrifugation (200 g, RT, 5 minutes) and re-suspended in 20 μL at 10×10⁶ cells mL⁻¹ in the SF Cell Line Nucleofector X Kit buffer (Lonza), unless stated otherwise. The cell suspension was mixed with the RNPs, immediately transferred to the nucleocuvette, and transfected. After transfection, the cells were immediately re-suspended in the pre-warmed cultivation medium and plated onto 96-well, flat-bottom, non-cell culture treated plates (Falcon), and cultured at 37° C. in 5% CO2 incubators and maintained at a density of 0.5 to 1.0×10⁶ cells mL⁻¹. After 48 hours, the cells were harvested for the viability assay and genomic DNA, as described below. For the Homology-Directed Repair Template insertion, the HDR template was added to the cells and the suspension transferred to the RNPs immediately before transfection. The transfection parameters, cell recovery step, and proliferation conditions as described in Example 1. The cells were harvested 48 hours post-transfection for the viability assessment, after 7 days for CAR insertion efficiency, or after 7 days, 14 days, and 21 days for GFP insertion efficiency.

### Example 6: Primary T-Cell Transfection

48 hours after isolation, the cells were harvested by centrifugation (300 g, RT, 5 minutes) and re-suspended in 20 μL at 50×10⁶ cells mL⁻¹ in the supplemented P3 Primary Cell Nucleofector Kit buffer (Lonza). The cells were mixed with HDR templates and the suspension transferred to the RNPs immediately before transfection (Nucleofection program EH-115). After transfection, 80 μL of pre-warmed cultivation medium without IL-2 was added to the electroporation cuvettes. When using M3814 (Selleckchem), 80 μL of pre-warmed cultivation medium containing 2 μM M3814 final concentration without IL-2 was added to the electroporation cuvettes. After 10 minutes of incubation at 37° C., T-cells were transferred onto 96-well, flat-bottom, non-cell culture treated plates (Falcon) containing pre-warmed cultivation medium pretreated with 2 μM M3814 final concentration and 12.5 ng mL⁻¹ IL-2. The cells were seeded at a density of 0.25×10⁶ cells mL⁻¹, or 1.3×10⁶ cells mL⁻¹ in the experiment with M3814, and kept at 37° C. in 5% CO2 incubators. The viability assay was carried out 24 hours post-transfection after which the cells were reseeded in the fresh cultivation medium containing IL-2. Insertion efficiency of CAR was measured after 7 days, and 11 days or 13 days post-transfection.

### Example 7: Flow Cytometry

Flow cytometric assessments were carried out on a CytoFLEX S instrument (Beckmen Coulter) using a 96-well plate format. Measurements of cell viability, PDCD1 expression, GFP expression, and CAR expression were performed on 10,000 or 20,000 single cell events in Jurkat or primary T-cells, respectively.

For the cell viability and GFP knock-in measurements, approximately 250,000 cells per sample were transferred onto 96-well V-bottom cell culture plates and assessed following a series of consecutive washing and staining steps. The first step included centrifuging the cells at 300 g for 5 minutes at room temperature, discarding the supernatant, and washing cells in 150 μL Dulbecco's PBS/2% FBS (STEMCELL Technologies) or Cell Staining Buffer (Biolegend), respectively, followed by the second centrifugation and removal of supernatant. The final step included viability staining of cells using 150 μL Dulbecco's PBS/2% FBS with 7-amino-actinomycin D (7-AAD, 1:1,000; ThermoFisher) or 50 μL Cell Staining Buffer with Zombie Violet Dye (1:200; Biolegend), respectively. The measurements of cell viability and GFP expression were collected simultaneously for 7-AAD (excitation: yellow-green laser; emission: 561 nm), Zombie Violet (excitation: violet laser; emission 405 nm), and GFP (excitation: blue laser; emission 488 nm) as needed.

For detection of CAR knock-in efficiency, approx. 250,000 cells per sample were transferred onto 96-well V-bottom, washed as described above using Cell Staining Buffer, and re-suspended in 50 μL Cell Staining Buffer with PE Anti-Myc tag antibody [9E10] (1:50; Abcam) and Zombie Violet Dye (1:200; Biolegend) for 30 minutes. Afterwards, the cells were washed in two subsequent washing steps using 150 μL Cell Staining Buffer, and finally re-suspended in 100 μL Cell Staining Buffer for the flow cytometry measurements (excitation: yellow-green laser; emission: 561 nm).

For detection of PDCD1 knock-out efficiency, approx. 250,000 Jurkat cells per sample were transferred onto 96-well V-bottom cell culture plates and assessed following a series of consecutive washing and staining steps. The first step included centrifuging the cells at 300 g for 5 minutes at 4° C. and discarding the supernatant. Afterwards, the cells were stained using 100 μL Cell Staining Buffer (Biolegend) with APC/Cyanine7 anti-human CD279 (PD-1) antibody (1:100; Biolegend) and incubated for 30 minutes at 4° C. in the dark. The cells were then centrifuged at 300 g for 5 minutes at 4° C. and the supernatant discarded. The next step included two repeats of centrifugation at 300 g for 5 minutes at 4° C., supernatant removal, and cell washing in 150 μL ice-cold Cell Staining Buffer (Biolegend). In the final step, the cells were re-suspended in 100 μL Cell Staining Buffer for the flow cytometry measurements (excitation: red laser; emission: 633 nm).

### Example 8: DNA Extraction

Cells were harvested 48-h post-transfection by centrifugation (1,000 g, 10 minutes) in 96-well, V-bottom plates (Greiner), washed with PBS (Sigma Aldrich) and lysed in 20 μL QuickExtract DNA Extraction Solution (Epicentre, Lucigen). DNA was extracted following the manufacturer's protocol: 15 minutes at 65° C., 15 minutes at 68° C., 10 minutes at 95° C., cooled to 4° C., and stored at 4° C. Genomic DNA was diluted 20-fold in nuclease-free water before amplicon PCR reactions.

### Example 9: Amplicon Sequencing

Extracted genomic DNA was quantified using the NanoDrop (ThermoFisher Scientific). Amplicons were constructed in two PCR steps: in the first PCR, regions of interest (150-400 bp) were amplified from 10 to 30 ng of genomic DNA with primers containing Illumina forward and reverse adapters on both ends comprising loci-specific complementary sequences as shown in Table 6, using Phusion High-Fidelity PCR Master Mix (ThermoFisher Scientific). Amplification products were purified with Agencourt AMPure XP beads (Ramcon), using the sample to beads ratio of 1:1.8. The DNA was eluted from the beads with nuclease-free water and the size of the purified amplicons analyzed on a 2% agarose E-gel using the E-gel electrophoresis system (ThermoFisher Scientific). In the second PCR, unique pairs of Illumina-compatible indexes (Nextera XT Index Kit v2) were added to the amplicons using the KAPA HiFi HotStart Ready Mix (Roche). The amplified products were purified with Agencourt AMPure XP beads (Ramcon), using the sample to bead ratio of 1:1.8. The DNA was eluted from the beads with 10 mM Tris-HCl pH 8.5, 0.1% Tween 20. Sizes of the purified DNA fragments were validated on a 2% agarose gel using the E-gel electrophoresis system (ThermoFisher Scientific), quantified using Qubit dsDNA HS Assay Kit (Thermo Fisher) and then pooled in equimolar concentrations. Quality of the amplicon library was validated using Bioanalyzer, High Sensitivity DNA Kit (Agilent) before sequencing. The final library was sequenced on Illumina MiSeq System using the MiSeq Reagent Kit v.2 (300 cycles, 2×250 bp, paired-end reads). De-multiplexed FASTQ files were obtained from BaseSpace (Illumina).

TABLE 6
Primer sequences

| | SEQ ID | | SEQ ID | |
|---|---|---|---|---|
| Name | NO | Forward primer | NO | Reverse primer |
| crCD247_ | 385 | TGGGGAGGTAGCTGCAGAA | 684 | CTAGAAGTTCCCTGCCG |
| 1 | | T | | TCG |
| crCD247_ | 386 | TGGGGAGGTAGCTGCAGAA | 685 | CTAGAAGTTCCCTGCCG |
| 2 | | T | | TCG |
| crCD247_ | 387 | TGGGGATGTGTTCTCGTCA | 686 | GCCCCTCTGAACATCCA |
| 3 | | C | | TCA |
| crCD247_ | 388 | GGTAGCACAGGGAGGAGAG | 687 | GCCCTTCCTCCAACTTT |
| 4 | | A | | CCA |
| crCD247_ | 389 | TTAGTTGCCAAGGAGCGGA | 688 | GGCGAGGCTGACTTACG |
| 5 | | G | | TTA |
| crCD247_ | 390 | GCCCTTCCTCCAACTTTCC | 689 | GGTAGCACAGGGAGGAG |
| 6 | | A | | AGA |
| crCD247_ | 391 | GGTAGCACAGGGAGGAGAG | 690 | GCCCTTCCTCCAACTTT |
| 7 | | A | | CCA |
| crCD247_ | 392 | CGTGTCTGGAGGACCAAGA | 691 | CTGGTTGTGGGCAGAGA |
| 8 | | G | | AGT |
| crCD247_ | 393 | CTGGTTGTGGGCAGAGAAG | 692 | CGTGTCTGGAGGACCAA |
| 9 | | T | | GAG |
| crCD247_ | 394 | TGCAGCTGGGATGAGAAGT | 693 | TGGAGCCTTGATTGTGG |
| 10 | | G | | GAG |
| crCD247_ | 395 | TGCAGCTGGGATGAGAAGT | 694 | TGGAGCCTTGATTGTGG |
| 11 | | G | | GAG |
| crCD247_ | 396 | GGCCTCACCTTACTCTGCA | 695 | ATCTTGCCCCTTGTCAG |
| 12 | | G | | GTG |
| crCD247_ | 397 | GGCCTCACCTTACTCTGCA | 696 | ATCTTGCCCCTTGTCAG |
| 13 | | G | | GTG |
| crCD247_ | 398 | GGCCTCACCTTACTCTGCA | 697 | ATCTTGCCCCTTGTCAG |
| 14 | | G | | GTG |
| crCD247_ | 399 | TAAACCCAAGACTCTGGCG | 698 | TTAGTTGCCAAGGAGCG |
| 15 | | G | | GAG |
| crCD247_ | 400 | ACAGCACCCATCTACCAAC | 699 | GTCTGGCCTTTGAGTGG |
| 16 | | G | | TGA |
| crCD247_ | 401 | ACAGCACCCATCTACCAAC | 700 | GTCTGGCCTTTGAGTGG |
| 17 | | G | | TGA |
| crCD247_ | 402 | ACAGCACCCATCTACCAAC | 701 | GTCTGGCCTTTGAGTGG |
| 18 | | G | | TGA |
| crCD247_ | 403 | CAGGGGGATTATTCCTGGG | 702 | ATAATCTGGGCGTCTGC |
| 19 | | C | | AGG |
| crCD247_ | 404 | TATGGCGCCCTTTGAGACA | 703 | TGTGTTGCAGTTCAGCA |
| 20 | | G | | GGA |
| crCD247_ | 405 | GCCCCTGCCCCTCTTTTTA | 704 | TGGTTGCAGAGTGAGCT |
| 21 | | T | | GAG |
| crCD247_ | 406 | GCCCCTGCCCCTCTTTTTA | 705 | TGGTTGCAGAGTGAGCT |
| 22 | | T | | GAG |
| crCD247_ | 407 | TGGTTGCAGAGTGAGCTGA | 706 | GCCCCTGCCCCTCTTTT |
| 23 | | G | | TAT |
| crCD247_ | 408 | TGGTTGCAGAGTGAGCTGA | 707 | GCCCCTGCCCCTCTTTT |
| 24 | | G | | TAT |
| crCD247_ | 409 | GCCCCTGCCCCTCTTTTTA | 708 | TGGTTGCAGAGTGAGCT |
| 25 | | T | | GAG |
| crCD247_ | 410 | GGTAGCACAGGGAGGAGAG | 709 | GCCCTTCCTCCAACTTT |
| 26 | | A | | CCA |
| crCTLA4_ | 411 | ATCATGTAGGTTGCCGCAC | 710 | GGCCATGAAGGAGCATG |
| 1 | | A | | AGT |
| crCTLA4_ | 412 | TCACTGCCTTTGACTGCTG | 711 | TGAAGACCTGAACACCG |
| 2 | | A | | CTC |
| crCTLA4_ | 413 | AAATCTGGGTTCCGTTGCC | 712 | AGGTGACTGAAGTCTGT |
| 3 | | T | | GCG |
| crCTLA4_ | 414 | GGCCATGAAGGAGCATGAG | 713 | ATCATGTAGGTTGCCGC |
| 4 | | T | | ACA |
| crCTLA4_ | 415 | AGTCCTTGATTCTGTGTGG | 714 | CCTCCTCCATCTTCATG |
| 5 | | GT | | CTCC |
| crCTLA4_ | 416 | CCTCCTCCATCTTCATGCT | 715 | AGTCCTTGATTCTGTGT |
| 6 | | CC | | GGGT |
| crCTLA4_ | 417 | AAGCTAGAAGGCAGAAGGG | 716 | ATCATGTAGGTTGCCGC |
| 7 | | C | | ACA |
| crCTLA4_ | 418 | AAGCTAGAAGGCAGAAGGG | 717 | ATCATGTAGGTTGCCGC |
| 8 | | C | | ACA |
| crCTLA4_ | 419 | GGCCATGAAGGAGCATGAG | 718 | ATCATGTAGGTTGCCGC |
| 9 | | T | | ACA |
| crCTLA4_ | 420 | GGCCATGAAGGAGCATGAG | 719 | ATCATGTAGGTTGCCGC |
| 10 | | T | | ACA |
| crCTLA4_ | 421 | CATGCTAGCAATGCACGTG | 720 | TGATTTCCACTGGAGGT |
| 11 | | G | | GCC |
| crCTLA4_ | 422 | CATGCTAGCAATGCACGTG | 721 | TGATTTCCACTGGAGGT |
| 12 | | G | | GCC |
| crCTLA4_ | 423 | CATCGCCAGCTTTGTGTGT | 722 | GAGCTCCACCTTGCAGA |
| 13 | | G | | TGT |
| crCTLA4_ | 424 | AGTCCTTGATTCTGTGTGG | 723 | CCTCCTCCATCTTCATG |
| 14 | | GT | | CTCC |
| crCTLA4_ | 425 | AGGTGACTGAAGTCTGTGC | 724 | AAATCTGGGTTCCGTTG |
| 15 | | G | | CCT |
| crCTLA4_ | 426 | AGGTGACTGAAGTCTGTGC | 725 | AAATCTGGGTTCCGTTG |
| 16 | | G | | CCT |
| crCTLA4_ | 427 | AGGTGACTGAAGTCTGTGC | 726 | AAATCTGGGTTCCGTTG |
| 17 | | G | | CCT |
| crCTLA4_ | 428 | CATCTGCAAGGTGGAGCTC | 727 | GGTTGCCACCCACAATA |
| 18 | | A | | AGC |
| crCTLA4_ | 429 | TCTGCAAGGTGGAGCTCAT | 728 | GGTTGCCACCCACAATA |
| 19 | | G | | AGC |
| crCTLA4_ | 430 | GCAATTTAGGGGTGGACCT | 729 | CATCAGCACCACACTCA |
| 20 | | CA | | CCA |
| crCTLA4_ | 431 | GCAATTTAGGGGTGGACCT | 730 | CATCAGCACCACACTCA |
| 21 | | CA | | CCA |
| crCTLA4_ | 432 | AATGTTGGGGAGTAGAGCC | 731 | ATCCCCATCAGACATGG |
| 22 | | C | | TGC |
| crCTLA4_ | 433 | CAATGTTGGGGAGTAGAGC | 732 | GCACCACACTCACCATT |
| 23 | | CCT | | TTGCT |
| crCTLA4_ | 434 | ATGTTGGGGAGTAGAGCCC | 733 | ATCCCCATCAGACATGG |
| 24 | | T | | TGC |
| crCTLA4_ | 435 | AGTCCTTGATTCTGTGTGG | 734 | CCTCCTCCATCTTCATG |
| 25 | | GT | | CTCC |
| crCTLA4_ | 436 | ATGTTGGGGAGTAGAGCCC | 735 | ATCCCCATCAGACATGG |
| 26 | | T | | TGC |
| crCTLA4_ | 437 | ATGTTGGGGAGTAGAGCCC | 736 | ATCCCCATCAGACATGG |
| 27 | | T | | TGC |
| crCTLA4_ | 438 | ATGTTGGGGAGTAGAGCCC | 737 | ATCCCCATCAGACATGG |
| 28 | | T | | TGC |
| crCTLA4_ | 439 | ATGTTGGGGAGTAGAGCCC | 738 | ATCCCCATCAGACATGG |
| 29 | | T | | TGC |
| crCTLA4_ | 440 | AGGGACCCAATATGTGTTG | 739 | TGCCTCAGCTCTTGGAA |
| 30 | | AGT | | ATTG |
| crCTLA4_ | 441 | AGGGACCCAATATGTGTTG | 740 | TGCCTCAGCTCTTGGAA |
| 31 | | AGT | | ATTG |
| crCTLA4_ | 442 | AGGGACCCAATATGTGTTG | 741 | TGCCTCAGCTCTTGGAA |
| 32 | | AGT | | ATTG |
| crCTLA4_ | 443 | TGGTTAGAAGTGGCTTCCG | 742 | AGAATTGCCTCAGCTCT |
| 33 | | T | | TGGA |
| crCTLA4_ | 444 | TGGTTAGAAGTGGCTTCCG | 743 | AGAATTGCCTCAGCTCT |
| 34 | | T | | TGGA |
| crCTLA4_ | 445 | TGGTTAGAAGTGGCTTCCG | 744 | AGAATTGCCTCAGCTCT |
| 35 | | T | | TGGA |
| crCTLA4_ | 446 | CCCTCTTACAACAGGGGTC | 745 | TGGGTTCCGCATCCAAC |
| 36 | | T | | TTT |
| crCTLA4_ | 447 | CCCTCTTACAACAGGGGTC | 746 | TGGGTTCCGCATCCAAC |
| 37 | | T | | TTT |
| crCTLA4_ | 448 | TGAAGACCTGAACACCGCT | 747 | TCACTGCCTTTGACTGC |
| 38 | | C | | TGA |
| crCTLA4_ | 449 | TGAAGACCTGAACACCGCT | 748 | TCACTGCCTTTGACTGC |
| 39 | | C | | TGA |
| crCTLA4_ | 450 | AAGCTAGAAGGCAGAAGGG | 749 | ATCATGTAGGTTGCCGC |
| 40 | | C | | ACA |
| crCTLA4_ | 451 | AAGCTAGAAGGCAGAAGGG | 750 | ATCATGTAGGTTGCCGC |
| 41 | | C | | ACA |
| crLAG3_ | 452 | TAGTGAAGCCTCTCCAGCC | 751 | AGGGAGTGACACCTCAG |
| 1 | | A | | GG |
| crLAG3_ | 453 | CCAAGTGAGTGCAGGGTGA | 752 | GTGTCCAGAGAGCTCCA |
| 2 | | T | | CAC |
| crLAG3_ | 454 | TGGGGAAGCTGCTTTGTGA | 753 | TTTGGGTCCTGGCATTC |
| 3 | | G | | TGG |
| crLAG3_ | 455 | CTGGATCCCTGGGGAAGCT | 754 | TGGCGTTTGGGTCCTGG |
| 4 | | GCT | | CATTC |
| crLAG3_ | 456 | CCAAGTGAGTGCAGGGTGA | 755 | CCAGCCAAGGTCCTGAG |
| 5 | | T | | AAA |
| crLAG3_ | 457 | CCTTTTGGAGGGCTCAGCG | 756 | CCAGAGAGGCTTTCGGG |
| 6 | | CTG | | GTGGA |
| crLAG3_ | 458 | CTGAGATGGGGAGAGGGTG | 757 | TTCCGGAACCAATGCAC |
| 7 | | A | | AGA |
| crLAG3_ | 459 | TCCAGTGGGCTGATGAAGT | 758 | CTTGGGGCAGGAAGAGG |
| 8 | | C | | AAG |
| crLAG3_ | 460 | TCCAGTGGGCTGATGAAGT | 759 | CTTGGGGCAGGAAGAGG |
| 9 | | C | | AAG |
| crLAG3_ | 461 | GGATCTCTCAGAGCCTCCG | 760 | CTGTAGGTGAGGATGCA |
| 10 | | A | | GCC |
| crLAG3_ | 462 | GGATCTCTCAGAGCCTCCG | 761 | CTGTAGGTGAGGATGCA |
| 11 | | A | | GCC |
| crLAG3_ | 463 | GCCCAGCCTCTGTGCATTG | 762 | GGGGGCAGGAAGGAGTT |
| 12 | | GTT | | GTGGT |
| crLAG3_ | 464 | GCCCAGCCTCTGTGCATTG | 763 | GGGGGCAGGAAGGAGTT |
| 13 | | GTT | | GTGGT |
| crLAG3_ | 465 | GCCCAGCCTCTGTGCATTG | 764 | GGGGGCAGGAAGGAGTT |
| 14 | | GTT | | GTGGT |
| crLAG3_ | 466 | CTTCCTCTTCCTGCCCCAA | 765 | ACCCACAGCAATGACGT |
| 15 | | G | | AGG |
| crLAG3_ | 467 | TGAGCCAGACCATCTCCTG | 766 | CAGTGAGGAAAGACCGG |
| 16 | | A | | GTC |
| crLAG3_ | 468 | CCTTTTGGAGGGCTCAGCG | 767 | CCAGAGAGGCTTTCGGG |
| 17 | | CTG | | GTGGA |
| crLAG3_ | 469 | TGAGCCAGACCATCTCCTG | 768 | CAGTGAGGAAAGACCGG |
| 18 | | A | | GTC |
| crLAG3_ | 470 | TGAGCCAGACCATCTCCTG | 769 | CAGTGAGGAAAGACCGG |
| 19 | | A | | GTC |
| crLAG3_ | 471 | GTCTGGAGCCCCCAACTCC | 770 | CTGGGCCTGGCTCACAT |
| 20 | | CTT | | CCTCT |
| crLAG3_ | 472 | GTCTGGAGCCCCCAACTCC | 771 | CTGGGCCTGGCTCACAT |
| 21 | | CTT | | CCTCT |
| crLAG3_ | 473 | GACCCGGTCTTTCCTCACT | 772 | GAGGGCAGCTACTCCTT |
| 22 | | G | | TCC |
| crLAG3_ | 474 | GACCCGGTCTTTCCTCACT | 773 | GAGGGCAGCTACTCCTT |
| 23 | | G | | TCC |
| crLAG3_ | 475 | GACCCGGTCTTTCCTCACT | 774 | GAGGGCAGCTACTCCTT |
| 24 | | G | | TCC |
| crLAG3_ | 476 | TGGCGACTTTACCCTTCGA | 775 | CTCTGGAACTTGTGCCC |
| 25 | | C | | AGT |
| crLAG3_ | 477 | TGGCGACTTTACCCTTCGA | 776 | CTCTGGAACTTGTGCCC |
| 26 | | C | | AGT |
| crLAG3_ | 478 | CCAAGTGAGTGCAGGGTGA | 777 | GTGTCCAGAGAGCTCCA |
| 27 | | T | | CAC |
| crLAG3_ | 479 | CCTTTTGGAGGGCTCAGCG | 778 | CCAGAGAGGCTTTCGGG |
| 28 | | CTG | | GTGGA |
| crLAG3_ | 480 | CCAAGTGAGTGCAGGGTGA | 779 | GTGTCCAGAGAGCTCCA |
| 29 | | T | | CAC |
| crLAG3_ | 481 | CCAAGTGAGTGCAGGGTGA | 780 | GTGTCCAGAGAGCTCCA |
| 30 | | T | | CAC |
| crLAG3_ | 482 | CCAAGTGAGTGCAGGGTGA | 781 | GTGTCCAGAGAGCTCCA |
| 31 | | T | | CAC |
| crLAG3_ | 483 | CCAAGTGAGTGCAGGGTGA | 782 | CCAGCCAAGGTCCTGAG |
| 32 | | T | | AAA |
| crLAG3_ | 484 | TCCTTTGGGTCACCTGGAT | 783 | CTGCTCCAAGAAGCCTC |
| 33 | | C | | TCC |
| crLAG3_ | 485 | TCCTTTGGGTCACCTGGAT | 784 | CTGCTCCAAGAAGCCTC |
| 34 | | C | | TCC |
| crLAG3_ | 486 | AGAACGCTTTGTGTGGAGC | 785 | TTTGGGTCCTGGCATTC |
| 35 | | T | | TGG |
| crLAG3_ | 487 | TTCCTGCACCCTGTTTCTC | 786 | GCAGAAGGCTGAGATCC |
| 36 | | C | | TGG |
| crLAG3_ | 488 | AGAACGCTTTGTGTGGAGC | 787 | TTTGGGTCCTGGCATTC |
| 37 | | T | | TGG |
| crLAG3_ | 489 | CTGGATCCCTGGGGAAGCT | 788 | TGGCGTTTGGGTCCTGG |
| 38 | | GCT | | CATTC |
| crLAG3_ | 490 | TTTCTCAGGACCTTGGCTG | 789 | AAGCCAGAGATCAGGTC |
| 39 | | G | | CCT |
| crLAG3_ | 491 | CTTTCCCAGCCTTGGCAAT | 790 | AAGCCAGAGATCAGGTC |
| 40 | | G | | CCT |
| crLAG3_ | 492 | GCTGAATGACCCTGGGACA | 791 | GGCTCCAGTCACCAAAA |
| 41 | | A | | GGA |
| crLAG3_ | 493 | GCTGAATGACCCTGGGACA | 792 | GGCTCCAGTCACCAAAA |
| 42 | | A | | GGA |
| crLAG3_ | 494 | CCATAGGTGCCCAACGCTC | 793 | TGAGGGCAAGTTCAGGG |
| 43 | | TGG | | TCCCA |
| crLAG3_ | 495 | CCATAGGTGCCCAACGCTC | 794 | TGAGGGCAAGTTCAGGG |
| 44 | | TGG | | TCCCA |
| crLAG3_ | 496 | CCATAGGTGCCCAACGCTC | 795 | TGAGGGCAAGTTCAGGG |
| 45 | | TGG | | TCCCA |
| crLAG3_ | 497 | GGCCTCTCTTTTGCTCACC | 796 | GGTTGAGTGCTGGATTC |
| 46 | | T | | GGA |
| crLAG3_ | 498 | CCATAGGTGCCCAACGCTC | 797 | TGAGGGCAAGTTCAGGG |
| 47 | | TGG | | TCCCA |
| crLAG3_ | 499 | CCATAGGTGCCCAACGCTC | 798 | TGAGGGCAAGTTCAGGG |
| 48 | | TGG | | TCCCA |
| crLAG3_ | 500 | CCATAGGTGCCCAACGCTC | 799 | TGAGGGCAAGTTCAGGG |
| 49 | | TGG | | TCCCA |
| crLAG3_ | 501 | CCATAGGTGCCCAACGCTC | 800 | TGAGGGCAAGTTCAGGG |
| 50 | | TGG | | TCCCA |
| crLAG3_ | 502 | CATCCTTCTCCTCCTTCCG | 801 | GACTGGGCTGCTGAGAT |
| 51 | | C | | CTG |
| crLAG3_ | 503 | CATCCTTCTCCTCCTTCCG | 802 | GACTGGGCTGCTGAGAT |
| 52 | | C | | CTG |
| crLAG3_ | 504 | CATCCTTCTCCTCCTTCCG | 803 | GACTGGGCTGCTGAGAT |
| 53 | | C | | CTG |
| crLAG3_ | 505 | GACGGTTGGTGGTCAAGAG | 804 | CACGCTCAGCACCGTGT |
| 54 | | A | | A |
| crLAG3_ | 506 | CGCTACACGGTGCTGAGC | 805 | CACATACTCGAGGCCTG |
| 55 | | | | GC |
| crLAG3_ | 507 | CTGAGATGGGGAGAGGGTG | 806 | TTCCGGAACCAATGCAC |
| 56 | | A | | AGA |
| crPDCD1_ | 508 | TCTCTCAGACTCCCCAGAC | 807 | AGCTTGTCCGTCTGGTT |
| 1 | | AGG | | GCT |
| crPDCD1_ | 509 | CTAAGTCCCTGATGAAGGC | 808 | AGGAAGGAAGGCACAGT |
| 2 | | CCC | | GGATC |
| crPDCD1_ | 510 | GCTGACTCCCTCTCCCTTT | 809 | CGCTAGGAAAGACAATG |
| 3 | | CTC | | GTGGC |
| crPDCD1_ | 511 | TCTCTGTGGACTATGGGGA | 810 | CCAAGAGCAGTGTCCAT |
| 4 | | GCT | | CCTCA |
| crPDCD1_ | 512 | CTGCAGCTTCTCCAACACA | 811 | GAGGTAGGTGCCGCTGT |
| 5 | | TCG | | CATT |
| crPDCD1_ | 513 | GATGTGGAGGAAGAGGGGG | 812 | TACCTAAGAACCATCCT |
| 6 | | C | | GGCCG |
| crPDCD1_ | 514 | CTGCAGCTTCTCCAACACA | 813 | GAGGTAGGTGCCGCTGT |
| 7 | | TCG | | CATT |
| crPDCD1_ | 515 | CTGCAGCTTCTCCAACACA | 814 | GAGGTAGGTGCCGCTGT |
| 8 | | TCG | | CATT |
| crPDCD1_ | 516 | CTGCAGCTTCTCCAACACA | 815 | GAGGTAGGTGCCGCTGT |
| 9 | | TCG | | CATT |
| crPDCD1_ | 517 | CTGCAGCTTCTCCAACACA | 816 | GAGGTAGGTGCCGCTGT |
| 10 | | TCG | | CATT |
| crPDCD1_ | 518 | GCGTGACTTCCACATGAGC | 817 | AGCTCCTGATCCTGTGC |
| 11 | | G | | AG |
| crPDCD1_ | 519 | GCGTGACTTCCACATGAGC | 818 | AGCTCCTGATCCTGTGC |
| 12 | | G | | AG |
| crPDCD1_ | 520 | GCGTGACTTCCACATGAGC | 819 | AGCTCCTGATCCTGTGC |
| 13 | | G | | AG |
| crPDCD1_ | 521 | CTCTAGTCTGCCCTCACCC | 820 | GACCCAGACTAGCAGCA |
| 14 | | CT | | CCAG |
| crPDCD1_ | 522 | CTCTAGTCTGCCCTCACCC | 821 | GACCCAGACTAGCAGCA |
| 15 | | CT | | CCAG |
| crPDCD1_ | 523 | GATGTGGAGGAAGAGGGGG | 822 | TACCTAAGAACCATCCT |
| 16 | | C | | GGCCG |
| crPDCD1_ | 524 | CTCTAGTCTGCCCTCACCC | 823 | GACCCAGACTAGCAGCA |
| 17 | | CT | | CCAG |
| crPDCD1_ | 525 | CTCTAGTCTGCCCTCACCC | 824 | GACCCAGACTAGCAGCA |
| 18 | | CT | | CCAG |
| crPDCD1_ | 526 | CTCTAGTCTGCCCTCACCC | 825 | GACCCAGACTAGCAGCA |
| 19 | | CT | | CCAG |
| crPDCD1_ | 527 | CAGCTCAGGGTAAGCAGCT | 826 | GGTCTTCTCTCGCCACT |
| 20 | | CAT | | GGAAA |
| crPDCD1_ | 528 | CAGCTCAGGGTAAGCAGCT | 827 | GGTCTTCTCTCGCCACT |
| 21 | | CAT | | GGAAA |
| crPDCD1_ | 529 | GCTGACTCCCTCTCCCTTT | 828 | CGCTAGGAAAGACAATG |
| 22 | | CTC | | GTGGC |
| crPDCD1_ | 530 | TCTCTGTGGACTATGGGGA | 829 | CCAAGAGCAGTGTCCAT |
| 23 | | GCT | | CCTCA |
| crPDCD1_ | 531 | GATGTGGAGGAAGAGGGGG | 830 | TACCTAAGAACCATCCT |
| 24 | | C | | GGCCG |
| crPDCD1_ | 532 | GCCACCATTGTCTTTCCTA | 831 | TTCTCCTGAGGAAATGC |
| 25 | | GCG | | GCTGA |
| crPDCD1_ | 533 | GATGTGGAGGAAGAGGGGG | 832 | TACCTAAGAACCATCCT |
| 26 | | C | | GGCCG |
| crPDCD1_ | 534 | TCTCTCAGACTCCCCAGAC | 833 | AGCTTGTCCGTCTGGTT |
| 27 | | AGG | | GCT |
| crPDCD1_ | 535 | TCTCTCAGACTCCCCAGAC | 834 | AGCTTGTCCGTCTGGTT |
| 28 | | AGG | | GCT |
| crPDCD1_ | 536 | TCTCTCAGACTCCCCAGAC | 835 | AGCTTGTCCGTCTGGTT |
| 29 | | AGG | | GCT |
| crPDCD1_ | 537 | TCTCTCAGACTCCCCAGAC | 836 | AGCTTGTCCGTCTGGTT |
| 30 | | AGG | | GCT |
| crPTPN1_ | 538 | TGGTGTCTGTCTTCTGTCA | 837 | TTCTTGTACGAGAGAGC |
| 1 | | GC | | CAGAG |
| crPTPN1_ | 539 | CGAAATGCAGGCAGCAAGC | 838 | CACCCAAATATCACTGG |
| 2 | | TAT | | TGTGGA |
| crPTPN1_ | 540 | CTCTGGGAAAGAAGCAGAG | 839 | GGTAACATCTTGCCAGA |
| 3 | | AA | | CCCA |
| crPTPN1_ | 541 | TTCTGTCTACCTCTGTATG | 840 | GAAATACGACGTTGGTG |
| 1_4 | | TTTGC | | GAGGAG |
| crPTPN1_ | 542 | CTTGGACTAGGCTGGGGAG | 841 | TGGTCAGAAAACACTGT |
| 1_5 | | TA | | GAAAAG |
| crPTPN1_ | 543 | AGGACGTCAGTTTCAAGTC | 842 | GATCAGCCCCTTAACAC |
| 1_6 | | TCTC | | GACTC |
| crPTPN1_ | 544 | TCCAAGCATGGTTTTACCA | 843 | GTTGTTGTGGAAAGTAG |
| 1_7 | | CTTC | | TGCTGA |
| crPTPN1_ | 545 | CGCACACAATTCTGAACAT | 844 | AGGTACAGAGGTGCTAG |
| 1_8 | | TTCC | | GAATC |
| crPTPN1_ | 546 | CCCTTGGAGGAATGTGTCT | 845 | GAACAAAATCTCCAGGG |
| 1_9 | | ACTTTT | | TGGCTC |
| crPTPN1_ | 547 | G+F96AACAAAATCTCCAG | 846 | CCCTTGGAGGAATGTGT |
| 1_10 | | GGTGGCTC | | CTACTTTT |
| crPTPN6_ | 548 | CTCTACTCCTGCACCGACT | 847 | GCGGGTACTTGAGGTGG |
| 1 | | GG | | ATGAT |
| crPTPN6_ | 549 | GGGGGATCAGGTGACCCAT | 848 | GGAGCCCTCACCTCTCA |
| 2 | | A | | CTA |
| crPTPN6_ | 550 | CCCGATGGATGCCCTCTTT | 849 | GAGGGTGGAGACCTGTG |
| 3 | | G | | AGA |
| crPTPN6_ | 551 | GCACAGGCACCATCATTGT | 850 | TGAACTTGTACTGCGCC |
| 4 | | C | | TCC |
| crPTPN6_ | 552 | CGACCCTCCCTTTCCAGAA | 851 | AGAACAAGTCCAGGGAG |
| 5 | | C | | GGA |
| crPTPN6_ | 553 | GATGGTGAGGTAAGGGCCT | 852 | TACCTGACGGAGAGCGA |
| 6 | | G | | GAA |
| crPTPN6_ | 554 | GGCCCCTCTCTGTGAATGT | 853 | ACTGAGCACAGAAAGCA |
| 7 | | C | | CGA |
| crPTPN6_ | 555 | GTGGCCTGGGTCTTACCTT | 854 | CTGCCTTACCTCGCACA |
| 8 | | C | | TGA |
| crPTPN6_ | 556 | GTGGCCTGGGTCTTACCTT | 855 | CTGCCTTACCTCGCACA |
| 9 | | C | | TGA |
| crPTPN6_ | 557 | GTGGCCTGGGTCTTACCTT | 856 | CTGCCTTACCTCGCACA |
| 10 | | C | | TGA |
| crPTPN6_ | 558 | GTGGCCTGGGTCTTACCTT | 857 | CTGCCTTACCTCGCACA |
| 11 | | C | | TGA |
| crPTPN6_ | 559 | GTGGCCTGGGTCTTACCTT | 858 | CTGCCTTACCTCGCACA |
| 12 | | C | | TGA |
| crPTPN6_ | 560 | GTGGCCTGGGTCTTACCTT | 859 | CTGCCTTACCTCGCACA |
| 13 | | C | | TGA |
| crPTPN6_ | 561 | CTGGACGTTTCTTGTGCGT | 860 | GGTCCCCAGCCTTGAAT |
| 14 | | G | | TCA |
| crPTPN6_ | 562 | CTGGACGTTTCTTGTGCGT | 861 | GGTCCCCAGCCTTGAAT |
| 15 | | G | | TCA |
| crPTPN6_ | 563 | GGAGGGTCTGCCTGGGCTT | 862 | GTAGACAAAGGCGCCTG |
| 16 | | GAA | | AGGCC |
| crPTPN6_ | 564 | GATGGTGAGGTAAGGGCCT | 863 | TACCTGACGGAGAGCGA |
| 17 | | G | | GAA |
| crPTPN6_ | 565 | CTGAGGCTCCTGTCTGTGA | 864 | GTAGACAAAGGCGCCTG |
| 18 | | C | | AGG |
| crPTPN6_ | 566 | CTCAAGTCCTGTGAATGGC | 865 | CAGAAGCTCACATCTGG |
| 19 | | CT | | GGG |
| crPTPN6_ | 567 | CTCAAGTCCTGTGAATGGC | 866 | CAGAAGCTCACATCTGG |
| 20 | | CT | | GGG |
| crPTPN6_ | 568 | GACTTCTCGCTCTTCCCCA | 867 | GCAAGGAGGGGAAGGTG |
| 21 | | C | | TC |
| crPTPN6_ | 569 | GACTTCTCGCTCTTCCCCA | 868 | GCAAGGAGGGGAAGGTG |
| 22 | | C | | TC |
| crPTPN6_ | 570 | GACACCTTCCCCTCCTTGC | 869 | CGGTATCCTGGGTGAAT |
| 23 | | | | GGG |
| crPTPN6_ | 571 | CCGATGGATGCCCTCTTTG | 870 | GAGGGTGGAGACCTGTG |
| 24 | | G | | AGA |
| crPTPN6_ | 572 | GCTGATGCTCATTTCCCCA | 871 | GAGGGTGGAGACCTGTG |
| 25 | | C | | AGA |
| crPTPN6_ | 573 | GATGCTCATTTCCCCACCC | 872 | GAGGGTGGAGACCTGTG |
| 26 | | A | | AGA |
| crPTPN6_ | 574 | CTCTCCGCCCACTCCCAGT | 873 | CAGCACAGGCCCTGAAC |
| 27 | | TGA | | CACTG |
| crPTPN6_ | 575 | CTTGCATGGGTGAGGGTGG | 874 | ACCCGGCCTTTCTCCAC |
| 28 | | CAG | | CTCTC |
| crPTPN6_ | 576 | GCTCACTGTCTTGGGGTGC | 875 | TGCCCTGGCATCTGACT |
| 29 | | GTC | | GCTCT |
| crPTPN6_ | 577 | GCTCACTGTCTTGGGGTGC | 876 | TGCCCTGGCATCTGACT |
| 30 | | GTC | | GCTCT |
| crPTPN6_ | 578 | GCTCACTGTCTTGGGGTGC | 877 | TGCCCTGGCATCTGACT |
| 31 | | GTC | | GCTCT |
| crPTPN6_ | 579 | CCCATCCGTCCATCCAACA | 878 | TTCGGTTGTGTCATGCT |
| 32 | | A | | CCC |
| crPTPN6_ | 580 | CCCATCCGTCCATCCAACA | 879 | TTCGGTTGTGTCATGCT |
| 33 | | A | | CCC |
| crPTPN6_ | 581 | CGACCCTCCCTTTCCAGAA | 880 | AGAACAAGTCCAGGGAG |
| 34 | | C | | GGA |
| crPTPN6_ | 582 | GGCCCTACTCTGTGACCAA | 881 | GCCAGATCTCCCGAATC |
| 35 | | C | | AGG |
| crPTPN6_ | 583 | CACGGTAGACAGGAGGCAA | 882 | GCACAAGAGAGTGGCCA |
| 36 | | G | | AAA |
| crPTPN6_ | 584 | GTCGGGTAGGGTGAGATGG | 883 | ATCATCCTCACCTGCAG |
| 37 | | A | | TGC |
| crPTPN6_ | 585 | CCTGATTCGGGAGATCTGG | 884 | AACAGCTCATGGCACTT |
| 38 | | C | | AGC |
| crPTPN6_ | 586 | CCTGATTCGGGAGATCTGG | 885 | AACAGCTCATGGCACTT |
| 39 | | C | | AGC |
| crPTPN6_ | 587 | CCTGATTCGGGAGATCTGG | 886 | AACAGCTCATGGCACTT |
| 40 | | C | | AGC |
| crPTPN6_ | 588 | GCTTGACTGGCCTCTGATG | 887 | TCAATGTCACAGTCCAG |
| 41 | | G | | GCC |
| crPTPN6_ | 589 | GGCCTGGACTGTGACATTG | 888 | AGAGGGACAGTGGGAAG |
| 42 | | A | | GTG |
| crPTPN6_ | 590 | GGCCTGGACTGTGACATTG | 889 | AGAGGGACAGTGGGAAG |
| 43 | | A | | GTG |
| crPTPN6_ | 591 | GGCCTGGACTGTGACATTG | 890 | AGAGGGACAGTGGGAAG |
| 44 | | A | | GTG |
| crPTPN6_ | 592 | CTCTACTCCTGCACCGACT | 891 | GCGGGTACTTGAGGTGG |
| 45 | | GG | | ATGAT |
| crPTPN6_ | 593 | TTCAGGCTTGGTTCTCACC | 892 | CAGGTCAGGAGACAGCA |
| 46 | | C | | CAG |
| crPTPN6_ | 594 | GCCTCTGTCCTCTAGGAGC | 893 | TGACCGCTGCTTCTTCA |
| 47 | | T | | CTT |
| crPTPN6_ | 595 | GCCTCTGTCCTCTAGGAGC | 894 | TGACCGCTGCTTCTTCA |
| 48 | | T | | CTT |
| crPTPN6_ | 596 | CTGTGCTGTCTCCTGACCT | 895 | AAGAGCTGTACCATGGC |
| 49 | | G | | CAC |
| crPTPN6_ | 597 | CTGTGCTGTCTCCTGACCT | 896 | AAGAGCTGTACCATGGC |
| 50 | | G | | CAC |
| crPTPN6_ | 598 | CTGTGCTGTCTCCTGACCT | 897 | AAGAGCTGTACCATGGC |
| 51 | | G | | CAC |
| crPTPN6_ | 599 | ATGGAGGGGAGAAGTTTGC | 898 | GGAGGGGATGGAGGGTA |
| 52 | | G | | GG |
| crPTPN6_ | 600 | GGCCCCTCTCTGTGAATGT | 899 | ACTGAGCACAGAAAGCA |
| 53 | | C | | CGA |
| crTIGIT_ | 601 | AAGAGGCCACATCTGCTTC | 900 | GTGGCATGCTCTTGGAG |
| 1 | | C | | TCT |
| crTIGIT_ | 602 | GGCTCCAGTCCCATGGTTA | 901 | TTCTAGTCAACGCGACC |
| 2 | | C | | ACC |
| crTIGIT_ | 603 | ATGTCACCTCTCCTCCACC | 902 | TCTCCCAGTGTACGTCC |
| 3 | | A | | CAT |
| crTIGIT_ | 604 | CCCAGGACTCACATGTGCT | 903 | GAAGGATGGGGAGATGT |
| 4 | | T | | GCC |
| crTIGIT_ | 605 | ATGTCACCTCTCCTCCACC | 904 | TCTCCCAGTGTACGTCC |
| 5 | | A | | CAT |
| crTIGIT_ | 606 | AAGAGGCCACATCTGCTTC | 905 | GTGGCATGCTCTTGGAG |
| 6 | | C | | TCT |
| crTIGIT_ | 607 | ATGTCACCTCTCCTCCACC | 906 | TCTCCCAGTGTACGTCC |
| 7 | | A | | CAT |
| crTIGIT_ | 608 | ATGTCACCTCTCCTCCACC | 907 | TCTCCCAGTGTACGTCC |
| 8 | | A | | CAT |
| crTIGIT_ | 609 | GGCACATCTCCCCATCCTT | 908 | TGCTGTGCAGTGTTTCA |
| 9 | | C | | GGA |
| crTIGIT_ | 610 | GGCACATCTCCCCATCCTT | 909 | TGCTGTGCAGTGTTTCA |
| 10 | | C | | GGA |
| crTIGIT_ | 611 | GGCACATCTCCCCATCCTT | 910 | TGCTGTGCAGTGTTTCA |
| 11 | | C | | GGA |
| crTIGIT_ | 612 | GGCACATCTCCCCATCCTT | 911 | TGCTGTGCAGTGTTTCA |
| 12 | | C | | GGA |
| crTIGIT_ | 613 | GGTTACACAAAGGGCTTGG | 912 | GCCGGAGCCATTACCTT |
| 13 | | C | | TCT |
| crTIGIT_ | 614 | GTCCTCCCTCTAGTGGCTG | 913 | TCTGGGTCTCTCTCTGG |
| 14 | | A | | GTG |
| crTIGIT_ | 615 | GTCCTCCCTCTAGTGGCTG | 914 | TCTGGGTCTCTCTCTGG |
| 15 | | A | | GTG |
| crTIGIT_ | 616 | AGCTGTAACGCGGTTGAGA | 915 | CCATTCCTCCTGTCCAG |
| 16 | | A | | CTG |
| crTIGIT_ | 617 | AGCTGTAACGCGGTTGAGA | 916 | CCATTCCTCCTGTCCAG |
| 17 | | A | | CTG |
| crTIGIT_ | 618 | AGTTTGCTGGTGTGCATGT | 917 | CATGCAGCTCGGCACAG |
| 18 | | GTGT | | TCCTC |
| crTIGIT_ | 619 | AGTTTGCTGGTGTGCATGT | 918 | CATGCAGCTCGGCACAG |
| 19 | | GTGT | | TCCTC |
| crTIGIT_ | 620 | AGTTTGCTGGTGTGCATGT | 919 | CATGCAGCTCGGCACAG |
| 20 | | GTGT | | TCCTC |
| crTIGIT_ | 621 | AGTTTGCTGGTGTGCATGT | 920 | CATGCAGCTCGGCACAG |
| 21 | | GTGT | | TCCTC |
| ctTIGIT_ | 622 | AGAAGAAAGCCCTCAGAAT | 921 | TGCAGTTACCCAGGCTT |
| 22 | | CCA | | CTG |
| crTIGIT_ | 623 | TGTGGAAGGTGACCTCAGG | 922 | AGAAGATGCCTCTGGTT |
| 23 | | A | | GCT |
| crTIGIT_ | 624 | GGAGGAGCAACAGGATGGA | 923 | TGGTGGAGGAGAGGTGA |
| 24 | | C | | CAT |
| crTIGIT_ | 625 | GAAGCTGTGTCCAGGCAGA | 924 | CGCAGCACTGATGGAGA |
| 25 | | A | | GTA |
| crTIGIT_ | 626 | GAAGCTGTGTCCAGGCAGA | 925 | CGCAGCACTGATGGAGA |
| 26 | | A | | GTA |
| crTIGIT_ | 627 | GGAGGAGCAACAGGATGGA | 926 | TGGTGGAGGAGAGGTGA |
| 27 | | C | | CAT |
| crTIGIT_ | 628 | CCCAGGACTCACATGTGCT | 927 | GAAGGATGGGGAGATGT |
| 28 | | T | | GCC |
| crTIGIT_ | 629 | CCCAGGACTCACATGTGCT | 928 | GAAGGATGGGGAGATGT |
| 29 | | T | | GCC |
| crTIGIT_ | 630 | CCCAGGACTCACATGTGCT | 929 | GAAGGATGGGGAGATGT |
| 30 | | T | | GCC |
| crTIGIT_ | 631 | ATGTCACCTCTCCTCCACC | 930 | TCTCCCAGTGTACGTCC |
| 31 | | A | | CAT |
| crTIM3_ | 632 | GGCCATCCTTGTATCTCTC | 931 | GCGGCTACTGCTCATGT |
| 1 | | CC | | GAT |
| crTIM3_ | 633 | GCACGGAGATATCCATGCC | 932 | GACATTAGCCAAGGTCA |
| 2 | | T | | CCC |
| crTIM3_ | 634 | GGCCATCCTTGTATCTCTC | 933 | GCGGCTACTGCTCATGT |
| 3 | | CC | | GAT |
| crTIM3_ | 635 | TGTCTCCACCACTTCCCTC | 934 | ACATTAGCCAAGGTCAC |
| 4 | | T | | CCC |
| crTIM3_ | 636 | GATCCGGCAGCAGTAGATC | 935 | ATGCCTATCTGCCCTGC |
| 5 | | C | | TTC |
| crTIM3_ | 637 | CCCTTGTCCTCTGTACAGC | 936 | GCGGCTACTGCTCATGT |
| 6 | | A | | GAT |
| crTIM3_ | 638 | TCTCCTTTGCGGAAATCCC | 937 | ATGCAGGGTCCTCAGAA |
| 7 | | C | | GTG |
| crTIM3_ | 639 | GATCCGGCAGCAGTAGATC | 938 | ATGCCTATCTGCCCTGC |
| 8 | | C | | TTC |
| crTIM3_ | 640 | GATCCGGCAGCAGTAGATC | 939 | ATGCCTATCTGCCCTGC |
| 9 | | C | | TTC |
| crTIM3_ | 641 | GATCCGGCAGCAGTAGATC | 940 | ATGCCTATCTGCCCTGC |
| 10 | | C | | TTC |
| crTIM3_ | 642 | GATCCGGCAGCAGTAGATC | 941 | ATGCCTATCTGCCCTGC |
| 11 | | C | | TTC |
| crTIM3_ | 643 | GATCCGGCAGCAGTAGATC | 942 | ATGCCTATCTGCCCTGC |
| 12 | | C | | TTC |
| crTIM3_ | 644 | GCAAATGTCCACTCACCTG | 943 | GGAGCCTGTCCTGTGTT |
| 13 | | G | | TGA |
| crTIM3_ | 645 | GCAAATGTCCACTCACCTG | 944 | GGAGCCTGTCCTGTGTT |
| 14 | | G | | TGA |
| crTIM3_ | 646 | TCTTAGTGGCCCTCCTCCA | 945 | CGCAAAGGAGATGTGTC |
| 15 | | G | | CCT |
| crTIM3_ | 647 | CCCTTGTCCTCTGTACAGC | 946 | GCGGCTACTGCTCATGT |
| 16 | | A | | GAT |
| crTIM3_ | 648 | TCTTAGTGGCCCTCCTCCA | 947 | CGCAAAGGAGATGTGTC |
| 17 | | G | | CCT |
| crTIM3_ | 649 | TCTTAGTGGCCCTCCTCCA | 948 | CGCAAAGGAGATGTGTC |
| 18 | | G | | CCT |
| crTIM3_ | 650 | ACTGAGCATCACCAATGGG | 949 | CAGTGGGATCTACTGCT |
| 19 | | G | | GCC |
| crTIM3_ | 651 | GTCCCCTGGTGGTAAGCAT | 950 | ACGTAGGTATCCAGGCA |
| 20 | | C | | GGT |
| crTIM3_ | 652 | GTCCCCTGGTGGTAAGCAT | 951 | ACGTAGGTATCCAGGCA |
| 21 | | C | | GGT |
| crTIM3_ | 653 | AAAGATTCCCTCCTCTGCC | 952 | AGGTTTGGAAGCTGAGG |
| 22 | | C | | GTG |
| crTIM3_ | 654 | GCCAGCTAAAGATTCCCTC | 953 | CTTGCTGCCCCTTTGAT |
| 23 | | CT | | TCC |
| crTIM3_ | 655 | GCACGGAGATATCCATGCC | 954 | TGTTTCTGACATTAGCC |
| 24 | | T | | AAGGT |
| crTIM3_ | 656 | CCCTTGTCCTCTGTACAGC | 955 | GCGGCTACTGCTCATGT |
| 25 | | A | | GAT |
| crTIM3_ | 657 | TGAGTACAACATAGCTCAC | 956 | CGGAGTAGAATTCATTT |
| 26 | | AAA | | CAAATAGG |
| crTIM3_ | 658 | TGAGTACAACATAGCTCAC | 957 | CGGAGTAGAATTCATTT |
| 27 | | AAA | | CAAATAGG |
| crTIM3_ | 659 | CAAGGACAAGGTGGGCATG | 958 | TCCTCTCTCTCTCTCTC |
| 28 | | AAG | | TCTCTCT |
| crTIM3_ | 660 | CACAGATCCCTGCTCCGAT | 959 | AGGACTCAGCCATCCTG |
| 29 | | G | | TGA |
| crTIM3_ | 661 | CACAGATCCCTGCTCCGAT | 960 | AGGACTCAGCCATCCTG |
| 30 | | G | | TGA |
| crTIM3_ | 662 | CGCCGAAGATAAGAGCCAG | 961 | CAGCCATCCTGTGATGT |
| 31 | | A | | TGT |
| crTIM3_ | 663 | GGATTTGGATGGACAAAAG | 962 | TGGCCAATGACTTACGG |
| 32 | | GGT | | GAC |
| crTIM3_ | 664 | GGATTTGGATGGACAAAAG | 963 | TGGCCAATGACTTACGG |
| 33 | | GGT | | GAC |
| crTIM3_ | 665 | CAAAGCCCCAGGACAGGAT | 964 | GCGTGCTTCCAGTGAAC |
| 34 | | T | | CTA |
| crTIM3_ | 666 | CAAAGCCCCAGGACAGGAT | 965 | GCGTGCTTCCAGTGAAC |
| 35 | | T | | CTA |
| crTIM3_ | 667 | CCCTTGTCCTCTGTACAGC | 966 | GCGGCTACTGCTCATGT |
| 36 | | A | | GAT |
| crTIM3_ | 668 | CAAAGCCCCAGGACAGGAT | 967 | GCGTGCTTCCAGTGAAC |
| 37 | | T | | CTA |
| crTIM3_ | 669 | CAAAGCCCCAGGACAGGAT | 968 | GCGTGCTTCCAGTGAAC |
| 38 | | T | | CTA |
| crTIM3_ | 670 | CAAAGCCCCAGGACAGGAT | 969 | GCGTGCTTCCAGTGAAC |
| 39 | | T | | CTA |
| crTIM3_ | 671 | CATTGGGCTCCTCCACTTC | 970 | GCTGTCTCTTTGGGAAA |
| 40 | | A | | GCC |
| crTIM3_ | 672 | CATTGGGCTCCTCCACTTC | 971 | GCTGTCTCTTTGGGAAA |
| 41 | | A | | GCC |
| crTIM3_ | 673 | CATTGGGCTCCTCCACTTC | 972 | GCTGTCTCTTTGGGAAA |
| 42 | | A | | GCC |
| crTIM3_ | 674 | CATTGCAAAGCGACAACCC | 973 | CCGTGTTACCTGGGAAA |
| 43 | | A | | TGC |
| crTIM3_ | 675 | CATTGCAAAGCGACAACCC | 974 | CCGTGTTACCTGGGAAA |
| 44 | | A | | TGC |
| crTIM3_ | 676 | CATTGCAAAGCGACAACCC | 975 | CCGTGTTACCTGGGAAA |
| 45 | | A | | TGC |
| crTIM3_ | 677 | CATTGCAAAGCGACAACCC | 976 | CCGTGTTACCTGGGAAA |
| 46 | | A | | TGC |
| crTIM3_ | 678 | CAGTGCAGGTCCCAGTTCA | 977 | AGTGGAGGAGCCCAATG |
| 47 | | A | | AGT |
| crTIM3_ | 679 | CAGTGCAGGTCCCAGTTCA | 978 | AGTGGAGGAGCCCAATG |
| 48 | | A | | AGT |
| crTIM3_ | 680 | TCAAACACAGGACAGGCTC | 979 | AACAGGACTGCAGCAGT |
| 49 | | C | | AGC |
| crTIM3_ | 681 | TCTCCTTTGCGGAAATCCC | 980 | ATGCAGGGTCCTCAGAA |
| 50 | | C | | GTG |
| crTIM3_ | 682 | TCTCCTTTGCGGAAATCCC | 981 | ATGCAGGGTCCTCAGAA |
| 51 | | C | | GTG |
| crAAVS1 | 683 | CATCTCTCCTCCCTCACCC | 982 | AAGAGGATGGAGAGGTG |
| | | A | | GCT |

### Example 10: NGS Data Analysis

Initial quality assessment of the obtained reads was performed with FastQC36. The sequencing data were aligned and analyzed with the CRISPResso2 software, using CRISPRessoBatch command with the parameters --cleavage_offset 1 --quantification_window_size 10 -- --quantification_window_center 1 --expand_ambiguous_alignments for the INDEL frequency analysis. For the ORF disruption analysis, CRISPRessoBatch command with the parameters --cleavage_offset 1 -coding_seq <EXON_SEQ> --quantification_window_size 0 --quantification_window_center 1 --expand_ambiguous_alignments was used. Modification rates from the CRISPResso2 software output were analyzed in Excel.

### Example 11: CRISPR-MAD7 Platform for Human Genome Editing Using the Jurkat T-Cell Leukemia Cell Line

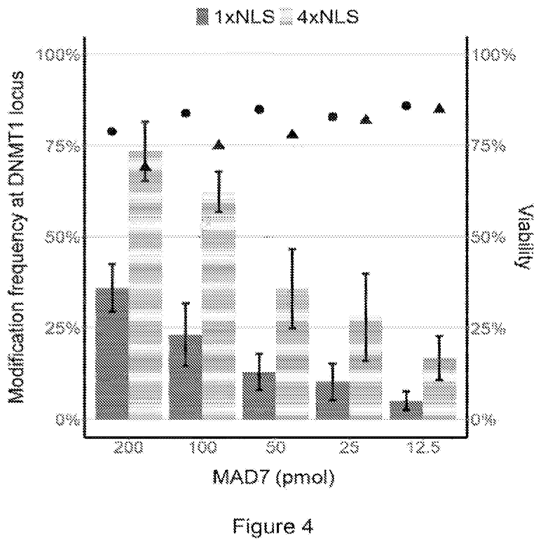

MAD7 nuclease comprising a His6 tag and either one (MAD7-1NLS) or four (MAD7-4NLS) nuclear localization signals (NLS) were used (FIG. 4). RNPs were generated as described in Example 3. Editing frequency of the MAD7 nuclease complexed with one or more guide nucleic acids comprising a spacer sequence of SEQ ID NOs: 86-384 as shown in Table 5 was determined by nucleofection of RNPs in Jurkat T-cells using the Lonza recommended nucleofection program SE-CL-120 (Example 5), followed by genomic DNA extraction (Example 8), amplification of the edited locus and targeted next-generation sequencing (Example 9) for identification of the edits, and finally by computational analysis (Example 10) of modification frequency using the CRISPResso2 algorithm.

Firstly, using a gNA targeting the DNMT1 locus, the editing frequency of MAD7 comprising either one or four NLS complexed with the respective gNA was compared. RNP concentration-dependent modification efficiency was observed as evidenced by an increased fraction of modified amplicons (FIG. 4, left axis, dark grey for MAD7-1NLS and light grey representing MAD7-4NLS). Error bars represent one standard deviation for a sample of 3 (n=3). In this experiment, editing frequency was enhanced in Jurkat cells when treated with RNPs comprising MAD-4NLS, which indicates that optimization of the NLS can improve editing efficiency. A slight decrease in cell viability was seen at higher concentrations of RNP for those comprising four NLS as compared to one NLS (FIG. 4, right axis). Specifically, FIG. 4 shows editing frequency at the DNMT1 locus (n=3; Mean±SD) and cell viability of T-cell leukemic cells as a function of MAD7 comprising one or four nuclear localization signal (NLS) and MAD7-RNP amounts (pmol; constant ratio of 1:1.5 MAD7:gNA). Dark grey bars and circles represent mean modification frequency and viability using MAD7-1NLS, respectively. Light grey bars and triangles represent mean modification frequency and viability using MAD7-4NLS, respectively.

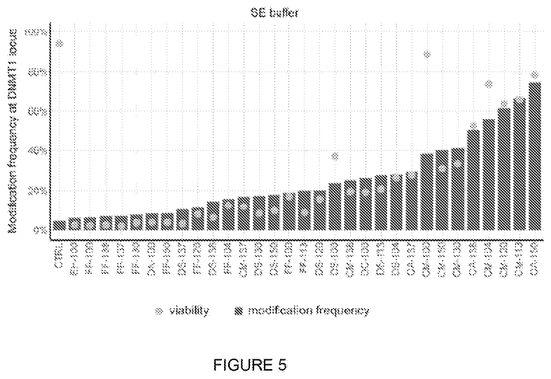

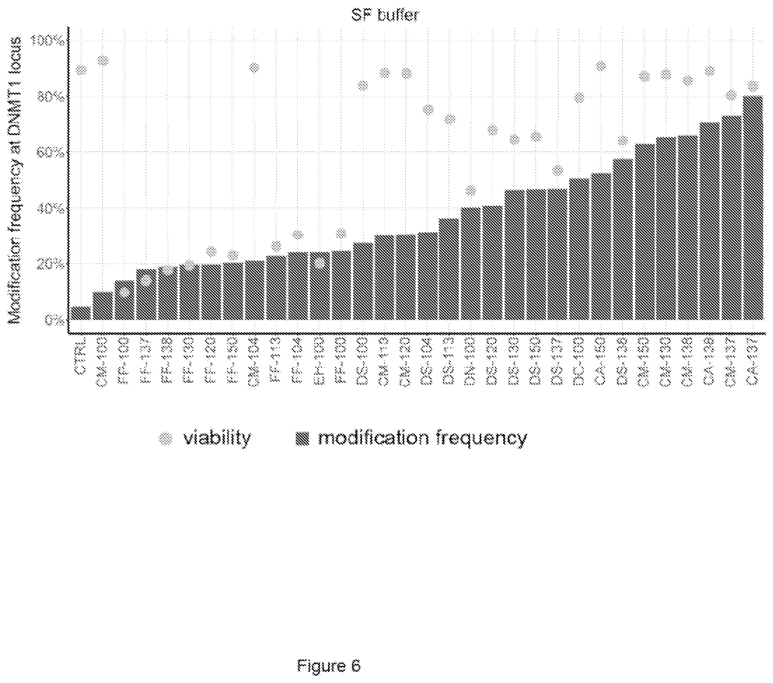

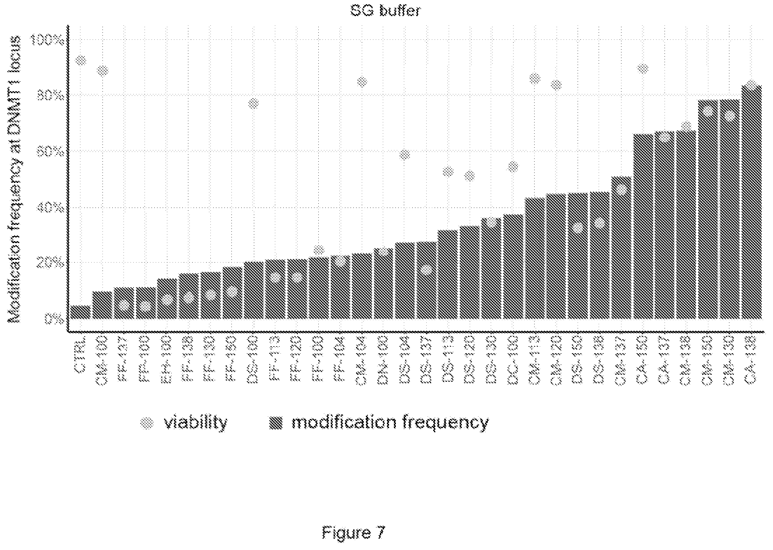

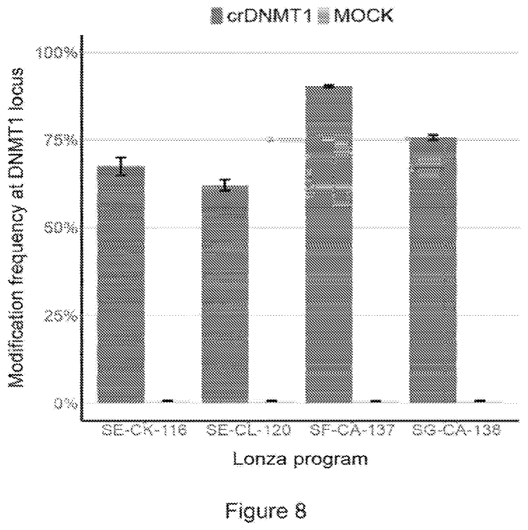

To optimize editing activity, 93 different transfection conditions were tested; 31 nucleofection programs in combination with three buffers-on the Lonza Nucleofector 96-well Shuttle System (FIGS. 5-7). FIGS. 5, 6, and 7 show the editing frequency (bars; x-axis) of each of the electroporation conditions (buffers SE, SF, and SG respectively) as compared to a control (y-axis, control at the top). The majority of buffer-program transfection combinations resulted in suboptimal viability (dots; x-axis) and editing frequency, however, the analysis revealed several conditions that supported substantial rates of both cell viability and editing. Two improved conditions observed in the screen, namely SF-CA-137 and SG-CA-138, were then validated and compared to the Lonza recommended nucleofection programs for T-cell leukemia, namely SE-CL-120 and SE-CK-116 (FIG. 8). Specifically, FIG. 8 shows editing frequency at the DNMT1 locus (n=4; Mean±SD) in T-cell leukemic cell line achieved by utilization of the transfection conditions identified in FIG. 4 (100 pmol MAD7-4NLS) and Lonza recommended nucleofection programs SE-CK-116 and SE-CL-120, as well as the two best nucleofection programs observed in this study, SF-CA-137 and SG-CA-138 (FIGS. 5-7). Dark grey bars represent mean modification frequency using crDNMT1. Light grey bars represent mean modification frequency using crIDTneg (Integrated DNA Technologies, IDT).

### Example 12: Scalable High-Level MAD7-RNP Editing of Immunologically Relevant Genes in Jurkat T-Cell Leukemia Cell Line

The Jurkat T-cell leukemia cell line was used as a model system to screen GNAs demonstrating high editing efficiency. The screen included 298 unique gNAs comprising one or more spacer sequences of SEQ ID NOs: 86-384 of Table 5 targeting the immune checkpoint receptors PDCD1, TIM3, LAG3, TIGIT, and CTLA4, the checkpoint phosphatases PTPN6 (SHP-1) and PTPN11 (SHP-2), and the TCR signaling subunit CD247 (CD3ζ). RNPs were generated as described in Example 3, nucleofected (Example 5), genomic DNA was extracted (Example 8), the edited loci amplified and sequenced (Example 9), and the sequencing data computationally analyzed (Example 10) using the CRISPResso2 algorithm.

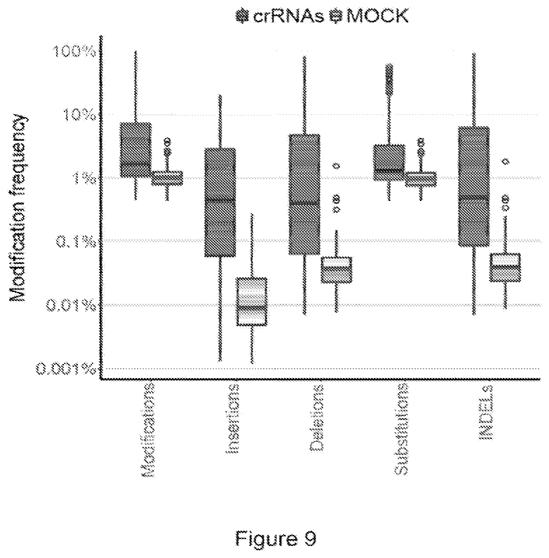

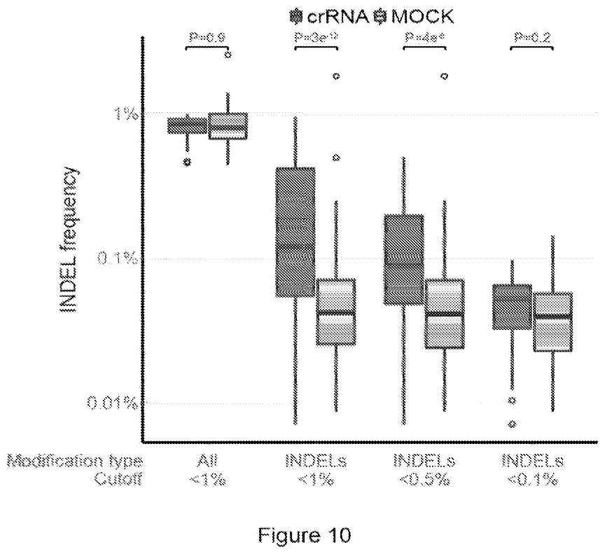

CRISPResso2 software reports the frequency of modifications (insertions, deletions, and substitutions) within a quantification window flanking the position of MAD7-induced cleavage in the amplicon sequence. To better understand detection of editing events, the type of modifications detected in 230 amplicons that were sequenced in both gNA-treated and MOCK samples (no MAD7) were compared. Relatively high modification frequencies (median 1%) in MOCK reactions were observed as a result of high frequency of substitutions (FIG. 9, light grey bars); substitutions were detected at a median frequency of 0.96%, likely due to the errors in NGS base calling or substitutions arising during DNA amplification, while insertions and deletions were found at a much lower median frequency of 0.003% and 0.042%, respectively. Specifically, FIG. 9 shows editing frequency at eight different loci using 298 gNAs (n=3; Mean±SD) in T-cell leukemic cell line as a function of various editing types: all modifications, only insertions, only deletions, only substitutions, or insertions and deletions (INDELs). Edits were achieved using the transfection conditions identified in Example 11, FIG. 4 (100 pmol MAD7-4NLS) and one of the tested Lonza nucleofection programs (FIG. 8; SF-CA-137). Dark grey boxplots represent mean modification frequency using gNAs. Light grey boxplots represent mean modification frequency using crIDTneg (IDT). Thus, the frequency of both insertions and deletions (INDEL) were used as a means to quantify the editing activity of the CRISPR-MAD7 system to minimize low end noise. Moreover, low INDEL frequencies in MOCK reactions enabled sensitive detection of editing events at a significantly greater fraction of sites (Fisher exact test, P=3×10⁻¹²; FIG. 10). Analysis of gNAs with low INDEL frequencies showed statistically significant editing in gNA-treated samples compared to MOCK samples at INDEL frequencies as low as 0.5% (Fisher exact test, P=4×10⁻⁸; FIG. 10). This indicates the sensitivity of the assay to detect modifications in the sub-1% range. Specifically, FIG. 10 shows INDEL frequency at eight different loci using 298 gNAs (n=3; Mean±SD) in T-cell leukemic cell line as a function of two modification types: all modifications<1%, and INDELs<1%, or <0.5%, or <0.1%, with lower INDEL frequencies in MOCK compared to gNA reactions at INDELs<1% (Fisher's exact test; P=3×10⁻¹²) and <0.5% (Fisher exact test, P=4×10⁻⁸). Dark grey boxplots represent mean INDEL frequency using gNAs. Light grey boxplots represent mean INDEL frequency using crIDTneg (IDT).

Since MAD7 can target a wide range of PAM, gNAs adjacent to all YTTN PAM variants were screened and editing specificity of MAD7 in Jurkat cells was analyzed. MAD7 demonstrated editing with all eight combinations of YTTN PAM; in this experiment, editing was higher at the YTTV and TTTV consensus sequences (Fisher exact test; P=2×10⁻³ and P=2×10⁻⁴, respectively). While the majority of highly-active (>50% INDEL frequency) gNAs were found at sites with YTTV and TTTV PAMs, moderately-active (>10% INDEL frequency) gNAs were found to target every PAM sequence with the exception of CTTT. This indicates that MAD7 can edit a wide range of target PAMs, albeit at reduced frequencies (FIG. 11). Specifically, FIG. 11 shows INDEL frequency at eight different loci using 298 gNAs (n=3; Mean±SD) in T-cell leukemic cell line as a function of eight YTTN PAM combinations, and TTTV, YTTN, and YTTV PAM motifs. A grey zone on the plot represents moderately-active gNAs (10-50% INDELs), the zone above highly-active gNAs (>50% INDELs), and the zone below active gNAs (1-10% INDELs). INDEL frequency at the YTTV and TTTV PAM motif is significantly higher compared to YTTN motif (Fisher exact test, P=2×10⁻³ and P=2×10⁻⁴, respectively).

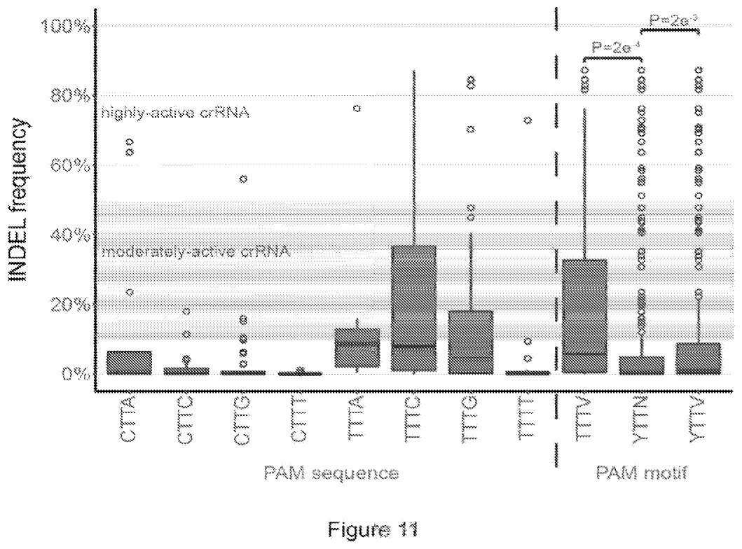

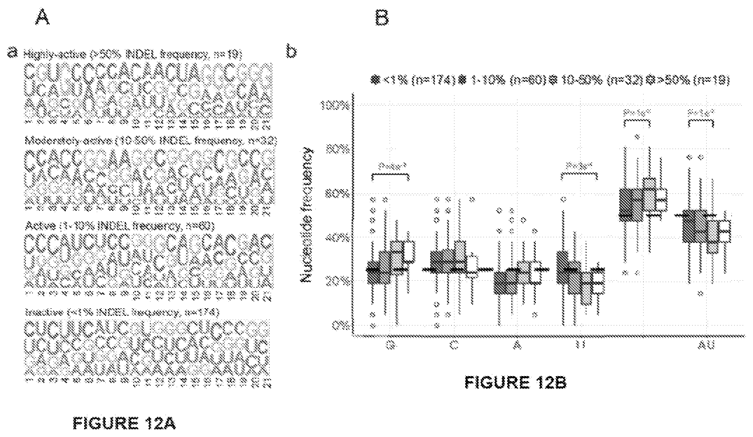

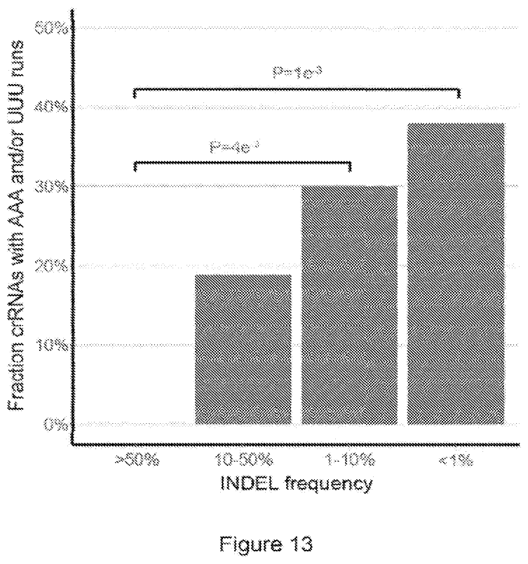

Given the large number of gNAs analyzed, it was determined if the targeted DNA sequence biases editing efficiency. Sequence logos were made to compare the DNA-complementary gNA sequences of inactive (<1% INDELs), active (1-10% INDELs), moderately-active (10-50% INDELs), and highly-active (>50% INDELs) gNAs (FIG. 12A). While there were no strong biases for ribonucleotides at specific positions were identified in this experiment, guanine appeared overrepresented and uracil underrepresented on moderately-active and highly-active gNAs. Next, the frequency of ribonucleotide bases were analyzed within the same four classes of gNAs (FIG. 12B). The analysis confirmed significant enrichment of guanine and depletion of uracil on highly-active gNAs. Specifically, FIG. 12 shows (A) sequence logos comparing DNA-complementary gNA sequences of highly-active (>50% INDELs), moderately-active (10-50% INDELs), active (1-10% INDELs), and inactive (<1% INDELs) gNAs show no strong biases for ribonucleotides at specific positions, however, guanine appeared overrepresented and uracil underrepresented on highly-active and moderately-active gNAs; (B) nucleotide frequency on inactive (<1% INDELs; dark grey box), active (1-10% INDELs; medium grey box), moderately-active (10-50% INDELs; light grey box), and highly-active (>50% INDELs; white box) gNAs, with significant enrichment of guanine and depletion of uracil on highly-active gNAs compared to inactive gNAs (Fisher exact test, P=4×10⁻³ and P=3×10⁻⁴, respectively). Also, significant enrichment of guanine-cytosine content and depletion of adenine-uracil content was observed on moderately-active gNAs compared to inactive gNAs (Fisher exact test, P=1×10⁻²). Moreover, the data showed that nearly 40% of inactive gNAs had runs of three or more adenine or uracil ribonucleotides, while none of the highly-active and <20% of moderately-active gNAs contained such runs (FIG. 13). These sequence features can act as an algorithm for selecting putative high-activity gNAs during initial rounds of screening, and could reduce the overall cost of identifying gNAs for various genes of interest. Specifically, FIG. 13 shows fraction of gNAs with AAA and/or UUU runs as a function of INDEL frequency of highly-active (>50% INDELs), moderately-active (10-50% INDELs), active (1-10% INDELs), and inactive (<1% INDELs) gNAs. Fraction of inactive (<1% INDELs) and active (1-10% INDELs) gNAs containing such runs is higher compared to highly-active (>50% INDELs) gNAs (Fisher exact test, P=1×10⁻³ and P=4×10⁻⁴, respectively).

### Example 13: Validation of gNAs for Gene Editing and Disruption of Immunologically Relevant Genes Using T-Cell Leukemia Cell Line

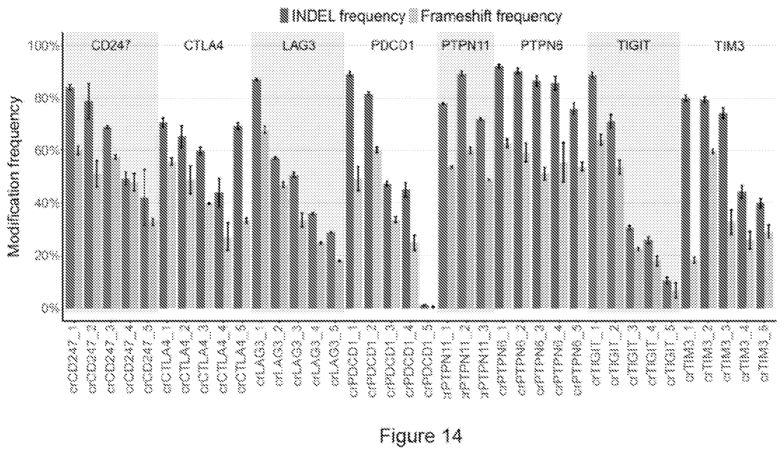

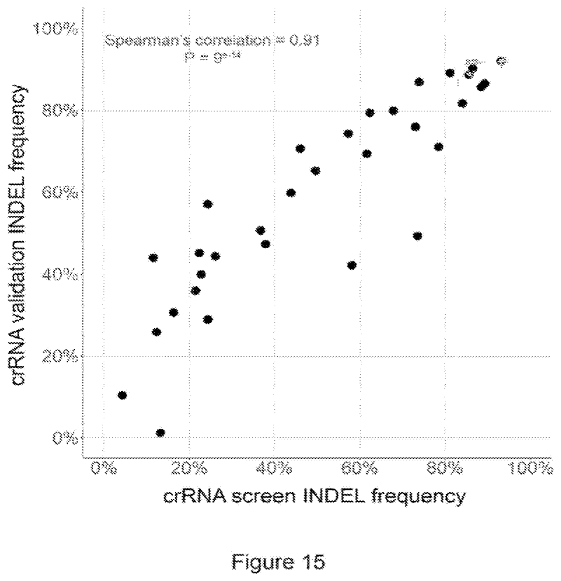

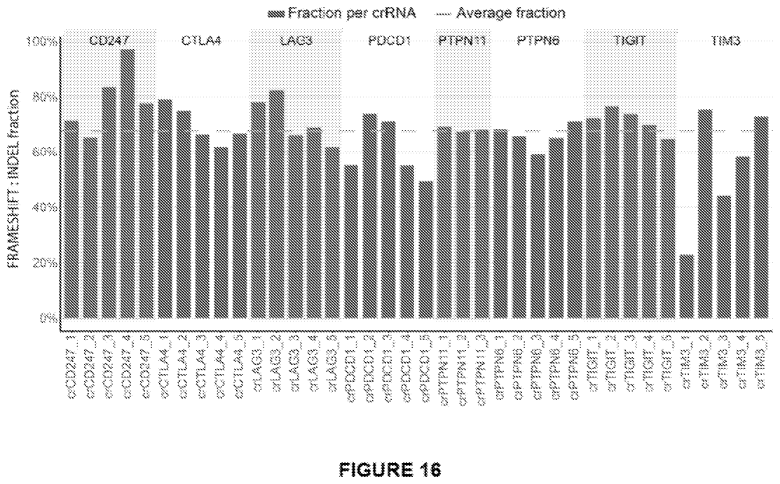

High-efficiency gNAs identified in our initial analysis were validated by assaying INDEL frequency for the top three or five gNAs for each of the selected immunologically relevant genes (FIG. 14). Specifically, FIG. 14 shows INDEL (dark grey bars) and frameshift (light grey bars) frequencies (n=3; Mean±SD) in T-cell leukemic cell line as a function of 38 high-efficiency gNAs. Alternating grey and white zones on the plot represent groups of three to five high-efficiency gNAs per locus. In the validation experiment, the INDEL frequency was significantly correlated to the measurements from the initial screen, highlighting the reproducibility of the INDEL assay (FIG. 15). Specifically, FIG. 15 shows correlation of INDEL frequency in the gNA validation experiment versus INDEL formation in the gNA screen experiment (Spearman's correlation=0.91; P=9×10⁻¹⁴), highlighting reproducibility of the INDEL assay. Using the CRISPresso2 software, the degree of open reading frame (ORF) disruption for each of the validated gNAs was estimated (FIG. 14). In addition, for four high-efficiency gNAs targeting three different exons at the PDCD1 locus, surface expression of the PDCD1 protein was measured by flow cytometry 4, 7, and 11 days post-transfection (data not shown). The data revealed that the protein surface expression after transfection with crPDCD1_2, a gNA targeting the PDCD1 gene at the extracellular domain of the protein, was as low as 10% 4 days post-transfection and remained at this level even at day 11 post-transfection. The surface expression after transfection with the remaining three gNAs was significantly higher, 35% and 85% after transfection with crPDCD1 3 and both crPDCD1 4 and crPDCD1_5, respectively. This is in line with the ORF data analysis, which showed that for most of the gNAs including the high-efficiency crPDCD1s, the predicted number of INDELs leading to frameshifts was similar to that expected from an unbiased DNA repair process, with frameshifts in two-thirds of the edited loci (FIG. 16). However, several of the gNAs had a markedly different degree of ORF disruption; crCD247_4 resulted in frameshifts with 97% frequency, while crTIM3_1 and crTIM3_3 resulted in frameshifts with 23% and 44% frequency, respectively (FIG. 16). Specifically, FIG. 16 shows fraction of frameshift to INDEL frequency (dark grey bars) in T-cell leukemic cell line as a function of 38 high-efficiency gNAs. Average fraction of INDELs leading to frameshifts (dashed line) is approx. 66%. Alternating grey and white zones on the plot represent groups of three to five high-efficiency gNAs per locus. The analysis of repair products indicates that in the case of crTIM3_1, and to some extent crTIM3_3, the bias arose from directly repeated sequences at the DNA cleavage site, which possibly promoted microhomology-mediated end joining (MMEJ) repair following DNA cleavage. These data help inform selection of gNAs for gene KO since some gNAs, such as crTIM3_1, have much lower frequency of gene disruption than would be predicted based on the frequency of INDEL formation.

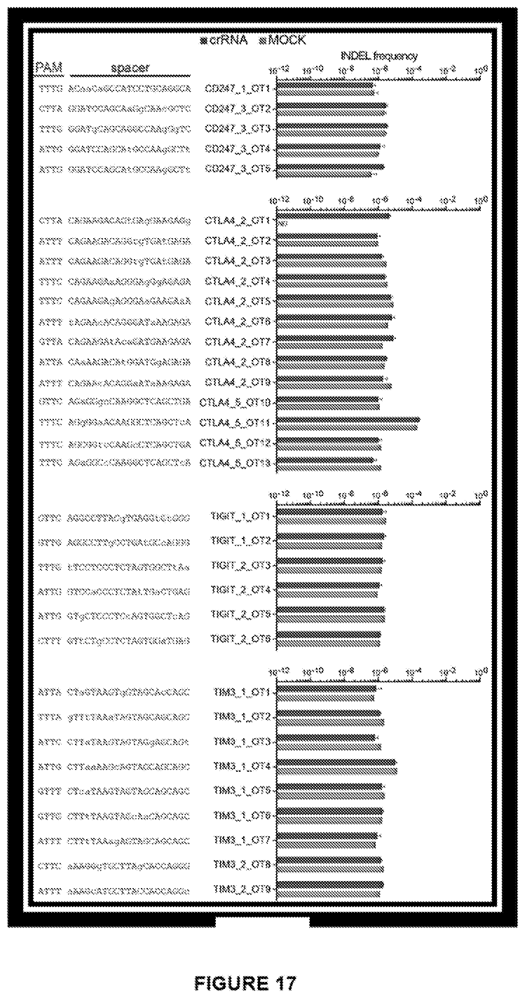

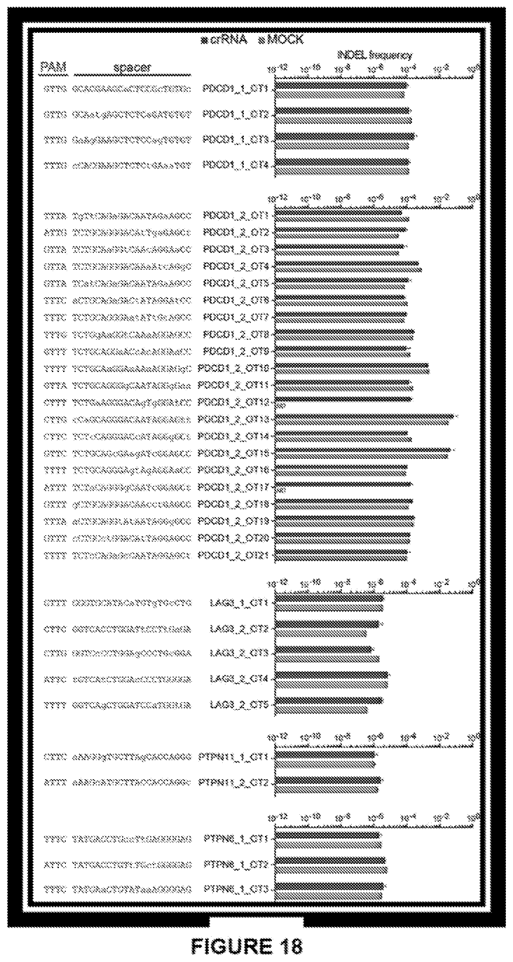

Another consideration for selecting gNAs is the potential for off-target cleavage events. The list of validated gNAs was analyzed using the CasOFFinder software to predict potential off-target editing sites in the genome with up to four mismatches between the gNA and the target DNA sequence. Using the Bioconductor R packages, the predicted off-target sites were matched with the human gene database, and those sites that targeted exons and introns within the genes were extracted. Afterwards, the degree of editing activity at these sites was examined by targeted next-generation sequencing, more specifically, at 25 predicted off-target sites for the top-two PDCD1 gNAs, i.e., crPDCD1_1 and crPDCD1_2. The analysis revealed low-level off-target activity at crPDCD1_2_13 and crPDCD1_2_15 sites, however, INDEL formation at these two sites was statistically insignificant compared to MOCK samples (non-targeting gNAs) (Pairwise T-test, P≥0.05; FIGS. 17 and 18). INDEL frequency at 43 putative off-target sites with up to three mismatches between gNA and target DNA sequence were assayed for the top-two gNAs targeting seven remaining genes (i.e., TIM3, LAG3, TIGIT, CTLA4, PTPN6, PTPN11, and CD247; spacer sequences in Table 5). The analysis revealed no detectable activity at any of the putative off-target sites (FIGS. 17 and 18), which confirms the high cleavage fidelity of MAD7-gNA complexes. Specifically, FIGS. 17-18 show INDEL frequency of MAD7 (n=3; Mean±SD) in T-cell leukemic cell line at predicted off-target sites analyzed by targeted deep sequencing. For crPDCD1, INDEL frequency was analyzed at the putative off-target editing sites with ≤4 mismatches between the gNA and target DNA sequence, and with ≤3 mismatches on the remaining gNAs. PAM sequences and spacer sequences with mismatches marked in red are displayed next to their respective measured INDEL frequencies. No significant INDEL frequency at any of the off-target sites was detected (Pairwise T-test, P≥0.05).

### Example 14: Transgene Insertion in T-Cell Leukemia Cell Line and Primary T-Cells with CRISPR-MAD7 Platform

Insertion of exogenous transgenes is an important aspect of mammalian cell engineering. Gene insertion with CRISPR-Cas is achieved by homology-directed repair of CRISPR-induced DNA breaks using HDR-donor templates to copy exogenous genetic sequences into targeted DNA loci. Several studies indicate that HDR templates, composed of linear double stranded DNA, provide the most robust and efficient method of transgene insertion using CRISPR-Cas genome editing systems.

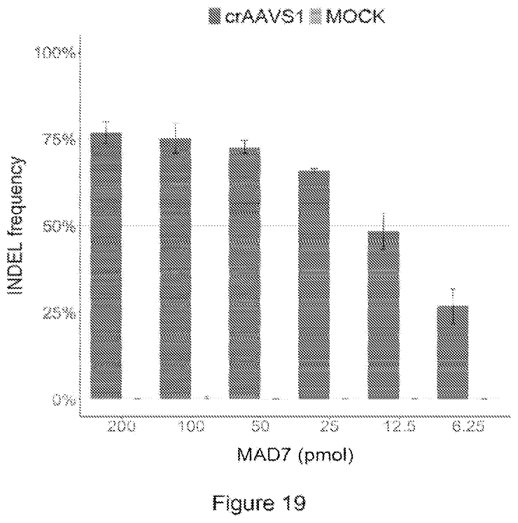

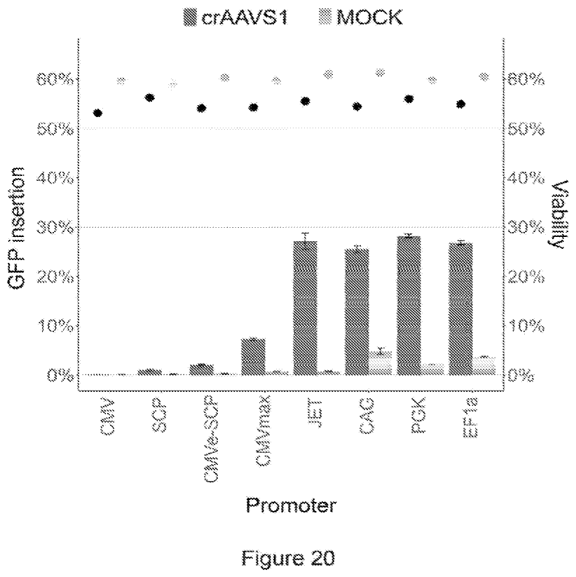

The Jurkat T-cell leukemia cell line was used to evaluate the transgene insertion and expression efficiency using CRISPR-MAD7 RNP complexes. A highly active gNA targeting the AAVS1 (spacer sequence in Table 5) safe-harbor locus (FIG. 19) was used in combination with eight different HDR-repair templates flanked with symmetric homology arms (HA) of 500 base pairs (bp) in the amount of 0.5 μg μL⁻¹. Specifically, FIG. 19 shows INDEL frequency at the AAVS1 locus (n=3; Mean±SD) in T-cell leukemic cell line as a function of MAD7-RNP amounts (pmol; constant ratio of 1:1.5 MAD7:gNA). Dark grey bars represent mean INDEL frequency using crAAVS1. Light grey bars represent mean modification frequency using crIDTneg (IDT). The HDR inserts comprised eight promoters (Table 4) differing in both size and promoter strength to drive GFP expression (FIG. 20). When the transient GFP expression diminished at day 14 post-transfection, comparable insertion efficiencies were observed with stable GFP expressions of up to 30% using four (JET, PGK, EF1a, and CAG) out of eight promoters (FIG. 20), suggesting that the insert size has not affected the integration efficiency at AAVS1 in human T-cell leukemia cell line. Specifically, FIG. 20 shows GFP insertion efficiency at AAVS1 (n=3; Mean±SD) and cell viability of T-cell leukemic cell line measured at day 14 post-transfection. HDR templates consisting of eight different promoters and flanked with symmetric homology arms of 500 base pairs in the amount of 0.5 μg μL⁻¹ were used. Size of promoters in base pairs: CMV, 1400; SCP, 970; CMVe-SCP, 1270; CMVmax, 1830; JET, 1100; CAG, 2600; PGK, 1410; EF-1α, 2090. Dark grey bars and circles present mean insertion frequency and cell viability using crAAVS1. Light grey bars represent mean insertion frequency and cell viability using crIDTneg (IDT).

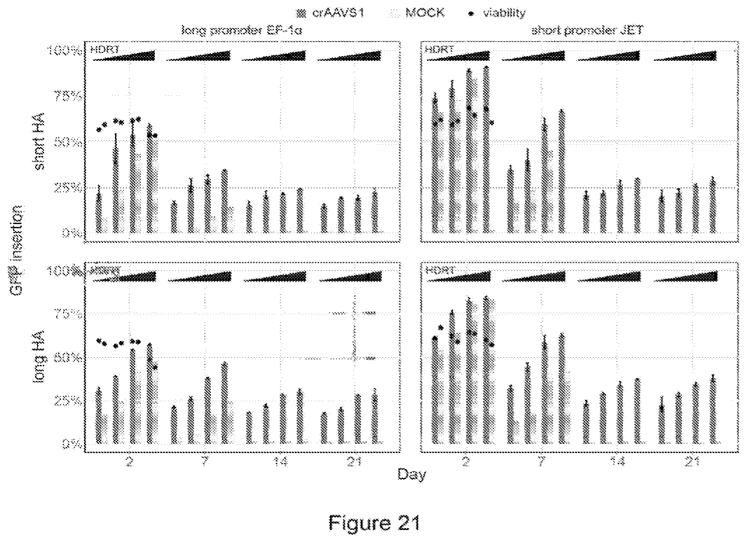

Subsequently, keeping the MAD7-RNP amounts constant, the effect of various homology arm lengths (100 vs 500 bp) and HDR template amounts (0.125 μg μL⁻¹, 0.25 μg μL⁻¹, 0.5 μg μL⁻¹, and 1 μg μL⁻¹) on the insertion efficiency was evaluated using JET and EF1α promoters. Up to 30% higher integration efficiency was observed with HDR templates flanked with HA of 500 compared to 100 base pairs. Moreover, the data showed improved insertion efficiencies with increasing amounts of HDR templates flanked with either 100 or 500 base pair HA but at the same time somewhat reduced cell viability (FIG. 21). Specifically, FIG. 21 shows GFP insertion efficiency at AAVS1 (n=3; Mean±SD) in T-cell leukemic cell line measured at days 2, 7, 14, and 21 post-transfection as a function of donor template amount. No transient GFP expression was observed at day 21 post-transfection. Cell viability (black circles) was measured at day 2 post-transfection. Top panels display GFP insertion efficiencies using donor template flanked with short homology arms (100 bp HA), and bottom panels donor template flanked with long homology arms (500 bp HA). Left panels display GFP insertion efficiencies using donor template containing EF-1α promoter (long, ˜2000 bp), and right panels donor template containing JET promoter (short, ˜1000 bp). Amount of donor template, represented by the gradient above the bars, increases from 0.125, 0.25, 0.5 to 1 μg μL⁻¹. Dark grey bars represent mean insertion frequency using crAAVS1. Light grey bars represent mean insertion frequency using crIDTneg (IDT).

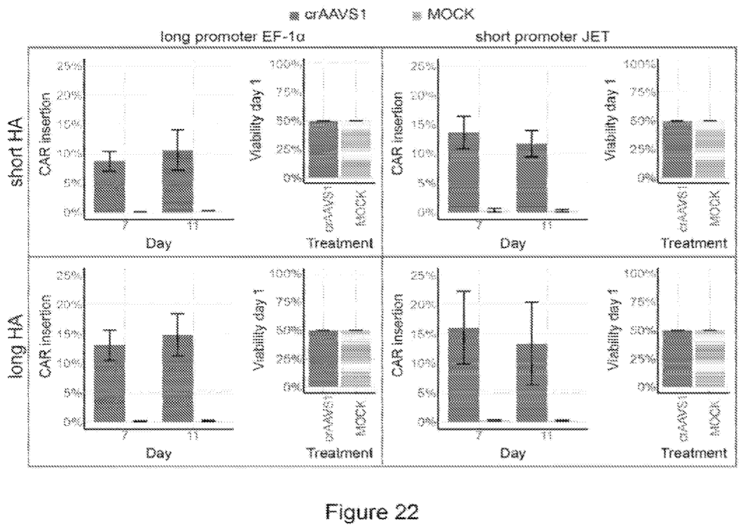

Next, using primary T-cells isolated from the human peripheral blood from three donors and a protocol selected from the experiments above, i.e., 150:100 pmol gNA:MAD7 RNP complex together with 1 μg μL⁻¹ HDR template, in combination with 100 μg μL⁻¹ poly-L-glutamic acid (PGA), integration efficiency of a clinically relevant CAR transgene containing JET or EF1α promoter flanked with HA of 100 or 500 base pairs and a bovine growth hormone derived polyadenylation sequence was analyzed. An anti-CD19 CAR with fully human variable regions (Hu19CAR), CD8α hinge and transmembrane domains, a CD28 costimulatory domain, and CD3ζ activation domain was used. Moderate insertion efficiency at AAVS1 but stable CAR expression of up to 14% and 16% was observed using HDR templates flanked with 100 and 500 base pair HA, respectively. The normalized cell viability measured 24 h post-transfection was in same cases relatively low, ranging from 22% with JET-500-CAR, 35% with JET-100-CAR, 43% with EF1a-100-CAR, to 55% with EF1a-500-CAR (FIG. 22). It is important to emphasize, that both CAR insertion efficiency and cell viability were higher in the treatment with PGA compared to the treatment without PGA (P≤0.05; data not shown). Specifically, FIG. 22 shows CAR insertion efficiency at AAVS1 (D=3; n=3; Mean±SD) in primary Pan T-cells measured at days 7 and 11 post-transfection. Cell viability was measured 24 hours post-transfection. Individual panels display CAR insertion efficiencies using donor template structure as described in FIG. 21. Amount of donor template, MAD7-RNP, and PGA was 1 μg μL⁻¹, 100:150 pmol MAD7:gNA, and 100 μg μL⁻¹, in that order. Nucleofection program P3-EH-115 for transfection of primary T-cells was used. D represents number of biological replicas, and n number of technical replicas per D. Dark grey bars represent mean insertion frequency using crAAVS1. Light grey bars represent mean insertion frequency using crIDTneg (IDT).

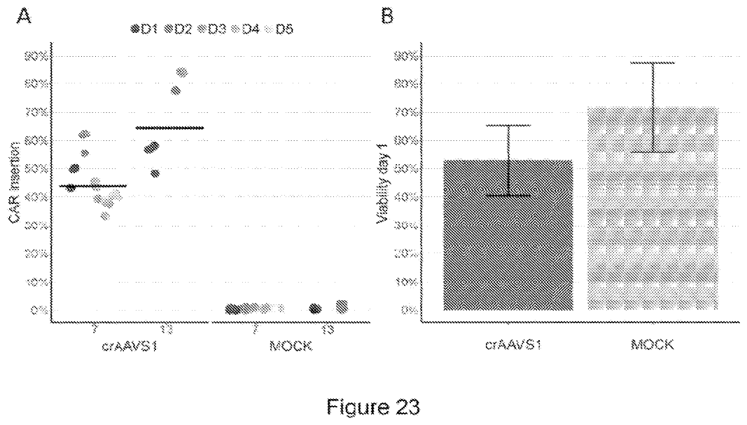

Multiple parameters were reevaluated to further optimize primary T-cell viability and CAR insertion efficiencies at AAVS1. Using Pan T-cells isolated from the blood from two donors, the effect of RNP amount with 100 μg μL⁻¹ PGA and EF1a-500-CAR template amount on CAR insertion efficiency and cell viability was tested (data not shown). Reducing the RNP amount to 75:50 pmol gNA:MAD7 RNP complex while increasing the donor template amount to 1.5 μg μL⁻¹ led to improved CAR insertion efficiencies without significantly affecting cell viability (P≥0.05; data not shown). In addition, using the abovementioned transfection conditions in combination with the cell recovery in a post-transfection cultivation medium pretreated with 2 μM M3814 resulted in nearly 5-times more efficient CAR insertion than other experiments (FIG. 23). The optimized CRISPR-MAD7 transfection protocol resulted in CAR insertion efficiency of up to 85% 13-days post-transfection (median 65%) together with the median normalized cell viability as high as 62% 24 hours post-transfection. Specifically, FIG. 23 shows CAR insertion efficiency at AAVS1 (D=5; n=3) in primary Pan T-cells measured at day 7 post-transfection, and re-measured in two biological replicas at day 13 post-transfection (D=2; n=3). Cell viability was measured 24 hours post-transfection (D=5; n=3; Mean±SD). Amount or concentration of donor template, MAD7-RNP, PGA, and M3814 was 1.5 μg μL⁻¹, 50:75 pmol MAD7:gNA, 100 μg μL⁻¹, and 2 μM, respectively. Nucleofection program P3-EH-115 for transfection of primary T-cells was used. D represents number of biological replicas, and n number of technical replicas per D. Dark grey bars represent mean insertion frequency using crAAVS1. Light grey bars represent mean insertion frequency using crIDTneg (IDT).

### IX. EQUIVALENTS

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The terms “a” and “an” and “the” and similar references in the context of describing the invention (especially in the context of the following claims) are to be construed to cover both the singular and the plural, unless otherwise indicated herein or clearly contradicted by context. For example, the term “a cell” includes a plurality of cells, including mixtures thereof. Where the plural form is used for compounds, salts, or the like, this is taken to mean also a single compound, salt, or the like.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1.-221. (canceled)

222. A composition comprising:
a. a nucleic acid-guided nuclease capable of binding to a compatible guide nucleic acid (gNA) comprising a spacer sequence complementary to a target nucleotide sequence within a target polynucleotide, for example a target polynucleotide of a genome of a human target cell and generating a strand break in one or both strands of the target polynucleotide; and
b. at least one additive that reduces non-homologous end joining (NHEJ)-based DNA repair and/or at least one additive that stabilizes the nucleic acid-guided nuclease system.

223. The composition of claim 222, further comprising a gNA, wherein the gNA is compatible with and capable of binding to and activating the nucleic acid-guided nuclease, wherein the gNA comprises:
a. a targeter nucleic acid comprising a targeter stem sequence and a spacer sequence, wherein the spacer sequence is complementary to a target nucleotide sequence within a target polynucleotide, for example a target polynucleotide of a genome of a human target cell; and
b. a modulator nucleic acid comprising a modulator stem sequence complementary to the targeter stem sequence, and, optionally, a 5′ sequence.

224. The composition of claim 222, wherein the additive that stabilizes the nuclease system comprises an anionic polymer.

225. The composition of claim 222, further comprising one or more human target cells.

226. The composition of claim 222, further comprising a donor template, wherein at least a portion of the donor template is capable of being inserted into the target polynucleotide at the site of cleavage.

227. The composition of claim 222, wherein the additive that reduces NHEJ comprises M3814.

228. The composition of claim 227, wherein the M3814 concentration is at least 0.1, 0.25, 0.5, 0.75, 1, 1.25, 1.5, 1.75, 2, 2.5, 3, or 4 and/or not more than 0.25, 0.5, 0.75, 1, 1.25, 1.5, 1.75, 2, 2.5, 3, 4, or 5 μM, for example 0.1-5 μM.

229. The composition of claim 222, wherein the nuclease comprises a Type V-A, V-B, V-C, V-D, or V-E nuclease, optionally wherein the nuclease comprises an amino acid sequence at least 80% identical to the amino acid sequence of MAD2 (SEQ ID NO: 38), MAD7 (SEQ ID NO: 37), ART2 (SEQ ID NO: 2), ART11 (SEQ ID NO: 11), or ART11* (SEQ ID NO: 36).

230. The composition of claim 223, wherein the targeter nucleic acid and the modulator nucleic acid are separate polynucleotides, capable of binding to and activating a nucleic acid-guided nuclease, that, in a naturally occurring system, is activated by a single crRNA in the absence of a tracrRNA.

231. The composition of claim 223, wherein the gNA comprises a spacer sequence comprising any one of SEQ ID NOs: 86-384.

232. The composition of claim 222, wherein:
the additive that stabilizes the nuclease system comprises an anionic polymer; or
the additive that stabilizes the nuclease system comprises 1,2,3-heptanetriol, 2-Amino-2-(hydroxymethyl)-1,3-propanediol (Tris), 3-(1-pyridino)-1-propane sulfonate (NDSB 201), 3-[(3-cholamidopropyl)dimethylammonio]-1-propanesulfonate (CHAPS), 6-aminocaproic acid, adenosine diphosphate (ADP), adenosine triphosphate (ATP), alpha-cyclodextrin, amidosulfobetaine-14 (ASB-14), ammonium acetate, ammonium nitrate, ammonium sulfate, arginine, arginine ethylester, barium chloride, barium iodide, benzamidine HCl, beta-cyclodextrin, beta-mercaptoethanol (BME), biotin, calcium chloride, cesium chloride, cesium sulfate, cetyltrimethylammonium bromide (CTAB), choline chloride, citric acid, cobalt chloride, copper (II) chloride, cyclohexanol, D-sorbitol, dimethylethylammoniumpropane sulfonate (NDSB 195), dithiothreitol (DTT), erythritol, ethanol, ethylene glycol, ethylene glycol-bis(βbeta-aminoethyl ether)-N,N,N′,N′-tetraacetic acid (EGTA), ethylenediaminetetraacetic acid (EDTA), formamide, gadolinium bromide, gamma butyrolactone, glucose, glutamic acid, glutamine, glycerol, glycine, glycine betaine, glycine-glycine-glycine, guanidine HCl, guanosine triphosphate (GTP), holmium chloride, imidazole, iron (III) chloride, Jeffamine M-600, lanthanum acetate, lauryl sulfobetaine, lauryldimethylamine N-oxide (LDAO), lithium sulfate, magnesium chloride, magnesium sulfate, manganese chloride, mannitol, N-(2-hydroxyethyl) piperazine-N′-(3-propanesulfonic acid) (EPPS), N-dodecyl beta-D-maltoside (DDM), N-ethylurea, n-hexanol, N-lauryl sarcoside, N-lauryl sarcosine, N-methylformamide, N-methylurea, n-octyl-b-D-glucoside (OG: Octyl glucoside), n-penthanol, nickel chloride, non-detergent sulfo betaine (NDSB), Nonidet P40 (NP40), octyl beta-D-glucopyranoside, poly-L-glutamic acid, polyethylene glycol (for example, PEG 300, PEG 3350, PEG 4000), polyethyleneglycol lauryl ether (Brij 35), polyoxyethylene (2) oleyl ether (Brij 93), polyoxyethylene cetyl ether (Brij 56), polyvinylpyrrolidone 40 (PVP40), potassium chloride, potassium citrate, potassium nitrate, proline, putrescine, spermidine, spermine, riboflavin, samarium bromide, sarcosine, sodium acetate, sodium chloride, sodium dodecyl sulfate (SDS), sodium fluoride, sodium iodide, sodium lauroyl sarcosinate (Sarkosyl), sodium malonate, sodium molybdate, sodium selenite, sodium sulfate, sodium thiocyanate, sucrose, taurine, trehalose, tricine, triethylamine, trimethylamine N-oxide (TMAO), tris(2-carboxyethyl)phosphine (TCEP), Triton X-100, Tween 20, Tween 60, Tween 80, urea, vitamin B12, xylitol, yttrium chloride, yttrium nitrate, zinc chloride, Zwittergent 3-08, Zwittergent 3-14, or a combination thereof.

232. The composition of claim 231, wherein the additive that stabilizes the nuclease system comprises poly-L-glutamic acid (PGA), optionally wherein the PGA is present at a concentration of at least 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, or 4.5 and/or not more than 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, or 5 μg μL⁻¹ per pmol RNP complex, for example 0.01-5 μg μL⁻¹ per pmol RNP complex.

233. The composition of claim 226, wherein the donor template comprises a transgene, optionally wherein the transgene comprises a fluorescent protein, a bioluminescent protein, an apoptotic switch, a cytokine, an interleukin, a gene circuit, a fusion protein, a CAAR, or a CAR component.

234. The composition of claim 225, wherein the human target cells comprise an immune cell or a stem cell, optionally wherein:
the immune cell comprises a neutrophil, eosinophil, basophil, mast cell, monocyte, macrophage, dendritic cell, natural killer cell, a lymphocyte, a T cell, or a CAR-T cell; and/or
the stem cell comprises a human pluripotent stem cell, multipotent stem cell, embryonic stem cell, induced pluripotent stem cell, or hematopoietic stem cell.

235. A method for editing a target polynucleotide in the genome of a human target cell comprising:
a. contacting the target polynucleotide with a nucleic acid-guided nuclease system comprising:
i. a nucleic acid-guided nuclease; and
ii. a guide nucleic acid (gNA) compatible with and capable of binding to and activating the nucleic acid-guided nuclease, wherein the gNA comprises:
1. a targeter nucleic acid comprising a targeter stem sequence and a spacer sequence, wherein the spacer sequence is complementary to a target nucleotide sequence within a target polynucleotide, for example a target polynucleotide of a genome of a human target cell; and
2. a modulator nucleic acid comprising a modulator stem sequence complementary to the targeter stem sequence, and, optionally, a 5′ sequence; and
b. contacting the cell with at least one additive that reduces non-homologous end joining (NHEJ)-based DNA repair and/or combining the nucleic acid-guided nuclease system, before delivering, with at least one additive that stabilizes the nucleic acid-guided nuclease system.

236. The method of claim 235, wherein the additive that stabilizes the nucleic acid-guided nuclease system is combined with the gNA prior to introduction of the nuclease.

237. The method of claim 235, wherein the nuclease system further comprises a donor template, wherein at least a portion of the donor template is capable of being inserted into the target polynucleotide at the site of cleavage.

238. The method of claim 235, wherein the additive that reduces NHEJ is present in the recovery medium to which cells are added after delivery of the nuclease system and/or donor template.

239. The method of claim 235, wherein the additive that reduces NHEJ comprises M3814.

240. The method of claim 239, wherein the M3814 concentration is at least 0.1, 0.25, 0.5, 0.75, 1, 1.25, 1.5, 1.75, 2, 2.5, 3, or 4 and/or not more than 0.25, 0.5, 0.75, 1, 1.25, 1.5, 1.75, 2, 2.5, 3, 4, or 5 μM, for example 0.1-5 μM.

241. The method of claim 235, wherein the nuclease comprises a Type V-A, V-B, V-C, V-D, or V-E nuclease, optionally wherein the nuclease comprises an amino acid sequence at least 80% identical to the amino acid sequence of MAD2 (SEQ ID NO: 38), MAD7 (SEQ ID NO: 37), ART2 (SEQ ID NO: 2), ART11 (SEQ ID NO: 11), or ART11* (SEQ ID NO: 36).

242. The method of claim 235, wherein the targeter nucleic acid and the modulator nucleic acid are separate polynucleotides, capable of binding to and activating a nucleic acid-guided nuclease, that, in a naturally occurring system, is activated by a single crRNA in the absence of a tracrRNA.

243. The method of claim 235, wherein the gNA comprises a spacer sequence comprising any one of SEQ ID NOs: 86-384.

244. The method of claim 236, wherein:
the additive that stabilizes the nuclease system comprises an anionic polymer; or
the additive that stabilizes the nuclease system comprises 1,2,3-heptanetriol, 2-Amino-2-(hydroxymethyl)-1,3-propanediol (Tris), 3-(1-pyridino)-1-propane sulfonate (NDSB 201), 3-[(3-cholamidopropyl)dimethylammonio]-1-propanesulfonate (CHAPS), 6-aminocaproic acid, adenosine diphosphate (ADP), adenosine triphosphate (ATP), alpha-cyclodextrin, amidosulfobetaine-14 (ASB-14), ammonium acetate, ammonium nitrate, ammonium sulfate, arginine, arginine ethylester, barium chloride, barium iodide, benzamidine HCl, beta-cyclodextrin, beta-mercaptoethanol (BME), biotin, calcium chloride, cesium chloride, cesium sulfate, cetyltrimethylammonium bromide (CTAB), choline chloride, citric acid, cobalt chloride, copper (II) chloride, cyclohexanol, D-sorbitol, dimethylethylammoniumpropane sulfonate (NDSB 195), dithiothreitol (DTT), erythritol, ethanol, ethylene glycol, ethylene glycol-bis(βbeta-aminoethyl ether)-N,N,N′,N′-tetraacetic acid (EGTA), ethylenediaminetetraacetic acid (EDTA), formamide, gadolinium bromide, gamma butyrolactone, glucose, glutamic acid, glutamine, glycerol, glycine, glycine betaine, glycine-glycine-glycine, guanidine HCl, guanosine triphosphate (GTP), holmium chloride, imidazole, iron (III) chloride, Jeffamine M-600, lanthanum acetate, lauryl sulfobetaine, lauryldimethylamine N-oxide (LDAO), lithium sulfate, magnesium chloride, magnesium sulfate, manganese chloride, mannitol, N-(2-hydroxyethyl) piperazine-N′-(3-propanesulfonic acid) (EPPS), N-dodecyl beta-D-maltoside (DDM), N-ethylurea, n-hexanol, N-lauryl sarcoside, N-lauryl sarcosine, N-methylformamide, N-methylurea, n-octyl-b-D-glucoside (OG: Octyl glucoside), n-penthanol, nickel chloride, non-detergent sulfo betaine (NDSB), Nonidet P40 (NP40), octyl beta-D-glucopyranoside, poly-L-glutamic acid, polyethylene glycol (for example, PEG 300, PEG 3350, PEG 4000), polyethyleneglycol lauryl ether (Brij 35), polyoxyethylene (2) oleyl ether (Brij 93), polyoxyethylene cetyl ether (Brij 56), polyvinylpyrrolidone 40 (PVP40), potassium chloride, potassium citrate, potassium nitrate, proline, putrescine, spermidine, spermine, riboflavin, samarium bromide, sarcosine, sodium acetate, sodium chloride, sodium dodecyl sulfate (SDS), sodium fluoride, sodium iodide, sodium lauroyl sarcosinate (Sarkosyl), sodium malonate, sodium molybdate, sodium selenite, sodium sulfate, sodium thiocyanate, sucrose, taurine, trehalose, tricine, triethylamine, trimethylamine N-oxide (TMAO), tris(2-carboxyethyl)phosphine (TCEP), Triton X-100, Tween 20, Tween 60, Tween 80, urea, vitamin B12, xylitol, yttrium chloride, yttrium nitrate, zinc chloride, Zwittergent 3-08, Zwittergent 3-14, or a combination thereof.

245. The method of claim 244, wherein the additive that stabilizes the nuclease system comprises poly-L-glutamic acid (PGA), optionally wherein the PGA is present at a concentration of at least 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, or 4.5 and/or not more than 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, or 5 μg μL⁻¹ per pmol RNP complex, for example 0.01-5 μg μL⁻¹ per pmol RNP complex.
